(19) United States
(12) Patent Application Publication
(10) Pub. No.: US 2024/0226264 A1
(43) Pub. Date: Jul. 11, 2024

(54) Porcine Circovirus Type 3 (PCV3) Vaccines, and Production and Uses Thereof

(71) Applicants: Boehringer Ingelheim Vetmedica GmbH, Ingelheim am Rhein (DE); lowa State University Research Foundation, Inc., Ames, IA (US)

(72) Inventors: Arun IYER, Ames, IA (US); Luis Alejandro Hernandez, Ames, IA (US); Abby Patterson, Story City, IA (US); Bailey Arruda, Ames, IA (US); Luis Gabriel Gimenez-Lirola, Ames, IA (US); Dave Michael Anstrom, Ames, IA (US); Eric M. Vaughn, Ames, IA (US); Pablo E Pineyro Pineiro, Ames, IA (US); Troy James Kaiser, Dearborn, MO (US); Joseph Ralph Hermann, Waukee, IA (US)

(21) Appl. No.: 18/396,787

(22) Filed: Dec. 27, 2023

Related U.S. Application Data
(63) Continuation of application No. 18/058,657, filed on Nov. 23, 2022, now Pat. No. 11,896,659.
Application No. 18/058,657 is a continuation of application No. 16/841,485, filed on Apr. 6, 2020, now Pat. No. 11,701,419.
(60) Provisional application No. 62/829,400, filed on Apr. 4, 2019.

(51) Int. Cl. A61K 39/12; A61K 39/00; A61K 39/23; A61P 31/20; A61P 37/04

(52) U.S. Cl. CPC A61K 39/12; A61K 39/23; A61P 31/20; A61P 37/04; A61K 2039/545; A61K 2039/552; A61K 2039/70; C12N 2750/10034; C12N 2750/10071; C12N 2750/14034

(57) ABSTRACT

The present invention relates to the use of an immunogenic composition that comprises a porcine circovirus type 3 (PCV3) antigen for treatment of several clinical manifestations (diseases). Preferably, the clinical manifestations are associated with a PCV3 infection.

### RELATED APPLICATIONS AND INCORPORATION BY REFERENCE

This application claims priority to U.S. patent application Ser. No. 18/058,657 filed on Nov. 23, 2022, which claims priority to U.S. patent application Ser. No. 16/841,485 filed on Apr. 6, 2020, which claims priority to U.S. provisional application 62/829,400 filed on Apr. 4, 2019, the entire contents of each application are hereby incorporated by reference herein. Reference is also made to WO 2006/072065 and U.S. Pat. Nos. 6,103,526; 9,610,345; 9,669,087 and 10,450,351; the disclosures of which are hereby incorporated by reference in their entireties.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### STATEMENT REGARDING SEQUENCE LISTING

The Sequence Listing associated with this application is provided in XML format in lieu of a paper copy, and is hereby incorporated by reference into the specification. The name of the XML file containing the Sequence Listing is BI19-AH009-US-3_SL.xml. The XML file is 191 Kbytes; it was created on 3 Nov. 2023; and it is being submitted electronically via EFS-Web, concurrent with the filing of the specification.

### FIELD OF THE INVENTION

Disclosed herein is a recombinant baculovirus vector containing a polynucleotide encoding Porcine Circovirus Type 3 (PCV3) ORF2. Also disclosed herein are compositions and vaccines produced from the baculovirus derived PCV3 ORF2 and BaculoG/PCV3 ORF2. Also disclosed is a recombinant baculovirus vector containing a mutated polynucleotide encoding Porcine Circovirus Type 3 (PCV3) ORF2. Also disclosed are compositions and vaccines produced from the baculovirus derived mutated PCV3 ORF2 and BaculoG/PCV3 ORF2.

### BACKGROUND OF THE INVENTION

Porcine circovirus type 3 (PCV3) is a non-enveloped, icosahedral single-stranded DNA (ssDNA) virus belonging to the genus Circovirus in the family Circoviridae. The genome encodes for two major open reading frames (ORFs) where ORF1 encodes a replication-associated protein (rep) and ORF2 encodes the viral capsid (cap) protein, which determines the antigenic characteristics of the virus. PCV3 is genetically distinct from porcine circovirus type 2 (PCV2); specifically, there is only 48% amino acid identity in the rep gene and 26% amino acid identity in the cap gene between the two viruses.

PCV3 was originally reported in 2016 in the U.S., Palinski, Rachel, et al. “A Novel Porcine Circovirus Distantly Related to Known Circoviruses Is Associated with Porcine Dermatitis and Nephropathy Syndrome and Reproductive Failure.” Journal of Virology, vol. 91, no. 1, 26 Oct. 2016. The virus has since been identified worldwide including Germany, Japan, Korea, Russia, China, Thailand, Italy, Spain, Denmark, South Korea, Poland, Brazil, Columbia, India, Serbia and Sweden. While testing is limited to date, the finding of PCV3 in retrospective samples indicates that the virus was likely circulating in swine populations worldwide decades prior to the initial 2017 reports. It is hypothesized that as testing increases, PCV3 will be identified in more countries and in older samples.

Additionally, Chinese patent application CN109207441A entitled, “3 type Cap protein of recombinant baculovirus expression pig circular ring virus and its construction method and primer,” claims priority to CN201810912587.1A, filed Aug. 12, 2018. It describes the construction of Baculovirus expression of PCV3 ORF2 for the manufacturing of 3 type Cap proteins of pig circular ring virus.

CN109207441A entitled, “3 type Cap protein of recombinant baculovirus expression pig circular ring virus and its construction method and primer,” claims priority to CN201810912587.1A, filed Aug. 12, 2018. It describes the administration of the Baculovirus expressed PCV3 ORF2 in mice and provides ELISA scroconversion data.

CN109207522A entitled, “It expresses 3 type of pig circular ring virus and truncates Cap protein of recombinant baculovirus and its construction method and primer,” claims priority to CN201810912585.2A, filed Aug. 12, 2018. It describes Baculovirus-truncated CAP/ORF2, administration in mice, and provides ELISA seroconversion data.

Additionally, U.S. Pat. No. 10,450,351 (i.e., application Ser. No. 15/768,356) entitled, “Porcine Circovirus Type 3 Immunogenic Compositions and Methods of Making and Using the Same,” was first published as US 2018/0305410 A1 on Oct. 25, 2018. It claims priority to provisional patent application 62/242,866, filed Oct. 16, 2015. (Inventor Ben Hause, assigned to Kansas State University Research Foundation. See also Palinski, Rachel, et al. Journal of Virology, vol. 91, no. 1, 26 Oct. 2016, doi: 10.1128/jvi.01879-16. Published online Oct. 26, 2016. It relates to PCV3 from tissues “collected from four sows from a farm with chronic poor reproductive performance which died acutely with clinical symptoms consistent with PDNS.” While the patent application does not say where the farm was located, it does describe that immunohistochemistry (IHC) and quantitative PCR (qPCR) were negative for PCV2, porcine reproductive and respiratory syndrome virus (PRRSV), and influenza A virus (IAV) on sows and mummified, stillborn and/or weak fetuses. This patent application describes isolation of the virus, but not of a propagating cell culture.

Examples in the '351 patent describe the qPCR detection of the PCV3 capsid gene, isolating the virus, cloning the PCV3 capsid protein, developing an anti-PCV3 capsid monoclonal antibody, PCV3 detection, and development of a recombinant PCV3 capsid ELISA. However, no vaccine studies or data are described.

Recently, an article was published describing intranasally inoculating 4- and 8-week-old specific-pathogen-free piglets with an infectious PCV3 DNA clone to evaluate PCV3 pathogenesis. However, no discussion of vaccines to prevent PCV3 infection was made. Jiang, Haijun, et al. “Induction of Porcine Dermatitis and Nephropathy Syndrome in Piglets by Infection with Porcine Circovirus Type 3.” Journal of Virology, vol. 93, no. 4, 28 Nov. 2018, doi: 10.1128/jvi.02045-18.

Citation or identification of any document in this application is not an admission that such document is available as prior art to the present invention.

### SUMMARY OF THE INVENTION

Disclosed are PCV3 ORF2 antigenic proteins and variants thereof that are useful in the vaccination of or treatment of animals, in particular swine.

Typically, the swine is a pig.

In some aspects of the present invention, the animal is a piglet. Typically, the piglet is not older than 15 weeks of age, or not older than 6 weeks of age, or not older than 3 weeks of age, or not older than 2 weeks of age, or not older than 1 week of age sow.

In some aspects of the present invention, swine is a sow or a gilt.

In some aspects of the present invention the swine is a sow or gilt (i.e. a sow that has not farrowed) that is less than 1 year in age, typically more than 4 months and less than 1 year in age, typically more than 5 months and less than 1 year in age, typically more than 6 months and less than 1 year in age, typically between 4 to 8 months in age, typically between 5 to 8 months in age, typically between 5 to 7 months in age, typically between 5 to 6 months in age.

In some aspects of the present invention the swine is a pregnant sow that is less than 1 year in age, typically more than 4 months and less than 1 year in age, typically more than 5 months and less than 1 year in age, typically more than 6 months and less than 1 year in age.

In some aspects of the present invention the swine is a pre-breeding gilt that is less than 1 year in age, typically more than 4 months and less than 1 year in age, typically more than 5 months and less than 1 year in age, typically more than 6 months and less than 1 year in age, typically between 4 to 8 months in age, typically between 5 to 8 months in age, typically between 5 to 7 months in age, typically between 5 to 6 months in age.

Disclosed is the development of baculovirus derived PCV3 ORF2, expressed from “BaculoG/PCV3 ORF2”, compositions, and three vaccines: BaculoG/PCV3 ORF2, P9; live, adjuvanted with 50% ISA 207VG vaccine; BaculoG/PCV3 ORF2, P9; live, adjuvanted with 20% carbopol vaccine, and control BaculoG/no insert, P4; live, adjuvanted with 20% carbopol vaccine. Data showing efficacy of the vaccines to prevent PCV3 disease was provided.

Also disclosed is the development of baculovirus derived PCV3 ORF2 derived from killed virus.

Also disclosed is the development of baculovirus derived PCV3 ORF2 derived from mutated killed virus.

In a first aspect, the present invention thus relates to a composition comprising a PCV3 ORF2 protein, preferably an antigenic PCV3 ORF2 protein (a PCV3 ORF2 antigen). Said composition is also termed “the composition of the present invention” hereinafter. It also understood that the term “composition of the present invention”, as described herein, is equivalent to “composition of the disclosure”.

Preferably, the composition of the present invention further comprises a veterinary acceptable carrier selected from the group consisting of: a solvent, a dispersion media, a coating, a stabilizing agent, a diluent, a preservative, an anti-microbial agent, an antifungal agent, an isotonic agent, an adsorption delaying agent, an adjuvant, cell culture supernatant, a stabilizing agent, a viral vector, an expression vector, an immunomodulatory agent, and/or any combination thereof.

The present disclosure further relates to a porcine circovirus type 3 (PCV3) ORF2 protein; and a veterinary-acceptable carrier comprising a solvent, a dispersion media, a coating, a stabilizing agent, a diluent, a preservative, an anti-microbial agent, an antifungal agent, an isotonic agent, an adsorption delaying agent, an adjuvant, cell culture supernatant, a stabilizing agent, a viral or expression vector, an immunomodulatory agent and/or any combination thereof.

In one embodiment, the veterinary-acceptable carrier comprises an adjuvant, immunomodulatory agent, cell culture supernatant, viral or expression vector or any combination thereof. In another embodiment, the veterinary-acceptable carrier comprises an adjuvant.

The PCV3 ORF2 can be from group a1, b1 or b2 (using the subtyping designation of Fux et al., “Full genome characterization of porcine circovirus type 3 isolates reveals the existence of two distinct groups of virus strains,” Virology Journal (2018) 15:25, DOI 10.1186/s12985-018-0929-3 (incorporated herein by reference); see, e.g., Table 4). Thus, the PCV3 as mentioned herein is any phylogenetic clade of PCV3 or combination of clades or preferably selected from the group consisting of PCV3a and PCV3b, and most preferably selected from the group consisting of PCV3a1, PCV3b1, PCV3b2 and PCV3c. The composition of the present invention thus preferably comprises a PCV3 ORF2 protein selected from the group consisting of PCV3a ORF2 protein and PCV3b ORF2 protein, or most preferably comprises a PCV3 ORF2 protein is any phylogenetic clade of PCV3 or combination of clades or selected from the group consisting of PCV3a1 ORF2 protein, PCV3b1 ORF2 protein and PCV3b2 ORF2 protein. In another embodiment, the PCV3 ORF2 protein comprises or consists of an amino acid sequence encoded by a polynucleotide sequence having at least 90%, or at least 91%, or at least 92%, or at least 93%, or at least 94%, or at least 95%, or at least 96%, or at least 97%, or at least 98%, or at least 99%, or 100% sequence identity or sequence homology with SEQ ID NO: 1. Preferably the PCV3 ORF2 protein comprises or consists of an amino acid sequence having at least 90%, or at least 91%, or at least 92%, or at least 93%, or at least 94%, or at least 95%, or at least 96%, or at least 97%, or at least 98%, or at least 99%, or 100% sequence identity with the sequence of SEQ ID NO: 4. According to a particular preferred aspect, the PCV3 ORF2 protein is a recombinant protein, or most preferably a recombinant baculovirus expressed protein. Thus, the composition preferably comprises recombinant PCV3 ORF2 protein, or most preferably comprises baculovirus expressed PCV3 ORF2 protein.

In another embodiment, the PCV3 ORF2 protein is a recombinant PCV3 ORF2 protein from expression thereof by an expression vector, comprising a polynucleotide sequence that encodes the PCV3 ORF2 protein. Advantageously, the expression vector is a baculovirus.

In yet another embodiment, the composition further comprises a PCV2 ORF protein, which may be from expression by an expression vector, comprising a polynucleotide sequence that encodes the PCV2 ORF2 protein. Advantageously, the expression vector is a baculovirus.

Furthermore, the composition may further comprise at least one additional antigen of an additional porcine pathogen. The additional antigen or antigens of porcine pathogens comprises a PRRSV antigen, a Mycoplasma hyopneumoniae bacterin antigen, a Mycoplasma hyopneumoniae supernatant antigen, an Aujeszky's disease or pseudorabies virus antigen, a IAV antigen, a swine fever antigen (classical or African or combination thereof), an Actinobacillus pleuropneumoniae antigen, an Escherichia coli antigen, a porcine parvovirus (PPV) antigen, a Pasteurella multocida antigen, a Erysipelothrix rhusiopathiae antigen or a Mycoplasma hyorhinis antigen.

In another embodiment, PCV3 ORF2 protein is present in an amount of 0.2 to about 400 μg/ml, or 2 to about 400 μg/ml, or 4 to about 400 μg/ml, or 8 to about 400 μg/ml, or about 0.3 to about 200 μg/ml, or 2 to about 200 μg/ml, or 4 to about 200 μg/ml, or 8 to about 200 μg/ml, or about 0.35 to about 100 μg/ml, or 2 to about 100 μg/ml, or 4 to about 100 μg/ml, or 8 to about 100 μg/ml, or about 0.4 to about 50 μg/ml, or 2 to about 50 μg/ml, or 4 to about 50 μg/ml, or 8 to about 50 μg/ml, or about 0.45 to about 30 μg/ml, or about 0.6 to about 15 μg/ml, or about 0.75 to about 8 μg/ml, or about 1.0 to about 6 μg/ml, or about 1.3 to about 3.0 μg/ml, or about 1.4 to about 2.5 μg/ml, or about 1.5 to about 2.0 μg/ml, or about 1.6 μg/ml. In a particular embodiment, the composition may have PCV3 ORF2 protein in an amount in a range from about 1.5 to about 2.0 μg/ml of the composition. For example, in an embodiment a 1 ml dose of the composition may include about 1.6 μg of PCV3 ORF2 protein.

In another embodiment, PCV3 ORF2 protein or total PCV2 and PCV3 ORF2 proteins are present in an amount of about 0.2 to about 400 μg/dose, or 2 to about 400 μg/dose, or 4 to about 400 μg/dose, or 8 to about 400 μg/dose, or about 0.3 to about 200 μg/dose, or 2 to about 200 μg/dose, or 4 to about 200 μg/dose, or 8 to about 200 μg/dose, or about 0.35 to about 100 μg/dose, or 2 to about 100 μg/dose, or 4 to about 100 μg/dose, or 8 to about 100 μg/dose, or about 0.4 to about 50 μg/dose, or 2 to about 50 μg/dose, or 4 to about 50 μg/dose, or 8 to about 50 μg/dose, or about 0.45 to about 30 μg/dose, or about 0.6 to about 15 μg/dose, or about 0.75 to about 8 μg/dose, or about 1.0 to about 6 μg/dose, or about 1.3 to about 3.0 μg/dose, or about 1.4 to about 2.5 μg/dose, or about 1.5 to about 2.0 μg/dose, or about 1.6 μg/dose. In a particular embodiment, the composition may have a total PCV3 and PCV2 ORF2 protein in an amount in a range from about 1.5 to about 2.0 μg/ml of the composition. For example, in an embodiment a 1 ml dose of the composition may include about 1.6 μg of combined PCV3 and PCV2 ORF2 protein.

In another embodiment, the adjuvant comprises aluminum hydroxide; aluminum phosphate; a saponin; Quil AR; QS-21®; GPI-0100; a water-in-oil emulsion; an oil-in-water emulsion; a water-in-oil-in-water emulsion; an emulsion based on light liquid paraffin oil or European Pharmacopca type adjuvant; an isoprenoid oil; squalane; squalene oil resulting from oligomerization of alkenes or isobutene or decene; (an) ester(s) of acid(s) or of alcohol(s) containing a linear alkyl group; plant oil(s); ethyl oleate; propylene glycol di-(caprylate/caprate); glyceryl tri-(caprylate/caprate); propylene glycol dioleate; (an) ester(s) of branched fatty acid(s) or alcohol(s); isostearic acid ester(s); nonionic surfactant(s); (an) ester(s) of sorbitan or of mannide or of glycol or of polyglycerol or of propylene glycol or of oleic, or isostearic acid or of ricinoleic acid or of hydroxystearic acid, optionally ethoxylated, anhydromannitol oleate; polyoxypropylene-polyoxyethylene copolymer blocks, a Pluronic® product, a carbopol; Carbopol® 974P; Carbopol® 934P; Carbopol® 971P; a polymer of acrylic or methacrylic acid; copolymer of maleic anhydride and alkenyl derivative; a polymer of acrylic or methacrylic acid which is cross-linked; a polymer of acrylic or methacrylic acid which is cross-linked with a polyalkenyl ether of sugar or polyalcohol; a carbomer; an acrylic polymer cross-linked with a polyhydroxylated compound having at least 3 and not more than 8 hydroxyl groups with hydrogen atoms of at least three hydroxyls optionally or being replaced by unsaturated aliphatic radicals having at least 2 carbon atoms with said radicals containing from 2 to 4 carbon atoms such as vinyls, allyls and other ethylenically unsaturated groups and the unsaturated radicals may themselves contain other substituents, such as methyl; RIBI® adjuvant system; Block co-polymer; SAF-M; monophosphoryl lipid A; Avridine lipid-amine adjuvant; heat-labile enterotoxin from E. coli (recombinant or otherwise); cholera toxin; MONTANIDE™ IMS 1314, or muramyl dipeptide.

In yet another embodiment, there may be about 50 μg to about 2000 μg of adjuvant; or wherein adjuvant present in an amount about 250 μg/ml dose of the composition, or wherein the adjuvant is present in an amount of about 100 μg to about 10 mg per dose; or wherein the adjuvant is present in an amount of about 500 μg to about 5 mg per dose; the adjuvant is present in an amount of about 750 μg to about 2.5 mg per dose; or the adjuvant is present in an amount of about 1 mg per dose. In a particular embodiment, the composition may include adjuvant in a range from about 750 μg to about 2.5 mg per dose of the composition. For example, in an embodiment a dose of the composition may include about 1 mg of adjuvant.

In one embodiment, the immunomodulatory agent comprises interleukin(s), interferon(s), or other cytokine(s).

The dosage of the antibiotic(s) may be from about 1 ug/ml to about 60 μg/ml of antibiotic(s), or less than about 30 μg/ml of antibiotic(s). For example, an embodiment of the composition may include less than about 30 μg/ml of antibiotic(s).

In one embodiment, the antibiotic(s) comprise Gentamicin.

A composition of the disclosure may comprise (i) PCV3 ORF2 protein, (ii) at least a portion of baculovirus that expressed said PCV3 ORF2 protein, (iii) a portion of cell culture of cells that were infected or transfected with recombinant baculovirus that expressed said PCV3 ORF2 protein, (iv) inactivating agent or inactivating agent comprising binary ethyleneimine (BEI), (v) sodium thiosulfate or sodium thiosulfate in equivalent amounts to inactivating agent or BEI; (vi) adjuvant or adjuvant comprising Carbopol® or Carbopol® 971, and (vii) phosphate salt in a physiologically acceptable concentration. In one embodiment, about 90% of the components (i) to (iii) may have a size smaller than 1 μm and the pH of said composition is adjusted to about 6.5 to 7.5. In another embodiment, the BEI is from the cell culture having been treated with about 2 to 8 or about 5 mM BEI to inactivate the baculovirus. In another embodiment, the composition contains about 2 to 8 or about 5 mM BEI The composition may contain about 1 mg of the Carbopol® or Carbopol® 971. For example, an embodiment of the composition may include a cell culture that has been treated with BEI at a concentration of about 5 mM to inactivate the baculovirus. In some embodiments, a dose of the composition may include residual BEI and/or about 1 mg of Carbopol®, Carbopol 971®, or a combination thereof.

Any composition of the disclosure may be formulated and/or packaged for a single dose or one shot administration, as well as a multi-dose regimen. It is presumed that a single administration can overcome the presence of maternally derived antibodies.

In one embodiment, the composition may be a PCV3 and PPV (advantageously packaged in a VLP) and/or PRRSV advantageously for use in breeding age sows/gilts. In such an embodiment, one or more doses for administration is contemplated.

According to another aspect the composition of the present invention is an immunogenic composition.

The invention further provides the composition of the present invention for use as a medicament.

Further, the composition of the present invention is provided for use as a vaccine.

According to a particular preferred aspect, the composition of the present invention is for use in method for eliciting an immune response or an immunologic response or a protective immune or immunological response against (i) PCV3 and/or (ii) PCV2 and PCV3 and/or (iii) PCV3 and other porcine pathogens and/or (iv) PCV3, PCV2 and other porcine pathogens.

According to another preferred aspect, the composition of the present invention is for use in a method of reducing or preventing the clinical signs or disease caused by an infection with PCV3 in an animal or for use in a method of treating or preventing an infection with PCV3 in an animal, and wherein said animal is preferably a pig.

Further, the composition of the present invention is provided for use in a method for inducing an immune response against PCV3 in a pig, in particular in a preferably pregnant sow.

According to still another aspect, the composition of the present invention is provided for use in a method of reducing or preventing the clinical signs or disease caused by an infection with a PCV3 in a piglet, wherein the piglet is to be suckled by a sow to which the composition has been administered.

Thus, the present invention further provides the composition of the present invention for use in a method of reducing or preventing the clinical signs or disease caused by an infection with a PCV3 in a piglet, wherein the piglet is to be suckled by a sow to which the composition of the present invention has been administered, and wherein preferably said sow to which the composition has been administered is a sow to which the immunogenic composition has been administered while said sow has been pregnant, in particular with said piglet, or a pre-breeding gilt.

Preferably, the composition of the present invention for use in any one of the aforementioned methods is administered intramuscularly or intradermally, in particular to said sow.

The present disclosure also encompasses a method for eliciting an immune response or an immunological response or a protective immune or immunological response against (i) PCV3 and/or (ii) PCV2 and PCV3 and/or (iii) PCV3 and another porcine pathogen and/or (iv) PCV3, PCV2 and another porcine pathogen, comprising administering to an animal any of the herein disclosed compositions. The animal may be a porcine. Advantageously, the porcine may be a pig or a piglet or a sow. The pig or piglet may be not older than 15 weeks of age, or not older than 6 weeks of age, or not older than 3 weeks of age, or not older than 2 weeks of age, or not older than 1 week of age. The administration may occur within at least 1 or 2 or 3 weeks of exposure to virulent Porcine Circovirus. The administration may occur within at least 1 or 2 or 3 weeks of exposure to virulent Porcine Circovirus. For some aspects, the administration may comprise a single, one shot administration; or a single, one dose administration of the protein of the present invention or the composition of the present invention; and not a multi-shot or multi-dose regimen. For some aspects, the administration may comprise a multi-shot or multi-dose regimen of the protein of the present invention or the composition of the present invention.

Further, the present invention provides a method of immunizing a subject comprising administering to the subject the composition of the present invention.

Further, the present invention provides a method of immunizing swine against a clinical disease caused by at least one pathogen in said animal, said method comprising the step of administering to the animal the composition of the present invention, wherein said immunogenic composition fails to cause clinical signs of infection but is capable of inducing an immune response that immunizes the animal against pathogenic forms of said at least one pathogen, and wherein said at least one pathogen is preferably PCV3.

Further, the present invention provides a method for inducing the production of antibodies specific for PCV3 in a sow, wherein said method comprises administering the composition of the present invention. The sow can be a pregnant sow. Alternatively, the sow can be a gilt (i.e. a sow that has not farrowed)—preferably a pre-breeding gilt.

Further, the present invention provides a method of reducing or preventing the clinical signs or clinical symptoms caused by an infection with a PCV3 in a piglet, wherein said method comprises
administering the composition of the present invention to a sow, and
allowing said piglet to be suckled by said sow,
and wherein said sow is preferably a sow being pregnant, in particular with said piglet.

Preferably, the latter above-mentioned methods comprise the steps of
administering the composition of the present invention to a sow being pregnant with said piglet,
allowing said sow to give birth to said piglet, and
allowing said piglet to be suckled by said sow.

Further, the present invention provides a method of reducing the clinical signs and/or clinical symptoms caused by an infection with a porcine epidemic diarrhea virus (PEDV) in a piglet, wherein the piglet is to be suckled by a sow to which the composition of the present invention has been administered.

Preferably, in any one of the aforementioned methods, where applicable, the composition of the present is administered intramuscularly or intradermally, in particular to said sow.

According to another preferred aspect, the immunogenic composition of the present invention is administered twice, in particular intramuscularly or intradermally, to said sow.

In another preferred aspect, the clinical signs, as mentioned herein, are selected from the group consisting of reduction of average daily weight gain and mortality.

In a further preferred aspect, the clinical signs, as mentioned herein, are selected from the group consisting of expelling of mummified, stillborn and/or weak fetuses.

In yet another preferred aspect, the clinical symptoms, as mentioned herein, are selected from the group consisting of, gross lesions, histologic lesions, replication of PCV3 in a tissue, and PCV3 viremia.

In still a further preferred aspect, the clinical symptoms, as mentioned herein, are selected from the group consisting of development or production of a mummified, stillborn and/or weak fetus.

The present disclosure also encompasses use of any of the herein disclosed compositions in any of the herein disclosed methods; or use of a PCV3 ORF2 protein, alone or in combination, of any one of the herein disclosed compositions, for use in the preparation of a composition for inducing an immunological or immune response or a protective immune or immunological response against (i) PCV3 and/or (ii) PCV2 and PCV3 and/or (iii) PCV3 and another porcine pathogen and/or (iv) PCV3, PCV2 and another porcine pathogen, or for use in a method for inducing an immunological or immune response or a protective immune or immunological response against (i) PCV3 and/or (ii) PCV2 and PCV3 and/or (iii) PCV3 and another porcine pathogen and/or (iv) PCV3, PCV2 and another porcine pathogen.

In one embodiment, the composition may be a PCV3 and PPV (advantageously packaged in a VLP) and/or PRRSV advantageously for use in breeding age sows/gilts. In such an embodiment, one or more doses for administration is contemplated. This particular embodiment encompasses use of a PCV3 ORF2 protein in combination with a PPV protein and optionally a PRRSV protein for use in the preparation of a composition for inducing an immunological or immune response or a protective immune or immunological response PCV3 and PPV and optionally PSSRV, or for use in a method for inducing an immunological or immune response or a protective immune or immunological response against PCV3 and PPV and optionally PSSRV.

In this embodiment, a composition may comprise a (i) porcine circovirus type 3 (PCV3) ORF2 protein, a parvovirus (PPV) protein and optionally a PRRSV (porcine respiratory and reproductive syndrome virus) protein and (ii) a veterinary-acceptable carrier selected from the group consisting of a solvent, a dispersion media, a coating, a stabilizing agent, a diluent, a preservative, an anti-microbial agent, an antifungal agent, an isotonic agent, an adsorption delaying agent, an adjuvant, cell culture supernatant, a stabilizing agent, a viral vector, an expression vector, an immunomodulatory agent, and/or any combination thereof. The veterinary-acceptable carrier may comprise an adjuvant, immunomodulatory agent, cell culture supernatant, viral or expression vector or any combination thereof. The veterinary-acceptable carrier may comprise an adjuvant. The composition may be utilized in a method for eliciting an immune response or an immunological response or a protective immune or immunological response against PCV3, PPV and/or PRRSV. In one embodiment, the composition may be utilized in a method for inducing an immune response against PCV3 in a pig, in particular in a preferably pregnant sow. In another embodiment, the composition may be utilized in a method of reducing or preventing the clinical signs or disease caused by an infection with a PCV3 in a piglet, wherein the piglet is to be suckled by a sow to which the composition has been administered. The composition may be administered intramuscularly or intradermally. The embodiment also relates to method for eliciting an immune response or an immunological response or a protective immune or immunological response against PCV3, PPV and/or PRRSV which may comprise administering to an animal any one of the above compositions. The embodiment also relates to method of immunizing swine against a clinical disease caused by at least one pathogen in said animal, said method comprising the step of administering to the animal any one of the above compositions, wherein said immunogenic composition fails to cause clinical signs of infection but is capable of inducing an immune response that immunizes the animal against pathogenic forms of said at least one pathogen.

PPV is an autonomous replicating virus of the Parvovirinae subfamily of the genus Protoparvovirus within the family Parvoviridae containing a single stranded DNA molecule of about 5100 nucleotides (Cotmore et al., 2014: Arch Virol.: 159(5): 1239-1247; Molitor et al., 1984: Virology: 137(2):241-54). Only the minus strand of the DNA is packaged into virions. The genome of the virus encodes three capsid proteins (VP1, VP2, VP3) and one non-structural protein (NS1). The capsid of parvovirus is about 22-25 nanometers in diameter and is comprised of VP1 and VP2 subunits. These proteins are derived from alternatively spliced versions of the same RNA molecule and thus overlap in sequence. Further, porcine parvovirus exhibits a high level of sequence similarity to feline panleukopenia virus, canine parvoviruses and rodent parvovirus (Ranz et al., 1989: J. gen. Virol: 70:2541-2553).

The PPV protein can be from an inactivated or killed whole cell or a subunit of PPV. Advantageously, the PPV protein is a recombinant PPV protein.

EP 0 551 449 A1 discloses a method for producing a VP2 subunit vaccine against porcine parvovirus. Cadar D et al. (Infection, Genetics and Evolution 2012, 12: 1163-1171) describe the phylogeny and evolutionary genetics of porcine parvovirus in wild boars. Streck A F et al. (Journal of General Virology 2011, 92: 2628-2636) describe the high rate of viral evolution in the capsid protein of porcine parvovirus. WO 88/02026 relates to empty viral capsid vaccines. Martinez C et al. (Vaccine 1992, 10(10): 684-690), discloses the production of porcine parvovirus empty capsids with high immunogenic activity. Xu F et al. (Applied and Environmental Microbiology 2007, 73(21): 7041-7047) describe the induction of immune responses in mice after intragastric administration of Lactobacillus casei producing porcine parvovirus VP2 protein. And U.S. Pat. No. 10,485,866 discloses immunogenic compositions comprising PPV viral protein 2 (VP2) advantageously a mutant PPV VP2 comprising one or more mutations.

The term “porcine parvovirus” or “PPV” is well known to the person skilled in the art. However, “Porcine parvovirus” is an autonomous replicating virus of the genus parvovirus within the family Parvoviridae containing a single stranded DNA molecule. The genome of the virus encodes three capsid proteins (VP1, VP2, VP3) and one non-structural protein (NS1). The disease caused by PPV in pigs is often referred to as a SMEDI (an acronym of stillbirth, mummification, embryonic death, and infertility). The term “porcine parvovirus” encompasses all possible strains, genotypes, phenotypes and serotypes of the porcine parvovirus. The term “viral protein 2” or “VP2” relates to the capsid protein VP2 of the porcine parvovirus. The term “viral protein 2” or “VP2” is well known to the person skilled in the art.

Porcine reproductive and respiratory syndrome (PRRS) is viewed by many as the most important disease currently affecting the pig industry worldwide. PRRS virus (PRRSV) is an enveloped single stranded RNA virus classified in the family Arteriviridae. There is large variability in the antigenic characteristics of the different isolates of PRRSV and effective measures to prevent infections are limited. There are three major groups of vaccines available for PRRS, attenuated modified live virus (MLV), killed virus vaccine or recombinant vaccines. The viral envelope proteins of PRRSV are generally categorized into major and minor proteins based on abundance of proteins in the virion. The major viral envelope proteins are gp5 (ORF 5) and M (ORF 6) and form a dimer. The minor envelope proteins are gp2 (ORF2), gp3 (ORF3), gp4 (ORF4) and E (ORF2b) and probably a newly identified viral protein gp5a (ORF 5a). The active antigenic component can include the ORF4, ORF5, ORF6, or ORF7 from PRRSV virus.

The recombinant PRRSV antigen may be expressed in a vectored PRRSV vaccine or composition that comprises one or more engineered, recombinant adenovirus vectors that harbor and express certain PRRSV antigens, and optionally a pharmaceutically or veterinarily acceptable carrier, adjuvant, excipient, or vehicle. Advantageous, the vector is an adenovirus vector although other vectors, such as a baculovirus, are also contemplated.

The PRRSV may be any strain, as the novel and inventive compositions and methods disclosed herein are universally applicable to all known and yet to be discovered PRRSV strains. PRRSV virus exists as two genotypes referred to as “US” and “EU” type which share about 50% sequence homology (Dea S et al. (2000). Arch Virol 145:659-88). These two genotypes can also be distinguished by their immunological properties. Most sequencing information on various isolates is based on the structural proteins, namely the envelope protein GP5 which accounts for only about 4% of the viral genome, while only little is known on the non-structural proteins (nsp). Isolation of PRRSV and manufacture of vaccines have been described in a number of publications (WO 92/21375, WO 93/06211, WO93/03760, WO 93/07898, WO 96/36356, EP 0 676 467, EP 0 732 340, EP 0 835 930, U.S. Pat. No. 10,039,821). The PRRSV antigen includes PRRSV minor proteins (e.g. gp2, gp3, gp4, gp5a, gp5 or E), in any combination, and optionally includes additional PRRSV major proteins (e.g. gp5 or M). For example, the PRRSV antigens could be displayed on the surface of virus-like particles (VLPs). In other embodiments, soluble versions of the antigens could be administered to the host animal, wherein oligomerization (including trimerization) of the proteins with each other, or additionally, with components of VSV-G, or other viral proteins or any oligomerization (including trimerization motifs) (e.g. motifs from bacterial GCN4, and the like). Moreover, the TM/CT domains of Type I viral surface glycoproteins are envisioned to accomplish the same purpose as, and are therefore interchangeable with, the corresponding domains from VSV-G.

In some embodiments, the one or more vectors comprise either: a nucleotide sequence encoding a PRRSV E antigen, polypeptide, ectodomain or variant thereof; or, a nucleotide sequence encoding a modified PRRSV gp2, gp3, gp4, gp5a, gp5 or M antigen, polypeptide, ectodomain, or variant thereof, wherein an existing cellular localization sequence of gp2, gp3, gp4, gp5a, gp5 or M has been replaced with a cell-surface expression determinant sequence from an heterologous gene. In some embodiments, the one or more vectors comprise a mixture of two vectors, a first vector expressing retargeted PRRSV minor proteins, and a second vector expressing re-targeted PRRSV major proteins

In an advantageous embodiment, the immunogenic composition comprising PCV3, PPV and/or PRRSV is administered in two doses to a subject of need. However, the immunogenic composition comprising PCV3, PPV and/or PRRSV may be administered at two or more doses, with a first dose being administered prior to the administration of a second (booster) dose. Preferably, the second dose is administered at least 15 days after the first dose. More preferably, the second dose is administered between 15 days and 40 days after the first dose. Even more preferably, the second dose is administered at least 17 days after the first dose. Still more preferably, the second dose is administered between 17 days and 30 days after the first dose. Even more preferably, the second dose is administered at least 19 days after the first dose. Still more preferably, the second dose is administered between 19 days and 25 days after the first dose. Most preferably the second dose is administered at least 21 days after the first dose. Even more preferably, the second dose is administered at about 21 days after the first dose or at 21 days after the first dose. In a preferred aspect of the two-time administration regimen, both the first and second doses of the immunogenic composition comprising PCV3, PPV and/or PRRSV are administered in the same amount. Preferably, each dose is in the preferred amounts specified above, with a dose of 1 ml or 2 ml for the first and second dose being most preferred. In addition to the first and second dose regimen, an alternate embodiment comprises further subsequent doses. For example, a third, fourth, or fifth dose could be administered in these aspects. Preferably, subsequent third, fourth, and fifth dose regimens are administered in the same amount as the first dose, with the time frame between the doses being consistent with the timing between the first and second doses mentioned above.

The dose volume per subject depends on the route of vaccination and the age of the subject. Preferably, the total volume is between about 0.2 ml and 5 ml, more preferably between about 0.5 ml and 3.0 ml, even more preferably between about 1.0 ml and 2.5 ml, even more preferably between about 1.0 ml and 2.0 ml. Most preferred the volume is 1 ml, 1.5 ml, 2 ml or 2.5 ml per dose.

The immunogenic composition comprising PCV3, PPV and/or PRRSV is, preferably, administered topically or systemically. Suitable routes of administration conventionally used are oral or parenteral administration, such as intranasal, intravenous, intradermal, transdermal, intramuscular, intraperitoneal, subcutaneous, as well as inhalation. However, depending on the nature and mode of action of a compound, the immunogenic composition may be administered by other routes as well. For example, such other routes include intracutaneously, intravenously, intravascularly, intraarterially, intraperitnoeally, intrathecally, intratracheally, intracutaneously, intracardially, intralobally, intralobarly, intramedullarly, intrapulmonarily, intrarectally, and intravaginally. However, more preferred the immunogenic composition comprising PCV3, PPV and/or PRRSV is administered subcutaneously or intramuscularly. Most preferred the immunogenic composition comprising PCV3, PPV and/or PRRSV is administered intramuscularly.

In one aspect, said immunogenic composition comprising PCV3, PPV and/or PRRSV is administered intramuscularly.

In one aspect, said immunogenic composition comprising PCV3, PPV and/or PRRSV is administered to gilts and/or sows.

Preferably, the immunogenic composition comprising PCV3, PPV and/or PRRSV is administered to gilts and/or sows being at least three 3 months of age, more preferably at least 4 months of age, most preferably at least 5 months of age.

In one aspect, the immunogenic composition is administered to gilts and/or sows being at least three 3 month of age.

In one aspect, said immunogenic composition comprising PCV3, PPV and/or PRRSV comprising PCV3, PPV and/or PRRSV is administered to gilts and/or sows before pregnancy.

In a two shot regime, the second dose of said immunogenic composition comprising PCV3, PPV and/or PRRSV is advantageously administered to gilts and/or sows 2, 3, 4 or 5 weeks before mating/insemination, most preferably about 3 weeks before mating/insemination. Preferably, the first dose of said immunogenic composition is administered to gilts and/or sows 2, 3, 4, 5 or 6 weeks before administering the second dose, most preferably about 3 weeks before administering the second dose. However, after the 2 shot regime has been applied, preferably, gilts and/or sows are revaccinated every 3, 4, 5, 6, 7 or 8 months, most preferably about every 6 months.

In one aspect of the present invention said immunogenic composition is administered to gilts and/or sows during pregnancy and lactation.

In one aspect of the present invention the immunogenic composition is safe for gilts and/or sows during pregnancy and lactation.

It is further claimed that, the vaccine is able to protect bred gilts and sows when challenged with PCV3 in all or two or at least one trimester during the 114 days of gestation.

It is also claimed that the vaccine is able to significantly reduce the incidence of mummies, stillborns and fetus in vaccinated gilts and sows vaccinated when challenged with PCV3 in all or two or at least one trimester during the 114 days of gestation.

In one aspect of the present invention the immunogenic composition is safe for gilts and/or sows from 30 days of gestation, preferably from 40 days of gestation.

Preferably, the immunogenic composition comprising PCV3, PPV and/or PRRSV comprises between 0.1 μg and 150 μg, preferably between 0.25 μg and 75 μg, more preferably between 0.5 μg and 37.5 μg, even more preferably between 0.5 μg and 15 μg, most preferably between 0.5 μg and 6 μg of the PCV3, PPV and/or PRRSV antigen. The immunogenic composition comprising PCV3, PPV and/or PRRSV can be in amounts of about 0.25 μg, 0.5 μg, 0.75 μg, 1 μg, 1.25 μg, 1.5 μg, 1.75 μg, 2 μg, 2.25 μg, 2.5 μg, 2.75 μg, 3 μg, 3.5 μg, 4 μg, 4.5 μg, 5 μg, 5.5 μg, 6 μg, 6.5 μg, 7 μg, 7.5 μg, 8 μg, 8.5 μg, 9 μg, 9.5 μg, 10 μg, 10.5 μg, 11 μg, 11.5 μg, 12 μg, 12.5 μg, 13 μg, 13.5 μg, 14 μg, 14.5 μg or 15 μg.

In one aspect of the present invention the immunogenic composition comprises between 0.1 μg and 150 μg of the PPV VP2 antigen, preferably between 0.5 μg and 30 μg of the immunogenic composition comprising PCV3, PPV and/or PRRSV antigens.

In one aspect, the immunogenic composition protects against a homologous and/or a heterologous challenge.

The PCV3 ORF2 protein may be produced by a baculovirus expression system in cultured insect cells. The method may include inactivating the baculovirus. Inactivation is conducted in a manner understood in the art. For example, in chemical inactivation, a suitable virus sample or serum sample containing the virus is treated for a sufficient length of time with a sufficient amount or concentration of inactivating agent at a sufficiently high (or low, depending on the inactivating agent) temperature or pH to inactivate the virus. Inactivation by heating is conducted at a temperature and for a length of time sufficient to inactivate the virus. Inactivation by irradiation is conducted using a wavelength of light or other energy source for a length of time sufficient to inactivate the virus. The virus is considered inactivated if it is unable to infect a cell susceptible to infection. The inactivating may comprise heat treatment or use of a virus inactivating agent. The inactivating agent may comprise an aziridine compound, such as BEI.

The present disclosure also includes a recombinant vector comprising a polynucleotide sequence that encodes a polypeptide sequence that encodes a PCV3 ORF2 protein. The PCV3 ORF2 may be from group a1, b1 or b2 (using the subtyping designation of Fux et al., “Full genome characterization of porcine circovirus type 3 isolates reveals the existence of two distinct groups of virus strains,” Virology Journal (2018) 15:25, DOI 10.1186/s12985-018-0929-3 (incorporated herein by reference); see, e.g., Table 4). In another embodiment, the PCV3 ORF2 protein comprises or consists of an amino acid sequence encoded by a polynucleotide sequence having at least 90%, or at least 91%, or at least 92%, or at least 93%, or at least 94%, or at least 95%, or at least 96%, or at least 97%, or at least 98%, or at least 99%, or 100% sequence identity or sequence homology with SEQ ID NO: 4. The recombinant vector may be a baculovirus. In another embodiment, the recombinant vector may comprise at least 90% or at least 91%, or at least 92%, or at least 93%, or at least 94%, or at least 95%, or at least 96%, or at least 97%, or at least 98%, or at least 99%, or 100% sequence identity or sequence homology with SEQ ID NO:2.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the disclosure.

The porcine, pig or piglet to which there is administration can have antibodies against a PCV, such as PCV2 and/or PCV3, e.g., maternal antibodies.

These and other embodiments are disclosed or are obvious from and encompassed by, the following Detailed Description.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following detailed description, given by way of example, but not intended to limit the invention solely to the specific embodiments described, may best be understood in conjunction with the accompanying drawings.

FIG. 1 is the sequence of the PCV3 ORF2 nucleotide sequence in recombinant baculovirus BaculoG/PCV3 ORF2, SEQ ID NO:1.

FIG. 2A-1 to FIG. 2H-3 is the sequence of the recombinant baculovirus BaculoG/PCV3 ORF2, SEQ ID NO:2.

FIG. 3 shows the map of the recombinant baculovirus containing the PCV3 ORF2 gene under control of the baculovirus polyhedrin promoter (BaculoG/PCV3 ORF2 Clone 4B4-2E12 Pre-MSV p8).

FIG. 4 shows group median log 10 PCV3 DNA genomic copies/mL in serum by study day; Groups 1-5.

FIG. 5 shows group median log 10 PCV3 DNA genomic copies/mL by study day in fecal samples; Groups 1-5.

FIG. 6 shows group median log 10 PCV3 DNA genomic copies/mL by study day in nasal samples; Groups 1-5.

FIG. 7 shows baseline adjusted, least square group mean rectal temperatures (° F.) by study day.

FIG. 8 shows baseline-adjusted, group least square means daily weight (kg) by day; Groups 1-5.

FIG. 9 shows group mean body temperatures (° F.) by day.

FIG. 10A-C shows sequence information on the PCV3 PCR positive tissue homogenate used for challenge material (SEQ ID NOs: 3-5).

FIG. 11 shows the median PCR value for Groups 1-5 from seven to forty-nine days.

FIG. 12 shows the median PCR value for Groups 7-9 from seven to forty-nine.

FIG. 13 shows the median PCR values for fecal shedding for Groups 1-5 from seven to forty-nine days.

FIG. 14 shows the median PCR values for nasal shedding for Groups 1-5 from seven to forty-nine days.

FIG. 15 depicts the arithmetic mean rectal temperature value for Groups 1-6 from fourteen to forty-nine days of the evaluation.

FIG. 16 depicts the least-squares mean temperature values by group and day for groups 1-5.

FIG. 17 is a line chart illustrating the mean rectal temperatures of animals (Baseline Adjusted Least-Squares) by Group and Day for Groups 1-5.

FIG. 18 shows the arithmetic mean body weight values for Groups 1-6 from fourteen to forty-nine days.

FIG. 19 is a line graph showing the body weight (Least-Squares) means by Group and Day for Groups 1-5.

FIG. 20 is a line graph depicting data for Least-Squares Means for body weight (Baseline Adjusted) by Group and day.

FIG. 21 shows a history plot of pre-MSV+1 production.

FIG. 22A shows cell count and FIG. 22B shows cell viability and size during infection with BaculoG/PCV3 ORF2.

FIG. 23 shows an analysis of BaculoG/PCV3 ORF2 fluids at harvest.

FIG. 24 shows images of inactivations at 72 hours.

FIG. 25 shows western comparison of inactivation conditions for BaculoG/PCV3 ORF2 antigen—post inactivation.

FIG. 26 shows a PCV3 ORF2 fluorescent dot blot.

FIG. 27 shows a plot of observed viremia in the sample population of pigs post-challenge based on the log 10 genomic copies/mL. All control pigs were viremic as determined by PCR at each sampling point during the challenge phase, and the viral load at each sampling point during the challenge phase was significantly reduced by vaccination (P≤0.0050).

FIG. 28 shows a plot of the measured mean rectal temperatures (° F.) pre-challenge (D12, D13, D14) and post-challenge (D14.5-D20).

FIG. 29 shows a plot of the observed viremia in gilts challenged at D83 (40 days into gestation). Numbers indicate genomic copies/mL. The Y-axis is shown on a linear scale to accurately represent values at zero. Arrows indicate administration of primary vaccine, booster and challenge.

FIG. 30 shows a bar graph indicating the percent of affected piglets based on the observed number of autolyzed, crushed, mummified born piglets from farrowing sow of each treatment group.

FIG. 31A-C shows the alignment of the amino acid sequence of the PCV3 capsid with the capsid of porcine PCV2 and the capsid of beak and feather disease virus (BFDV). FIG. 31 discloses SEQ ID NOS 22-33, respectively, in order of appearance.

FIG. 32 shows the structure of the PCV3 ORF2 mutant in the FG loop having mutations in the lysines and histidines.

FIG. 33 shows the structure of the PCV3 ORF2 mutant wherein the native stop codon for the PCV3 capsid protein was mutated and the C-terminus was extended to the next stop codon. FIG. 33 discloses SEQ ID NOS 14, 17-19, 11-12, and 34-37, respectively, in order of appearance.

FIG. 34 depicts the nucleotide and amino acid sequences of a PCV3 ORF2 mutant in the FG loop having mutations in the lysines and histidines and a PCV3 ORF2 mutant wherein the native stop codon for the PCV3 capsid protein was mutated and the C-terminus was extended to the next stop codon (SEQ ID NOs: 6-9).

FIG. 35 depicts the amino acid sequence of Mutated PCV3 ORF2 “FG-PC” (SEQ ID NO: 10).

### DETAILED DESCRIPTION OF THE INVENTION

The present disclosure relates to a PCV3 vaccine.

Any sequence of PCV3 is contemplated. See, eg., Phan, Tung Gia, et al. “Detection of a Novel Circovirus PCV3 in Pigs with Cardiac and Multi-Systemic Inflammation.” Virology Journal, vol. 13, no. 1, 2016, p. 184, doi: 10.1186/s12985-016-0642-z. Published Nov. 11, 2016 and Fux et al., “Full genome characterization of porcine circovirus type 3 isolates reveals the existence of two distinct groups of virus strains,” Virology Journal (2018) 15:25, DOI 10.1186/s12985-018-0929-3 the disclosures of which are incorporated by reference.

The PCV3 ORF2 and the PCV3 genome sequences were derived from KT869077 (GenBank). Whole PCV3 genome in a plasmid was used and described in the Examples. ORF2 and whole genome were synthesized at Genscript.

Two additional constructs, re-circularized PCV3 genome derived by two different methods, were used in cell culture to rescue the virus.

The following sequences are presented in the sequence listing:

| SEQ | | |
|---|---|---|
| ID NO: | Type | Description |
| 1 | DNA | Polynucleotide encoding PCV3 ORF2 from |
| | | baculovirus vector |
| 2 | DNA | Polynucleotide encoding PCV3 ORF2 in baculovirus |
| | | vector |
| 3 | Protein | PCV3 ORF1 isolated from tissue |
| 4 | Protein | PCV3 ORF2 isolated from tissue |
| 5 | DNA | Polynucleotide encoding PCV3 ORF2 isolated from |
| | | tissue |
| 6 | DNA | Polynucleotide encoding mutated PCV3 ORF2 “FG” |
| 7 | DNA | Polynucleotide encoding mutated PCV3 ORF2 “PC” |
| 8 | Protein | Mutated PCV3 ORF2 “FG” |
| 9 | Protein | Mutated PCV3 ORF2 “PC” |
| 10 | Protein | Mutated PCV3 ORF2 “FG-PC” |
| 11 | Protein | Portion of PCV3 ORF2 protein FG Loop |
| 12 | Protein | Replacement portion of PCV3 ORF2 protein FG Loop |
| 13 | Protein | PCV2 ORF2 (capsid) protein epitope |
| 14 | Protein | substitution in the FG loop of SEQ ID NO. 1 |
| 15 | Protein | substitution in the FG loop of SEQ ID NO. 1 |
| 16 | Protein | PCV2 ORF2 (capsid) protein epitope |
| 17 | Protein | Replacement portion of PCV3 ORF2 protein FG Loop |
| 18 | Protein | Replacement portion of PCV3 ORF2 protein FG Loop |
| 19 | Protein | Replacement portion of PCV3 ORF2 protein FG Loop |
| 20 | Protein | C-terminal extension of PCV3 ORF2 protein |
| 21 | Protein | C-terminal extension of PCV3 ORF2 protein |
| 22-33 | DNA | aa alignment of capsids of PCV3, PCV2 and BFVD |
| 34-37 | Protein | PCV3 ORF2 mutations in the lysines and histidines |
| | | of the FG loop |

PCV3 ORF2 “FG” is an antigenic protein according to the present invention that comprises amino acid substitutions in the FG loop of the natural PCV3 ORF2 protein.

PCV3 ORF2 “PC” is an antigenic protein according to the present invention that comprises an amino acid extension at the C terminal end of the natural PCV3 ORF2 protein.

In a preferred aspect, the polypeptide of the present disclosure is a recombinant PCV3 ORF2 protein, such as a recombinant baculovirus expressed PCV3 ORF2 protein. The term “recombinant PCV3 ORF2 protein”, as used herein, in particular refers to a protein molecule which is expressed from a recombinant DNA molecule, such as a polypeptide, which is produced by recombinant DNA techniques. An example of such techniques includes the case when DNA encoding the expressed protein is inserted into a suitable expression vector, preferably a baculovirus expression vector, which is in turn used to transfect, or in case of a baculovirus expression vector to infect, a host cell to produce the protein or polypeptide encoded by the DNA. The term “recombinant PCV3 ORF2 protein”, as used herein, thus in particular refers to a protein molecule, which is expressed from a recombinant DNA molecule.

According to a particular example, the recombinant PCV3 ORF2 protein is produced by a method with the following steps: The gene for PCV3 ORF2 is cloned into a baculovirus transfer vector; the transfer vector is used to prepare recombinant baculovirus containing said gene by homologous recombination in insect cells; and the PCV3 ORF2 protein is then expressed in insect cells during infection with the recombinant baculovirus.

It is further understood that the term “recombinant PCV3 protein consisting of a sequence” in particular also concerns any cotranslational and/or posttranslational modification or modifications of the sequence affected by the cell in which the polypeptide is expressed. Thus, the term “recombinant PCV3 ORF2 protein consisting of a sequence”, as described herein, is also directed to the sequence having one or more modifications effected by the cell in which the polypeptide is expressed, in particular modifications of amino acid residues effected in the protein biosynthesis and/or protein processing, preferably selected from the group consisting of glycosylations, phosphorylations, and acetylations.

Preferably, the recombinant PCV3 ORF2 protein according to the disclosure is produced or obtainable by a baculovirus expression system, in particular in cultured insect cells.

In yet a further preferred aspect, the polypeptide of the present disclosure is a PCV3 ORF2 protein comprising or consisting of an amino acid sequence having at least 90%, preferably at least 92%, more preferably at least 94%, even more preferably at least 96%, still more preferably at least 98%, or in particular 100% sequence identity with the amino acid sequence of SEQ ID NO: 4.

“Sequence Identity” as it is known in the art refers to a relationship between two or more polypeptide sequences or two or more polynucleotide sequences, namely a reference sequence and a given sequence to be compared with the reference sequence. Sequence identity is determined by comparing the given sequence to the reference sequence after the sequences have been optimally aligned to produce the highest degree of sequence similarity, as determined by the match between strings of such sequences. Upon such alignment, sequence identity is ascertained on a position-by-position basis, e.g., the sequences are “identical” at a particular position if at that position, the nucleotides or amino acid residues are identical. The total number of such position identities is then divided by the total number of nucleotides or residues in the reference sequence to give % sequence identity. Sequence identity can be readily calculated by known methods, including but not limited to, those described in Computational Molecular Biology, Lesk, A. N., ed., Oxford University Press, New York (1988), Biocomputing: Informatics and Genome Projects, Smith, D. W., ed., Academic Press, New York (1993); Computer Analysis of Sequence Data, Part I, Griffin, A. M., and Griffin, H. G., eds., Humana Press, New Jersey (1994); Sequence Analysis in Molecular Biology, von Heinge, G., Academic Press (1987); Sequence Analysis Primer, Gribskov, M. and Devereux, J., eds., M. Stockton Press, New York (1991); and Carillo, H., and Lipman, D., SIAM J. Applied Math., 48: 1073 (1988), the teachings of which are incorporated herein by reference. Preferred methods to determine the sequence identity are designed to give the largest match between the sequences tested. Methods to determine sequence identity are codified in publicly available computer programs which determine sequence identity between given sequences. Examples of such programs include, but are not limited to, the GCG program package (Devereux, J., et al., Nucleic Acids Research, 12(1):387 (1984)), BLASTP, BLASTN and FASTA (Altschul, S. F. et al., J. Molec. Biol., 215:403-410 (1990). The BLASTX program is publicly available from NCBI and other sources (BLAST Manual, Altschul, S. et al., NCVI NLM NIH Bethesda, Md. 20894, Altschul, S. F. et al., J. Molec. Biol., 215:403-410 (1990), the teachings of which are incorporated herein by reference). These programs optimally align sequences using default gap weights in order to produce the highest level of sequence identity between the given and reference sequences. As an illustration, by a polynucleotide having a nucleotide sequence having at least, for example, 85%, preferably 90%, even more preferably 95% “sequence identity” to a reference nucleotide sequence, it is intended that the nucleotide sequence of the given polynucleotide is identical to the reference sequence except that the given polynucleotide sequence may include up to 15, preferably up to 10, even more preferably up to 5 point mutations per each 100 nucleotides of the reference nucleotide sequence. In other words, in a polynucleotide having a nucleotide sequence having at least 85%, preferably 90%, even more preferably 95% identity relative to the reference nucleotide sequence, up to 15%, preferably 10%, even more preferably 5% of the nucleotides in the reference sequence may be deleted or substituted with another nucleotide, or a number of nucleotides up to 15%, preferably 10%, even more preferably 5% of the total nucleotides in the reference sequence may be inserted into the reference sequence. These mutations of the reference sequence may occur at the 5′ or 3′ terminal positions of the reference nucleotide sequence or anywhere between those terminal positions, interspersed cither individually among nucleotides in the reference sequence or in one or more contiguous groups within the reference sequence. Analogously, by a polypeptide having a given amino acid sequence having at least, for example, 85%, preferably 90%, even more preferably 95% sequence identity to a reference amino acid sequence, it is intended that the given amino acid sequence of the polypeptide is identical to the reference sequence except that the given polypeptide sequence may include up to 15, preferably up to 10, even more preferably up to 5amino acid alterations per each 100 amino acids of the reference amino acid sequence. In other words, to obtain a given polypeptide sequence having at least 85%, preferably 90%, even more preferably 95% sequence identity with a reference amino acid sequence, up to 15%, preferably up to 10%, even more preferably up to 5% of the amino acid residues in the reference sequence may be deleted or substituted with another amino acid, or a number of amino acids up to 15%, preferably up to 10%, even more preferably up to 5% of the total number of amino acid residues in the reference sequence may be inserted into the reference sequence. These alterations of the reference sequence may occur at the amino or the carboxy terminal positions of the reference amino acid sequence or anywhere between those terminal positions, interspersed either individually among residues in the reference sequence or in the one or more contiguous groups within the reference sequence. Preferably, residue positions which are not identical differ by conservative amino acid substitutions. However, conservative substitutions are not included as a match when determining sequence identity.

“Sequence homology”, as used herein, refers to a method of determining the relatedness of two sequences. To determine sequence homology, two or more sequences are optimally aligned, and gaps are introduced if necessary. However, in contrast to “sequence identity”, conservative amino acid substitutions are counted as a match when determining sequence homology. In other words, to obtain a polypeptide or polynucleotide having 95% sequence homology with a reference sequence, 85%, preferably 90%, even more preferably 95% of the amino acid residues or nucleotides in the reference sequence must match or comprise a conservative substitution with another amino acid or nucleotide, or a number of amino acids or nucleotides up to 15%, preferably up to 10%, even more preferably up to 5% of the total amino acid residues or nucleotides, not including conservative substitutions, in the reference sequence may be inserted into the reference sequence. Preferably the homologous sequence comprises at least a stretch of 50, even more preferably 100, even more preferably 250, even more preferably 500 nucleotides.

A “conservative substitution” refers to the substitution of an amino acid residue or nucleotide with another amino acid residue or nucleotide having similar characteristics or properties including size, hydrophobicity, etc., such that the overall functionality does not change significantly.

The present invention also encompasses mutations of PCV3 proteins, such as but not limited to mutations of the PCV3 capsid protein. Despite the divergence of the capsid amino acid sequences between PCV2 and beak and feather disease virus (BFDV), the crystal structures are very similar despite their sequence divergence. Advantageously, the mutations of PCV3 are to stabilize virus-like particles (VLPs). The PCV3 capsid protein should self-assemble into a VLP, however, the level of expression of the PCV3 protein is significantly lower as compared to the PCV2 capsid protein. Specifically, only about 20% of the protein assembles into VLPs whereas the remaining 80% of the protein aggregates into an insoluble fraction.

In some embodiments, the variant protein of the present invention is capable of a higher yield of VLPs than the protein encoded by SEQ ID No. 1. It is understood that higher yield in particular—and for example—relates to higher molar yield. Alternatively expressed, the variant protein of the present invention is capable of a larger assembly of CAP (capsid (ORF2) protein) VLPs than the protein encoded by SEQ ID No. 1. Examples of higher yields include at least 5% higher yield, or at least 10% higher yield, or at least 15% higher yield, or at least 20% higher yield, or at least 25% higher yield, or at least 30% higher yield, or at least 35% higher yield, or at least 40% higher yield, or at least 50% higher yield. Thus, for example, if without a modification of the PCV3 ORF2 protein, by baculovirus expression, there is 20% PCV3 soluble protein (VLP) and 80% PCV3 insoluble protein, e.g., by Western Blot, and by the modification there is, instead, 25%, or 30%, or 35%, or 40%, or 45%, or 50%, or 55%, or 60% or higher PCV3 soluble protein (VLP) (whereby there has been an increase of 5% or 10%, or 15%, or 20%, or 25%, or 30%, or 35%, or 40%, or 45%, etc. of PCV3 soluble protein (VLP)), that represents a higher yield. Advantageously, from modifying the PCV3 ORF2 protein, the VLP yield (soluble PCV3 proteins) is at least 50% of the PCV3 proteins expressed by the recombinant baculovirus system.

Assays and techniques suitable for use in the present invention include those that have been used for the tracking or quantifying the assembly and disassembly of porcine circovirus capsid (ORF2) protein into virus-like particles (VLPs) and these include: enzyme-linked immunosorbent assay (ELISA), SDS/PAGE optionally with silver stain or coomassie stain, western blot or immunoblot, size exclusion chromatography (SEC), dynamic light scattering (DLS) or multi-angled light scattering (MALS), transmission electron microscopy (TEM), analytical ultracentrifugation, and fluorescence spectroscopic analysis (FSA) optionally coupled with high performance liquid chromatography (HPLC). Additional suitable techniques may also include: agarose gel retardation tests of protein-nucleic acid complexes, immune diffusion tests e.g. single radial immunodiffusion (SRID), nanoparticle tracking analysis (NTA), metabolic labelling and chemiluminescent enzyme-based assays. Each of these assays is well-known in the art and is described in, for example, Fang, Mingli et al. “Detection of the Assembly and Disassembly of PCV2b Virus-Like Particles Using Fluorescence Spectroscopy Analysis” Intervirology vol. 58, 2015, pp. 318-323; Thompson, Christine et al. “Analytical technologies for influenza virus-like particle candidate vaccines: challenges and emerging approaches” Virology Journal vol 10, 2013, p. 141; Steppert, Petra et al. “Quantification and characterization of virus-like particles by size-exclusion chromatography and nanoparticle tracking analysis” Journal of Chromatography A vol. 1487, 2017, pp. 89-99; Yadav, Shalini et al. “A facile quantitative assay for viral particle genesis reveals cooperativity in virion assembly and saturation of an antiviral protein” Virology, vol 429, No. 2, 2012, pp. 155-162; and Zeltins, Andris “Construction and Characterization of Virus-Like Particles: A Review” Molecular Biotechnology vol. 53, 2013, pp. 92-107, each of which is incorporated herein by reference in its entirety.

In one aspect, the variant protein of the present invention is capable of a higher yield of VLPs than the protein encoded by SEQ ID No. 1 as determinable by Western blot analysis. In other words, the variant protein of the present invention is capable of a larger assembly of CAP VLPs than the protein encoded by SEQ ID No. 1 as determinable by Western blot analysis.

In the various embodiments discussed herein wherein there is mutation or mutations of the PCV3 ORF2 capsid protein, e.g., to increase VLP yield. For example, in various embodiments there can be one, two, three, or four mutations in the FG loop. Exemplified and discussed herein are embodiments that may involve the SKKK (SEQ ID NO: 11) of the PCV3 ORF2 protein FG Loop replaced with QPFS (SEQ ID NO: 12) (e.g., a PCV2 ORF2 protein motif). In making the substitution(s), the skilled artisan can practice the invention by only replacing the S with Q or only replacing the first K with P or only replacing the second K with F or only replacing the third K with S, or any combinations of these replacements, e.g., S to Q and first K to P or S to Q and second K to F or S to Q and third K to S, or S to Q and first K to P and second K to F, or S to Q and first K to P and third K to S, etc. Likewise, in these embodiments, in addition to or as an alternative to the replacement(s) or mutation(s) in the FG loop, the skilled artisan can practice the invention by adding amino acids to the C-terminus of the PCV3 ORF2 protein. Without an extension or addition to the C-terminus, the PCV3 ORF2 protein may be, in the three-dimensional structure, buried, versus exposed as are the C-terminus of other circovirus ORF2 or capsid proteins. In embodiments where there is extension or addition of the C-terminus of the PCV3 ORF2 protein, it may be advantageous to extend or add to the C-terminus of the PCV3 ORF2 protein with a motif from another circovirus, such as, for example, PCV2. Thus, for instance, one skilled in the art can extend or add to the C-terminus of the PCV3 ORF2 protein with amino acids found at the C-terminus of a PCV2 ORF2 protein or capsid protein, such as amino acids 215-234 or 215-233 of a PCV2 ORF2 protein or capsid protein. The skilled artisan can extend or add to the PCV3 ORF2 protein or capsid protein with epitope(s) of a PCV2 ORF2 protein or capsid protein. In this regard, mention is made of Trible et al., “Antibody Recognition of Porcine Circovirus Type 2 Capsid Protein Epitopes after Vaccination, Infection and Disease, Clinical and Vaccine Immunology 18(5): 749-757 (2011) doi:10.1128/CVI.00418-10 (incorporated herein by reference). In PCV2 ORF2 (capsid) protein immunoreactive regions are reported between residues 47 and 85, 165 and 200, and 200 and 233. Antibody reactive regions of PCV2 ORF2 (capsid) protein are reported as between amino acids 23 and 43, 71 and 85, 117 and 131, and 171 and 202. The PCV2 ORF2 (capsid) protein region of amino acids 117 to 131 is reported as a dominant antibody recognition region, and amino acids 156 to 162, 175 to 192, 195 to 202 and 228 to 223 are reported as associated with antibody recognition. Another PCV2 ORF2 (capsid) protein epitope is 169-STIDYFQPNNKR (SEQ ID NO: 13), e.g., amino acids 169-180 (wherein Y-173, F-174, Q-175, and K-179 amino acid residues may contribute to antibody recognition). Other PCV2 ORF2 (capsid) protein epitopes can be amino acids 43-233, 43-135, 43-160, 91-160, 43-180, 160-233, 135-233 and 91-233, as well as amino acids 169-188. Any of these, or any combination of these PCV2 ORF2 epitope(s) can be the C-terminus extension or addition to the PCV3 ORF2 (capsid) protein. In this regard, it is mentioned that the C-terminus extension of PCV3 ORF2 can be up to about 200 amino acids, or up to about 190 amino acids, or up to about 185 amino acids, or up to about 180 amino acids, or up to about 175 amino acids, or up to about 170 amino acids or up to about 165 amino acids, or up to about 160 amino acids or up to about 155 amino acids, or up to about 150 amino acids, or up to about 145 amino acids, or up to about 140 amino acids, or up to about 135 amino acids, or up to about 130 amino acids, or up to about 125 amino acids, or up to about 120 amino acids, or up to about 115 amino acids, or up to about 110 amino acids, or up to about 105 amino acids, or up to about 100 amino acids, up to about 90 amino acids, or up to about 80 amino acids or up to about 70 amino acids, or up to about 60 amino acids, or up to about 50 amino acids, or up to about 40 amino acids, or up to about 30 amino acids, in length; for instance, from 1-50 amino acids or 10-50 amino acids or 10-40 amino acids or 20 to 40 amino acids or about 30 amino acids in length.

In embodiments where a composition contains a PCV3 ORF2 (capsid) protein of the invention, e.g., such a protein that has been mutated, e.g., wherein the mutation includes addition or extension of the C-terminus, e.g., wherein the addition or extension of the C-terminus comprises epitope(s) of PCV2 ORF2 (capsid) protein, and the composition also includes a PCV2 ORF2 (capsid) protein (e.g., for a one-shot administration against both PCV2 and PCV3 or indications or symptoms or conditions thereof, e.g., each from baculovirus expression, e.g., alone or with one or more antigen of a porcine pathogen, such as those antigen(s) or porcine pathogen(s) disclosed throughout this disclosure), it may be advantageous that the PCV2 ORF2 (capsid) protein epitope(s) be of a clade that is the same as or different than that of the PCV2 ORF2 (capsid) protein included in the composition. For example, if the PCV2 ORF2 (capsid) protein component is from PCV2a strains (as Ingelvac CircoFlex may be based upon), it may be advantageous for the addition or extension on the PCV3 ORF2 capsid protein (C-terminus) to be from a different clade, e.g., a PCV2b, PCV2c, or PCVd-mPCV2b genotype. With respect to PCV2 genotypes or strains or clade, mention is made of Franzo et al., “Revisiting the taxonomical classification of Porcine Circovirus type 2 (PCV2): still a real challenge,” Virol J 12: 131 (2015) doi: 10.1186/s12985-015-0361-x (incorporated herein by reference). It may be advantageous that the PCV3 ORF2 capsid protein C-terminus addition or extension be of the same clade, strain or genotype as that of the PCV2 ORF2 capsid protein component of the composition, or a different clade, strain or genotype, but is an epitope of a PCV2 ORF2 capsid protein that provides an immunological response against one or more of the PCV2 clades, strains or genotypes. With respect to the foregoing, and more generally, the mutated PCV3 ORF2 capsid proteins of the invention discussed throughout this disclosure, the invention comprehends nucleic acid molecules encoding such mutated PCV3 ORF2 capsid proteins, vectors, such as baculovirus vectors (see EP 2 460 821 A2, incorporated herein by reference, along with the documents cited therein as the methods and materials therein for expressing PCV2 ORF2 capsid protein via a baculovirus expression system can be employed in the practice of the present invention to express PCV3 ORF2 capsid protein, including such mutated proteins as herein disclosed, as well as a PCV2 ORF2 capsid protein, if desired to include such in a composition of the invention), containing such nucleic acid molecules, and methods for producing or expressing such mutated PCV3 ORF2 capsid proteins of the invention, such as by infecting or transfecting relevant cells with the vector (e.g., if the vector be baculovirus, a relevant cell can be an insect or Sf cell or Sf+ cell; see EP 2 460 821 A2, incorporated herein by reference, along with the documents cited therein). It is advantageous to recover or isolate the protein after expression or production, e.g., separating solids and retaining liquid or supernatant that contains soluble protein (e.g., VLPs). Compositions as discussed in this paragraph as well as throughout this disclosure can contain mutated PCV3 ORF2 capsid protein (and optionally additionally PCV2 ORF2 capsid protein and/or one or more additional antigen of a porcine pathogen), in amounts as discussed throughout this disclosure, and can be administered in regimen(s) as discussed throughout this disclosure, such as in a one-shot, or single dose, administration, and can be so administered to pigs or piglets as discussed throughout this disclosure.

In the context of the invention, the protein of the present invention as the antigen in the composition, such as the immunological composition, prevents or treats a PCV3 infection-associated disease or condition in a subject by for example inducing, stimulating or enhancing the immune response against PCV3.

Previous studies have shown that expressing the full-length PCV3 cap gene and NLS domains presenting within the N-terminal arginine rich motif (ARM) may cause misfolding of the protein and induce formation of circular virus complexes of 10-12 nm (Sarker et al. Nat Commun. 2016 Oct. 4; 7( ) 13014). Wang et al. (AMB Expr 10, 3 (2020) doi.org/10.1186/s13568-019-0940-0) reported the ability of PCV3 VLPs to self-assemble which were successfully expressed in E. coli and applied in the development of an ELISA for testing the specific antibodies of clinical pig serum. Specifically, to achieve high-level expression of recombinant PCV3 Cap in E. coli, the gene of wild-type entire Cap (wt-eCap) was amplified from clinical samples, and three optimized entire Cap (opti-eCap) and one optimized Cap deleted nuclear location signal (NLS) (opti-dCap) gene fragments encoding the same amino acid sequence with wt-eCap were synthesized based on the codon bias of E. coli. Unlike the present invention, regions beside the NLS of the PCV3 capsid have not been targeted with respect to VLP assembly and/or stability. Furthermore, removal of the NLS does not necessarily result in improved VLP assembly. However, embodiments of the invention can include removal or alteration of the PCV3 ORF2 capsid protein NLS, e.g., in addition to one or more of the FG loop mutations and/or C-terminus extension(s) discussed herein.

In an advantageous embodiment, the present invention encompasses mutating regions encoding positively charged amino acids in PCV proteins, such as but not limited to a PCV3 capsid protein. In particular, PCV3 capsid contains large amounts of positive charge in the FG loop, which sits at the 5-fold interface of the PCV3 capsid. The large amount of positive charge in this region may result in repulsive forces without the presence of nucleic acid, as would be expected of VLPs. In one embodiment of the invention, the positively charged amino acids are mutated to neutral and/or negative charged amino acids. In an advantageous embodiment, the lysines and histidine in this loop are mutated to the amino acids from PCV2 capsid (SEQ ID NO: 6).

In an embodiment, the invention provides an engineered PCV3 ORF2 protein comprising reduced amounts of positive charged amino acids as compared to a non-engineered PCV3 ORF2 protein. The non-engineered protein can be a wild-type or naturally occurring PCV3 ORF2 protein or can be an ORF2 protein already modified for another purpose for which it is desired to improve capsid formation activity, such as improved self-assembly in the presence or absence of a packageable polynucleotide.

In an embodiment, one or more positively charged amino acids are substituted, such as one or more lysine, arginine, or histidine, or combination thereof. In an embodiment, two or more positively charged amino acids are substituted. In an embodiment, three or more positively charged amino acids are substituted. In certain embodiments, charge associated with a region of the ORF2 protein, such as but not limited to the FG loop, is made more negative by substituting in one or more negatively charged amino acids. In certain embodiments, positively charged amino acids are substituted by amino acids that are less positively charged, and/or non-positively charged amino acids are substituted by amino acids more negatively charged. That is, the charge of a region of ORF2 can be made by altered by removing positive charge, adding negative charge, or both.

In an advantageous embodiment, the present invention encompasses adding additional amino acids to PCV proteins, such as but not limited to a PCV3 capsid protein. The short hydrophobic nature of the PCV3 capsid C-terminus would lead to the C-terminus being buried in the capsid and could lead to VLP instability without the presence of nucleic acid. In contrast, the C-terminus of PCV2 and BFDV capsid proteins project out away from the capsid. In one embodiment, the C-terminus of the PCV3 capsid is extended by about 1 to 50 amino acids, about 10 to 40, amino acids, or about 20 to 30 amino acids. In another embodiment, the C-terminus of the PCV3 capsid is extended by about 20, about 21, about 22, about 23, about 24, about 25, about 26, about 27, about 28, about 29, about 30, about 31, about 32, about 33, about 34, about 35, about 36, about 37, about 38, about 39 or about 40 amino acids. In an advantageous embodiment, the C-terminus of the PCV3 capsid protein is extended by mutating the stop codon. In a particularly advantageous embodiment, the native stop codon for the PCV3 capsid protein is mutated and the C-terminus was extended to the next stop codon in the virus sequence (SEQ ID NO: 7). In another embodiment, the C-terminus of the PCV capsid may be extended and/or swapped out with the C-terminus of other porcine circoviruses. The C-terminus of the PCV3 capsid protein may be extended about 50 to about 200 amino acids, about 60 to about 190 amino acids, about 70 to about 180 amino acids, about 80 to about 170 amino acids, about 90 to about 160 amino acids or about 100 to about 150 amino acids.

In certain embodiments, C-terminal extension comprises addition of amino acids at the C-terminus of a PCV3 capsid, for example by mutation of a stop codon. A stop codon can be mutated by deletion, substitution or insertion. In certain embodiments, C-terminal extension comprise insertion of amino acids near the C-terminus, including but not limited to insertion of amino acids one residue from the C-terminus, or two residues from the C-terminus, or three residues, or four residues, or five residues, or six, or seven, or eight, or more residues upstream form the C-terminus. In one embodiment, the residues may be any set of negatively charged amin acids.

It should be understood that the proteins of the invention may differ from the exact sequences illustrated and described herein. Thus, the invention contemplates deletions, additions and substitutions to the sequences shown, so long as the sequences function in accordance with the methods of the invention. In this regard, particularly preferred substitutions will generally be conservative in nature, i.e., those substitutions that take place within a family of amino acids. For example, amino acids are generally divided into four families: (1) acidic—aspartate and glutamate; (2) basic—lysine, arginine, histidine; (3) non-polar—alanine, valine, leucine, isoleucine, proline, phenylalanine, methionine, tryptophan; and (4) uncharged polar—glycine, asparagine, glutamine, cysteine, serine threonine, tyrosine. Phenylalanine, tryptophan, and tyrosine are sometimes classified as aromatic amino acids. It is reasonably predictable that an isolated or non-naturally occurring replacement of leucine with isoleucine or valine, or vice versa; an aspartate with a glutamate or vice versa; a threonine with a serine or vice versa; or a similar conservative replacement of an amino acid with a structurally related amino acid, will not have a major effect on the biological activity. Proteins having substantially the same amino acid sequence as the sequences illustrated and described but possessing minor amino acid substitutions that do not substantially affect the immunogenicity of the protein are, therefore, within the scope of the invention.

The invention further encompasses nucleotide sequences encoding functionally and/or antigenically equivalent variants and derivatives of the antigens of the invention and functionally equivalent fragments thereof. These functionally equivalent variants, derivatives, and fragments display the ability to retain antigenic activity. For instance, changes in a DNA sequence that do not change the encoded amino acid sequence, as well as those that result in conservative substitutions of amino acid residues, one or a few amino acid deletions or additions, and substitution of amino acid residues by amino acid analogs are those which will not significantly affect properties of the encoded polypeptide. Conservative amino acid substitutions are glycine/alanine; valine/isoleucine/leucine; asparagine/glutamine; aspartic acid/glutamic acid; serine/threonine/methionine; lysine/arginine; and phenylalanine/tyrosine/tryptophan. In one embodiment, the variants have at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% homology or identity to the antigen, epitope, immunogen, peptide or polypeptide of interest.

In some embodiments, the substitution introduces a conservative change, which replaces the amino acid with another amino acid of similar chemical structure, similar chemical properties or similar side-chain volume. The amino acids introduced may have similar polarity, hydrophilicity or hydrophobicity to the amino acids they replace. Conservative amino acid changes are well known in the art. Where amino acids have similar polarity, this can also be determined by reference to the hydropathy scale for amino acid side chains.

Conservative amino acid changes may also be determined by reference to the Point Accepted Mutation (PAM) or BLOcks Substitution Matrix (BLOSUM) family of scoring matrices for conservation of amino acid sequence. Thus, conservative amino acid changes may be members of an equivalence group, being a set of amino acids having mutually positive scores in the similarity representation of the scoring matrix selected for use in an alignment of the reference and mutant polypeptide chains.

It is to be understood non-polar amino acids include amino acids with aliphatic side chains and amino acids with aromatic side chains. The amino acid proline is classified as non-polar but it also has the property of being rigid and can cause changes in secondary structure. For example, prolines are often found at the end of helices. Also, depending on the specific context of the side chain of a given amino acid residue, for example the amino acid tyrosine, generally classed as non-polar due to its aromatic ring, may have analogous functional effects to a polar amino acid residue such as threonine via its hydroxyl group. Thus, tyrosine may be considered to be both a non-polar and a polar amino acid for the purposes of the invention. Furthermore, amino acids which are described as polar or hydrophilic may be uncharged or charged, and may also be basic or acidic. The amino acid histidine is well known to have a pKa value near 7, so that at neutral pH depending upon the protein environment, it may or not be protonated on its side chain, and thus may or not carry a charge. Thus, histidine may be considered to be both a polar charged or a polar uncharged amino acid residue for the purposes of the invention.

The mutations discussed herein are generally introduced into the protein by using methods known in the art, such as site directed mutagenesis of the protein, PCR and gene shuffling methods or by the use of multiple mutagenic oligonucleotides in cycles of site-directed mutagenesis. Thus, the mutations may be introduced in a directed or random manner. The mutagenesis method thus produces one or more polynucleotides encoding one or more different mutants.

The development of a recombinant baculovirus containing the Porcine Circovirus 3 ORF2 gene under control of the baculovirus polyhedrin promoter (BaculoG/PCV3 ORF2 Clone 4B4-2E12 Pre-MSV p8; lot no. 3624-039) is described in Example 1. In some embodiments, the use of such a recombinant baculovirus described in Example 1 in a vaccine may encompass killed and/or inactivated versions of the recombinant virus. Alternatively, in some vaccines, a recombinant virus, for example similar to that shown in Example 1, may be used as a live, modified virus.

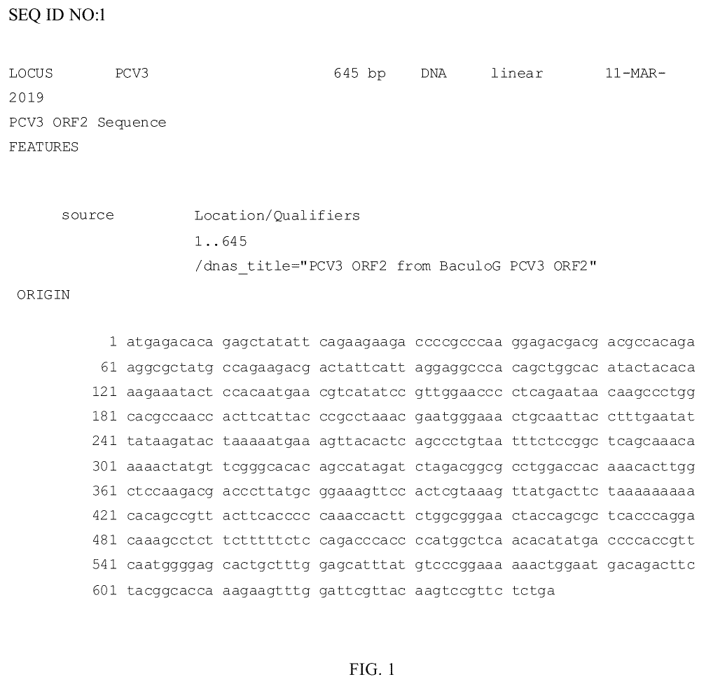

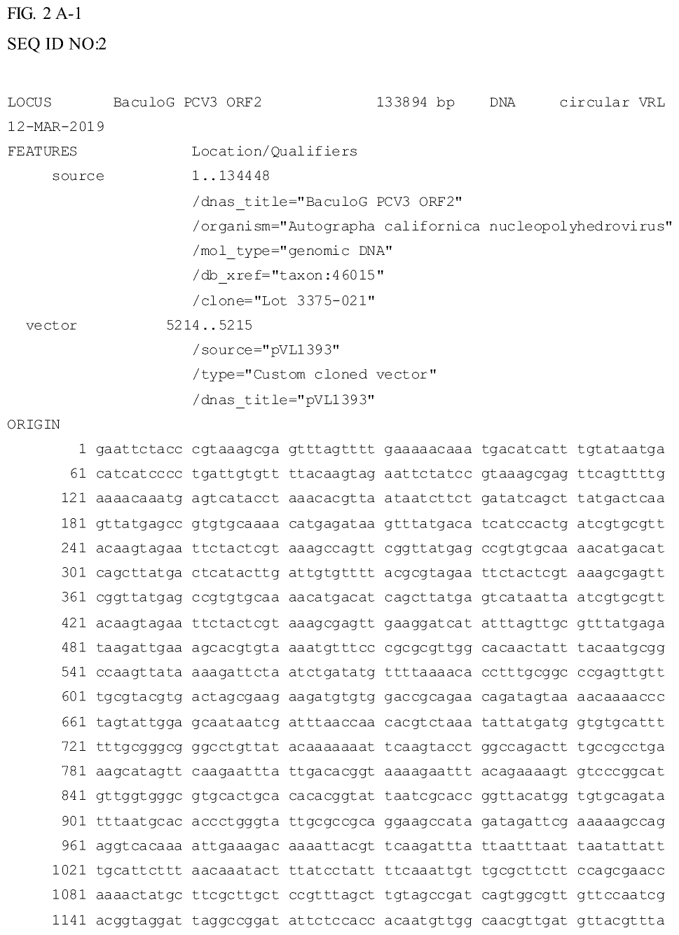

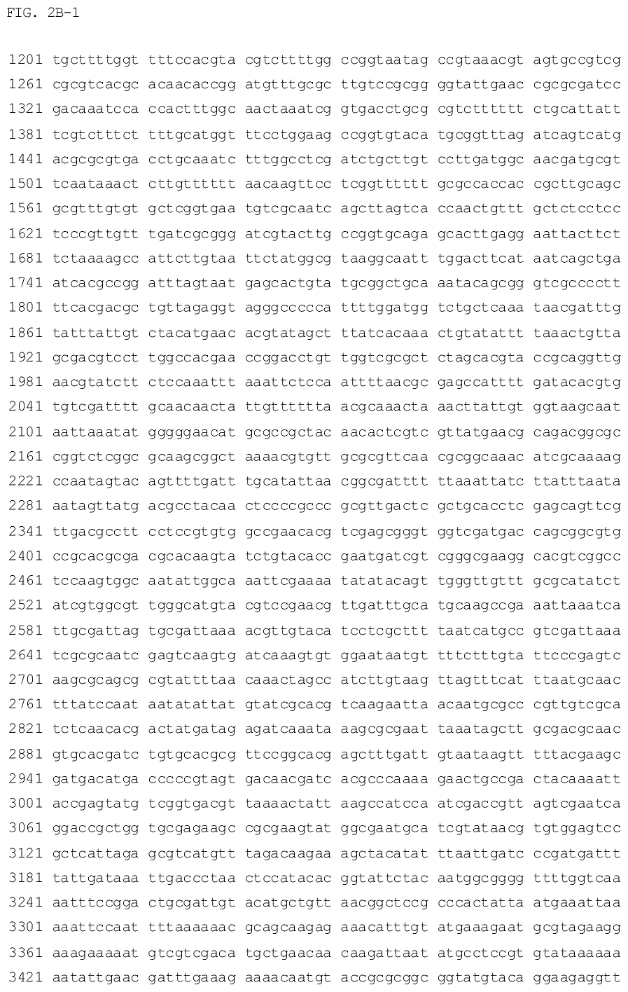

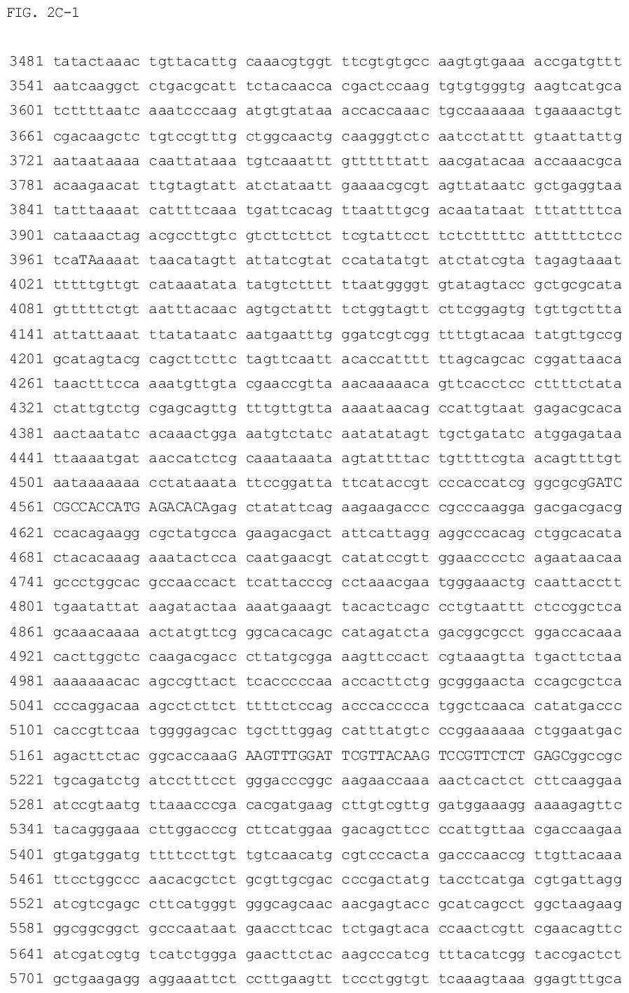

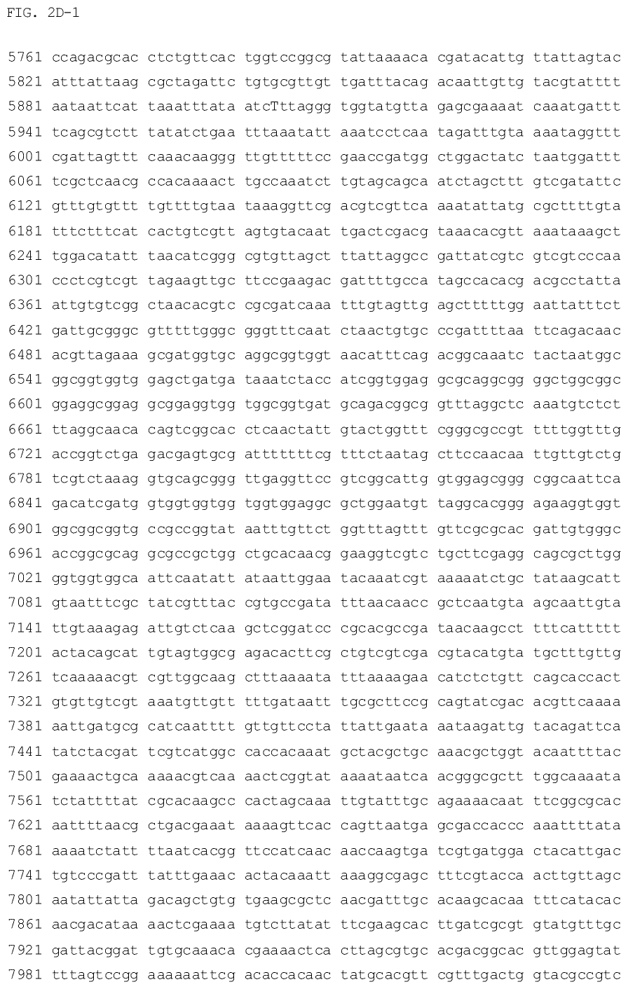

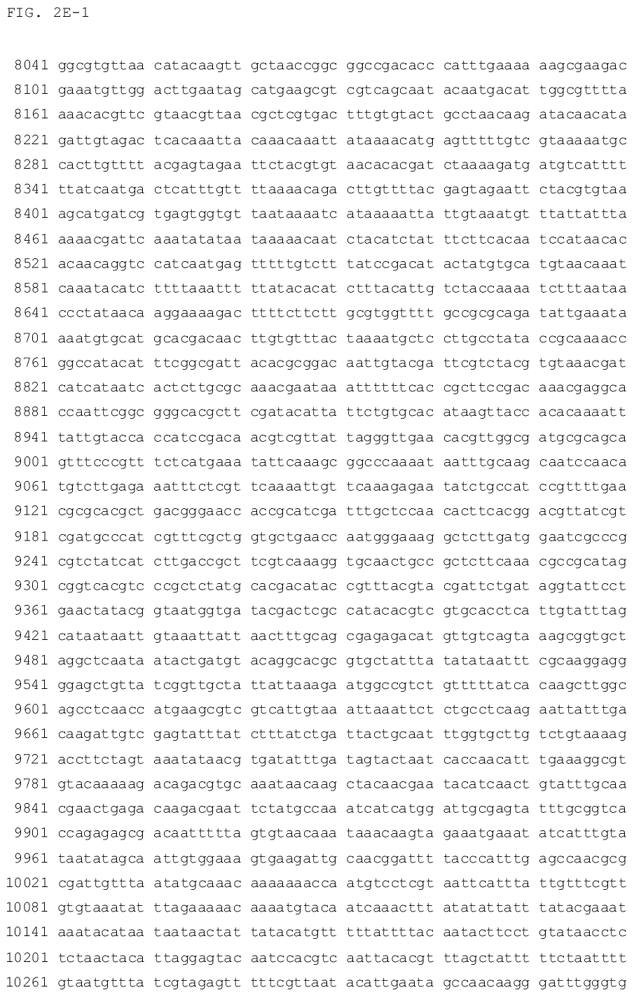

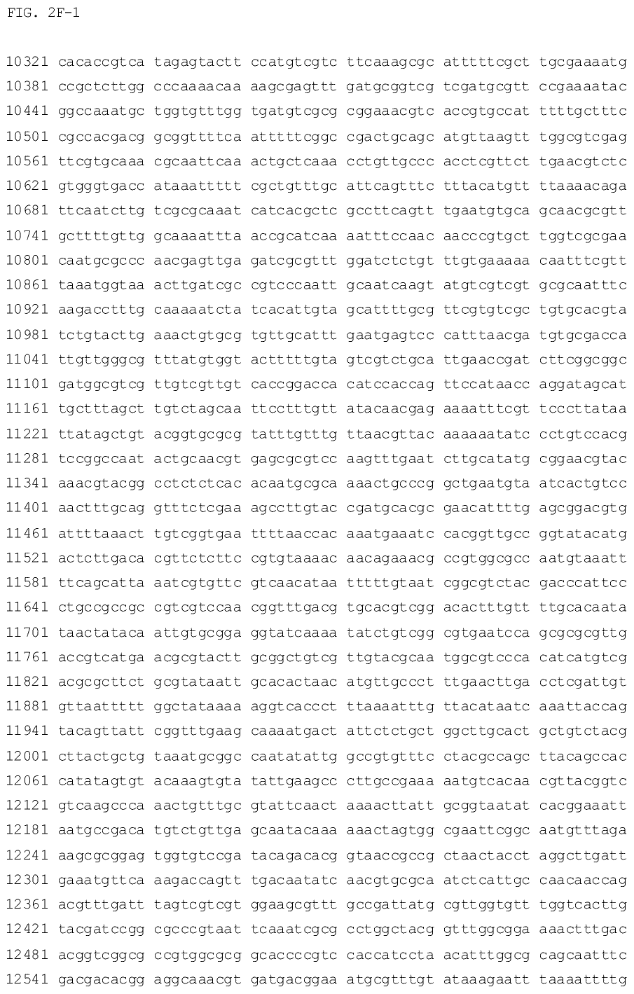

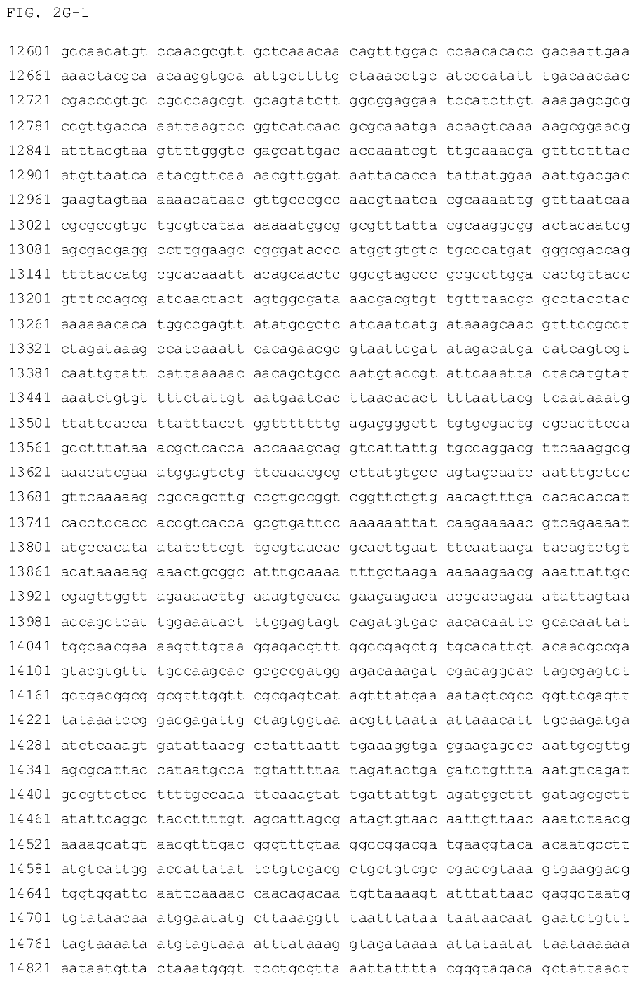

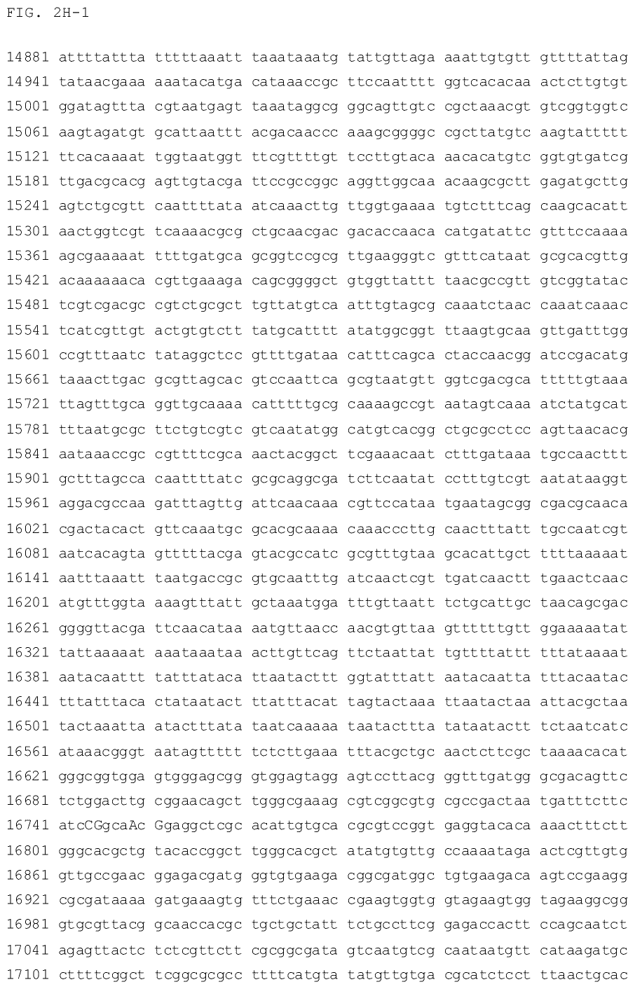

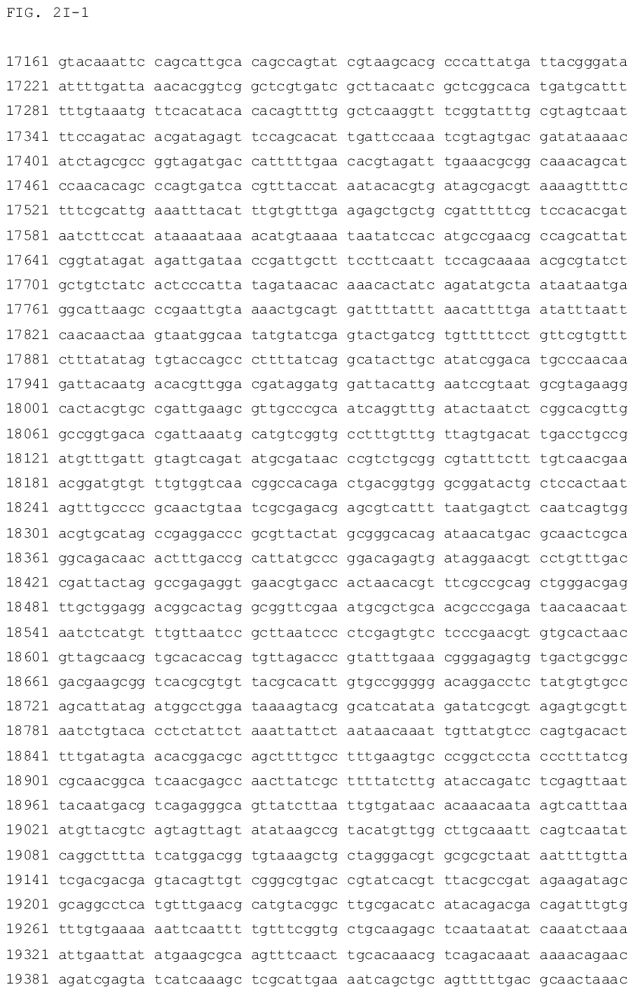

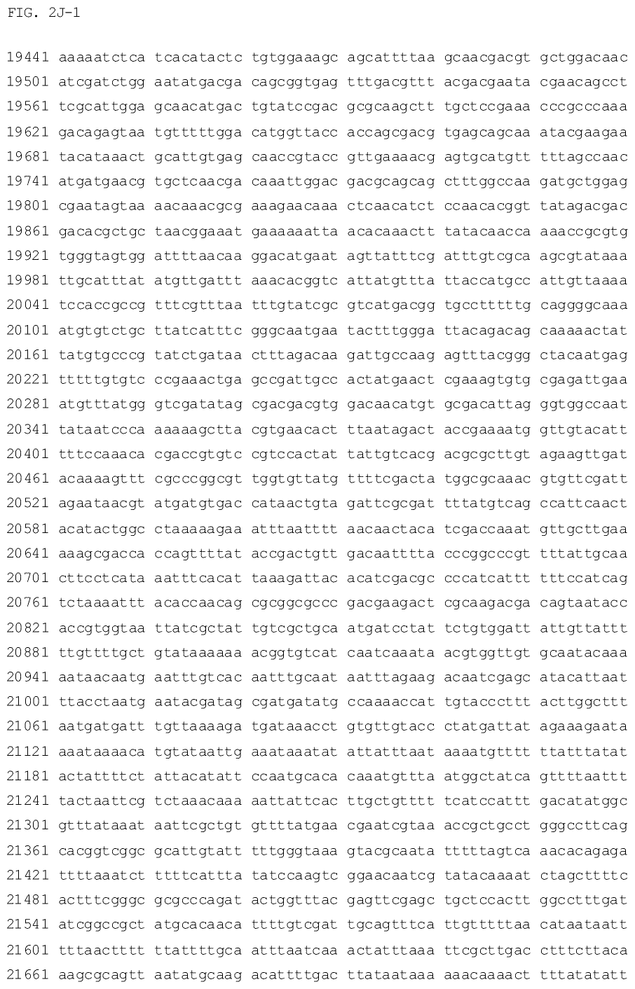

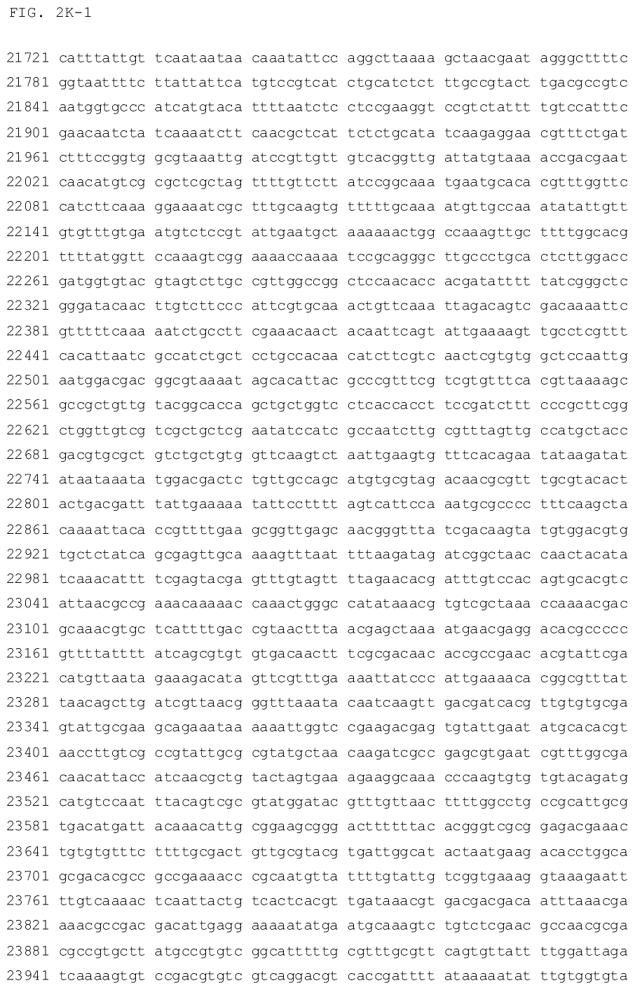

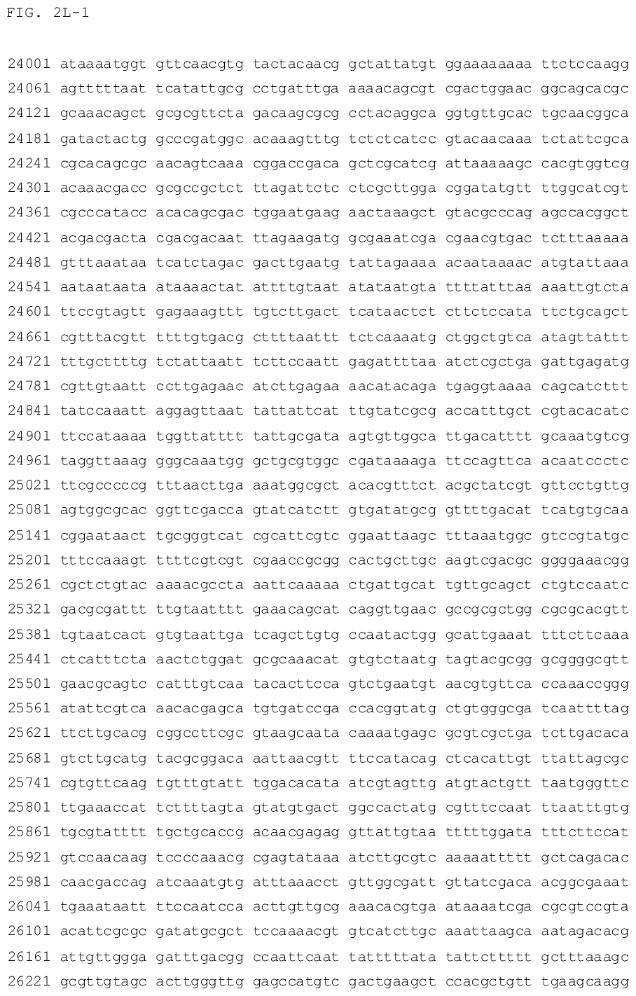

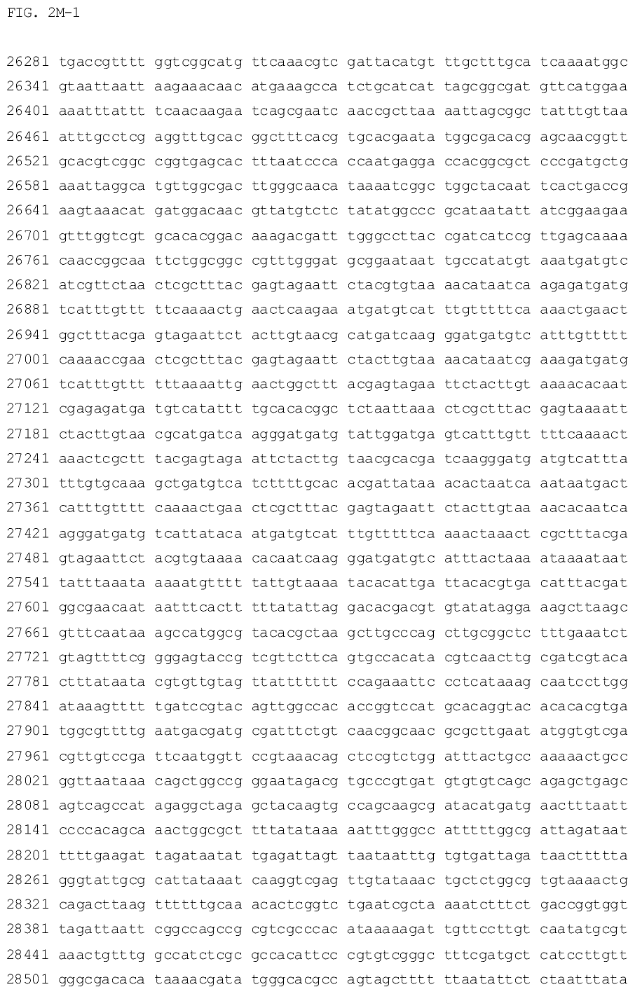

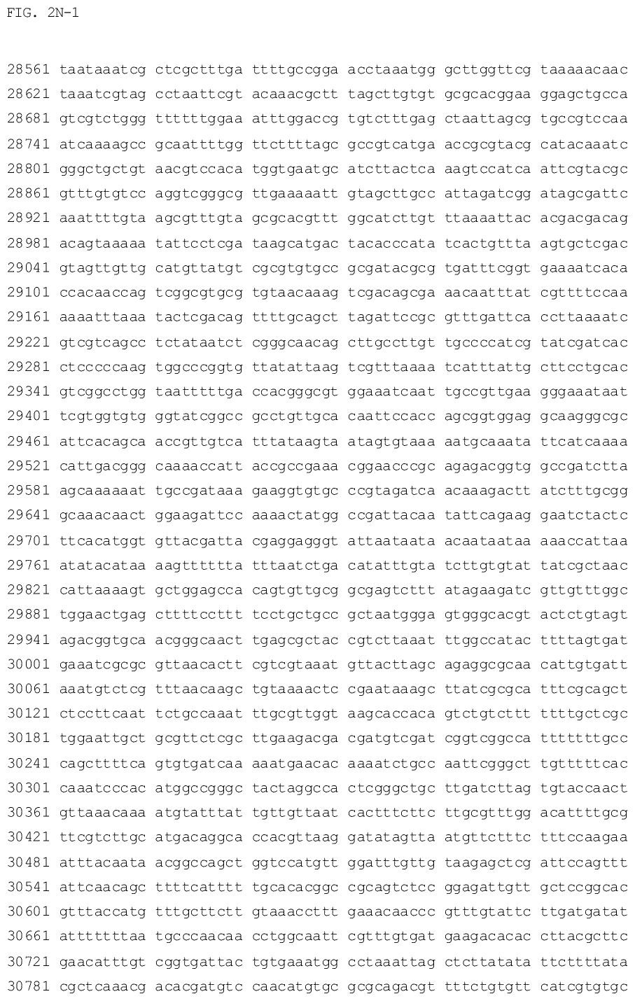

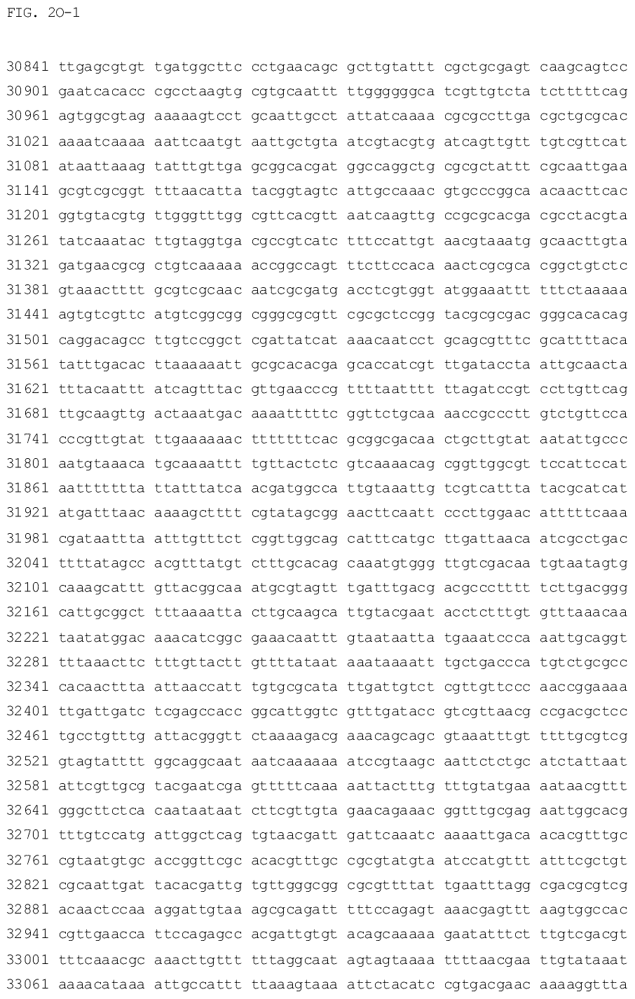

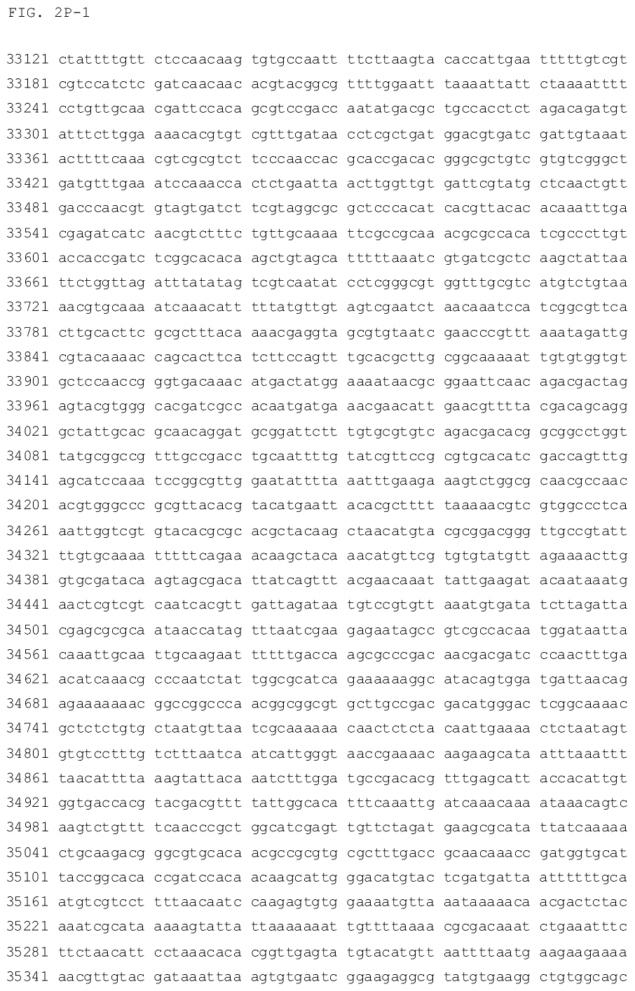

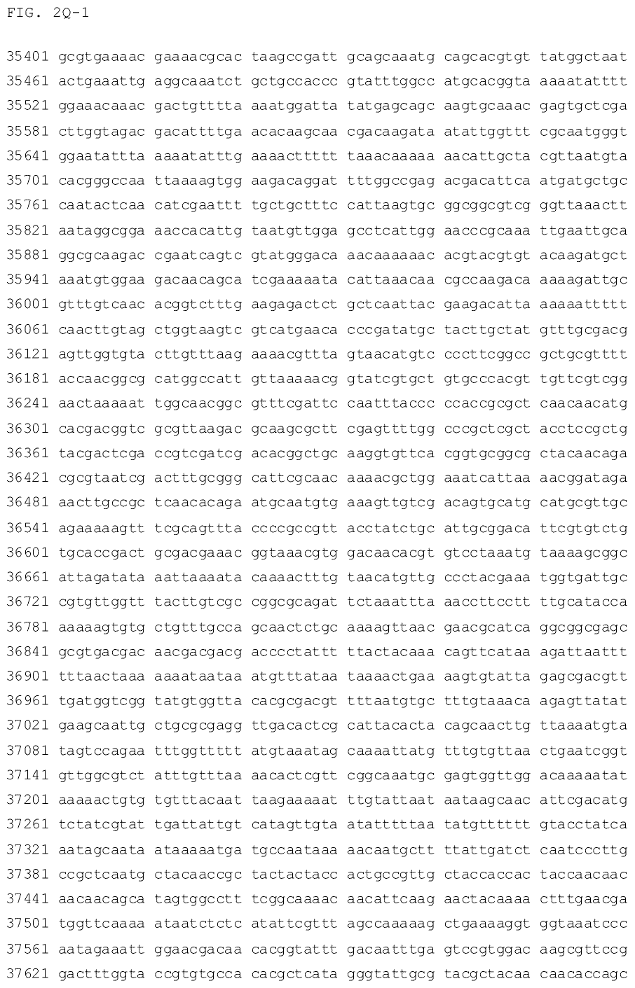

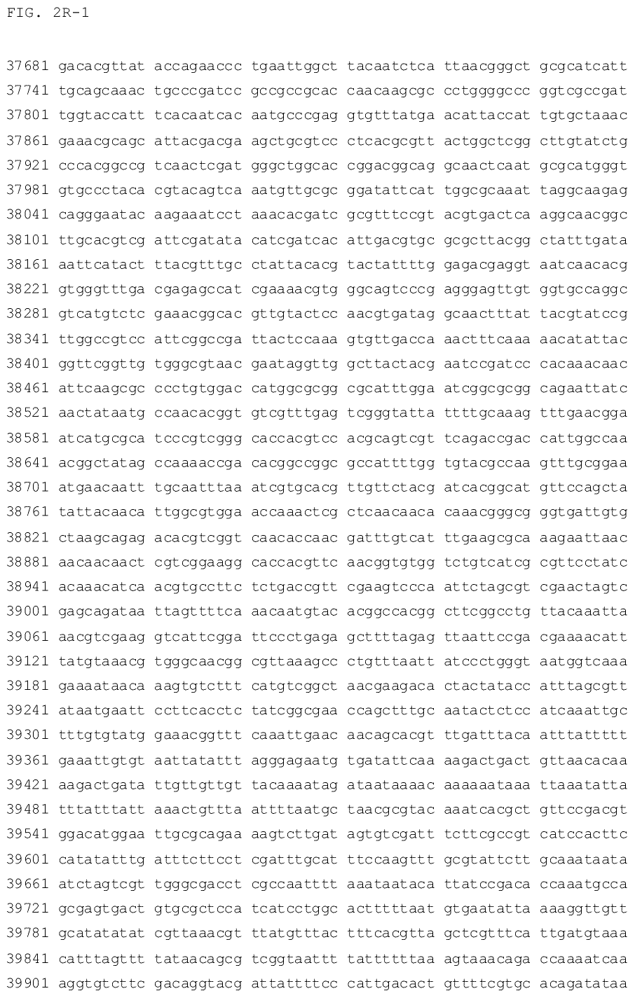

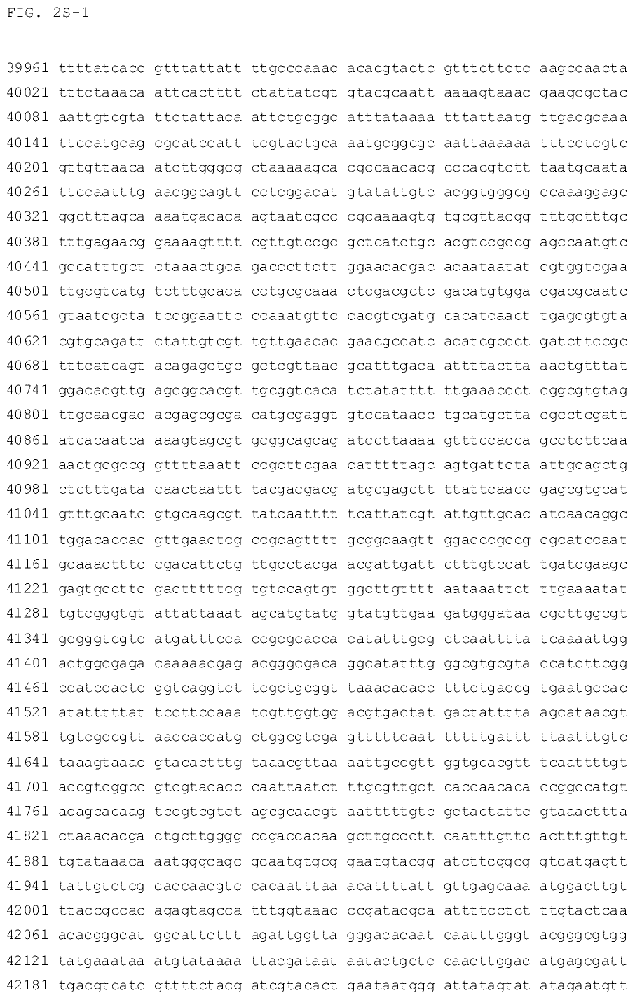

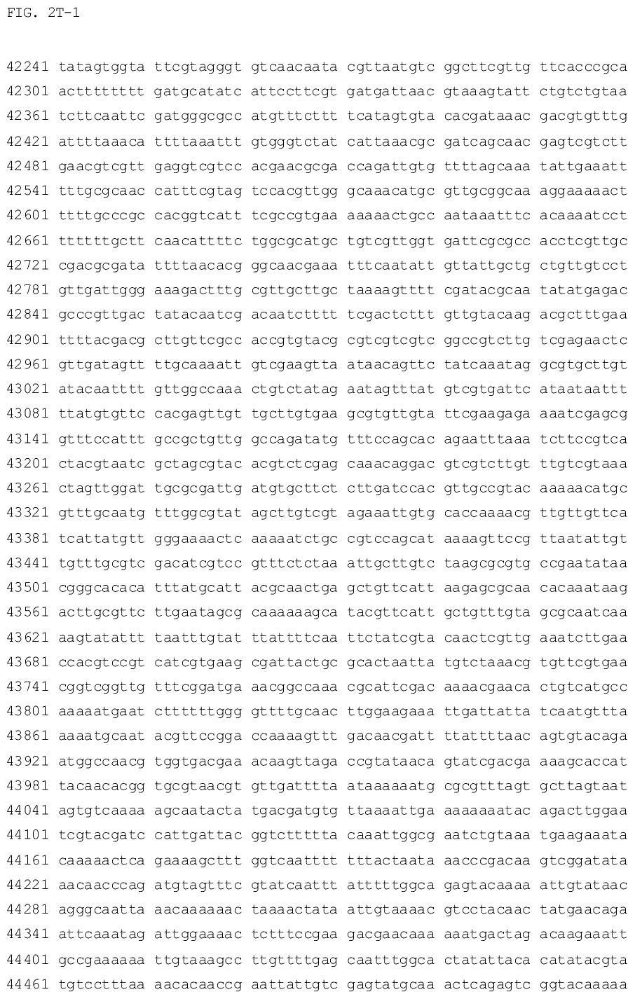

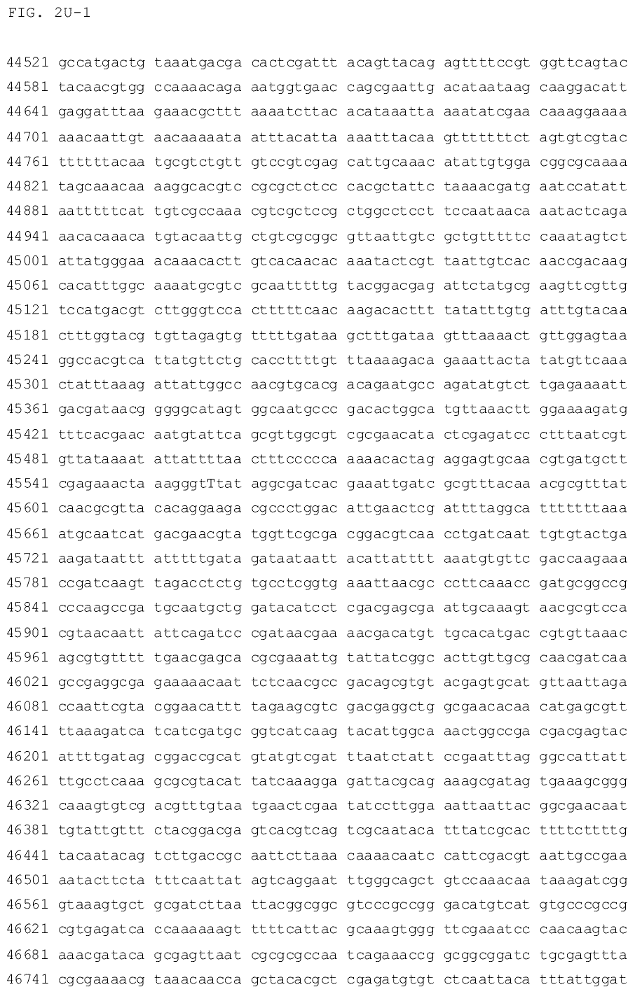

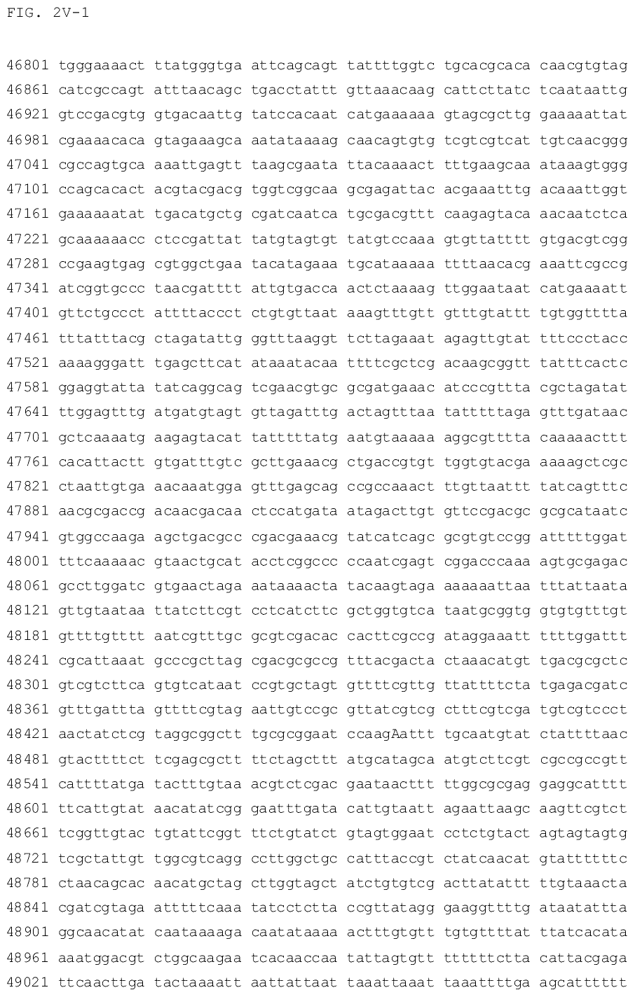

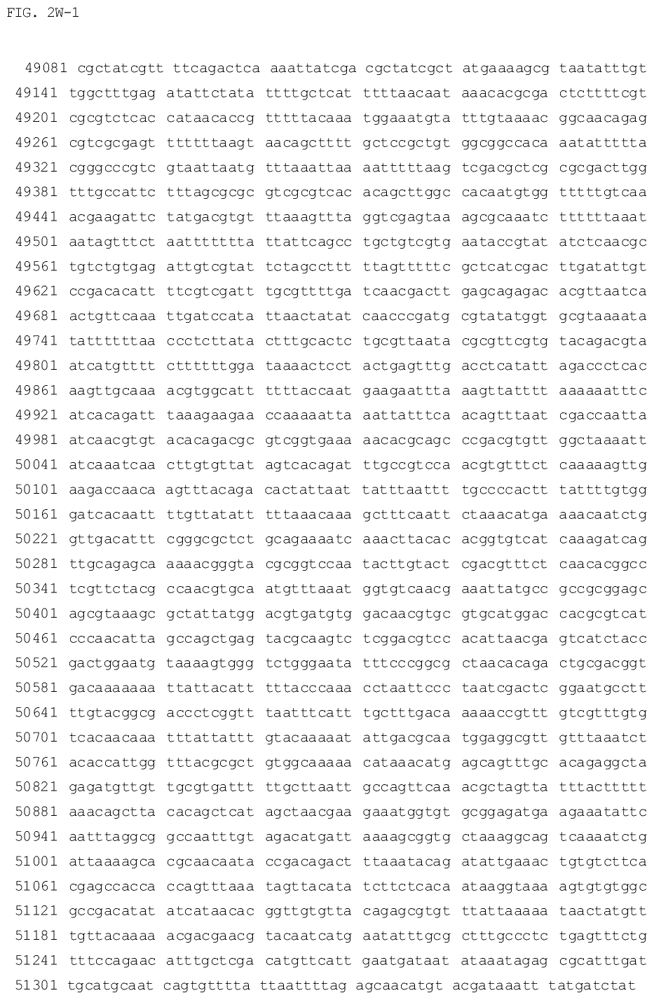

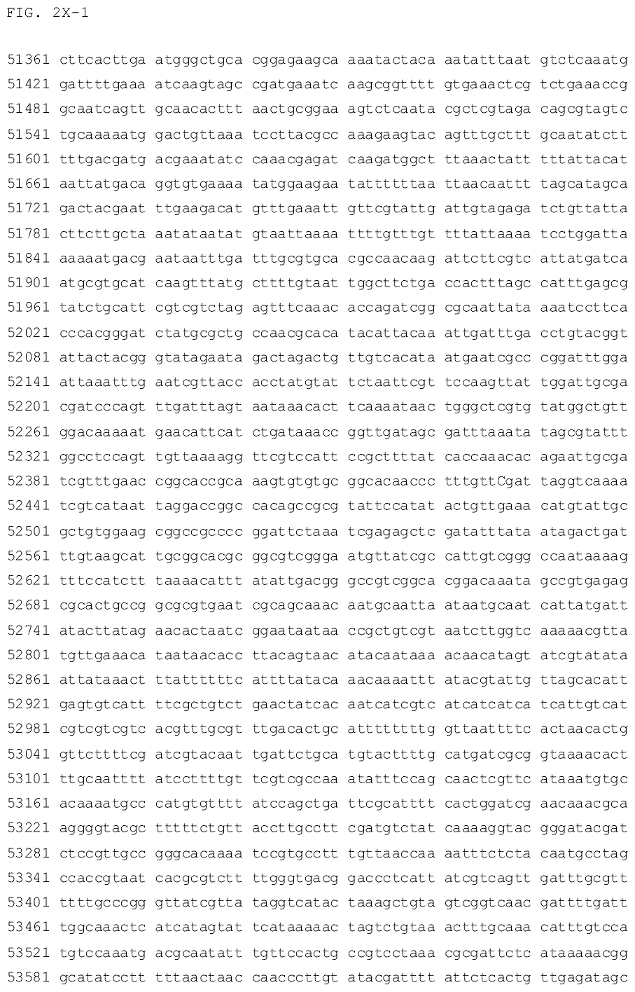

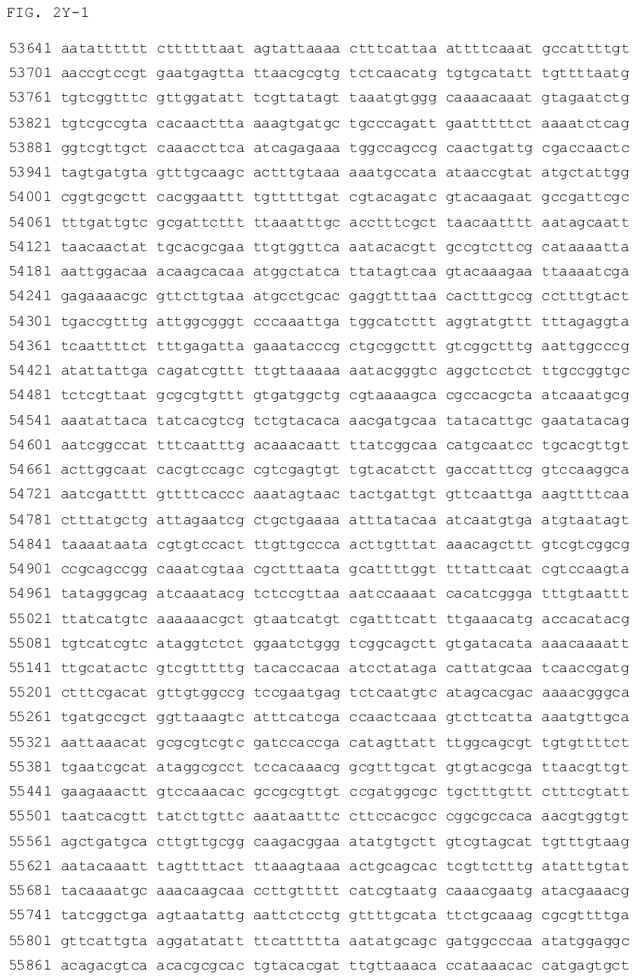

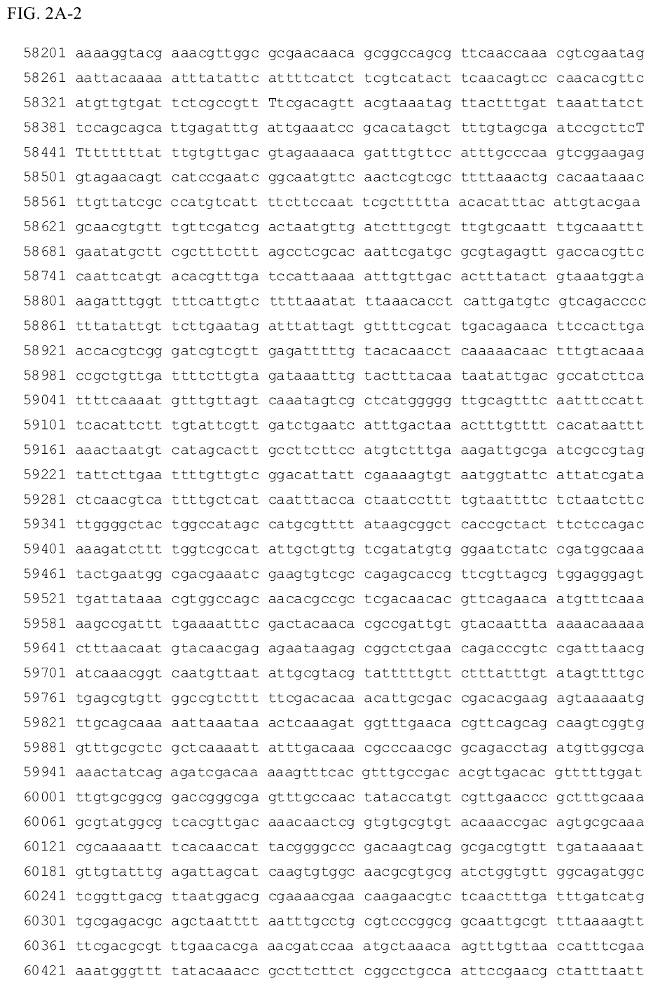

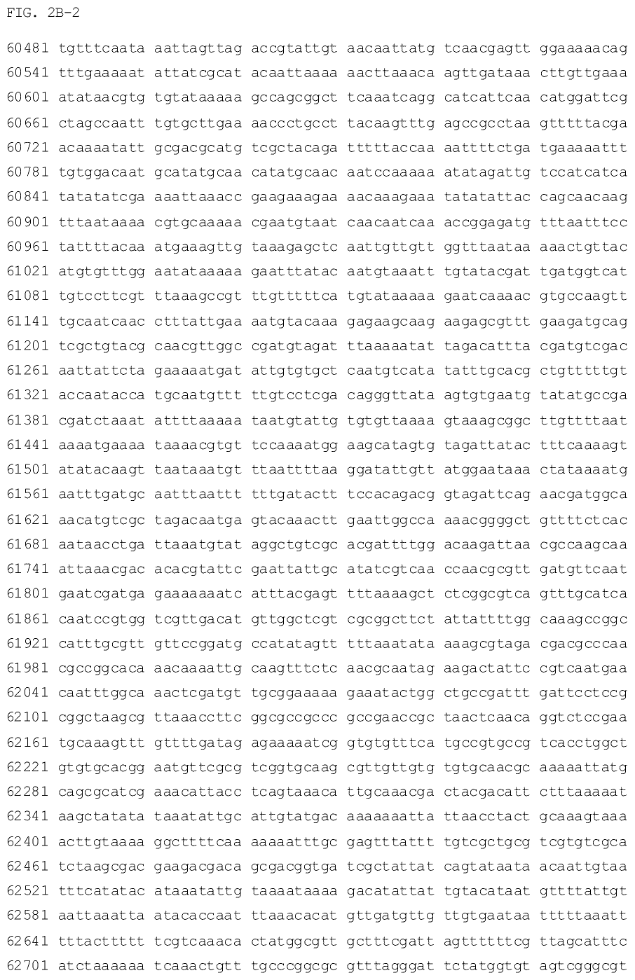

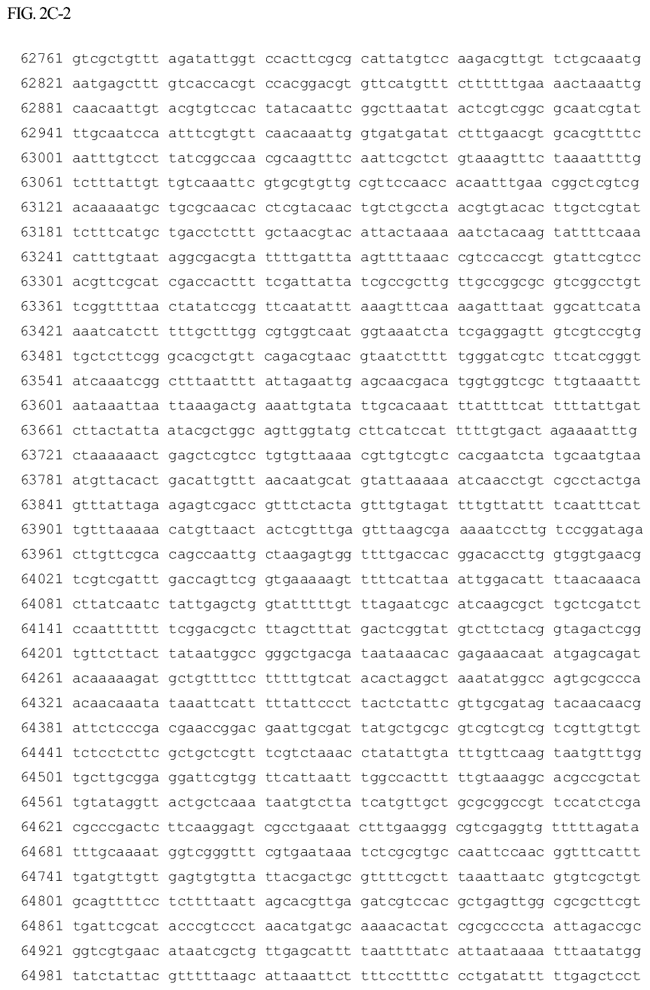

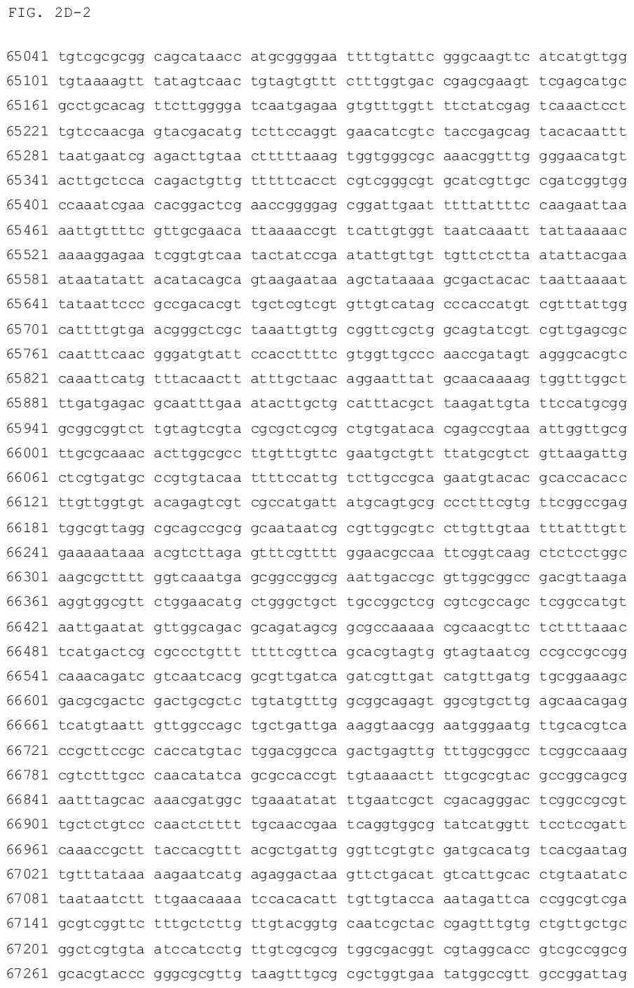

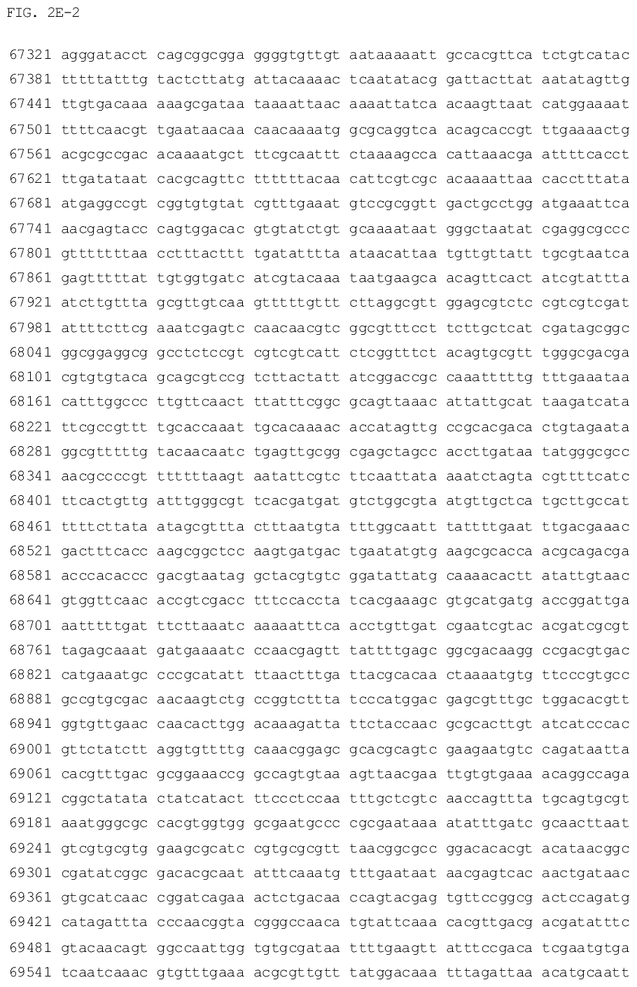

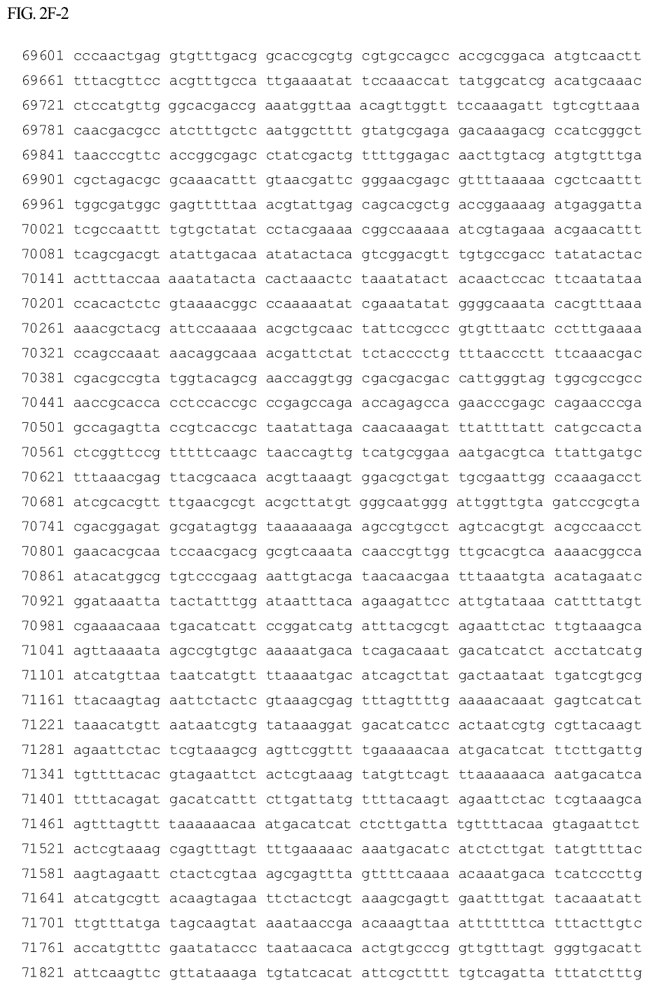

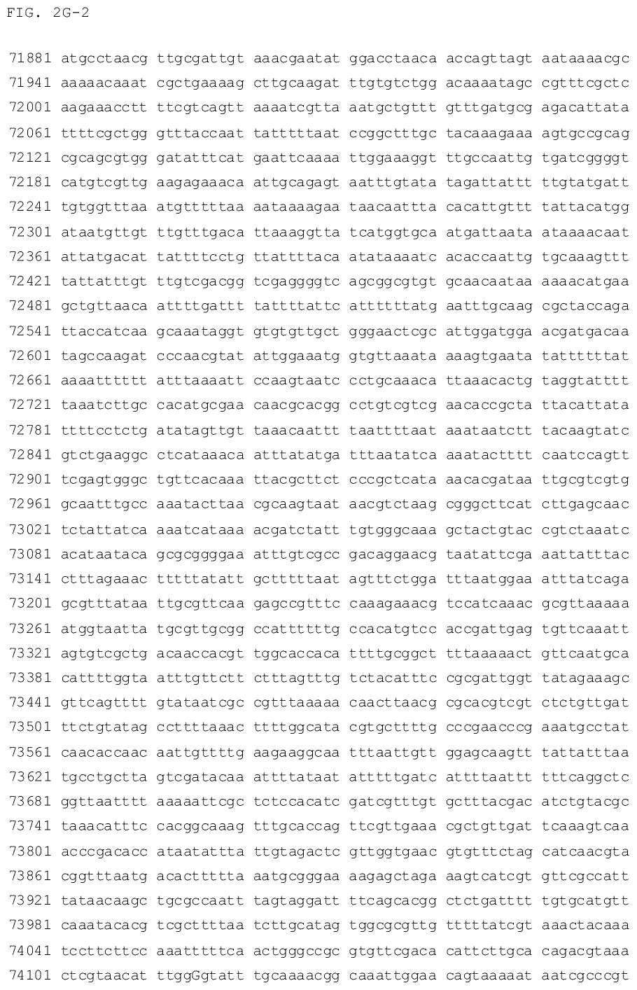

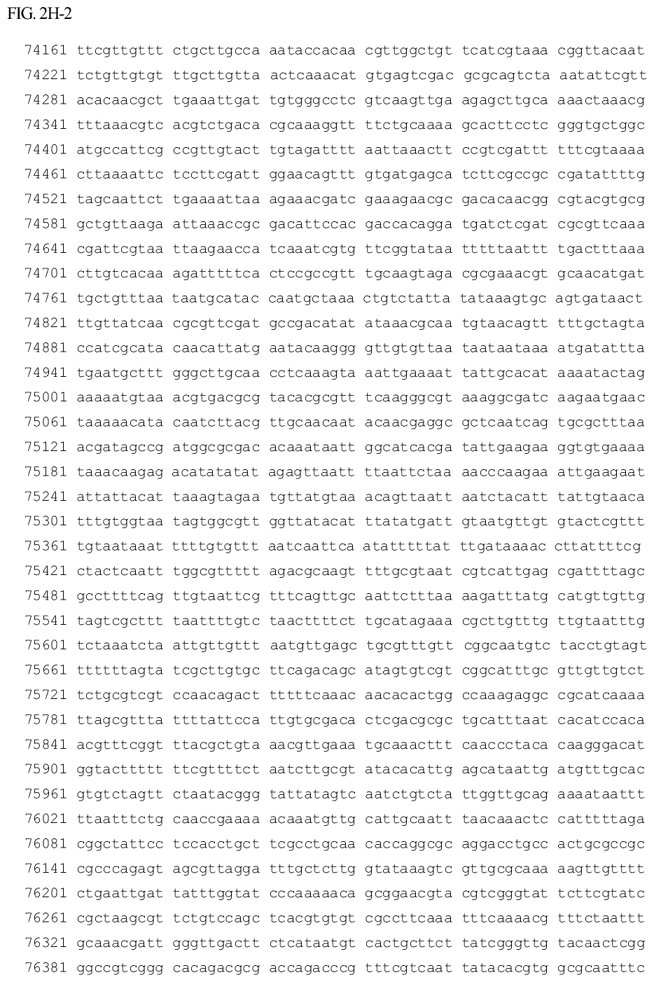

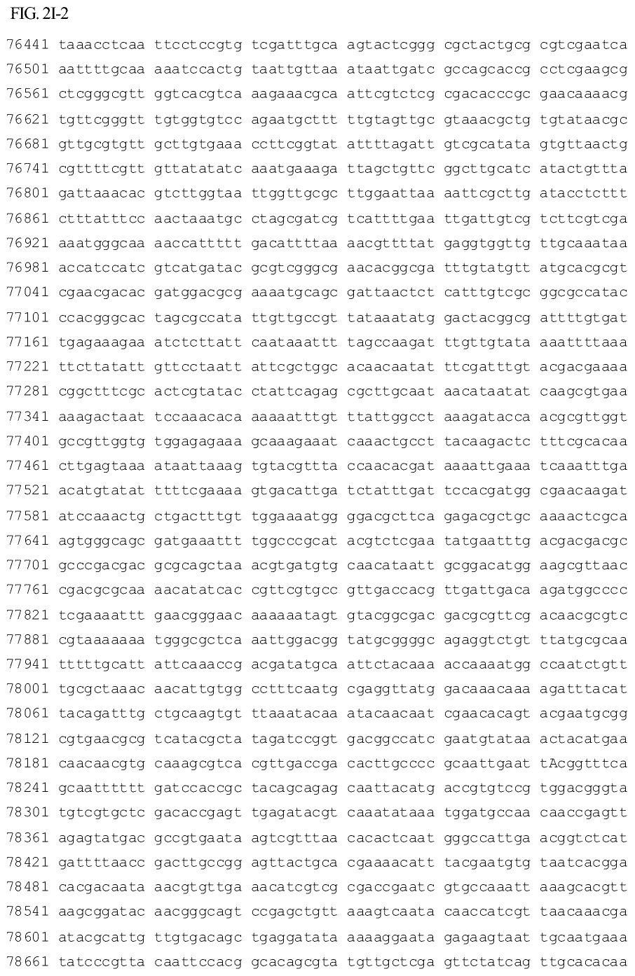

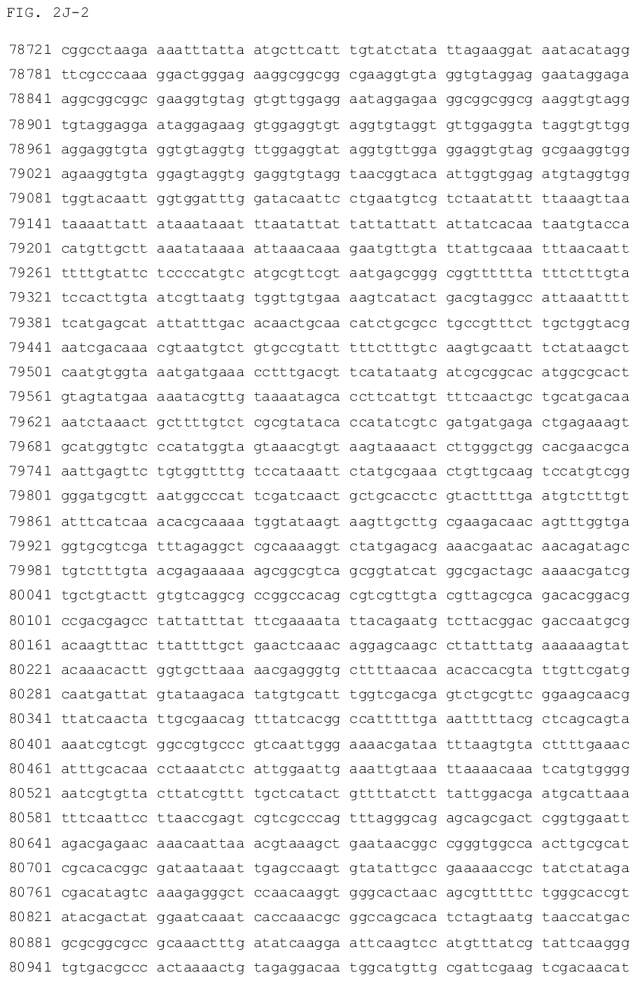

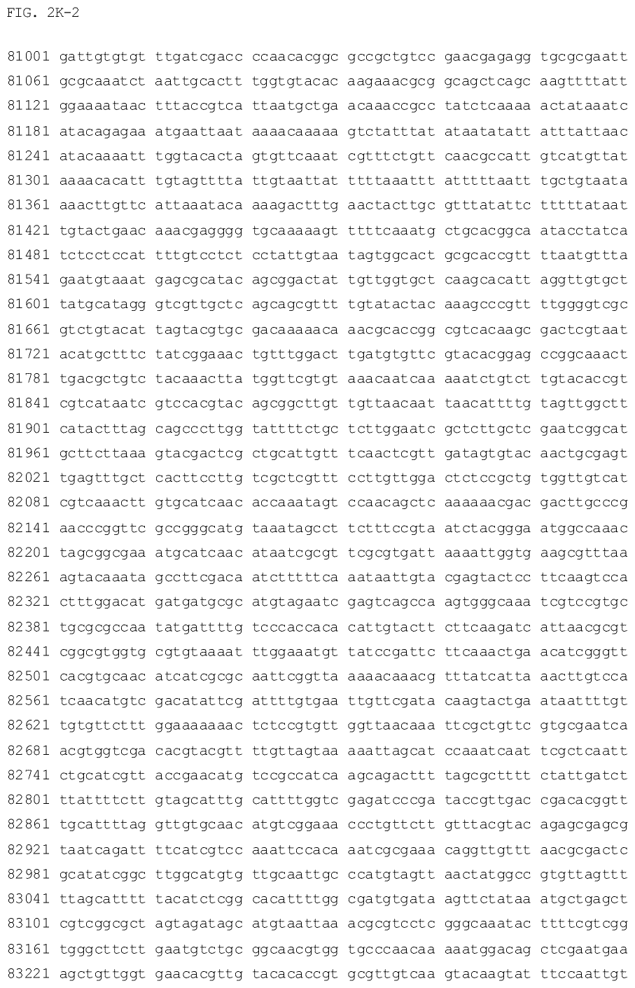

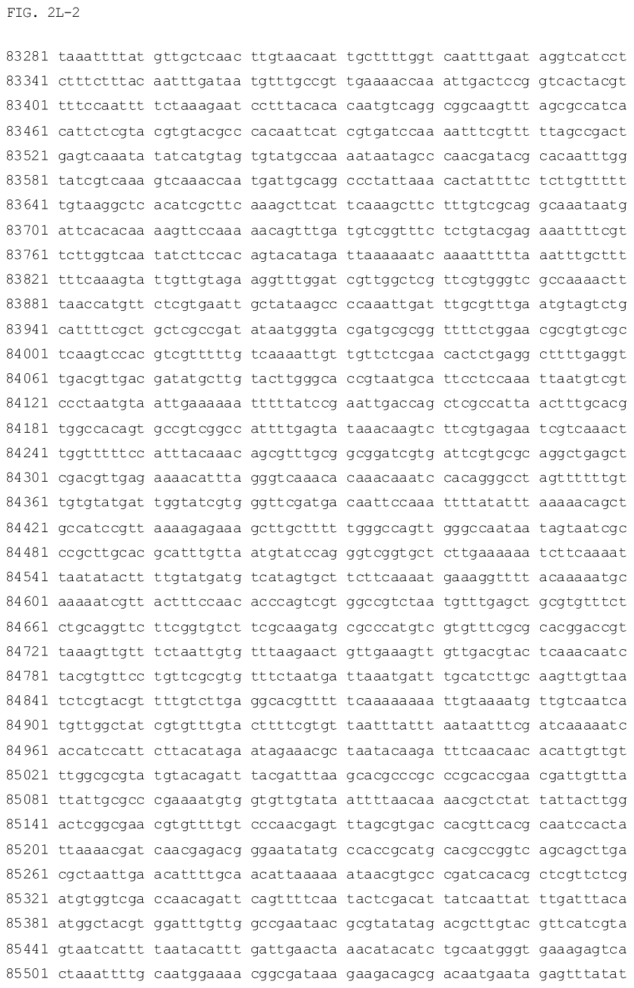

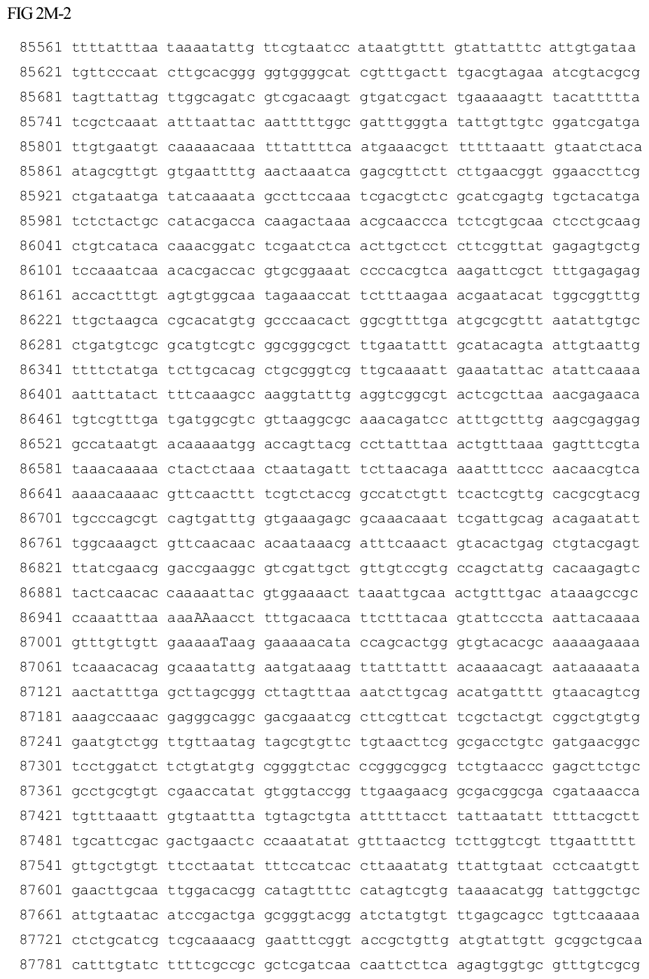

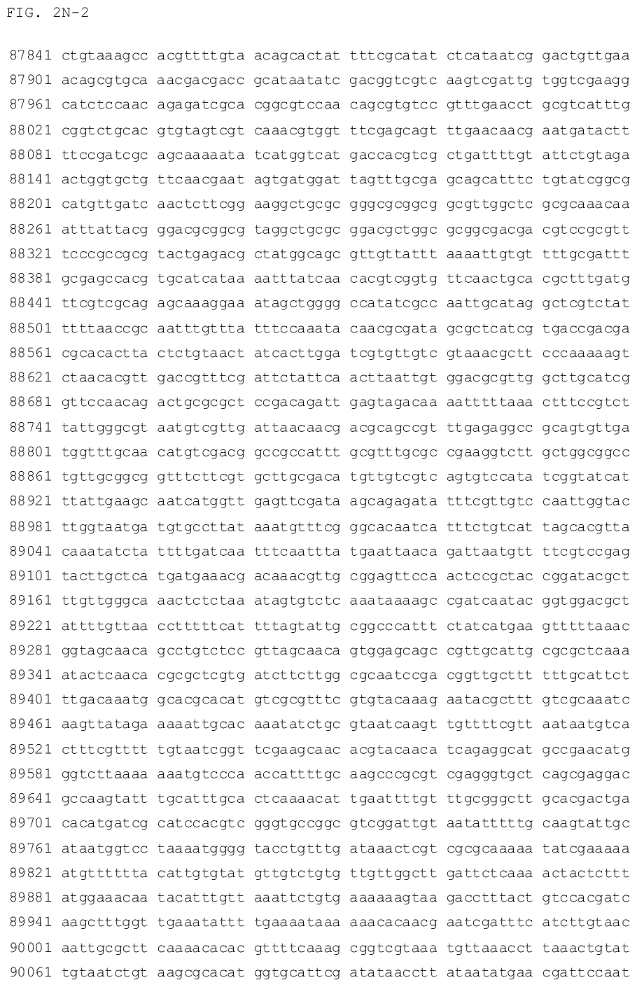

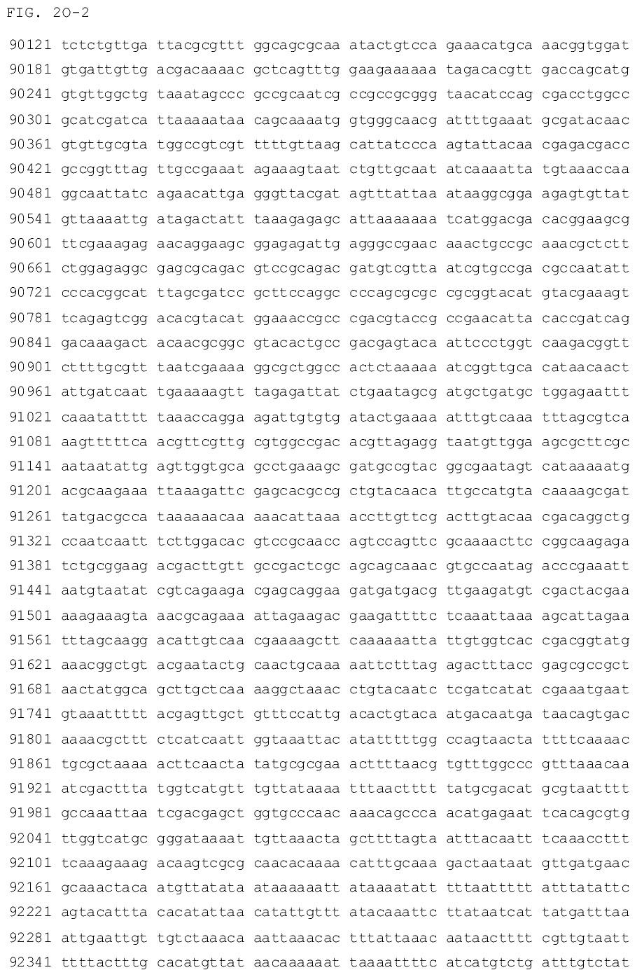

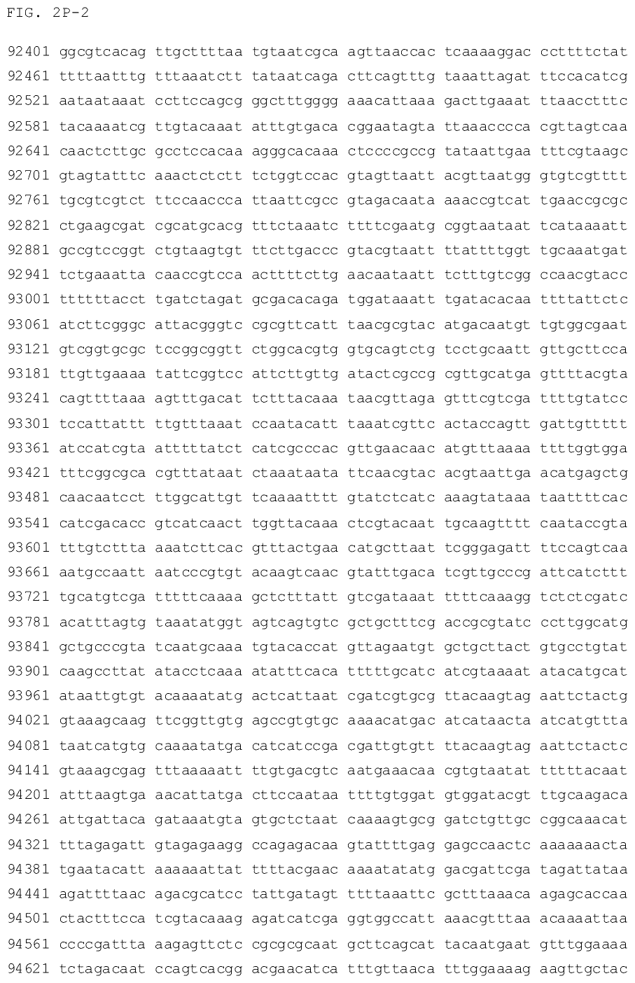

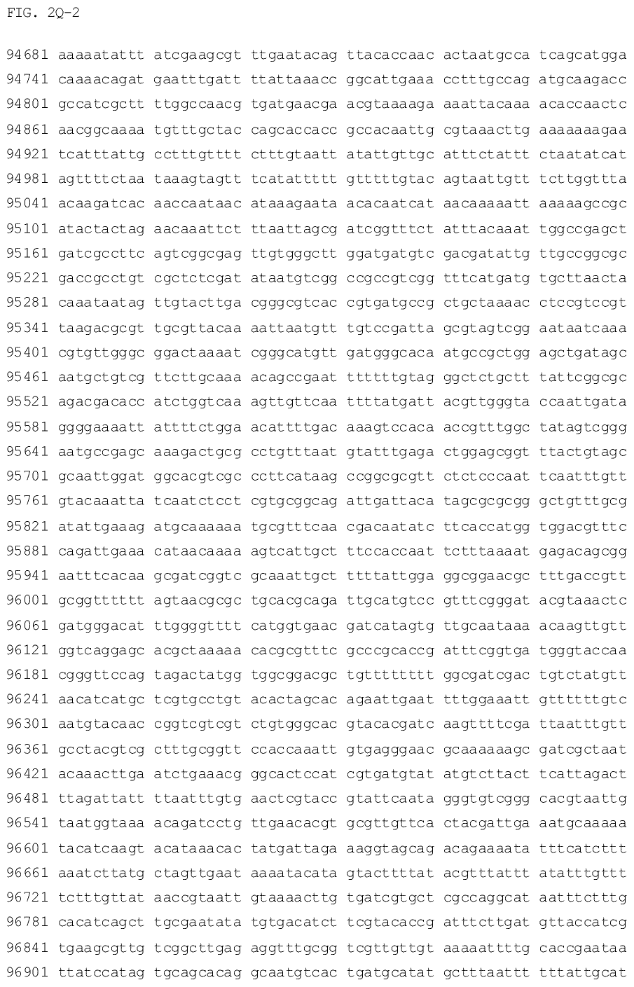

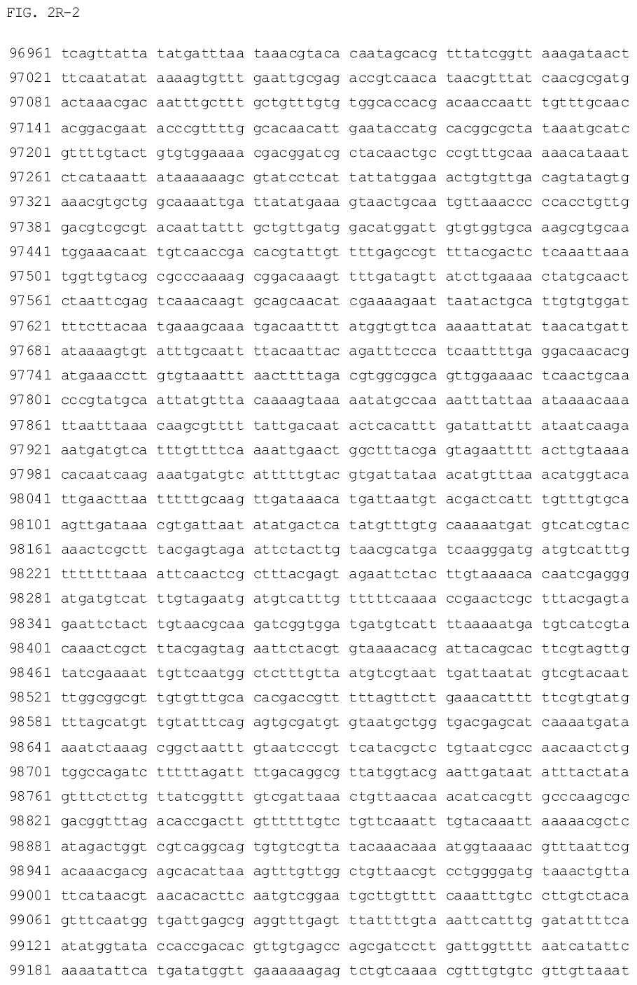

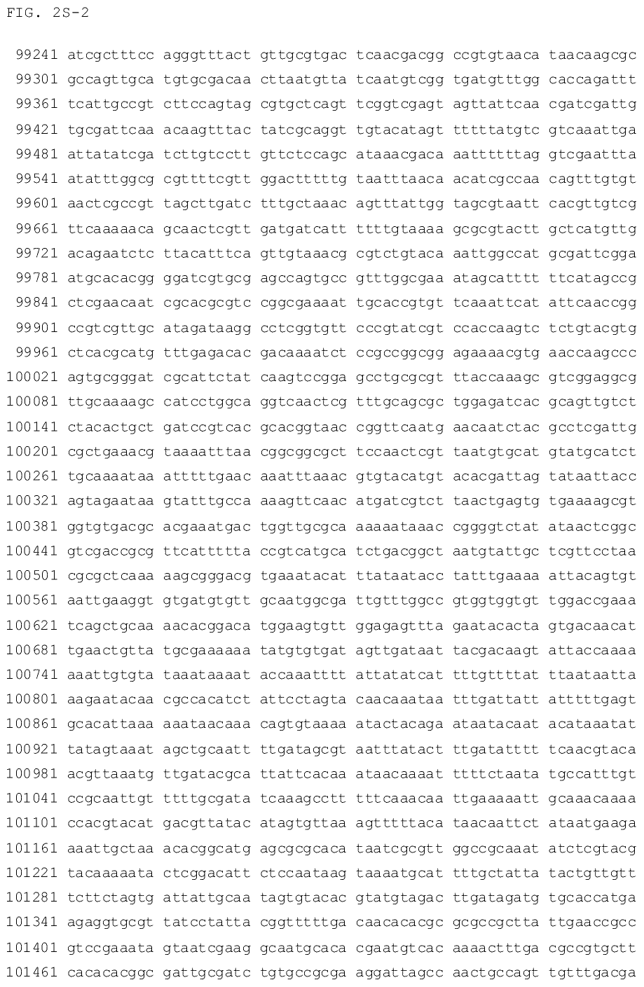

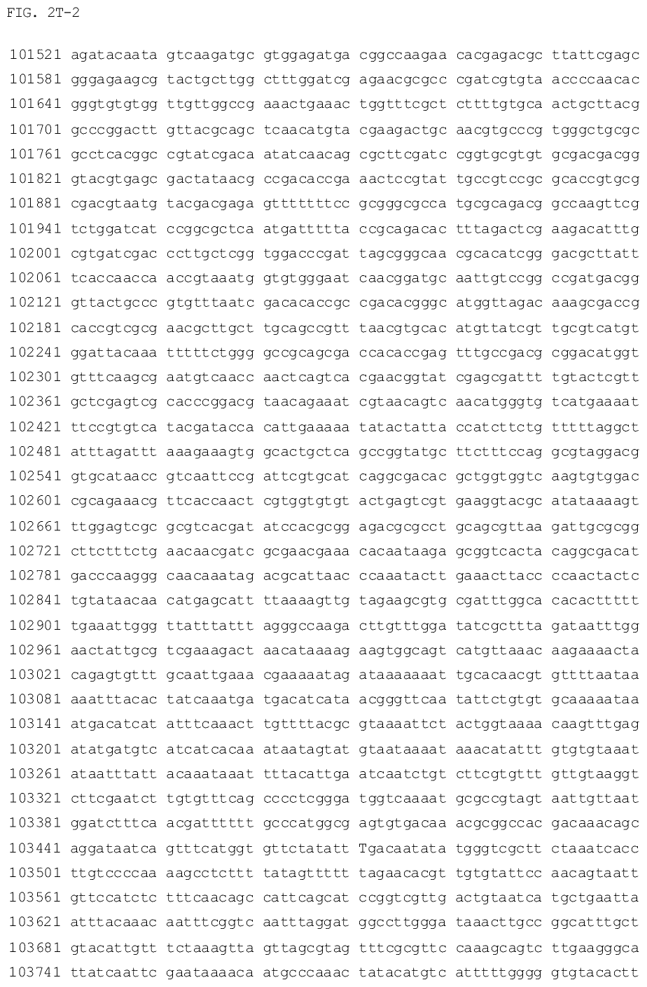

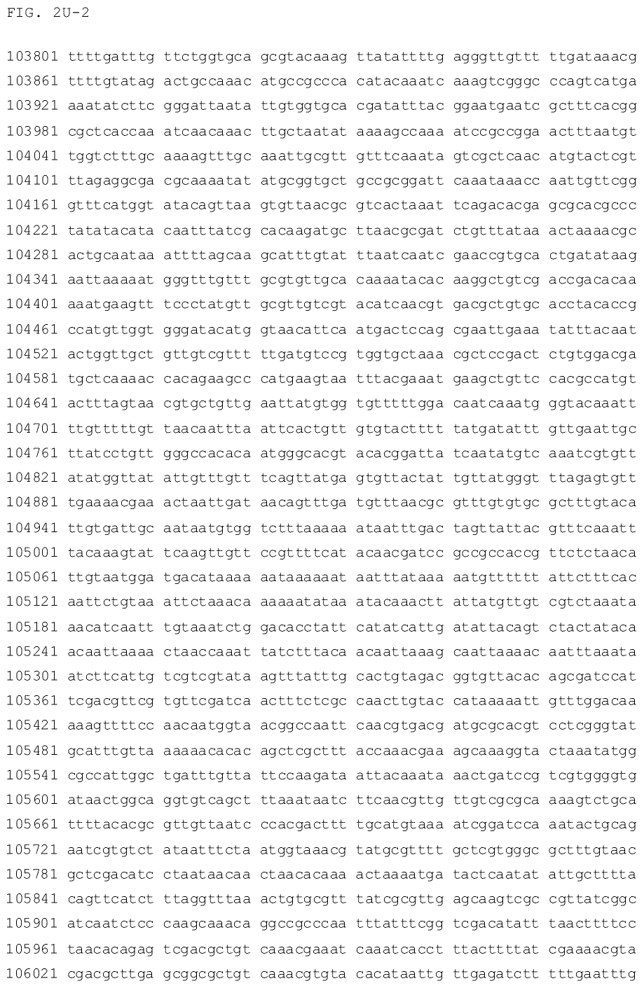

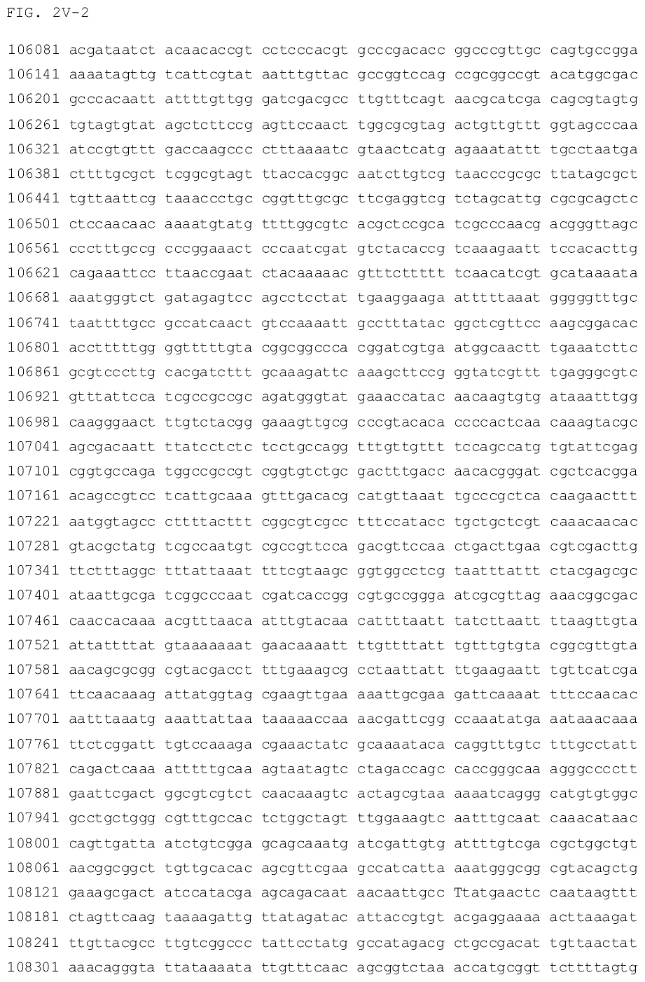

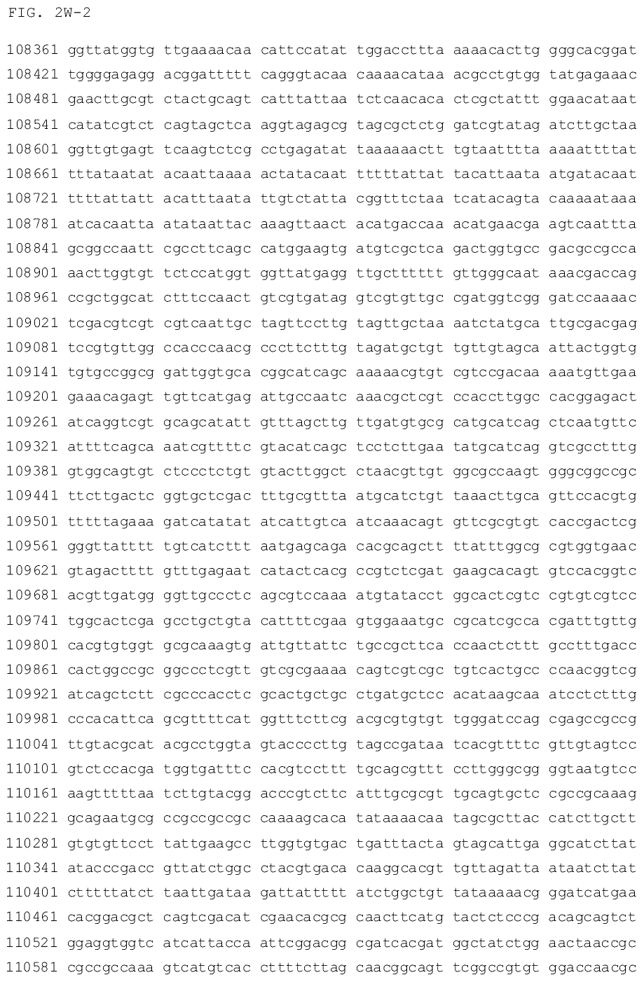

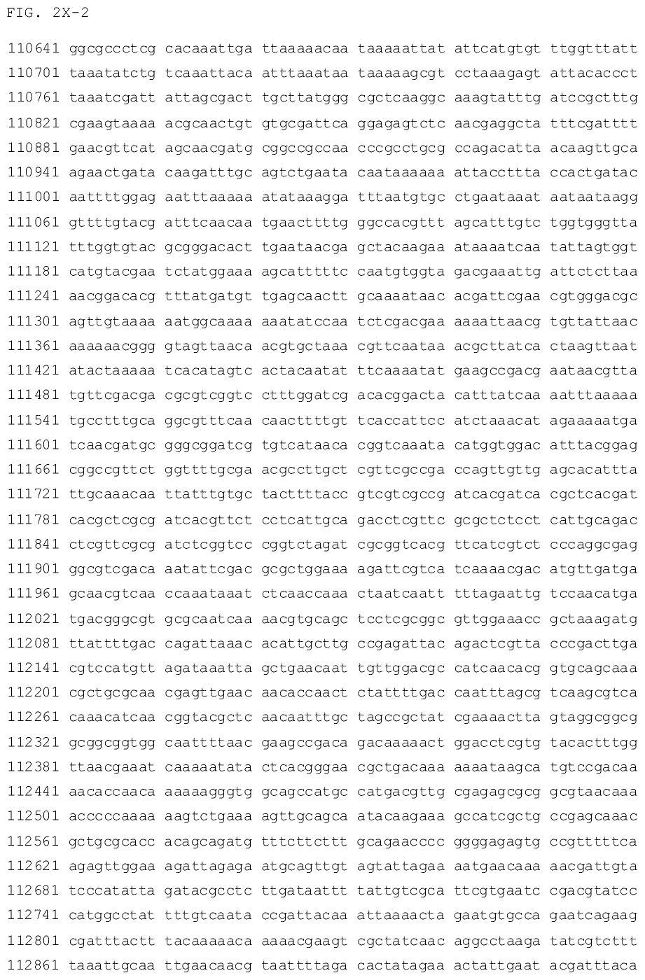

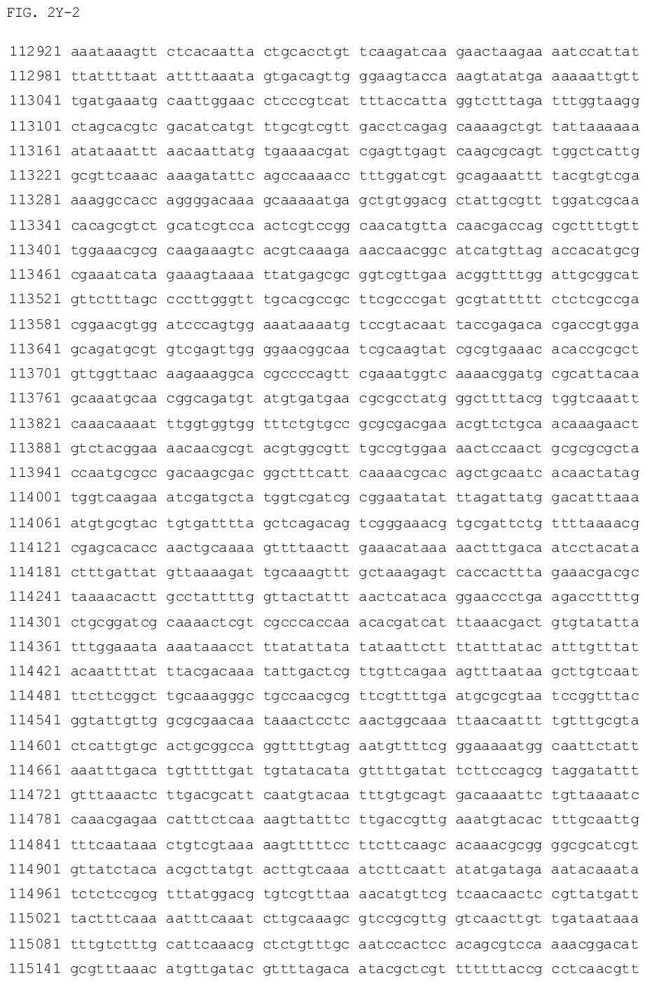

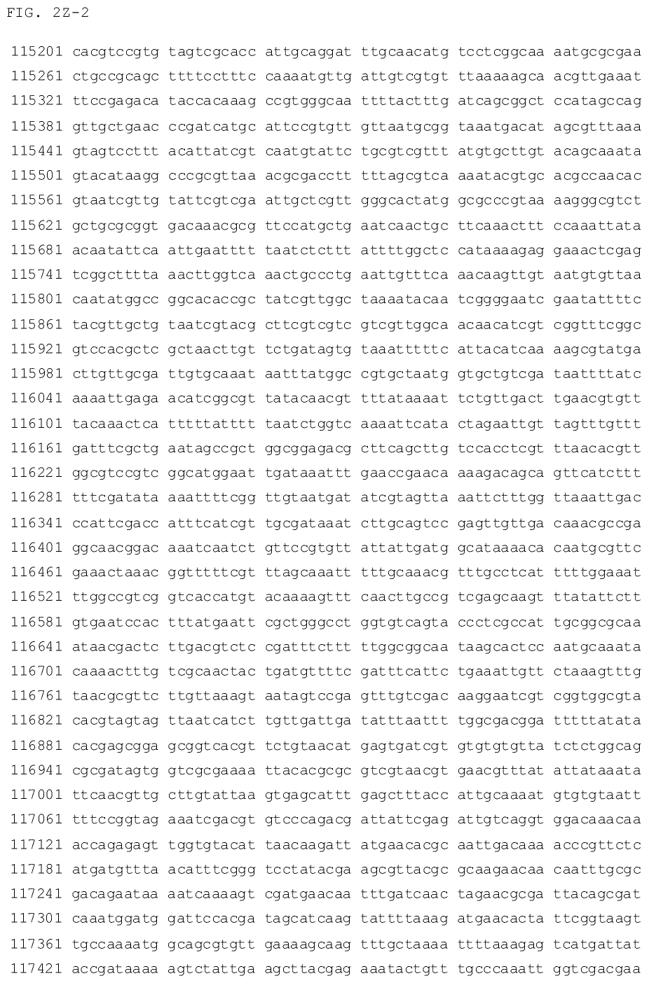

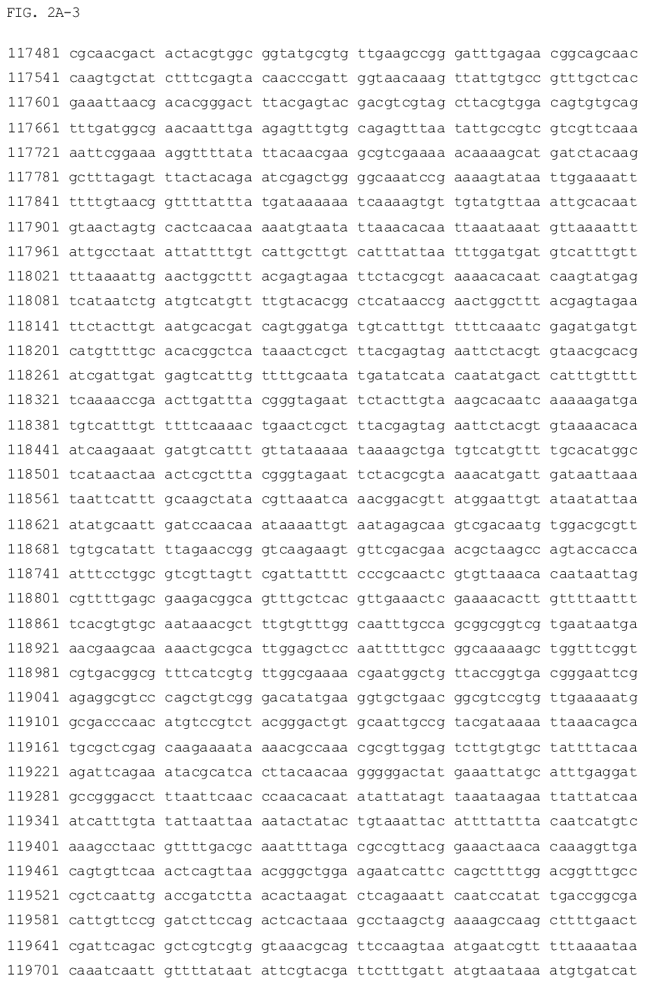

FIG. 1B provides the sequence of the recombinant baculovirus BaculoG/PCV3 ORF2, SEQ ID NO:2. The backbone sequence annotations are from Genbank accession NC_001623. One of skill in the art will appreciate that minor mutations in the backbone from construct to construct is to be expected given the complexity of the DNA sequence. A map of the construct is shown in FIG. 2. The baculovirus expression vector, BaculoG/PCV3 ORF2, may be used to develop PCV3 vaccines and/or controls. Preferred adjuvants for a given vaccine and/or control may differ based on the type of expression vector used, for example, live, live modified, inactivated, or killed. Adjuvant effectiveness may vary based on the status of the vector (e.g., virus) used. An amount of adjuvant used in a vaccine may be predetermined, for example, a predetermined percentage may be selected to be within a given range (e.g., weight percentage and/or volume percentage in the vaccine) for a given adjuvant and/or combination of adjuvants. In some instances, for example, when using live vaccines multiple adjuvants may be used. For example, in some embodiments, a combination of adjuvants such as Carbopol® and Montanide ISA 207VG may be used. Alternatively, a vaccine that includes a live expression vector, such as BaculoG/PCV3 ORF2, may be adjuvanted with ISA 207VG and/or Carbopol®. For example, the adjuvant may be present in the vaccine at a predetermined concentration. For example, a vaccine may include a concentration of 50% ISA 207VG by weight of the vaccine. Alternatively, another vaccine including live BaculoG/PCV3 ORF2 may include an adjuvant, such as Carbopol® at 20% by volume of the vaccine.

Vaccines that include killed expression vectors, such as viruses, may include carbopol as an adjuvant. For example, a vaccine that includes killed BaculoG/PCV3 ORF2 may in some embodiments include Carbopol® as the effective adjuvant. For example, such a vaccine may include a predetermined amount of adjuvant, for example a predetermined weight or volume percentage of the vaccine. In particular, a vaccine that includes killed BaculoG/PCV3 ORF2 may include Carbopol® at 20% by volume of the vaccine. Alternately, a vaccine may include killed BaculoG/PCV3 ORF2 and adjuvant at about 50% of the weight of the vaccine solution. For example, a vaccine that includes killed BaculoG/PCV3 ORF2 may include ISA 207VG as an adjuvant at a predetermined weight percentage of the vaccine, such as fifty percent.

For example, the Baculovirus expression vector BaculoG/PCV3 ORF2, was used to develop two PCV3 vaccines and a control as outlined herein:

Development of BaculoG/PCV3 ORF2, P9; live, adjuvanted with 50% ISA 207VG vaccine (methods used to develop the vaccine are disclosed in Example 3.)

Development of BaculoG/PCV3 ORF2, P9; live, adjuvanted with 20% Carbopol® vaccine (methods used to develop the vaccine are disclosed in Example 4.)

Development of the control—BaculoG/no insert, P4; live, adjuvanted with 20% Carbopol® vaccine (methods used to develop the vaccine are disclosed in Example 5.)

Development of BaculoG/PCV3 ORF2, P9; killed, adjuvanted with 50% ISA 207VG vaccine (methods used to develop the vaccine are disclosed in Example 3.)

Development of BaculoG/PCV3 ORF2, P9; killed, adjuvanted with 20% Carbopol® vaccine (methods used to develop the vaccine are disclosed in Example 4.)

Development of the control—BaculoG/no insert, P4; killed, adjuvanted with 20% Carbopol® vaccine (methods used to develop the vaccine are disclosed in Example 5.)

Efficacy of the vaccines may be tested using PCV3 whole virus and PCR positive tissue (low count). Homogenates from the tissues may be generated and sequenced. The homogenates and/or the whole virus may be used to challenge vaccinated animals.

For example, in order to test the efficacy of the vaccines, PCV3 whole virus and PCR positive tissue (low count) were provided. Homogenates from the tissues were generated and sequenced. The homogenates and whole virus were used to challenge vaccinated animals.

The PCV3 recombinant ORF2 protein subunit vaccine and/or an immunogenic composition of the instant disclosure may be produced using a method of WO 2006/072065, Example 1, modified to express PCV3 ORF2 protein (rather than PCV2 ORF2 protein).

The PCV3 ORF2 coding sequence may be amplified by polymerase chain reaction (PCR) from PCV3 genomic DNA and/or a synthetically synthesized PCV3 ORF2. Restriction sites may be used to insert the desired coding sequence into a transfer vector. For example, in some embodiments, an amplified PCV3 ORF2 coding sequence may include a Kozak consensus sequence (see, e.g., Kozak M (October 1987) Nucleic Acids Res. 15 (20): 8125-8148) directly 5′ of the start codon along with flanking restriction enzyme sites.

In some embodiments, the amplified PCV3 ORF2 coding sequence may be subcloned into a baculovirus transfer vector utilizing the flanking restriction sites to generate the desired transfer vector. For example, the amplified PCV3 ORF2 coding sequence may be subcloned into a baculovirus transfer vector utilizing the flanking restriction sites to generate transfer vectors such as pVL1392-PCV3 ORF2 or pVL1393-PCV3 ORF2. Other transfer vectors commonly known in the art may be used. Recombinant baculovirus may be generated by co-transfection of insect cells with a transfer vector and baculovirus DNA. Baculovirus DNA used may include linearized and/or circular baculovirus DNA. For example, in an embodiment, recombinant baculovirus may be generated by co-transfection of Sf9 (Spodoptera frugiperda) insect cells with a transfer vector (e.g., such as pVL1392-PCV3 ORF2 and/or pVL1393-PCV3) and linearized BaculoGold™ baculovirus DNA. The linearized baculovirus DNA may be derived from Autographa californica nuclear polyhedrosis virus (AcNPV) and may contain a lethal deletion in the polyhedrin locus, therefore, rescue of viable baculovirus may be generated upon co-transfection with a transfer vector, such as pVL1392-PCV3 ORF2 and/or pVL1393-PCV3 ORF2. The resulting recombinant baculovirus may include a PCV3 ORF2 coding sequence under control of the baculovirus polyhedrin promoter. The recombinant baculovirus may be amplified on Sf9 insect cells and subsequently purified by limiting dilution cloning on Sf9 insect cells. In some embodiments, a full length circular baculovirus DNA such as Bac-to-Bac may be used. For example, Bac-to-Bac may uses transposon-mediated recombination to insert a gene of interest into a polyhedron locus. Other methods known in the art may also be used. In some embodiments, a method may be chosen based on the potential stability of the method during commercialization. For example, baculoviruses that confer increased stability in the vaccine may be selected.

In some embodiments, after seeding flasks with of a master cell culture, the flasks may be incubated at a predetermined temperature and for a specific time frame. For example, a culture may be incubated at 27° C. for four hours. Each flask may then be seeded with a recombinant baculovirus containing the PCV3 ORF2 gene. For example, a pVL1392 plasmid containing a PCV3 ORF2 gene can be co-transfected with BaculoGold® (BD Biosciences Pharmingen) baculovirus DNA into Sf+ insect cells (Protein Sciences, Meriden, CT) to generate a recombinant baculovirus containing a PCV3 ORF2 gene. The recombinant baculovirus containing the PCV3 ORF2 gene may be plaque-purified and Master Seed Virus (MSV) propagated on the SF+ cell line, aliquotted, and stored at −70° C. The MSV may be positively identified as PCV3 ORF2 baculovirus by PCR-RFLP using baculovirus specific primers. Insect cells infected with PCV3 ORF2 baculovirus to generate MSV or Working Seed Virus may express PCV3 ORF2 antigen as detected by polyclonal serum or monoclonal antibodies in an indirect fluorescent antibody assay. Additionally, the identity of the PCV3 ORF2 baculovirus may be confirmed by N-terminal amino acid sequencing. The PCV3 ORF2 baculovirus MSV is also tested for purity in accordance with 9 C.F.R. Sections 113.27 (c), 113.28, and 113.55. Each recombinant baculovirus seeded into the spinner flasks may have varying multiplicities of infection (MOIs).

After being seeded with the baculovirus, the flasks may be incubated at 27±2° C. for 7 days and may also be agitated at 100 rpm during that time. The flasks may use ventilated caps to allow for air flow. Samples from each flask may be taken every 24 hours for the next 7 days. After extraction, each sample may be centrifuged, and both the pellet and the supernatant are separated and then microfiltered through a 0.45-1.0 μm pore size membrane.

The amount of ORF3 in the resulting samples may then be quantified via an ELISA assay. The ELISA assay may be conducted with an anti-PCV3 antibody diluted to 1:6000 in 0.05M Carbonate buffer (pH 9.6). 100 μL of the antibody may then be placed in the wells of the microtiter plate, sealed, and incubated overnight at 37° C. The plate is then washed three times with a wash solution which comprised 0.5 mL of Tween 20 (Sigma, St. Louis, MO), 100 mL of 1OX D-PBS (Gibco Invitrogen, Carlsbad, CA) and 899.5 mL of distilled water. Subsequently, 250 μL of a blocking solution (5 g Carnation Non-fat dry milk (Nestle, Glendale, CA) in 10 mL of D-PBS QS to 100 mL with distilled water) is added to each of the wells. The next step is to wash the test plate and then add pre-diluted antigen. The pre-diluted antigen is produced by adding 200 μL of diluent solution (0.5 mL Tween 20 in 999.5 mL D-PBS) to each of the wells on a dilution plate. The sample is then diluted at a 1:240 ratio and a 1:480 ratio, and 100 μL of each of these diluted samples is then added to one of the top wells on the dilution plate (i.e. one top well received 100 μL of the 1:240 dilution and the other received 100 μL of the 1:480 dilution). Serial dilutions may then be done for the remainder of the plate by removing 100 μL from each successive well and transferring it to the next well on the plate. Each well is mixed prior to doing the next transfer. The test plate washing includes washing the plate three times with the wash buffer. The plate is then sealed and incubated for an hour at 37° C. before being washed three more times with the wash buffer. The detection antibody used is an antibody to PCV ORF2. It is diluted to 1 to 300 in diluent solution, and 100 μL of the diluted detection antibody was then added to the wells. The plate is then scaled and incubated for an hour at 37° C. before being washed three times with the wash buffer. Conjugate diluent is then prepared by adding normal rabbit serum (Jackson Immunoresearch, West Grove, PA) to the diluent solution to 1% concentration.

Conjugate antibody Goat anti-mouse (H+1)-HRP (Jackson Immunoresearch) is diluted in the conjugate diluent to 1:10,000. 100 μL of the diluted conjugate antibody is then added to each of the wells. The plate is then sealed and incubated for 45 minutes at 37° C. before being washed three times with the wash buffer. 100 μL of substrate (TMB Peroxidase Substrate, Kirkgaard and Perry Laboratories (KPL), Gaithersburg, MD), mixed with an equal volume of Peroxidase Substrate B (KPL) is added to each of the wells. The plate is incubated at room temperature for 15 minutes. 100 μL of IN HCL solution is then added to all of the wells to stop the reaction. The plate is then run through an ELISA reader.

Advantageous insect cells can be cultured, and the PCV3 ORF2 protein produced, under serum-free conditions; such as the serum-free insect cells of U.S. Pat. No. 6,103,526 (expresSF+ cell line).

The adjuvants, cell culture supernatants, preservatives, stabilizing agents, viral vectors, immunomodulatory agents and dosages disclosed in U.S. Pat. Nos. 9,610,345 and 9,669,087 are contemplated, both incorporated herein by reference.

The immunogenic composition as used herein is effective for inducing an immune response against PCV3 and preventing, reducing and/or lessening the severity of the clinical symptoms associated with PCV3 infection. The composition generally comprises at least one PCV3 antigen.

PCV3 in pigs may exhibit a wide variety of symptoms and in many cases individual animals exhibit only a small subset of the potential symptoms. Symptoms associated with the presence of PCV3 include viremia, virus shedding, for example, the presence of viral nucleic acids in emissions from the body such as colostrum, milk, feces, saliva, and eye swabs. For example, Jiang et al., “Induction of porcine dermatitis and nephropathy syndrome in piglets by infection with porcine circovirus type 3”, J. Virol. doi: 10.1128/JVI.02045-18, the disclosure of which is incorporated by reference, relates to inoculating piglets with PCV3 and observing resultant clinical signs. The present disclosure relates to treating and/or reducing symptoms of porcine dermatitis and nephropathy syndrome (PDNS)-like disease, lymphocytic dysplasia and necrosis caused by PCV3 by administering a composition of the disclosure.

The mere presence of antibodies, especially in young pigs or piglets, e.g., pigs or piglets of less than 15 weeks of age, such as less than 10 weeks of age, for instance, less than 6 weeks of age, for instance, less than 3, 2 or 1 week of age or at birth, may not be indicative of exposure to PCV3 and/or disease. Pigs or piglets that have had exposure and/or have antibodies against PCV3 can still enjoy benefits of compositions of the disclosure, e.g., by reducing or preventing or lessening severity of symptoms.

Thus, the compositions of the disclosure can be used in methods for eliciting an immune response, which can be a protective immune response, as well as methods for reducing or preventing or lessening severity of symptoms and, the dosages, formulations and the like for reducing or preventing or lessening severity of symptoms are as for methods for eliciting an immune response. Thus, herein where methods are described as to eliciting an immune response, these methods can be practiced for reducing or preventing or lessening severity of symptoms; and compositions described herein, which are useful for eliciting an immune response, are likewise useful for and compositions for reducing or preventing or lessening severity of symptoms (as well as being compositions for eliciting an immune response).

Unless defined otherwise, all technical and scientific terms used herein have the same meanings as commonly understood by one of ordinary skill in the art to which this disclosure belongs. The term “immunogenic composition” as used herein refers to any pharmaceutical composition containing a PCV3 antigen, which composition can be used to prevent or treat a PCV3 infection-associated disease or condition in a subject. A preferred immunogenic composition can induce, stimulate or enhance the immune response against PCV3. The term thus encompasses both subunit immunogenic compositions, as described below, as well as compositions containing whole killed, or attenuated and/or inactivated PCV3.

The term “subunit immunogenic composition” as used herein refers to a composition containing at least one immunogenic polypeptide or antigen, but not all antigens, derived from or homologous to an antigen from PCV3. Such a composition is substantially free of intact PCV3. Thus, a “subunit immunogenic composition” is prepared from at least partially purified or fractionated (preferably substantially purified) immunogenic polypeptides from PCV3, or recombinant analogs thereof. A subunit immunogenic composition can comprise the subunit antigen or antigens of interest substantially free of other antigens or polypeptides from PCV3, or in fractionated form. A preferred immunogenic subunit composition comprises the PCV3 ORF2 protein as described below.

An “immunological or immune response” to a composition or vaccine is the development in the host of a cellular and/or antibody-mediated immune response to the composition or vaccine of interest. Usually, an “immune response” includes but is not limited to one or more of the following effects: the production or activation of antibodies, B cells, helper T cells, suppressor T cells, and/or cytotoxic T cells and/or γδ T cells, directed specifically to an antigen or antigens included in the composition or vaccine of interest. Preferably, the host will display either a therapeutic or protective immunological response such that resistance to new infection will be enhanced and/or the clinical severity of the disease reduced. Such protection will be demonstrated by either a reduction in number or severity of, or lack of one or more of the symptoms associated with PCV3 infections as described above.

The terms “immunogenic” protein or polypeptide or “antigen” as used herein refer to an amino acid sequence which elicits an immunological response as described above. An “immunogenic” protein or polypeptide, as used herein, includes the full-length sequence of any PCV3 proteins, analogs thereof, or immunogenic fragments thereof. The term “immunogenic fragment” refers to a fragment of a protein, which includes one or more epitopes and thus elicits the immunological response described above. Such fragments can be identified using any number of epitope mapping techniques, well known in the art. Sec, e.g., Epitope Mapping Protocols in Methods in Molecular Biology, Vol. 66 (Glenn E. Morris, Ed., 1996) Humana Press, Totowa, N.J. For example, linear epitopes may be determined by e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871; Geysen et al. (1984) Proc. Natl. Acad. Sci. USA 81:3998-4002; Geysen et al. (1986) Molec. Immunol. 23:709-715, all incorporated herein by reference. Similarly, conformational epitopes are readily identified by determining spatial conformation of amino acids such as by, e.g., x-ray crystallography and 2-dimensional nuclear magnetic resonance. See, e.g., Epitope Mapping Protocols, supra.

Synthetic antigens are also included within the definition, for example, polyepitopes, flanking epitopes, and other recombinant or synthetically derived antigens. Sec, e.g., Bergmann et al. (1993) Eur. J. Immunol. 23:2777-2781; Bergmann et al. (1996), J. Immunol. 157:3242-3249; Suhrbier, A. (1997), Immunol. and Cell Biol. 75:402-408; Gardner et al., (1998) 12th World AIDS Conference, Geneva, Switzerland, Jun. 28-Jul. 3, 1998.

In a preferred embodiment of the present disclosure, an immunogenic composition that induces an immune response and, more preferably, confers protective immunity against the clinical signs of PCV3 infection, is provided. The composition most preferably comprises the polypeptide, or a fragment thereof, expressed by ORF2 of PCV3, as the antigenic component of the composition. PCV3 ORF2 DNA and protein, used herein for the preparation of the compositions and within the processes provided herein is a highly conserved domain within PCV3 isolates and thereby, any PCV3 ORF2 would be effective as the source of the PCV3 ORF2 DNA and/or polypeptide as used herein. A preferred PCV3 ORF2 protein translated from the nucleotide sequence of SEQ ID NO. 1. A preferred PCV3 ORF2 polypeptide is provided herein, but it is understood by those of skill in the art that this sequence could vary by as much as 6-10% in sequence homology and still retain the antigenic characteristics that render it useful in immunogenic compositions. Moreover, the antigenic characteristic of a modified antigen is still retained, when the modified antigen confers at least 30%, at least 40%, at least 50%, at least 60%, at least 70%, at least 80%, at least 90% or at least 100% of the protective immunity as compared to the PCV3 ORF2 protein, encoded by the polynucleotide sequence of SEQ ID NO: 1. An “immunogenic composition” as used herein, means a PCV3 ORF2 protein which elicits an “immunological response” in the host of a cellular and/or antibody-mediated immune response to PCV3 ORF2 protein. Preferably, this immunogenic composition is capable of eliciting or enhancing an immune response against PCV3 thereby conferring protective immunity against PCV3 infection and a reduction in the incidence of, severity of, or prevention of one or more, and preferably all of the clinical signs associated therewith.

In some forms, immunogenic portions of PCV3 ORF2 protein are used as the antigenic component in the composition. The term “immunogenic portion” as used herein refers to truncated and/or substituted forms, or fragments of PCV3 ORF2 protein and/or polynucleotide, respectively. Preferably, such truncated and/or substituted forms, or fragments will comprise at least 6 contiguous amino acids from the full-length ORF2 polypeptide. More preferably, the truncated or substituted forms, or fragments will have at least 10, more preferably at least 15, and still more preferably at least 19 contiguous amino acids from the full-length ORF2 polypeptide. It is further understood that such sequences may be a part of larger fragments or truncated forms.

A further preferred PCV3 ORF2 polypeptide provided herein is encoded by the nucleotide sequence of SEQ ID NO: 1. However, it is understood by those of skill in the art that this sequence could vary by as much as 6-20% in sequence homology and still retain the antigenic characteristics that render it useful in immunogenic compositions. In some forms, a truncated or substituted form, or fragment of this PVC3 ORF2 polypeptide is used as the antigenic component in the composition. Preferably, such truncated or substituted forms, or fragments will comprise at least 18 contiguous nucleotides from the full-length ORF2 nucleotide sequence. More preferably, the truncated or substituted forms, or fragments, will have at least 30, more preferably at least 45, and still more preferably at least 57 contiguous nucleotides of the full-length ORF2 nucleotide sequence, e.g. SEQ ID NO: 1.

“Sequence Identity” as it is known in the art refers to a relationship between two or more polypeptide sequences or two or more polynucleotide sequences, namely a reference sequence and a given sequence to be compared with the reference sequence. Sequence identity is determined by comparing the given sequence to the reference sequence after the sequences have been optimally aligned to produce the highest degree of sequence similarity, as determined by the match between strings of such sequences. Upon such alignment, sequence identity is ascertained on a position-by-position basis, e.g., the sequences are “identical” at a particular position if at that position, the nucleotides or amino acid residues are identical. The total number of such position identities is then divided by the total number of nucleotides or residues in the reference sequence to give % sequence identity. Sequence identity can be readily calculated by known methods, including but not limited to, those described in Computational Molecular Biology, Lesk, A. N., ed., Oxford University Press, New York (1988), Biocomputing: Informatics and Genome Projects, Smith, D. W., ed., Academic Press, New York (1993); Computer Analysis of Sequence Data, Part I, Griffin, A. M., and Griffin, H. G., eds., Humana Press, New Jersey (1994); Sequence Analysis in Molecular Biology, von Heinge, G., Academic Press (1987); Sequence Analysis Primer, Gribskov, M. and Devereux, J., eds., M. Stockton Press, New York (1991); and Carillo, H., and Lipman, D., SIAM J. Applied Math., 48: 1073 (1988), the teachings of which are incorporated herein by reference. Preferred methods to determine the sequence identity are designed to give the largest match between the sequences tested. Methods to determine sequence identity are codified in publicly available computer programs which determine sequence identity between given sequences. Examples of such programs include, but are not limited to, the GCG program package (Devereux, J., et al., Nucleic Acids Research, 12(1):387 (1984)), BLASTP, BLASTN and FAS TA (Altschul, S. F. et al., J. Molec. Biol., 215:403-410 (1990). The BLASTX program is publicly available from NCBI and other sources (BLAST Manual, Altschul, S. et al., NCVI NLM NIH Bethesda, Md. 20894, Altschul, S. F. et al., J. Molec. Biol., 215:403-410 (1990), the teachings of which are incorporated herein by reference). These programs optimally align sequences using default gap weights in order to produce the highest level of sequence identity between the given and reference sequences. As an illustration, by a polynucleotide having a nucleotide sequence having at least, for example, 85%, preferably 90%, even more preferably 95% “sequence identity” to a reference nucleotide sequence, it is intended that the nucleotide sequence of the given polynucleotide is identical to the reference sequence except that the given polynucleotide sequence may include up to 15, preferably up to 10, even more preferably up to 5 point mutations per each 100 nucleotides of the reference nucleotide sequence. In other words, in a polynucleotide having a nucleotide sequence having at least 85%, preferably 90%, even more preferably 95% identity relative to the reference nucleotide sequence, up to 15%, preferably 10%, even more preferably 5% of the nucleotides in the reference sequence may be deleted or substituted with another nucleotide, or a number of nucleotides up to 15%, preferably 10%, even more preferably 5% of the total nucleotides in the reference sequence may be inserted into the reference sequence. These mutations of the reference sequence may occur at the 5 or 3′ terminal positions of the reference nucleotide sequence or anywhere between those terminal positions, interspersed either individually among nucleotides in the reference sequence or in one or more contiguous groups within the reference sequence. Analogously, by a polypeptide having a given amino acid sequence having at least, for example, 85%, preferably 90%, even more preferably 95% sequence identity to a reference amino acid sequence, it is intended that the given amino acid sequence of the polypeptide is identical to the reference sequence except that the given polypeptide sequence may include up to 15, preferably up to 10, even more preferably up to 5 amino acid alterations per each 100 amino acids of the reference amino acid sequence. In other words, to obtain a given polypeptide sequence having at least 85%, preferably 90%, even more preferably 95% sequence identity with a reference amino acid sequence, up to 15%, preferably up to 10%, even more preferably up to 5% of the amino acid residues in the reference sequence may be deleted or substituted with another amino acid, or a number of amino acids up to 15%, preferably up to 10%, even more preferably up to 5% of the total number of amino acid residues in the reference sequence may be inserted into the reference sequence. These alterations of the reference sequence may occur at the amino or the carboxy terminal positions of the reference amino acid sequence or anywhere between those terminal positions, interspersed either individually among residues in the reference sequence or in the one or more contiguous groups within the reference sequence. Preferably, residue positions which are not identical differ by conservative amino acid substitutions. However, conservative substitutions are not included as a match when determining sequence identity.

“Sequence homology”, as used herein, refers to a method of determining the relatedness of two sequences. To determine sequence homology, two or more sequences are optimally aligned, and gaps are introduced if necessary. However, in contrast to “sequence identity”, conservative amino acid substitutions are counted as a match when determining sequence homology. In other words, to obtain a polypeptide or polynucleotide having 95% sequence homology with a reference sequence, 85%, preferably 90%, even more preferably 95% of the amino acid residues or nucleotides in the reference sequence must match or comprise a conservative substitution with another amino acid or nucleotide, or a number of amino acids or nucleotides up to 15%, preferably up to 10%, even more preferably up to 5% of the total amino acid residues or nucleotides, not including conservative substitutions, in the reference sequence may be inserted into the reference sequence. Preferably the homolog sequence comprises at least a stretch of 50, even more preferably at least 100, even more preferably at least 250, and even more preferably at least 500 nucleotides.

A “conservative substitution” refers to the substitution of an amino acid residue or nucleotide with another amino acid residue or nucleotide having similar characteristics or properties including size, hydrophobicity, etc., such that the overall functionality does not change significantly.

“Isolated” means altered “by the hand of man” from its natural state, i.e., if it occurs in nature, it has been changed or removed from its original environment, or both. For example, a polynucleotide or polypeptide naturally present in a living organism is not “isolated,” but the same polynucleotide or polypeptide separated from the coexisting materials of its natural state is “isolated”, as the term is employed herein.

Thus, the immunogenic composition as used herein also refers to a composition that comprises PCV3 ORF2 protein, wherein said PCV3 ORF2 protein is anyone of those, described above.

According to a further aspect, PCV3 ORF2 protein is provided in the immunological composition at an antigen inclusion level effective for inducing the desired immune response, namely reducing the incidence of, lessening the severity of, or preventing one or more clinical signs resulting from PCV3 infection. Preferably, the PCV3 ORF2 protein inclusion level is at least 0.2 μg antigen/ml of the final immunogenic composition (μg/ml), more preferably from about 0.2 to about 400 μg/ml, still more preferably from about 0.3 to about 200 μg/ml, even more preferably from about 0.35 to about 100 μg/ml, still more preferably from about 0.4 to about 50 g/ml, still more preferably from about 0.45 to about 30 μg/ml, still more preferably from about 0.6 to about 15 μg/ml, even more preferably from about 0.75 to about 8 μg/ml, even more preferably from about 1.0 to about 6 μg/ml, still more preferably from about 1.3 to about 3.0 μg/ml, even more preferably from about 1.4 to about 2.5 μg/ml, even more preferably from about 1.5 to about 2.0 μg/ml, and most preferably about 1.6 μg/ml.

According to a further aspect, the ORF2 antigen inclusion level is at least 0.2 μg PCV3 ORF2 protein as described above per dose of the final antigenic composition (μg/dose), more preferably from about 0.2 to about 400 μg/dose, still more preferably from about 0.3 to about 200 μg/dose, even more preferably from about 0.35 to about 100 μg/dose, still more preferably from about 0.4 to about 50 μg/dose, still more preferably from about 0.45 to about 30 μg/dose, still more preferably from about 0.6 to about 15 μg/dose, even more preferably from about 0.75 to about 8 μg/dose, even more preferably from about 1.0 to about 6 μg/dose, still more preferably from about 1.3 to about 3.0 μg/dose, even more preferably from about 1.4 to about 2.5 μg/dose, even more preferably from about 1.5 to about 2.0 μg/dose, and most preferably about 1.6 μg/dose. In an embodiment, ORF2 antigen (e.g., PCV3 ORF2 protein) may be present in a dose of the final composition in a range from about 1.3 to about 3 μg. For example, the final antigenic composition may include about 1.6 μg of PCV3 ORF2 protein in a 1 mL dose.

The PCV3 ORF2 polypeptide used in the immunogenic composition in accordance with the present disclosure can be derived in any fashion including isolation and purification of PCV3 ORF2, standard protein synthesis, and recombinant methodology. Preferred methods for obtaining PCV3 ORF2 polypeptide are provided in U.S. patent application Ser. No. 11/034,797, the teachings and content of which are hereby incorporated by reference. Briefly, susceptible cells are infected with a recombinant viral vector containing PCV3 ORF2 DNA coding sequences, PCV3 ORF2 polypeptide is expressed by the recombinant virus, and the expressed PCV3 ORF2 polypeptide is recovered from the supernate by filtration and inactivated by any conventional method, preferably using binary ethylenimine, which is then neutralized to stop the inactivation process.

The immunogenic composition as used herein also refers to a composition that comprises i) any of the PCV3 ORF2 protein described above, preferably in concentrations described above, and ii) at least a portion of the viral vector expressing said PCV3 ORF2 protein, preferably of a recombinant baculovirus. Moreover, the immunogenic composition can comprise any of the PCV3 ORF2 proteins described above, preferably in concentrations described above, ii) at least a portion of the viral vector expressing said PCV3 ORF2 protein, preferably of a recombinant baculovirus, and iii) a portion of the cell culture supernatant.

The immunogenic composition as used herein also refers to a composition that comprises i) any of the PCV3 ORF2 proteins described above, preferably in concentrations described above, ii) at least a portion of the viral vector expressing said PCV3 ORF2 protein, preferably of a recombinant baculovirus, and iii) a portion of the cell culture; wherein about 90% of the components may have a size smaller than 1 μm.

The immunogenic composition as used herein also refers to a composition that comprises i) any of the PCV3 ORF2 proteins described above, preferably in concentrations described above, ii) at least a portion of the viral vector expressing said PCV3 ORF2 protein, iii) a portion of the cell culture, iv) and inactivating agent to inactivate the recombinant viral vector preferably BEI, wherein about 90% of the components i) to iii) may have a size smaller than 1 μm. Preferably, BEI is present in concentrations effective to inactivate the baculovirus. Effective concentrations are described above.

The immunogenic composition as used herein also refers to a composition that comprises i) any of the PCV3 ORF2 proteins described above, preferably in concentrations described above, ii) at least a portion of the viral vector expressing said PCV3 ORF2 protein, iii) a portion of the cell culture, iv) an inactivating agent to inactivate the recombinant viral vector preferably BEI, and v) an neutralization agent to stop the inactivation mediated by the inactivating agent, wherein about 90% of the components i) to iii) may have a size smaller than 1 μm. Preferably, if the inactivating agent is BEI, said composition comprises sodium thiosulfate in equivalent amounts to BEI.

The polypeptide is incorporated into a composition that can be administered to an animal susceptible to PCV3 infection. In preferred forms, the composition may also include additional components known to those of skill in the art (see also Remington's Pharmaceutical Sciences. (1990). 18th ed. Mack Publ., Easton). Additionally, the composition may include one or more veterinary-acceptable carriers. As used herein, “a veterinary-acceptable carrier” includes any and all solvents, dispersion media, coatings, adjuvants, stabilizing agents, diluents, preservatives, antibacterial and antifungal agents, isotonic agents, adsorption delaying agents, and the like. In a preferred embodiment, the immunogenic composition comprises PCV3 ORF2 protein as provided herewith, preferably in concentrations described above, which is mixed with an adjuvant, preferably Carbopol®, and physiological saline.

Those of skill in the art will understand that the composition used herein may incorporate known injectable, physiologically acceptable sterile solutions. For preparing a ready-to-use solution for parenteral injection or infusion, aqueous isotonic solutions, such as e.g. saline or corresponding plasma protein solutions, are readily available. In addition, the immunogenic and vaccine compositions of the present disclosure can include diluents, isotonic agents, stabilizers, or adjuvants. Diluents can include water, saline, dextrose, ethanol, glycerol, and the like. Isotonic agents can include sodium chloride, dextrose, mannitol, sorbitol, and lactose, among others. Stabilizers include albumin and alkali salts of ethylendiamintetracetic acid, among others.

“Adjuvants” as used herein, can include aluminum hydroxide and aluminum phosphate, saponins e.g., Quil A®, QS-21® (Cambridge Biotech Inc., Cambridge Mass.), GPI-0100 (Galenica Pharmaceuticals, Inc., Birmingham, Ala.), water-in-oil emulsion, oil-in-water emulsion, water-in-oil-in-water emulsion. The emulsion can be based in particular on light liquid paraffin oil (European Pharmacopea type); isoprenoid oil such as squalane or squalene oil resulting from theoligomerization of alkenes, in particular of isobutene or decene; esters of acids or of alcohols containing a linear alkyl group, more particularly plant oils, ethyl oleate, propylene glycol di-(caprylate/caprate), glyceryl tri-(caprylate/caprate) or propylene glycol dioleate; esters of branched fatty acids or alcohols, in particular isostearic acid esters. The oil is used in combination with emulsifiers to form the emulsion. The emulsifiers are preferably nonionic surfactants, in particular esters of sorbitan, of mannide (e.g. anhydromannitol oleate), of glycol, of polyglycerol, of propylene glycol and of oleic, isostearic, ricinoleic or hydroxystearic acid, which are optionally ethoxylated, and polyoxypropylene-polyoxyethylene copolymer blocks, in particular the Pluronic® products, especially L121. See Hunter et al., The Theory and Practical Application of Adjuvants (Ed.Stewart-Tull, D. E. S.). John Wiley and Sons, NY, pp 51-94 (1995) and Todd et al., Vaccine 15:564-570 (1997).

For example, it is possible to use the SPT emulsion described on page 147 of “Vaccine Design, The Subunit and Adjuvant Approach” edited by M. Powell and M. Newman, Plenum Press, 1995, and the emulsion MF59 described on page 183 of this same book.

A further instance of an adjuvant is a compound chosen from the polymers of acrylic or methacrylic acid and the copolymers of maleic anhydride and alkenyl derivative. Advantageous adjuvant compounds are the polymers of acrylic or methacrylic acid, which are cross-linked, especially with polyalkenyl ethers of sugars or polyalcohols. These compounds are known by the term carbomer (Phameuropa Vol. 8, No. 2, June 1996). Persons skilled in the art can also refer to U.S. Pat. No. 2,909,462 which describes such acrylic polymers cross-linked with a polyhydroxylated compound having at least 3 hydroxyl groups, preferably not more than 8, the hydrogen atoms of at least three hydroxyls being replaced by unsaturated aliphatic radicals having at least 2 carbon atoms. The preferred radicals are those containing from 2 to 4 carbon atoms, e.g. vinyls, allyls and other ethylenically unsaturated groups. The unsaturated radicals may themselves contain other substituents, such as methyl. The products sold under the name Carbopol®; (BF Goodrich, Ohio, USA) are particularly appropriate. They are cross-linked with an allyl sucrose or with allyl pentaerythritol. Among them, there may be mentioned Carbopol® 974P, 934P and 971P. Most preferred is the use of Carbopol®, in particular the use of Carbopol® 971P, preferably in amounts of about 500 μg to about 5 mg per dose, even more preferred in an amount of about 750 μg to about 2.5 mg per dose and most preferred in an amount of about 1 mg per dose. In particular, a dose of the final composition may include Carbopol® or Carbopol® 971 in a range from about 750 μg to about 2.5 mg Carbopol®. For example, in some embodiments a dose of the final composition may include about 1 mg of Carbopol® 971.

Further suitable adjuvants include, but are not limited to, the RIBI® adjuvant system (Ribi Inc.), Block co-polymer (CytRx, Atlanta Ga.), SAF-M (Chiron, Emeryville Calif.), monophosphoryl lipid A, Avridine lipid-amine adjuvant, heat-labile enterotoxin from E. coli (recombinant or otherwise), cholera toxin, MONTANIDE™ IMS 1314, or muramyl dipeptide among many others.

Preferably, the adjuvant is added in an amount of about 100 μg to about 10 mg per dose. Even more preferably, the adjuvant is added in an amount of about 100 μg to about 10 mg per dose. Even more preferably, the adjuvant is added in an amount of about 500 μg to about 5 mg per dose. Even more preferably, the adjuvant is added in an amount of about 750 μg to about 2.5 mg per dose. Most preferably, the adjuvant is added in an amount of about 1 mg per dose.

Additionally, the composition can include one or more pharmaceutical-acceptable carriers. As used herein, “a pharmaceutical-acceptable carrier” includes any and all solvents, dispersion media, coatings, stabilizing agents, diluents, preservatives, antibacterial and antifungal agents, isotonic agents, adsorption delaying agents, and the like. Most preferably, the composition provided herewith, contains PCV3 ORF2 protein recovered from the supernate of in vitro cultured cells, wherein said cells were infected with a recombinant viral vector containing PCV3 ORF2 DNA and expressing PCV3 ORF2 protein, and wherein said cell culture was treated with about 2 to about 8 mM BEI, preferably with about 5 mM BEI to inactivate the viral vector, and an equivalent concentration of a neutralization agent, preferably sodium thiosulfate solution in a final concentration of about 2 to about 8 mM, preferably of about 5 mM.

The present disclosure also relates to an immunogenic composition that comprises i) any of the PCV3 ORF2 proteins described above, preferably in concentrations described above, i) at least a portion of the viral vector expressing said PCV3 ORF2 protein, iii) a portion of the cell culture, iv) an inactivating agent to inactivate the recombinant viral vector preferably BEI, and v) an neutralization agent to stop the inactivation mediated by the inactivating agent, preferably sodium thiosulfate in equivalent amounts to BEI; and vi) a suitable adjuvant, preferably Carbopol 971 in amounts described above; wherein about 90% of the components i) to iii) have a size smaller than 1 μm. According to a further aspect, this immunogenic composition further comprises a pharmaceutical acceptable salt, preferably a phosphate salt in physiologically acceptable concentrations. Preferably, the pH of said immunogenic composition is adjusted to a physiological pH, meaning between about 6.5 and 7.5.

In an embodiment, the immunogenic composition may refer to a composition that includes in a one ml dose i) at least some PCV3 ORF2 protein, ii) baculovirus expressing said PCV3 ORF2 protein iii) cell culture, iv) an inactivating agent (e.g, BEI) having a concentration in a range from about 2 to about 8 mM, v) a neutralization agent (e.g., sodium thiosulfate) in equivalent amounts to the inactivating agent; and vi) a predetermined amount of adjuvant (e.g., Carbopol® 971), and vii) phosphate salt in a physiologically acceptable concentration. In some embodiments, components may be selected such that 90% of the combination of components that includes the i. PCV3 ORF2 protein, ii. baculovirus that expresses the protein and iii. cell culture have a size smaller than 1 μm. Further, in some embodiments one or more components of the immunogenic composition may be selected such that the immunogenic composition has a pH in a range from about 6.5 to about 7.5. Selection of the components and/or determinations with respect to amounts and/or concentrations may relate to various factors that affect stability of the immunogenic composition, case of manufacturing, availability of materials, age, size, and/or condition of animals to be treated and/or results desired.

For example, the immunogenic composition as used herein also refers to a composition that comprises per one ml i) at least 1.6 μg of PCV3 ORF2 protein described above, ii) at least a portion of baculovirus expressing said PCV3 ORF2 protein iii) a portion of the cell culture, iv) about 2 to 8 mM BEI, v) sodium thiosulfate in equivalent amounts to BEI; and vi) about 1 mg Carbopol® 971, and vii) phosphate salt in a physiologically acceptable concentration; wherein about 90% of the components i) to iii) may have a size smaller than 1 μm and the pH of said immunogenic composition is adjusted to about 6.5 to 7.5.

The immunogenic compositions can further include one or more other immunomodulatory agents such as, e.g., interleukins, interferons, or other cytokines (such as, but not limited to, IL-1, IL-2, IL-7, IFN-alpha, IFN-beta, IFN-gamma, etc.). The immunogenic compositions can also include Gentamicin and Merthiolate. While the amounts and concentrations of adjuvants and additives useful in the context of the present disclosure can readily be determined by the skilled artisan, the present disclosure contemplates compositions comprising from about 50 μg to about 2000 μg of adjuvant. In some embodiments, it may be preferable to use adjuvants in an amount of about 250 μg of adjuvant per one milliliter dose of the vaccine composition. In some embodiments, an immunogenic composition may include antibiotics at a concentration in a range from about 1 μg/mL to about 60 μg/mL. For example, an immunogenic composition may include less than about 30 μg/ml of antibiotics.

The immunogenic composition as used herein also refers to a composition that comprises i) any of the PCV3 ORF2 proteins described above, preferably in concentrations described above, ii) at least a portion of the viral vector expressing said PCV3 ORF2 protein, iii) a portion of the cell culture, iv) an inactivating agent to inactivate the recombinant viral vector preferably BEI, and v) an neutralization agent to stop the inactivation mediated by the inactivating agent, preferably sodium thiosulfate in equivalent amounts to BEI; vi) a suitable adjuvant, preferably Carbopol® 971 in amounts described above; vii) a pharmaceutical acceptable concentration of a saline buffer, preferably of a phosphate salt, and viii) an anti-microbiological active agent; wherein about 90% of the components i) to iii) have a size smaller than 1 μm.

The composition according to the disclosure may be applied intradermally, intratracheally, or intravaginally. The composition preferably may be applied intramuscularly or intranasally, most preferably intramuscularly. In an animal body, it can prove advantageous to apply the pharmaceutical compositions as described above via an intravenous or by direct injection into target tissues. For systemic application, the intravenous, intravascular, intramuscular, intranasal, intraarterial, intraperitoneal, oral, orogastric or intrathecal routes are preferred. A more local application can be effected subcutaneously, intradermally, intracutaneously, intracardially, intralobally, intramedullarly, intrapulmonarily or directly in or near the tissue to be treated (connective-, bone-, muscle-, nerve-, epithelial tissue). Depending on the desired duration and effectiveness of the treatment, the compositions according to the disclosure may be administered once or several times, also intermittently, for instance on a daily basis for several days, weeks or months and in different dosages. A single dose as well as multiple doses are contemplated. Also contemplated are combination vaccines in with other antigens of porcine pathogens. Preferred combination compositions contain PCV3 ORF2 protein and a PPV, a PRRSV antigen, a M. hyopneumoniae antigen (supernatant or bacterin), or a PRRSV antigen and a M. hyopneumoniae antigen (supernatant or bacterin) or any combination of the foregoing with a PCV2 ORF2 protein.

In some embodiments, a dosing regimen may be developed to deliver effective amounts of PCV3 ORF2 to induce a desired effect, such as an immune response in an animal and/or their progeny. Determinations with respect to dosing regimens may be related to the desired results, components selected for use in the immunogenic composition, administration route, such as parenteral and/or subcutaneous administration, number or doses delivered, for example, a single administration or multiple doses, and/or the specific properties of the animal or animal population to be treated, for example, the age, size, and/or condition of animals. Condition of animals may refer to, for example, health status, pregnancy status, size, etc. Thus, sows and piglets may require different effective doses.

As stated above, treatment methods may be different based on the outcome desired. For example, a sow may be treated to inhibit and/or prevent conditions related to porcine circovirus or a sow may be treated to inhibit and/or prevent the negative effects of infection with porcine circovirus in her piglets.

A dosing regimen may include one or more doses of an immunogenic composition that includes a predetermined amount of PCV3 ORF2 protein. For example, the dosing regimen may include doses in a range from about 2 micrograms to about 400 micrograms of the PCV3 ORF2 protein. In an embodiment, a dosing regimen of a particular immunogenic composition may include greater than about two micrograms of PCV3 ORF2 protein. In some instances, each dose of a particular immunogenic composition many include PCV3 ORF2 protein in an amount greater than about 4 micrograms. Some dosing regimen embodiments for an immunogenic composition may include immunogenic compositions at doses of at least about 8 micrograms of PCV3 ORF2 protein. For example, some dosing regimens of the immunogenic composition as disclosed herein may be structured such that at least one dose includes greater than about 16 micrograms of the desired PCV3 ORF2 protein.

In an embodiment, a dosing regimen may be selected based on the desired expression of a specific PCV3 ORF2 protein within an animal. For example, given an immunogenic composition that includes an appropriate vector and/or expression system for pigs, it may be desired that the vector delivered in the immunogenic composition is capable of delivering PCV3 ORF2 protein in amount that is in a range from about 2 micrograms to about 400 micrograms in vivo. In an embodiment, a dosing regimen of a particular immunogenic composition is structured to deliver an amount of PCV3 ORF2 protein greater than about two micrograms to an animal. In some instances, a dosing regimen for a particular immunogenic composition is structured to deliver an amount of PCV3 ORF2 protein greater than about 4 micrograms to an animal. Some dosing regimen embodiments for an immunogenic composition are structured to deliver an amount of PCV3 ORF2 protein greater than about 8 micrograms to an animal. For example, some dosing regimens of the immunogenic composition as disclosed herein may be structured such that greater than about 16 micrograms of the desired PCV3 ORF2 protein may be delivered to an animal.

Dosing regimens may also include guidance on administration routes and/or times. For example, it may be desirable to deliver a dose of an immunogenic composition to a piglet at a specific age, in particular, at about 1 week, 2 weeks or 3 weeks of age depending on the immunogenic compositions and desired results. In some instances, piglets may be administered immunogenic compositions at an age in a range from about 7 days to about 28 days. In a dosing regimen embodiment, pigs may be administered the immunogenic composition at an age in a range from about 14 days to about 26 days. For example, an administration window for piglets may be selected in range from an age of about 16 days to about 26 days. Some dosing regimen embodiments may include administering the immunogenic composition to a piglet at an age in a range from about 18 days to about 24 days.

An immunogenic composition may include recombinant PCV3 ORF2 protein. In particular, an immunogenic composition may include recombinant PCV3 ORF2 protein expressed from baculoviruses.

Further, in some instances, the immunogenic composition that includes recombinant PCV3 ORF2 protein may be administered in combination with one or more doses of additional antigens, for example, antigens from PCV2 ORF2, PPV, PRRSV, and/or M. hyopneumoniae (“M. Hyo”). The PRRSV antigen may be an attenuated live vaccine. The M. Hyo. antigen may be a bacterin, a supernatant, or a combination of bacterin and supernatant.

Multiple doses of immunogenic compositions may be administered in a dosing regimen. For example, a dosing regimen may be made of a dose of immunogenic composition that includes recombinant PCV3 ORF2 protein and a dose of an immunogenic composition that includes a recombinant PCV2 ORF2 protein. In an instance, the doses may include approximately equivalent amounts of recombinant PCV3 ORF2 protein and PCV2 ORF2 protein. An embodiment of the dosing regimen may include doses of immunogenic compositions that include recombinant PCV3 ORF2 protein and recombinant PCV2 ORF2, both of which may be expressed using baculoviruses systems expression systems.

An embodiment of a recombinant PCV3 ORF2 immunogenic composition may include additional antigens, for example antigens such as recombinant proteins from PCV3 ORF2, as well as an attenuated live PRRSV and/or a bacterin, a supernatant, or a combination of bacterin and supernatant of M. Hyo. Some embodiments of an immunogenic composition may include baculovirus expressed recombinant proteins from PCV3 ORF2 and PCV2 ORF2, as well as antigens of PRRSV (e.g., attenuated live vaccine) and/or of M. Hyo (e.g., a bacterin and/or a supernatant). Further, in some instances, an immunogenic composition may include PCV3 ORF2 protein in combination with PCV2 ORF2 protein, an attenuated live PRRSV, and/or an M. Hyo bacterin and/or a supernatant.

Immunogenic compositions may include recombinant PCV3 ORF2 protein and recombinant PCV2 ORF2 protein. In an instance, the doses may include approximately equivalent amounts of recombinant PCV3 ORF2 protein and PCV2 ORF2 protein. An embodiment of the dosing regimen may include doses of immunogenic compositions that include recombinant PCV3 ORF2 protein and recombinant PCV2 ORF2, both of which may be expressed using baculovirus expression systems.

Some embodiments of an immunogenic composition may include baculovirus expressed recombinant proteins from PCV3 ORF2, as well as PRRSV and/or M. Hyo antigens. Further, baculovirus expressed recombinant proteins from PCV3 ORF2 and PCV2 ORF2 may be combined with antigens of PRRSV and/or M. Hyo to form an immunogenic composition. As disclosed above the additional antigens may include an attenuated live PRRSV and/or an M Hyo bacterin and/or a supernatant.

For example, an immunogenic composition may comprise recombinant PCV3 ORF2 protein and recombinant PCV2 ORF2 protein. In some instances, an immunogenic composition includes approximately equivalent amounts of recombinant PCV3 ORF2 protein and PCV2 ORF2 protein. Some embodiments of an immunogenic composition may include a combination of baculovirus expressed recombinant proteins from PCV3 ORF2 and PCV2 ORF2, as well as PRRSV and/or M. Hyo.

Dosing regimens may be used to improve the economics of swine husbandry. For example, immunogenic compositions, such as vaccines may be administered to sows and/or piglets in an effort to protect sows, piglets, or both.

In particular, vaccination of sows prior to gestation may reduce the number of mummified, stillborn and/or weak piglets at farrowing if the sows are challenged by an exposure to PCV3. Generally, PCV3 is believed to be a reproductive disease. Further, use of an inactivated baculovirus-expressed PCV3 ORF2 vaccine may reduce and/or inhibit virus replication in sows. This reduction in replication may reduce the number of mummies at farrowing for the vaccinated sows at about a rate of 4%. Such a reduction may have a significant economic impact for swine producers.

It is further claimed that, the vaccine is able to protect bred gilts and sows when challenged with PCV3 in all or two or at least one trimester during the 114 days of gestation.

It is also claimed that the vaccine is able to significantly reduce the incidence of mummies, stillborns and fetus in vaccinated gilts and sows vaccinated when challenged with PCV3 in all or two or at least one trimester during the 114 days of gestation.

A dosing regimen may include vaccinating young sows (i.e., less than or equal to 5 months of age) with at least one dose of an immunogenic composition as described herein prior to breeding. The dose of the immunogenic composition as described herein may be administered intramuscularly as a one (1) mL dose prior to breeding. In some embodiments, one or more doses of vaccine may be given to sows. For example, a first vaccine may be given and followed by a booster vaccine 21 days later and prior to breeding. In some embodiments, sows may be bred in a range from 14 days to 21 days after the booster vaccination. This time frame may allow sows to mount an immune response. Utilizing such a dosing regimen may reduce and/or inhibit the number of mummies at farrowing.

Further, use of a dosing regimen that includes administering a 1 ml dose of an immunogenic composition than includes PCV3 antigen (i.e., recombinant PCV3 ORF2) may reduce, lessen and/or inhibit lymphadenopathy, lymphoid depletion and/or multinucleated/giant histiocytes in pigs infected with PCV3.

In some embodiments, a dosing regimen for vaccinating piglets at about 3 weeks of age using a baculovirus expressed PCV3 ORF2 vaccine may reduce viral load if the piglets are subsequently challenged by PCV3. For example, an amount of replicating virus in tissues of vaccinated piglets may be reduced relative to unvaccinated piglets. Further, vaccinating piglets with a PCV3 ORF2 vaccine may reduce mortality, clinical signs, gross lesions, and/or histologic lesions in vaccinated piglets relative to unvaccinated piglets that are subsequently exposed to PCV3.

The term “immune stimulant” or “immunostimulant” as used herein, means any agent or composition that can trigger the immune response, preferably without initiating or increasing a specific immune response, for example the immune response against a specific pathogen. It is further instructed to administer the immune stimulant in a suitable dose. Advantageously, the immune stimulant is Keyhole Limpet Hemacyanin (KLH) and/or incomplete Freunds adjuvant (IFA). As used herein, the role of the immune stimulant is not of an adjuvant, but as a challenge enhancer. Advantageously, KLH is emulsified in IFA containing 1 mg KLH/mL may be administered intramuscularly two days before and two days after challenge.

According to a further consideration, a porcine circovirus type 3 (PCV3) antigenic protein is provided, wherein said protein is a functional antigenic variant of PCV3 ORF2 protein, and wherein said protein is in particular also termed “the protein of the further consideration” hereinafter.

Preferably, the protein of the further consideration is a functional antigenic variant of the PCV3 ORF2 protein encoded by SEQ ID No. 1.

In one preferred aspect, the protein of the further consideration comprises substitutions and/or extensions of PCV3 ORF2.

In another preferred aspect, the protein of the further consideration is a functional antigenic variant of the protein encoded by SEQ ID No. 1 and/or the functional antigenic variant is capable of a higher yield of virus-like particles (VLPs) than the protein encoded by SEQ ID No. 1.

Preferably, said functional antigenic variant is capable of a higher yield of VLPs than the protein encoded by SEQ ID No. 1 as determinable by Western blot analysis.

According to one preferred aspect, said functional antigenic variant has fewer positive charged amino acid residues than the protein encoded by SEQ ID No. 1.

According to another preferred aspect, said functional antigenic variant has one or more substitutions in the FG loop of the protein encoded by SEQ ID No. 1, and wherein preferably those substitutions comprise substitutions of one or more of the S residue and/or the K residues and/or the H residue of the motif SKKKH (SEQ ID NO: 14) of the FG loop of the protein encoded by SEQ ID No. 1.

According to yet another preferred aspect, said functional antigenic variant has one or more substitutions in the FG loop of the protein encoded by SEQ ID No. 1, wherein those substitutions comprise substitutions of one or more of the S residue and/or the K residues of the motif SKKKH (SEQ ID NO: 14) of the FG loop of the protein encoded by SEQ ID No. 1.

According to yet a further preferred aspect, said functional antigenic variant has one or more substitutions in the FG loop of the protein encoded by SEQ ID No. 1, wherein those substitutions comprise substitutions of the S residue or H residue and all of the K residues of the motif SKKKH (SEQ ID NO: 14) of the FG loop of the protein encoded by SEQ ID No. 1.

In still another preferred aspect, said functional antigenic variant has one or more substitutions in the FG loop of the protein encoded by SEQ ID No. 1, wherein those substitutions comprise a substitution of at least S and/or H and any K of the motif SKKKH (SEQ ID NO: 14) of the FG loop of the protein encoded by SEQ ID No. 1 with Q or P or For S.

In still a further preferred aspect. said functional antigenic variant has one or more substitutions in the FG loop of the protein encoded by SEQ ID No. 1, wherein those substitutions comprise substitution of the motif SKKK (SEQ ID NO: 11) within the motif SKKKH (SEQ ID NO: 14) of the FG loop of the protein encoded by SEQ ID No. 1 with QPFS (SEQ ID NO: 12) or substitution of the motif KKKH (SEQ ID NO: 15) within the motif SKKKH (SEQ ID NO: 14) of the FG loop of the protein encoded by SEQ ID No. 1 with QPFS (SEQ ID NO: 12).

In yet another further preferred aspect, said functional antigenic variant is encodable by all or part of SEQ ID No. 1, 2, 5, 6 or 7.

In still a further preferred aspect, said functional antigenic variant is encoded by all or part of SEQ ID No. 1, 2, 5, 6 or 7.

According to a particularly preferred aspect, said functional antigenic variant has a C terminal end that extends beyond the terminal SVL sequence of the protein encoded by SEQ ID No. 1, preferably wherein said extension is all or includes a sequence from a circoviridae virus, and preferably wherein at least a part of said extension replaces the terminal SVL sequence of the protein encoded by SEQ ID No. 1.

According to another preferred aspect, said functional antigenic variant has a C terminal end that extends beyond the terminal SVL sequence of the protein encoded by SEQ ID No. 1; and wherein said extension is from 1 to 100 amino acids long.

According to a further preferred aspect, said functional antigenic variant has a C terminal end that extends beyond the terminal SVL sequence of the protein encoded by SEQ ID No. 1; and wherein said extension is from 1 to 50 amino acids long.

According to yet a another preferred aspect, said functional antigenic variant has a C terminal end that extends beyond the terminal SVL sequence of the protein encoded by SEQ ID No. 1; and wherein said extension is from 1 to 30 amino acids long.

In one particularly preferred aspect, said functional antigenic variant has a C terminal end that extends beyond the terminal SVL sequence of the protein encoded by SEQ ID No. 1.

Preferably, said extension is from 1 to 30 amino acids long and/or said extension comprises all of the sequence VKININLTPPVATSRVPSRALPLRFGCGHR (SEQ ID NO: 16).

In a further preferred aspect, said functional antigenic variant is encodable by all or part of SEQ ID No. 1, 2, 5, 6 or 7.

In a preferred aspect, said variant protein comprises or consists of an amino acid sequence having a sequence identity and/or sequence homology of at least about 80% or at least about 85% or at least about 86% or at least about 87% or at least about at least 88% or at least about 89%, e.g., in a range from about 83% to about 89%, such as 84% or 85% or 86% or 87% or 88% or 89% sequence identity and/or sequence homology, with the sequence of SEQ ID NO: 3, 4, 8, 9 or 10, and/or wherein the protein is a recombinant protein, and wherein said protein has one or more substitutions in the FG loop.

In a preferred aspect, said variant protein comprises or consists of an amino acid sequence having a sequence identity and/or sequence homology of at least about 80% or at least about 85% or at least about 86% or at least about 87% or at least about at least 88% or at least about 89%, e.g., in a range from about 83% to about 89%, such as 84% or 85% or 86% or 87% or 88% or 89% sequence identity and/or sequence homology, with the sequence of SEQ ID NO: 3, 4, 8, 9 or 10, and/or wherein the protein is a recombinant protein, and wherein said protein has a C terminal end that extends beyond the terminal SVL sequence of the protein encoded by SEQ ID No. 1.

In a preferred aspect, said variant protein comprises an FG loop having one or more substitutions in the FG loop and further comprises a C terminal end that extends beyond the terminal SVL sequence of the protein encoded by SEQ ID No. 1, wherein the sequence of the variant protein comprises or consists of an amino acid sequence having sequence identity and/or sequence homology of at least about 80% or at least about 85% or at least about 86% or at least about 87% or at least about at least 88% or at least about 89%, e.g., in a range from about 83% to about 89%, such as 84% or 85% or 86% or 87% or 88% or 89% sequence identity and/or sequence homology, with the sequence of SEQ ID NO: 3, 4, 8, 9 or 10, and/or wherein the protein is a recombinant protein.

In another preferred aspect, the protein of the further consideration is a recombinant protein having been prepared by recombinant DNA techniques.

In still another preferred aspect, the protein of the further consideration is a baculovirus expressed protein.

Preferably, said PCV3 is any phylogenetic clade of PCV3 or combination of clades

Preferably, said PCV3 is selected from the group consisting of PCV3a and PCV3b.

In particular, said PCV3 is preferably selected from the group consisting PCV3a1, PCV3b1 and PCV3b2.

The PCV3 may also be selected from PCV3c (BMC Vet Res. 2019 Jul. 15; 15(1):244. doi: 10.1186/s12917-019-1977-7).

More particular, said PCV3 ORF2 is preferably from group a1, b1 or b2 (using the subtyping designation of Fux et al., “Full genome characterization of porcine circovirus type 3 isolates reveals the existence of two distinct groups of virus strains,” Virology Journal (2018) 15:25, DOI 10.1186/s12985-018-0929-3 (incorporated herein by reference); see, e.g., Table 4).

In a preferred aspect, said PCV3 ORF2 protein comprises or consists of an amino acid sequence encoded by a polynucleotide sequence having at least 90%, or at least 91%, or at least 92%, or at least 93%, or at least 94%, or at least 95%, or at least 96%, or at least 97%, or at least 98%, or at least 99%, or 100% sequence identity with SEQ ID NO: 1 or sequence homology with SEQ ID NO:1.

In another preferred aspect, said variant protein comprises or consists of an amino acid sequence encoded by a polynucleotide sequence having at least 90%, or at least 91%, or at least 92%, or at least 93%, or at least 94%, or at least 95%, or at least 96%, or at least 97%, or at least 98%, or at least 99%, or 100% sequence identity with SEQ ID NO:1 or sequence homology with SEQ ID NO:6.

In yet another preferred aspect, said variant protein comprises or consists of an amino acid sequence encoded by a polynucleotide sequence having at least 90%, or at least 91%, or at least 92%, or at least 93%, or at least 94%, or at least 95%, or at least 96%, or at least 97%, or at least 98%, or at least 99%, or 100% sequence identity with SEQ ID NO:1 or sequence homology with SEQ ID NO:7.

In yet a further preferred aspect, said PCV3 ORF2 protein comprises or consists of an amino acid sequence having at least 90%, or at least 91%, or at least 92%, or at least 93%, or at least 94%, or at least 95%, or at least 96%, or at least 97%, or at least 98%, or at least 99%, or 100% sequence identity with the sequence of SEQ ID NO: 3, 4, 8, 9 or 10 and/or wherein the protein is a recombinant protein.

In still another preferred aspect, said variant protein comprises or consists of an amino acid sequence having at least 90%, or at least 91%, or at least 92%, or at least 93%, or at least 94%, or at least 95%, or at least 96%, or at least 97%, or at least 98%, or at least 99%, or 100% sequence identity with the sequence of SEQ ID NO: 3, 4, 8, 9 or 10 and/or wherein the protein is a recombinant protein.

In still another preferred aspect, said variant protein comprises or consists of an amino acid sequence having at least 90%, or at least 91%, or at least 92%, or at least 93%, or at least 94%, or at least 95%, or at least 96%, or at least 97%, or at least 98%, or at least 99%, or 100% sequence identity with the sequence of SEQ ID NO: 3, 4, 8, 9 or 10, and/or wherein the protein is a recombinant protein, and wherein said protein has one or more substitutions in the FG loop.

In a preferred aspect, said variant protein comprises or consists of an amino acid sequence having at least 90%, or at least 91%, or at least 92%, or at least 93%, or at least 94%, or at least 95%, or at least 96%, or at least 97%, or at least 98%, or at least 99%, or 100% sequence identity with the sequence of SEQ ID NO: 3, 4, 8, 9 or 10 and/or wherein the protein is a recombinant protein.

In another preferred aspect, said variant protein comprises or consists of an amino acid sequence having at least 90%, or at least 91%, or at least 92%, or at least 93%, or at least 94%, or at least 95%, or at least 96%, or at least 97%, or at least 98%, or at least 99%, or 100% sequence identity with the sequence of SEQ ID NO: 3, 4, 8, 9 or 10, and/or wherein the protein is a recombinant protein, and wherein said protein has a C terminal end that extends beyond the terminal SVL sequence of the protein encoded by SEQ ID No. 1.

According to a preferred aspect, said protein is a recombinant protein from expression thereof by an expression vector, comprising a polynucleotide sequence that encodes the protein.

According to a preferred aspect, said protein is a recombinant protein from expression thereof by a baculovirus expression vector, comprising a polynucleotide sequence that encodes the protein.

In another preferred aspect, a nucleotide sequence is provided, wherein the nucleotide sequence encodes the protein of the further consideration, and wherein said nucleotide is also termed “the nucleotide sequence of the further consideration” hereinafter.

In a further preferred aspect, a vector is provided, wherein the vector comprises the nucleotide sequence of the further consideration, and wherein said vector is also termed “the vector of the further consideration” hereinafter.

Also, recombinant vector is provided, wherein the recombinant vector comprises the nucleotide sequence of the further consideration.

Further, an expression host is provided, wherein the expression host is transformed or transfected with the nucleotide sequence of the further consideration and wherein said expression host is also termed “the expression host of the further consideration” hereinafter.

Also, a baculovirus expression host is provided, wherein the baculovirus expression host is transformed or transfected with the nucleotide sequence of the further consideration, and wherein said baculovirus expression host is also termed “the baculovirus expression host of the further consideration” hereinafter.

Further, a method of preparing the protein of the further consideration is provided comprising expressing a nucleotide sequence of the further consideration.

Also, a method of preparing the protein of the further consideration is provided, wherein the method comprises expressing a vector of the further consideration.

Further, a method of preparing the protein of the further consideration is provided, wherein the method comprises expressing a recombinant vector of the further consideration.

Also, a method of preparing the protein of the further consideration is provided, wherein the method comprises culturing the expression host of the further consideration to cause expression of the protein.

Further, a method of preparing the protein of the further consideration is provided, wherein the method comprises transfecting an expression host with the nucleotide sequence of the further consideration or the vector according of the further consideration, and culturing the expression host to cause expression of the protein.

Also, a method of preparing the protein of the further consideration is provided, wherein the method comprises culturing the baculovirus expression host of the further consideration to cause expression of the protein.

Also, a method of preparing the protein of the further consideration is provided, wherein the method comprises transfecting a baculovirus expression host with the nucleotide sequence of the further consideration or the vector according of the further, and culturing the baculovirus expression host to cause expression of the protein.

Preferably, in any of the above methods of preparing the protein of the further consideration an inactivating agent is used when sufficient levels of expressed protein have been achieved and wherein the inactivating agent is preferably binary ethyleneimine (BEI) is used when sufficient levels of expressed protein have been achieved.

Preferably, any of the above methods of preparing the protein of the further consideration comprises transfecting a baculovirus expression host with the nucleotide sequence of vector and culturing the baculovirus expression host in a medium to cause expression of the protein; wherein the medium post expression of the protein comprises (i) said protein, (ii) at least a portion of baculovirus that expressed said protein, (iii) a portion of cell culture of cells that were infected or transfected with recombinant baculovirus that expressed said protein.

Preferably, any of the above methods of preparing the protein of the further consideration comprises transfecting a baculovirus expression host with the nucleotide sequence of vector and culturing the baculovirus expression host in a medium to cause expression of the protein; wherein the medium post expression of the protein comprises (i) said protein, (ii) at least a portion of baculovirus that expressed said protein, (iii) a portion of cell culture of cells that were infected or transfected with recombinant baculovirus that expressed said protein; and wherein about 90% of the components (i) to (iii) have a size smaller than 1 μm.

Preferably, any of the above methods of preparing the protein of the further consideration comprises transfecting a baculovirus expression host with the nucleotide sequence of vector and culturing the baculovirus expression host in a medium to cause expression of the protein; wherein the medium post expression of the protein comprises (i) said protein, (ii) at least a portion of baculovirus that expressed said protein, (iii) a portion of cell culture of cells that were infected or transfected with recombinant baculovirus that expressed said protein; and wherein about 90% of the components (i) to (iii) have a size smaller than 1 μm and the pH of said composition is adjusted to about 6.5 to 7.5.

Preferably, any of the above methods of preparing the protein of the further consideration comprises producing the protein by a baculovirus expression system in cultured insect cells.

Preferably, any of the above methods of preparing the protein of the further consideration comprises producing the protein by a baculovirus expression system in cultured insect cells; and wherein the method includes the step of inactivating the baculovirus.

Preferably, any of the above methods of preparing the protein of the further consideration comprises producing the protein by a baculovirus expression system in cultured insect cells; and wherein the method includes the step of inactivating the baculovirus; and wherein inactivating step comprises heat treatment or use of a virus inactivating agent.

Preferably, any of the above methods of preparing the protein of the further consideration comprises producing the protein by a baculovirus expression system in cultured insect cells; and wherein the method includes the step of inactivating the baculovirus; and wherein inactivating step comprises heat treatment or use of a virus inactivating agent; and wherein the virus inactivating agent comprises an aziridine compound.

Preferably, any of the above methods of preparing the protein of the further consideration comprises producing the protein by a baculovirus expression system in cultured insect cells; and wherein the method includes the step of inactivating the baculovirus; and wherein inactivating step comprises heat treatment or use of a virus inactivating agent; and wherein the virus inactivating agent comprises an aziridine compound; wherein the aziridine compound comprises BEI.

Further, a protein is provided, wherein said protein is obtainable by any of the above methods of preparing the protein of the further consideration.

Also, a composition is provided comprising a protein obtainable by any of the above methods of preparing the protein of the further consideration, and wherein the composition preferably comprises a carrier, diluent or excipient. Further, a composition is provided obtainable by any of the above methods of preparing the protein of the further consideration, and wherein the composition preferably comprises a carrier, diluent or excipient.

In particular, any of said compositions is also termed “the composition of the further consideration” hereinafter.

In the composition of the further consideration the protein is preferably present in an amount of 0.2 to about 400 μg/ml, or 2 to about 400 μg/ml, or 4 to about 400 μg/ml, or 8 to about 400 μg/ml, or about 0.3 to about 200 μg/ml, or 2 to about 200 μg/ml, or 4 to about 200 μg/ml, or 8 to about 200 μg/ml, or about 0.35 to about 100 μg/ml, or 2 to about 100 g/ml, or 4 to about 100 μg/ml, or 8 to about 100 μg/ml, or about 0.4 to about 50 μg/ml, or about 0.45 to about 30 μg/ml, or about 0.6 to about 15 μg/ml, or about 0.75 to about 8 μg/ml, or about 1.0 to about 6 μg/ml, or about 1.3 to about 3.0 μg/ml, or about 1.4 to about 2.5 μg/ml, or about 1.5 to about 2.0 μg/ml, or about 1.6 μg/ml.

Preferably, the composition of the further consideration comprises any one or more of a solvent, a dispersion media, a coating, a stabilizing agent, a diluent, a preservative, an anti-microbial agent, an antifungal agent, an isotonic agent, an adsorption delaying agent, an adjuvant, cell culture supernatant, a stabilizing agent, a viral vector, an expression vector, and/or an immunomodulatory agent.

Preferably, a composition of the further consideration is provided, wherein the carrier, diluent or excipient is any one or more of an adjuvant, immunomodulatory agent, cell culture supernatant, viral or expression vector or any combination thereof.

Preferably, a composition of the further consideration is provided, wherein the carrier, diluent or excipient comprises an adjuvant.

Preferably, a composition of the further consideration is provided, wherein the carrier, diluent or excipient comprises an adjuvant; wherein the adjuvant comprises one or more of a polymer of acrylic or methacrylic acid; copolymer of maleic anhydride and alkenyl derivative; a polymer of acrylic or methacrylic acid which is cross-linked; a polymer of acrylic or methacrylic acid which is cross-linked with a polyalkenyl ether of sugar or polyalcohol; a carbomer; an acrylic polymer cross-linked with a polyhydroxylated compound having at least 3 and not more than 8 hydroxyl groups with hydrogen atoms of at least three hydroxyls optionally or being replaced by unsaturated aliphatic radicals having at least 2 carbon atoms with said radicals containing from 2 to 4 carbon atoms such as vinyls, allyls and other ethylenically unsaturated groups and the unsaturated radicals may themselves contain other substituents, such as methyl; a carbopol; Carbopol® 974P; Carbopol® 934P; Carbopol® 971P; aluminum hydroxide; aluminum phosphate; a saponin; Quil A®; QS-21®; GPI-0100; a water-in-oil emulsion; an oil-in-water emulsion; a water-in-oil-in-water emulsion; an emulsion based on light liquid paraffin oil or European Pharmacopea type adjuvant; an isoprenoid oil; squalane; squalene oil resulting from oligomerization of alkenes or isobutene or decene; (an) ester(s) of acid(s) or of alcohol(s) containing a linear alkyl group; plant oil(s); ethyl oleate; propylene glycol di-(caprylate/caprate); glyceryl tri-(caprylate/caprate); propylene glycol dioleate; (an) ester(s) of branched fatty acid(s) or alcohol(s); isostearic acid ester(s); nonionic surfactant(s); (an) ester(s) of sorbitan or of mannide or of glycol or of polyglycerol or of propylene glycol or of oleic, or isostearic acid or of ricinoleic acid or of hydroxystearic acid, optionally ethoxylated, anhydromannitol oleate; polyoxypropylene-polyoxyethylene copolymer blocks, a Pluronic® product, RIBI® adjuvant system; Block co-polymer; SAF-M; monophosphoryl lipid A; Avridine lipid-amine adjuvant; heat-labile enterotoxin from E. coli (recombinant or otherwise); cholera toxin; MONTANIDE™ IMS 1314, or muramyl dipeptide.

Preferably, a composition of the further consideration is provided, wherein the carrier, diluent or excipient comprises an adjuvant; wherein the adjuvant comprises Carbopol® or Carbopol® 971.

Preferably, a composition of the further consideration is provided, wherein the carrier, diluent or excipient comprises an adjuvant; wherein the adjuvant is present in an amount from about 50 μg to about 2000 of the composition; or wherein adjuvant is present in an amount about 250 g/ml dose of the composition, or wherein the adjuvant is present in an amount of about 100 μg to about 10 mg of the composition; or wherein the adjuvant is present in an amount of about 500 μg to about 5 mg of the composition; the adjuvant is present in an amount of about 750 μg to about 2.5 mg of the composition; or the adjuvant is present in an amount of about 1 mg of the composition.

Preferably, a composition of the further consideration is provided, wherein the composition comprises an immunomodulatory agent.

Preferably, a composition of the further consideration is provided, wherein the composition comprises an immunomodulatory agent; and wherein the immunomodulatory agent is any one or more of interleukin(s), interferon(s), or other cytokine(s).

Preferably, a composition of the further consideration is provided, wherein the composition comprises an antibiotic(s).

Preferably, a composition of the further consideration is provided, wherein the composition comprises an antibiotic(s); wherein the antibiotic(s) comprise Gentamicin.

Preferably, a composition of the further consideration is provided, wherein the composition comprises an antibiotic(s); and wherein the composition comprises from about 1 μg/ml to about 60 μg/ml of antibiotic(s).

Preferably, a composition of the further consideration is provided, wherein the composition comprises an antibiotic(s); and wherein the composition comprises from about 1 μg/ml to less than about 30 μg/ml of antibiotic(s).

Preferably, a composition of the further consideration is provided, wherein the composition comprises an additional antigen.

Preferably, a composition of the further consideration is provided, wherein the composition comprises an additional antigen; wherein said additional antigen is not a PCV3 ORF2 antigen.

Preferably, a composition of the further consideration is provided, wherein the composition comprises an additional antigen; wherein said additional antigen is not a PCV3 antigen.

Preferably, a composition of the further consideration is provided, wherein the composition comprises an additional antigen of an additional porcine pathogen.

Preferably, a composition of the further consideration is provided, wherein the composition further comprises an antigen of an additional porcine pathogen, wherein said pathogen is any one or more of PCV2, PRRSV (porcine respiratory and reproductive syndrome virus) antigen, a Mycoplasma hyopneumoniae bacterin antigen, a Mycoplasma hyopneumoniae supernatant antigen, an Aujeszky's disease or pseudorabies antigen, a swine influenza antigen, a swine fever antigen (classical or African or combination thereof), an Actinobacillus pleuropneumoniae antigen, an Escherichia coli antigen, a porcine parvovirus (PPV) antigen or a Pasteurella multocida antigen.

Preferably, a composition of the further consideration is provided, wherein the composition further comprises an antigen of an additional porcine pathogen, wherein said composition further comprises one or more of an antigen of PCV2, an antigen of a PRRSV and an antigen of a PPV.

Preferably, a composition of the further consideration is provided, wherein the composition further comprises an antigen of PCV2.

Preferably, a composition of the further consideration is provided, wherein the composition further comprises an antigen of PCV2; wherein PCV2 antigen is PCV2 ORF2 protein.

Preferably, a composition of the further consideration is provided, wherein the composition further comprises an antigen of PCV2; wherein PCV2 antigen is recombinant PCV2 ORF2 protein.

Preferably, a composition of the further consideration is provided, wherein the composition further comprises an antigen of PCV2; wherein PCV2 antigen is recombinant baculovirus expressed PCV2 ORF2 protein.

Preferably, a composition of the further consideration is provided, wherein the composition is in a dosage form.

Preferably, a composition of the further consideration is provided, wherein the composition is formulated and/or packaged for a single dose or one shot administration.

Preferably, a composition of the further consideration is provided, wherein the composition is formulated and/or packaged for a multi-dose regimen.

Preferably, a composition of the further consideration is provided, wherein the composition is formulated and/or packaged for a two-dose regimen.

Preferably, a composition of the further consideration is provided, wherein the composition is in a dosage form; and wherein said dosage form is delivered from a container containing a larger amount of said composition and wherein a dosage form of said composition is capable of being delivered from said container.

Preferably, a composition of the further consideration is provided, wherein the composition is in a dosage form; and wherein said dosage form is delivered from a container containing a larger amount of said composition and wherein a dosage form of said composition is capable of being delivered from said container; and wherein said container contains at least 10 doses of said composition.

Preferably, a composition of the further consideration is provided, wherein the composition is in a dosage form; and wherein said dosage form is delivered from a container containing a larger amount of said composition and wherein a dosage form of said composition is capable of being delivered from said container; and wherein said container contains at least 50 doses of said composition.

Preferably, a composition of the further consideration is provided, wherein the composition is in a dosage form; and wherein said dosage form is delivered from a container containing a larger amount of said composition and wherein a dosage form of said composition is capable of being delivered from said container; and wherein said container contains at least 100 doses of said composition.

Preferably, a composition of the further consideration is provided, wherein the composition is in a dosage form; and wherein said dosage form is delivered from a container containing a larger amount of said composition and wherein a dosage form of said composition is capable of being delivered from said container; and wherein said container contains at least 200 doses of said composition.

Preferably, a composition of the further consideration is provided, wherein the composition is in a dosage form; and wherein said dosage form is delivered from a container containing a larger amount of said composition and wherein a dosage form of said composition is capable of being delivered from said container; and wherein said container contains at least 250 doses of said composition.

Preferably, a composition of the further consideration is provided, wherein the composition comprises an antigen of PCV2; wherein PCV2 antigen is recombinant baculovirus expressed PCV2 ORF2 protein; and wherein either the protein or combined total amount of the PCV3 ORF protein and PCV2 ORF protein are present in an amount of about 0.2 to about 400 μg/dose, or 2 to about 400 μg/dose, or 4 to about 400 μg/dose, or 8 to about 400 μg/dose, or about 0.3 to about 200 μg/dose, or 2 to about 200 μg/dose, or 4 to about 200 μg/dose, or 8 to about 200 μg/dose, or about 0.35 to about 100 μg/dose, or 2 to about 100 μg/dose, or 4 to about 100 μg/dose, or 8 to about 100 μg/dose, or about 0.4 to about 50 μg/dose, or about 0.45 to about 30 μg/dose, or about 0.6 to about 15 μg/dose, or about 0.75 to about 8 μg/dose, or about 1.0 to about 6 μg/dose, or about 1.3 to about 3.0 μg/dose, or about 1.4 to about 2.5 μg/dose, or about 1.5 to about 2.0 μg/dose, or about 1.6 μg/dose.

Preferably, a composition of the further consideration is provided, wherein the composition comprises a salt.

Preferably, a composition of the further consideration is provided, wherein the composition comprises an inactivated viral vector and/or cell culture supernate.

Preferably, a composition of the further consideration is provided, wherein the composition comprises an inactivated viral vector and cell culture supernate.

Preferably, a composition of the further consideration is provided, wherein the composition comprises (i) the protein, (ii) at least a portion of baculovirus that expressed said protein, (iii) a portion of cell culture of cells that were infected or transfected with recombinant baculovirus that expressed said protein, (iv) inactivating agent or inactivating agent comprising binary ethyleneimine (BEI), (v) sodium thiosulfate or sodium thiosulfate in equivalent amounts to inactivating agent or BEI; (vi) adjuvant or adjuvant comprising Carbopol or Carbopol 971, and (vii) phosphate salt in a physiologically acceptable concentration.

Preferably, a composition of the further consideration is provided, wherein the composition comprises (i) the protein, (ii) at least a portion of baculovirus that expressed said protein, (iii) a portion of cell culture of cells that were infected or transfected with recombinant baculovirus that expressed said protein, (iv) inactivating agent or inactivating agent comprising binary ethyleneimine (BEI), (v) sodium thiosulfate or sodium thiosulfate in equivalent amounts to inactivating agent or BEI; (vi) adjuvant or adjuvant comprising Carbopol or Carbopol 971, and (vii) phosphate salt in a physiologically acceptable concentration; and wherein the BEI is from the cell culture having been treated with about 2 to 8 or about 5 mM BEI to inactivate the baculovirus and/or the composition contains about 2 to 8 or about 5 mM BEI and/or the composition contains about 1 mg of the Carbopol or Carbopol 971.

Preferably, a composition of the further consideration is provided, wherein the composition is an immunogenic composition comprising a protein of the further consideration and a carrier, diluent or excipient.

Preferably, a composition of the further consideration is provided, wherein the composition is an immunogenic composition comprising a protein of the further consideration and a carrier, diluent or excipient; and an additional antigen as mentioned above.

Also, a process of making the composition of the further consideration is provided, wherein the protein of the further consideration is admixed with the carrier, diluent or excipient.

Further, a process of making the composition of the further consideration is provided, wherein the protein of the further consideration is admixed with the carrier, diluent or excipient; and the additional antigen.

Moreover, a protein of the further consideration is provided for use as a medicament.

Also, the protein of the further consideration or the composition of the further consideration is provided for use as a vaccine.

Also, the protein of the further consideration or the composition of the further consideration is provided for use in a method for eliciting an immune response or an immunological response or a protective immune or immunological response against PCV3 in an animal.

Also, the protein of the further consideration or the composition of the further consideration is provided for use in a method for eliciting an immune response or an immunological response or a protective immune or immunological response against PCV3 in swine.

Also, the protein of the further consideration or the composition of the further consideration is provided for use in a method for eliciting an immune response or an immunological response or a protective immune or immunological response against PCV3 in pigs.

Also, the protein of the further consideration or the composition of the further consideration is provided for use in a method for eliciting an immune response or an immunological response or a protective immune or immunological response against PCV3 in piglets.

Also, the protein of the further consideration or the composition of the further consideration is provided for use in a method for eliciting an immune response or an immunological response or a protective immune or immunological response against PCV3 in piglets; wherein the piglets are to be suckled by sows to which the protein of the further consideration or a composition of the further consideration has been administered.

Also, the protein of the further consideration or the composition of the further consideration is provided for use in a method for eliciting an immune response or an immunological response or a protective immune or immunological response against PCV3 in sows.

Also, the protein of the further consideration or the composition of the further consideration is provided for use in a method for eliciting an immune response or an immunological response or a protective immune or immunological response against PCV3 in pregnant sows, gilts or pre-breeding gilts.

Also, the protein of the further consideration or the composition of the further consideration is provided for use in inducing an immune response against PCV3 in animals.

Also, the protein of the further consideration or the composition of the further consideration is provided for use in inducing an immune response against PCV3 in swine.

Also, the protein of the further consideration or the composition of the further consideration is provided for use in inducing an immune response against PCV3 in pigs.

Also, the protein of the further consideration or the composition of the further consideration is provided for use in inducing an immune response against PCV3 in piglets.

Also, the protein of the further consideration or the composition of the further consideration is provided for use in inducing an immune response against PCV3 in piglets; wherein the piglets are to be suckled by sows to which the protein of the further consideration or a composition of the further consideration has been administered.

Also, the protein of the further consideration or the composition of the further consideration is provided for use in inducing an immune response against PCV3 in sows.

Also, the protein of the further consideration or the composition of the further consideration is provided for use in inducing an immune response against PCV3 in pregnant sows, gilts or pre-breeding gilts.

Also, the protein of the further consideration or the composition of the further consideration is provided for use in a method of reducing or preventing the clinical signs or clinical symptoms or disease caused by an infection with PCV3 in an animal or for use in a method of treating or preventing an infection with PCV3 in an animal.

Also, the protein of the further consideration or the composition of the further consideration is provided for use in a method of reducing or preventing the clinical signs or clinical symptoms or disease caused by an infection with PCV3 in an animal or for use in a method of treating or preventing an infection with PCV3 in an animal; wherein said animal is swine.

Also, the protein of the further consideration or the composition of the further consideration is provided for use in a method of reducing or preventing the clinical signs or clinical symptoms or disease caused by an infection with PCV3 in an animal or for use in a method of treating or preventing an infection with PCV3 in an animal; wherein said animal is a pig.

Also, the protein of the further consideration or the composition of the further consideration is provided for use in a method of reducing or preventing the clinical signs or clinical symptoms or disease caused by an infection with PCV3 in an animal or for use in a method of treating or preventing an infection with PCV3 in an animal; wherein said animal is a piglet.

Also, the protein of the further consideration or the composition of the further consideration is provided for use in a method of reducing or preventing the clinical signs or clinical symptoms or disease caused by an infection with PCV3 in an animal or for use in a method of treating or preventing an infection with PCV3 in an animal; wherein said animal is a piglet; and wherein the piglet is to be suckled by a sow to which the protein of the further consideration or the composition of the further consideration has been administered.

Also, the protein of the further consideration or the composition of the further consideration is provided for use in a method of reducing or preventing the clinical signs or disease caused by an infection with PCV3 in an animal or for use in a method of treating or preventing an infection with PCV3 in an animal; wherein said animal is a sow.

Also, the protein of the further consideration or the composition of the further consideration is provided for use in a method of reducing or preventing the clinical signs or clinical symptoms or disease caused by an infection with PCV3 in an animal or for use in a method of treating or preventing an infection with PCV3 in an animal; wherein said animal is a pregnant sow, gilt or pre-breeding gilt.

Also, the protein of the further consideration or the composition of the further consideration is provided for use in immunizing an animal against PCV3.

Also, the protein of the further consideration or the composition of the further consideration is provided for use in immunizing an animal against PCV3; wherein said animal is swine.

Also, the protein of the further consideration or the composition of the further consideration is provided for use in immunizing an animal against PCV3; wherein said animal is a pig.

Also, the protein of the further consideration or the composition of the further consideration is provided for use in immunizing an animal against PCV3; wherein said animal is a piglet.

Also, the protein of the further consideration or the composition of the further consideration is provided for use in immunizing an animal against PCV3; wherein said animal is a piglet; and wherein the piglet is to be suckled by a sow to which the protein of the further consideration or the composition of the further consideration has been administered.

Also, the protein of the further consideration or the composition of the further consideration is provided for use in immunizing an animal against PCV3; wherein said animal is a sow.

Also, the protein of the further consideration or the composition of the further consideration is provided for use in immunizing an animal against PCV3; wherein said animal is a pregnant sow, gilt or pre-breeding gilt.

Also, the protein of the further consideration or the composition of the further consideration is provided for use in a method of reducing or eliminating or abrogating PCV3 viral expression in an animal.

Also, the protein of the further consideration or the composition of the further consideration is provided for use in a method of reducing or eliminating or abrogating PCV3 viral expression in an animal; wherein said animal is swine.

Also, the protein of the further consideration or the composition of the further consideration is provided for use in a method of reducing or eliminating or abrogating PCV3 viral expression in an animal; wherein said animal is a pig.

Also, the protein of the further consideration or the composition of the further consideration is provided for use in a method of reducing or eliminating or abrogating PCV3 viral expression in an animal; wherein said animal is a piglet.

Also, the protein of the further consideration or the composition of the further consideration is provided for use in a method of reducing or eliminating or abrogating PCV3 viral expression in an animal; wherein said animal is a piglet; and wherein the piglet is to be suckled by a sow to which the protein of the further consideration or the composition of the further consideration has been administered.

Also, the protein of the further consideration or the composition of the further consideration is provided for use in a method of reducing or eliminating or abrogating PCV3 viral expression in an animal; wherein said animal is a sow.

Also, the protein of the further consideration or the composition of the further consideration is provided for use in a method of reducing or eliminating or abrogating PCV3 viral expression in an animal; wherein said animal is a pregnant sow, gilt or pre-breeding gilt.

Also, the protein of the further consideration or the composition of the further consideration is provided for use in inducing the production of antibodies specific for PCV3 in an animal.

Also, the protein of the further consideration or the composition of the further consideration is provided for use in inducing the production of antibodies specific for PCV3 in an animal; wherein said animal is swine.

Also, the protein of the further consideration or the composition of the further consideration is provided for use in inducing the production of antibodies specific for PCV3 in an animal; wherein said animal is a pig.

Also, the protein of the further consideration or the composition of the further consideration is provided for use in inducing the production of antibodies specific for PCV3 in an animal; wherein said animal is a piglet.

Also, the protein of the further consideration or the composition of the further consideration is provided for use in inducing the production of antibodies specific for PCV3 in an animal; wherein said animal is a piglet; and wherein the piglet is to be suckled by a sow to which the protein of the further consideration or the composition of the further consideration has been administered.

Also, the protein of the further consideration or the composition of the further consideration is provided for use in inducing the production of antibodies specific for PCV3 in an animal; wherein said animal is a sow.

Also, the protein of the further consideration or the composition of the further consideration is provided for use in inducing the production of antibodies specific for PCV3 in an animal; wherein said animal is a pregnant sow, gilt or pre-breeding gilt.

Preferably, said protein of the further consideration or the composition of the further consideration is administered intramuscularly or intradermally to said animal.

Preferably, said protein of the further consideration or the composition of the further consideration is administered to said animal in conjunction with another antigen, preferably wherein the other pathogen is an antigen to a porcine pathogen.

Preferably, said protein of the further consideration or the composition of the further consideration is administered to said animal in conjunction with another antigen; wherein said other antigen is not a PCV3 ORF2 antigen, preferably wherein the other pathogen is an antigen to a porcine pathogen.

Preferably, said protein of the further consideration or the composition of the further consideration is administered to said animal in conjunction with another antigen; wherein said other antigen is not a PCV3 antigen, preferably wherein the other pathogen is an antigen to a porcine pathogen.

Also, the protein of the further consideration or the composition of the further consideration is provided for any of the above uses, wherein said animal is a sow pregnant with a piglet.

Also, the protein of the further consideration or the composition of the further consideration is provided for any of the above uses, wherein said animal is a sow pregnant with a piglet; and wherein the piglet is to be suckled by a sow to which the protein of the further consideration or the composition according to a further consideration has been administered.

Also, the protein of the further consideration or the composition of the further consideration is provided for any of the above uses, wherein said animal is a sow; and wherein said protein of the further consideration or said composition of the further consideration is administered twice to said sow.

Preferably said animal is a sow; and wherein said protein of the further consideration or said composition of the further consideration is only administered twice to said sow.

Also, the protein of the further consideration or the composition of the further consideration is provided for any of the above uses wherein said animal is a piglet; and wherein the protein of the further consideration or the composition of the further consideration is administered once to said piglet.

Also, the protein of the further consideration or the composition of the further consideration is provided for any of the above uses wherein said animal is a piglet; and wherein the protein of the further consideration or a composition of the further consideration is only administered once to said piglet.

Also, the protein of the further consideration or the composition of the further consideration is provided for any of the above uses wherein said animal is a sow; and wherein the protein of the further consideration or a composition of the further consideration is administered twice to said sow; and wherein said use does not include the administration of any other PCV3 antigen to said animal before or during the administration of said protein of the further consideration or composition of the further consideration.

Also, the protein of the further consideration or the composition of the further consideration is provided for any of the above uses wherein said animal is a sow; and wherein the protein of the further consideration or the composition of the further consideration is administered twice to said sow; and wherein said use does not include the administration of any other PCV3 antigen to said animal before or during the administration of said protein of the further consideration or the composition of the further consideration.

Also, the protein of the further consideration or the composition of the further consideration is provided for any of the above uses wherein said animal is a sow; and wherein the protein of the further consideration or the composition of the further consideration is administered only twice to said sow; and wherein said use does not include the administration of any other PCV3 antigen to said animal before or during the administration of said protein of the further consideration or the composition of the further consideration.

Also, the protein of the further consideration or the composition of the further consideration is provided for any of the above uses wherein said animal is a piglet; and wherein the protein of the further consideration or the composition of the further consideration is administered once to said piglet; and wherein said use does not include the administration of any other PCV3 antigen to said animal before or during the administration of said protein of the further consideration or the composition of the further consideration.

Also, the protein of the further consideration or the composition of the further consideration is provided for any of the above uses wherein said animal is a piglet; and wherein the protein of the further consideration or the composition of the further consideration is administered once to said piglet; and wherein said use does not include the administration of any other PCV3 antigen to said animal before or during the administration of said protein of the further consideration or the composition of the further consideration.

Also, the protein of the further consideration or the composition of the further consideration is provided for any of the above uses wherein said animal is a piglet; is administered only once to said piglet; and wherein said use does not include the administration of any other PCV3 antigen to said animal before or during the administration of said protein of the further consideration or the composition of the further consideration.

Also, the protein of the further consideration or the composition of the further consideration is provided for any of the above uses wherein the administration to the animal in the use consists of a single, one shot administration or a single, one dose administration of said protein of the further consideration or the composition of the further consideration.

Also, the protein of the further consideration or the composition of the further consideration is provided for any of the above uses wherein the administration to the animal in the use consists of a multi-shot or multi-dose regimen of said protein of the further consideration or the composition of the further consideration.

Also, the protein of the further consideration or the composition of the further consideration is provided for any of the above uses wherein the administration to the animal in the use consists of a double shot administration; or a dual dose administration of said protein of the further consideration or the composition of the further consideration.

Also, the protein of the further consideration or the composition of the further consideration is provided for any of the above uses wherein the administration to the animal occurs within at least 1 or 2 or 3 weeks of exposure to virulent Porcine Circovirus.

Also, the protein of the further consideration or the composition of the further consideration is provided wherein the animal is a piglet not older than 15 weeks of age, or not older than 6 weeks of age, or not older than 3 weeks of age, or not older than 2 weeks of age, or not older than 1 week of age.

Also, the protein of the further consideration or the composition of the further consideration is provided for any of the above uses wherein said protein of the further consideration is for any of the above uses.

Also, the protein of the further consideration or the composition of the further consideration is provided for any of the above uses wherein said protein of the further consideration is for the use of two or more uses mentioned above.

Also, the protein of the further consideration or the composition of the further consideration is provided for any of the above uses wherein said composition of the further consideration is for any of the above uses.

Also, the protein of the further consideration or the composition of the further consideration is provided for any of the above uses wherein a second antigen is administered to the animal before administration of the protein of the further consideration or the composition of the further consideration.

Also, the protein of the further consideration or the composition of the further consideration is provided for any of the above uses wherein a second antigen is administered to the animal at the same time as administration of the protein of the further consideration or a composition of the further consideration.

Also, the protein of the further consideration or the composition of the further consideration is provided for any of the above uses wherein a second antigen is administered to the animal at the same time and in the same composition as administration of the protein of the further consideration or the composition of the further consideration.

Also, the protein of the further consideration or the composition of the further consideration is provided for any of the above uses wherein a second antigen is administered to the animal at the same time and in a different composition as administration of the protein of the further consideration or the composition of the further consideration.

Also, the protein of the further consideration or the composition of the further consideration is provided for any of the above uses wherein a second antigen is administered to the animal after the administration of the protein of the further consideration or a composition of the further consideration.

Also, the protein of the further consideration or the composition of the further consideration is provided for use in the vaccination of a pig to lessen the severity of clinical signs or clinical symptoms resulting from PCV3 infection in the pig, wherein the protein is in an immunogenic composition that is administered in one dose to the pig.

Also, the protein of the further consideration or the composition of the further consideration is provided for use in the vaccination of a pig to lessen the severity of clinical signs or clinical symptoms resulting from PCV3 infection in the pig, wherein the protein is in an immunogenic composition that is administered in only one dose to the pig.

Also, the protein of the further consideration or the composition of the further consideration is provided for use in the vaccination of a pig to lessen the severity of clinical signs or clinical symptoms resulting from PCV3 infection in the pig, wherein the protein is in an immunogenic composition that is administered in two doses to the pig.

Also, the protein of the further consideration or the composition of the further consideration is provided for use in the vaccination of a pig to lessen the severity of clinical signs or clinical symptoms resulting from PCV3 infection in the pig, wherein the protein is in an immunogenic composition that is administered in only two doses to the pig.

Also, the protein of the further consideration or the composition of the further consideration is provided for use as the single PCV3 antigen for vaccination of a pig to lessen the severity of clinical signs or clinical symptoms resulting from PCV3 infection in the pig, wherein the protein is in an immunogenic composition that is administered in one dose to the pig.

Also, the protein of the further consideration or the composition of the further consideration is provided for use as the single PCV3 antigen for vaccination of a pig to lessen the severity of clinical signs or clinical symptoms resulting from PCV3 infection in the pig, wherein the protein is in an immunogenic composition that is administered in only one dose to the pig.

Also, the protein of the further consideration or the composition of the further consideration is provided for use as the single PCV3 antigen for vaccination of a pig to lessen the severity of clinical signs or clinical symptoms resulting from PCV3 infection in the pig, wherein the protein is in an immunogenic composition that is administered in two doses to the pig.

Also, the protein of the further consideration or the composition of the further consideration is provided for use as the single PCV3 antigen for vaccination of a pig to lessen the severity of clinical signs or clinical symptoms resulting from PCV3 infection in the pig, wherein the protein is in an immunogenic composition that is administered in only two doses to the pig.

Also, the protein of the further consideration or the composition of the further consideration is provided for lessening the severity of clinical signs or clinical symptoms resulting from PCV3 infection in a pig;
wherein one dose of the immunogenic composition is administered to the pig in a vaccination method to lessen the severity of clinical signs or clinical symptoms resulting from PCV3 infection in the pig;
wherein the administration of the one dose of the immunogenic composition to the pig in the vaccination method lessens the severity of clinical signs or clinical symptoms resulting from PCV3 infection in the pig;
wherein the protein of the further consideration is the antigenic component in the one dose of the immunogenic composition in the vaccination method that lessens the severity of clinical signs or clinical symptoms resulting from PCV3 infection in the pig;
preferably wherein the protein is in an amount of at least 2 μg in the one dose of the immunogenic composition;
wherein the protein is the antigenic component in the vaccination method that lessens the severity of clinical signs or clinical symptoms resulting from PCV3 infection in the pig.

Also provided herein is an immunogenic composition of the further consideration for lessening the severity of clinical signs or clinical symptoms resulting from PCV3 infection in a pig;
wherein only one dose of the immunogenic composition is administered to the pig in a vaccination method to lessen the severity of clinical signs or clinical symptoms resulting from PCV3 infection in the pig;
wherein the administration of the one dose of the immunogenic composition to the pig in the vaccination method lessens the severity of clinical signs or clinical symptoms resulting from PCV3 infection in the pig;
wherein the protein of the further consideration is the antigenic component in the one dose of the immunogenic composition in the vaccination method that lessens the severity of clinical signs or clinical symptoms resulting from PCV3 infection in the pig;
preferably wherein the protein is in an amount of at least 2 μg in the one dose of the immunogenic composition;
wherein the protein is the antigenic component in the vaccination method that lessens the severity of clinical signs or clinical symptoms resulting from PCV3 infection in the pig.

Also, the protein of the further consideration or the composition of the further consideration is provided for lessening the severity of clinical signs or clinical symptoms resulting from PCV3 infection in a pig;
wherein two doses of the immunogenic composition is administered to the pig in a vaccination method to lessen the severity of clinical signs or clinical symptoms resulting from PCV3 infection in the pig;
wherein the administration of the two doses of the immunogenic composition to the pig in the vaccination method lessens the severity of clinical signs or clinical symptoms resulting from PCV3 infection in the pig;
wherein the protein of the further consideration is the antigenic component in the two doses of the immunogenic composition in the vaccination method that lessens the severity of clinical signs or clinical symptoms resulting from PCV3 infection in the pig;
preferably wherein the protein is in an amount of at least 2 μg in the one dose of the immunogenic composition;
wherein the protein is the antigenic component in the vaccination method that lessens the severity of clinical signs or clinical symptoms resulting from PCV3 infection in the pig.

Also, an immunogenic composition of the further consideration is provided for lessening the severity of clinical signs or clinical symptoms resulting from PCV3 infection in a pig;
wherein only two doses of the immunogenic composition is administered to the pig in a vaccination method to lessen the severity of clinical signs or clinical symptoms resulting from PCV3 infection in the pig;
wherein the administration of the two doses of the immunogenic composition to the pig in the vaccination method lessens the severity of clinical signs or clinical symptoms resulting from PCV3 infection in the pig;
wherein the protein of the further consideration is the antigenic component in the two doses of the immunogenic composition in the vaccination method that lessens the severity of clinical signs or clinical symptoms resulting from PCV3 infection in the pig;
preferably wherein the protein is in an amount of at least 2 μg in the one dose of the immunogenic composition;
wherein the protein is the antigenic component in the vaccination method that lessens the severity of clinical signs or clinical symptoms resulting from PCV3 infection in the pig.

Preferably, in any of the above-mentioned uses, said clinical signs or symptoms are selected from the group consisting of reduction of average daily weight gain and mortality.

Preferably, in any of the above-mentioned uses, said clinical signs or symptoms are selected from the group consisting of gross lesions, histological lesions, replication of PCV3 in a tissue, and PCV3 viremia.

Preferably, in any of the above-mentioned uses, said clinical signs or symptoms are selected from the group consisting of development or production of a mummified, stillborn and/or weak fetus.

Preferably, in any of the above-mentioned uses, said clinical signs or symptoms is or include expelling of a mummified, stillborn and/or weak fetus.

The present invention will now be described by way of the following sets of clauses. For ease of reference, these sets of clauses have been labelled Clause Set A, Clause Set B etc. The disclosure in each set of clauses is equally applicable to the present invention. Likewise the disclosure in each set of clauses is equally applicable to every other set of clauses:

### Clause Set a:

Clause Set A—The present invention will now be described by way of the following set of numbered clauses (Clause Set A). The disclosure in this set of clauses is equally applicable to the present invention. Likewise the disclosure in this set of clauses is equally applicable to each of the other set of clauses.

1. A composition comprising:
porcine circovirus type 3 (PCV3) ORF2 protein; and
a veterinary-acceptable carrier comprising a solvent, a dispersion media, a coating, a stabilizing agent, a diluent, a preservative, an anti-microbial agent, an antifungal agent, an isotonic agent, an adsorption delaying agent, an adjuvant, cell culture supernatant, a stabilizing agent, a viral or expression vector, and an immunomodulatory agent or any combination thereof.

2. The composition of clause 1, wherein the veterinary-acceptable carrier comprises an adjuvant, immunomodulatory agent, cell culture supernatant, viral or expression vector or any combination thereof.

3. The composition of clause 1, wherein the veterinary-acceptable carrier comprises an adjuvant.

4. The composition of any of clauses 1-3, wherein the PCV3 ORF2 is from group a1, b1 or b2 (using the subtyping designation of Fux et al., “Full genome characterization of porcine circovirus type 3 isolates reveals the existence of two distinct groups of virus strains,” Virology Journal (2018) 15:25, DOI 10.1186/s12985-018-0929-3 (incorporated herein by reference); see, e.g., Table 4).

5. The composition of any of clauses 1-3, wherein the PCV3 ORF2 protein comprises or consists of an amino acid sequence encoded by a polynucleotide sequence having at least 90%, or at least 91%, or at least 92%, or at least 93%, or at least 94%, or at least 95%, or at least 96%, or at least 97%, or at least 98%, or at least 99%, or 100% sequence identity with SEQ ID NO:1 sequence identity or sequence homology with SEQ ID NO:1.

6. The composition of any of clauses 1-5, wherein the PCV3 ORF2 protein is a recombinant PCV3 ORF2 protein from expression thereof by an expression vector, comprising a polynucleotide sequence that encodes the PCV3 ORF2 protein.

7. The composition of clause 6, wherein the expression vector is a baculovirus.

8. The composition of any one of clauses 1-7, further comprising a PCV2 ORF2 protein.

9. The composition of clause 8, wherein the PCV2 ORF2 protein is from expression by an expression vector, comprising a polynucleotide sequence that encodes the PCV2 ORF2 protein.

10. The composition of clause 9, wherein the expression vector is a baculovirus.

11. The composition of any one of clauses 1-10, further comprising an additional antigen of an additional porcine pathogen.

12. The composition of clause 11, wherein the additional antigen of an additional porcine pathogen comprises a PRRSV (porcine respiratory and reproductive syndrome virus) antigen, a Mycoplasma hyopneumoniae bacterin antigen, a Mycoplasma hyopneumoniae supernatant antigen, an Aujeszky's disease or pseudorabies antigen, a swine influenza antigen, a swine fever antigen (classical or African or combination thereof), an Actinobacillus pleuropneumoniac antigen, an Escherichia coli antigen, or a Pasteurella multocida antigen.

13. The composition of any of clauses 1-12, wherein the PCV3 ORF2 protein is present in an amount of 0.2 to about 400 μg/ml, or about 0.3 to about 200 μg/ml, or about 0.35 to about 100 μg/ml, or about 0.4 to about 50 μg/ml, or about 0.45 to about 30 g/ml, or about 0.6 to about 15 μg/ml, or about 0.75 to about 8 μg/ml, or about 1.0 to about 6 μg/ml, or about 1.3 to about 3.0 μg/ml, or about 1.4 to about 2.5 μg/ml, or about 1.5 to about 2.0 μg/ml, or about 1.6 μg/ml.

14. The composition of any of clauses 1-12, wherein the PCV3 ORF2 protein or total PCV2 and PCV3 ORF2 proteins are present in an amount of about 0.2 to about 400 μg/dose, or about 0.3 to about 200 μg/dose, or about 0.35 to about 100 μg/dose, or about 0.4 to about 50 μg/dose, or about 0.45 to about 30 μg/dose, or about 0.6 to about 15 μg/dose, or about 0.75 to about 8 μg/dose, or about 1.0 to about 6 μg/dose, or about 1.3 to about 3.0 μg/dose, or about 1.4 to about 2.5 μg/dose, or about 1.5 to about 2.0 μg/dose, or about 1.6 μg/dose.

15. The composition of any one of clauses 1-14, wherein the adjuvant comprises aluminum hydroxide; aluminum phosphate; a saponin; Quil A; QS-21; GPI-0100; a water-in-oil emulsion; an oil-in-water emulsion; a water-in-oil-in-water emulsion; an emulsion based on light liquid paraffin oil or European Pharmacopea type adjuvant; an isoprenoid oil; squalane; squalene oil resulting from oligomerization of alkenes or isobutene or decene; (an) ester(s) of acid(s) or of alcohol(s) containing a linear alkyl group; plant oil(s); ethyl oleate; propylene glycol di-(caprylate/caprate); glyceryl tri-(caprylate/caprate); propylene glycol dioleate; (an) ester(s) of branched fatty acid(s) or alcohol(s); isostearic acid ester(s); nonionic surfactant(s); (an) ester(s) of sorbitan or of mannide or of glycol or of polyglycerol or of propylene glycol or of oleic, or isostearic acid or of ricinoleic acid or of hydroxystearic acid, optionally ethoxylated, anhydromannitol oleate; polyoxypropylene-polyoxyethylene copolymer blocks, a Pluronic product, a carbopol; Carbopol 974P; Carbopol 934P; Carbopol 971P; a polymer of acrylic or methacrylic acid; copolymer of maleic anhydride and alkenyl derivative; a polymer of acrylic or methacrylic acid which is cross-linked; a polymer of acrylic or methacrylic acid which is cross-linked with a polyalkenyl ether of sugar or polyalcohol; a carbomer; an acrylic polymer cross-linked with a polyhydroxylated compound having at least 3 and not more than 8 hydroxyl groups with hydrogen atoms of at least three hydroxyls optionally or being replaced by unsaturated aliphatic radicals having at least 2 carbon atoms with said radicals containing from 2 to 4 carbon atoms such as vinyls, allyls and other ethylenically unsaturated groups and the unsaturated radicals may themselves contain other substituents, such as methyl; RIBI adjuvant system; Block co-polymer; SAF-M; monophosphoryl lipid A; Avridine lipid-amine adjuvant; heat-labile enterotoxin from E. coli (recombinant or otherwise); cholera toxin; IMS 1314, or muramyl dipeptide.

16. The composition of any one of clauses 1-15, comprising from about 50 μg to about 2000 μg of adjuvant; or wherein adjuvant present in an amount about 250 μg/ml dose of the composition, or wherein the adjuvant is present in an amount of about 100 μg to about 10 mg per dose; or wherein the adjuvant is present in an amount of about 500 μg to about 5 mg per dose; the adjuvant is present in an amount of about 750 μg to about 2.5 mg per dose; or the adjuvant is present in an amount of about 1 mg per dose.

17. The composition of any one of clauses 1-16, wherein immunomodulatory agent comprises interleukin(s), interferon(s), or other cytokine(s), or keyhole limpet hemocyanin (KLH), or KLH emulsified with incomplete Freund's adjuvant (KLH/ICFA).

18. The composition of any one of clauses 1-17, wherein comprising from about 1 μg/ml to about 60 μg/ml of antibiotic(s), or less than about 30 μg/ml of antibiotic(s).

19. The composition of any one of clauses 1-18, wherein the antibiotic(s) comprise Gentamicin.

20. The composition of any one of clauses 1-19, comprising (i) PCV3 ORF2 protein, (ii) at least a portion of baculovirus that expressed said PCV3 ORF2 protein, (iii) a portion of cell culture of cells that were infected or transfected with recombinant baculovirus that expressed said PCV3 ORF2 protein, (iv) inactivating agent or inactivating agent comprising binary ethyleneimine (BEI), (v) sodium thiosulfate or sodium thiosulfate in equivalent amounts to inactivating agent or BEI; (vi) adjuvant or adjuvant comprising Carbopol or Carbopol 971, and
(vii) phosphate salt in a physiologically acceptable concentration.

21. The composition of clause 20, wherein about 90% of the components (i) to (iii) have a size smaller than 1 μm and the pH of said composition is adjusted to about 6.5 to 7.5

22. The composition of clauses 20 or 21 wherein the BEI is from the cell culture having been treated with about 2 to 8 or about 5 mM BEI to inactivate the baculovirus and/or the composition contains about 2 to 8 or about 5 mM BEI and/or the composition contains about 1 mg of the Carbopol or Carbopol 971.

23. The composition of any one of clauses 1-22, formulated and/or packaged for a single dose or one shot administration, and not a multi-dose regimen.

24. A method for eliciting an immune response or an immunological response or a protective immune or immunological response against (i) PCV3 and/or (ii) PCV2 and PCV3 and/or (iii) PCV3 and another porcine pathogen and/or (iv) PCV3, PCV2 and another porcine pathogen, comprising administering to an animal a composition as defined in any of clauses 1-23.

25. The method of clause 25 wherein the animal is a porcine.

26. The method of clause 25, wherein the porcine is a pig or piglet.

27. The method of clause 26, wherein the pig or piglet is not older than 15 weeks of age, or not older than 6 weeks of age, or not older than 3 weeks of age, or not older than 2 weeks of age, or not older than 1 week of age.

28. The method of clause 26, wherein the administration occurs within at least 1 or 2 or 3 weeks of exposure to virulent Porcine Circovirus.

29. The method of any one of clauses 24-28, wherein the administration comprises a single, one shot administration; or a single, one dose administration; and not a multi-shot or multi-dose regimen.

30. Use of a composition of any one of clauses 1-23 in a method of any one of clauses 24-29; or use of a PCV3 ORF2 protein, alone or in combination, of any one of the compositions of clauses 1-23, for use in the preparation of a composition for inducing an immunological or immune response or a protective immune or immunological response against (i) PCV3 and/or (ii) PCV2 and PCV3 and/or (iii) PCV3 and another porcine pathogen and/or (iv) PCV3, PCV2 and another porcine pathogen, or for use in a method for inducing an immunological or immune response or a protective immune or immunological response against (i) PCV3 and/or (ii) PCV2 and PCV3 and/or (iii) PCV3 and another porcine pathogen and/or (iv) PCV3, PCV2 and another porcine pathogen.

31. A method for preparing a composition as defined in any one of clauses 1-23, comprising producing the PCV3 ORF2 protein by a baculovirus expression system in cultured insect cells.

32. The method of clause 31, including inactivating the baculovirus.

33. The method of clause 32, wherein the inactivating comprises heat treatment or use of a virus inactivating agent.

34. The method of clause 25, wherein the virus inactivating agent comprises an aziridine compound.

35. The method of clause 26, wherein the aziridine compound comprises BEI.

36. A recombinant vector comprising a polynucleotide sequence that encodes a polypeptide sequence that encodes a PCV3 ORF2 protein.

37. The recombinant vector of clause 36, wherein the PCV3 ORF2 is from group a1, b1 or b2 (using the subtyping designation of Fux et al., “Full genome characterization of porcine circovirus type 3 isolates reveals the existence of two distinct groups of virus strains,” Virology Journal (2018) 15:25, DOI 10.1186/s12985-018-0929-3 (incorporated herein by reference); see, e.g., Table 4).

38. The recombinant vector of clause 36, wherein the PCV3 ORF2 protein comprises or consists of an amino acid sequence encoded by a polynucleotide sequence having at least 90%, or at least 91%, or at least 92%, or at least 93%, or at least 94%, or at least 95%, or at least 96%, or at least 97%, or at least 98%, or at least 99%, or 100% sequence identity or sequence homology with SEQ ID NO:1.

39. The recombinant vector of any of clauses 36-38, wherein the recombinant vector is a baculovirus.

40. The recombinant vector of clause 39, wherein the recombinant vector comprises at least 90% or at least 91%, or at least 92%, or at least 93%, or at least 94%, or at least 95%, or at least 96%, or at least 97%, or at least 98%, or at least 99%, or 100% sequence identity or sequence homology with SEQ ID NO:2.

### Clause Set B:

Clause Set B—The present invention will now be described by way of the following set of numbered clauses (Clause Set B). The disclosure in this set of clauses is equally applicable to the present invention. Likewise the disclosure in this set of clauses is equally applicable to each of the other set of clauses.

1. A composition comprising a porcine circovirus type 3 (PCV3) ORF2 protein, preferably an antigenic PCV3 ORF2 protein (a PCV3 ORF2 antigen).

2. The composition of clause 1, further comprising a veterinary-acceptable carrier selected from the group consisting of a solvent, a dispersion media, a coating, a stabilizing agent, a diluent, a preservative, an anti-microbial agent, an antifungal agent, an isotonic agent, an adsorption delaying agent, an adjuvant, cell culture supernatant, a stabilizing agent, a viral vector, an expression vector, an immunomodulatory agent, and/or any combination thereof.

3. A composition, in particular the composition of clause 1 or 2, comprising: porcine circovirus type 3 (PCV3) ORF2 protein; and a veterinary-acceptable carrier comprising a solvent, a dispersion media, a coating, a stabilizing agent, a diluent, a preservative, an anti-microbial agent, an antifungal agent, an isotonic agent, an adsorption delaying agent, an adjuvant, cell culture supernatant, a stabilizing agent, a viral or expression vector, an immunomodulatory agent and/or any combination thereof.

4. The composition of any one of clauses 1 to 3, wherein the veterinary-acceptable carrier comprises an adjuvant, immunomodulatory agent, cell culture supernatant, viral or expression vector or any combination thereof.

5. The composition of any one of clauses 1 to 4, wherein the veterinary-acceptable carrier comprises an adjuvant.

6. The composition of any of clauses 1 to 5, wherein the PCV3 is selected from the group consisting of PCV3a and PCV3b.

7. The composition of any of clauses 1 to 6, wherein the PCV3 is any phylogenetic clade of PCV3 or selected from the group consisting PCV3a1, PCV3b1, PCV3b2 and PCV3c.

8. The composition of any of clauses 1 to 7, wherein the PCV3 ORF2 is from group a1, b1 or b2.

9. The composition of any of clauses 1 to 8, wherein the PCV3 ORF2 protein comprises or consists of an amino acid sequence encoded by a polynucleotide sequence having at least 90%, or at least 91%, or at least 92%, or at least 93%, or at least 94%, or at least 95%, or at least 96%, or at least 97%, or at least 98%, or at least 99%, or 100% sequence identity with SEQ ID NO:1 or sequence homology with SEQ ID NO:1.

10. The composition of any of clauses 1-9, wherein the PCV3 ORF2 protein comprises or consists of an amino acid sequence having at least 90% sequence identity with the sequence of SEQ ID NO: 4.

11. The composition of any of clauses 1 to 10, wherein the PCV3 ORF2 protein is a recombinant PCV3 ORF2 protein.

12. The composition of any of clauses 1 to 11, wherein the PCV3 ORF2 protein is a recombinant PCV3 ORF2 protein from expression thereof by an expression vector, comprising a polynucleotide sequence that encodes the PCV3 ORF2 protein.

13. The composition of clause 12, wherein the expression vector is a baculovirus.

14. The composition of any of clauses 1 to 13, wherein the PCV3 ORF2 protein is a recombinant baculovirus expressed PCV3 ORF2.

15. The composition of any one of clauses 1 to 14, further comprising a PCV2 ORF2 protein, preferably an antigenic PCV2 ORF2 protein (a PCV2 ORF2 antigen).

16. The composition of clause 15, wherein the PCV2 ORF2 protein is from expression by an expression vector, comprising a polynucleotide sequence that encodes the PCV2 ORF2 protein.

17. The composition of clause 16, wherein the expression vector is a baculovirus.

18. The composition of any one of clauses 1 to 17, further comprising an additional antigen of an additional porcine pathogen.

19. The composition of clause 18, wherein the additional antigen of an additional porcine pathogen comprises a PRRSV (porcine respiratory and reproductive syndrome virus) antigen, a Mycoplasma hyopneumoniae bacterin antigen, a Mycoplasma hyopneumoniae supernatant antigen, an Aujeszky's disease or pseudorabies antigen, a swine influenza antigen, a swine fever antigen (classical or African or combination thereof), an Actinobacillus pleuropneumoniae antigen, an Escherichia coli antigen, a porcine parvovirus (PPV) antigen or a Pasteurella multocida antigen, or a combination thereof.

20. The composition of any of clauses 1 to 19, wherein the PCV3 ORF2 protein is present in an amount of 0.2 to about 400 μg/ml, or about 0.3 to about 200 μg/ml, or about 0.35 to about 100 μg/ml, or about 0.4 to about 50 μg/ml, or about 0.45 to about 30 μg/ml, or about 0.6 to about 15 μg/ml, or about 0.75 to about 8 μg/ml, or about 1.0 to about 6 μg/ml, or about 1.3 to about 3.0 μg/ml, or about 1.4 to about 2.5 μg/ml, or about 1.5 to about 2.0 μg/ml, or about 1.6 μg/ml.

21. The composition of any of clauses 1 to 20, wherein the PCV3 ORF2 protein or total PCV2 and PCV3 ORF2 proteins are present in an amount of about 0.2 to about 400 μg/dose, or about 0.3 to about 200 μg/dose, or about 0.35 to about 100 μg/dose, or about 0.4 to about 50 μg/dose, or about 0.45 to about 30 μg/dose, or about 0.6 to about 15 μg/dose, or about 0.75 to about 8 μg/dose, or about 1.0 to about 6 μg/dose, or about 1.3 to about 3.0 μg/dose, or about 1.4 to about 2.5 μg/dose, or about 1.5 to about 2.0 μg/dose, or about 1.6 μg/dose.

22. The composition of any one of clauses 2 to 21, wherein the adjuvant comprises a polymer of acrylic or methacrylic acid; copolymer of maleic anhydride and alkenyl derivative; a polymer of acrylic or methacrylic acid which is cross-linked; a polymer of acrylic or methacrylic acid which is cross-linked with a polyalkenyl ether of sugar or polyalcohol; a carbomer; an acrylic polymer cross-linked with a polyhydroxylated compound having at least 3 and not more than 8 hydroxyl groups with hydrogen atoms of at least three hydroxyls optionally or being replaced by unsaturated aliphatic radicals having at least 2 carbon atoms with said radicals containing from 2 to 4 carbon atoms such as vinyls, allyls and other ethylenically unsaturated groups and the unsaturated radicals may themselves contain other substituents, such as methyl; a carbopol; Carbopol 974P; Carbopol 934P; Carbopol 971P; aluminum hydroxide; aluminum phosphate; a saponin; Quil A; QS-21; GPI-0100; a water-in-oil emulsion; an oil-in-water emulsion; a water-in-oil-in-water emulsion; an emulsion based on light liquid paraffin oil or European Pharmacopea type adjuvant; an isoprenoid oil; squalane; squalene oil resulting from oligomerization of alkenes or isobutene or decene; (an) ester(s) of acid(s) or of alcohol(s) containing a linear alkyl group; plant oil(s); ethyl oleate; propylene glycol di-(caprylate/caprate); glyceryl tri-(caprylate/caprate); propylene glycol dioleate; (an) ester(s) of branched fatty acid(s) or alcohol(s); isostearic acid ester(s); nonionic surfactant(s); (an) ester(s) of sorbitan or of mannide or of glycol or of polyglycerol or of propylene glycol or of oleic, or isostearic acid or of ricinoleic acid or of hydroxystearic acid, optionally ethoxylated, anhydromannitol oleate; polyoxypropylene-polyoxyethylene copolymer blocks, a Pluronic product, RIBI adjuvant system; Block co-polymer; SAF-M; monophosphoryl lipid A; Avridine lipid-amine adjuvant; heat-labile enterotoxin from E. coli (recombinant or otherwise); cholera toxin; IMS 1314, or muramyl dipeptide.

23. The composition of any one of clauses 2 to 22, comprising from about 50 μg to about 2000 μg of adjuvant; or wherein adjuvant present in an amount about 250 μg/ml dose of the composition, or wherein the adjuvant is present in an amount of about 100 μg to about 10 mg per dose; or wherein the adjuvant is present in an amount of about 500 μg to about 5 mg per dose; the adjuvant is present in an amount of about 750 μg to about 2.5 mg per dose; or the adjuvant is present in an amount of about 1 mg per dose.

24. The composition of any one of clauses 2 to 23, wherein immunomodulatory agent comprises interleukin(s), interferon(s), or other cytokine(s).

25. The composition of any one of clauses 1 to 24, wherein said composition comprises from about 1 μg/ml to about 60 μg/ml of antibiotic(s), or less than about 30 μg/ml of antibiotic(s).

26. The composition of any one of clauses 1 to 25, wherein the antibiotic(s) comprise Gentamicin.

27. The composition of any one of clauses 1 to 26, comprising (i) PCV3 ORF2 protein, (ii) at least a portion of baculovirus that expressed said PCV3 ORF2 protein, (iii) a portion of cell culture of cells that were infected or transfected with recombinant baculovirus that expressed said PCV3 ORF2 protein, (iv) inactivating agent or inactivating agent comprising binary ethyleneimine (BEI), (v) sodium thiosulfate or sodium thiosulfate in equivalent amounts to inactivating agent or BEI; (vi) adjuvant or adjuvant comprising Carbopol or Carbopol 971, and
(vii) phosphate salt in a physiologically acceptable concentration.

28. The composition of clause 27, wherein about 90% of the components (i) to (iii) have a size smaller than 1 μm and the pH of said composition is adjusted to about 6.5 to 7.5.

29. The composition of clauses 27 or 28 wherein the BEI is from the cell culture having been treated with about 2 to 8 or about 5 mM BEI to inactivate the baculovirus and/or the composition contains about 2 to 8 or about 5 mM BEI and/or the composition contains about 1 mg of the Carbopol or Carbopol 971.

30. The composition of any one of clauses 1 to 29, wherein said composition is formulated and/or packaged for a single dose or one shot administration of the composition, and not a multi-dose regimen; or wherein said composition is formulated and/or packaged for a multi-dose regimen of the composition.

31. The composition of any one of clauses 1 to 30, wherein the composition is an immunogenic composition.

32. The composition of any one of clauses 1 to 31 for use as a medicament.

33. The composition of any one of clauses 1 to 31 for use as a vaccine.

34. The composition of any one of clauses 1 to 31 for use in method for eliciting an immune response or an immunological response or a protective immune or immunological response against (i) PCV3 and/or (ii) PCV2 and PCV3 and/or (iii) PCV3 and another porcine pathogen and/or (iv) PCV3, PCV2 and another porcine pathogen.

35. The composition of any one of clauses 1 to 31 for use in a method of reducing or preventing the clinical signs or disease caused by an infection with PCV3 in an animal or for use in a method of treating or preventing an infection with PCV3 in an animal, and wherein said animal is preferably a pig.

36. The composition of any one of clauses 1 to 31 for use in a method for inducing an immune response against PCV3 in a pig, in particular in a preferably pregnant sow.

37. The composition of any one of clauses 1 to 31 for use in a method of reducing or preventing the clinical signs or disease caused by an infection with a PCV3 in a piglet, wherein the piglet is to be suckled by a sow to which the composition has been administered.

38. The composition for use according to clause 37, wherein said sow to which the composition has been administered is a sow to which the immunogenic composition has been administered while said sow has been pregnant, in particular with said piglet, or a pre-breeding gilt.

39. The composition for use according to any one of clauses 32 to 38, wherein said composition is to be administered intramuscularly or intradermally.

40. The composition for use according to any one of clauses 36 to 39, wherein said composition is to be administered intramuscularly or intradermally to said sow.

41. A method for eliciting an immune response or an immunological response or a protective immune or immunological response against (i) PCV3 and/or (ii) PCV2 and PCV3 and/or (iii) PCV3 and another porcine pathogen and/or (iv) PCV3, PCV2 and another porcine pathogen, comprising administering to an animal a composition as claused in any of clauses 1 to 31.

42. The method of clause 41 wherein the animal is a porcine.

43. The method of clause 42, wherein the porcine is a pig or piglet.

44. The method of clause 42 or 43, wherein the porcine is a sow.

45. A method of immunizing a subject comprising administering to the subject a composition according to any one of clauses 1 to 31.

46. A method of immunizing swine against a clinical disease caused by at least one pathogen in said animal, said method comprising the step of administering to the animal the composition according to any one of clauses 1 to 31, wherein said immunogenic composition fails to cause clinical signs of infection but is capable of inducing an immune response that immunizes the animal against pathogenic forms of said at least one pathogen.

47. The method of clause 46, wherein said at least one pathogen is PCV3.

48. A method for inducing the production of antibodies specific for PCV3 in a sow, wherein said method comprises administering the composition according to any one of clauses 1 to 31 to said sow.

49. A method of reducing or preventing the clinical signs or clinical symptoms caused by an infection with a PCV3 in a piglet, wherein said method comprises administering the composition according to any one of clauses 1 to 31 to a sow, and allowing said piglet to be suckled by said sow.

50. The method of clause 49, wherein said sow is a sow being pregnant, in particular with said piglet, or a pre-breeding gilt.

51. The method of clause 49 or 50, comprising the steps of administering the composition according to any one of clauses 1 to 31 to a sow being pregnant with said piglet, allowing said sow to give birth to said piglet, and allowing said piglet to be suckled by said sow.

52. A method of reducing the clinical signs and/or clinical symptoms caused by an infection with a PEDV in a piglet, wherein the piglet is to be suckled by a sow to which the composition of any one of clauses 1 to 31 has been administered.

53. The method of any one of clauses 45 to 52, wherein said immunogenic composition or said vaccine or pharmaceutical composition is administered intramuscularly or intradermally to said sow.

54. The method of any one of clauses 45 to 53, wherein said immunogenic composition or said vaccine or pharmaceutical composition is administered twice to said sow.

55. The method of any one of clauses 45 to 54, wherein said immunogenic composition or said vaccine or pharmaceutical composition is administered twice mucosally, preferably twice intranasally, to said sow.

56. The composition for use according to any one of clauses 32-40 or the method of any one of clauses 41 to 55, wherein said clinical signs are selected from the group consisting of reduction of average daily weight gain and mortality.

57. The composition for use according to any one of clauses 32-40 or the method of any one of clauses 41 to 55, wherein the clinical signs are selected from the group consisting of expelling of a mummified, stillborn and/or weak fetus.

58. The composition for use according to any one of clauses 32 to 40 or the method of any one of clauses 41 to 55, wherein the clinical symptoms are selected from the group consisting of, gross lesions, histologic lesions, replication of PCV3 in a tissue, and PCV3 viremia.

59. The composition for use according to any one of clauses 32 to 40 or the method of any one of clauses 41 to 55, wherein the clinical symptoms are selected from the group consisting of development or production of a mummified, stillborn and/or weak fetus.

60. The composition for use according to any one of clauses 32 to 40 or the method of any one of clauses 41 to 55, wherein the pig or piglet is not older than 15 weeks of age, or not older than 6 weeks of age, or not older than 3 weeks of age, or not older than 2 weeks of age, or not older than 1 week of age.

61. The method of clause 60, wherein the administration occurs within at least 1 or 2 or 3 weeks of exposure to virulent Porcine Circovirus.

62. The composition for use according to any one of clauses 32 to 41 or the method of any one of clauses 42 to 55, wherein the administration comprises a single, one shot administration; or a single, one dose administration of the composition; and not a multi-shot or multi-dose regimen; or wherein the administration consists of a single, one shot administration; or a single, one dose administration; and not a multi-shot or multi-dose regimen; or wherein the administration comprises a multi-shot or multi-dose regimen of the composition; or wherein the administration comprises a two-shot or two-dose regimen of the composition or wherein the administration consists of a two-shot or two-dose regimen of the composition.

63. Use of a composition of any one of clauses 1 to 31 in a method of any one of clauses 42-55; or use of a PCV3 ORF2 protein, alone or in combination, of any one of the compositions of clauses 1 to 31, for use in the preparation of a composition for inducing an immunological or immune response or a protective immune or immunological response against (i) PCV3 and/or (ii) PCV2 and PCV3 and/or (iii) PCV3 and another porcine pathogen and/or (iv) PCV3, PCV2 and another porcine pathogen, or for use in a method for inducing an immunological or immune response or a protective immune or immunological response against (i) PCV3 and/or (ii) PCV2 and PCV3 and/or (iii) PCV3 and another porcine pathogen and/or (iv) PCV3, PCV2 and another porcine pathogen.

64. A method for preparing a composition as claused in any one of clauses 1 to 31, comprising producing the PCV3 ORF2 protein by a baculovirus expression system in cultured insect cells.

65. The method of clause 64, including inactivating the baculovirus.

66. The method of clause 65, wherein the inactivating comprises heat treatment or use of a virus inactivating agent.

67. The method of clause 66, wherein the virus inactivating agent comprises an aziridine compound.

68. The method of clause 67, wherein the aziridine compound comprises BEI.

69. A recombinant vector comprising a polynucleotide sequence that encodes a polypeptide sequence that encodes a PCV3 ORF2 protein.

70. The recombinant vector of clause 69, wherein the PCV3 ORF2 is from group a1, b1 or b2.

71. A composition comprising a (i) porcine circovirus type 3 (PCV3) ORF2 protein, a parvovirus (PPV) protein and optionally a PRRSV (porcine respiratory and reproductive syndrome virus) protein and (ii) a veterinary-acceptable carrier selected from the group consisting of a solvent, a dispersion media, a coating, a stabilizing agent, a diluent, a preservative, an anti-microbial agent, an antifungal agent, an isotonic agent, an adsorption delaying agent, an adjuvant, cell culture supernatant, a stabilizing agent, a viral vector, an expression vector, an immunomodulatory agent, and/or any combination thereof.

72. The composition of clause 71, wherein the veterinary-acceptable carrier comprises an adjuvant, immunomodulatory agent, cell culture supernatant, viral or expression vector or any combination thereof.

73. The composition of clause 71 or 72, wherein the PPV protein is a PPV VP2 capsid protein.

74. The composition of any one of clauses 71 to 73, wherein the PRRSV protein is a PRRSV ORF4, ORF5, ORF6, or ORF7.

75. The composition of clause 73 or 74, wherein the PPV protein and/or the PRRSV protein is expressed in a vector.

76. The composition of any one of clauses 71 to 75 wherein the composition is an immunogenic composition administered in two doses to a porcine.

77. The composition of clause 76, wherein the porcine is a gilt or a sow.

78. The composition of clause 76 or 77, wherein the administrating is before mating/semination, before pregnancy, during pregnancy or during lactation.

79. The composition of any one of clauses 76-78, wherein the immunogenic composition comprises between 0.1 μg and 150 μg, preferably between 0.25 μg and 75 μg, more preferably between 0.5 μg and 37.5 μg, even more preferably between 0.5 μg and 15 μg, most preferably between 0.5 μg and 6 μg of the PCV3, PPV and/or PRRSV antigen.

80. The composition of any one of clauses 76-79, wherein the immunogenic composition is administered intramuscularly.

81. A method for eliciting an immune response or an immunological response or a protective immune or immunological response against porcine circovirus 3 (PCV3) comprising parenterally or subcutaneously administering to a porcine of a single shot, single administration or single dose (i) at least 2 μg to about 400 μg of a PCV3 ORF2 recombinant protein expressed by a baculovirus system and (ii) a veterinary-acceptable carrier comprising a solvent, a dispersion media, a coating, a stabilizing agent, a diluent, a preservative, an anti-microbial agent, an antifungal agent, an isotonic agent, an adsorption delaying agent, an adjuvant, cell culture supernatant, a stabilizing agent, a viral or expression vector, an immunomodulatory agent and/or any combination thereof.

82. The method of clause 81, wherein the porcine is a piglet, pig or a sow, or a pre-breeding gilt.

83. The method of clause 81 or clause 82, wherein the porcine is about 1 week or 2 weeks or 3 weeks of age or 7-28 or 7-22 or 14-22 or 16-22 or 21+/−5 days of age.

84. The method of any one of clauses 81 to 83, wherein the veterinary-acceptable carrier comprises an adjuvant, immunomodulatory agent, cell culture supernatant, viral or expression vector or any combination thereof.

85. The method of any one of clauses 81 to 84, wherein the PCV3 ORF2 is any phylogenetic clade of PCV3 or from group PCV3a, PCV3a1, PCV3b, PCV3b1, or PCV3b.

86. The method of any one of clauses 81 to 85, wherein the PCV3 ORF2 protein comprises or consists of an amino acid sequence encoded by a polynucleotide sequence having at least 90%, or at least 91%, or at least 92%, or at least 93%, or at least 94%, or at least 95%, or at least 96%, or at least 97%, or at least 98%, or at least 99%, or 100% sequence identity with SEQ ID NO: 1 or sequence homology with SEQ ID NO:1, SEQ ID NO: 6 or SEQ ID NO: 7.

87. The method of any one of clauses 81 to 86, wherein the single shot, single administration or single dose further comprises a PCV2 ORF2 protein or an additional antigen of an additional porcine pathogen.

88. The method of clause 87, wherein the additional antigen of an additional porcine pathogen comprises a PRRSV (porcine respiratory and reproductive syndrome virus) antigen, a Mycoplasma hyopneumoniae bacterin antigen, a Mycoplasma hyopneumoniae supernatant antigen, an Aujeszky's disease or pseudorabies antigen, a swine influenza antigen, a swine fever antigen (classical or African or combination thereof), an Actinobacillus pleuropneumoniae antigen, an Escherichia coli antigen, a porcine parvovirus (PPV) antigen or a Pasteurella multocida antigen, or a combination thereof.

89. The method of any one of clauses 81 to 88, wherein the adjuvant comprises a polymer of acrylic or methacrylic acid; copolymer of maleic anhydride and alkenyl derivative; a polymer of acrylic or methacrylic acid which is cross-linked; a polymer of acrylic or methacrylic acid which is cross-linked with a polyalkenyl ether of sugar or polyalcohol; a carbomer; an acrylic polymer cross-linked with a polyhydroxylated compound having at least 3 and not more than 8 hydroxyl groups with hydrogen atoms of at least three hydroxyls optionally or being replaced by unsaturated aliphatic radicals having at least 2 carbon atoms with said radicals containing from 2 to 4 carbon atoms such as vinyls, allyls and other ethylenically unsaturated groups and the unsaturated radicals may themselves contain other substituents, such as methyl; a carbopol; Carbopol 974P; Carbopol 934P; Carbopol 971P; aluminum hydroxide; aluminum phosphate; a saponin; Quil A; QS-21; GPI-0100; a water-in-oil emulsion; an oil-in-water emulsion; a water-in-oil-in-water emulsion; an emulsion based on light liquid paraffin oil or European Pharmacopea type adjuvant; an isoprenoid oil; squalane; squalene oil resulting from oligomerization of alkenes or isobutene or decene; (an) ester(s) of acid(s) or of alcohol(s) containing a linear alkyl group; plant oil(s); ethyl oleate; propylene glycol di-(caprylate/caprate); glyceryl tri-(caprylate/caprate); propylene glycol dioleate; (an) ester(s) of branched fatty acid(s) or alcohol(s); isostearic acid ester(s); nonionic surfactant(s); (an) ester(s) of sorbitan or of mannide or of glycol or of polyglycerol or of propylene glycol or of oleic, or isostearic acid or of ricinoleic acid or of hydroxystearic acid, optionally ethoxylated, anhydromannitol oleate; polyoxypropylene-polyoxyethylene copolymer blocks, a Pluronic product, RIBI adjuvant system; Block co-polymer; SAF-M; monophosphoryl lipid A; Avridine lipid-amine adjuvant; heat-labile enterotoxin from E. coli (recombinant or otherwise); cholera toxin; IMS 1314, or muramyl dipeptide.

90. The method of any one of clauses 81 to 89, wherein the PCV3 ORF2 protein is present in an amount of 0.2 to about 400 μg/ml, or about 0.3 to about 200 μg/ml, or about 0.35 to about 100 μg/ml, or about 0.4 to about 50 g/ml, or about 0.45 to about 30 μg/ml, or about 0.6 to about 15 μg/ml, or about 0.75 to about 8 μg/ml, or about 1.0 to about 6 μg/ml, or about 1.3 to about 3.0 μg/ml, or about 1.4 to about 2.5 g/ml, or about 1.5 to about 2.0 g/ml, or about 1.6 μg/ml.

91. The method of clause 87, wherein the PCV3 ORF2 protein or total PCV2 and PCV3 ORF2 proteins are present in an amount of about 0.2 to about 400 μg/dose, or about 0.3 to about 200 μg/dose, or about 0.35 to about 100 μg/dose, or about 0.4 to about 50 μg/dose, or about 0.45 to about 30 μg/dose, or about 0.6 to about 15 μg/dose, or about 0.75 to about 8 μg/dose, or about 1.0 to about 6 μg/dose, or about 1.3 to about 3.0 μg/dose, or about 1.4 to about 2.5 μg/dose, or about 1.5 to about 2.0 μg/dose, or about 1.6 μg/dose.

92. The method of any one of clauses 81 to 91, comprising from about 50 μg to about 2000 μg of adjuvant; or wherein adjuvant present in an amount about 250 μg/ml dose of the composition, or wherein the adjuvant is present in an amount of about 100 μg to about 10 mg per dose; or wherein the adjuvant is present in an amount of about 500 μg to about 5 mg per dose; the adjuvant is present in an amount of about 750 μg to about 2.5 mg per dose; or the adjuvant is present in an amount of about 1 mg per dose.

93. The method of any one of clauses 82 to 92, wherein the immunomodulatory agent comprises an interleukin, an interferon or other cytokine.

94. The method of any one of clauses 81 to 93, wherein the single shot, single administration or single dose further comprises from about 1 μg/ml to about 60 μg/ml of antibiotic(s), or less than about 30 μg/ml of an antibiotic.

95. The method of clause 84, wherein the antibiotic comprises Gentamicin.

96. The method of any one of clauses 81 to 95, wherein the single shot, single administration or single dose comprises (i) PCV3 ORF2 protein, (ii) at least a portion of baculovirus that expressed said PCV3 ORF2 protein, (iii) a portion of cell culture of cells that were infected or transfected with recombinant baculovirus that expressed said PCV3 ORF2 protein, (iv) inactivating agent or inactivating agent comprising binary ethyleneimine (BEI), (v) sodium thiosulfate or sodium thiosulfate in equivalent amounts to inactivating agent or BEI; (vi) adjuvant or adjuvant comprising Carbopol or Carbopol 971, and (vii) phosphate salt in a physiologically acceptable concentration.

97. The method of clause 96, wherein about 90% of the components (i) to (iii) have a size smaller than 1 μm and the pH of said composition is adjusted to about 6.5 to 7.5.

98. The method of clause 96 or 97, wherein the BEI is from the cell culture having been treated with about 2 to 8 or about 5 mM BEI to inactivate the baculovirus and/or the composition contains about 2 to 8 or about 5 mM BEI and/or the composition contains about 1 mg of the Carbopol or Carbopol 971.

99. The method of any one of clauses 81 to 98, wherein the method further comprises reducing or preventing clinical signs or disease caused by a PCV3 or porcine epidemic diarrhea virus (PEDV) infection in a pregnant sow or a piglet.

100. The method of clause 99, wherein the reducing or preventing clinical signs or disease in the piglet comprises the piglet suckling a sow administered with the single shot, single administration or single dose.

101. The method of clause 99, wherein the reducing or preventing clinical signs or disease in the piglet comprises administering the single shot, single administration or single dose to the pregnant sow.

102. The method of clause 101, further comprising the piglet suckling the sow after the sow has given birth to the piglet.

103. The method of any one of clauses 99 to 102, wherein the clinical sign is reduction of average daily weight gain, mortality, development, production or expelling of a mummified, stillborn and/or weak fetus, a gross lesion, a histologic lesion, replication of PCV3 in a tissue or PCV3 viremia.

104. The method of any one of clauses 81 to 103, wherein the parenterally or subcutaneously administering is intramuscular or intradermal.

105. A non-naturally occurring PCV3 ORF2protein comprising an engineered FG loop, wherein the FG loop comprises three or fewer positively charged amino acids.

106. The PCV3 ORF2 protein of clause 105, wherein the FG loop comprises two positively charged amino acids.

107. The PCV3 ORF2 protein of clause 105, wherein the FG loop comprises one positively charged amino acid.

108. The PCV3 ORF2 protein of clause 105, wherein the FG loop lacks positively charged amino acids.

109. The PCV3 ORF2 protein of clause 105, wherein the FG loop lacks arginine and lysine residues.

110. The PCV3 ORF2 protein of clause 105, wherein the FG loop lacks arginine, lysine, and histidine residues.

111. The PCV3 ORF2 protein of clause 105, wherein the FG loop comprises QPFSYH, LSRGF, or MASGF.

112. A non-naturally occurring PCV3 ORF2protein comprising an engineered C-terminal extension.

113. The PCV3 ORF2 protein of clause 112, wherein the C-terminal extension comprises from about 1 to about 10, from about 5 to about 20, or from about 10 to about 30 amino acids.

114. The PCV3 ORF2 protein of clause 112, wherein the C-terminal extension comprises from about 1 to about 10, or from about 5 to about 20, or from about 10 to 30 amino acids, about 50 to about 200 amino acids, about 60 to about 190 amino acids, about 70 to about 180 amino acids, about 80 to about 170 amino acids, about 90 to about 160 amino acids or about 100 to about 150 amino acids.

115. The PCV3 ORF2 protein of clause 112, wherein the C-terminal extension comprises C-terminal amino acids from a different capsid protein.

116. The PCV3 ORF2 protein of clause 115, wherein the C-terminal extension comprises C-terminal amino acids from a PCV2 capsid, as BFDV capsid, or a CaCV capsid.

117. The PCV3 ORF2 protein of clause 112, wherein the C-terminal extension comprises EFNLKDPPLN, PK, or QFAPNNPSTEFDYETGRQL.

118. A method of making a self-assembling PCV3 ORF2 capsid protein, which comprises substituting one or more arginine, lysine, or histidine amino acids in the FG loop with non-positively charged amino acids.

119. A method of enhancing self-assembly of a PCV3 ORF2 capsid protein, which comprises adding or inserting amino acid residues at the C-terminal of the protein.

120. The method of clause 118, which comprises adding or inserting from 1 to 10, or from about 5 to about 20, or from about 10 to about 30 amino acids, about 50 to about 200 amino acids, about 60 to about 190 amino acids, about 70 to about 180 amino acids, about 80 to about 170 amino acids, about 90 to about 160 amino acids or about 100 to about 150 amino acids.

121. The method of clause 119, which comprises adding or inserting amino acids from a different capsid protein.

122. The method of clause 121, wherein the added or inserted amino acids are from a PCV2 capsid, as BFDV capsid, or a CaCV capsid.

123. The method of clause 121, wherein the added or inserted amino acids comprise EFNLKDPPLN, PK, or QFAPNNPSTEFDYETGRQL.

124. A composition comprising the PCV protein of any one of clauses 105 to 117 or the protein produced by the method of any one of clauses 118 to 123 in an amount to elicit an immune response or a protective immune response against PCV3 and/or clinical symptoms thereof, from a single administration and a veterinary-acceptable carrier comprising a solvent, a dispersion media, a coating, a stabilizing agent, a diluent, a preservative, an anti-microbial agent, an antifungal agent, an isotonic agent, an adsorption delaying agent, an adjuvant, cell culture supernatant, a stabilizing agent, a viral or expression vector, an immunomodulatory agent and/or any combination thereof. 125. The composition of clause 124, wherein the PCV3 ORF2 protein is encoded by SEQ ID NO: 6 or SEQ ID NO: 7.

126. The composition of clause 124 or 125, wherein the veterinary-acceptable carrier comprises an adjuvant, immunomodulatory agent, cell culture supernatant, viral or expression vector or any combination thereof.

127. The composition of any one of clauses 124 to 126 further comprising a PCV2 ORF2 protein, preferably an antigenic PCV2 ORF2 protein (a PCV2 ORF2 antigen), or an additional antigen of an additional porcine pathogen.

128. The composition of clause 127, wherein the additional antigen of an additional porcine pathogen comprises a PRRSV (porcine respiratory and reproductive syndrome virus) antigen, a Mycoplasma hyopneumoniae bacterin antigen, a Mycoplasma hyopneumoniae supernatant antigen, an Aujeszky's disease or pseudorabies antigen, a swine influenza antigen, a swine fever antigen (classical or African or combination thereof), an Actinobacillus pleuropneumoniae antigen, an Escherichia coli antigen, a porcine parvovirus (PPV) antigen or a Pasteurella multocida antigen, or a combination thereof.

129. The composition of any one of clauses 124 to 128, wherein the adjuvant comprises a polymer of acrylic or methacrylic acid; copolymer of maleic anhydride and alkenyl derivative; a polymer of acrylic or methacrylic acid which is cross-linked; a polymer of acrylic or methacrylic acid which is cross-linked with a polyalkenyl ether of sugar or polyalcohol; a carbomer; an acrylic polymer cross-linked with a polyhydroxylated compound having at least 3 and not more than 8 hydroxyl groups with hydrogen atoms of at least three hydroxyls optionally or being replaced by unsaturated aliphatic radicals having at least 2 carbon atoms with said radicals containing from 2 to 4 carbon atoms such as vinyls, allyls and other ethylenically unsaturated groups and the unsaturated radicals may themselves contain other substituents, such as methyl; a carbopol; Carbopol 974P; Carbopol 934P; Carbopol 971P; aluminum hydroxide; aluminum phosphate; a saponin; Quil A; QS-21; GPI-0100; a water-in-oil emulsion; an oil-in-water emulsion; a water-in-oil-in-water emulsion; an emulsion based on light liquid paraffin oil or European Pharmacopea type adjuvant; an isoprenoid oil; squalane; squalene oil resulting from oligomerization of alkenes or isobutene or decene; (an) ester(s) of acid(s) or of alcohol(s) containing a linear alkyl group; plant oil(s); ethyl oleate; propylene glycol di-(caprylate/caprate); glyceryl tri-(caprylate/caprate); propylene glycol dioleate; (an) ester(s) of branched fatty acid(s) or alcohol(s); isostearic acid ester(s); nonionic surfactant(s); (an) ester(s) of sorbitan or of mannide or of glycol or of polyglycerol or of propylene glycol or of oleic, or isostearic acid or of ricinoleic acid or of hydroxystearic acid, optionally ethoxylated, anhydromannitol oleate; polyoxypropylene-polyoxyethylene copolymer blocks, a Pluronic product, RIBI adjuvant system; Block co-polymer; SAF-M; monophosphoryl lipid A; Avridine lipid-amine adjuvant; heat-labile enterotoxin from E. coli (recombinant or otherwise); cholera toxin; IMS 1314, or muramyl dipeptide.

130. The composition of any one of clauses 124 to 129, wherein the PCV3 ORF2 protein is present in an amount of 0.2 to about 400 μg/ml, or about 0.3 to about 200 μg/ml, or about 0.35 to about 100 μg/ml, or about 0.4 to about 50 μg/ml, or about 0.45 to about 30 μg/ml, or about 0.6 to about 15 μg/ml, or about 0.75 to about 8 μg/ml, or about 1.0 to about 6 μg/ml, or about 1.3 to about 3.0 μg/ml, or about 1.4 to about 2.5 μg/ml, or about 1.5 to about 2.0 μg/ml, or about 1.6 μg/ml.

131. The composition of any one of clauses 124 to 130, wherein the PCV3 ORF2 protein or total PCV2 and PCV3 ORF2 proteins are present in an amount of about 0.2 to about 400 μg/dose, or about 0.3 to about 200 μg/dose, or about 0.35 to about 100 μg/dose, or about 0.4 to about 50 μg/dose, or about 0.45 to about 30 μg/dose, or about 0.6 to about 15 μg/dose, or about 0.75 to about 8 μg/dose, or about 1.0 to about 6 μg/dose, or about 1.3 to about 3.0 μg/dose, or about 1.4 to about 2.5 μg/dose, or about 1.5 to about 2.0 μg/dose, or about 1.6 μg/dose.

132. The composition of any one of clauses 124 to 131, comprising from about 50 μg to about 2000 μg of adjuvant; or wherein adjuvant present in an amount about 250 μg/ml dose of the composition, or wherein the adjuvant is present in an amount of about 100 μg to about 10 mg per dose; or wherein the adjuvant is present in an amount of about 500 μg to about 5 mg per dose; the adjuvant is present in an amount of about 750 μg to about 2.5 mg per dose; or the adjuvant is present in an amount of about 1 mg per dose.

133. The composition of any one of clauses 125 to 132, wherein the immunomodulatory agent comprises an interleukin, an interferon or other cytokine.

134. A vector containing and expressing the PCV protein of any one of clauses 105 to 117 or the protein produced by the method of any one of clauses 118 to 123.

135. The vector of clause 134 wherein the PCV protein is expressed by SEQ ID NO: 6 or SEQ ID NO: 7.

136. The vector of clause 134 or 135, wherein the vector is a baculovirus.

137. A method of preparing the composition of any one of clauses 125 to 133, comprising producing the PCV3 ORF2 protein by a baculovirus expression system in cultured insect cells.

138. The method of clause 137 further comprising inactivating the baculovirus.

139. The method of clause 138, wherein the inactivating comprises heat treatment or use of a virus inactivating agent.

140. The method of clause 139, wherein the virus inactivating agent comprises an aziridine compound.

141. The method of clause 140, wherein the aziridine compound comprises BEI.

### Clause Set C:

Clause Set C—The present invention will now be described by way of the following set of numbered clauses (Clause Set C). The disclosure in this set of clauses is equally applicable to the present invention. Likewise the disclosure in this set of clauses is equally applicable to each of the other set of clauses.

1. A porcine circovirus type 3 (PCV3) antigenic protein, wherein said protein is PCV3 ORF2 protein or a functional antigenic variant thereof.

2. A protein according to clause 1 wherein said PCV3 ORF2 protein is a protein encoded by SEQ ID No. 1.

3. A protein according to clause 1 or clause 2 wherein said protein is a functional antigenic variant of PCV3 ORF2.

4. A protein according to any one of the preceding clauses wherein said protein is a functional antigenic variant of the protein encoded by SEQ ID No. 1.

5. A protein according to any one of the preceding clauses wherein said functional antigenic variant is capable of a higher yield of virus-like particles (VLPs) than the protein encoded by SEQ ID No. 1.

6. A protein according to any one of the preceding clauses wherein said functional antigenic variant is capable of a higher yield of VLPs than the protein encoded by SEQ ID No. 1 as determinable by Western blot analysis.

7. A protein according to any one of the preceding clauses wherein said functional antigenic variant has fewer positive charged amino acid residues than the protein encoded by SEQ ID No. 1.

8. A protein according to any one of the preceding clauses wherein said functional antigenic variant has one or more substitutions in the FG loop of the protein encoded by SEQ ID No. 1.

9. A protein according to any one of the preceding clauses wherein said functional antigenic variant has one or more substitutions in the FG loop of the protein encoded by SEQ ID No. 1, wherein those substitutions comprise substitutions of one or more of the S residue and/or the K residues and/or the H residue of the motif SKKKH of the FG loop of the protein encoded by SEQ ID No. 1.

10. A protein according to any one of the preceding clauses wherein said functional antigenic variant has one or more substitutions in the FG loop of the protein encoded by SEQ ID No. 1, wherein those substitutions comprise substitutions of one or more of the S residue and/or the K residues of the motif SKKKH of the FG loop of the protein encoded by SEQ ID No. 1.

11. A protein according to any one of the preceding clauses wherein said functional antigenic variant has one or more substitutions in the FG loop of the protein encoded by SEQ ID No. 1, wherein those substitutions comprise substitutions of the S residue or H residue and all of the K residues of the motif SKKKH of the FG loop of the protein encoded by SEQ ID No. 1.

12. A protein according to any one of the preceding clauses wherein said functional antigenic variant has one or more substitutions in the FG loop of the protein encoded by SEQ ID No. 1, wherein those substitutions comprise a substitution of at least S and/or H and any K of the motif SKKKH of the FG loop of the protein encoded by SEQ ID No. 1 with Q or P or F or S.

13. A protein according to any one of the preceding clauses wherein said functional antigenic variant has one or more substitutions in the FG loop of the protein encoded by SEQ ID No. 1, wherein those substitutions comprise substitution of the motif SKKK within the motif SKKKH of the FG loop of the protein encoded by SEQ ID No. 1 with QPFS or substitution of the motif KKKH within the motif SKKKH of the FG loop of the protein encoded by SEQ ID No. 1 with QPFS.

14. A protein according to any one of the preceding clauses wherein said functional antigenic variant is encodable by all or part of SEQ ID Nos. 1, 2, 5, 6 or 7.

15. A protein according to any one of the preceding clauses wherein said functional antigenic variant is encoded by all or part of SEQ ID No. 1, 2, 5, 6 or 7.

16. A protein according to any one of the preceding clauses wherein said functional antigenic variant has a C terminal end extends beyond the terminal SVL sequence of the protein encoded by SEQ ID No. 1, preferably wherein said extension is all or includes a sequence from a circoviridae virus, and preferably wherein at least a part of said extension replaces the terminal SVL sequence of the protein encoded by SEQ ID No. 1.

17. A protein according to any one of the preceding clauses wherein said functional antigenic variant has a C terminal end that extends beyond the terminal SVL sequence of the protein encoded by SEQ ID No. 1; and wherein said extension is from 1 to 100 amino acids long.

18. A protein according to any one of the preceding clauses wherein said functional antigenic variant has a C terminal end that extends beyond the terminal SVL sequence of the protein encoded by SEQ ID No. 1; and wherein said extension is from 1 to 50 amino acids long.

19. A protein according to any one of the preceding clauses wherein said functional antigenic variant has a C terminal end that extends beyond the terminal SVL sequence of the protein encoded by SEQ ID No. 1; and wherein said extension is from 1 to 30 amino acids long.

20. A protein according to any one of the preceding clauses wherein said functional antigenic variant has a C terminal end that extends beyond the terminal SVL sequence of the protein encoded by SEQ ID No. 1; wherein said extension is from 1 to 30 amino acids long; and wherein said extension comprises all of part of the sequence VKININLTPPVATSRVPSRALPLRFGCGHR.

21. A protein according to any one of the preceding clauses wherein said functional antigenic variant has a C terminal end that extends beyond the terminal SVL sequence of the protein encoded by SEQ ID No. 1; wherein said extension is from 1 to 30 amino acids long; and wherein said extension all of comprises the sequence VKININLTPPVATSRVPSRALPLRFGCGHR.

22. A protein according to any one of the preceding clauses wherein said functional antigenic variant is encodable by all or part of SEQ ID No. 1, 2, 5, 6 or 7.

23. A protein according to any one of the preceding clauses wherein said functional antigenic variant is encoded by all or part of SEQ ID No. 1, 2, 5, 6 or 7.

24. A protein according to any one of the preceding clauses wherein said protein is recombinant protein having been prepared by recombinant DNA techniques.

25. A protein according to any one of the preceding clauses wherein said protein is baculovirus expressed protein.

26. A protein according to any one of the preceding clauses wherein said PCV3 is selected from the group consisting of PCV3a and PCV3b.

27. A protein according to any one of the preceding clauses wherein said PCV3 is any phylogenetic clade of PCV3 or selected from the group consisting PCV3a1, PCV3b1, PCV3b2 and PCV3c.

28. A protein according to any one of the preceding clauses wherein said PCV3 ORF2 is from group a1, b1 or b2 (using the subtyping designation of Fux et al., “Full genome characterization of porcine circovirus type 3 isolates reveals the existence of two distinct groups of virus strains,” Virology Journal (2018) 15:25, DOI 10.1186/s12985-018-0929-3 (incorporated herein by reference); see, e.g., Table 4).

29. A protein according to any one of the preceding clauses wherein said PCV3 ORF2 protein comprises or consists of an amino acid sequence encoded by a polynucleotide sequence having at least 90%, or at least 91%, or at least 92%, or at least 93%, or at least 94%, or at least 95%, or at least 96%, or at least 97%, or at least 98%, or at least 99%, or 100% sequence identity with SEQ ID NO: 1 or sequence homology with SEQ ID NO: 1.

30. A protein according to any one of the preceding clauses wherein said variant protein comprises or consists of an amino acid sequence encoded by a polynucleotide sequence having at least 90%, or at least 91%, or at least 92%, or at least 93%, or at least 94%, or at least 95%, or at least 96%, or at least 97%, or at least 98%, or at least 99%, or 100% sequence identity with SEQ ID NO: 1 or sequence homology with SEQ ID NO:6.

31. A protein according to any one of the preceding clauses wherein said variant protein comprises or consists of an amino acid sequence encoded by a polynucleotide sequence having at least 90%, or at least 91%, or at least 92%, or at least 93%, or at least 94%, or at least 95%, or at least 96%, or at least 97%, or at least 98%, or at least 99%, or 100% sequence identity with SEQ ID NO:1 or sequence homology with SEQ ID NO:7.

32. A protein according to any one of the preceding clauses wherein said PCV3 ORF2 protein comprises or consists of an amino acid sequence having at least 90%, or at least 91%, or at least 92%, or at least 93%, or at least 94%, or at least 95%, or at least 96%, or at least 97%, or at least 98%, or at least 99%, or 100% sequence identity with the sequence of SEQ ID NO: 3, 4, 8, 9 or 10 and/or wherein the protein is a recombinant protein.

33 A protein according to any one of the preceding clauses wherein said variant protein comprises or consists of an amino acid sequence having at least 90%, or at least 91%, or at least 92%, or at least 93%, or at least 94%, or at least 95%, or at least 96%, or at least 97%, or at least 98%, or at least 99%, or 100% sequence identity with the sequence of SEQ ID NO: 3, 4, 8, 9 or 10 and/or wherein the protein is a recombinant protein; or a protein according to any one of the preceding clauses wherein said variant protein comprises or consists of an amino acid sequence having at least 90%, or at least 91%, or at least 92%, or at least 93%, or at least 94%, or at least 95%, or at least 96%, or at least 97%, or at least 98%, or at least 99%, or 100% sequence identity with the sequence of SEQ ID NO: 3, 4, 8, 9 or 10, and/or wherein the protein is a recombinant protein, and wherein said protein has one or more substitutions in the FG loop.

34. A protein according to any one of the preceding clauses wherein said variant protein comprises or consists of an amino acid sequence having at least 90%, or at least 91%, or at least 92%, or at least 93%, or at least 94%, or at least 95%, or at least 96%, or at least 97%, or at least 98%, or at least 99%, or 100% sequence identity with the sequence of SEQ ID NO: 3, 4, 8, 9 or 10 and/or wherein the protein is a recombinant protein; or a protein according to any one of the preceding clauses wherein said variant protein comprises or consists of an amino acid sequence having at least 90%, or at least 91%, or at least 92%, or at least 93%, or at least 94%, or at least 95%, or at least 96%, or at least 97%, or at least 98%, or at least 99%, or 100% sequence identity with the sequence of SEQ ID NO: 3, 4, 8, 9 or 10, and/or wherein the protein is a recombinant protein, and wherein said protein has a C terminal end that extends beyond the terminal SVL sequence of the protein encoded by SEQ ID No. 1.

35. A protein according to any one of the preceding clauses wherein said protein is a recombinant protein from expression thereof by an expression vector, comprising a polynucleotide sequence that encodes the protein.

36. A protein according to any one of the preceding clauses wherein said protein is a recombinant protein from expression thereof by a baculovirus expression vector, comprising a polynucleotide sequence that encodes the protein.

37. A nucleotide sequence encoding the protein according to any of the preceding clauses.

38. A vector comprising the nucleotide sequence of any of the preceding clauses.

39. A recombinant vector comprising the nucleotide sequence of any of the preceding clauses.

40. An expression host transformed or transfected with the nucleotide sequence of any of the preceding clauses.

41. A baculovirus expression host transformed or transfected with the nucleotide sequence of any of the preceding clauses.

42. A method of preparing a protein according to any one of the preceding clauses comprising expressing a nucleotide sequence according to any of the preceding clauses.

43. A method of preparing a protein according to any one of the preceding clauses comprising expressing a vector according to any of the preceding clauses.

44. A method of preparing a protein according to any one of the preceding clauses comprising expressing a recombinant vector according to any of the preceding clauses.

45. A method of preparing a protein according to any one of the preceding clauses comprising culturing the expression host according to any of the preceding clauses to cause expression of the protein.

46. A method of preparing a protein according to any one of the preceding clauses comprising transfecting an expression host with the nucleotide sequence of vector according to any one of the preceding clauses and culturing the expression host to cause expression of the protein.

47. A method of preparing a protein according to any one of the preceding clauses comprising culturing the baculovirus expression host according to any of the preceding clauses to cause expression of the protein.

48. A method of preparing a protein according to any one of the preceding clauses comprising transfecting a baculovirus expression host with the nucleotide sequence of vector according to any one of the preceding clauses and culturing the baculovirus expression host to cause expression of the protein.

49. A method according to any one of the preceding clauses wherein an inactivating agent is used when sufficient levels of expressed protein have been achieved.

50. A method according to any one of the preceding clauses wherein an inactivating agent comprising binary ethyleneimine (BEI) is used when sufficient levels of expressed protein have been achieved.

51. A method of preparing a protein according to any one of the preceding clauses comprising transfecting a baculovirus expression host with the nucleotide sequence of vector according to any one of the preceding clauses and culturing the baculovirus expression host in a medium to cause expression of the protein; wherein the medium post expression of the protein comprises (i) said protein, (ii) at least a portion of baculovirus that expressed said protein, (iii) a portion of cell culture of cells that were infected or transfected with recombinant baculovirus that expressed said protein.

52. A method of preparing a protein according to any one of the preceding clauses comprising transfecting a baculovirus expression host with the nucleotide sequence of vector according to any one of the preceding clauses and culturing the baculovirus expression host in a medium to cause expression of the protein; wherein the medium post expression of the protein comprises (i) said protein, (ii) at least a portion of baculovirus that expressed said protein, (iii) a portion of cell culture of cells that were infected or transfected with recombinant baculovirus that expressed said protein; and wherein about 90% of the components (i) to (iii) have a size smaller than 1 μm.

53. A method of preparing a protein according to any one of the preceding clauses comprising transfecting a baculovirus expression host with the nucleotide sequence of vector according to any one of the preceding clauses and culturing the baculovirus expression host in a medium to cause expression of the protein; wherein the medium post expression of the protein comprises (i) said protein, (ii) at least a portion of baculovirus that expressed said protein, (iii) a portion of cell culture of cells that were infected or transfected with recombinant baculovirus that expressed said protein; and wherein about 90% of the components (i) to (iii) have a size smaller than 1 μm and the pH of said composition is adjusted to about 6.5 to 7.5.

54. A method of preparing a protein according to any one of the preceding clauses comprising producing the protein by a baculovirus expression system in cultured insect cells.

55. A method of preparing a protein according to any one of the preceding clauses comprising producing the protein by a baculovirus expression system in cultured insect cells; and wherein the method includes the step of inactivating the baculovirus.

56. A method of preparing a protein according to any one of the preceding clauses comprising producing the protein by a baculovirus expression system in cultured insect cells; and wherein the method includes the step of inactivating the baculovirus; and wherein inactivating step comprises heat treatment or use of a virus inactivating agent.

57. A method of preparing a protein according to any one of the preceding clauses comprising producing the protein by a baculovirus expression system in cultured insect cells; and wherein the method includes the step of inactivating the baculovirus; and wherein inactivating step comprises heat treatment or use of a virus inactivating agent; and wherein the virus inactivating agent comprises an aziridine compound.

58. A method of preparing a protein according to any one of the preceding clauses comprising producing the protein by a baculovirus expression system in cultured insect cells; and wherein the method includes the step of inactivating the baculovirus; and wherein inactivating step comprises heat treatment or use of a virus inactivating agent; and wherein the virus inactivating agent comprises an aziridine compound; wherein the aziridine compound comprises BEI.

59. A protein obtainable by the method according to any one of the preceding clauses.

60. A composition comprising the protein obtainable by the method according to any one of the preceding clauses.

61. A composition obtainable by the method according to any one of the preceding clauses.

62. A composition comprising a protein according to any one of the preceding clauses and a carrier, diluent or excipient.

63. A composition comprising a protein according to any one of the preceding clauses and a veterinary-acceptable carrier, diluent or excipient.

64. A composition according to any one of the preceding clauses wherein the protein is present in an amount of 0.2 to about 400 μg/ml, or 2 to about 400 μg/ml, or 4 to about 400 μg/ml, or 8 to about 400 μg/ml, or about 0.3 to about 200 μg/ml, or 2 to about 200 μg/ml, or 4 to about 200 μg/ml, or 8 to about 200 μg/ml, or about 0.35 to about 100 μg/ml, or 2 to about 100 μg/ml, or 4 to about 100 μg/ml, or 8 to about 100 μg/ml, or about 0.4 to about 50 μg/ml, or about 0.45 to about 30 μg/ml, or about 0.6 to about 15 μg/ml, or about 0.75 to about 8 μg/ml, or about 1.0 to about 6 μg/ml, or about 1.3 to about 3.0 μg/ml, or about 1.4 to about 2.5 μg/ml, or about 1.5 to about 2.0 μg/ml, or about 1.6 μg/ml.

65. A composition comprising a protein according to any one of the preceding clauses wherein the composition comprises any one or more of a solvent, a dispersion media, a coating, a stabilizing agent, a diluent, a preservative, an anti-microbial agent, an antifungal agent, an isotonic agent, an adsorption delaying agent, an adjuvant, cell culture supernatant, a stabilizing agent, a viral vector, an expression vector, and/or an immunomodulatory agent.

66. A composition according to any one of the preceding clauses wherein the carrier, diluent or excipient is any one or more of an adjuvant, immunomodulatory agent, cell culture supernatant, viral or expression vector or any combination thereof.

67. A composition according to any one of the preceding clauses wherein the carrier, diluent or excipient comprises an adjuvant.

68. A composition according to any one of the preceding clauses wherein the carrier, diluent or excipient comprises an adjuvant; wherein the adjuvant comprises one or more of a polymer of acrylic or methacrylic acid; copolymer of maleic anhydride and alkenyl derivative; a polymer of acrylic or methacrylic acid which is cross-linked; a polymer of acrylic or methacrylic acid which is cross-linked with a polyalkenyl ether of sugar or polyalcohol; a carbomer; an acrylic polymer cross-linked with a polyhydroxylated compound having at least 3 and not more than 8 hydroxyl groups with hydrogen atoms of at least three hydroxyls optionally or being replaced by unsaturated aliphatic radicals having at least 2 carbon atoms with said radicals containing from 2 to 4 carbon atoms such as vinyls, allyls and other ethylenically unsaturated groups and the unsaturated radicals may themselves contain other substituents, such as methyl; a carbopol; Carbopol 974P; Carbopol 934P; Carbopol 971P; aluminum hydroxide; aluminum phosphate; a saponin; Quil A; QS-21; GPI-0100; a water-in-oil emulsion; an oil-in-water emulsion; a water-in-oil-in-water emulsion; an emulsion based on light liquid paraffin oil or European Pharmacopea type adjuvant; an isoprenoid oil; squalane; squalene oil resulting from oligomerization of alkenes or isobutene or decene; (an) ester(s) of acid(s) or of alcohol(s) containing a linear alkyl group; plant oil(s); ethyl oleate; propylene glycol di-(caprylate/caprate); glyceryl tri-(caprylate/caprate); propylene glycol dioleate; (an) ester(s) of branched fatty acid(s) or alcohol(s); isostearic acid ester(s); nonionic surfactant(s); (an) ester(s) of sorbitan or of mannide or of glycol or of polyglycerol or of propylene glycol or of oleic, or isostearic acid or of ricinoleic acid or of hydroxystearic acid, optionally ethoxylated, anhydromannitol oleate; polyoxypropylene-polyoxyethylene copolymer blocks, a Pluronic product, RIBI adjuvant system; Block co-polymer; SAF-M; monophosphoryl lipid A; Avridine lipid-amine adjuvant; heat-labile enterotoxin from E. coli (recombinant or otherwise); cholera toxin; IMS 1314, or muramyl dipeptide.

69. A composition according to any one of the preceding clauses wherein the carrier, diluent or excipient comprises an adjuvant; wherein the adjuvant comprises Carbopol or Carbopol 971.

70. A composition according to any one of the preceding clauses wherein the carrier, diluent or excipient comprises an adjuvant; wherein the adjuvant is present in an amount from about 50 μg to about 2000 of the composition; or wherein adjuvant is present in an amount about 250 μg/ml dose of the composition, or wherein the adjuvant is present in an amount of about 100 μg to about 10 mg of the composition; or wherein the adjuvant is present in an amount of about 500 μg to about 5 mg of the composition; the adjuvant is present in an amount of about 750 μg to about 2.5 mg of the composition; or the adjuvant is present in an amount of about 1 mg of the composition.

71. A composition according to any one of the preceding clauses wherein the composition comprises an immunomodulatory agent.

72. A composition according to any one of the preceding clauses wherein the composition comprises an immunomodulatory agent; and wherein the immunomodulatory agent is any one or more of interleukin(s), interferon(s), or other cytokine(s).

73. A composition according to any one of the preceding clauses wherein the composition comprises an antibiotic(s).

74. A composition according to any one of the preceding clauses wherein the composition comprises an antibiotic(s); wherein the antibiotic(s) comprise Gentamicin.

75. A composition according to any one of the preceding clauses wherein the composition comprises an antibiotic(s); and wherein the composition comprises from about 1 μg/ml to about 60 μg/ml of antibiotic(s).

76. A composition according to any one of the preceding clauses wherein the composition comprises an antibiotic(s); and wherein the composition comprises from about 1 μg/ml to less than about 30 μg/ml of antibiotic(s).

77. A composition according to any one of the preceding clauses wherein the composition comprises an additional antigen.

78. A composition according to any one of the preceding clauses wherein the composition comprises an additional antigen; wherein said additional antigen is not a PCV3 ORF2 antigen.

79. A composition according to any one of the preceding clauses wherein the composition comprises an additional antigen; wherein said additional antigen is not a PCV3 antigen.

80. A composition according to any one of the preceding clauses wherein the composition comprises an additional antigen of an additional porcine pathogen.

81. A composition according to any one of the preceding clauses wherein the composition further comprises an antigen of an additional porcine pathogen, wherein said pathogen is any one or more of PCV2, PRRSV (porcine respiratory and reproductive syndrome virus) antigen, a Mycoplasma hyopneumoniae bacterin antigen, a Mycoplasma hyopneumoniae supernatant antigen, an Aujeszky's disease or pseudorabies antigen, a swine influenza antigen, a swine fever antigen (classical or African or combination thereof), an Actinobacillus pleuropneumoniae antigen, an Escherichia coli antigen, a porcine parvovirus (PPV) antigen or a Pasteurella multocida antigen.

82. A composition according to any one of the preceding clauses wherein the composition further comprises an antigen of an additional porcine pathogen, wherein said composition further comprises one or more of an antigen of PCV2, an antigen of a PRRSV and an antigen of a PPV.

83. A composition according to any one of the preceding clauses wherein the composition further comprises an antigen of PCV2.

84. A composition according to any one of the preceding clauses wherein the composition further comprises an antigen of PCV2; wherein PCV2 antigen is PCV2 ORF2 protein.

85. A composition according to any one of the preceding clauses wherein the composition further comprises an antigen of PCV2; wherein PCV2 antigen is recombinant PCV2 ORF2 protein.

86. A composition according to any one of the preceding clauses wherein the composition further comprises an antigen of PCV2; wherein PCV2 antigen is recombinant baculovirus expressed PCV2 ORF2 protein.

87. A composition according to any one of the preceding clauses wherein the composition is in a dosage form.

88. A composition according to any one of the preceding clauses wherein the composition is formulated and/or packaged for a single dose or one shot administration.

89. A composition according to any one of the preceding clauses wherein the composition is formulated and/or packaged for a multi-dose regimen.

90. A composition according to any one of the preceding clauses wherein the composition is formulated and/or packaged for a two-dose regimen.

91. A composition according to any one of the preceding clauses wherein the composition is in a dosage form; and wherein said dosage form is delivered from a container containing a larger amount of said composition and wherein a dosage form of said composition is capable of being delivered from said container.

92. A composition according to any one of the preceding clauses wherein the composition is in a dosage form; and wherein said dosage form is delivered from a container containing a larger amount of said composition and wherein a dosage form of said composition is capable of being delivered from said container; and wherein said container contains at least 10 doses of said composition.

93. A composition according to any one of the preceding clauses wherein the composition is in a dosage form; and wherein said dosage form is delivered from a container containing a larger amount of said composition and wherein a dosage form of said composition is capable of being delivered from said container; and wherein said container contains at least 50 doses of said composition.

94. A composition according to any one of the preceding clauses wherein the composition is in a dosage form; and wherein said dosage form is delivered from a container containing a larger amount of said composition and wherein a dosage form of said composition is capable of being delivered from said container; and wherein said container contains at least 100 doses of said composition.

95. A composition according to any one of the preceding clauses wherein the composition is in a dosage form; and wherein said dosage form is delivered from a container containing a larger amount of said composition and wherein a dosage form of said composition is capable of being delivered from said container; and wherein said container contains at least 200 doses of said composition.

96. A composition according to any one of the preceding clauses wherein the composition is in a dosage form; and wherein said dosage form is delivered from a container containing a larger amount of said composition and wherein a dosage form of said composition is capable of being delivered from said container; and wherein said container contains at least 250 doses of said composition.

97. A composition according to any one of the preceding clauses wherein the composition comprises an antigen of PCV2; wherein PCV2 antigen is recombinant baculovirus expressed PCV2 ORF2 protein; and wherein either the protein or combined total amount of the PCV3 ORF2 protein and PCV2 ORF protein are present in an amount of about 0.2 to about 400 μg/dose, or 2 to about 400 μg/dose, or 4 to about 400 μg/dose, or 8 to about 400 μg/dose, or about 0.3 to about 200 μg/dose, or 2 to about 200 μg/dose, or 4 to about 200 μg/dose, or 8 to about 200 μg/dose, or about 0.35 to about 100 μg/dose, or 2 to about 100 μg/dose, or 4 to about 100 μg/dose, or 8 to about 100 μg/dose, or about 0.4 to about 50 μg/dose, or about 0.45 to about 30 μg/dose, or about 0.6 to about 15 μg/dose, or about 0.75 to about 8 μg/dose, or about 1.0 to about 6 μg/dose, or about 1.3 to about 3.0 μg/dose, or about 1.4 to about 2.5 μg/dose, or about 1.5 to about 2.0 μg/dose, or about 1.6 μg/dose.

98. A composition according to any one of the preceding clauses wherein the composition comprises a salt.

99. A composition according to any one of the preceding clauses wherein the composition comprises an inactivated viral vector and/or cell culture supernate.

100. A composition according to any one of the preceding clauses wherein the composition comprises an inactivated viral vector and cell culture supernate.

101. A composition according to any one of the preceding clauses wherein the composition comprises (i) the protein, (ii) at least a portion of baculovirus that expressed said protein, (iii) a portion of cell culture of cells that were infected or transfected with recombinant baculovirus that expressed said protein, (iv) inactivating agent or inactivating agent comprising binary ethyleneimine (BEI), (v) sodium thiosulfate or sodium thiosulfate in equivalent amounts to inactivating agent or BEI; (vi) adjuvant or adjuvant comprising Carbopol or Carbopol 971, and (vii) phosphate salt in a physiologically acceptable concentration.

102. A composition according to any one of the preceding clauses wherein the composition comprises (i) the protein, (ii) at least a portion of baculovirus that expressed said protein, (iii) a portion of cell culture of cells that were infected or transfected with recombinant baculovirus that expressed said protein, (iv) inactivating agent or inactivating agent comprising binary ethyleneimine (BEI), (v) sodium thiosulfate or sodium thiosulfate in equivalent amounts to inactivating agent or BEI; (vi) adjuvant or adjuvant comprising Carbopol or Carbopol 971, and (vii) phosphate salt in a physiologically acceptable concentration; and wherein the BEI is from the cell culture having been treated with about 2 to 8 or about 5 mM BEI to inactivate the baculovirus and/or the composition contains about 2 to 8 or about 5 mM BEI and/or the composition contains about 1 mg of the Carbopol or Carbopol 971.

103. A composition according to any one of the preceding clauses wherein the composition is an immunogenic composition comprising a protein according to any one of the preceding clauses and a carrier, diluent or excipient.

104. A composition according to any one of the preceding clauses wherein the composition is an immunogenic composition comprising a protein according to any one of the preceding clauses and a carrier, diluent or excipient; and an additional antigen according to any one of the preceding clauses.

105. A process of making the composition according to any one of the preceding clauses wherein the protein according to any one of the preceding clauses is admixed with the carrier, diluent or excipient.

106. A process of making the composition according to any one of the preceding clauses wherein the protein according to any one of the preceding clauses is admixed with the carrier, diluent or excipient; and the additional antigen.

107. A protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses for use as a medicament.

108. A protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses for use as a vaccine.

109. A protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses for use in a method for eliciting an immune response or an immunological response or a protective immune or immunological response against PCV3 in an animal.

110. A protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses for use in a method for eliciting an immune response or an immunological response or a protective immune or immunological response against PCV3 in swine.

111. A protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses for use in a method for eliciting an immune response or an immunological response or a protective immune or immunological response against PCV3 in pigs.

112. A protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses for use in a method for eliciting an immune response or an immunological response or a protective immune or immunological response against PCV3 in piglets.

113. A protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses for use in a method for eliciting an immune response or an immunological response or a protective immune or immunological response against PCV3 in piglets; wherein the piglets are to be suckled by sows to which the protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses has been administered.

114. A protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses for use in a method for eliciting an immune response or an immunological response or a protective immune or immunological response against PCV3 in sows.

115. A protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses for use in a method for eliciting an immune response or an immunological response or a protective immune or immunological response against PCV3 in pregnant sows, gilts or pre-breeding gilts.

116. A protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses for use in inducing an immune response against PCV3 in animals.

117. A protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses for use in inducing an immune response against PCV3 in swine.

118. A protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses for use in inducing an immune response against PCV3 in pigs.

119. A protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses for use in inducing an immune response against PCV3 in piglets.

120. A protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses for use in inducing an immune response against PCV3 in piglets; wherein the piglets are to be suckled by sows to which the protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses has been administered.

121. A protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses for use in inducing an immune response against PCV3 in sows.

122. A protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses for use in inducing an immune response against PCV3 in pregnant sows, gilts or pre-breeding gilts.

123. A protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses for use in a method of reducing or preventing the clinical signs or clinical symptoms or disease caused by an infection with PCV3 in an animal or for use in a method of treating or preventing an infection with PCV3 in an animal.

124. A protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses for use in a method of reducing or preventing the clinical signs or clinical symptoms or disease caused by an infection with PCV3 in an animal or for use in a method of treating or preventing an infection with PCV3 in an animal; wherein said animal is swine.

125. A protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses for use in a method of reducing or preventing the clinical signs or clinical symptoms or disease caused by an infection with PCV3 in an animal or for use in a method of treating or preventing an infection with PCV3 in an animal; wherein said animal is a pig.

126. A protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses for use in a method of reducing or preventing the clinical signs or clinical symptoms or disease caused by an infection with PCV3 in an animal or for use in a method of treating or preventing an infection with PCV3 in an animal; wherein said animal is a piglet.

127. A protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses for use in a method of reducing or preventing the clinical signs or clinical symptoms or disease caused by an infection with PCV3 in an animal or for use in a method of treating or preventing an infection with PCV3 in an animal; wherein said animal is a piglet; and wherein the piglet is to be suckled by a sow to which the protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses has been administered.

128. A protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses for use in a method of reducing or preventing the clinical signs or disease caused by an infection with PCV3 in an animal or for use in a method of treating or preventing an infection with PCV3 in an animal; wherein said animal is a sow.

129. A protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses for use in a method of reducing or preventing the clinical signs or clinical symptoms or disease caused by an infection with PCV3 in an animal or for use in a method of treating or preventing an infection with PCV3 in an animal; wherein said animal is a pregnant sow, gilt or pre-breeding gilt.

130. A protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses for use in immunizing an animal against PCV3.

131. A protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses for use in immunizing an animal against PCV3; wherein said animal is swine.

132. A protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses for use in immunizing an animal against PCV3; wherein said animal is a pig.

133. A protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses for use in immunizing an animal against PCV3; wherein said animal is a piglet.

134. A protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses for use in immunizing an animal against PCV3; wherein said animal is a piglet; and wherein the piglet is to be suckled by a sow to which the protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses has been administered.

135. A protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses for use in immunizing an animal against PCV3; wherein said animal is a sow.

136. A protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses for use in immunizing an animal against PCV3; wherein said animal is a pregnant sow, gilt or pre-breeding gilt.

137. A protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses for use in a method of reducing or eliminating or abrogating PCV3 viral expression in an animal.

138. A protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses for use in a method of reducing or eliminating or abrogating PCV3 viral expression in an animal; wherein said animal is swine.

139. A protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses for use in a method of reducing or eliminating or abrogating PCV3 viral expression in an animal; wherein said animal is a pig.

140. A protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses for use in a method of reducing or eliminating or abrogating PCV3 viral expression in an animal; wherein said animal is a piglet.

141. A protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses for use in a method of reducing or eliminating or abrogating PCV3 viral expression in an animal; wherein said animal is a piglet; and wherein the piglet is to be suckled by a sow to which the protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses has been administered.

141. A protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses for use in a method of reducing or eliminating or abrogating PCV3 viral expression in an animal; wherein said animal is a sow.

142. A protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses for use in a method of reducing or eliminating or abrogating PCV3 viral expression in an animal; wherein said animal is a pregnant sow, gilt or pre-breeding gilt.

143. A protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses for use in inducing the production of antibodies specific for PCV3 in an animal.

144. A protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses for use in inducing the production of antibodies specific for PCV3 in an animal; wherein said animal is swine.

145. A protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses for use in inducing the production of antibodies specific for PCV3 in an animal; wherein said animal is a pig.

146. A protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses for use in inducing the production of antibodies specific for PCV3 in an animal; wherein said animal is a piglet.

147. A protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses for use in inducing the production of antibodies specific for PCV3 in an animal; wherein said animal is a piglet; and wherein the piglet is to be suckled by a sow to which the protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses has been administered.

148. A protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses for use in inducing the production of antibodies specific for PCV3 in an animal; wherein said animal is a sow.

149. A protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses for use in inducing the production of antibodies specific for PCV3 in an animal; wherein said animal is a pregnant sow, gilt or pre-breeding gilt.

150. A protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses for the use according to any one of the preceding clauses wherein said protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses is administered intramuscularly or intradermally to said animal.

151. A protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses for the use according to any one of the preceding clauses wherein said protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses is administered to said animal in conjunction with another antigen, preferably wherein the other pathogen is an antigen to a porcine pathogen.

152. A protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses for the use according to any one of the preceding clauses wherein said protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses is administered to said animal in conjunction with another antigen; wherein said other antigen is not a PCV3 ORF2 antigen, preferably wherein the other pathogen is an antigen to a porcine pathogen.

153. A protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses for the use according to any one of the preceding clauses wherein said protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses is administered to said animal in conjunction with another antigen; wherein said other antigen is not a PCV3 antigen, preferably wherein the other pathogen is an antigen to a porcine pathogen.

154. A protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses for the use according to any one of the preceding clauses wherein said animal is a sow pregnant with a piglet.

155. A protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses for the use according to any one of the preceding clauses wherein said animal is a sow pregnant with a piglet; and wherein the piglet is to be suckled by a sow to which the protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses has been administered.

156. A protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses for the use according to any one of the preceding clauses wherein said animal is a sow; and wherein said protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses is administered twice to said sow.

157. A protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses for the use according to any one of the preceding clauses wherein said animal is a sow; and wherein said protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses is only administered twice to said sow.

158. A protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses for the use according to any one of the preceding clauses wherein said animal is a piglet; and wherein the protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses is administered once to said piglet.

159. A protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses for the use according to any one of the preceding clauses wherein said animal is a piglet; and wherein the protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses is only administered once to said piglet.

160. A protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses for the use according to any one of the preceding clauses wherein said animal is a sow; and wherein the protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses is administered twice to said sow; and wherein said use does not include the administration of any other PCV3 antigen to said animal before or during the administration of said protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses.

161. A protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses for the use according to any one of the preceding clauses wherein said animal is a sow; and wherein the protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses is administered twice to said sow; and wherein said use does not include the administration of any other PCV3 antigen to said animal before or during the administration of said protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses.

162. A protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses for the use according to any one of the preceding clauses wherein said animal is a sow; and wherein the protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses is administered only twice to said sow; and wherein said use does not include the administration of any other PCV3 antigen to said animal before or during the administration of said protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses.

163. A protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses for the use according to any one of the preceding clauses wherein said animal is a piglet; and wherein the protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses is administered once to said piglet; and wherein said use does not include the administration of any other PCV3 antigen to said animal before or during the administration of said protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses.

164. A protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses for the use according to any one of the preceding clauses wherein said animal is a piglet; and wherein the protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses is administered once to said piglet; and wherein said use does not include the administration of any other PCV3 antigen to said animal before or during the administration of said protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses.

165. A protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses for the use according to any one of the preceding clauses wherein said animal is a piglet; and wherein the protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses is administered only once to said piglet; and wherein said use does not include the administration of any other PCV3 antigen to said animal before or during the administration of said protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses.

166. A protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses for the use according to any one of the preceding clauses wherein the administration to the animal in the use consists of a single, one shot administration or a single, one dose administration of said protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses.

167. A protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses for the use according to any one of the preceding clauses wherein the administration to the animal in the use consists of a multi-shot or multi-dose regimen of said protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses.

168. A protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses for the use according to any one of the preceding clauses wherein the administration to the animal in the use consists of a double shot administration; or a dual dose administration of said protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses.

169. A protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses for the use according to any one of the preceding clauses wherein the administration to the animal occurs within at least 1 or 2 or 3 weeks of exposure to virulent Porcine Circovirus.

170. A protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses for the use according to any one of the preceding clauses wherein the animal is a piglet not older than 15 weeks of age, or not older than 6 weeks of age, or not older than 3 weeks of age, or not older than 2 weeks of age, or not older than 1 week of age.

171. A protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses for the use according to any one of the preceding clauses wherein said protein according to any one of the preceding clauses or said composition according to any one of the preceding clauses is for the use of any one of the preceding clauses.

172. A protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses for the use according to any one of the preceding clauses wherein said protein according to any one of the preceding clauses is for the use of any one of the preceding clauses.

173. A protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses for the use according to any one of the preceding clauses wherein said composition according to any one of the preceding clauses is for the use of any one of the preceding clauses.

174. A protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses for the use according to any one of the preceding clauses wherein a second antigen is administered to the animal before administration of the protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses.

175. A protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses for the use according to any one of the preceding clauses wherein a second antigen is administered to the animal at the same time as administration of the protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses.

176. A protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses for the use according to any one of the preceding clauses wherein a second antigen is administered to the animal at the same time and in the same composition as administration of the protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses.

177. A protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses for the use according to any one of the preceding clauses wherein a second antigen is administered to the animal at the same time and in a different composition as administration of the protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses.

178. A protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses for the use according to any one of the preceding clauses wherein a second antigen is administered to the animal after the administration of the protein according to any one of the preceding clauses, or a nucleotide sequence according to any one of the preceding clauses, or an expression vector according to any one of the preceding clauses, or an expression host according to any one of the preceding clauses, or a composition according to any one of the preceding clauses.

179. A protein according to any one of the preceding clauses as the single PCV3 antigen for use in the vaccination of a pig to lessen the severity of clinical signs or clinical symptoms resulting from PCV3 infection in the pig, wherein the protein is in an immunogenic composition that is administered in one dose to the pig.

180. A protein according to any one of the preceding clauses as the single PCV3 antigen for use in the vaccination of a pig to lessen the severity of clinical signs or clinical symptoms resulting from PCV3 infection in the pig, wherein the protein is in an immunogenic composition that is administered in only one dose to the pig.

181. A protein according to any one of the preceding clauses as the single PCV3 antigen for use in the vaccination of a pig to lessen the severity of clinical signs or clinical symptoms resulting from PCV3 infection in the pig, wherein the protein is in an immunogenic composition that is administered in two doses to the pig.

182. A protein according to any one of the preceding clauses as the single PCV3 antigen for use in the vaccination of a pig to lessen the severity of clinical signs or clinical symptoms resulting from PCV3 infection in the pig, wherein the protein is in an immunogenic composition that is administered in only two doses to the pig.

183. A protein according to any one of the preceding clauses for use as the single PCV3 antigen for vaccination of a pig to lessen the severity of clinical signs or clinical symptoms resulting from PCV3 infection in the pig, wherein the protein is in an immunogenic composition that is administered in one dose to the pig.

184. A protein according to any one of the preceding clauses for use as the single PCV3 antigen for vaccination of a pig to lessen the severity of clinical signs or clinical symptoms resulting from PCV3 infection in the pig, wherein the protein is in an immunogenic composition that is administered in only one dose to the pig.

185. A protein according to any one of the preceding clauses for use as the single PCV3 antigen for vaccination of a pig to lessen the severity of clinical signs or clinical symptoms resulting from PCV3 infection in the pig, wherein the protein is in an immunogenic composition that is administered in two doses to the pig.

186. A protein according to any one of the preceding clauses for use as the single PCV3 antigen for vaccination of a pig to lessen the severity of clinical signs or clinical symptoms resulting from PCV3 infection in the pig, wherein the protein is in an immunogenic composition that is administered in only two doses to the pig.

187. An immunogenic composition according to any one of the preceding clauses for lessening the severity of clinical signs or clinical symptoms resulting from PCV3 infection in a pig;
wherein one dose of the immunogenic composition is administered to the pig in a vaccination method to lessen the severity of clinical signs or clinical symptoms resulting from PCV3 infection in the pig;
wherein the administration of the one dose of the immunogenic composition to the pig in the vaccination method lessens the severity of clinical signs or clinical symptoms resulting from PCV3 infection in the pig;
wherein the protein according to any one of the preceding clauses is the antigenic component in the one dose of the immunogenic composition in the vaccination method that lessens the severity of clinical signs or clinical symptoms resulting from PCV3 infection in the pig;
preferably wherein the protein is in an amount of at least 2 μg in the one dose of the immunogenic composition;
wherein the protein is the antigenic component in the vaccination method that lessens the severity of clinical signs or clinical symptoms resulting from PCV3 infection in the pig.

188. An immunogenic composition according to any one of the preceding clauses for lessening the severity of clinical signs or clinical symptoms resulting from PCV3 infection in a pig;
wherein only one dose of the immunogenic composition is administered to the pig in a vaccination method to lessen the severity of clinical signs or clinical symptoms resulting from PCV3 infection in the pig;
wherein the administration of the one dose of the immunogenic composition to the pig in the vaccination method lessens the severity of clinical signs or clinical symptoms resulting from PCV3 infection in the pig;
wherein the protein according to any one of the preceding clauses is the antigenic component in the one dose of the immunogenic composition in the vaccination method that lessens the severity of clinical signs or clinical symptoms resulting from PCV3 infection in the pig;
preferably wherein the protein is in an amount of at least 2 μg in the one dose of the immunogenic composition;
wherein the protein is the antigenic component in the vaccination method that lessens the severity of clinical signs or clinical symptoms resulting from PCV3 infection in the pig.

189. An immunogenic composition according to any one of the preceding clauses for lessening the severity of clinical signs or clinical symptoms resulting from PCV3 infection in a pig;
wherein two doses of the immunogenic composition is administered to the pig in a vaccination method to lessen the severity of clinical signs or clinical symptoms resulting from PCV3 infection in the pig;
wherein the administration of the two doses of the immunogenic composition to the pig in the vaccination method lessens the severity of clinical signs or clinical symptoms resulting from PCV3 infection in the pig;
wherein the protein according to any one of the preceding clauses is the antigenic component in the two doses of the immunogenic composition in the vaccination method that lessens the severity of clinical signs or clinical symptoms resulting from PCV3 infection in the pig;
preferably wherein the protein is in an amount of at least 2 μg in the one dose of the immunogenic composition;
wherein the protein is the antigenic component in the vaccination method that lessens the severity of clinical signs or clinical symptoms resulting from PCV3 infection in the pig.

190. An immunogenic composition according to any one of the preceding clauses for lessening the severity of clinical signs or clinical symptoms resulting from PCV3 infection in a pig;
wherein only two doses of the immunogenic composition is administered to the pig in a vaccination method to lessen the severity of clinical signs or clinical symptoms resulting from PCV3 infection in the pig;
wherein the administration of the two doses of the immunogenic composition to the pig in the vaccination method lessens the severity of clinical signs or clinical symptoms resulting from PCV3 infection in the pig;
wherein the protein according to any one of the preceding clauses is the antigenic component in the two doses of the immunogenic composition in the vaccination method that lessens the severity of clinical signs or clinical symptoms resulting from PCV3 infection in the pig;
preferably wherein the protein is in an amount of at least 2 μg in the one dose of the immunogenic composition;
wherein the protein is the antigenic component in the vaccination method that lessens the severity of clinical signs or clinical symptoms resulting from PCV3 infection in the pig.

191. The use according to any one of the preceding clauses wherein said clinical signs or symptoms are selected from the group consisting of reduction of average daily weight gain and mortality.

192. The use according to any one of the preceding clauses wherein said clinical signs or symptoms are selected from the group consisting of gross lesions, histological lesions, replication of PCV3 in a tissue, and PCV3 viremia.

193. The use according to any one of the preceding clauses wherein said clinical signs or symptoms are selected from the group consisting of development or production of a mummified fetus.

194. The use according to any one of the preceding clauses wherein said clinical signs or symptoms is or include expelling of a mummified, stillborn and/or weak fetus.

### Clause Set D:

Clause Set D—The present invention will now be described by way of the following set of numbered clauses (Clause Set D). The disclosure in this set of clauses is equally applicable to the present invention. Likewise the disclosure in this set of clauses is equally applicable to each of the other set of clauses.

1. A porcine circovirus type 3 (PCV3) antigenic protein wherein said protein is a functional antigenic variant of PCV3 ORF2 protein.

2. A protein according to clause 1 wherein said PCV3 ORF2 protein is a protein encoded by SEQ ID No. 1.

3. A protein according to clause 1 or clause 2 wherein said protein comprises substitutions and/or extensions of PCV3 ORF2.

4. A protein according to any one of the preceding clauses wherein said protein is a functional antigenic variant of the protein encoded by SEQ ID No. 1.

5. A protein according to any one of the preceding clauses wherein said functional antigenic variant is capable of a higher yield of virus-like particles (VLPs) than the protein encoded by SEQ ID No. 1.

6. A protein according to any one of the preceding clauses wherein said functional antigenic variant is capable of a higher yield of VLPs than the protein encoded by SEQ ID No. 1 as determinable by Western blot analysis.

7. A protein according to any one of the preceding clauses wherein said functional antigenic variant has fewer positive charged amino acid residues than the protein encoded by SEQ ID No. 1.

8. A protein according to any one of the preceding clauses wherein said functional antigenic variant has one or more substitutions in the FG loop of the protein encoded by SEQ ID No. 1.

9. A protein according to any one of the preceding clauses wherein said functional antigenic variant has one or more substitutions in the FG loop of the protein encoded by SEQ ID No. 1, wherein those substitutions comprise substitutions of one or more of the S residue and/or the K residues and/or the H residue of the motif SKKKH of the FG loop of the protein encoded by SEQ ID No. 1.

10. A protein according to any one of the preceding clauses wherein said functional antigenic variant has one or more substitutions in the FG loop of the protein encoded by SEQ ID No. 1, wherein those substitutions comprise substitutions of one or more of the S residue and/or the K residues of the motif SKKKH of the FG loop of the protein encoded by SEQ ID No. 1.

11. A protein according to any one of the preceding clauses wherein said functional antigenic variant has one or more substitutions in the FG loop of the protein encoded by SEQ ID No. 1, wherein those substitutions comprise substitutions of the S residue or H residue and all of the K residues of the motif SKKKH of the FG loop of the protein encoded by SEQ ID No. 1.

12. A protein according to any one of the preceding clauses wherein said functional antigenic variant has one or more substitutions in the FG loop of the protein encoded by SEQ ID No. 1, wherein those substitutions comprise a substitution of at least S and/or H and any K of the motif SKKKH of the FG loop of the protein encoded by SEQ ID No. 1 with Q or P or F or S.

13. A protein according to any one of the preceding clauses wherein said functional antigenic variant has one or more substitutions in the FG loop of the protein encoded by SEQ ID No. 1, wherein those substitutions comprise substitution of the motif SKKK within the motif SKKKH of the FG loop of the protein encoded by SEQ ID No. 1 with QPFS or substitution of the motif KKKH within the motif SKKKH of the FG loop of the protein encoded by SEQ ID No. 1 with QPFS.

14. A protein according to any one of the preceding clauses wherein said functional antigenic variant is encodable by all or part of SEQ ID No. 1, 2, 5, 6 or 7.

15. A protein according to any one of the preceding clauses wherein said functional antigenic variant is encoded by all or part of SEQ ID No. 1, 2, 5, 6 or 7.

16. A protein according to any one of the preceding clauses wherein said functional antigenic variant has a C terminal end that extends beyond the terminal SVL sequence of the protein encoded by SEQ ID No. 1, preferably wherein said extension is all or includes a sequence from a circoviridae virus, and preferably wherein at least a part of said extension replaces the terminal SVL sequence of the protein encoded by SEQ ID No. 1.

17. A protein according to any one of the preceding clauses wherein said functional antigenic variant has a C terminal end that extends beyond the terminal SVL sequence of the protein encoded by SEQ ID No. 1; and wherein said extension is from 1 to 100 amino acids long.

18. A protein according to any one of the preceding clauses wherein said functional antigenic variant has a C terminal end that extends beyond the terminal SVL sequence of the protein encoded by SEQ ID No. 1; and wherein said extension is from 1 to 50 amino acids long.

19. A protein according to any one of the preceding clauses wherein said functional antigenic variant has a C terminal end that extends beyond the terminal SVL sequence of the protein encoded by SEQ ID No. 1; and wherein said extension is from 1 to 30 amino acids long.

20. A protein according to any one of the preceding clauses wherein said functional antigenic variant has a C terminal end that extends beyond the terminal SVL sequence of the protein encoded by SEQ ID No. 1; wherein said extension is from 1 to 30 amino acids long; and wherein said extension all of part of the sequence comprises VKININLTPPVATSRVPSRALPLRFGCGHR.

21. A protein according to any one of the preceding clauses wherein said functional antigenic variant has a C terminal end that extends beyond the terminal SVL sequence of the protein encoded by SEQ ID No. 1; wherein said extension is from 1 to 30 amino acids long; and wherein said extension comprises all of the sequence VKININLTPPVATSRVPSRALPLRFGCGHR.

22. A protein according to any one of the preceding clauses wherein said functional antigenic variant is encodable by all or part of SEQ ID No. 1, 2, 5, 6 or 7.

23. A protein according to any one of the preceding clauses wherein said functional antigenic variant is encoded by all or part of SEQ ID No. 1, 2, 5, 6 or 7.

24. A protein according to any one of the preceding clauses wherein said protein is recombinant protein having been prepared by recombinant DNA techniques.

25. A protein according to any one of the preceding clauses wherein said protein is baculovirus expressed protein.

26. A protein according to any one of the preceding clauses wherein said PCV3 is selected from the group consisting of PCV3a and PCV3b.

27. A protein according to any one of the preceding clauses wherein said PCV3 is any phylogenetic clade of PCV3 or selected from the group consisting PCV3a1, PCV3b1, PCV3b2 and PCV3c.

28. A protein according to any one of the preceding clauses wherein said PCV3 ORF2 is from group a1, b1 or b2 (using the subtyping designation of Fux et al., “Full genome characterization of porcine circovirus type 3 isolates reveals the existence of two distinct groups of virus strains,” Virology Journal (2018) 15:25, DOI 10.1186/s12985-018-0929-3 (incorporated herein by reference); see, e.g., Table 4).

29. A protein according to any one of the preceding clauses wherein said PCV3 ORF2 protein comprises or consists of an amino acid sequence encoded by a polynucleotide sequence having at least 90%, or at least 91%, or at least 92%, or at least 93%, or at least 94%, or at least 95%, or at least 96%, or at least 97%, or at least 98%, or at least 99%, or 100% sequence identity with SEQ ID NO: 1 or sequence homology with SEQ ID NO:1.

30. A protein according to any one of the preceding clauses wherein said variant protein comprises or consists of an amino acid sequence encoded by a polynucleotide sequence having at least 90%, or at least 91%, or at least 92%, or at least 93%, or at least 94%, or at least 95%, or at least 96%, or at least 97%, or at least 98%, or at least 99%, or 100% sequence identity with SEQ ID NO:1 or sequence homology with SEQ ID NO:6.

31. A protein according to any one of the preceding clauses wherein said variant protein comprises or consists of an amino acid sequence encoded by a polynucleotide sequence having at least 90%, or at least 91%, or at least 92%, or at least 93%, or at least 94%, or at least 95%, or at least 96%, or at least 97%, or at least 98%, or at least 99%, or 100% sequence identity with SEQ ID NO:1 or sequence homology with SEQ ID NO:7.

32. A protein according to any one of the preceding clauses wherein said PCV3 ORF2 protein comprises or consists of an amino acid sequence having at least 90%, or at least 91%, or at least 92%, or at least 93%, or at least 94%, or at least 95%, or at least 96%, or at least 97%, or at least 98%, or at least 99%, or 100% sequence identity with the sequence of SEQ ID NO: 3, 4, 8, 9 or 10 and/or wherein the protein is a recombinant protein.

33 A protein according to any one of the preceding clauses wherein said variant protein comprises or consists of an amino acid sequence having at least 90%, or at least 91%, or at least 92%, or at least 93%, or at least 94%, or at least 95%, or at least 96%, or at least 97%, or at least 98%, or at least 99%, or 100% sequence identity with the sequence of SEQ ID NO: 3, 4, 8, 9 or 10 and/or wherein the protein is a recombinant protein; or a protein according to any one of the preceding clauses wherein said variant protein comprises or consists of an amino acid sequence having at least 90%, or at least 91%, or at least 92%, or at least 93%, or at least 94%, or at least 95%, or at least 96%, or at least 97%, or at least 98%, or at least 99%, or 100% sequence identity with the sequence of SEQ ID NO: 3, 4, 8, 9 or 10, and/or wherein the protein is a recombinant protein, and wherein said protein has one or more substitutions in the FG loop.

34. A protein according to any one of the preceding clauses wherein said variant protein comprises or consists of an amino acid sequence having at least 90%, or at least 91%, or at least 92%, or at least 93%, or at least 94%, or at least 95%, or at least 96%, or at least 97%, or at least 98%, or at least 99%, or 100% sequence identity with the sequence of SEQ ID NO: 3, 4, 8, 9 or 10 and/or wherein the protein is a recombinant protein; or a protein according to any one of the preceding clauses wherein said variant protein comprises or consists of an amino acid sequence having at least 90%, or at least 91%, or at least 92%, or at least 93%, or at least 94%, or at least 95%, or at least 96%, or at least 97%, or at least 98%, or at least 99%, or 100% sequence identity with the sequence of SEQ ID NO: 3, 4, 8, 9 or 10, and/or wherein the protein is a recombinant protein, and wherein said protein has a C terminal end that extends beyond the terminal SVL sequence of the protein encoded by SEQ ID No. 1.

35. A protein according to any one of the preceding clauses wherein said protein is a recombinant protein from expression thereof by an expression vector, comprising a polynucleotide sequence that encodes the protein.

36. A protein according to any one of the preceding clauses wherein said protein is a recombinant protein from expression thereof by a baculovirus expression vector, comprising a polynucleotide sequence that encodes the protein.

37. A nucleotide sequence encoding the protein according to any of the preceding clauses.

38. A vector comprising the nucleotide sequence of any of the preceding clauses.

39. A recombinant vector comprising the nucleotide sequence of any of the preceding clauses.

40. An expression host transformed or transfected with the nucleotide sequence of any of the preceding clauses.

41. A baculovirus expression host transformed or transfected with the nucleotide sequence of any of the preceding clauses.

42. A method of preparing a protein according to any one of the preceding clauses comprising expressing a nucleotide sequence according to any of the preceding clauses.

43. A method of preparing a protein according to any one of the preceding clauses comprising expressing a vector according to any of the preceding clauses.

44. A method of preparing a protein according to any one of the preceding clauses comprising expressing a recombinant vector according to any of the preceding clauses.

45. A method of preparing a protein according to any one of the preceding clauses comprising culturing the expression host according to any of the preceding clauses to cause expression of the protein.

46. A method of preparing a protein according to any one of the preceding clauses comprising transfecting an expression host with the nucleotide sequence of vector according to any one of the preceding clauses and culturing the expression host to cause expression of the protein.

47. A method of preparing a protein according to any one of the preceding clauses comprising culturing the baculovirus expression host according to any of the preceding clauses to cause expression of the protein.

48. A method of preparing a protein according to any one of the preceding clauses comprising transfecting a baculovirus expression host with the nucleotide sequence of vector according to any one of the preceding clauses and culturing the baculovirus expression host to cause expression of the protein.

49. A method according to any one of the preceding clauses wherein an inactivating agent is used when sufficient levels of expressed protein have been achieved.

50. A method according to any one of the preceding clauses wherein an inactivating agent comprising binary ethyleneimine (BEI) is used when sufficient levels of expressed protein have been achieved.

51. A method of preparing a protein according to any one of the preceding clauses comprising transfecting a baculovirus expression host with the nucleotide sequence of vector according to any one of the preceding clauses and culturing the baculovirus expression host in a medium to cause expression of the protein; wherein the medium post expression of the protein comprises (i) said protein, (ii) at least a portion of baculovirus that expressed said protein, (iii) a portion of cell culture of cells that were infected or transfected with recombinant baculovirus that expressed said protein.

52. A method of preparing a protein according to any one of the preceding clauses comprising transfecting a baculovirus expression host with the nucleotide sequence of vector according to any one of the preceding clauses and culturing the baculovirus expression host in a medium to cause expression of the protein; wherein the medium post expression of the protein comprises (i) said protein, (ii) at least a portion of baculovirus that expressed said protein, (iii) a portion of cell culture of cells that were infected or transfected with recombinant baculovirus that expressed said protein; and wherein about 90% of the components (i) to (iii) have a size smaller than 1 μm.

53. A method of preparing a protein according to any one of the preceding clauses comprising transfecting a baculovirus expression host with the nucleotide sequence of vector according to any one of the preceding clauses and culturing the baculovirus expression host in a medium to cause expression of the protein; wherein the medium post expression of the protein comprises (i) said protein, (ii) at least a portion of baculovirus that expressed said protein, (iii) a portion of cell culture of cells that were infected or transfected with recombinant baculovirus that expressed said protein; and wherein about 90% of the components (i) to (iii) have a size smaller than 1 μm and the pH of said composition is adjusted to about 6.5 to 7.5.

54. A method of preparing a protein according to any one of the preceding clauses comprising producing the protein by a baculovirus expression system in cultured insect cells.

55. A method of preparing a protein according to any one of the preceding clauses comprising producing the protein by a baculovirus expression system in cultured insect cells; and wherein the method includes the step of inactivating the baculovirus.

56. A method of preparing a protein according to any one of the preceding clauses comprising producing the protein by a baculovirus expression system in cultured insect cells; and wherein the method includes the step of inactivating the baculovirus; and wherein inactivating step comprises heat treatment or use of a virus inactivating agent.

57. A method of preparing a protein according to any one of the preceding clauses comprising producing the protein by a baculovirus expression system in cultured insect cells; and wherein the method includes the step of inactivating the baculovirus; and wherein inactivating step comprises heat treatment or use of a virus inactivating agent; and wherein the virus inactivating agent comprises an aziridine compound.

58. A method of preparing a protein according to any one of the preceding clauses comprising producing the protein by a baculovirus expression system in cultured insect cells; and wherein the method includes the step of inactivating the baculovirus; and wherein inactivating step comprises heat treatment or use of a virus inactivating agent; and wherein the virus inactivating agent comprises an aziridine compound; wherein the aziridine compound comprises BEI.

59. A protein obtainable by the method according to any one of the preceding clauses.

60. A composition comprising the protein obtainable by the method according to any one of the preceding clauses.

61. A composition obtainable by the method according to any one of the preceding clauses.

62. A composition comprising a protein according to any one of the preceding clauses and a carrier, diluent or excipient.

63. A composition comprising a protein according to any one of the preceding clauses and a veterinary-acceptable carrier, diluent or excipient.

64. A composition according to any one of the preceding clauses wherein the protein is present in an amount of 0.2 to about 400 μg/ml, or 2 to about 400 μg/ml, or 4 to about 400 μg/ml, or 8 to about 400 μg/ml, or about 0.3 to about 200 μg/ml, or 2 to about 200 μg/ml, or 4 to about 200 μg/ml, or 8 to about 200 μg/ml, or about 0.35 to about 100 μg/ml, or 2 to about 100 μg/ml, or 4 to about 100 μg/ml, or 8 to about 100 μg/ml, or about 0.4 to about 50 μg/ml, or about 0.45 to about 30 g/ml, or about 0.6 to about 15 μg/ml, or about 0.75 to about 8 μg/ml, or about 1.0 to about 6 μg/ml, or about 1.3 to about 3.0 μg/ml, or about 1.4 to about 2.5 μg/ml, or about 1.5 to about 2.0 μg/ml, or about 1.6 μg/ml.

65. A composition comprising a protein according to any one of the preceding clauses wherein the composition comprises any one or more of a solvent, a dispersion media, a coating, a stabilizing agent, a diluent, a preservative, an anti-microbial agent, an antifungal agent, an isotonic agent, an adsorption delaying agent, an adjuvant, cell culture supernatant, a stabilizing agent, a viral vector, an expression vector, and/or an immunomodulatory agent.

66. A composition according to any one of the preceding clauses wherein the carrier, diluent or excipient is any one or more of an adjuvant, immunomodulatory agent, cell culture supernatant, viral or expression vector or any combination thereof.

67. A composition according to any one of the preceding clauses wherein the carrier, diluent or excipient comprises an adjuvant.

68. A composition according to any one of the preceding clauses wherein the carrier, diluent or excipient comprises an adjuvant; wherein the adjuvant comprises one or more of a polymer of acrylic or methacrylic acid; copolymer of maleic anhydride and alkenyl derivative; a polymer of acrylic or methacrylic acid which is cross-linked; a polymer of acrylic or methacrylic acid which is cross-linked with a polyalkenyl ether of sugar or polyalcohol; a carbomer; an acrylic polymer cross-linked with a polyhydroxylated compound having at least 3 and not more than 8 hydroxyl groups with hydrogen atoms of at least three hydroxyls optionally or being replaced by unsaturated aliphatic radicals having at least 2 carbon atoms with said radicals containing from 2 to 4 carbon atoms such as vinyls, allyls and other ethylenically unsaturated groups and the unsaturated radicals may themselves contain other substituents, such as methyl; a carbopol; Carbopol 974P; Carbopol 934P; Carbopol 971P; aluminum hydroxide; aluminum phosphate; a saponin; Quil A; QS-21; GPI-0100; a water-in-oil emulsion; an oil-in-water emulsion; a water-in-oil-in-water emulsion; an emulsion based on light liquid paraffin oil or European Pharmacopea type adjuvant; an isoprenoid oil; squalane; squalene oil resulting from oligomerization of alkenes or isobutene or decene; (an) ester(s) of acid(s) or of alcohol(s) containing a linear alkyl group; plant oil(s); ethyl oleate; propylene glycol di-(caprylate/caprate); glyceryl tri-(caprylate/caprate); propylene glycol dioleate; (an) ester(s) of branched fatty acid(s) or alcohol(s); isostearic acid ester(s); nonionic surfactant(s); (an) ester(s) of sorbitan or of mannide or of glycol or of polyglycerol or of propylene glycol or of oleic, or isostearic acid or of ricinoleic acid or of hydroxystearic acid, optionally ethoxylated, anhydromannitol oleate; polyoxypropylene-polyoxyethylene copolymer blocks, a Pluronic product, RIBI adjuvant system; Block co-polymer; SAF-M; monophosphoryl lipid A; Avridine lipid-amine adjuvant; heat-labile enterotoxin from E. coli (recombinant or otherwise); cholera toxin; IMS 1314, or muramyl dipeptide.

69. A composition according to any one of the preceding clauses wherein the carrier, diluent or excipient comprises an adjuvant; wherein the adjuvant comprises Carbopol or Carbopol 971.

70. A composition according to any one of the preceding clauses wherein the carrier, diluent or excipient comprises an adjuvant; wherein the adjuvant is present in an amount from about 50 μg to about 2000 of the composition; or wherein adjuvant is present in an amount about 250 μg/ml dose of the composition, or wherein the adjuvant is present in an amount of about 100 μg to about 10 mg of the composition; or wherein the adjuvant is present in an amount of about 500 μg to about 5 mg of the composition; the adjuvant is present in an amount of about 750 μg to about 2.5 mg of the composition; or the adjuvant is present in an amount of about 1 mg of the composition.

71. A composition according to any one of the preceding clauses wherein the composition comprises an immunomodulatory agent.

72. A composition according to any one of the preceding clauses wherein the composition comprises an immunomodulatory agent; and wherein the immunomodulatory agent is any one or more of interleukin(s), interferon(s), or other cytokine(s).

73. A composition according to any one of the preceding clauses wherein the composition comprises an antibiotic(s).

74. A composition according to any one of the preceding clauses wherein the composition comprises an antibiotic(s); wherein the antibiotic(s) comprise Gentamicin.

75. A composition according to any one of the preceding clauses wherein the composition comprises an antibiotic(s); and wherein the composition comprises from about 1 μg/ml to about 60 μg/ml of antibiotic(s).

76. A composition according to any one of the preceding clauses wherein the composition comprises an antibiotic(s); and wherein the composition comprises from about 1 μg/ml to less than about 30 μg/ml of antibiotic(s).

77. A composition according to any one of the preceding clauses wherein the composition comprises an additional antigen.

78. A composition according to any one of the preceding clauses wherein the composition comprises an additional antigen; wherein said additional antigen is not a PCV3 ORF2 antigen.

79. A composition according to any one of the preceding clauses wherein the composition comprises an additional antigen; wherein said additional antigen is not a PCV3 antigen.

80. A composition according to any one of the preceding clauses wherein the composition comprises an additional antigen of an additional porcine pathogen.

81. A composition according to any one of the preceding clauses wherein the composition further comprises an antigen of an additional porcine pathogen, wherein said pathogen is any one or more of PCV2, PRRSV (porcine respiratory and reproductive syndrome virus) antigen, a Mycoplasma hyopneumoniae bacterin antigen, a Mycoplasma hyopneumoniae supernatant antigen, an Aujeszky's disease or pseudorabies antigen, a swine influenza antigen, a swine fever antigen (classical or African or combination thereof), an Actinobacillus pleuropneumoniae antigen, an Escherichia coli antigen, a porcine parvovirus (PPV) antigen or a Pasteurella multocida antigen.

82. A composition according to any one of the preceding clauses wherein the composition further comprises an antigen of an additional porcine pathogen, wherein said composition further comprises one or more of an antigen of PCV2, an antigen of a PRRSV and an antigen of a PPV.

83. A composition according to any one of the preceding clauses wherein the composition further comprises an antigen of PCV2.

84. A composition according to any one of the preceding clauses wherein the composition further comprises an antigen of PCV2; wherein PCV2 antigen is PCV2 ORF2 protein.

85. A composition according to any one of the preceding clauses wherein the composition further comprises an antigen of PCV2; wherein PCV2 antigen is recombinant PCV2 ORF2 protein.

86. A composition according to any one of the preceding clauses wherein the composition further comprises an antigen of PCV2; wherein PCV2 antigen is recombinant baculovirus expressed PCV2 ORF2 protein.

87. A composition according to any one of the preceding clauses wherein the composition is in a dosage form.

88. A composition according to any one of the preceding clauses wherein the composition is formulated and/or packaged for a single dose or one shot administration.

89. A composition according to any one of the preceding clauses wherein the composition is formulated and/or packaged for a multi-dose regimen.

90. A composition according to any one of the preceding clauses wherein the composition is formulated and/or packaged for a two-dose regimen.

91. A composition according to any one of the preceding clauses wherein the composition is in a dosage form; and wherein said dosage form is delivered from a container containing a larger amount of said composition and wherein a dosage form of said composition is capable of being delivered from said container.

92. A composition according to any one of the preceding clauses wherein the composition is in a dosage form; and wherein said dosage form is delivered from a container containing a larger amount of said composition and wherein a dosage form of said composition is capable of being delivered from said container; and wherein said container contains at least 10 doses of said composition.

93. A composition according to any one of the preceding clauses wherein the composition is in a dosage form; and wherein said dosage form is delivered from a container containing a larger amount of said composition and wherein a dosage form of said composition is capable of being delivered from said container; and wherein said container contains at least 50 doses of said composition.

94. A composition according to any one of the preceding clauses wherein the composition is in a dosage form; and wherein said dosage form is delivered from a container containing a larger amount of said composition and wherein a dosage form of said composition is capable of being delivered from said container; and wherein said container contains at least 100 doses of said composition.

95. A composition according to any one of the preceding clauses wherein the composition is in a dosage form; and wherein said dosage form is delivered from a container containing a larger amount of said composition and wherein a dosage form of said composition is capable of being delivered from said container; and wherein said container contains at least 200 doses of said composition.

96. A composition according to any one of the preceding clauses wherein the composition is in a dosage form; and wherein said dosage form is delivered from a container containing a larger amount of said composition and wherein a dosage form of said composition is capable of being delivered from said container; and wherein said container contains at least 250 doses of said composition.

97. A composition according to any one of the preceding clauses wherein the composition comprises an antigen of PCV2; wherein PCV2 antigen is recombinant baculovirus expressed PCV2 ORF2 protein; and wherein either the protein or combined total amount of the PCV3 ORF2 protein and PCV2 ORF protein are present in an amount of about 0.2 to about 400 μg/dose, or 2 to about 400 μg/dose, or 4 to about 400 μg/dose, or 8 to about 400 μg/dose, or about 0.3 to about 200 μg/dose, or 2 to about 200 μg/dose, or 4 to about 200 μg/dose, or 8 to about 200 μg/dose, or about 0.35 to about 100 μg/dose, or 2 to about 100 μg/dose, or 4 to about 100 μg/dose, or 8 to about 100 μg/dose, or about 0.4 to about 50 μg/dose, or about 0.45 to about 30 μg/dose, or about 0.6 to about 15 μg/dose, or about 0.75 to about 8 μg/dose, or about 1.0 to about 6 μg/dose, or about 1.3 to about 3.0 μg/dose, or about 1.4 to about 2.5 μg/dose, or about 1.5 to about 2.0 μg/dose, or about 1.6 μg/dose.

98. A composition according to any one of the preceding clauses wherein the composition comprises a salt.

99. A composition according to any one of the preceding clauses wherein the composition comprises an inactivated viral vector and/or cell culture supernate.

100. A composition according to any one of the preceding clauses wherein the composition comprises an inactivated viral vector and cell culture supernate.

101. A composition according to any one of the preceding clauses wherein the composition comprises (i) the protein, (ii) at least a portion of baculovirus that expressed said protein, (iii) a portion of cell culture of cells that were infected or transfected with recombinant baculovirus that expressed said protein, (iv) inactivating agent or inactivating agent comprising binary ethyleneimine (BEI), (v) sodium thiosulfate or sodium thiosulfate in equivalent amounts to inactivating agent or BEI; (vi) adjuvant or adjuvant comprising Carbopol or Carbopol 971, and (vii) phosphate salt in a physiologically acceptable concentration.

102. A composition according to any one of the preceding clauses wherein the composition comprises (i) the protein, (ii) at least a portion of baculovirus that expressed said protein, (iii) a portion of cell culture of cells that were infected or transfected with recombinant baculovirus that expressed said protein, (iv) inactivating agent or inactivating agent comprising binary ethyleneimine (BEI), (v) sodium thiosulfate or sodium thiosulfate in equivalent amounts to inactivating agent or BEI; (vi) adjuvant or adjuvant comprising Carbopol or Carbopol 971, and (vii) phosphate salt in a physiologically acceptable concentration; and wherein the BEI is from the cell culture having been treated with about 2 to 8 or about 5 mM BEI to inactivate the baculovirus and/or the composition contains about 2 to 8 or about 5 mM BEI and/or the composition contains about 1 mg of the Carbopol or Carbopol 971.

103. A composition according to any one of the preceding clauses wherein the composition is an immunogenic composition comprising a protein according to any one of the preceding clauses and a carrier, diluent or excipient.

104. A composition according to any one of the preceding clauses wherein the composition is an immunogenic composition comprising a protein according to any one of the preceding clauses and a carrier, diluent or excipient; and an additional antigen according to any one of the preceding clauses.

105. A process of making the composition according to any one of the preceding clauses wherein the protein according to any one of the preceding clauses is admixed with the carrier, diluent or excipient.

106. A process of making the composition according to any one of the preceding clauses wherein the protein according to any one of the preceding clauses is admixed with the carrier, diluent or excipient; and the additional antigen.

107. A protein according to any one of the preceding clauses or a composition according to any one of the preceding clauses for use as a medicament.

108. A protein according to any one of the preceding clauses or a composition according to any one of the preceding clauses for use as a vaccine.

109. A protein according to any one of the preceding clauses or a composition according to any one of the preceding clauses for use in a method for eliciting an immune response or an immunological response or a protective immune or immunological response against PCV3 in an animal.

110. A protein according to any one of the preceding clauses or a composition according to any one of the preceding clauses for use in a method for eliciting an immune response or an immunological response or a protective immune or immunological response against PCV3 in swine.

111. A protein according to any one of the preceding clauses or a composition according to any one of the preceding clauses for use in a method for eliciting an immune response or an immunological response or a protective immune or immunological response against PCV3 in pigs.

112. A protein according to any one of the preceding clauses or a composition according to any one of the preceding clauses for use in a method for eliciting an immune response or an immunological response or a protective immune or immunological response against PCV3 in piglets.

113. A protein according to any one of the preceding clauses or a composition according to any one of the preceding clauses for use in a method for eliciting an immune response or an immunological response or a protective immune or immunological response against PCV3 in piglets; wherein the piglets are to be suckled by sows to which the protein according to any one of the preceding clauses or a composition according to any one of the preceding clauses has been administered.

114. A protein according to any one of the preceding clauses or a composition according to any one of the preceding clauses for use in a method for eliciting an immune response or an immunological response or a protective immune or immunological response against PCV3 in sows.

115. A protein according to any one of the preceding clauses or a composition according to any one of the preceding clauses for use in a method for eliciting an immune response or an immunological response or a protective immune or immunological response against PCV3 in pregnant sows, gilts or pre-breeding gilts.

116. A protein according to any one of the preceding clauses or a composition according to any one of the preceding clauses for use in inducing an immune response against PCV3 in animals.

117. A protein according to any one of the preceding clauses or a composition according to any one of the preceding clauses for use in inducing an immune response against PCV3 in swine.

118. A protein according to any one of the preceding clauses or a composition according to any one of the preceding clauses for use in inducing an immune response against PCV3 in pigs.

119. A protein according to any one of the preceding clauses or a composition according to any one of the preceding clauses for use in inducing an immune response against PCV3 in piglets.

120. A protein according to any one of the preceding clauses or a composition according to any one of the preceding clauses for use in inducing an immune response against PCV3 in piglets; wherein the piglets are to be suckled by sows to which the protein according to any one of the preceding clauses or a composition according to any one of the preceding clauses has been administered.

121. A protein according to any one of the preceding clauses or a composition according to any one of the preceding clauses for use in inducing an immune response against PCV3 in sows.

122. A protein according to any one of the preceding clauses or a composition according to any one of the preceding clauses for use in inducing an immune response against PCV3 in pregnant sows, gilts or pre-breeding gilts.

123. A protein according to any one of the preceding clauses or a composition according to any one of the preceding clauses for use in a method of reducing or preventing the clinical signs or clinical symptoms or disease caused by an infection with PCV3 in an animal or for use in a method of treating or preventing an infection with PCV3 in an animal.

124. A protein according to any one of the preceding clauses or a composition according to any one of the preceding clauses for use in a method of reducing or preventing the clinical signs or clinical symptoms or disease caused by an infection with PCV3 in an animal or for use in a method of treating or preventing an infection with PCV3 in an animal; wherein said animal is swine.

125. A protein according to any one of the preceding clauses or a composition according to any one of the preceding clauses for use in a method of reducing or preventing the clinical signs or clinical symptoms or disease caused by an infection with PCV3 in an animal or for use in a method of treating or preventing an infection with PCV3 in an animal; wherein said animal is a pig.

126. A protein according to any one of the preceding clauses or a composition according to any one of the preceding clauses for use in a method of reducing or preventing the clinical signs or clinical symptoms or disease caused by an infection with PCV3 in an animal or for use in a method of treating or preventing an infection with PCV3 in an animal; wherein said animal is a piglet.

127. A protein according to any one of the preceding clauses or a composition according to any one of the preceding clauses for use in a method of reducing or preventing the clinical signs or clinical symptoms or disease caused by an infection with PCV3 in an animal or for use in a method of treating or preventing an infection with PCV3 in an animal; wherein said animal is a piglet; and wherein the piglet is to be suckled by a sow to which the protein according to any one of the preceding clauses or a composition according to any one of the preceding clauses has been administered.

128. A protein according to any one of the preceding clauses or a composition according to any one of the preceding clauses for use in a method of reducing or preventing the clinical signs or disease caused by an infection with PCV3 in an animal or for use in a method of treating or preventing an infection with PCV3 in an animal; wherein said animal is a sow.

129. A protein according to any one of the preceding clauses or a composition according to any one of the preceding clauses for use in a method of reducing or preventing the clinical signs or clinical symptoms or disease caused by an infection with PCV3 in an animal or for use in a method of treating or preventing an infection with PCV3 in an animal; wherein said animal is a pregnant sow, gilt or pre-breeding gilt.

130. A protein according to any one of the preceding clauses or a composition according to any one of the preceding clauses for use in immunizing an animal against PCV3.

131. A protein according to any one of the preceding clauses or a composition according to any one of the preceding clauses for use in immunizing an animal against PCV3; wherein said animal is swine.

132. A protein according to any one of the preceding clauses or a composition according to any one of the preceding clauses for use in immunizing an animal against PCV3; wherein said animal is a pig.

133. A protein according to any one of the preceding clauses or a composition according to any one of the preceding clauses for use in immunizing an animal against PCV3; wherein said animal is a piglet.

134. A protein according to any one of the preceding clauses or a composition according to any one of the preceding clauses for use in immunizing an animal against PCV3; wherein said animal is a piglet; and wherein the piglet is to be suckled by a sow to which the protein according to any one of the preceding clauses or a composition according to any one of the preceding clauses has been administered.

135. A protein according to any one of the preceding clauses or a composition according to any one of the preceding clauses for use in immunizing an animal against PCV3; wherein said animal is a sow.

136. A protein according to any one of the preceding clauses or a composition according to any one of the preceding clauses for use in immunizing an animal against PCV3; wherein said animal is a pregnant sow, gilt or pre-breeding gilt.

137. A protein according to any one of the preceding clauses or a composition according to any one of the preceding clauses for use in a method of reducing or eliminating or abrogating PCV3 viral expression in an animal.

138. A protein according to any one of the preceding clauses or a composition according to any one of the preceding clauses for use in a method of reducing or eliminating or abrogating PCV3 viral expression in an animal; wherein said animal is swine.

139. A protein according to any one of the preceding clauses or a composition according to any one of the preceding clauses for use in a method of reducing or eliminating or abrogating PCV3 viral expression in an animal; wherein said animal is a pig.

140. A protein according to any one of the preceding clauses or a composition according to any one of the preceding clauses for use in a method of reducing or eliminating or abrogating PCV3 viral expression in an animal; wherein said animal is a piglet.

141. A protein according to any one of the preceding clauses or a composition according to any one of the preceding clauses for use in a method of reducing or eliminating or abrogating PCV3 viral expression in an animal; wherein said animal is a piglet; and wherein the piglet is to be suckled by a sow to which the protein according to any one of the preceding clauses or a composition according to any one of the preceding clauses has been administered.

141. A protein according to any one of the preceding clauses or a composition according to any one of the preceding clauses for use in a method of reducing or eliminating or abrogating PCV3 viral expression in an animal; wherein said animal is a sow.

142. A protein according to any one of the preceding clauses or a composition according to any one of the preceding clauses for use in a method of reducing or eliminating or abrogating PCV3 viral expression in an animal; wherein said animal is a pregnant sow, gilt or pre-breeding gilt.

143. A protein according to any one of the preceding clauses or a composition according to any one of the preceding clauses for use in inducing the production of antibodies specific for PCV3 in an animal.

144. A protein according to any one of the preceding clauses or a composition according to any one of the preceding clauses for use in inducing the production of antibodies specific for PCV3 in an animal; wherein said animal is swine.

145. A protein according to any one of the preceding clauses or a composition according to any one of the preceding clauses for use in inducing the production of antibodies specific for PCV3 in an animal; wherein said animal is a pig.

146. A protein according to any one of the preceding clauses or a composition according to any one of the preceding clauses for use in inducing the production of antibodies specific for PCV3 in an animal; wherein said animal is a piglet.

147. A protein according to any one of the preceding clauses or a composition according to any one of the preceding clauses for use in inducing the production of antibodies specific for PCV3 in an animal; wherein said animal is a piglet; and wherein the piglet is to be suckled by a sow to which the protein according to any one of the preceding clauses or a composition according to any one of the preceding clauses has been administered.

148. A protein according to any one of the preceding clauses or a composition according to any one of the preceding clauses for use in inducing the production of antibodies specific for PCV3 in an animal; wherein said animal is a sow.

149. A protein according to any one of the preceding clauses or a composition according to any one of the preceding clauses for use in inducing the production of antibodies specific for PCV3 in an animal; wherein said animal is a pregnant sow, gilt or pre-breeding gilt.

150. A protein according to any one of the preceding clauses or a composition according to any one of the preceding clauses for the use according to any one of the preceding clauses wherein said protein according to any one of the preceding clauses or a composition according to any one of the preceding clauses is administered intramuscularly or intradermally to said animal.

151. A protein according to any one of the preceding clauses or a composition according to any one of the preceding clauses for the use according to any one of the preceding clauses wherein said protein according to any one of the preceding clauses or a composition according to any one of the preceding clauses is administered to said animal in conjunction with another antigen, preferably wherein the other pathogen is an antigen to a porcine pathogen.

152. A protein according to any one of the preceding clauses or a composition according to any one of the preceding clauses for the use according to any one of the preceding clauses wherein said protein according to any one of the preceding clauses or a composition according to any one of the preceding clauses is administered to said animal in conjunction with another antigen; wherein said other antigen is not a PCV3 ORF2 antigen, preferably wherein the other pathogen is an antigen to a porcine pathogen.

153. A protein according to any one of the preceding clauses or a composition according to any one of the preceding clauses for the use according to any one of the preceding clauses wherein said protein according to any one of the preceding clauses or a composition according to any one of the preceding clauses is administered to said animal in conjunction with another antigen; wherein said other antigen is not a PCV3 antigen, preferably wherein the other pathogen is an antigen to a porcine pathogen.

154. A protein according to any one of the preceding clauses or a composition according to any one of the preceding clauses for the use according to any one of the preceding clauses wherein said animal is a sow pregnant with a piglet.

155. A protein according to any one of the preceding clauses or a composition according to any one of the preceding clauses for the use according to any one of the preceding clauses wherein said animal is a sow pregnant with a piglet; and wherein the piglet is to be suckled by a sow to which the protein according to any one of the preceding clauses or a composition according to any one of the preceding clauses has been administered.

156. A protein according to any one of the preceding clauses or a composition according to any one of the preceding clauses for the use according to any one of the preceding clauses wherein said animal is a sow; and wherein said protein according to any one of the preceding clauses or a composition according to any one of the preceding clauses is administered twice to said sow.

157. A protein according to any one of the preceding clauses or a composition according to any one of the preceding clauses wherein said animal is a sow; and wherein said protein according to any one of the preceding clauses or a composition according to any one of the preceding clauses is only administered twice to said sow.

158. A protein according to any one of the preceding clauses or a composition according to any one of the preceding clauses for the use according to any one of the preceding clauses wherein said animal is a piglet; and wherein the protein according to any one of the preceding clauses or a composition according to any one of the preceding clauses is administered once to said piglet.

159. A protein according to any one of the preceding clauses or a composition according to any one of the preceding clauses for the use according to any one of the preceding clauses wherein said animal is a piglet; and wherein the protein according to any one of the preceding clauses or a composition according to any one of the preceding clauses is only administered once to said piglet.

160. A protein according to any one of the preceding clauses or a composition according to any one of the preceding clauses for the use according to any one of the preceding clauses wherein said animal is a sow; and wherein the protein according to any one of the preceding clauses or a composition according to any one of the preceding clauses is administered twice to said sow; and wherein said use does not include the administration of any other PCV3 antigen to said animal before or during the administration of said protein according to any one of the preceding clauses or a composition according to any one of the preceding clauses.

161. A protein according to any one of the preceding clauses or a composition according to any one of the preceding clauses for the use according to any one of the preceding clauses wherein said animal is a sow; and wherein the protein according to any one of the preceding clauses or a composition according to any one of the preceding clauses is administered twice to said sow; and wherein said use does not include the administration of any other PCV3 antigen to said animal before or during the administration of said protein according to any one of the preceding clauses or a composition according to any one of the preceding clauses.

162. A protein according to any one of the preceding clauses or a composition according to any one of the preceding clauses for the use according to any one of the preceding clauses wherein said animal is a sow; and wherein the protein according to any one of the preceding clauses or a composition according to any one of the preceding clauses is administered only twice to said sow; and wherein said use does not include the administration of any other PCV3 antigen to said animal before or during the administration of said protein according to any one of the preceding clauses or a composition according to any one of the preceding clauses.

163. A protein according to any one of the preceding clauses or a composition according to any one of the preceding clauses for the use according to any one of the preceding clauses wherein said animal is a piglet; and wherein the protein according to any one of the preceding clauses or a composition according to any one of the preceding clauses is administered once to said piglet; and wherein said use does not include the administration of any other PCV3 antigen to said animal before or during the administration of said protein according to any one of the preceding clauses or a composition according to any one of the preceding clauses.

164. A protein according to any one of the preceding clauses or a composition according to any one of the preceding clauses for the use according to any one of the preceding clauses wherein said animal is a piglet; and wherein the protein according to any one of the preceding clauses or a composition according to any one of the preceding clauses is administered once to said piglet; and wherein said use does not include the administration of any other PCV3 antigen to said animal before or during the administration of said protein according to any one of the preceding clauses or a composition according to any one of the preceding clauses.

165. A protein according to any one of the preceding clauses or a composition according to any one of the preceding clauses for the use according to any one of the preceding clauses wherein said animal is a piglet; and wherein the protein according to any one of the preceding clauses or a composition according to any one of the preceding clauses is administered only once to said piglet; and wherein said use does not include the administration of any other PCV3 antigen to said animal before or during the administration of said protein according to any one of the preceding clauses or a composition according to any one of the preceding clauses.

166. A protein according to any one of the preceding clauses or a composition according to any one of the preceding clauses for the use according to any one of the preceding clauses wherein the administration to the animal in the use consists of a single, one shot administration or a single, one dose administration of said protein according to any one of the preceding clauses or a composition according to any one of the preceding clauses.

167. A protein according to any one of the preceding clauses or a composition according to any one of the preceding clauses for the use according to any one of the preceding clauses wherein the administration to the animal in the use consists of a multi-shot or multi-dose regimen of said protein according to any one of the preceding clauses or a composition according to any one of the preceding clauses.

168. A protein according to any one of the preceding clauses or a composition according to any one of the preceding clauses for the use according to any one of the preceding clauses wherein the administration to the animal in the use consists of a double shot administration; or a dual dose administration of said protein according to any one of the preceding clauses or a composition according to any one of the preceding clauses.

169. A protein according to any one of the preceding clauses or a composition according to any one of the preceding clauses for the use according to any one of the preceding clauses wherein the administration to the animal occurs within at least 1 or 2 or 3 weeks of exposure to virulent Porcine Circovirus.

170. A protein according to any one of the preceding clauses or a composition according to any one of the preceding clauses wherein the animal is a piglet not older than 15 weeks of age, or not older than 6 weeks of age, or not older than 3 weeks of age, or not older than 2 weeks of age, or not older than 1 week of age.

171. A protein according to any one of the preceding clauses or a composition according to any one of the preceding clauses for the use according to any one of the preceding clauses wherein said protein according to any one of the preceding clauses is for the use of any one of the preceding clauses.

172. A protein according to any one of the preceding clauses or a composition according to any one of the preceding clauses for the use according to any one of the preceding clauses wherein said protein according to any one of the preceding clauses is for the use of two or more uses of the preceding clauses.

173. A protein according to any one of the preceding clauses or a composition according to any one of the preceding clauses for the use according to any one of the preceding clauses wherein said composition according to any one of the preceding clauses is for the use of any one of the preceding clauses.

174. A protein according to any one of the preceding clauses or a composition according to any one of the preceding clauses for the use according to any one of the preceding clauses wherein a second antigen is administered to the animal before administration of the protein according to any one of the preceding clauses or a composition according to any one of the preceding clauses.

175. A protein according to any one of the preceding clauses or a composition according to any one of the preceding clauses for the use according to any one of the preceding clauses wherein a second antigen is administered to the animal at the same time as administration of the protein according to any one of the preceding clauses or a composition according to any one of the preceding clauses.

176. A protein according to any one of the preceding clauses or a composition according to any one of the preceding clauses for the use according to any one of the preceding clauses wherein a second antigen is administered to the animal at the same time and in the same composition as administration of the protein according to any one of the preceding clauses or a composition according to any one of the preceding clauses.

177. A protein according to any one of the preceding clauses or a composition according to any one of the preceding clauses for the use according to any one of the preceding clauses wherein a second antigen is administered to the animal at the same time and in a different composition as administration of the protein according to any one of the preceding clauses or a composition according to any one of the preceding clauses.

178. A protein according to any one of the preceding clauses or a composition according to any one of the preceding clauses for the use according to any one of the preceding clauses wherein a second antigen is administered to the animal after the administration of the protein according to any one of the preceding clauses or a composition according to any one of the preceding clauses.

179. A protein according to any one of the preceding clauses as the single PCV3 antigen for use in the vaccination of a pig to lessen the severity of clinical signs or clinical symptoms resulting from PCV3 infection in the pig, wherein the protein is in an immunogenic composition that is administered in one dose to the pig.

180. A protein according to any one of the preceding clauses as the single PCV3 antigen for use in the vaccination of a pig to lessen the severity of clinical signs or clinical symptoms resulting from PCV3 infection in the pig, wherein the protein is in an immunogenic composition that is administered in only one dose to the pig.

181. A protein according to any one of the preceding clauses as the single PCV3 antigen for use in the vaccination of a pig to lessen the severity of clinical signs or clinical symptoms resulting from PCV3 infection in the pig, wherein the protein is in an immunogenic composition that is administered in two doses to the pig.

182. A protein according to any one of the preceding clauses as the single PCV3 antigen for use in the vaccination of a pig to lessen the severity of clinical signs or clinical symptoms resulting from PCV3 infection in the pig, wherein the protein is in an immunogenic composition that is administered in only two doses to the pig.

183. A protein according to any one of the preceding clauses for use as the single PCV3 antigen for vaccination of a pig to lessen the severity of clinical signs or clinical symptoms resulting from PCV3 infection in the pig, wherein the protein is in an immunogenic composition that is administered in one dose to the pig.

184. A protein according to any one of the preceding clauses for use as the single PCV3 antigen for vaccination of a pig to lessen the severity of clinical signs or clinical symptoms resulting from PCV3 infection in the pig, wherein the protein is in an immunogenic composition that is administered in only one dose to the pig.

185. A protein according to any one of the preceding clauses for use as the single PCV3 antigen for vaccination of a pig to lessen the severity of clinical signs or clinical symptoms resulting from PCV3 infection in the pig, wherein the protein is in an immunogenic composition that is administered in two doses to the pig.

186. A protein according to any one of the preceding clauses for use as the single PCV3 antigen for vaccination of a pig to lessen the severity of clinical signs or clinical symptoms resulting from PCV3 infection in the pig, wherein the protein is in an immunogenic composition that is administered in only two doses to the pig.

187. An immunogenic composition according to any one of the preceding clauses for lessening the severity of clinical signs or clinical symptoms resulting from PCV3 infection in a pig;
wherein one dose of the immunogenic composition is administered to the pig in a vaccination method to lessen the severity of clinical signs or clinical symptoms resulting from PCV3 infection in the pig;
wherein the administration of the one dose of the immunogenic composition to the pig in the vaccination method lessens the severity of clinical signs or clinical symptoms resulting from PCV3 infection in the pig;
wherein the protein according to any one of the preceding clauses is the antigenic component in the one dose of the immunogenic composition in the vaccination method that lessens the severity of clinical signs or clinical symptoms resulting from PCV3 infection in the pig;
preferably wherein the protein is in an amount of at least 2 μg in the one dose of the immunogenic composition;
wherein the protein is the antigenic component in the vaccination method that lessens the severity of clinical signs or clinical symptoms resulting from PCV3 infection in the pig.

188. An immunogenic composition according to any one of the preceding clauses for lessening the severity of clinical signs or clinical symptoms resulting from PCV3 infection in a pig;
wherein only one dose of the immunogenic composition is administered to the pig in a vaccination method to lessen the severity of clinical signs or clinical symptoms resulting from PCV3 infection in the pig;
wherein the administration of the one dose of the immunogenic composition to the pig in the vaccination method lessens the severity of clinical signs or clinical symptoms resulting from PCV3 infection in the pig;
wherein the protein according to any one of the preceding clauses is the antigenic component in the one dose of the immunogenic composition in the vaccination method that lessens the severity of clinical signs or clinical symptoms resulting from PCV3 infection in the pig;
preferably wherein the protein is in an amount of at least 2 μg in the one dose of the immunogenic composition;
wherein the protein is the antigenic component in the vaccination method that lessens the severity of clinical signs or clinical symptoms resulting from PCV3 infection in the pig.

189. An immunogenic composition according to any one of the preceding clauses for lessening the severity of clinical signs or clinical symptoms resulting from PCV3 infection in a pig;
wherein two doses of the immunogenic composition is administered to the pig in a vaccination method to lessen the severity of clinical signs or clinical symptoms resulting from PCV3 infection in the pig;
wherein the administration of the two doses of the immunogenic composition to the pig in the vaccination method lessens the severity of clinical signs or clinical symptoms resulting from PCV3 infection in the pig;
wherein the protein according to any one of the preceding clauses is the antigenic component in the two doses of the immunogenic composition in the vaccination method that lessens the severity of clinical signs or clinical symptoms resulting from PCV3 infection in the pig;
preferably wherein the protein is in an amount of at least 2 μg in the one dose of the immunogenic composition;
wherein the protein is the antigenic component in the vaccination method that lessens the severity of clinical signs or clinical symptoms resulting from PCV3 infection in the pig.

190. An immunogenic composition according to any one of the preceding clauses for lessening the severity of clinical signs or clinical symptoms resulting from PCV3 infection in a pig;
wherein only two doses of the immunogenic composition is administered to the pig in a vaccination method to lessen the severity of clinical signs or clinical symptoms resulting from PCV3 infection in the pig;
wherein the administration of the two doses of the immunogenic composition to the pig in the vaccination method lessens the severity of clinical signs or clinical symptoms resulting from PCV3 infection in the pig;
wherein the protein according to any one of the preceding clauses is the antigenic component in the two doses of the immunogenic composition in the vaccination method that lessens the severity of clinical signs or clinical symptoms resulting from PCV3 infection in the pig;
preferably wherein the protein is in an amount of at least 2 μg in the one dose of the immunogenic composition;
wherein the protein is the antigenic component in the vaccination method that lessens the severity of clinical signs or clinical symptoms resulting from PCV3 infection in the pig.

191. The use according to any one of the preceding clauses wherein said clinical signs or symptoms are selected from the group consisting of reduction of average daily weight gain and mortality.

192. The use according to any one of the preceding clauses wherein said clinical signs or symptoms are selected from the group consisting of gross lesions, histological lesions, replication of PCV3 in a tissue, and PCV3 viremia.

193. The use according to any one of the preceding clauses wherein said clinical signs or symptoms are selected from the group consisting of development or production of a mummified, stillborn and/or weak fetus.

194. The use according to any one of the preceding clauses wherein said clinical signs or symptoms is or include expelling of a mummified, stillborn and/or weak fetus.

Clause Set E—The present invention will now be described by way of the following set of numbered clauses (Clause Set E). The disclosure in this set of clauses is equally applicable to the present invention. Likewise the disclosure in this set of clauses is equally applicable to each of the other set of clauses.

1. A porcine circovirus type 3 (PCV3) antigenic protein wherein said protein is a functional antigenic variant of PCV3 ORF2 protein.

2. A protein according to clause 1 wherein said PCV3 ORF2 protein is a protein encoded by SEQ ID No. 1.

3. A protein according to clause 1 or clause 2 wherein said protein comprises substitutions and/or extensions of PCV3 ORF2.

4. A protein according to any one of the preceding clauses wherein said functional antigenic variant is capable of a higher yield of virus-like particles (VLPs) than the protein encoded by SEQ ID No. 1.

5. A protein according to any one of the preceding clauses wherein said functional antigenic variant has one or more substitutions in the FG loop of the protein encoded by SEQ ID No. 1.

6. A protein according to any one of the preceding clauses wherein said functional antigenic variant has one or more substitutions in the FG loop of the protein encoded by SEQ ID No. 1, wherein those substitutions comprise substitutions of one or more of the S residue and/or the K residues and/or the H residue of the motif SKKKH of the FG loop of the protein encoded by SEQ ID No. 1.

7. A protein according to any one of the preceding clauses wherein said functional antigenic variant has a C terminal end that extends beyond the terminal SVL sequence of the protein encoded by SEQ ID No. 1.

8. A protein according to any one of the preceding clauses wherein said functional antigenic variant has a C terminal end that extends beyond the terminal SVL sequence of the protein encoded by SEQ ID No. 1; wherein said extension is from 1 to 30 amino acids long; and wherein said extension all of comprises part of the sequence VKININLTPPVATSRVPSRALPLRFGCGHR.

9. A protein according to any one of the preceding clauses wherein said functional antigenic variant is encodable by all or part of SEQ ID No. 1, 2, 5, 6 or 7.

10. A protein according to any one of the preceding clauses wherein said functional antigenic variant protein comprises or consists of an amino acid sequence having at least 90%, or at least 91%, or at least 92%, or at least 93%, or at least 94%, or at least 95%, or at least 96%, or at least 97%, or at least 98%, or at least 99%, or 100% sequence identity with the sequence of SEQ ID NO: 3, 4, 8, 9 or 10.

11. A nucleotide sequence encoding the protein according to any of the preceding clauses.

12. A baculovirus expression host transformed or transfected with the nucleotide sequence of clause 11.

13. A method of preparing a protein according to any one of clauses 1 to 10 comprising culturing the baculovirus expression host of claim 12 to cause expression of the protein.

14. A composition comprising a protein according to any one of clauses 1 to 10 and a carrier, diluent or excipient.

15. A composition according to clause 14 wherein the composition comprises an immunomodulatory agent.

16. A protein according to any one of clauses 1 to 10 or a composition according to any one of clauses 14 to 15 for use as a vaccine.

17. A protein according to any one of clauses 1 to 10 or a composition according to any one of clauses 14 to 15 for use in a method for eliciting an immune response or an immunological response or a protective immune or immunological response against PCV3 in swine.

18. A protein according to any one of clauses 1 to 10 or a composition according to any one of clauses 14 to 15 for use in a method of treating or preventing an infection with PCV3 in an animal; wherein said animal is swine.

19. A protein according to any one of the preceding clauses or a composition according to any one of the preceding clauses for use in immunizing an animal against PCV3; wherein said animal is swine.

20. A protein according to any one of the preceding clauses or a composition according to any one of the preceding clauses for use in a method of reducing or eliminating or abrogating PCV3 viral expression in an animal; wherein said animal is swine.

21. A protein according to any one of the preceding clauses or a composition according to any one of the preceding clauses for use in inducing the production of antibodies specific for PCV3 in an animal.

22. A protein according to any one of clauses 1 to 10 or a composition according to any one of clauses 14 to 15 for use in a method of reducing or preventing the clinical signs or clinical symptoms or disease caused by an infection with PCV3 in an animal; wherein said animal is swine.

23. The protein or composition for the use according to clause 22 wherein said clinical signs or symptoms are selected from the group consisting of reduction of average daily weight gain, mortality, gross lesions, histological lesions, replication of PCV3 in a tissue, PCV3 viremia, development or production of a mummified, stillborn and/or weak fetus, expelling of a mummified, stillborn and/or weak fetus.

24. The protein or composition for the use according to any one of clauses 16 to 23 wherein the administration to the animal in the use consists of a single, one shot administration or a single, one dose administration of said protein or said composition.

25. The protein or composition for the use according to any one of clauses 16 to 23 wherein the administration to the animal in the use consists of a double shot administration; or a dual dose administration of said protein or said composition according to any one of the preceding clauses.

26. The protein or composition for the use according to any one of clauses 16 to 25 wherein said protein or said composition is administered intramuscularly or intradermally to said animal.

27. A porcine circovirus type 3 (PCV3) antigenic protein for use as the single PCV3 antigen for use in the vaccination of a swine and/or to lessen the severity of clinical signs or clinical symptoms resulting from PCV3 infection in a swine, wherein the protein is in an immunogenic composition that is administered in only one dose to the swine; wherein said antigenic protein is PCV3 ORF2 protein or a functional antigenic variant thereof, preferably wherein said functional antigenic variant thereof is a protein according to any one of clauses 1 to 10; preferably wherein said swine is a piglet, preferably wherein said piglet is not older than 15 weeks of age.

28. A porcine circovirus type 3 (PCV3) antigenic protein for use as the single PCV3 antigen for use in the vaccination of a swine and/or to lessen the severity of clinical signs or clinical symptoms resulting from PCV3 infection in a swine, wherein the protein is in an immunogenic composition that is administered in only two doses to the swine; wherein said antigenic protein is PCV3 ORF2 protein or a functional antigenic variant thereof, preferably wherein said functional antigenic variant thereof is a protein according to any one of clauses 1 to 10; preferably wherein said swine is a sow or a pre-breeding gilt.

29. An immunogenic composition for lessening the severity of clinical signs or clinical symptoms resulting from PCV3 infection in a pig;
wherein only one dose of the immunogenic composition is administered to the pig in a vaccination method to lessen the severity of clinical signs or clinical symptoms resulting from PCV3 infection in the pig;
wherein the administration of the one dose of the immunogenic composition to the pig in the vaccination method lessens the severity of clinical signs or clinical symptoms resulting from PCV3 infection in the pig;
wherein a protein is the antigenic component in the one dose of the immunogenic composition in the vaccination method that lessens the severity of clinical signs or clinical symptoms resulting from PCV3 infection in the pig;
wherein the protein is the antigenic component in the vaccination method that lessens the severity of clinical signs or clinical symptoms resulting from PCV3 infection in the pig;
wherein said protein is a porcine circovirus type 3 (PCV3) antigenic protein;
wherein said antigenic protein is PCV3 ORF2 protein or a functional antigenic variant thereof;
preferably wherein said functional antigenic variant thereof is a protein according to any one of clauses 1 to 10.

30. An immunogenic composition for lessening the severity of clinical signs or clinical symptoms resulting from PCV3 infection in a pig;
wherein only one dose of the immunogenic composition is administered to the pig in a vaccination method to lessen the severity of clinical signs or clinical symptoms resulting from PCV3 infection in the pig;
wherein the administration of the one dose of the immunogenic composition to the pig in the vaccination method lessens the severity of clinical signs or clinical symptoms resulting from PCV3 infection in the pig;
wherein a protein is the antigenic component in the one dose of the immunogenic composition in the vaccination method that lessens the severity of clinical signs or clinical symptoms resulting from PCV3 infection in the pig;
wherein the protein is the antigenic component in the vaccination method that lessens the severity of clinical signs or clinical symptoms resulting from PCV3 infection in the pig;
wherein the pig is a piglet, preferably wherein said piglet is not older than 15 weeks of age
wherein said protein is a porcine circovirus type 3 (PCV3) antigenic protein;
wherein said antigenic protein is PCV3 ORF2 protein or a functional antigenic variant thereof;
preferably wherein said functional antigenic variant thereof is a protein according to any one of clauses 1 to 10.

31. An immunogenic composition for lessening the severity of clinical signs or clinical symptoms resulting from PCV3 infection in a pig;
wherein only two doses of the immunogenic composition are administered to the pig in a vaccination method to lessen the severity of clinical signs or clinical symptoms resulting from PCV3 infection in the pig;
wherein the administration of the two doses of the immunogenic composition to the pig in the vaccination method lessen the severity of clinical signs or clinical symptoms resulting from PCV3 infection in the pig;
wherein a protein is the antigenic component in the two doses of the immunogenic composition in the vaccination method that lessens the severity of clinical signs or clinical symptoms resulting from PCV3 infection in the pig;
wherein the protein is the antigenic component in the vaccination method that lessens the severity of clinical signs or clinical symptoms resulting from PCV3 infection in the pig;
wherein said protein is a porcine circovirus type 3 (PCV3) antigenic protein;
wherein said antigenic protein is PCV3 ORF2 protein or a functional antigenic variant thereof;
preferably wherein said functional antigenic variant thereof is a protein according to any one of clauses 1 to 10.

32. An immunogenic composition for lessening the severity of clinical signs or clinical symptoms resulting from PCV3 infection in a pig;
wherein only two doses of the immunogenic composition is administered to the pig in a vaccination method to lessen the severity of clinical signs or clinical symptoms resulting from PCV3 infection in the pig;
wherein the administration of the two doses of the immunogenic composition to the pig in the vaccination method lessen the severity of clinical signs or clinical symptoms resulting from PCV3 infection in the pig;
wherein a protein is the antigenic component in the two doses of the immunogenic composition in the vaccination method that lessens the severity of clinical signs or clinical symptoms resulting from PCV3 infection in the pig;
wherein the protein is the antigenic component in the vaccination method that lessens the severity of clinical signs or clinical symptoms resulting from PCV3 infection in the pig;
wherein the pig is a sow or a pre-breeding gilt;
wherein said protein is a porcine circovirus type 3 (PCV3) antigenic protein;
wherein said antigenic protein is PCV3 ORF2 protein or a functional antigenic variant thereof;
preferably wherein said functional antigenic variant thereof is a protein according to any one of clauses 1 to 10.

33. The immunogenic composition for the use according to any one of clauses 29-32 wherein said clinical signs or symptoms are selected from the group consisting of reduction of average daily weight gain, mortality, gross lesions, histological lesions, replication of PCV3 in a tissue, PCV3 viremia, development or production of a mummified, stillborn and/or weak fetus, expelling of a mummified, stillborn and/or weak fetus.

34. The immunogenic composition for the use according to any one of clauses 29-33 wherein said protein or said composition is administered intramuscularly or intradermally to said animal.

In a practice of any of the embodiments of the invention, the PCV3 proteins of the invention discussed throughout this disclosure, the invention comprehends nucleic acid molecules encoding the PCV3 proteins of the invention, vectors, such as baculovirus vectors (see EP 2 460 821 A2, incorporated herein by reference, along with the documents cited therein as the methods and materials therein for expressing PCV2 ORF2 capsid protein via a baculovirus expression system can be employed in the practice of the present invention to express PCV3 ORF2 capsid protein, including such a PCV3 ORF2 wild type or mutant capsid protein as herein disclosed, as well as one or more proteins of one or more porcine pathogens if desired, to include such in a composition of the invention), containing such nucleic acid molecules, and methods for producing or expressing such mutated PCV3 proteins of the invention, such as by infecting or transfecting relevant cells with the vector (e.g., if the vector be baculovirus, a relevant cell can be an insect or Sf cell or Sf+ cell; see EP 2 460 821 A2, incorporated herein by reference, along with the documents cited therein). It is advantageous to recover or isolate the protein after expression or production, e.g., separating solids and retaining liquid or supernatant that contains soluble protein (e.g., VLPs) and filtering the supernant. The supernatant containing the soluble protein (e.g., VLPs) is inactivated, advantageously with BEI, such as about 2 to 8 or about 5 mM BEI to inactivate the baculovirus. An adjuvant, advantageously about 1 mg or about 20% v/v of the Carbopol or Carbopol 971, is also added to the composition. A dosage of about 2, 4, 8 or 16 μg of the composition in a dosage of about 1 ml or about 2 ml in a single dose or a multiple dose is administered to a pig or piglet not older than 15 weeks of age, or not older than 6 weeks of age, or not older than 3 weeks of age, or not older than 2 weeks of age, or not older than 1 week of age.

The present disclosure will be further illustrated in the following Examples, which are given for illustration purposes only and are not intended to limit the disclosure in any way. Molecular cloning techniques (such as, but not limited to, construction of DNA inserts, plasmids and recombinant viral or plant vectors) were carried out using the standard molecular biology techniques described by J. Sambrook et. al. (Molecular Cloning: A Laboratory Manual, 2nd Edition, Cold Spring Harbor Laboratory, Cold Spring Harbor, New York, 1989), and in U.S. Pat. No. 8,865,183, the disclosure of which is incorporated by reference.

Although the present invention and its advantages have been described in detail, it should be understood that various changes, substitutions and alterations can be made herein without departing from the spirit and scope of the invention as defined in the appended claims.

The present invention will be further illustrated in the following Examples which are given for illustration purposes only and are not intended to limit the invention in any way.

### EXAMPLES

In the Examples presented herein, the primary data have been included in addition to the summary tables that analyse that primary data. As with any field trials, the results are not exactly the same with each animal and, in addition, there can be one or more anomalous results. However, it is to be understood that the summary tables present the analysis of the primary data. The analysis results show that the present invention is effective.

### Example 1

### Identifying and Cloning PCV3 ORF2, and Production and Purification of BaculoG/PCV3 ORF2

The PCV3 ORF2 coding sequence (SEQ ID NO:1) was cloned by PCR from a synthetic gene containing the KT869077 ORF2 sequence (see Fan et al., “Complete Genome Sequence of a Novel Porcine Circovirus Type 3 Strain, PCV3/CN/Hubei-618/2016, Isolated from China, Genome Announc 2017 Apr. 5(15) e00100-17, April 13. doi: 10.1128/genomeA.00100-17, incorporated herein reference; see also SEQ ID NO: 4; U.S. Pat. No. 10,450,351, also incorporated herein by reference) and ligated into baculovirus transfer plasmid pVL1393 (Invitrogen) utilizing 5′ BamHI and 3′ NotI restriction sites. The BamHI/NotI restriction fragment also contained a Kozak consensus sequence (GCCACC) directly between the 5′ BamHI site and the PCV3 ORF2 start codon. Recombinant baculovirus containing the PCV3 ORF2 coding sequence under the control of the polyhedron promoter was generated by co-transfection of Sf9 insect cells (Spodoptera frugiperda) with linearized baculovirus DNA and transfer plasmid pVL1393-PCV3 ORF2. The resulting recombinant baculovirus, BaculoG/PCV3 ORF2, was amplified on Sf9 insect cells and subsequently purified by limiting dilution cloning. Mention is also made as to employing the method of EP 2 460 821 A2, incorporated herein by reference, along with the documents cited therein, with the coding sequence being for a PCV3 ORF2 protein as herein disclosed (including that the foregoing methods are employed for preparing any mutant or variant or modified PCV3 ORF2 protein, especially SEQ ID NO: 3, 4, 8, 9 or 10).

The PCV3 ORF2 coding sequence (SEQ ID NO:1) was cloned by PCR from a synthetic gene containing the KT869077 ORF2 sequence and ligated into baculovirus transfer plasmid pVL1393 utilizing 5′ BamHI and 3′ NotI restriction sites. The BamHI/NotI restriction fragment also contained a Kozak consensus sequence (GCCACC) directly between the 5′ BamHI site and the PCV3 ORF2 start codon. Recombinant baculovirus containing the PCV3 ORF2 coding sequence under the control of the polyhedron promoter was generated by co-transfection of Sf9 insect cells (Spodoptera frugiperda) with linearized baculovirus DNA and transfer plasmid pVL1393-PCV3 ORF2. The resulting recombinant baculovirus, BaculoG/PCV3 ORF2, was amplified on Sf9 insect cells and subsequently purified by limiting dilution cloning.

### Examples 1A, 1B, 1C

Identifying and cloning PCV3 ORF2 and mutants or variants thereof (FG Loop mutations, FG Loop mutations and extended or added to C-terminus), production and purification of BaculoG/PCV3 ORF2 and mutants or variants thereof (FG Loop mutations, FG Loop mutations and extended or added to C-terminus), and uses thereof.

Example 1A: The nucleic acid molecule encoding the PCV3 ORF2 protein of SEQ ID NO: 4 was cloned into a vector, a baculovirus vector (see Example 1, see also EP 2 460 821 A2, incorporated herein by reference, along with the documents cited therein as the methods and materials therein for expressing PCV2 ORF2 capsid protein via a baculovirus expression system) (when desired to include such in a composition of the invention, one or more proteins of one or more porcine pathogens may be also expressed using a vector system such as a baculovirus system, or can be inactivated pathogen such as inactivated virus, e.g., PRRSV or bacterin or supernatant of bacteria culture). Cells are infected or transfected with the vector, the baculovirus vector (See Example 1, Example 2, see EP 2 460 821 A2, incorporated herein by reference, along with the documents cited therein; SF+ (Spodoptera frugiperda) cells infected or transfected at an approximate MOI of 0.076 with a recombinant baculovirus containing the coding for Porcine Circovirus 3 ORF2 gene 2 under control of the baculovirus polyhedrin promoter).

After expression or production of protein, the protein is recovered or isolated, e.g., separating solids and retaining liquid or supernatant that contains soluble protein (e.g., VLPs) and filtering the supernant. The supernatant containing the soluble protein (e.g., VLPs) is inactivated, advantageously with BEI, such as about 2 to 8 or about 5 mM BEI to inactivate the baculovirus. An adjuvant, advantageously about 1 mg or about 20% v/v of the Carbopol or Carbopol 971, is also added to make the composition. (See, e.g., Example 2, flask is incubated at 28° C.±2° C. with constant agitation at approximately 100 rpm for seven days. Cells and media are aseptically transferred to 2×1 L centrifuge bottles and cells are pelleted at 15,000×g for 20 minutes at 4° C. The resulting supernatant is 0.2 μm filtered and stored at 4° C.; inactivated Baculovirus PCV3 ORF2 Antigen, 800 mL; Carbopol 971P (0.5% stock solution) Adjuvant, 200 mL; total 1000 mL or 1 L).

A single dosage (i.e., one shot or single administration) of the composition containing either 2 μg, 4 μg, 8 μg or 16 μg of PCV3 ORF2 Antigen in a 1 ml or about 2 ml total volume is administered to groups of pigs (e.g., 6 pigs per group). A group of pigs or piglets is not older than 15 weeks of age. A group of pigs or piglets is not older than 6 weeks of age. A group of pigs or piglets is not older than 3 weeks of age. A group of pigs or piglets is not older than 2 weeks of age. A group of pigs or piglets is not older than 1 week of age. A group of pigs is sows, pre-insemination. Administration, e.g., as to timing, of single dosage is one of the below-mentioned administrations of the multiple dose regimen discussed immediately below. From the single administration, each of the groups of pigs demonstrates immunity, e.g., a protective immunity, against PCV3 and/or clinical signs or symptoms thereof, and/or reduction or lessening or prevention of PCV3 infection or incidence thereof and/or of clinical signs or symptoms thereof.

A multiple dosage regimen, i.e., two shots or two single administrations (e.g., a prime and a boost), spaced apart by at least a week of the composition containing either 2 μg, 4 μg, 8 μg or 16 μg of PCV3 ORF2 Antigen in a 1 ml or about 2 ml total volume is administered to groups of pigs (e.g., 6 pigs per group). A group of pigs or piglets is not older than 15 weeks of age (first administration at 2 or 3 weeks of age and second administration at 3 or 4 weeks of age). A group of pigs or piglets is not older than 6 weeks of age (first administration at 2 or 3 weeks of age and second administration at 3, 4 or 5 weeks of age). A group of pigs or piglets is not older than 3 weeks of age (first administration between 7 and 14 days of age, second administration between 14 and 21 days of age). A group of pigs or piglets is not older than 2 weeks of age (first administration at 1 week of age and second administration at 2 weeks of age). A group of pigs or piglets is not older than 1 week of age (administrations at days 3 or 4 and 7). A group of pigs is sows, pre-insemination (first administration between 4 and 6 weeks pre-insemination and second administration between 2 and 4 weeks pre-insemination). From the multiple administration, each of the groups of pigs demonstrates immunity, e.g., a protective immunity, against PCV3 and/or clinical signs or symptoms thereof, and/or reduction or lessening or prevention of PCV3 infection or incidence thereof and/or of clinical signs or symptoms thereof.

Example 1B: The nucleic acid molecule encoding the PCV3 ORF2 protein of SEQ ID NO: 8 (4 mutations in FG Loop; FG Loop of PCV3 ORF2 protein replaced with that of PCV2 (SKKK->QPFS) was cloned into a vector, a baculovirus vector (see Example 1, see also EP 2 460 821 A2, incorporated herein by reference, along with the documents cited therein as the methods and materials therein for expressing mutated PCV2 ORF2 capsid protein via a baculovirus expression system) (when desired to include such in a composition of the invention, one or more proteins of one or more porcine pathogens may be also expressed using a vector system such as a baculovirus system, or can be inactivated pathogen such as inactivated virus, e.g., PRRSV or bacterin or supernatant of bacteria culture). Cells are infected or transfected with the vector, the baculovirus vector (See Example 1, Example 2, see EP 2 460 821 A2, incorporated herein by reference, along with the documents cited therein; SF+ (Spodoptera frugiperda) cells infected or transfected at an approximate MOI of 0.076 with a recombinant baculovirus containing the coding for mutated Porcine Circovirus 3 ORF2 gene 2 under control of the baculovirus polyhedrin promoter).

After expression or production of mutated protein, the mutated protein is recovered or isolated, e.g., separating solids and retaining liquid or supernatant that contains soluble mutated protein (e.g., VLPs) and filtering the supernant. The supernatant containing the soluble mutated protein (e.g., VLPs) is inactivated, advantageously with BEI, such as about 2 to 8 or about 5 mM BEI to inactivate the baculovirus. An adjuvant, advantageously about 1 mg or about 20% v/v of the Carbopol or Carbopol 971, is also added to make the composition. (See, e.g., Example 2, flask is incubated at 28° C.±2° C. with constant agitation at approximately 100 rpm for seven days. Cells and media are aseptically transferred to 2×1 L centrifuge bottles and cells are pelleted at 15,000×g for 20 minutes at 4° C. The resulting supernatant is 0.2 μm filtered and stored at 4° C.; inactivated Baculovirus mutated PCV3 ORF2 Antigen, 800 mL; Carbopol 971P (0.5% stock solution) Adjuvant, 200 mL; total 1000 mL or 1 L). The amount of VLP (soluble mutated PCV3 ORF2 protein) obtained with the mutant is greater than the amount of VLP obtained from native sequence of SEQ ID NO: 4.

A single dosage (i.e., one shot or single administration) of the composition containing either 2 μg, 4 μg, 8 μg or 16 μg of mutated PCV3 ORF2 Antigen in a 1 ml or about 2 ml total volume is administered to groups of pigs (e.g., 6 pigs per group). A group of pigs or piglets is not older than 15 weeks of age. A group of pigs or piglets is not older than 6 weeks of age. A group of pigs or piglets is not older than 3 weeks of age. A group of pigs or piglets is not older than 2 weeks of age. A group of pigs or piglets is not older than 1 week of age. A group of pigs is sows, pre-insemination. Administration, e.g., as to timing, of single doseage is one of the below-mentioned administrations of the mutiple dose regimen discussed immediately below. From the single administration, each of the groups of pigs demonstrates immunity, e.g., a protective immunity, against PCV3 and/or clinical signs or symptoms thereof, and/or reduction or lessening or prevention of PCV3 infection or incidence thereof and/or of clinical signs or symptoms thereof.

A multiple dosage regimen, i.e., two shots or two single administrations (e.g., a prime and a boost), spaced apart by at least a week of the composition containing either 2 μg, 4 μg, 8 μg or 16 μg of mutated PCV3 ORF2 Antigen in a 1 ml or about 2 ml total volume is administered to groups of pigs (e.g., 6 pigs per group). A group of pigs or piglets is not older than 15 weeks of age (first administration at 2 or 3 weeks of age and second administration at 3 or 4 weeks of age). A group of pigs or piglets is not older than 6 weeks of age (first administration at 2 or 3 weeks of age and second administration at 3, 4 or 5 weeks of age). A group of pigs or piglets is not older than 3 weeks of age (first administration between 7 and 14 days of age, second administration between 14 and 21 days of age). A group of pigs or piglets is not older than 2 weeks of age (first administration at 1 week of age and second administration at 2 weeks of age). A group of pigs or piglets is not older than 1 week of age (administrations at days 3 or 4 and 7). A group of pigs is sows, pre-insemination (first administration between 4 and 6 weeks pre-insemination and second administration between 2 and 4 weeks pre-insemination). From the multiple administration, each of the groups of pigs demonstrates immunity, e.g., a protective immunity, against PCV3 and/or clinical signs or symptoms thereof, and/or reduction or lessening or prevention of PCV3 infection or incidence thereof and/or of clinical signs or symptoms thereof.

Example 1C: The nucleic acid molecules encoding (a) the mutated PCV3 ORF2 protein having 4 mutations in FG Loop; FG Loop of PCV3 ORF2 protein replaced with that of PCV2 (SKKK->QPFS) and 30 amino acid extension of C-terminus by removal of stop codon in natural PCV3 ORF2 coding sequence-term extended by removal of stop codon, i.e., after “SVL” at natural PCV3 ORF2 protein C-terminus, the addition of: VKININLTPPVATSRVPSRALPLRFGCGHR, see SEQ ID NO: 8 and 9; and (b) the mutated PCV3 ORF2 protein having 30 amino acid extension of C-terminus by removal of stop codon in natural PCV3 ORF2 coding sequence-term extended by removal of stop codon, i.e., after “SVL” at natural PCV3 ORF2 protein C-terminus, the addition of: VKININLTPPVATSRVPSRALPLRFGCGHR, see SEQ ID NO:9, each was cloned into a vector, a baculovirus vector (see Example 1, see also EP 2 460 821 A2, incorporated herein by reference, along with the documents cited therein as the methods and materials therein for expressing mutated PCV2 ORF2 capsid proteins via a baculovirus expression system) (when desired to include such in a composition of the invention, one or more proteins of one or more porcine pathogens may be also expressed using a vector system such as a baculovirus system, or can be inactivated pathogen such as inactivated virus, e.g., PRRSV or bacterin or supernatant of bacteria culture). Cells are infected or transfected with the vectors encoding (a) or (b), the baculovirus vectors encoding (a) or (b) (See Example 1, Example 2, see EP 2 460 821 A2, incorporated herein by reference, along with the documents cited therein; SF+(Spodoptera frugiperda) cells infected or transfected at an approximate MOI of 0.076 with a recombinant baculovirus containing the coding for mutated Porcine Circovirus 3 ORF2 gene 2 under control of the baculovirus polyhedrin promoter).

After expression or production of mutated proteins (a) or (b), the mutated proteins each is recovered or isolated, e.g., separating solids and retaining liquid or supernatant that contains soluble mutated protein (e.g., VLPs) and filtering the supernant. The supernatant containing the soluble mutated protein (e.g., VLPs) is inactivated, advantageously with BEI, such as about 2 to 8 or about 5 mM BEI to inactivate the baculovirus. An adjuvant, advantageously about 1 mg or about 20% v/v of the Carbopol or Carbopol 971, is also added to make the composition. (See, e.g., Example 2, flask is incubated at 28° C.±2° C. with constant agitation at approximately 100 rpm for seven days. Cells and media are aseptically transferred to 2×1 L centrifuge bottles and cells are pelleted at 15,000×g for 20 minutes at 4° C. The resulting supernatant is 0.2 μm filtered and stored at 4° C.; inactivated Baculovirus mutated PCV3 ORF2 Antigen, 800 mL; Carbopol 971P (0.5% stock solution) Adjuvant, 200 mL; total 1000 mL or 1 L). The amount of VLP (soluble mutated PCV3 ORF2 proteins) obtained with each mutant is greater than the amount of VLP obtained from native sequence of SEQ ID NO: 4. The amount of VLP (soluble mutated PCV3 ORF2 protein) obtained with the mutant having both the FG Loop mutation and the extension (mutant (b) can be greater than the amount of VLP obtained from the FG Loop mutant or variant alone or the extension alone.

A single dosage (i.e., one shot or single administration) of the composition containing either 2 μg, 4 μg, 8 μg or 16 μg of either mutated PCV3 ORF2 Antigen (a) or (b) in a 1 ml or about 2 ml total volume is administered to groups of pigs (e.g., 6 pigs per group). A group of pigs or piglets is not older than 15 weeks of age. A group of pigs or piglets is not older than 6 weeks of age. A group of pigs or piglets is not older than 3 weeks of age. A group of pigs or piglets is not older than 2 weeks of age. A group of pigs or piglets is not older than 1 week of age. A group of pigs is sows, pre-insemination. Administration, e.g., as to timing, of single dosage is one of the below-mentioned administrations of the multiple dose regimen discussed immediately below. From the single administration of each of (a) or (b) in the dosages, each of the groups of pigs demonstrates immunity, e.g., a protective immunity, against PCV3 and/or clinical signs or symptoms thereof, and/or reduction or lessening or prevention of PCV3 infection or incidence thereof and/or of clinical signs or symptoms thereof.

A multiple dosage regimen, i.e., two shots or two single administrations (e.g., a prime and a boost; or same mutant, i.e., prime and boost are either with both (a) or both (b), and prime and boost are in same dosage amount), spaced apart by at least a week of the composition containing either 2 μg, 4 μg, 8 μg or 16 μg of mutated PCV3 ORF2 Antigen (a) or (b) in a 1 ml or about 2 ml total volume is administered to groups of pigs (e.g., 6 pigs per group). A group of pigs or piglets is not older than 15 weeks of age (first administration at 2 or 3 weeks of age and second administration at 3 or 4 weeks of age). A group of pigs or piglets is not older than 6 weeks of age (first administration at 2 or 3 weeks of age and second administration at 3, 4 or 5 weeks of age). A group of pigs or piglets is not older than 3 weeks of age (first administration between 7 and 14 days of age, second administration between 14 and 21 days of age). A group of pigs or piglets is not older than 2 weeks of age (first administration at 1 week of age and second administration at 2 weeks of age). A group of pigs or piglets is not older than 1 week of age (administrations at days 3 or 4 and 7). A group of pigs is sows, pre-insemination (first administration between 4 and 6 weeks pre-insemination and second administration between 2 and 4 weeks pre-insemination). From the multiple administration or either (a) or (b), each of the groups of pigs demonstrates immunity, e.g., a protective immunity, against PCV3 and/or clinical signs or symptoms thereof, and/or reduction or lessening or prevention of PCV3 infection or incidence thereof and/or of clinical signs or symptoms thereof.

### Example 2

### Production of BaculoG/PCV3 ORF2 Antigen for the Study

A 1 L lot of antigen was produced in a 3L spinner flask by infecting SF+ (Spodoptera frugiperda) cells at an approximate MOI of 0.076 with a recombinant baculovirus containing the Porcine Circovirus 3 ORF2 gene 2 under control of the baculovirus polyhedrin promoter (BaculoG/PCV3 ORF2 Clone 4B4-2E12 Pre-MSV p8). The flask was incubated at 28° C.±2° C. with constant agitation at approximately 100 rpm for seven days. Cells and media were aseptically transferred to 2×1 L centrifuge bottles and cells were pelleted at 15,000×g for 20 minutes at 4° C. The resulting supernatant was 0.2 μm filtered and stored at 4° C.

TABLE 1
Formulation of PCV3 ORF2 inactivated baculovirus vaccine

| Component | Purpose | Volume |
|---|---|---|
| Inactivated Baculovirus PCV3 ORF2 | Antigen | 800 mL |
| Carbopol 971P (0.5% stock solution) | Adjuvant | 200 mL |

### Example 3

### Efficacy Evaluation of Prototype Vaccines for Porcine Circovirus Type 3 (PCV3) in Caesarian-Derived Colostrum-Deprived Pigs

The objectives of the Example are to: evaluate the efficacy of prototype PCV3 vaccines in caesarian-derived colostrum-deprived (CDCD) pigs, develop a challenge model for PCV3 in CDCD pigs including defining primary and secondary outcome variables, confirm infectivity of infectious molecular clones.

This study was designed to evaluate the use of whole virus and PCR positive tissue homogenate (both provided by Iowa State University Veterinary Diagnostic Laboratory (ISU VDL)) as potential challenge materials for future studies. In addition, the rescue of a PCV3 infectious clone in pigs would provide an additional option for future challenge model studies and was therefore incorporated into the study design. As prototype vaccines were available, they were included to provide a stronger evaluation of the challenge model.

TABLE 2
Study design

| | | | Vaccination | Challenge | Necropsy | Necropsy |
|---|---|---|---|---|---|---|
| | | | (D0; at 3 | (D21; at 6 | (D49; at 10 | (D63; at 12 |
| [symbol] | N | Room* | weeks of age) | weeks of age) | weeks of age) | weeks of age) |
| [symbol] | 8 | A | BaculoG/PCV3- | Whole virus + | All | Not |
| | | | ISA | KLH | remaining | applicable |
| [symbol] | 8 | | BaculoG/PCV3- | | animals | |
| | | | Carbopol | | euthanized; | |
| [symbol] | 8 | | Placebo | | tissue | |
| | | | | | collection | |
| [symbol] | 12 | B | BaculoG/PCV3- | PCR + tissue | 8 animals | 4 animals |
| | | | Carbopol | homogenate + | euthanized; | euthanized; |
| [symbol] | 12 | | Placebo | KLH | tissue | tissue |
| | | | | | collection | collection |
| [symbol] | 6 | C | None | Placebo | 3 animals | 3 animals |
| | | | | challenge | euthanized; | euthanized; |
| | | | | controls | tissue | tissue |
| | | | | (whole virus | collection | collection |
| | | | | media) | | |

| | | | Challenge | |
|---|---|---|---|---|
| [symbol] | N | Room | (at D14; at 5 wks) | Necropsy |
| [symbol] | 2 | D | Infectious clone-BIAH re-circularized genome | All animals |
| [symbol] | 2 | | Infectious clone-ISU re-circularized genome | euthanized at |
| [symbol] | 2 | | Infectious clone-ISU dimerized genome in plasmid | D28 or D42 |

[symbol]

indicates data missing or illegible when filed

A total of 54 pigs were used. The animals were randomized into five treatment groups (n=8-12/group) and one strict control group (n=6). Animals were housed in three rooms. At 7 days of age, pigs were vaccinated with PCV2. On D0, at three weeks of age, pigs were vaccinated with either a vectored construct expressing PCV3 ORF2 adjuvanted with ISA 207VG, a vectored construct expressing PCV3 ORF2 adjuvanted with carbopol, or a placebo (matched control for vectored construct). Pigs were moved at approximately five weeks of age. On D21, at six weeks of age, pigs were challenged with either whole virus or tissue homogenate. An immunostimulant (IFA/KLH) was administered in addition to the challenge material. As used herein, the role of the immune stimulant was not of an adjuvant, but as a challenge enhancer. Rectal temperatures, body weight, serum, whole blood, nasal swabs, and fecal swabs were collected periodically throughout the study. Samples were tested jointly. Animals were euthanized at either D49 or D63 as described in Table 2. Multiple fresh and fixed tissues were collected and evaluated.

For the investigation with infectious clone constructs, a total of 6 pigs were used. The animals were randomized into three groups (n=2/group) and housed in a single room. At D14 when animals were approximately 5 weeks of age, they were inoculated with one of three infectious clone constructs. Inoculation was done intrahepatically (ultrasound-guided). In addition, animals in Group 9 were inoculated intramuscularly. Rectal temperatures, body weight, serum, nasal, and fecal samples were collected periodically throughout the study. Samples were tested by qPCR to determine whether clones were able to replicate. Animals were euthanized on D49. Multiple fresh and fixed tissues were collected only from animals that were viremic and were transferred for evaluation.

A schedule of events for the study is shown in Table 3.

TABLE 3

| Study Day | Study Event |
|---|---|
| D-22 | Collection of cord blood |
| D-14 | Vaccination of animals for PCV2 at 7 days |
| | of age |
| D0 | Vaccination of animals in groups 1-6 (3 |
| | weeks of age) |
| | Blood collection (Note: no fecal swabs, |
| | nasal swabs, temperatures or weight data |
| | collected) |
| D12 | Transport of animals |
| D14 | Challenge of animals in groups 7-9 (5 |
| | weeks of age) |
| D19 | Administration KLH/ICFA to animals in |
| | groups 1-6 |
| D21 | Challenge of animals in groups 1-5 (6 |
| | weeks of age) |
| D23 | Administration KLH/ICFA to animals in |
| | groups 1-6 |
| D49 | Necropsy selected animals in groups 4-6; |
| | all animals in group 1-3, 7-9 |
| D63 | Necropsy of remaining animals in groups |
| | 4-6 |
| D21 through D12 | General health observations on all animals |
| D13-D63 | Clinical observations on all available |
| | animals |
| D13, 15, 16, 19, 21, 23, 28 | Rectal temperature from groups 7-9 |
| | Blood collection, fecal swabs, nasal swabs |
| | in animal from groups 7-9 |
| D13, 21, 22, 23, 26, 28, 35, | Rectal temperature from groups 1-5 |
| 42, 49 | Blood collection, fecal swabs, nasal swabs |
| | in animals from groups 1-5 |
| D13, 15, 16, 19, 21, 22, 23, | Rectal temperature from group 6 |
| 26, 28, 35, 42, 49 | Blood collection, fecal swabs, nasal swabs |
| | in animals from group 6 |
| D13, 21, 28, 35, 42, 49 | Body weights (all available animals) |

An experimental vaccine (BaculoG/PCV ORF2) was compared with a placebo-matched control. Treatments are outline in Table 4.

TABLE 4

| Group | Treatment |
|---|---|
| 1 | BaculoG/PVC3 ORF2, P9; live, adjuvanted with 50% ISA |
| | 207VG; L#3624-171 |
| 2 & 4 | BaculoG/PVC3 ORF2, P9; live, adjuvanted with 20% carbopol; |
| | L#3624-172A |
| 3 & 5 | BaculoG/no insert control; P4; live, adjuvanted with 20% |
| | carbopol; L#3624-172B |
| 6 | No treatment |
| 7 | Infectious clone-BIAH re-circularized genome; Lot#3718-050 |
| 8 | Infectious clone-ISU dimerized genome in plasmid |
| 9 | Infectious clone-ISU rescued virus |

The vaccines were administered on D0 intramuscularly into the right side of the neck (2 mL), midway between the base of the ear and point of the shoulder, using appropriately-sized sterile needles and syringes. Commercial PCV2 vaccine (Circoflex, serial #3091134A) was administered to all animals per manufacturer's instructions.

Whole virus challenge: Challenge material was stored at −70° C.±10° C. until use. Immediately prior to challenge, material was thawed at 37° C. and used undiluted. Dosage was 2 mL total (1 mL IN/1 mL IM). On D21, each pig received 1 mL of viral harvest intranasally and 1 mL intramuscularly. Administration of challenge material intramuscularly was done by injecting the viral harvest into the left side of the neck, midway between the base of the ear and point of the shoulder, using appropriately-sized sterile needles and syringes. Administration of the challenge material intranasally was done by attaching a nasal tip atomizer to a 5cc luer lock syringe. Duration of challenge was 28 days. Routine culture of the material was done on blood agar plates at 37° C. anaerobically and aerobically for 48 hrs. No growth was observed and the test was considered satisfactory. The material was tested by PCR for the presence of mycoplasma; no contamination was identified. The PCV3 qPCR result was: 6.6 log 10 genomic copies/mL (Cq=23.58). Deep sequencing was completed on the samples (MiSeq_127) using both DNA and RNA processing. Sequencing did not result in recovery of PCV3.

Challenge by PCV3 PCR positive tissue homogenate. Challenge material was stored at −70° C.±10° C. until use. Immediately prior to challenge, material was thawed at 37° C. and used undiluted. Dosage was 2 mL total (1 mL IN/1 mL IM). On D21, each pig received 1 mL of viral harvest intranasally and 1 mL intramuscularly. Administration of challenge material intramuscularly was done by injecting the viral harvest into the left side of the neck, midway between the base of the ear and point of the shoulder, using appropriately-sized sterile needles and syringes. Administration of the challenge material intranasally was done by attaching a nasal tip atomizer to a 5cc luer lock syringe. Duration of challenge was 28 days. Routine culture of the material was done on blood agar plates at 37° C. anaerobically and aerobically for 48 hrs. No growth was observed and the test was considered satisfactory. The material was tested by PCR for the presence of mycoplasma; no contamination was identified. The PCV3 qPCR result was: 9.1 log 10 genomic copies/mL (Cq=14.82). Deep sequencing was completed on the samples (MiSeq_127) using both DNA and RNA processing. Sequencing resulted in recovery of the full PCV3 genome (99% nt to PCV3 GB MG564174.1).

Table 5 describes the immunostimulant given to the animals.

TABLE 5

| | |
|---|---|
| Generic Name: | Keyhole limpet hemocyanin emulsified in incomplete |
| | Freund's adjuvant (KLH/ICFA) |
| Formulations | BIVI-R&D formulated KLH/ICFA to contain the |
| (per dose): | equivalent of 1 mg KLH/1 mL adjuvanted with 1 mL |
| | of ICFA. |
| Manufacturer: | BI AH USA-Ames, IA |
| Lot Number: | 3519-049 |
| Expiration Date: | N/A |
| Storage: | Stored at 2-8° C. prior to use. |
| Presentation: | 52 mL in 60 mL plastic bottle |
| Testing: | KLG/ICFA was tested for sterility |
| Applied Dose: | 2.0 mL in the right ham muscle on D19 and 2.0 mL |
| | in the left ham muscle on D23. Treatments were |
| | administered by a Dose Administrator, a person not |
| | responsible for collecting data for this study. |
| | KLH administration was documented on the Product |
| | Dosing Record. |

On D14, pigs in Groups 7 and 8 were infected via ultrasound guided injection into the liver only—lymph nodes were not inoculated. For challenge, 1 mL of material was drawn up into a tuberculin syringe and attached to a sterile 22 g×1.5 inch needle. The needle was directed into three different areas within the liver. Approximately 300 μl was administered into each location. Pigs in Group 9 were administered inoculum as described above. In addition, they were intramuscularly injected with a total of 3 mL of material; 1.5 mL of material in the musculature of the right neck and 1.5 mL of material into the musculature of the left neck. Following challenge, pigs were administered 0.5 mL of Baytril into the musculature of the right neck. Group 7 (pigs 1 and 2) were administered material with a re-circularized genome. Group 8 (pigs 3 and 4) were administered a dimerized plasmid. Group 9 (pigs 5 and 6) were administered a transfection cell culture harvest. Table 6 shows the inclusion/exclusion criteria used in the study.

TABLE 6

| Specifications | Requirements |
|---|---|
| Species & Breed: | Porcine, CDCD |
| Age: | Pigs were 21 days of age at D0 |
| Weight Range: | No specified weight range was required |
| Source & Ownership: | Source: Struve Labs International; 1603 |
| | Enterprise St., Manning Iowa 51455 |
| | Ownership: Boehringer Ingelheim Animal |
| | Health USA, Inc. |
| Number: | 60 |
| Identification: | Ear tag (uniquely numbered) |
| Physiological status: | All pigs were vaccinated for PCV2 prior to |
| | shipment to AMVC. All piglets were |
| | healthy at the time of vaccination as |
| | determined by observation by the Study |
| | Investigator. |
| Serological status: | Not specified. |
| Additional inclusion | Serum samples collected on D0 and D13 |
| requirements: | were tested for the presence of PCV3 and |
| | PCV2 DNA by qPCR. No PCV3 or PCV2 |
| | DNA was detected at either time-point |
| Exclusion: | A total of 60 animals were transferred and |
| | there were no mortalities following |
| | transfer. All animals were included in the |
| | study. |
| Post-inclusion | No animals were removed following |
| removal: | inclusion into the study. |

The pig was the experimental unit. The randomization of pigs to pen and treatment was conducted by a statistician or designee. Prior to the start of the study, the available pigs, litter information, and housing facility set-up were used to assign treatments randomly within litter. A total of four litters ranging from 12 to 14 pigs were included for Groups 1-6. A total of two litters with three pigs were included for Groups 7-9. Personnel involved with collecting data or performing laboratory assays were blinded to the allocation of pigs to groups throughout the study. Treatments were administered by an individual not involved with data collection. The use of animals in this study was approved. Adequate floor and feeder space was provided in accordance with acceptable animal husbandry practices. Pigs were observed daily to ensure access to an adequate supply of feed and water and to determine the animals' general health. The animals were under veterinary supervision upon arrival at the facility until the end of the study. No treatments were administered to animals throughout the duration of the study. Throughout the study pigs were feed the following medicated feeds: UltraCare 100 Medicated (Lot #7November03); UltraCare 240 Medicated (Lot #8June25); UltraCare 500 Medicated (Lot #8August30); or Lean Metrics CEPS Medicated (Lot #08November14). Animals were disposed of via rendering following the conclusion of the study with the exception of animal #13 which was incinerated on D46.

All pigs were observed daily for general health from D1 through D12. No abnormalities were noted. Beginning on D13 and continuing through the end of the study, all pigs were observed daily for the presence of clinical signs as described in Table 7.

TABLE 7

| Score | Respiratory Signs | Neurological Signs | Body Condition | Diarrhea |
|---|---|---|---|---|
| 0 | Normal | Normal | Normal | Normal |
| 1 | Mild = mild | Depressed = depressed | Mild = depressed | Mild = slightly |
| | increase in | to lethargic, requires | appetite but still | loose stool |
| | respiratory rate | physical stimulation | eating, slightly thin | observed from pig |
| | | to provoke locomotion | compared to pen | |
| | | | mates | |
| 2 | Moderate = notable | Ataxic = unable | Moderate = not | Moderate = runny, |
| | increase in | to coordinate muscle | eating, ribs and | loose stool |
| | respiratory rate | activity, spastic | backbone obviously | observed; obvious |
| | | movements | pronounced | staining of the |
| | | involving head, | | perianal region |
| | | limbs, and/or trunk | | |
| 3 | Severe = thumping | Tremors = involuntary | Severe = emaciated | Severe = very |
| | | repetitive muscle | | watery stool |
| | | movements | | observed |
| 4 | | Recumbent = laying | | |
| | | down, unable to | | |
| | | raise when provoked | | |
| | | with physical stimulus | | |
| 5 | | Seizures = bilateral | | |
| | | tonic or clonic | | |
| | | contraction of | | |
| | | muscles resulting in | | |
| | | partial or complete | | |
| | | unconsciousness | | |

On the days of temperature collection, the body temperature of each animal was collected using a microchip (Destron Fearing LifeChip® with bio-Thermo Technology) and an Allflex thermometer (Model number RS420-45, serial no. C088 26001). Data was recorded in ° F. For statistical analysis, data was baseline corrected. Pyrexia was defined as a temperature greater than 104° F. On the days of body weight collection, weights were recorded in kilograms using a calibrated scale.

On blood collection dates, venous whole blood was collected via the anterior vena cava from each pig using an appropriately sized sterile Vaccutainer® needle, a Vaccutainer® needle holder, and serum separator tubes (SST). The blood was hand delivered and serum was decanted into two screw-cap cryogenic vials and one 5 mL Falcon tube labeled with at least study number, day of study, and animal ID. Serum samples in cryogenic vials were stored at −70° C.±10° C. and tracked via Freezerworks electronic management system. Serum was tested by qPCR for the presence of PCV3. The 5 mL Falcon tubes were transferred for ELISA testing.

Swab samples were collected from pigs. A separate, sterile, swab (Fisher catalog no. 23-400-111 or similar) was used to obtain a fecal sample from the rectum of the animal or a nasal sample from one nostril. Upon sampling, each swab was placed in a tube containing 1.0 mL of minimal essential media (SAFC cat #62892-1000M3056). Tubes of media were prepared and were stored at 4° C. prior to use. Following use, tubes were labeled with a minimum of animal id, study number and date. Tubes were stored at −70° C.±10° C. and delivered on the day of collection and were processed using routine methods. Processed materials were stored in vials labeled with at least study number, day of study, and animal ID. Samples were stored at −70° C.±10° C. and tracked via Freezerworks electronic management system. Samples were tested by qPCR.

Animals in Groups 7-9 were necropsied on D49. Animals in Groups 1-6 were euthanized at either D49 or D63. At the time of necropsy, macroscopic lesions were recorded on the Necropsy Report Record. The study investigator or designee collected formalin-fixed tissue samples of cerebrum (½ of the organ), cerebellum (½ of organ), brainstem (½ of organ), lung (1 section of accessory lobe or area with lesion), heart (2 sections), kidney (1 section), liver (1 section), spleen (1 section), tonsil (½ organ), small intestine (3 sections), colon (2 sections), and lymph nodes (superficial inguinal, tracheobronchial, iliac, mesenteric, gastrohepatic, and iliocecal). All fixed tissues were placed into one container containing 10% buffered formalin solution such that there was a 1:10 ratio of fixed tissue to formalin. For each pig, a replicate sample of sections listed above was collected into the following whirl pack bags; 1—cerebrum, cerebellum, brainstem; 2—lung, heart, kidney, liver, spleen, 3—lymph nodes and tonsil, 4—small intestine and colon. Bags containing fresh tissues and the jar of fixed tissues were labeled with at least study number, day of study, and animal ID. All fresh tissues were transferred on either D49 or D63. Note, no tissues were collected from animals 1 and 2 (Group 7); 4 (Group 8); or 5 and 6 (Group 9) as viremia was not detected by qPCR.

Terminal blood was collected from the following animals at D63: 57 and 55 (Group 6); 53, 50, 46, and 44 (Group 5); 41, 37, 35, and 33 (Group 4). The pigs were deeply anesthetized prior to blood collection. Blood was collected into SST tubes and delivered on the day of collection. The serum was separated from the clot by centrifugation and decanted into 50 mL centrifuge tubes labeled with at least study number, day of study, and animal ID. Serum samples were tracked via FreezerWorks electronic management system. One half of the serum collected from each animal was transferred.

Statistical analysis of data was conducted using SAS version 9.2 or higher (SAS, Cary, North Carolina/USA, SAS Institute, Inc.). Data listings and summary statistics by treatment group were generated for all variables, as appropriate. Viremia data from Groups 1-5 was dichotomized to a binary outcome (present/absent) for each animal and median PCR values by group and day were plotted. The proportion of affected animals was analyzed with a Fisher's Exact comparison between treatment groups; p-values less than 0.01 were considered significant. Fecal and nasal shedding data from Groups 1-5 was dichotomized to a binary outcome (present/absent) for each animal and median PCR values by group and day were plotted. The proportion of affected animals was analyzed with a Fisher's Exact comparison between treatment groups; p-values less than 0.01 were considered significant. The proportion of affected animals for Groups 4 and 5 by day was analyzed with a Wilcoxon test. Rectal temperatures and body weights were analyzed using a mixed model with baseline adjustment. Least-square means by group and day are reported. Group comparisons by day were analyzed; p-values less than 0.01 were considered significant.

There were three amendments to the protocol. First, due to the small size of the pigs, the protocol for inoculation of the infectious clone material was modified. Second, additional bleed dates were added for pigs in Groups 7, 8, 9 based on PCR results. Dates added included: D36, D42, and D49. In addition, the necropsy date was performed on D49 instead of on D42. Third, it was recommended by the Study Investigator that weight and temperature should not be collected on D0 and blood not be collected on D7 due to the additional stress it would place on the animal.

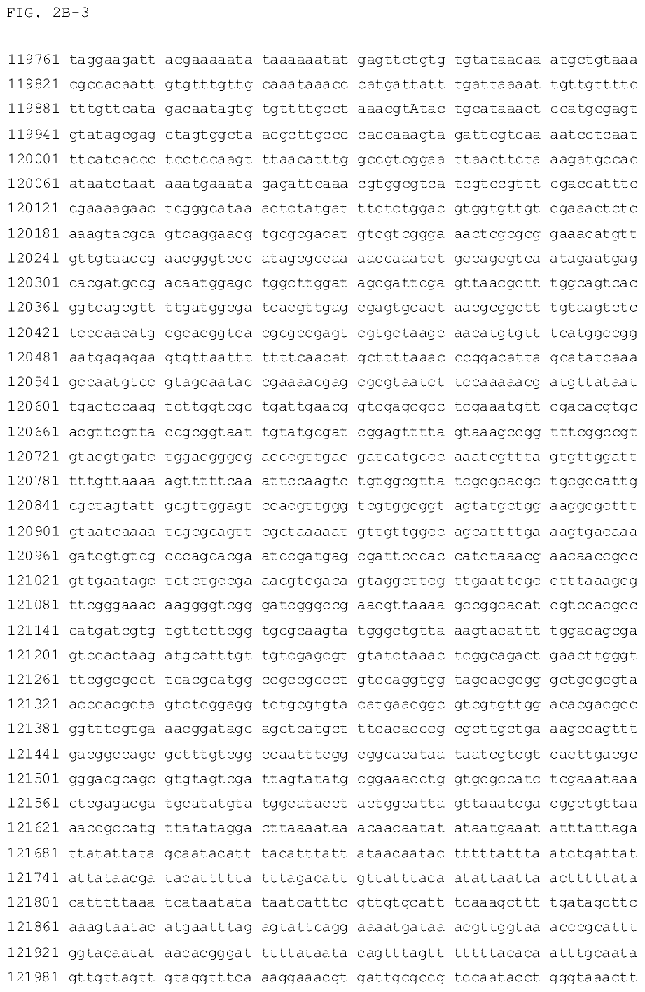

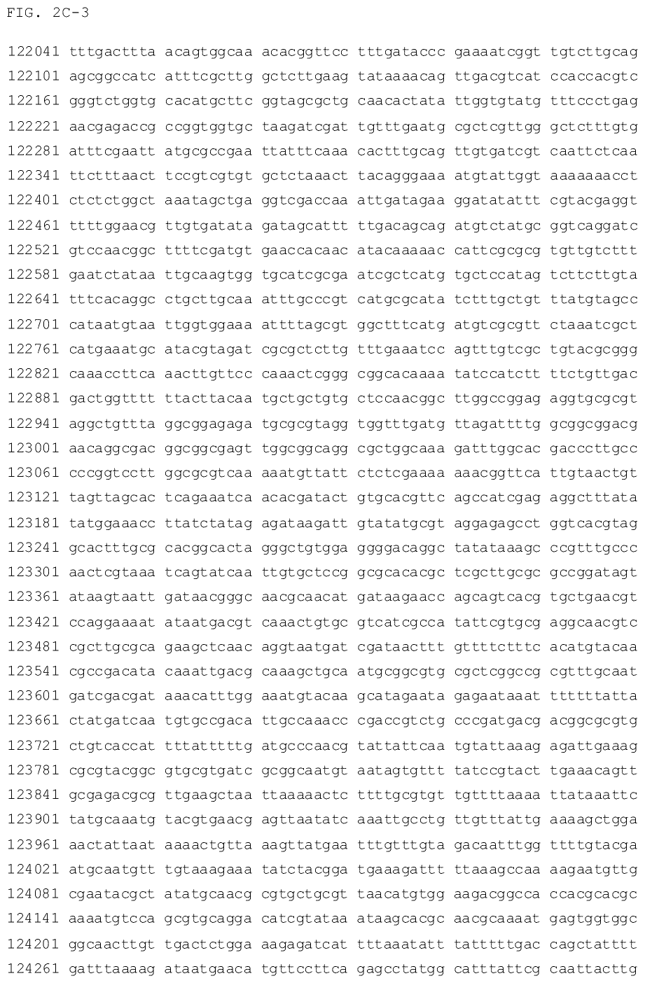

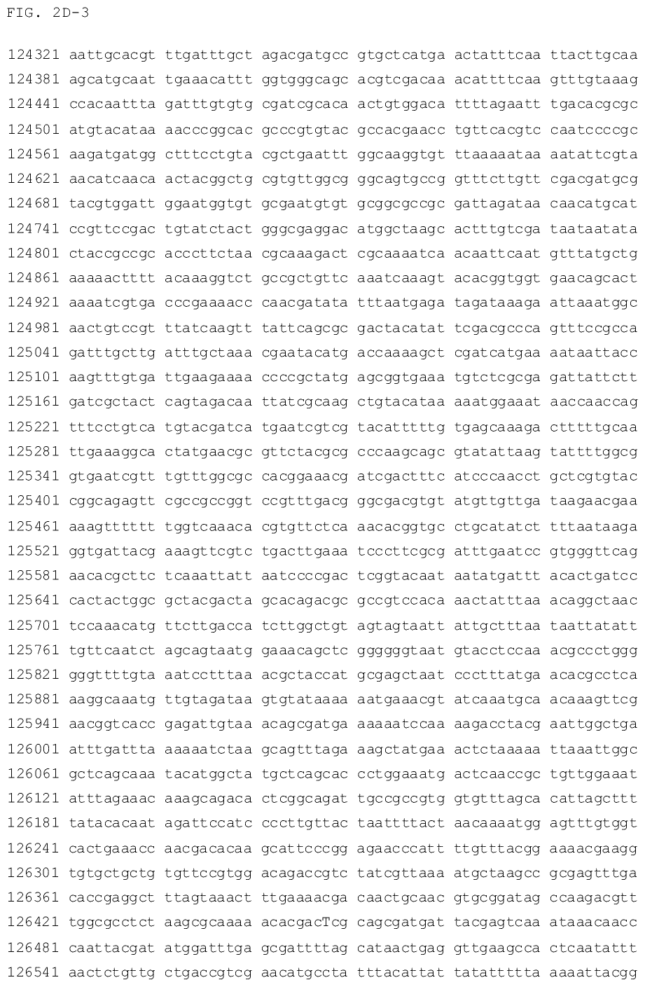

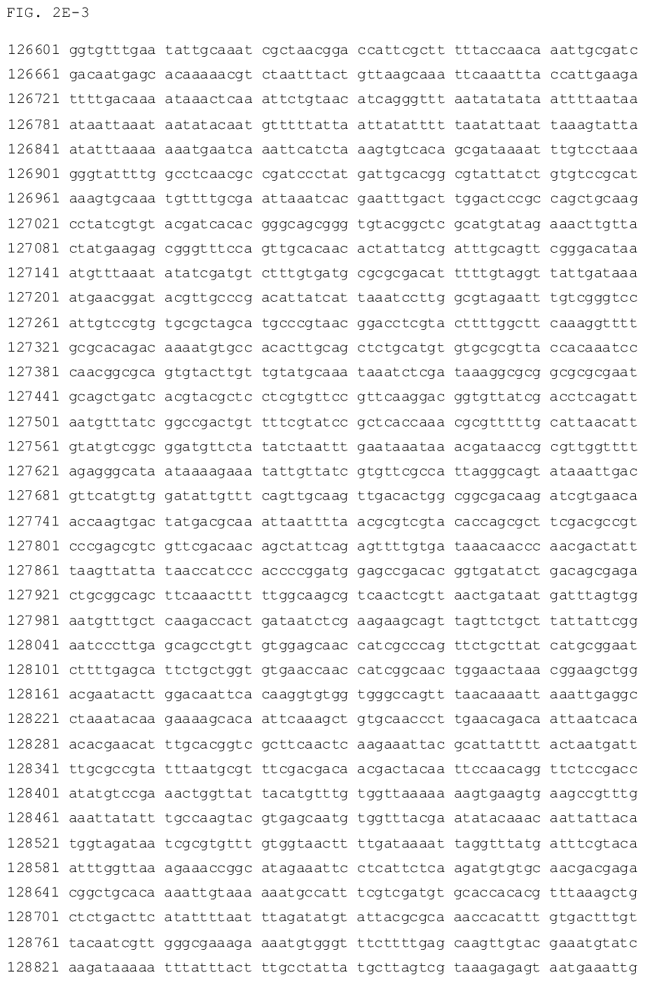

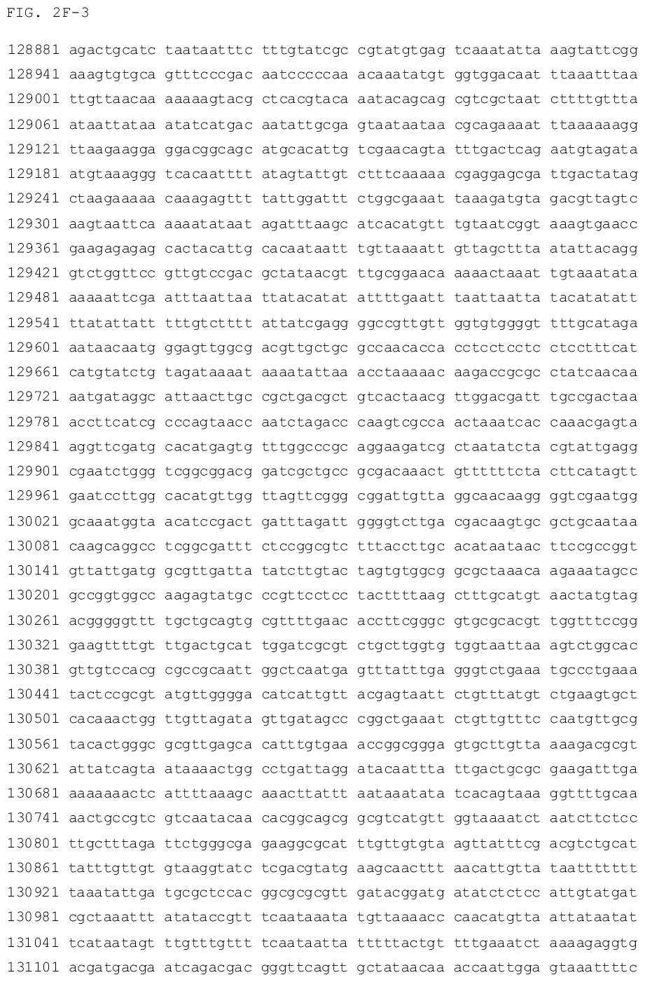

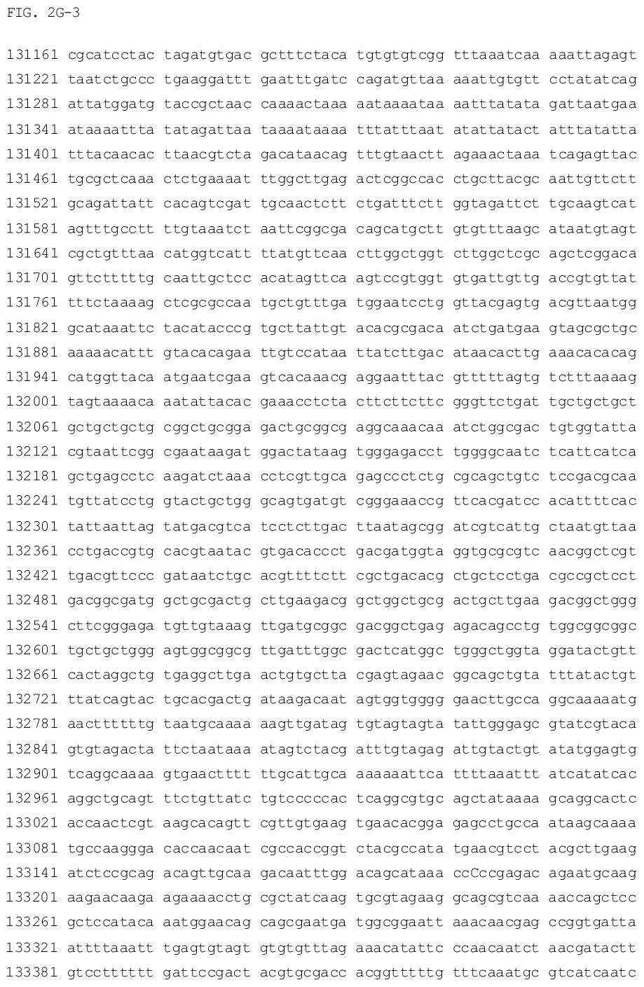

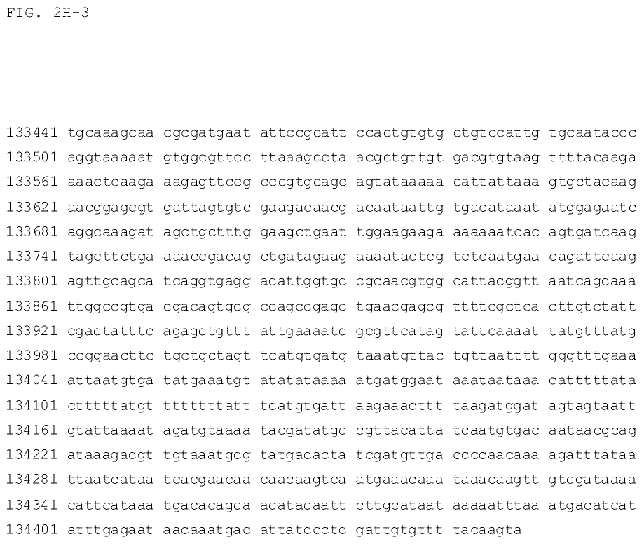

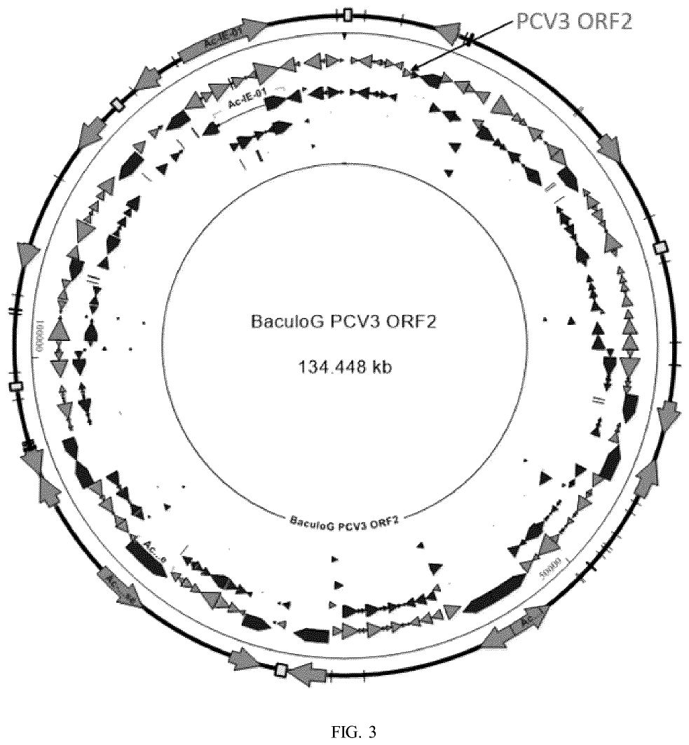

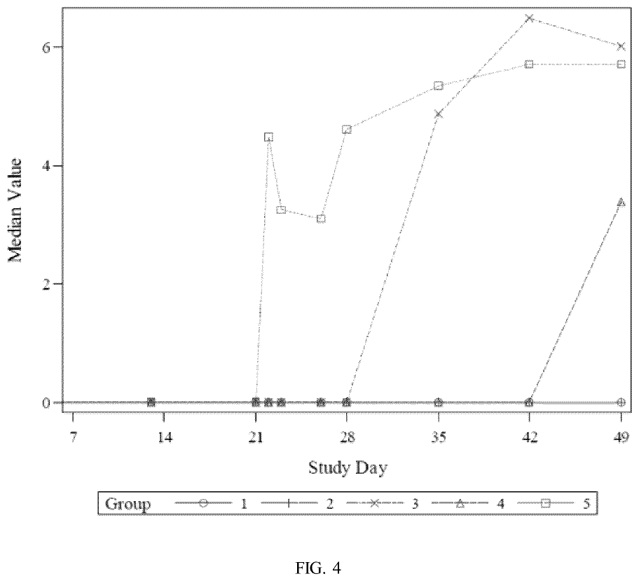

Viremia was not detected in any of the six strict control animals throughout the study (Group 6). Frequency distributions of viremia by group are presented in Table 8 below. Group median log 10 PCV3 DNA genomic copies/mL by day for Groups 1-5 are presented in FIG. 4.

In non-vaccinated pigs, exposure to the whole virus (WV) challenge material resulted in viremia in 100% of animals (Groups 3). Viremia in these animals was first observed between D28 and D42 and was present in all animals at the time of off-test (D49). In contrast, viremia was prevented in 94% (15/16) of vaccinated animals exposed to the whole virus challenge (p<0.001). The one vaccinated animal observed with viremia (#14) was in Group 1 and had detectable viremia at D49 only.

In non-vaccinated pigs, exposure to the tissue homogenate (TH) challenge material resulted in viremia in 100% of animals (Group 5). Viremia in these animals was first observed on D22 (in all animals) and was present in all animals at the time of off-test (D49). The four animals (#53, 50, 46, and 44) which were held for an additional two weeks had detectable levels of viremia at the time of necropsy on D63. In contrast, viremia was prevented in 42% (5/12) of vaccinated animals exposed to the tissue homogenate challenge (p=0.0373). Of the seven vaccinated animals that became viremic, only one animal (#40) had viremia from D22 through D49. Viremia occurred between D35 and D49 in the remaining six vaccinated animals. Table 8 shows the frequency of PCV3 DNA detection in serum by treatment group.

TABLE 8

| | | Viremia detected (ever) | | | |
|---|---|---|---|---|---|
| Group | Treatment | No | Yes | Total | % positive |
| 1 | BaculoPCV3/ISA-WV | 7 | 1 | 8 | 12.5% |
| 2 | BaculoPCV3/Carb-WV | 8 | 0 | 8 | 0.0% |
| 3 | Placebo/Carb-WV | 0 | 8 | 8 | 100.0% |
| 4 | BaculoPCV3/Carb-TH | 5 | 7 | 12 | 58.3% |
| 5 | Placebo/Carb-TH | 0 | 12 | 12 | 100.0% |
| 6 | Strict control | 6 | 0 | 6 | 0.0% |

As only two animals per group were included in the infectious clone portion of the study, raw data by animal and day is presented Table 9. PCV3 DNA was detected in both animals in Groups 7 and 8, but in only one animal from Group 9. Only one animal (#3; Group 8) developed viremia for consecutive weeks. Interestingly, viremia did not begin until D28.

TABLE 9
Log10 PCV3 DNA genomic copies/ML by animal and day

| Group | Treatment | Animal | D13 | D15 | D16 | D19 | D21 | D23 | D28 | D35 | D42 | D49 |
|---|---|---|---|---|---|---|---|---|---|---|---|---|
| 7 | IC-BI AH | 1 | 0.00 | 3.37 | 0.00 | 0.00 | 0.00 | 0.00 | 0.00 | 0.00 | 0.00 | 0.00 |
| | USA re- | 2 | 0.00 | 3.84 | 2.58 | 0.00 | 0.00 | 0.00 | 0.00 | 0.00 | 0.00 | 0.00 |
| | circularized | | | | | | | | | | | |
| | genome | | | | | | | | | | | |
| 8 | IC-ISU | 3 | 0.00 | 2.87 | 0.00 | 0.00 | 0.00 | 0.00 | 3.88 | 6.03 | 6.51 | 5.93 |
| | VDL | 4 | 0.00 | 3.41 | 0.00 | 0.00 | 0.00 | 0.00 | 0.00 | 0.00 | 0.00 | 0.00 |
| | dimerized | | | | | | | | | | | |
| | genome | | | | | | | | | | | |
| 9 | IC-ISU | 5 | 0.00 | 2.81 | 0.00 | 0.00 | 0.00 | 0.00 | 0.00 | 0.00 | 0.00 | 0.00 |
| | VDL | 6 | 0.00 | 0.00 | 0.00 | 0.00 | 0.00 | 0.00 | 0.00 | 0.00 | 0.00 | 0.00 |
| | transfection | | | | | | | | | | | |
| | harvest | | | | | | | | | | | |

No clinical signs were observed in any animal following vaccination through D12 (day of transport). Throughout the study, only two animals (#13, Group 1; #59, Group 6) had ongoing abnormalities. Three additional animals were observed to have sporadic abnormalities.

Animal #13 (Group 1) was observed to have pronounced ribs and backbone and was not eating (body condition score of 2) shortly after arrival on D13 and 14. On D23, the animal was uncoordinated following bleeding. On D28, the animal was noted to have a lame left rear leg. The animal was found dead on D46. Macroscopic examination at the time of death revealed fibrinous pleuritis with multifocal areas of atelectasis in the cranial ventral lung lobes and fibrinous pericarditis. Based on the gross lesions, death was secondary to a systemic bacterial infection. The death was likely unrelated to vaccination or challenge as PCV3 DNA was not detected in serum from this animal at any point during the study.

Animal #59 (Group 6) was observed to be lame on the right rear leg from D32 through 43 and was noted to have stiff rear legs from D44 through 49. As this animal was in the strict control group, the clinical signs were unrelated to vaccination or challenge. Three additional animals were observed to have sporadic clinical signs. Animal #14 (Group 1) was observed to have pronounced ribs and backbone and was not eating (body condition score of 2) shortly after arrival at AMVC on D13. In addition, the animal was noted to have a rough hair coat on D16 and 17. As clinical signs started prior to challenge and were not present until 13 days following vaccination, the signs are thought to be associated with movement of the CDCD animal at a young age not vaccination or challenge. Animal #11 (Group 1) was observed to have depression/lethargy (neurology score of 1) on D19. As this animal did not have evidence of viremia throughout the study, it is unlikely that the clinical signs were associated with challenge. Animal #5 (Group 9) was observed to be slightly thin compared to pen mates with a mild decrease in appetite (body condition score of 1) on D19. As transient viremia was detected in this animal on D15, the clinical sign may have been associated with infection. However, the clinical signs were not consistent with a previous publication and were transient.

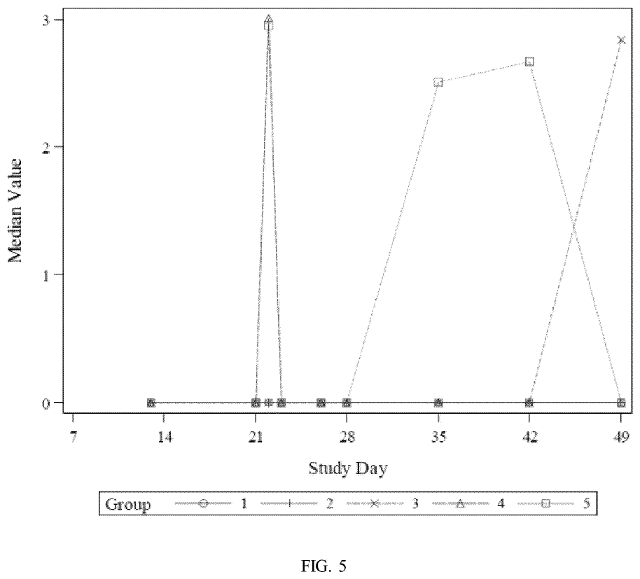

Fecal shedding was not detected in any of the six strict control animals throughout the study (Group 6). Frequency distributions of fecal shedding by group are presented in Table 10. Group median log 10 PCV3 DNA genomic copies/mL in fecal samples by day for Groups 1-5 are presented in FIG. 5.

TABLE 10
Frequency of PCV3 DNA detection by group in fecal samples

| | | Fecal shedding | | | |
|---|---|---|---|---|---|
| | | detected (ever) | | | |
| Group | Treatment | No | Yes | Total | % positive |
| 1 | BaculoPCV3/ISA-WV | 7 | 1 | 8 | 12.5% |
| 2 | BaculoPCV3/Carb-WV | 5 | 3 | 8 | 37.5% |
| 3 | Placebo/Carb-WV | 1 | 7 | 8 | 57.5% |
| 4 | BaculoPCV3/Carb-TH | 1 | 11 | 12 | 91.7% |
| 5 | Placebo/Carb-TH | 0 | 12 | 12 | 100.0% |
| 6 | Strict control | 6 | 0 | 6 | 0.0% |

In non-vaccinated pigs, exposure to the whole virus challenge material resulted in shedding in 88% of animals (Groups 3). Fecal shedding in these animals was first observed between D35 and D49. In contrast, fecal shedding was prevented in 75% (12/16) of vaccinated animals exposed to the whole virus challenge (p=0.0101 (Group 1 vs 3); p=0.1189 (Group 2 vs 3)). Overall, shedding in the vaccinated animals was sporadic and appeared inconsistent with a true infection.

In non-vaccinated pigs, exposure to the tissue homogenate challenge material resulted in fecal shedding in 100% of animals (Group 5). Fecal shedding in these animals was biphasic with multiple animals have detectable amounts of PCV3 in the feces on D22 and again on D35-49. Fecal shedding was observed in 92% of vaccinated animals. However, unlike non-vaccinated animals, shedding was most prevalent on D22 and D23 without a second peak.

PCV3 DNA was not detected in any of the fecal samples collected from animals in Groups 7-9.

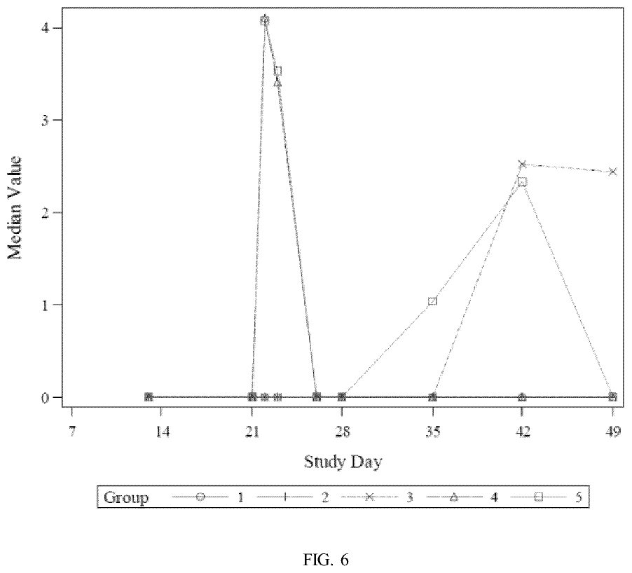

Nasal shedding was not detected in any of the strict control animals throughout the study (Group 6) with the exception of animal #59. As PCV3 DNA was only detected on D15 and all other samples (serum, fecal) were negative, this is likely a false positive. Group median log 10 PCV3 DNA genomic copies/mL in fecal samples by day for Groups 1-5 are presented in FIG. 6. (*Nasal detection in animal #59 is thought to be a false positive.)

In non-vaccinated pigs, exposure to the whole virus challenge material resulted in nasal shedding in 88% of animals (Group 3). Nasal shedding in these animals was first observed between D35 and D49. In contrast, nasal shedding was prevented in 94% (15/16) of vaccinated animals exposed to the whole virus challenge (p=0.0014 (Group 1 vs 3); p=0.0101 (Group 2 vs 3)). The one vaccinated animal (#19, Group 2) considered positive had PCV2 detection on D49 only.

In non-vaccinated pigs, exposure to the tissue homogenate challenge material resulted in nasal shedding in 100% of animals (Group 5). Nasal shedding in these animals was biphasic with multiple animals having detectable amounts of PCV3 in the nares on D22 and again on D35-49. Nasal shedding was observed in 100% of vaccinated animals. However, unlike non-vaccinated animals, nasal shedding was present in the majority of animals on D22 and 23 without a second peak. Sporadic shedding was seen in only two animals after D28.

Only two animals per group were included in the infectious clone portion of the study, the raw data by animal and day is presented in Table 11. PCV3 DNA was detected 5/6 animals the day after inoculation (D15) and in all animals regardless of the inoculum between D16-21. Only one animal (#4; Group 8) had detectable PCV3 DNA in nasal swabs after D21.

TABLE 11
Log10 PCV3 DNA genomic copies/mL in nasal swabs by animal and day for Groups 7-9

| Group | Treatment | Animal | D13 | D15 | D16 | D19 | D21 | D23 | D28 |
|---|---|---|---|---|---|---|---|---|---|
| 7 | IC-BI AH USA | 1 | 0.00 | 5.66 | 4.29 | 3.95 | 3.22 | 0.00 | 0.00 |
| | re-circularized | 2 | 0.00 | 4.82 | 5.18 | 4.18 | 3.77 | 0.00 | 0.00 |
| | genome | | | | | | | | |
| 8 | IC-ISU VDL | 3 | 0.00 | 3.56 | 4.65 | 3.01 | 2.44 | 0.00 | 0.00 |
| | dimerized | 4 | 0.00 | 4.70 | 3.58 | 3.73 | 2.34 | 0.00 | 2.23 |
| | genome | | | | | | | | |
| 9 | IC-ISU VDL | 5 | 0.00 | 2.82 | 3.48 | 3.27 | 2.55 | 0.00 | 0.00 |
| | transfection | 6 | 0.00 | 0.00 | 3.91 | 3.33 | 3.58 | 0.00 | 0.00 |
| | harvest | | | | | | | | |

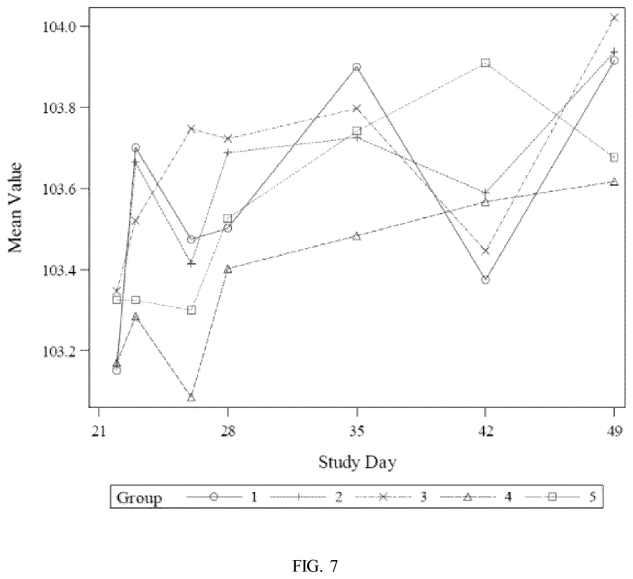

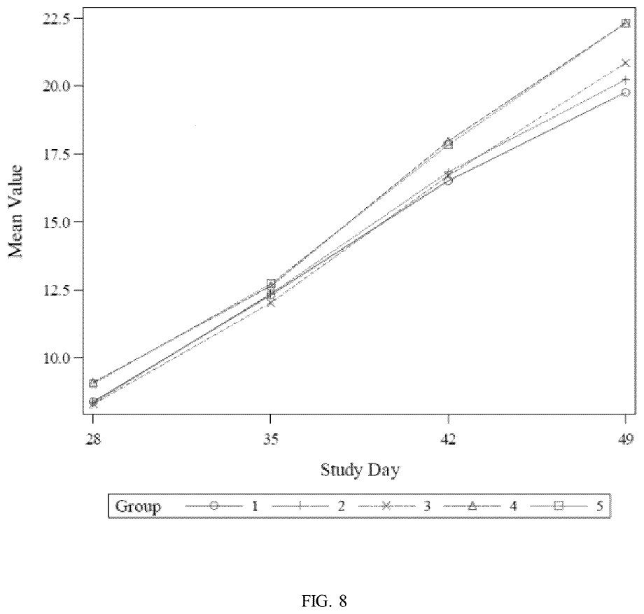

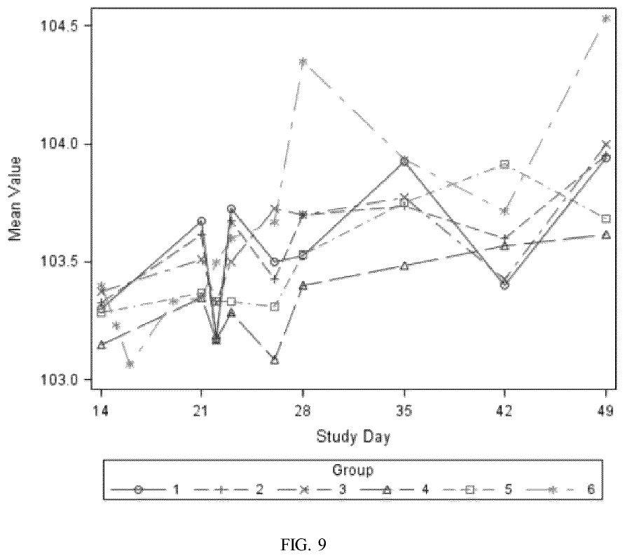

Baseline adjusted, least square group mean rectal temperatures (° F.) by study day are presented in FIG. 7. Data for Group 6 is not included in the figure as the analysis was model-based and animals in Group 6 were housed in a separate room. Raw data and descriptive statistics by group and day can be found in the statistical report associated with this study. No differences were observed between Groups 1-3 regardless of the vaccination status. Vaccinated animals challenged with the tissue homogenate (Group 4) had significantly lower temperatures in comparison to non-vaccinated challenged animals (Group 5) during the challenge period (p=0.0021).

No animal was considered pyrexic (had a temperature greater than 104° F.) throughout the study.

Baseline-adjusted, group least square means weights (kg) are presented for Groups 1-5 in FIG. 7. Raw data and descriptive statistics for all groups can be found in the statistical report associated with this study. No differences were observed between groups regardless of the challenge material or vaccination status (p>0.1).

The first objective of this study was to develop a challenge model for PCV3 in CDCD pigs and define the primary and secondary outcome variables. Two challenge materials, tissue homogenate and a whole virus were evaluated. As 100% of animals exposed to the tissue homogenate became viremic within 24 hours of challenge and had detectable nasal and fecal shedding, the material was considered highly infectious. The development of PCV3 viremia and shedding by fecal and nasal routes did not appear to require a co-infection as other pathogens, including PRRSV, PCV2 and PPV, were not detected by routine culture, deep sequencing, and specific PCR assays conducted on the original tissues.

The whole virus material resulted in viremia in 100% and nasal and fecal shedding in 88% of animals and is therefore considered infectious. However, viremia occurred 14 days following challenge; considerably slower in comparison to the tissue homogenate. The hypothesis is that the delay is related to the viral load of the challenge material. Specifically, the Cq values of the tissue homogenate and the whole virus were 14.82 and 23.58, respectively, suggesting that the tissue homogenate contained a higher amount of PCV3 DNA in comparison to the whole virus.

As original PCV3 case reports in the field were of reproductive failure and PDNS in sows [3], it was hypothesized that infection of CDCD pigs may result in PDNS. However, there was no outward evidence of PDNS (or other clinical disease) or pyrexia following exposure to either of the challenge materials. Because the tissue homogenate contained a high amount of virus and the onset of viremia was within 24 hours, it is unlikely that infection of CDCD pigs with PCV3 alone will result in PDNS. Therefore, based on the currently available data, viremia appears to be the most suitable primary parameter for use in future studies using the CDCD pig model. Also, fecal and nasal shedding were each reduced and could be used as secondary parameters. Biologically significant differences were not observed in body temperatures or weights; these parameters are not likely useful for future studies. As other parameters (serology, histopathology) were not evaluated at the time of the report generation, these may provide additional parameters.

The first objective of the study incorporated the initial evaluation of a vaccine prototype using two different adjuvants. This study provides preliminary data that one intramuscular dose of a baculovirus-expressed PCV3 ORF2 antigen administered to three week old pigs prevented viremia, nasal shedding, and fecal shedding following challenge with whole virus. Little to no shedding or viremia was detected in the animals of Groups 1 and 2, therefore, a strong conclusion to the preference of one adjuvant over the other cannot be made. The data from Group 3 and 4 suggest that the efficacy of the vaccine is reduced when the challenge material contains higher amounts of PCV3 DNA. Therefore, establishing a challenge dose which results in infection but will not overwhelm vaccination can be useful for future efficacy studies.

In order to evaluate the efficacy of PCV3 vaccination in a singular co-infection model, the CDCD pigs were vaccinated at seven days of age against PCV2. Based on the differences in capsid amino acid structure (26% amino acid identity in the cap gene between the two viruses [2]) it was hypothesized that there would be no cross-protection. Based on the results of this study, PCV2 vaccination did not appear to prevent PCV3 viremia, therefore, it is unlikely that PCV2 vaccination had any role in the lack of clinical disease.

The second objective of this study was to confirm the infectivity of infectious molecular clones generated by an external collaborator and an internal molecular clone generated by the vaccine design group. Interestingly, intrahepatic inoculation of the CDCD pigs with the infectious clone materials resulted in detectable nasal shedding for seven days following challenge. It is hypothesized that a transient viremia led to distribution of the virus to the nasal epithelium where replication occurred. Further studies and evaluation of nasal tissue with an antigen specific reagent will be needed to confirm this hypothesis. It is unknown why viremia was detected again in animal #3 on D28 through 49. Perhaps if larger numbers of animals had been used, detection of viremia would have occurred in a larger percentage of animals. While the development of viremia for multiple weeks suggests that animal #3 truly became infected, the infection was subclinical. This result does not agree with a recent publication [25] in which infection of conventional four week old pigs with a PCV3 infectious clone resulted in PDNS.

One intramuscular dose of a baculovirus-expressed PCV3 ORF2 antigen administered to three week old pigs prevented viremia, nasal shedding, and fecal shedding following challenge with tissue homogenate challenge material, which was considered infectious. In research studies or reasonable expectation of efficacy studies, viremia can be used as a primary parameter for vaccination evaluation. For future pivotal studies associated with a fully licensed product, a different primary parameter (detection of PCV3 antigen within tissues or clinical disease) would be required. Inoculation of CDCD pigs with infectious clone material resulted in viremia in one animal and nasal shedding in multiple animals. However, no clinical signs were observed.

### Example 4

### Vaccine Administered to Group 1

The vaccine designated as “Porcine Circovirus Vaccine, Type 3, Modified Live Baculovirus Vector” was by the following procedure. A 1 L lot of antigen was produced in a 3 L spinner flask by infecting SF+ (Spodoptera frugiperda) cells at an approximate MOI of 0.076 with a recombinant baculovirus containing the Porcine Circovirus 3 ORF2 gene under control of the baculovirus polyhedrin promoter. The flask was incubated at 28° C.±2° C. with constant agitation at approximately 100 rpm for seven days. Cells and media were aseptically transferred to 2×1 L centrifuge bottles and cells were pelleted at 15,000×g for 20 minutes at 4° C. The resulting supernatant was 0.2 μm filtered and stored at 4° C. The material was formulated with 50% ISA207 VG as shown Table 12. The vaccine satisfactorily completed sterility testing post-dispensation into final containers. Mouse safety was not conducted prior to putting the material into swine.

TABLE 12
Vaccine formulation-ISA207 VG adjuvant

| Component | Purpose | Lot no. | Weight | Concentration |
|---|---|---|---|---|
| BaculoG/PCV3 | Antigen | 3624-144 | 101.07 g | 50% |
| ORF2 | | | | |
| ISA2017 VG | Adjuvant | 15060911879 | 101.07 g | 50% |

### Example 5

### Vaccine Administered to Groups 2 and 4

Vaccine administered to Groups 2 and 4: Methods of Production—The vaccine designated as “Porcine Circovirus Vaccine, Type 3, Modified Live Baculovirus Vector” was produced by the method described as above for Group 1. Supernatant was formulated with 20% Carbopol as shown in Table 13. The vaccine satisfactorily completed sterility testing post-dispensation into final containers. Mouse safety was not conducted prior to putting the material into swine.

TABLE 13
Vaccine formulation-Carbopol adjuvant

| Component | Purpose | Lot no. | Volume |
|---|---|---|---|
| BaculoG/PCV3 | Antigen | 3624-144 | 80 mL |
| ORF2 | | | |
| Carbopol | Adjuvant | A80371 | 20 mL |

### Example 6

### Vaccine Administered to Groups 3 and 5

The vaccine designated as “Modified Live Baculovirus Vector” is a product-matched placebo. It was prepared by the following procedure. A 0.5 L lot of antigen was produced in a 1 L spinner flask by infecting SF+ (Spodoptera frugiperda) cells at an approximate MOI of 0.1 with a recombinant baculovirus containing no insert. The flask was incubated at 28° C.±2° C. with constant agitation at approximately 100 rpm for four days. Cells and media were aseptically transferred to a 1 L centrifuge bottle and cells were pelleted at 10,000×g for 20 minutes at 4° C. The resulting supernatant was 0.2 μm filtered and stored at 4° C. The material was formulated with 20% Carbopol as shown in Table 14. The vaccine satisfactorily completed sterility testing post-dispensation into final containers. Mouse Safety was not conducted prior to putting the material into swine.

TABLE 14
Placebo formulation-Carbopol adjuvant

| Component | Purpose | Lot no. | Volume |
|---|---|---|---|
| BaculoG/No | Antigen | 3624-153 | 60 mL |
| Insert control | | | |
| Carbopol | Adjuvant | A80371 | 15 mL |

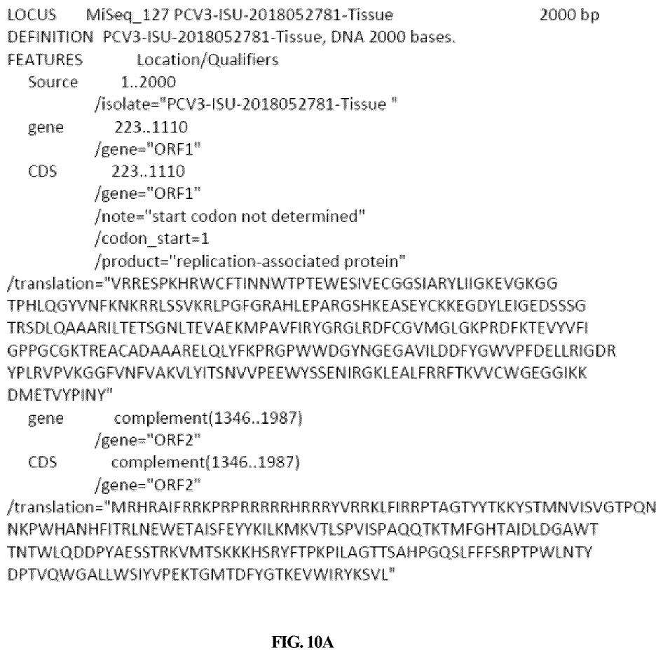

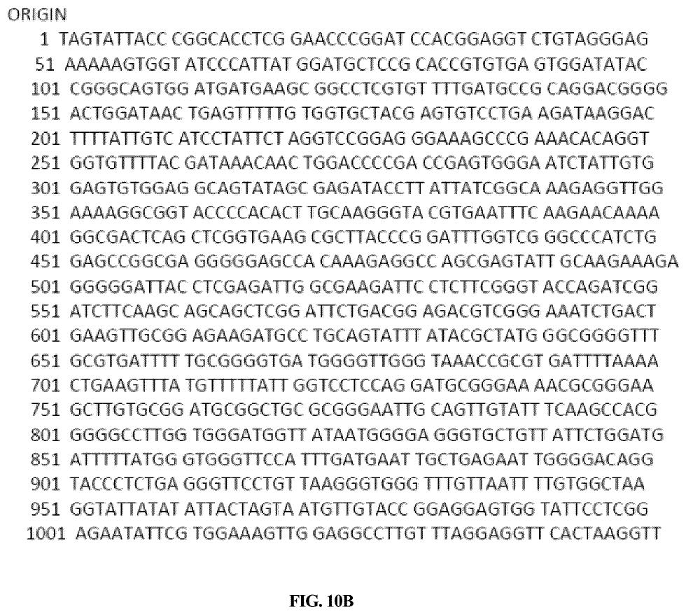

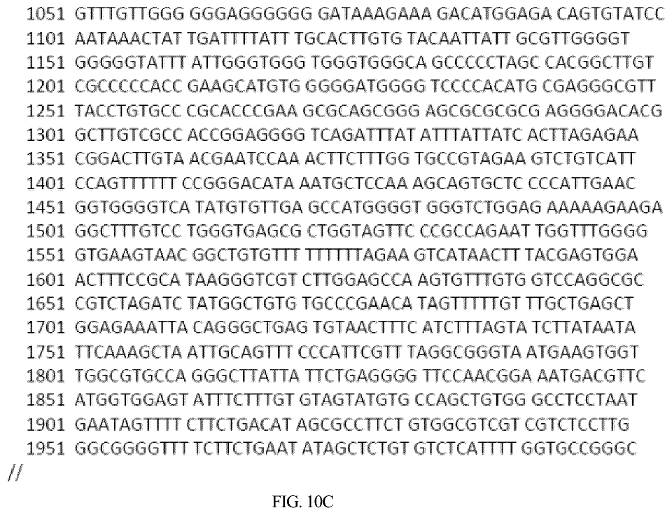

FIG. 10 shows sequence information on the PCV3 PCR positive tissue homogenate used for challenge material.

The pCR-BluntII-TOPO-PCV3 infectious clone plasmid was created from a 2,000 base pair PCV3 genome (KT869077) gBlock ordered from Integrated DNA Technologies (IDT). The gBlock was ligated into the pCR-BluntII-TOPO vector and transformed into Stb12 E. coli. The infectious clone plasmid was amplified and purified from a 1 L expansion of Stb12 E. coli using a Qiagen CompactPrep Maxi-DNA Purification kit following the manufacturer's recommended procedure. The pCR-BluntII-TOPO-PCV3 Clone 3624-046.06 Lot #3718-038 was diluted in sterile PBS pH7.4 Life Technologies Gibco Cat #10010-023 Lot #1967438 for a final concentration of 400 μg/mL of plasmid in a total of 4 mL. The diluted plasmid was aliquoted into a sterile vaccine bottle and stored at −20° C.

### Example 7

### Development of a PCV3 Challenge Model

PCV3 is an emerging disease in the global swine population and due to its potential correlation with clinical disease it has led to interest in the development of PCV3 vaccines. To evaluate prototype vaccines, the development of a challenge model was necessary.

As depicted in the following Tables and FIGS., Example 7 reflects studies conducted to develop a challenge model for PCV3 in pigs. In particular, caesarian derived, colostrum deprived (“CDCD”) pigs were used.

Studies were designed to evaluate the use of whole virus and PCR positive tissue homogenate as potential challenge materials for future studies. In addition, the rescue of a PCV3 infectious clone in pigs provided an additional option for future challenge model studies and was therefore incorporated into the study design.

Any prototype vaccines available during the course of experiments were included to provide a stronger evaluation of the challenge model.

PCV3 was isolated from clinical material. Virus isolation was confirmed by real-time qPCR transmission electron microscopy and immunofluorescence assay using suitable antibodies. The isolated viral harvest was shown to be free of other viruses including PCV1, PCV2, PRRSV, SIV, swine coronaviruses. Virus harvest provided was a pure culture. Purity was confirmed using Next Generation Sequencing.

The entire PCV3 genome was cloned into a suitable plasmid vector by full synthetic synthesis of the whole PCV3.

The genomic sequence was confirmed and the genome was cut out of the plasmid enzymatic digestion. The genome was then relegated to generate a closed covalent circular PCV3 genome.

The circularized PCV3 genome was transfected into suitable cell lines to rescue infectious virus. The rescued virus and/or circularized genome was inoculated into swine. Circularized genome was delivered into the liver and inguinal lymph node guided by ultrasound.

In a second iteration, plasmids were generated that contained two copies of the PCV3 genome. Sufficient quantities of purified plasmid containing the dimeric PCV3 were made for use in challenge model development and pathogenicity/virulence studies.

Clinical material, including tissue and fluids, containing high titer PCV3 as determined by qPCR were generated. The clinical material was shown to be free of other swine viruses including PCV1, PCV2, PRRSV, SIV, and/or swine coronaviruses.

Clinical material was used to develop a PCV3 challenge model and for pathogenicity/virulence studies. Animal studies were conducted to evaluate pathogenicity and spread of the virus using various routes of inoculation. Specifically, in addition to other routes being evaluated, PCV3 viral harvest and/or high titer tissue homogenate was inoculated into one horn of the uterus of sows at 40 days of gestation. PCV3 spread to the fetus in the inoculated horn and the non-inoculated uterine horn was evaluated. Development of mummies as a result of PCV3 infection was evaluated.

The challenge model was used to form the basis for evaluation of vaccine candidates.

Samples from PCV3 studies were tested, including pre-screen PCRs and serology, PCRs for the challenge model and infectious clones, serology for vaccine studies.

Limit of detection, sensitivity and specificity of assays were conducted.

Vaccine candidates were evaluated in different adjuvant combinations. Vaccine candidates included, for example, baculovirus expressed PCV3 ORF2 and PCV3 genome expressed in plasmid (nucleic acid vaccine). Serology was conducted for the vaccine study.

Table 15 relates to product dosing and how the animals were housed. In particular, Table 15 shows animals evaluated by groups. In particular, the litter, specific animal, whether they were vaccinated, the room they were in and the tub they were in were identified.

TABLE 15

| | | | Vaccinated | | |
|---|---|---|---|---|---|
| | | | 0 = no, | | |
| Group | Litter | Animal | 1 = yes | Room | Tub |
| 1 | 3 | 7 | 1 | A | 1 |
| | | 8 | 1 | A | 1 |
| | 4 | 9 | 1 | A | 2 |
| | | 10 | 1 | A | 2 |
| | 6 | 11 | 1 | A | 4 |
| | | 12 | 1 | A | 3 |
| | 10 | 13 | 1 | A | 5 |
| | | 14 | 1 | A | 4 |
| 2 | 3 | 15 | 1 | A | 1 |
| | | 16 | 1 | A | 1 |
| | 4 | 17 | 1 | A | 3 |
| | | 18 | 1 | A | 2 |
| | 6 | 19 | 1 | A | 3 |
| | | 20 | 1 | A | 4 |
| | 10 | 21 | 1 | A | 4 |
| | | 22 | 1 | A | 5 |
| 3 | 3 | 23 | 1 | A | 1 |
| | | 24 | 1 | A | 2 |
| | 4 | 25 | 1 | A | 3 |
| | | 26 | 1 | A | 2 |
| | 6 | 27 | 1 | A | 4 |
| | | 28 | 1 | A | 3 |
| 3 | 10 | 29 | 1 | A | 5 |
| | | 30 | 1 | A | 5 |
| 4 | 3 | 31 | 1 | B | 4 |
| | | 32 | 1 | B | 3 |
| | | 33 | 1 | B | 5 |
| | 4 | 34 | 1 | B | 3 |
| | | 35 | 1 | B | 5 |
| | | 36 | 1 | B | 2 |
| | 6 | 37 | 1 | B | 4 |
| | | 38 | 1 | B | 2 |
| | | 39 | 1 | B | 2 |
| | 10 | 40 | 1 | B | 1 |
| | | 41 | 1 | B | 4 |
| | | 42 | 1 | B | 1 |
| 5 | 3 | 43 | 1 | B | 3 |
| | | 44 | 1 | B | 5 |
| | | 45 | 1 | B | 4 |
| | 4 | 46 | 1 | B | 5 |
| | | 47 | 1 | B | 3 |
| | | 48 | 1 | B | 3 |
| | 6 | 49 | 1 | B | 2 |
| | | 50 | 1 | B | 5 |
| | | 51 | 1 | B | 2 |
| 5 | 10 | 52 | 1 | B | 1 |
| | | 53 | 1 | B | 4 |
| | | 54 | 1 | B | 1 |
| 6 | 3 | 55 | 0 | C | 2 |
| | | 56 | 0 | C | 1 |
| | 4 | 57 | 0 | C | 2 |
| | | 58 | 0 | C | 1 |
| | 6 | 59 | 0 | C | 1 |
| | | 60 | 0 | C | 2 |
| 7 | 5 | 1 | 0 | D | 1 |
| | 9 | 2 | 0 | D | 1 |
| 8 | 5 | 3 | 0 | D | 2 |
| | 9 | 4 | 0 | D | 2 |
| 9 | 5 | 5 | 0 | D | 3 |
| | 9 | 6 | 0 | D | 3 |

The following data relate to viremia data in animal subjects and the analysis thereof.

As is shown in Table 16, viremia values measured using qPCR Serum and shown in log genomic copies/mL are depicted by group for animals on a selection of study days.

TABLE 16

| | | Study Day | | | | | | | | | | | | |
|---|---|---|---|---|---|---|---|---|---|---|---|---|---|---|
| Group | Animal | 0 | 13 | 15 | 16 | 19 | 21 | 22 | 23 | 26 | 28 | 35 | 42 | 49 |
| 1 | 7 | 0.00 | 0.00 | — | — | — | 0.00 | 0.00 | 0.00 | 0.00 | 0.00 | 0.00 | 0.00 | 0.00 |
| | 8 | 0.00 | 0.00 | — | — | — | 0.00 | 0.00 | 0.00 | 0.00 | 0.00 | 0.00 | 0.00 | 0.00 |
| | 9 | 0.00 | 0.00 | — | — | — | 0.00 | 0.00 | 0.00 | 0.00 | 0.00 | 0.00 | 0.00 | 0.00 |
| | 10 | 0.00 | 0.00 | — | — | — | 0.00 | 0.00 | 0.00 | 0.00 | 0.00 | 0.00 | 0.00 | 0.00 |
| | 11 | 0.00 | 0.00 | — | — | — | 0.00 | 0.00 | 0.00 | 0.00 | 0.00 | 0.00 | 0.00 | 0.00 |
| | 12 | 0.00 | 0.00 | — | — | — | 0.00 | 0.00 | 0.00 | 0.00 | 0.00 | 0.00 | 0.00 | 0.00 |
| | 13 | 0.00 | 0.00 | — | — | — | 0.00 | 0.00 | 0.00 | 0.00 | 0.00 | 0.00 | 0.00 | — |
| | 14 | 0.00 | 0.00 | — | — | — | 0.00 | 0.00 | 0.00 | 0.00 | 0.00 | 0.00 | 0.00 | 4.05 |
| 2 | 15 | 0.00 | 0.00 | — | — | — | 0.00 | 0.00 | 0.00 | 0.00 | 0.00 | 0.00 | 0.00 | 0.00 |
| | 16 | 0.00 | 0.00 | — | — | — | 0.00 | 0.00 | 0.00 | 0.00 | 0.00 | 0.00 | 0.00 | 0.00 |
| | 17 | 0.00 | 0.00 | — | — | — | 0.00 | 0.00 | 0.00 | 0.00 | 0.00 | 0.00 | 0.00 | 0.00 |
| | 18 | 0.00 | 0.00 | — | — | — | 0.00 | 0.00 | 0.00 | 0.00 | 0.00 | 0.00 | 0.00 | 0.00 |
| | 19 | 0.00 | 0.00 | — | — | — | 0.00 | 0.00 | 0.00 | 0.00 | 0.00 | 0.00 | 0.00 | 0.00 |
| | 20 | 0.00 | 0.00 | — | — | — | 0.00 | 0.00 | 0.00 | 0.00 | 0.00 | 0.00 | 0.00 | 0.00 |
| | 21 | 0.00 | 0.00 | — | — | — | 0.00 | 0.00 | 0.00 | 0.00 | 0.00 | 0.00 | 0.00 | 0.00 |
| | 22 | 0.00 | 0.00 | — | — | — | 0.00 | 0.00 | 0.00 | 0.00 | 0.00 | 0.00 | 0.00 | 0.00 |
| 3 | 23 | 0.00 | 0.00 | — | — | — | 0.00 | 0.00 | 0.00 | 0.00 | 0.00 | 0.00 | 5.76 | 5.70 |
| | 24 | 0.00 | 0.00 | — | — | — | 0.00 | 0.00 | 0.00 | 0.00 | 0.00 | 5.05 | 0.00 | 6.25 |
| | 25 | 0.00 | 0.00 | — | — | — | 0.00 | 0.00 | 0.00 | 0.00 | 4.24 | 6.02 | 6.47 | 6.37 |
| | 26 | 0.00 | 0.00 | — | — | — | 0.00 | 0.00 | 0.00 | 0.00 | 0.00 | 3.78 | 6.71 | 5.72 |
| | 27 | 0.00 | 0.00 | — | — | — | 0.00 | 0.00 | 0.00 | 0.00 | 0.00 | 5.39 | 6.03 | 5.72 |
| | 28 | 0.00 | 0.00 | — | — | — | 0.00 | 0.00 | 0.00 | 0.00 | 0.00 | 5.29 | 7.04 | 6.93 |
| | 29 | 0.00 | 0.00 | — | — | — | 0.00 | 0.00 | 0.00 | 0.00 | 0.00 | 4.71 | 6.86 | 6.22 |
| | 30 | 0.00 | 0.00 | — | — | — | 0.00 | 0.00 | 0.00 | 0.00 | 0.00 | 4.64 | 6.50 | 5.80 |
| 4 | 31 | 0.00 | 0.00 | — | — | — | 0.00 | 0.00 | 0.00 | 0.00 | 0.00 | 0.00 | 0.00 | 0.00 |
| | 32 | 0.00 | 0.00 | — | — | — | 0.00 | 0.00 | 0.00 | 0.00 | 0.00 | 0.00 | 0.00 | 0.00 |
| | 33 | 0.00 | 0.00 | — | — | — | 0.00 | 0.00 | 0.00 | 0.00 | 0.00 | 0.00 | 0.00 | 0.00 |
| | 34 | 0.00 | 0.00 | — | — | — | 0.00 | 0.00 | 0.00 | 0.00 | 0.00 | 2.52 | 3.57 | 5.02 |
| | 35 | 0.00 | 0.00 | — | — | — | 0.00 | 0.00 | 0.00 | 0.00 | 0.00 | 0.00 | 0.00 | 3.44 |
| | 36 | 0.00 | 0.00 | — | — | — | 0.00 | 3.41 | 0.00 | 0.00 | 0.00 | 2.37 | 0.00 | 3.41 |
| | 37 | 0.00 | 0.00 | — | — | — | 0.00 | 0.00 | 0.00 | 0.00 | 0.00 | 0.00 | 0.00 | 0.00 |
| | 38 | 0.00 | 0.00 | — | — | — | 0.00 | 0.00 | 0.00 | 0.00 | 0.00 | 3.04 | 3.81 | 4.52 |
| | 39 | 0.00 | 0.00 | — | — | — | 0.00 | 0.00 | 0.00 | 0.00 | 0.00 | 0.00 | 3.67 | 3.55 |
| | 40 | 0.00 | 0.00 | — | — | — | 0.00 | 4.81 | 3.64 | 3.34 | 4.42 | 5.62 | 6.51 | 6.76 |
| | 41 | 0.00 | 0.00 | — | — | — | 0.00 | 0.00 | 0.00 | 0.00 | 0.00 | 0.00 | 0.00 | 0.00 |
| | 42 | 0.00 | 0.00 | — | — | — | 0.00 | 0.00 | 0.00 | 0.00 | 0.00 | 0.00 | 0.00 | 3.38 |
| 5 | 43 | 0.00 | 0.00 | — | — | — | 0.00 | 4.53 | 3.82 | 3.19 | 4.61 | 4.60 | 5.00 | 5.67 |
| | 44 | 0.00 | 0.00 | — | — | — | 0.00 | 4.50 | 3.16 | 3.03 | 4.91 | 5.40 | 6.09 | 5.86 |
| | 45 | 0.00 | 0.00 | — | — | — | 0.00 | 4.04 | 3.20 | 3.34 | 4.31 | 5.29 | 5.54 | 6.10 |
| | 46 | 0.00 | 0.00 | — | — | — | 0.00 | 3.94 | 2.64 | 0.00 | 3.92 | 4.91 | 5.16 | 6.01 |
| | 47 | 0.00 | 0.00 | — | — | — | 0.00 | 4.07 | 3.32 | 2.92 | 4.31 | 5.47 | 5.64 | 5.71 |
| | 48 | 0.00 | 0.00 | — | — | — | 0.00 | 4.48 | 3.09 | 2.73 | 4.79 | 5.88 | 5.77 | 6.15 |
| | 49 | 0.00 | 0.00 | — | — | — | 0.00 | 4.31 | 0.00 | 3.05 | 5.73 | 4.85 | 5.65 | 5.30 |
| | 50 | 0.00 | 0.00 | — | — | — | 0.00 | 4.77 | 3.59 | 3.21 | 5.42 | 6.62 | 6.35 | 5.66 |
| | 51 | 0.00 | 0.00 | — | — | — | 0.00 | 4.95 | 3.56 | 3.19 | 5.63 | 4.95 | 5.65 | 5.36 |
| | 52 | 0.00 | 0.00 | — | — | — | 0.00 | 4.73 | 3.47 | 3.21 | 4.52 | 5.79 | 5.89 | 5.60 |
| | 53 | 0.00 | 0.00 | — | — | — | 0.00 | 4.48 | 3.17 | 3.16 | 4.33 | 4.88 | 5.89 | 6.12 |
| | 54 | 0.00 | 0.00 | — | — | — | 0.00 | 4.62 | 3.63 | 2.78 | 4.63 | 5.88 | 6.10 | 5.72 |
| 6 | 55 | 0.00 | 0.00 | 0.00 | 0.00 | 0.00 | 0.00 | 0.00 | 0.00 | 0.00 | 0.00 | 0.00 | 0.00 | 0.00 |
| | 56 | 0.00 | 0.00 | 0.00 | 0.00 | 0.00 | 0.00 | 0.00 | 0.00 | 0.00 | 0.00 | 0.00 | 0.00 | 0.00 |
| | 57 | 0.00 | 0.00 | 0.00 | 0.00 | 0.00 | 0.00 | 0.00 | 0.00 | 0.00 | 0.00 | 0.00 | 0.00 | 0.00 |
| | 58 | 0.00 | 0.00 | 0.00 | 0.00 | 0.00 | 0.00 | 0.00 | 0.00 | 0.00 | 0.00 | 0.00 | 0.00 | 0.00 |
| | 59 | 0.00 | 0.00 | 0.00 | 0.00 | 0.00 | 0.00 | 0.00 | 0.00 | 0.00 | 0.00 | 0.00 | 0.00 | 0.00 |
| | 60 | 0.00 | 0.00 | 0.00 | 0.00 | 0.00 | 0.00 | 0.00 | 0.00 | 0.00 | 0.00 | 0.00 | 0.00 | 0.00 |
| 7 | 1 | 0.00 | 0.00 | 3.37 | 0.00 | 0.00 | 0.00 | — | 0.00 | — | 0.00 | 0.00 | 0.00 | 0.00 |
| | 2 | 0.00 | 0.00 | 3.84 | 2.58 | 0.00 | 0.00 | — | 0.00 | — | 0.00 | 0.00 | 0.00 | 0.00 |
| 8 | 3 | 0.00 | 0.00 | 2.87 | 0.00 | 0.00 | 0.00 | — | 0.00 | — | 3.88 | 6.03 | 6.51 | 5.93 |
| | 4 | 0.00 | 0.00 | 3.41 | 0.00 | 0.00 | 0.00 | — | 0.00 | — | 0.00 | 0.00 | 0.00 | 0.00 |
| 9 | 5 | 0.00 | 0.00 | 2.81 | 0.00 | 0.00 | 0.00 | — | 0.00 | — | 0.00 | 0.00 | 0.00 | 0.00 |
| | 6 | 0.00 | 0.00 | 0.00 | 0.00 | 0.00 | 0.00 | — | 0.00 | — | 0.00 | 0.00 | 0.00 | 0.00 |

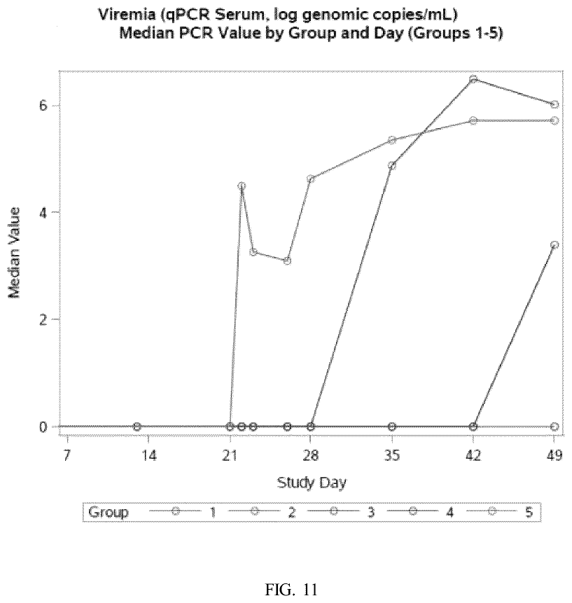

FIG. 11 shows the median PCR value for Groups 1-5 from seven to forty-nine days.

FIG. 12 shows the median PCR value for Groups 7-9 from seven to forty-nine.

Table 18 depicts results of viremia determinations for groups 1-5.

TABLE 18
Viremia (qPCR Serum, log genomic copies/mL: Viremia Results by Group
Table of grp by viremia

| | | viremia | | |
|---|---|---|---|---|
| | grp(Group) | No | Yes | Total |
| Frequency | 1 | 7 | 1 | 8 |
| Row Pct | | 87.50 | 12.50 | |
| | 2 | 8 | 0 | 8 |
| | | 100.00 | 0.00 | |
| | 3 | 0 | 8 | 8 |
| | | 0.00 | 100.00 | |
| | 4 | 5 | 7 | 12 |
| | | 41.67 | 58.33 | |
| | 5 | 0 | 12 | 12 |
| | | 0.00 | 100.00 | |
| | Total | 20 | 28 | 48 |

A comparison of the P-values for the data of Table 18 is shown in Table 19.

TABLE 19
Viremia (qPCR Serum, log genomic copies/mL)
Group Comparison P-values

| Group Comparison | P-value |
|---|---|
| 1 vs 3 | 0.0014 |
| 2 vs 3 | 0.0002 |
| 4 vs 5 | 0.0373 |

The following data relate to fecal shedding data measured using qPCR fecal (i.e., log genomic copies/mL) in animal subjects and the analysis thereof.

As is shown in Table 20, fecal shedding values measured using qPCR Fecal and shown in log genomic copies/mL are depicted by group for animals on a selection of study days.

TABLE 20

| | | Study Day | | | | | | | | | | | |
|---|---|---|---|---|---|---|---|---|---|---|---|---|---|
| Group | Animal | 13 | 15 | 16 | 19 | 21 | 22 | 23 | 26 | 28 | 35 | 42 | 49 |
| 1 | 7 | 0.00 | — | — | — | 0.00 | 0.00 | 0.00 | 0.00 | 0.00 | 2.88 | 0.00 | 0.00 |
| | 8 | 0.00 | — | — | — | 0.00 | 0.00 | 0.00 | 0.00 | 0.00 | 0.00 | 0.00 | 0.00 |
| | 9 | 0.00 | — | — | — | 0.00 | 0.00 | 0.00 | 0.00 | 0.00 | 0.00 | 0.00 | 0.00 |
| | 10 | 0.00 | — | — | — | 0.00 | 0.00 | 0.00 | 0.00 | 0.00 | 0.00 | 0.00 | 0.00 |
| | 11 | 0.00 | — | — | — | 0.00 | 0.00 | 0.00 | 0.00 | 0.00 | 0.00 | 0.00 | 0.00 |
| | 12 | 0.00 | — | — | — | 0.00 | 0.00 | 0.00 | 0.00 | 0.00 | 0.00 | 0.00 | 0.00 |
| | 13 | 0.00 | — | — | — | 0.00 | 0.00 | 0.00 | 0.00 | 0.00 | 0.00 | 0.00 | — |
| | 14 | 0.00 | — | — | — | 0.00 | 0.00 | 0.00 | 0.00 | 0.00 | 0.00 | 0.00 | 0.00 |
| 2 | 15 | 0.00 | — | — | — | 0.00 | 0.00 | 0.00 | 0.00 | 0.00 | 0.00 | 0.00 | 0.00 |
| | 16 | 0.00 | — | — | — | 0.00 | 0.00 | 0.00 | 0.00 | 0.00 | 0.00 | 0.00 | 0.00 |
| | 17 | 0.00 | — | — | — | 0.00 | 0.00 | 0.00 | 0.00 | 0.00 | 3.60 | 0.00 | 0.00 |
| | 18 | 0.00 | — | — | — | 0.00 | 0.00 | 0.00 | 3.99 | 0.00 | 3.42 | 0.00 | 0.00 |
| | 19 | 0.00 | — | — | — | 0.00 | 0.00 | 0.00 | 0.00 | 0.00 | 0.00 | 2.63 | 0.00 |
| | 20 | 0.00 | — | — | — | 0.00 | 0.00 | 0.00 | 0.00 | 0.00 | 0.00 | 0.00 | 0.00 |
| | 21 | 0.00 | — | — | — | 0.00 | 0.00 | 0.00 | 0.00 | 0.00 | 0.00 | 0.00 | 0.00 |
| | 22 | 0.00 | — | — | — | 0.00 | 0.00 | 0.00 | 0.00 | 0.00 | 0.00 | 0.00 | 0.00 |
| 3 | 23 | 0.00 | — | — | — | 0.00 | 0.00 | 0.00 | 0.00 | 0.00 | 0.00 | 0.00 | 0.00 |
| | 24 | 0.00 | — | — | — | 0.00 | 0.00 | 0.00 | 0.00 | 0.00 | 0.00 | 0.00 | 2.90 |
| | 25 | 0.00 | — | — | — | 0.00 | 0.00 | 0.00 | 0.00 | 0.00 | 3.46 | 4.11 | 4.32 |
| | 26 | 0.00 | — | — | — | 0.00 | 0.00 | 0.00 | 0.00 | 0.00 | 0.00 | 0.00 | 2.31 |
| | 27 | 0.00 | — | — | — | 0.00 | 0.00 | 0.00 | 0.00 | 0.00 | 2.53 | 0.00 | 0.00 |
| | 28 | 0.00 | — | — | — | 0.00 | 0.00 | 0.00 | 0.00 | 0.00 | 0.00 | 3.59 | 4.13 |
| | 29 | 0.00 | — | — | — | 0.00 | 0.00 | 0.00 | 0.00 | 0.00 | 0.00 | 3.17 | 3.46 |
| | 30 | 0.00 | — | — | — | 0.00 | 0.00 | 0.00 | 0.00 | 0.00 | 0.00 | 0.00 | 2.79 |
| 4 | 31 | 0.00 | — | — | — | 0.00 | 2.95 | 3.05 | 0.00 | 0.00 | 0.00 | 0.00 | 0.00 |
| | 32 | 0.00 | — | — | — | 0.00 | 3.33 | 3.09 | 0.00 | 0.00 | 0.00 | 0.00 | 0.00 |
| | 33 | 0.00 | — | — | — | 0.00 | 2.52 | 0.00 | 0.00 | 0.00 | 0.00 | 0.00 | 0.00 |
| | 34 | 0.00 | — | — | — | 0.00 | 2.44 | 0.00 | 0.00 | 0.00 | 0.00 | 0.00 | 0.00 |
| | 35 | 0.00 | — | — | — | 0.00 | 2.48 | 0.00 | 0.00 | 0.00 | 0.00 | 0.00 | 0.00 |
| | 36 | 0.00 | — | — | — | 0.00 | 3.06 | 0.00 | 0.00 | 0.00 | 0.00 | 0.00 | 0.00 |
| | 37 | 0.00 | — | — | — | 0.00 | 0.00 | 0.00 | 0.00 | 0.00 | 0.00 | 0.00 | 0.00 |
| | 38 | 0.00 | — | — | — | 0.00 | 3.37 | 2.90 | 0.00 | 0.00 | 0.00 | 0.00 | 0.00 |
| | 39 | 0.00 | — | — | — | 0.00 | 3.45 | 2.92 | 0.00 | 0.00 | 0.00 | 0.00 | 0.00 |
| | 40 | 0.00 | — | — | — | 0.00 | 3.55 | 3.49 | 0.00 | 0.00 | 2.75 | 2.54 | 2.62 |
| | 41 | 0.00 | — | — | — | 0.00 | 3.30 | 0.00 | 0.00 | 0.00 | 0.00 | 0.00 | 0.00 |
| | 42 | 0.00 | — | — | — | 0.00 | 2.67 | 0.00 | 0.00 | 0.00 | 0.00 | 0.00 | 0.00 |
| 5 | 43 | 0.00 | — | — | — | 0.00 | 3.24 | 0.00 | 0.00 | 0.00 | 0.00 | 0.00 | 0.00 |
| | 44 | 0.00 | — | — | — | 0.00 | 0.00 | 3.37 | 0.00 | 0.00 | 0.00 | 0.00 | 2.74 |
| | 45 | 0.00 | — | — | — | 0.00 | 0.00 | 0.00 | 0.00 | 0.00 | 0.00 | 2.68 | 2.36 |
| | 46 | 0.00 | — | — | — | 0.00 | 2.96 | 0.00 | 0.00 | 0.00 | 0.00 | 0.00 | 0.00 |
| | 47 | 0.00 | — | — | — | 0.00 | 2.94 | 0.00 | 0.00 | 0.00 | 2.49 | 3.37 | 2.55 |
| | 48 | 0.00 | — | — | — | 0.00 | 2.05 | 0.00 | 0.00 | 0.00 | 3.61 | 0.00 | 0.00 |
| | 49 | 0.00 | — | — | — | 0.00 | 0.00 | 0.00 | 0.00 | 0.00 | 2.67 | 4.39 | 0.00 |
| | 50 | 0.00 | — | — | — | 0.00 | 0.00 | 0.00 | 0.00 | 0.00 | 4.22 | 3.27 | 2.47 |
| | 51 | 0.00 | — | — | — | 0.00 | 3.37 | 0.00 | 0.00 | 0.00 | 2.53 | 2.70 | 0.00 |
| | 52 | 0.00 | — | — | — | 0.00 | 3.29 | 0.00 | 0.00 | 0.00 | 3.02 | 2.66 | 0.00 |
| | 53 | 0.00 | — | — | — | 0.00 | 3.28 | 0.00 | 0.00 | 0.00 | 0.00 | 0.00 | 0.00 |
| | 54 | 0.00 | — | — | — | 0.00 | 3.26 | 0.00 | 0.00 | 0.00 | 2.97 | 2.71 | 0.00 |
| 6 | 55 | 0.00 | 0.00 | 0.00 | 0.00 | 0.00 | 0.00 | 0.00 | 0.00 | 0.00 | 0.00 | 0.00 | 0.00 |
| | 56 | 0.00 | 0.00 | 0.00 | 0.00 | 0.00 | 0.00 | 0.00 | 0.00 | 0.00 | 0.00 | 0.00 | 0.00 |
| | 57 | 0.00 | 0.00 | 0.00 | 0.00 | 0.00 | 0.00 | 0.00 | 0.00 | 0.00 | 0.00 | 0.00 | 0.00 |
| | 58 | 0.00 | 0.00 | 0.00 | 0.00 | 0.00 | 0.00 | 0.00 | 0.00 | 0.00 | 0.00 | 0.00 | 0.00 |
| | 59 | 0.00 | 0.00 | 0.00 | 0.00 | 0.00 | 0.00 | 0.00 | 0.00 | 0.00 | 0.00 | 0.00 | 0.00 |
| | 60 | 0.00 | 0.00 | 0.00 | 0.00 | 0.00 | 0.00 | 0.00 | 0.00 | 0.00 | 0.00 | 0.00 | 0.00 |
| 7 | 1 | 0.00 | 0.00 | 0.00 | 0.00 | 0.00 | — | 0.00 | — | 0.00 | — | — | — |
| | 2 | 0.00 | 0.00 | 0.00 | 0.00 | 0.00 | — | 0.00 | — | 0.00 | — | — | — |
| 8 | 3 | 0.00 | 0.00 | 0.00 | 0.00 | 0.00 | — | 0.00 | — | 0.00 | — | — | — |
| | 4 | 0.00 | 0.00 | 0.00 | 0.00 | 0.00 | — | 0.00 | — | 0.00 | — | — | — |
| 9 | 5 | 0.00 | 0.00 | 0.00 | 0.00 | 0.00 | — | 0.00 | — | 0.00 | — | — | — |
| | 6 | 0.00 | 0.00 | 0.00 | 0.00 | 0.00 | — | 0.00 | — | 0.00 | — | — | — |

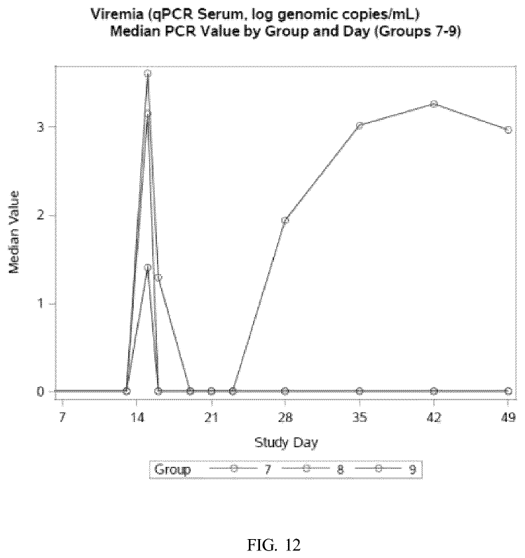

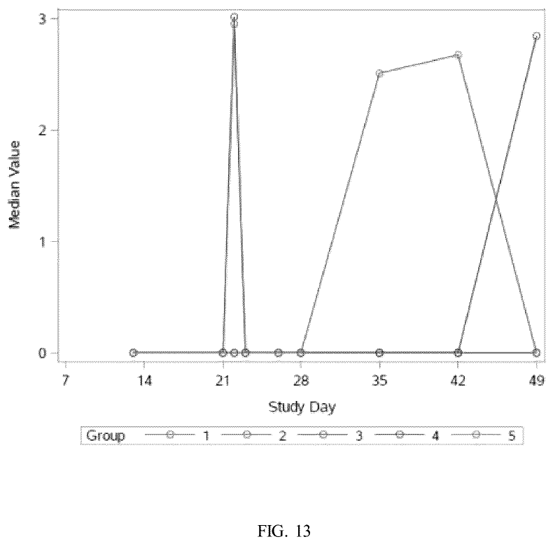

FIG. 13 shows the median PCR values for fecal shedding for Groups 1-5 from seven to forty-nine days.

Table 22 depicts results for fecal shedding determinations for groups 1-5.

TABLE 22
Table of grp by shedding

| | | shedding | | |
|---|---|---|---|---|
| | grp(Group) | No | Yes | Total |
| Frequency | 1 | 7 | 1 | 8 |
| Row Pct | | 87.50 | 12.50 | |
| | 2 | 5 | 3 | 8 |
| | | 62.50 | 37.50 | |
| | 3 | 1 | 7 | 8 |
| | | 12.50 | 87.50 | |
| | 4 | 1 | 11 | 12 |
| | | 8.33 | 91.67 | |
| | 5 | 0 | 12 | 12 |
| | | 0.00 | 100.00 | |
| | Total | 14 | 34 | 48 |

A comparison of the P-values for the data of Table 22 (fecal shedding determinations) is shown in Table 23.

TABLE 23

| Group Comparison | P-value |
|---|---|
| 1 vs 3 | 0.0101 |
| 2 vs 3 | 0.1169 |
| 4 vs 5 | 1.0000 |

A direct comparison of the P-values (i.e., Wilcoxon Test) for Group 4 and 5 is shown in Table 24.

TABLE 24

| Day | P-value |
|---|---|
| 22 | 0.239 |
| 23 | 0.131 |
| 26 | 1.000 |
| 28 | 1.000 |
| 35 | 0.014 |
| 42 | 0.005 |
| 49 | 0.261 |

The following data relate to nasal shedding data measured using qPCR Nasal (i.e., log genomic copies/mL) in animal subjects and the analysis thereof.

As is shown in Table 25, nasal shedding values measured using qPCR Nasal and shown in log genomic copies/mL are depicted by group for animals on a selection of study days.

TABLE 25

| | | Study Day | | | | | | | | | | | |
|---|---|---|---|---|---|---|---|---|---|---|---|---|---|
| Group | Animal | 13 | 15 | 16 | 19 | 21 | 22 | 23 | 26 | 28 | 35 | 42 | 49 |
| 1 | 7 | 0.00 | — | — | — | 0.00 | 0.00 | 0.00 | 0.00 | 0.00 | 0.00 | 0.00 | 0.00 |
| | 8 | 0.00 | — | — | — | 0.00 | 0.00 | 0.00 | 0.00 | 0.00 | 0.00 | 0.00 | 0.00 |
| | 9 | 0.00 | — | — | — | 0.00 | 0.00 | 0.00 | 0.00 | 0.00 | 0.00 | 0.00 | 0.00 |
| | 10 | 0.00 | — | — | — | 0.00 | 0.00 | 0.00 | 0.00 | 0.00 | 0.00 | 0.00 | 0.00 |
| | 11 | 0.00 | — | — | — | 0.00 | 0.00 | 0.00 | 0.00 | 0.00 | 0.00 | 0.00 | 0.00 |
| | 12 | 0.00 | — | — | — | 0.00 | 0.00 | 0.00 | 0.00 | 0.00 | 0.00 | 0.00 | 0.00 |
| | 13 | 0.00 | — | — | — | 0.00 | 0.00 | 0.00 | 0.00 | 0.00 | 0.00 | 0.00 | — |
| | 14 | 0.00 | — | — | — | 0.00 | 0.00 | 0.00 | 0.00 | 0.00 | 0.00 | 0.00 | 0.00 |
| 2 | 15 | 0.00 | — | — | — | 0.00 | 0.00 | 0.00 | 0.00 | 0.00 | 0.00 | 0.00 | 0.00 |
| | 16 | 0.00 | — | — | — | 0.00 | 0.00 | 0.00 | 0.00 | 0.00 | 0.00 | 0.00 | 0.00 |
| | 17 | 0.00 | — | — | — | 0.00 | 0.00 | 0.00 | 0.00 | 0.00 | 0.00 | 0.00 | 0.00 |
| | 18 | 0.00 | — | — | — | 0.00 | 0.00 | 0.00 | 0.00 | 0.00 | 0.00 | 0.00 | 0.00 |
| | 19 | 0.00 | — | — | — | 0.00 | 0.00 | 0.00 | 0.00 | 0.00 | 0.00 | 0.00 | 2.56 |
| | 20 | 0.00 | — | — | — | 0.00 | 0.00 | 0.00 | 0.00 | 0.00 | 0.00 | 0.00 | 0.00 |
| | 21 | 0.00 | — | — | — | 0.00 | 0.00 | 0.00 | 0.00 | 0.00 | 0.00 | 0.00 | 0.00 |
| | 22 | 0.00 | — | — | — | 0.00 | 0.00 | 0.00 | 0.00 | 0.00 | 0.00 | 0.00 | 0.00 |
| 3 | 23 | 0.00 | — | — | — | 0.00 | 0.00 | 0.00 | 0.00 | 0.00 | 0.00 | 0.00 | 0.00 |
| | 24 | 0.00 | — | — | — | 0.00 | 0.00 | 0.00 | 0.00 | 0.00 | 0.00 | 0.00 | 1.95 |
| | 25 | 0.00 | — | — | — | 0.00 | 0.00 | 0.00 | 0.00 | 0.00 | 2.24 | 2.72 | 1.95 |
| | 26 | 0.00 | — | — | — | 0.00 | 0.00 | 0.00 | 0.00 | 0.00 | 0.00 | 2.60 | 3.59 |
| | 27 | 0.00 | — | — | — | 0.00 | 0.00 | 0.00 | 0.00 | 0.00 | 0.00 | 2.25 | 1.97 |
| | 28 | 0.00 | — | — | — | 0.00 | 0.00 | 0.00 | 0.00 | 0.00 | 2.43 | 3.50 | 3.20 |
| | 29 | 0.00 | — | — | — | 0.00 | 0.00 | 0.00 | 0.00 | 0.00 | 0.00 | 2.67 | 3.85 |
| | 30 | 0.00 | — | — | — | 0.00 | 0.00 | 0.00 | 0.00 | 0.00 | 0.00 | 2.45 | 2.91 |
| 4 | 31 | 0.00 | — | — | — | 0.00 | 4.11 | 0.00 | 0.00 | 0.00 | 0.00 | 0.00 | 0.00 |
| | 32 | 0.00 | — | — | — | 0.00 | 4.70 | 3.68 | 0.00 | 0.00 | 0.00 | 0.00 | 0.00 |
| | 33 | 0.00 | — | — | — | 0.00 | 4.05 | 0.00 | 0.00 | 0.00 | 0.00 | 0.00 | 0.00 |
| | 34 | 0.00 | — | — | — | 0.00 | 4.07 | 0.00 | 0.00 | 0.00 | 0.00 | 0.00 | 0.00 |
| | 35 | 0.00 | — | — | — | 0.00 | 4.97 | 0.00 | 0.00 | 0.00 | 0.00 | 0.00 | 0.00 |
| | 36 | 0.00 | — | — | — | 0.00 | 3.68 | 3.50 | 0.00 | 0.00 | 0.00 | 0.00 | 0.00 |
| | 37 | 0.00 | — | — | — | 0.00 | 3.96 | 3.78 | 0.00 | 0.00 | 0.00 | 0.00 | 0.00 |
| | 38 | 0.00 | — | — | — | 0.00 | 4.48 | 0.00 | 0.00 | 0.00 | 0.00 | 0.00 | 0.00 |
| | 39 | 0.00 | — | — | — | 0.00 | 4.28 | 3.76 | 0.00 | 0.00 | 0.00 | 0.00 | 0.00 |
| | 40 | 0.00 | — | — | — | 0.00 | 4.10 | 3.85 | 0.00 | 0.00 | 0.00 | 2.33 | 2.01 |
| | 41 | 0.00 | — | — | — | 0.00 | 4.86 | 3.80 | 0.00 | 2.91 | 0.00 | 0.00 | 0.00 |
| | 42 | 0.00 | — | — | — | 0.00 | 0.00 | 3.33 | 0.00 | 0.00 | 0.00 | 0.00 | 0.00 |
| 5 | 43 | 0.00 | — | — | — | 0.00 | 0.00 | 4.30 | 0.00 | 0.00 | 0.00 | 0.00 | 0.00 |
| | 44 | 0.00 | — | — | — | 0.00 | 4.03 | 0.00 | 0.00 | 0.00 | 2.07 | 0.00 | 2.08 |
| | 45 | 0.00 | — | — | — | 0.00 | 4.01 | 3.62 | 0.00 | 0.00 | 0.00 | 2.60 | 0.00 |
| | 46 | 0.00 | — | — | — | 0.00 | 4.05 | 3.43 | 0.00 | 0.00 | 0.00 | 0.00 | 0.00 |
| | 47 | 0.00 | — | — | — | 0.00 | 4.11 | 0.00 | 0.00 | 0.00 | 0.00 | 3.13 | 2.10 |
| | 48 | 0.00 | — | — | — | 0.00 | 4.74 | 3.68 | 0.00 | 0.00 | 2.83 | 2.37 | 0.00 |
| | 49 | 0.00 | — | — | — | 0.00 | 3.92 | 0.00 | 0.00 | 0.00 | 2.44 | 0.00 | 0.00 |
| | 50 | 0.00 | — | — | — | 0.00 | 4.80 | 3.43 | 0.00 | 0.00 | 3.11 | 2.29 | 2.17 |
| | 51 | 0.00 | — | — | — | 0.00 | 5.08 | 3.58 | 0.00 | 0.00 | 2.26 | 2.71 | 0.00 |
| | 52 | 0.00 | — | — | — | 0.00 | 4.47 | 3.56 | 0.00 | 0.00 | 2.50 | 2.70 | 0.00 |
| | 53 | 0.00 | — | — | — | 0.00 | 4.64 | 3.97 | 0.00 | 0.00 | 0.00 | 2.25 | 0.00 |
| | 54 | 0.00 | — | — | — | 0.00 | 3.48 | 3.50 | 0.00 | 0.00 | 0.00 | 3.55 | 0.00 |
| 6 | 55 | 0.00 | 0.00 | 0.00 | 0.00 | 0.00 | 0.00 | 0.00 | 0.00 | 0.00 | 0.00 | 0.00 | 0.00 |
| | 56 | 0.00 | 0.00 | 0.00 | 0.00 | 0.00 | 0.00 | 0.00 | 0.00 | 0.00 | 0.00 | 0.00 | 0.00 |
| | 57 | 0.00 | 0.00 | 0.00 | 0.00 | 0.00 | 0.00 | 0.00 | 0.00 | 0.00 | 0.00 | 0.00 | 0.00 |
| | 58 | 0.00 | 0.00 | 0.00 | 0.00 | 0.00 | 0.00 | 0.00 | 0.00 | 0.00 | 0.00 | 0.00 | 0.00 |
| | 59 | 0.00 | 3.06 | 0.00 | 0.00 | 0.00 | 0.00 | 0.00 | 0.00 | 0.00 | 0.00 | 0.00 | 0.00 |
| | 60 | 0.00 | 0.00 | 0.00 | 0.00 | 0.00 | 0.00 | 0.00 | 0.00 | 0.00 | 0.00 | 0.00 | 0.00 |
| 7 | 1 | 0.00 | 5.66 | 4.29 | 3.95 | 3.22 | — | 0.00 | — | 0.00 | — | — | — |
| | 2 | 0.00 | 4.82 | 5.18 | 4.18 | 3.77 | — | 0.00 | — | 0.00 | — | — | — |
| 8 | 3 | 0.00 | 3.56 | 4.65 | 3.01 | 2.44 | — | 0.00 | — | 0.00 | — | — | — |
| | 4 | 0.00 | 4.70 | 3.58 | 3.73 | 2.34 | — | 0.00 | — | 2.23 | — | — | — |
| 9 | 5 | 0.00 | 2.82 | 3.48 | 3.27 | 2.55 | — | 0.00 | — | 0.00 | — | — | — |
| | 6 | 0.00 | 0.00 | 3.91 | 3.33 | 3.58 | — | 0.00 | — | 0.00 | — | — | — |

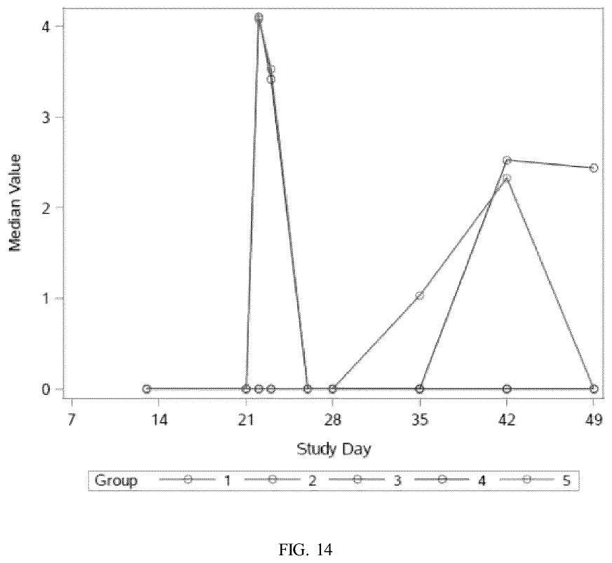

FIG. 14 shows the median PCR values for nasal shedding for Groups 1-5 from seven to forty-nine days.

Table 27 depicts results for nasal shedding determinations for groups 1-5.

TABLE 27
Table of grp by shedding

| | | shedding | | |
|---|---|---|---|---|
| | grp(Group) | No | Yes | Total |
| Frequency | 1 | 8 | 0 | 8 |
| Row Pct | | 100.00 | 0.00 | |
| | 2 | 7 | 1 | 8 |
| | | 87.50 | 12.50 | |
| | 3 | 1 | 7 | 8 |
| | | 12.50 | 87.50 | |
| | 4 | 0 | 12 | 12 |
| | | 0.00 | 100.00 | |
| | 5 | 0 | 12 | 12 |
| | | 0.00 | 100.00 | |
| | Total | 16 | 32 | 48 |

A comparison of the P-values for the data of Table 27 (nasal shedding determinations) is shown in Table 28.

TABLE 28

| Group Comparison | P-value |
|---|---|
| 1 vs 3 | 0.0014 |
| 2 vs 3 | 0.0101 |
| 4 vs 5 | 1.0000 |

A direct comparison of the P-values (i.e., Wilcoxon Test) for Group 4 and 5 is shown in Table 29.

TABLE 29

| Day | P-value |
|---|---|
| 22 | 0.812 |
| 23 | 0.760 |
| 26 | 1.000 |
| 28 | 1.000 |
| 35 | 0.014 |
| 42 | 0.003 |
| 49 | 0.217 |

The following data relate to rectal temperature (° F.) data measured in animal subjects and the analysis thereof.

As is shown in Table 30, rectal temperature values measured in Fahrenheit are depicted by group for animals on a number of study days.

TABLE 30

| | | Study Day | | | | | | | | | | | |
|---|---|---|---|---|---|---|---|---|---|---|---|---|---|
| Group | Animal | 14 | 15 | 16 | 19 | 21 | 22 | 23 | 26 | 28 | 38 | 42 | 49 |
| 1 | 7 | [symbol] | — | — | — | [symbol] | [symbol] | [symbol] | [symbol] | [symbol] | [symbol] | [symbol] | [symbol] |
| | 8 | [symbol] | — | — | — | [symbol] | [symbol] | [symbol] | [symbol] | [symbol] | [symbol] | [symbol] | [symbol] |
| | 9 | [symbol] | — | — | — | [symbol] | [symbol] | [symbol] | [symbol] | [symbol] | [symbol] | [symbol] | [symbol] |
| | 10 | [symbol] | — | — | — | [symbol] | [symbol] | [symbol] | [symbol] | [symbol] | [symbol] | [symbol] | [symbol] |
| | 11 | [symbol] | — | — | — | [symbol] | [symbol] | [symbol] | [symbol] | [symbol] | [symbol] | [symbol] | [symbol] |
| | 12 | [symbol] | — | — | — | [symbol] | [symbol] | [symbol] | [symbol] | [symbol] | [symbol] | [symbol] | [symbol] |
| | 13 | [symbol] | — | — | — | [symbol] | [symbol] | [symbol] | [symbol] | [symbol] | [symbol] | [symbol] | — |
| | 14 | [symbol] | — | — | — | [symbol] | [symbol] | [symbol] | [symbol] | [symbol] | [symbol] | [symbol] | [symbol] |
| 2 | 15 | [symbol] | — | — | — | [symbol] | [symbol] | [symbol] | [symbol] | [symbol] | [symbol] | [symbol] | [symbol] |
| | 16 | [symbol] | — | — | — | [symbol] | [symbol] | [symbol] | [symbol] | [symbol] | [symbol] | [symbol] | [symbol] |
| | 17 | [symbol] | — | — | — | [symbol] | [symbol] | [symbol] | [symbol] | [symbol] | [symbol] | [symbol] | [symbol] |
| | 18 | [symbol] | — | — | — | [symbol] | [symbol] | [symbol] | [symbol] | [symbol] | [symbol] | [symbol] | [symbol] |
| | 19 | [symbol] | — | — | — | [symbol] | [symbol] | [symbol] | [symbol] | [symbol] | [symbol] | [symbol] | [symbol] |
| | 20 | [symbol] | — | — | — | [symbol] | [symbol] | [symbol] | [symbol] | [symbol] | [symbol] | [symbol] | [symbol] |
| | 21 | [symbol] | — | — | — | [symbol] | [symbol] | [symbol] | [symbol] | [symbol] | [symbol] | [symbol] | [symbol] |
| | 22 | [symbol] | — | — | — | [symbol] | [symbol] | [symbol] | [symbol] | [symbol] | [symbol] | [symbol] | [symbol] |
| 3 | 23 | [symbol] | — | — | — | [symbol] | [symbol] | [symbol] | [symbol] | [symbol] | [symbol] | [symbol] | [symbol] |
| | 24 | [symbol] | — | — | — | [symbol] | [symbol] | [symbol] | [symbol] | [symbol] | [symbol] | [symbol] | [symbol] |
| | 25 | [symbol] | — | — | — | [symbol] | [symbol] | [symbol] | [symbol] | [symbol] | [symbol] | [symbol] | [symbol] |
| | 26 | [symbol] | — | — | — | [symbol] | [symbol] | [symbol] | [symbol] | [symbol] | [symbol] | [symbol] | [symbol] |
| | 27 | [symbol] | — | — | — | [symbol] | [symbol] | [symbol] | [symbol] | [symbol] | [symbol] | [symbol] | [symbol] |
| | 28 | [symbol] | — | — | — | [symbol] | [symbol] | [symbol] | [symbol] | [symbol] | [symbol] | [symbol] | [symbol] |
| | 29 | [symbol] | — | — | — | [symbol] | [symbol] | [symbol] | [symbol] | [symbol] | [symbol] | [symbol] | [symbol] |
| | 30 | [symbol] | — | — | — | [symbol] | [symbol] | [symbol] | [symbol] | [symbol] | [symbol] | [symbol] | [symbol] |
| 4 | 31 | [symbol] | — | — | — | [symbol] | [symbol] | [symbol] | [symbol] | [symbol] | [symbol] | [symbol] | [symbol] |
| | 32 | [symbol] | — | — | — | [symbol] | [symbol] | [symbol] | [symbol] | [symbol] | [symbol] | [symbol] | [symbol] |
| | 33 | [symbol] | — | — | — | [symbol] | [symbol] | [symbol] | [symbol] | [symbol] | [symbol] | [symbol] | [symbol] |
| | 34 | [symbol] | — | — | — | [symbol] | [symbol] | [symbol] | [symbol] | [symbol] | [symbol] | [symbol] | [symbol] |
| | 35 | [symbol] | — | — | — | [symbol] | [symbol] | [symbol] | [symbol] | [symbol] | [symbol] | [symbol] | [symbol] |
| | 36 | [symbol] | — | — | — | [symbol] | [symbol] | [symbol] | [symbol] | [symbol] | [symbol] | [symbol] | [symbol] |
| | 37 | [symbol] | — | — | — | [symbol] | [symbol] | [symbol] | [symbol] | [symbol] | [symbol] | [symbol] | [symbol] |
| | 38 | [symbol] | — | — | — | [symbol] | [symbol] | [symbol] | [symbol] | [symbol] | [symbol] | [symbol] | [symbol] |
| | 39 | [symbol] | — | — | — | [symbol] | [symbol] | [symbol] | [symbol] | [symbol] | [symbol] | [symbol] | [symbol] |
| | 40 | [symbol] | — | — | — | [symbol] | [symbol] | [symbol] | [symbol] | [symbol] | [symbol] | [symbol] | [symbol] |
| | 41 | [symbol] | — | — | — | [symbol] | [symbol] | [symbol] | [symbol] | [symbol] | [symbol] | [symbol] | [symbol] |
| | 42 | [symbol] | — | — | — | [symbol] | [symbol] | [symbol] | [symbol] | [symbol] | [symbol] | [symbol] | [symbol] |
| 5 | 43 | [symbol] | — | — | — | [symbol] | [symbol] | [symbol] | [symbol] | [symbol] | [symbol] | [symbol] | [symbol] |
| | 44 | [symbol] | — | — | — | [symbol] | [symbol] | [symbol] | [symbol] | [symbol] | [symbol] | [symbol] | [symbol] |
| | 45 | [symbol] | — | — | — | [symbol] | [symbol] | [symbol] | [symbol] | [symbol] | [symbol] | [symbol] | [symbol] |
| | 46 | [symbol] | — | — | — | [symbol] | [symbol] | [symbol] | [symbol] | [symbol] | [symbol] | [symbol] | [symbol] |
| | 47 | [symbol] | — | — | — | [symbol] | [symbol] | [symbol] | [symbol] | [symbol] | [symbol] | [symbol] | [symbol] |
| | 48 | [symbol] | — | — | — | [symbol] | [symbol] | [symbol] | [symbol] | [symbol] | [symbol] | [symbol] | [symbol] |
| | 49 | [symbol] | — | — | — | [symbol] | [symbol] | [symbol] | [symbol] | [symbol] | [symbol] | [symbol] | [symbol] |
| | 50 | [symbol] | — | — | — | [symbol] | [symbol] | [symbol] | [symbol] | [symbol] | [symbol] | [symbol] | [symbol] |
| | 51 | [symbol] | — | — | — | [symbol] | [symbol] | [symbol] | [symbol] | [symbol] | [symbol] | [symbol] | [symbol] |
| | 52 | [symbol] | — | — | — | [symbol] | [symbol] | [symbol] | [symbol] | [symbol] | [symbol] | [symbol] | [symbol] |
| | 53 | [symbol] | — | — | — | [symbol] | [symbol] | [symbol] | [symbol] | [symbol] | [symbol] | [symbol] | [symbol] |
| | 54 | [symbol] | — | — | — | [symbol] | [symbol] | [symbol] | [symbol] | [symbol] | [symbol] | [symbol] | [symbol] |
| 6 | 55 | [symbol] | [symbol] | [symbol] | [symbol] | [symbol] | [symbol] | [symbol] | [symbol] | [symbol] | [symbol] | [symbol] | [symbol] |
| | 56 | [symbol] | [symbol] | [symbol] | [symbol] | [symbol] | [symbol] | [symbol] | [symbol] | [symbol] | [symbol] | [symbol] | [symbol] |
| | 57 | [symbol] | [symbol] | [symbol] | [symbol] | [symbol] | [symbol] | [symbol] | [symbol] | [symbol] | [symbol] | [symbol] | [symbol] |
| | 58 | [symbol] | [symbol] | [symbol] | [symbol] | [symbol] | [symbol] | [symbol] | [symbol] | [symbol] | [symbol] | [symbol] | [symbol] |
| | 59 | [symbol] | [symbol] | [symbol] | [symbol] | [symbol] | [symbol] | [symbol] | [symbol] | [symbol] | [symbol] | [symbol] | [symbol] |
| | 60 | [symbol] | [symbol] | [symbol] | [symbol] | [symbol] | [symbol] | [symbol] | [symbol] | [symbol] | [symbol] | [symbol] | [symbol] |
| 7 | 1 | [symbol] | [symbol] | [symbol] | [symbol] | [symbol] | — | [symbol] | — | [symbol] | — | — | — |
| | 2 | [symbol] | [symbol] | [symbol] | [symbol] | [symbol] | — | [symbol] | — | [symbol] | — | — | — |
| 8 | 3 | [symbol] | [symbol] | [symbol] | [symbol] | [symbol] | — | [symbol] | — | [symbol] | — | — | — |
| | 4 | [symbol] | [symbol] | [symbol] | [symbol] | [symbol] | — | [symbol] | — | [symbol] | — | — | — |
| 9 | 5 | [symbol] | [symbol] | [symbol] | [symbol] | [symbol] | — | [symbol] | — | [symbol] | — | — | — |
| | 6 | [symbol] | [symbol] | [symbol] | [symbol] | [symbol] | — | [symbol] | — | [symbol] | — | — | — |

[symbol]

indicates data missing or illegible when filed

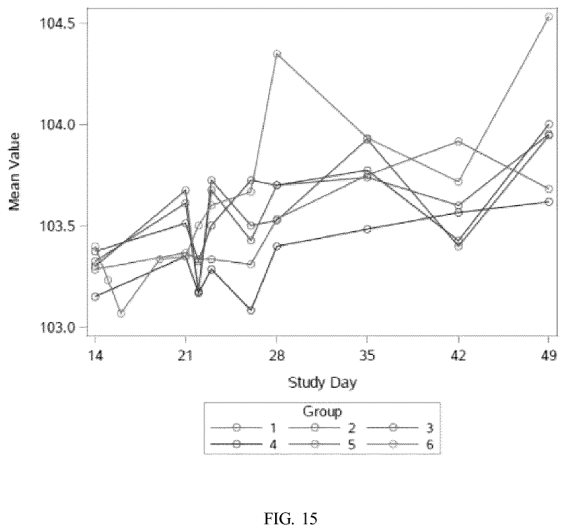

FIG. 15 depicts the arithmetic mean rectal temperature value for Groups 1-6 from fourteen to forty-nine days of the evaluation.

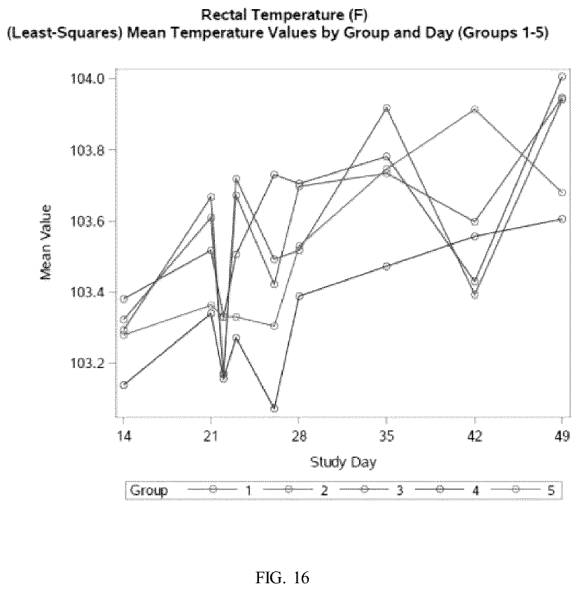

FIG. 16 depicts the least-squares mean temperature values by group and day for groups 1-5.

Table 53 shows a comparison of the P-values for the data relating to rectal temperature.

TABLE 53
Rectal Temperature (F.)
Group Comparison P-values

| Group Comparison | Day | estimate | P-value |
|---|---|---|---|
| 1 vs 2 | 14 | −0.03 | 0.8834 |
| | 21 | 0.06 | 0.7691 |
| | 22 | −0.00 | 0.9848 |
| | 23 | 0.05 | 0.8173 |
| | 26 | 0.07 | 0.7218 |
| | 28 | −0.18 | 0.3716 |
| | 35 | 0.18 | 0.3587 |
| | 42 | −0.20 | 0.3087 |
| | 49 | −0.01 | 0.9734 |
| 1 vs 3 | 14 | −0.09 | 0.6635 |
| | 21 | 0.15 | 0.4525 |
| | 22 | −0.10 | 0.4133 |
| | 23 | 0.21 | 0.2990 |
| | 26 | −0.24 | 0.2370 |
| | 28 | −0.19 | 0.3504 |
| | 35 | 0.14 | 0.4909 |
| | 42 | −0.04 | 0.8530 |
| | 49 | −0.06 | 0.7581 |
| 4 vs 5 | 14 | −0.14 | 0.3519 |
| | 21 | −0.02 | 0.8736 |
| | 22 | −0.17 | 0.2499 |
| | 23 | −0.06 | 0.7041 |
| | 26 | −0.23 | 0.1249 |
| | 28 | −0.14 | 0.3519 |
| | 35 | −0.27 | 0.0707 |
| | 42 | −0.36 | 0.0188 |
| | 49 | −0.07 | 0.6240 |

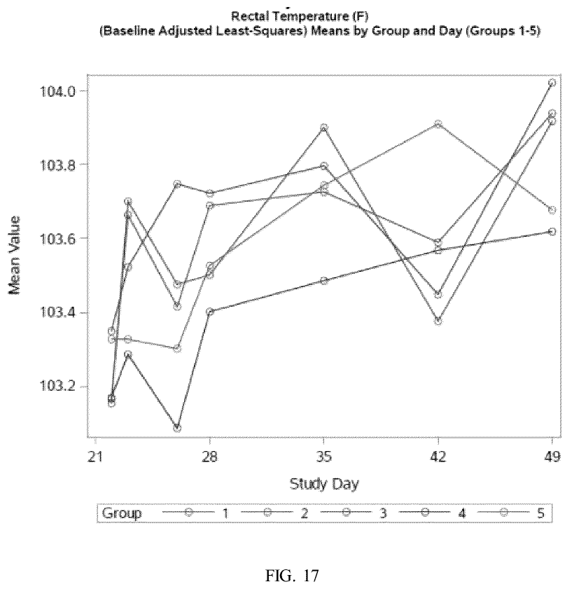

FIG. 17 is a line chart illustrating the mean rectal temperatures of animals (Baseline Adjusted Least-Squares) by Group and Day for Groups 1-5.

Table 73 shows a comparison of P-values for the various groups (1-5).

TABLE 73
Rectal Temperature (F.)
(Baseline Adjusted) Group
Comparison P-values

| Group | | | |
|---|---|---|---|
| Comparison | Day | estimate | P-value |
| 1 vs 2 | 22 | −0.01 | 0.9355 |
| | 23 | 0.04 | 0.8392 |
| | 26 | 0.06 | 0.7705 |
| | 28 | −0.19 | 0.2529 |
| | 35 | 0.17 | 0.3463 |
| | 42 | −0.21 | 0.2804 |
| | 49 | −0.02 | 0.9129 |
| 1 vs 3 | 22 | −0.20 | 0.2341 |
| | 23 | 0.18 | 0.3264 |
| | 26 | −0.27 | 0.2030 |
| | 28 | −0.22 | 0.1819 |
| | 35 | 0.10 | 0.5758 |
| | 42 | −0.07 | 0.7193 |
| | 49 | −0.10 | 0.5977 |
| 4 vs 5 | 22 | −0.16 | 0.2454 |
| | 23 | −0.04 | 0.6069 |
| | 26 | −0.22 | 0.0906 |
| | 28 | −0.13 | 0.3229 |
| | 35 | −0.26 | 0.0479 |
| | 42 | −0.34 | 0.0171 |
| | 49 | −0.06 | 0.7374 |

The following data relate to body weight (kg) data measured in animal subjects and the analysis thereof.

As is shown in Table 74, body weight values measured in kilograms are depicted by group for animals on a selection of study day.

TABLE 74
Body Weight (Kg) Data Listing

| | | Study Day | | | | | |
|---|---|---|---|---|---|---|---|
| Group | Animal | 13 | 21 | 28 | 35 | 42 | 49 |
| 1 | 7 | 2.9 | 4.8 | 8.0 | 11.8 | 16.3 | 20.1 |
| | 8 | 3.7 | 5.6 | 8.8 | 12.7 | 17.4 | 21.8 |
| | 9 | 3.6 | 5.6 | 8.6 | 12.0 | 15.4 | 18.8 |
| | 10 | 3.0 | 4.7 | 8.3 | 12.2 | 15.9 | 18.5 |
| | 11 | 3.0 | 4.4 | 8.0 | 12.1 | 16.7 | 19.2 |
| | 12 | 3.2 | 4.7 | 7.9 | 12.2 | 16.3 | 20.1 |
| | 13 | 2.2 | 3.4 | 5.2 | 9.4 | 13.0 | — |
| | 14 | 2.4 | 3.7 | 6.0 | 9.6 | 14.7 | 17.1 |
| 2 | 15 | 3.3 | 5.0 | 8.3 | 12.3 | 12.4 | 20.7 |
| | 16 | 4.7 | 7.3 | 11.9 | 17.3 | 22.9 | 27.6 |
| | 17 | 2.5 | 3.8 | 6.3 | 8.9 | 13.2 | 15.6 |
| | 18 | 3.4 | 5.2 | 8.0 | 11.5 | 15.9 | 19.7 |
| | 19 | 2.9 | 4.1 | 6.5 | 9.4 | 12.7 | 16.0 |
| | 20 | 4.8 | 7.4 | 11.7 | 16.5 | 21.6 | 26.3 |
| | 21 | 3.4 | 5.6 | 9.1 | 14.0 | 17.7 | 19.9 |
| | 22 | 2.9 | 4.2 | 6.3 | 10.3 | 14.6 | 17.4 |
| 3 | 23 | 3.6 | 5.8 | 8.7 | 11.9 | 16.7 | 20.7 |
| | 24 | 3.0 | 4.5 | 7.2 | 11.2 | 15.4 | 20.1 |
| | 25 | 3.3 | 5.4 | 8.9 | 12.8 | 17.1 | 20.6 |
| | 26 | 4.7 | 7.5 | 10.5 | 13.1 | 18.0 | 23.4 |
| | 27 | 3.9 | 5.7 | 8.7 | 12.7 | 16.9 | 20.8 |
| | 28 | 3.8 | 5.1 | 8.0 | 11.9 | 16.2 | 19.5 |
| | 29 | 3.7 | 6.0 | 10.1 | 14.0 | 19.4 | 23.2 |
| | 30 | 3.6 | 4.8 | 8.4 | 12.9 | 18.1 | 22.8 |
| 4 | 31 | 3.2 | 4.8 | 8.1 | 12.1 | 17.0 | 21.7 |
| | 32 | 3.3 | 5.1 | 8.3 | 11.4 | 16.3 | 20.5 |
| | 33 | 4.0 | 5.7 | 9.4 | 12.7 | 17.7 | 22.1 |
| | 34 | 3.1 | 5.1 | 8.5 | 11.3 | 15.9 | 19.9 |
| | 35 | 3.8 | 5.8 | 10.2 | 13.9 | 18.6 | 21.9 |
| | 36 | 4.8 | 7.2 | 12.0 | 15.0 | 20.9 | 25.5 |
| | 37 | 3.9 | 5.8 | 9.5 | 12.6 | 17.3 | 21.6 |
| | 38 | 4.2 | 6.2 | 9.8 | 14.3 | 19.8 | 25.0 |
| | 39 | 3.7 | 5.6 | 8.8 | 12.3 | 17.2 | 21.6 |
| | 40 | 3.5 | 5.0 | 8.4 | 11.8 | 17.8 | 22.6 |
| | 41 | 3.8 | 5.5 | 7.9 | 12.2 | 18.0 | 22.6 |
| | 42 | 3.9 | 6.4 | 10.9 | 15.2 | 21.9 | 25.5 |
| 5 | 43 | 3.6 | 5.4 | 8.6 | 11.3 | 16.5 | 21.1 |
| | 44 | 2.7 | 4.2 | 7.4 | 11.3 | 15.5 | 19.6 |
| | 45 | 3.0 | 4.5 | 7.8 | 12.0 | 16.6 | 20.3 |
| | 46 | 4.7 | 6.8 | 10.8 | 15.0 | 20.1 | 24.4 |
| | 47 | 4.4 | 5.7 | 8.9 | 12.2 | 17.2 | 21.2 |
| | 48 | 4.4 | 6.6 | 10.9 | 14.7 | 20.8 | 24.9 |
| | 49 | 4.2 | 6.1 | 9.8 | 13.4 | 18.9 | 23.7 |
| | 50 | 4.4 | 6.1 | 9.9 | 14.1 | 19.2 | 24.0 |
| | 51 | 2.4 | 4.0 | 6.8 | 10.1 | 14.8 | 19.0 |
| | 52 | 2.8 | 4.6 | 7.3 | 10.9 | 15.5 | 20.7 |
| | 53 | 2.6 | 4.3 | 7.7 | 11.2 | 16.1 | 20.6 |
| | 54 | 4.1 | 5.8 | 9.4 | 13.5 | 19.7 | 25.3 |
| 6 | 55 | 3.1 | 4.1 | 6.2 | 10.0 | 15.0 | 20.1 |
| | 56 | 2.8 | 4.2 | 7.7 | 11.6 | 16.0 | 22.2 |
| | 57 | 3.7 | 4.8 | 8.0 | 12.0 | 16.5 | 18.1 |
| | 58 | 4.0 | 5.4 | 8.0 | 11.7 | 15.1 | 19.1 |
| | 59 | 3.6 | 5.8 | 10.0 | 12.3 | 15.4 | 21.8 |
| | 60 | 3.3 | 4.0 | 6.2 | 10.5 | 15.1 | 19.7 |
| 7 | 1 | 4.0 | 5.7 | 9.5 | 15.0 | 18.7 | 24.8 |
| | 2 | 4.4 | 6.4 | 10.6 | 16.4 | 21.4 | 27.0 |
| 8 | 3 | 4.9 | 8.0 | 12.3 | 17.8 | 21.8 | 25.5 |
| | 4 | 3.9 | 6.2 | 9.7 | 15.4 | 19.8 | 24.5 |
| 9 | 5 | 3.0 | 3.9 | 6.3 | 10.6 | 13.5 | 16.9 |
| | 6 | 2.9 | 2.7 | 6.6 | 10.7 | 13.9 | 16.6 |

FIG. 18 shows the arithmetic mean body weight values for Groups 1-6 from fourteen to forty-nine days.

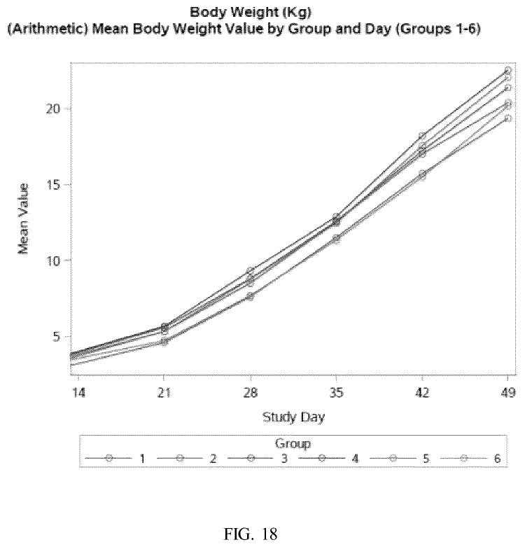

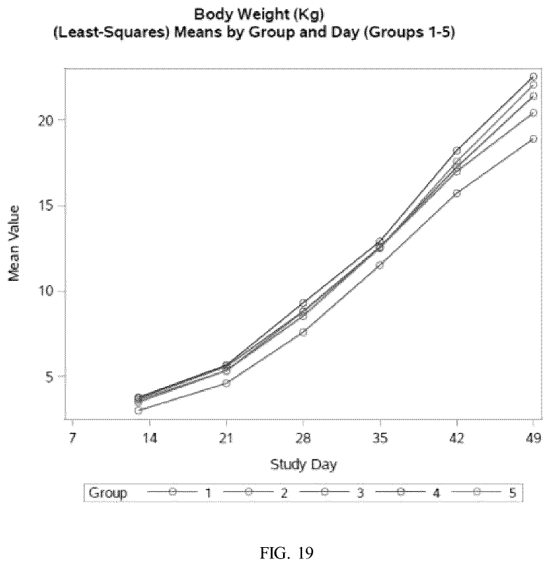

FIG. 19 is a line graph showing the body weight (Least-Squares) means by Group and Day for Groups 1-5.

Table 96 is a group comparison P-values for body weight.

TABLE 96
Body Weight (Kg)
Group Comparison P-values

| Group Comparison | Day | estimate | P-value |
|---|---|---|---|
| 1 vs 2 | 13 | −0.49 | 0.1409 |
| | 21 | −0.71 | 0.1971 |
| | 28 | −0.91 | 0.2752 |
| | 35 | −1.03 | 0.3117 |
| | 42 | −1.29 | 0.2718 |
| | 49 | −1.48 | 0.2998 |
| 1 vs 3 | 13 | −0.70 | 0.0353 |
| | 21 | −0.99 | 0.0747 |
| | 28 | −1.21 | 0.1478 |
| | 35 | −1.06 | 0.2944 |
| | 42 | −1.51 | 0.1972 |
| | 49 | −2.47 | 0.0853 |
| 4 vs 5 | 13 | 0.16 | 0.5802 |
| | 21 | 0.33 | 0.3429 |
| | 28 | 0.54 | 0.3284 |
| | 35 | 0.42 | 0.4901 |
| | 42 | 0.62 | 0.4249 |
| | 49 | 0.47 | 0.5637 |

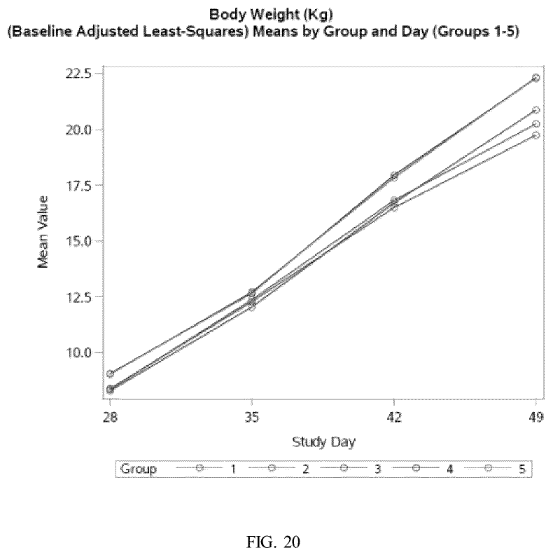

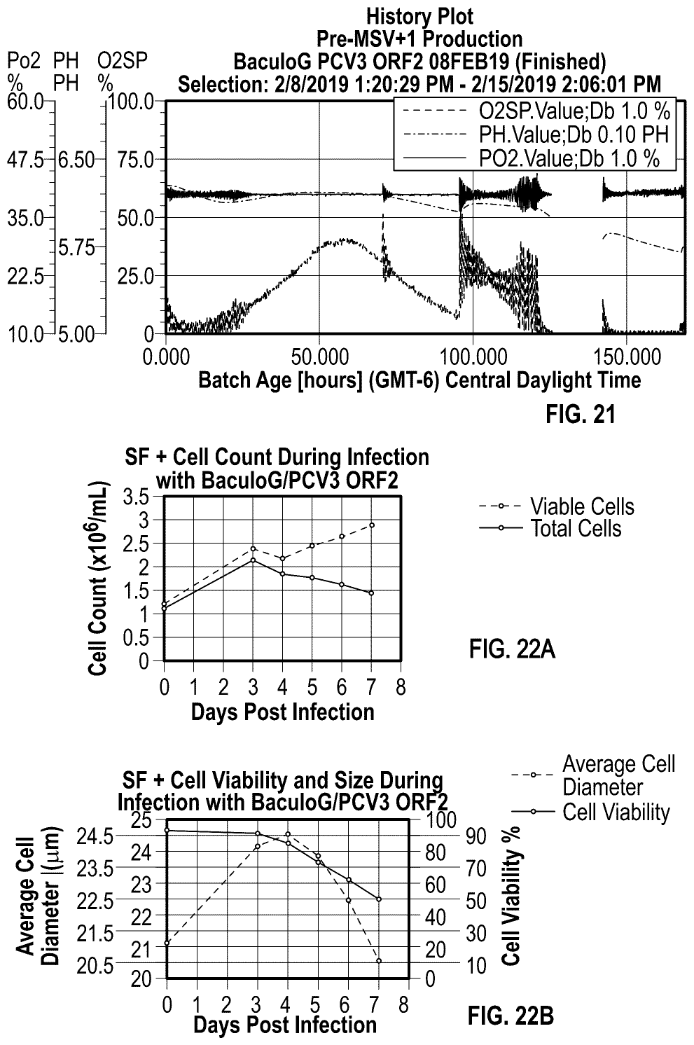

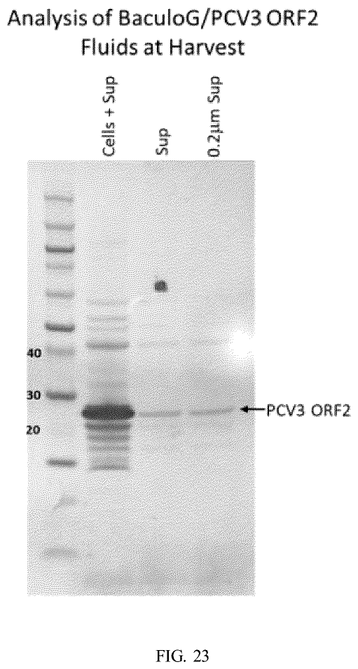

FIG. 20 is a line graph depicting data for Least-Squares Means for body weight (Baseline Adjusted) by Group and day.

Table 119 is a table of the Comparison P-values for the various groups (1-5) baseline adjusted body weights.

TABLE 119
Body Weight (Kg)
(Baseline Adjusted)
Group Comparison P-values

| Group | | | |
|---|---|---|---|
| Comparison | Day | estimate | P-value |
| 1 vs 2 | 28 | 0.04 | 0.8895 |
| | 35 | −0.07 | 0.8912 |
| | 42 | −0.34 | 0.6344 |
| | 49 | −0.49 | 0.5923 |
| 1 vs 3 | 28 | 0.10 | 0.7072 |
| | 35 | 0.25 | 0.6418 |
| | 42 | −0.20 | 0.7836 |
| | 49 | −1.11 | 0.2258 |
| 4 vs 5 | 28 | 0.05 | 0.7993 |
| | 35 | −0.07 | 0.8086 |
| | 42 | 0.13 | 0.7314 |
| | 49 | −0.02 | 0.9671 |

### Example 8

### PCV3 Bioprocess

Infection was accomplished via a baculovirus seed, BaculoG/PCV3 ORF2 Pre-MSV. The target MOI was 0.1 and the final calculated MOI was 0.1.

Infection parameters are shown in Table 120.

TABLE 120

| Parameter | Details |
|---|---|
| Vessel | 10 L Sartorius Biostat B glass-jacketed vessel |
| size/ | Ring sparger |
| configuration | 2 × 45° pitched-blade impeller with negative |
| | slope mounted approximately one inch to seven |
| | inches from the bottom of the agitator shaft |
| Working volume | 8 L |
| Media | Ex-Cell 420 Serum-Free Medium |
| Cells and | SF+ Cells @ 1 × 10⁶ cells/mL |
| target CPD | |
| Temperature | 27° C. |
| Gas/DO control | Sparge O₂ at 0.3 slpm with duty |
| | cycle controlled DO controller |
| pH control | Monitor only |
| Agitation | 100 rpm |

Bioreactor fluids were harvested aseptically into a 10 L biotainer, dispensed into 8×1 L centrifuge bottles, and centrifuged at 10,000×g for 20 min at 4° C. The clarified fluids were aseptically collected into a 10 L biotainer and filtered through 0.8/0.2 mm filter into a new 10 L biotainer and the final filtered harvest was stored at 4° C.

Ten different inactivation conditions were investigated at 5 mM binary ethyleneimine (BEI) at 37° C. for 72 hours as shown in Table 121. Samples were taken to monitor pH (Table 122) and PCV3 ORF2 solubility.

TABLE 121

| | | Control + | 150 mM | 350 mM | 550 mM | 50 mM | 100 mM | 200 mM | | |
|---|---|---|---|---|---|---|---|---|---|---|
| | Control | WFI | NaCl | NaCl | NaCl | MgCl₂ | MgCl₂ | MgCl₂ | pH 7.0 | pH 7.5 |
| Baculovirus Harvest | 220 | 220 | 220 | 220 | 220 | 220 | 220 | 220 | 220 | 220 |
| (mL) | | | | | | | | | | |
| 2M MgCl₂ (mL) | 0 | 0 | 0 | 0 | 0 | 6.3 | 12.5 | 25 | 0 | 0 |
| 5M NaCl (mL) | 0 | 0 | 7.5 | 17.5 | 27.5 | 0 | 0 | 0 | 0 | 0 |
| 1M Tris HCl, pH 7.5 | 0 | 0 | 0 | 0 | 0 | 0 | 0 | 0 | 11.3 | 25 |
| (mL) | | | | | | | | | | |
| WFI (mL) | 0 | 30 | 22.5 | 12.5 | 2.5 | 23.7 | 17.5 | 5 | 18.7 | 5 |
| Total Volume (mL) | 220 | 250 | 250 | 250 | 250 | 250 | 250 | 250 | 250 | 250 |

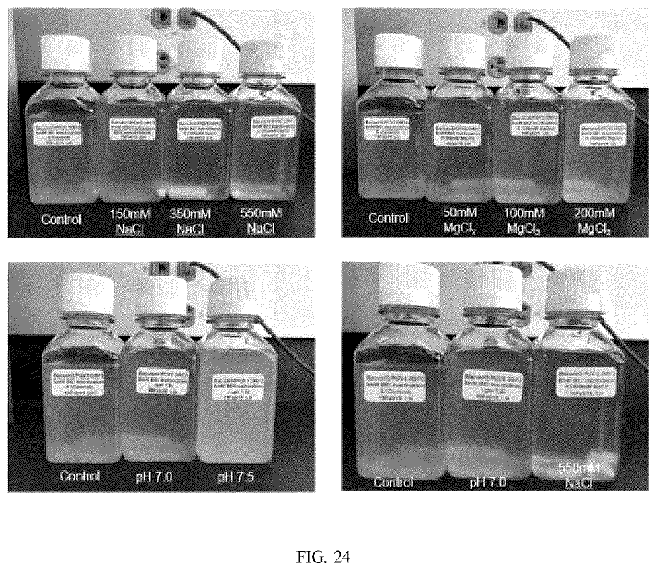

FIG. 24 shows images of inactivations at 72 hours.

TABLE 122

| Group | Treatment | Day 0 pH | Day 3 pH |
|---|---|---|---|
| A | Control | 5.89 | 6.09 |
| B | Control + WFI | 5.97 | 6.05 |
| C | 150 mM NaCl | 5.82 | 6.11 |
| D | 350 mM NaCl | 5.82 | 6.07 |
| E | 550 mM NaCl | 5.74 | 6.07 |
| F | 50 mM MgCl₂ | 5.66 | 5.91 |
| G | 100 mM MgCl₂ | 5.60 | 5.80 |
| H | 200 mM MgCl₂ | 5.46 | 5.62 |
| I | pH 7.0 | 7.00 | 6.99 |
| J | pH 7.5 | 7.42 | 7.36 |

FIG. 25 shows a western comparison of inactivation conditions for BaculoG/PCV3 ORF2 Antigen—Post Inactivation.

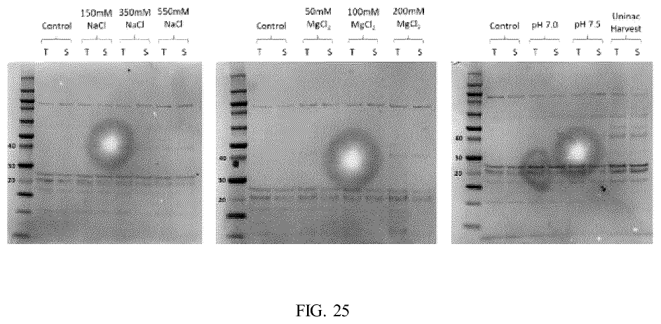

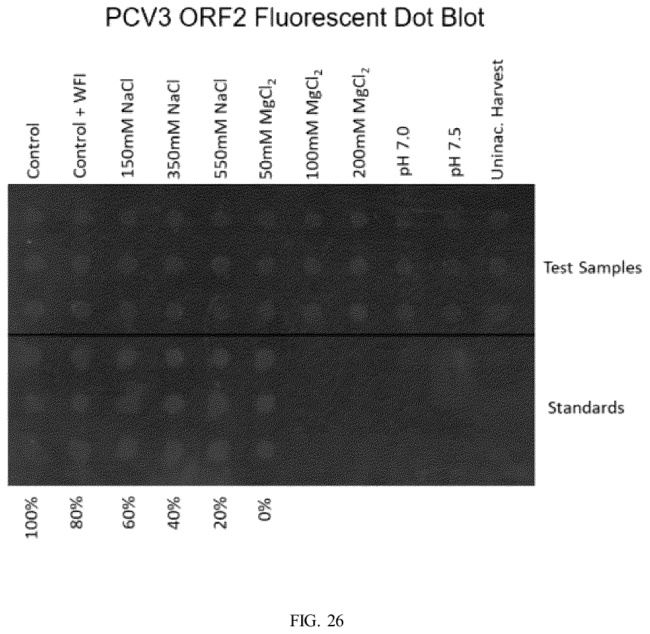

FIG. 26 shows a PCV3 ORF2 Fluorescent Dot Blot.

From this study, a low level of PCV3 ORF2 produced from the bioreactor that are similar to runs in spinners and infection kinetics suggest slight under seeding. There was minimal loss of PCV3 ORF2 from bioreactor harvest after inactivation as each of the conditions had minimal impact on soluble ORF2 although MgCl₂ at 200 mM may have had more of an effect on the observed PCV ORF2 loss. There was no loss of PCV3 ORF2 in harvest fluids stored at 4° C. for 9 days.

### Example 9

Vaccination of Caesarian-Derived Colostrum-Deprived Pigs with an Inactivated Baculovirus-Expressed PCV3 ORF2 Vaccine

This study evaluated an experimental inactivated baculovirus-expressed PCV3 ORF2 vaccine when administered to 3-week-old pigs. The randomized, blinded, vaccination-challenge study was conducted using 50 Caesarean-derived, colostrum-deprived (“CDCD”) pigs that were 22 days of age on D0. Pigs were blocked by litter and randomized to treatment group with littermates housed in the same pen. On D0, pigs received cither control product of product-matched placebo or experimental vaccine intramuscularly. Pigs were observed daily for general health, and blood was collected to evaluate seroconversion. All pigs were challenged 14 days post-vaccination with tissue homogenate (which tested positive for PCV3 by PCR) and were necropsied four weeks later. Clinical observations were conducted daily post-challenge until study off-test (D42), and blood was collected weekly to evaluate viremia (PCV3 by PCR). At necropsy, internal organs were evaluated for gross lesions, and tissues were collected for histopathologic assessment. The histology slides were stained, evaluated by RNAScope, and scored.

Throughout the study, any personnel involved in collecting data or performing laboratory assays did not know the allocation of treatment to pigs. Treatments were administered by the Study Investigator, who was not involved with any data collection.

The use of 20 animals in the control and vaccine groups, respectively, is consistent with prior vaccination-challenge licensing studies for porcine circovirus. Extra animals were included to account for natural attrition of CDCD pigs prior to challenge.

Pigs were blocked by litter and randomly assigned to treatment making the individual pig the experimental unit.

All randomizations were conducted using SAS version 9.4. Litters of six (7 litters) or eight (1 litter) pigs were utilized. For randomization to treatment group, a random variate was generated for each pig using the RANUNI function in SAS. Pig IDs were then sorted based on litter and variate values. Within each litter, the three (or four for the litter with 8 pigs) animals with the smallest variate values were assigned to T01, and the remainder assigned to T02. During the vaccination phase, pigs were housed by litter to the degree possible, with three or four pigs per brooder using brooders in three rooms. During the challenge phase, with the exception of the litter with 8 pigs, pigs were housed by litter, one litter per pen, in one of two rooms. The litter with 8 littermates was housed in two pens with 6 and 2 littermates, respectively, in a pen. For the challenge housing, litters were randomized to room and pen within room by generating a random variate for each litter, sorting by variate value, and aligning the sorted order to room-pen combinations.

Vaccination phase: Twenty-five Caesarian-derived colostrum-deprived (CDCD) pigs at approximately 3 weeks of age were vaccinated with either baculovirus expressed PCV3 ORF2 vaccine or a placebo matched control vaccine. The virus titer was determined to be 6.76×10⁶ TCID₅₀/mL. The inactivated antigen was formulated with 20% Carbopol and dispensed into final containers. The placebo used as a negative control was prepared in the same manner with inactivated antigen from a negative control baculovirus. The vaccine or placebo was administered intramuscularly to each animal. Cord blood was collected from all pigs at delivery (C-section; Day −22). Serum was separated and tested for PCV3 DNA and PCV2 DNA by PCR. All samples were negative for both PCV3 and PCV2. On D-2, all pigs were bled for serum collection and then vaccinated with PCV2 Ingelvac CircoFLEX®. All serum samples were negative by PCR for both PCV3 and PCV2 and seronegative for M. hyopneumoniae and Porcine Reproductive and Respiratory Syndrome Virus.

Challenge phase: All animals were challenged with PCV3 positive tissue homogenate (1 mL intranasally and 1 mL intramuscularly) at 14 days post vaccination. Keyhole limpet hemocyanin (KLH) emulsified in incomplete Freund's adjuvant (ICFA) containing 1 mg KLH/mL was administered intramuscularly two days before and two days after challenge (Table 123). The tissue homogenate used for challenge was screened for extraneous agents by qPCR and deep sequencing. Animals were euthanized at day 42. At necropsy, a number of tissues were collected. These included brain, heart, kidney, lung, spleen, large intestine, tonsil, tracheobronchial lymph node (TBLN), mesenteric lymph node (MLN), and external iliac lymph node (ILN).

TABLE 123
Study Design

| Group | N | Vaccination | KLH/ICFA | Challenge | KLH/ICFA | Necropsy |
|---|---|---|---|---|---|---|
| Placebo | 25 | D0 | D12 | D14 | D16 | D42 |
| Vaccine | 25 | 2 mL IM | 2 mL IM | 1 mL IN/1 mL | 2 mL IM | |
| | | (right neck) | | IM | | |

Table 124 describes the tissue homogenate used to challenge the animals to PCV3.

TABLE 124

| | |
|---|---|
| Description | PCV3 pluck tissue homogenate; Lot # 3743-105, Ct = 9.5 |
| Formulation | Frozen tissue ground using sterile mortar and pestle, suspended in MEM, and |
| | spun at 1000 g for 15 min. Supernatant filtered through 0.2 μm filter and |
| | stored at −70° C. ± 10° C. until use. One day prior to challenge, material was |
| | thawed at 37° C., bottled into sterile vaccine-type bottles and capped. |
| Dosage and | 1 mL intranasally by attaching a nasal tip atomizer to a 5 cc luer lock syringe |
| challenge | and applying the full 1-mL dose into one nostril. 1 mL intramuscularly in the |
| procedure | left neck muscle neck midway between the base of the ear and point of the |
| | shoulder using appropriate-sized sterile syringes and sterile needles. |
| Testing | Routine culture of the material was conducted on blood agar plates at 37° C. |
| | anaerobically and aerobically for 48 hours; no growth was observed and |
| | the test was considered satisfactory. The material was tested by PCR for the |
| | presence of mycoplasma and PCV2; no contamination was identified. The |
| | PCV3 qPCR result was 9.1 log₁₀ genomic copies/mL (Cq = 14.82). Deep |
| | sequencing was completed on the samples (MiSeq_127 9 Oct. 2018) using both |
| | DNA and RNA processing; sequencing resulted in recovery of the full PCV3 |
| | genome (99% nt to PCV3 GB MG564174.1). |

Table 125 provides information regarding the pigs used in the study.

TABLE 125

| Specifications | Requirements |
|---|---|
| Species & breed | Porcine, commercial mixed breed |
| Age & sex | Pigs were 22 days of age at D0 (born by |
| | caesarian section), both females and males |
| Weight range | Typical weight for CDCD pigs of this age |
| Source & | CDCD pigs were derived and raised at Struve |
| ownership | Labs International; 1603 Enterprise St., |
| | Manning, Iowa 51455 |
| Number | 50 (seven litters of 6 pigs, one litter of 8 pigs) |
| Identification | Ear tag (uniquely numbered) |
| Conditioning | A venous blood sample was collected from all |
| | pigs at delivery (cord blood at C-section; D- |
| | 22). Serum was shipped on ice or frozen |
| | immediately and tested for PCV3 DNA and |
| | PCV2 DNA by PCR at ISU-VDL to establish |
| | sero-status for all available pigs. All samples |
| | were negative for both PCV3 and PCV2. |
| | On D-2, all pigs were bled for serum |
| | collection and then vaccinated with PCV2 |
| | Ingelvac CircoFLEX ®. Serum was shipped |
| | on ice or frozen to BI AH USA-Ames |
| | immediately and tested for PCV3 DNA and |
| | PCV2 DNA by PCR at ISU-VDL. All |
| | samples were negative by PCR for both |
| | PCV3 and PCV2. Retention samples of D-2 |
| | sera (except pig #18 and #45 because of a |
| | lack of sera) were submitted to ISU-VDL |
| | to confirm seronegative status for M. |
| | hyopneumoniae (S/P ratio <0.3), and PRRSV |
| | (S/P ratio <0.4). All samples were negative. |
| Veterinary care | Pigs received a medicated feed ration. On D 8, |
| and treatment | pigs received a label dose of Excede ® prior to |
| | shipment to VRI. Because of suspected |
| | bacterial sepsis, all remaining pigs received |
| | Baytril (lot #AHO2X32, exp November 2021) |
| | on D 30 in the left neck via label directions. |

All 50 pigs met requirements outlined above, and the Study Investigator conducted a Health Examination on D-2 to ensure only healthy animals were included in the study.

After the start of the study, pigs were to be removed only in the case of injury, illness, or death that would interfere with the outcome of the study. Two pigs were removed during the vaccination phase, and five pigs were removed during the challenge phase.

Pig #5 (vaccinated group) was observed with lack of appetite and depressed on D6; the pig was euthanized and removed from the study on D6. Necropsy revealed icteric skin, subcutis fascia, and fibrin on liver and spleen with a mottled liver surface, and a collapsed left apical lung lobe. The carcass was disposed by composting.

Pig #4 (vaccinated group) was euthanized and removed from the study prior to challenge on D14 because of poor body condition and lameness in both rear legs. No gross lesions were observed at necropsy. The carcass was disposed by incineration.

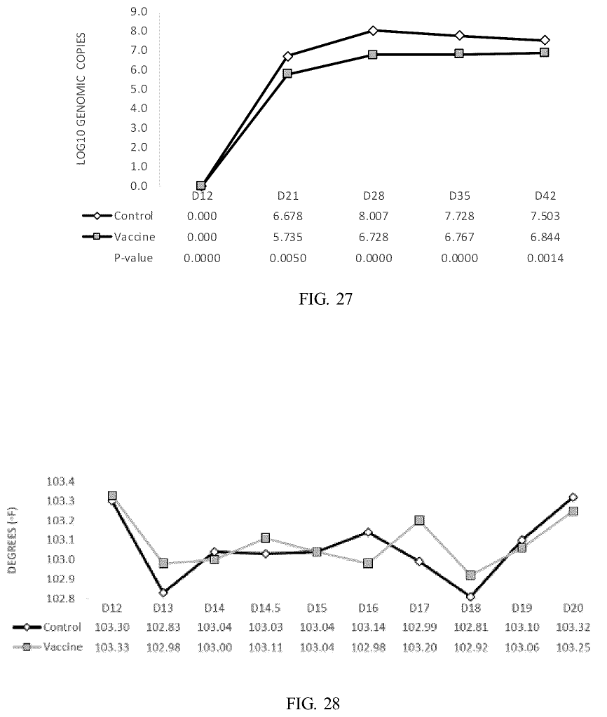

Viremia: Viremia was defined as PCV3 positive results by PCR (cycle threshold (Ct) value <37, genomic equivalence 4.697 logs for this study). Post-challenge viremia in vaccinates and control animals was evaluated at by qPCR. All control pigs were viremic at each sampling point during the challenge phase (Table 126). Three vaccinated pigs had positive results on D7 with Ct-values of 35.6 to 36.7 (with≥37 being the cut-off for negative), which most likely indicated a false positive result considering all vaccinated pigs were negative at D12, pre-challenge. Following challenge, two vaccinated pigs did not become viremic. While up to 91% of the vaccinated pigs did become viremic, the load of virus (genomic copies) in the blood was significantly reduced by approximately a log in the vaccinates at each post-challenge time point (P≤0.0050) compared to the controls (FIG. 27).

TABLE 126
Frequency and percent of PCV3 viremic pigs by treatment and day

| | Study Day (vaccination on D0, PCV3 challenge on D14) | | | | | | | | | | | | | |
|---|---|---|---|---|---|---|---|---|---|---|---|---|---|---|
| Group | D-2 | | D7 | | D12 | | D21 | | D28 | | D35 | | D42 | |
| Control | 0/25 | (0%) | 0/25 | (0%) | 0/25 | (0%) | 25/25 | (100%) | 25/25 | (100%) | 25/25 | (100%) | 23/23 | (100%) |
| Vaccine | 0/25 | (0%) | 3/13 | (13%) | 0/24 | (0%) | 8/23 | (35%) | 20/22 | (91%) | 18/20 | (90%) | 18/20 | (90%) |

General health observations: All pigs were observed daily for general health from D-2 until D14 with an additional observation between 2 and 4 hours post-vaccination. No clinical signs were seen after vaccination until D6 when pig #5 (vaccinated group) was observed with loss of appetite and depression prior to being removed from the study for humane reasons. On D8, pigs #12 (vaccinated group), #15 (placebo group), #36 (vaccinated group), and #38 (placebo group) were observed with hernias. On D9, pig #4 (vaccinated group) was found stuck in between the feeder and wall prior to being transported to VRI; at VRI the pig was found to be lame with a swollen right rear leg which progressed to bilateral rear leg lameness on D11 before being removed from the study prior to challenge on D14.

Post-challenge mortality: Pigs that died or were euthanized post-challenge prior to off-test on D42, were necropsied. Two control pigs (placebo group) and three vaccinated pigs (vaccinated group) died or were euthanized during the challenge phase. Pig #7 (vaccinated group) was found dead on D26. Necropsy observations were congested meningeal vessels and enlarged ILN. Pig #2 (vaccinated group) was found dead on D30. Necropsy observations included chronic-active fibrosing and fibrinous pericarditis and cranial ventral pneumonia. Pig #19 (vaccinated group) was found dead on D31. The pig was observed as small with no gross necropsy lesions suggesting failure to thrive. Pig #49 (placebo group) was found dead on D35 with no previous clinical signs and with gross lesions of pulmonary congestion of the kidney with scant amounts of white exudate. Pig #15 (placebo group) was euthanized for humane reasons on D40. The pig was found comatose and paddling. Necropsy revealed moderate hydrocephalus and diffuse congestion of meningeal vessels. Previously the pig was ataxic for 7 days and depressed for the four preceding days. Additionally, the pig had severe respiratory signs (thumping) on D35 and reduced body condition for the 10 days prior to euthanasia.

Beginning 2 to 4 hours post-challenge and then daily during the challenge phase, all pigs were observed once daily for PCV3-associated clinical signs as described in Table 127.

TABLE 127

| | | Body | | Respiratory | |
|---|---|---|---|---|---|
| Score | Neurological Signs | Condition | Diarrhea | Signs | Dermatitis |
| 0 | Normal | Normal | Normal | Normal | Normal |
| None | | | | | |
| 1 | Depressed = depressed | depressed | slightly | mild | Red-purple |
| Mild | to lethargic, requires | appetite but | loose stool | increase in | blotches on the |
| | physical stimulation to | still eating, | observed | respiratory | skin most |
| | provoke locomotion | slightly thin | from pig | rate | obvious on the |
| | | compared | | | hind legs |
| | | to pen mates | | | |
| 2 | Ataxic = unable to | not eating, | runny, loose | notable | Slightly raised |
| Moderate | coordinate muscle | ribs and | stool | increase in | red-purple |
| | activity, spastic | backbone | observed; | respiratory | blotches on the |
| | movements involving | obviously | obvious | rate | skin, on the |
| | head, limbs, and/or trunk | pronounced | staining of | | hind legs, |
| | | | the perianal | | perineum, or |
| | | | region | | abdomen |
| 3 | Tremors = involuntary | emaciated | very watery | thumping | Red-purple |
| Severe | repetitive muscle | | stool | | blotches |
| | movements | | observed | | covering most |
| | | | | | of the body |
| 4 | Recumbent = laying | | | | |
| Severe | down, unable to raise | | | | |
| | when provoked with | | | | |
| | physical stimulus | | | | |
| 5 | Seizures = bilateral tonic | | | | |
| Severe | or clonic contraction of | | | | |
| | muscles resulting in | | | | |
| | partial or complete | | | | |
| | unconsciousness | | | | |

Clinical signs occurred between D21 and D40 with the majority being mild depression (neurologic) and mild increase in respiratory rate (Table 128). Diarrhea and dermatitis were not observed during the challenge phase.

TABLE 128

| | Control | | | | | | | | | Vaccine | | | | | |
|---|---|---|---|---|---|---|---|---|---|---|---|---|---|---|---|
| | | Neurological | | | | | | | | | Neurological | | | Respi- | |
| | | | ata- | | Respiratory | | | BC | Any | | | ata- | | ratory | Any |
| Day | n | dep | xic | recum | mild | mod | thump | mild | Sign | n | dep | xic | recum | mild | Sign |
| D21 | 25 | • | • | • | • | • | • | • | • | 23 | 4% | • | • | 4% | 9% |
| D22 | 25 | • | • | • | • | • | • | • | • | 23 | 4% | • | • | 4% | 9% |
| D23 | 25 | • | • | • | • | • | • | • | • | 23 | 4% | • | • | 4% | 9% |
| D24 | 25 | • | • | • | 4% | • | • | 4% | 8% | 23 | 4% | • | • | 9% | 9% |
| D25 | 25 | • | • | • | 4% | • | • | 4% | 8% | 23 | 9% | • | • | 13% | 17% |
| D26 | 25 | • | • | • | 4% | • | • | 4% | 8% | 22 | 5% | • | • | 9% | 9% |
| D27 | 25 | • | • | • | 20% | • | • | 4% | 24% | 22 | 5% | • | • | 23% | 23% |
| D28 | 25 | 4% | • | • | 16% | • | • | • | 20% | 22 | 5% | • | • | 23% | 23% |
| D29 | 25 | 16% | 4% | • | 16% | • | • | • | 28% | 22 | 5% | • | 5% | 23% | 27% |
| D30 | 25 | 8% | 4% | • | 12% | • | • | 4% | 24% | 22 | 9% | • | 5% | 14% | 27% |
| D31 | 25 | 12% | • | • | 8% | • | • | 4% | 20% | 21 | 5% | • | 5% | 19% | 29% |
| D32 | 25 | 4% | • | • | • | • | • | 4% | 4% | 20 | 5% | • | • | 10% | 15% |
| D33 | 25 | • | 4% | • | • | • | • | 4% | 4% | 20 | • | 5% | • | 10% | 15% |
| D34 | 25 | • | 4% | • | • | • | • | 4% | 4% | 20 | • | 5% | • | • | 5% |
| D35 | 24 | • | | • | 4% | 8% | 4% | | 17% | 20 | • | • | • | • | • |
| D36 | 24 | • | 4% | • | 17% | 8% | • | 8% | 25% | 20 | 5% | • | • | 5% | 10% |
| D37 | 24 | • | 4% | • | 17% | 4% | • | 4% | 21% | 20 | 5% | • | • | 5% | 10% |
| D38 | 24 | • | 4% | • | 21% | • | • | 4% | 25% | 20 | • | • | • | 5% | 5% |
| D39 | 24 | • | 4% | • | 8% | • | • | 4% | 13% | 20 | • | • | • | 5% | 5% |
| D40 | 24 | • | • | 4% | • | • | • | 4% | 4% | 20 | • | • | • | 5% | 5% |
| D41 | 23 | • | • | • | • | • | • | • | • | 20 | • | • | • | • | • |
| D42 | 23 | • | • | • | • | • | • | • | • | 20 | • | • | • | • | • |

| | | | | | | | | | | | | | | |
|---|---|---|---|---|---|---|---|---|---|---|---|---|---|---|
| Pigs in | 5 | 2 | 1 | 14 | 4 | 1 | 3 | 14/25 | • | 4 | 1 | 2 | 11 | 11/23 |
| Each | | | | | | | | (56%) | | | | | | (48%) |
| Category | | | | | | | | | | | | | | |

Diarrhea and dermatitis were not seen during the study.
Only pigs with an observation are shown:
BC = Body Condition,
dep = depressed,
recum = recumbent,
mod = moderate,
thump = thumping

Body weights: All pigs were weighed prior to vaccination, prior to challenge, one week following challenge, and prior to necropsy. Least squares means body weight for the vaccine group was numerically (not significantly) heavier at each time point (Table 129).

TABLE 129

| Group | D-2 | D 12 | D 21 | D 42 |
|---|---|---|---|---|
| Control | 4.04 kg | 7.13 kg | 13.20 kg | 39.11 kg |
| Vaccine | 4.06 kg | 7.40 kg | 13.78 kg | 40.70 kg |

Body temperatures: Body temperatures were measured by self-calibrating rectal thermometer at and by intradermal microchips. Temperatures were measured three times prior to challenge to establish a baseline, then 2 to 4 hours post-challenge, and once daily until D20. Mean temperatures for treatment groups were within 1° F. on each of the days of collection (FIG. 28).

Gross lesion evaluation: Post-challenge, all pigs were necropsied at time of death or at scheduled off-test (D42). The Study Investigator performed a post-mortem assessment of all major organ systems. Specific pathological descriptions were included for the lymph nodes (tracheobronchial, external inguinal, mesenteric), kidney, heart, and lungs (Table 130).

TABLE 130

| Score | Lymph nodes | Lungs | Dermatitis | Kidney | Heart |
|---|---|---|---|---|---|
| 0 | normal | normal | normal | normal | normal |
| None | | | | | |
| 1 | Enlarged, but | Interstitial | Red-purple | Enlarged | Enlarged |
| Mild | less than 2 | pneumonia | blotches on the | | |
| | times greater | | skin most obvious | | |
| | than normal | | on the hind legs | | |
| 2 | Enlarged 2-5× | Multifocal areas | Slightly raised red- | Multifocal white or | Multifocal pale |
| Moderate | normal | of consolidation | purple blotches on the | red pinpoint lesions | (necrotic) or red |
| | | | skin, on the hind legs | with or without | (hemorrhagic) areas |
| | | | and perineum or the | enlargement | present |
| | | | abdomen | | |

| | | | |
|---|---|---|---|
| 3 | Enlarged, greater | Diffuse | Red-purple blotches |
| Severe | than 5× normal | consolidation | covering most of the |
| | | with interstitial | body |
| | | pneumonia | |

Very few lesions were observed upon necropsy. No lesions were seen in the heart, kidney, or skin (dermatitis). Multifocal congestion of the lung was seen in three vaccinated pigs, one of which was a mortality. Comments confirmed the lesions as minimal (1%). The balance of lesions were enlarged lymph nodes, 10/25 control pigs and 14/23 vaccinated pigs.

Tissue collection & histologic scoring: At necropsy, the Study Investigator collected brain (cerebellum), heart (affected area, otherwise cross-section of the right and left ventricles), kidney (cross-section), lung (affected area, otherwise accessory lobe), spleen (cross-section), large intestine, small intestine, tonsil, tracheobronchial lymph node (TBLN), mesenteric lymph node (MLN), and inguinal lymph node (ILN). All tissues from a pig were saved in containers filled with a sufficient amount of 10% buffered formalin solution. After 24 hours in 10% buffered formalin solution, tissues were transferred to 70% ethanol and submitted for histologic slide preparation at ISU VDL. Tissue samples were processed for routine hematoxylin and eosin (H&E) staining. Each H&E slide was scored as lesions present or not. If abnormalities were noted, a brief description of the morphological diagnosis was provided along with a severity score according to Table 131.

TABLE 131

| | Severity Score | | |
|---|---|---|---|
| Tissue | None (0) | Mild (1) | Moderate (2) |
| Lymph | Normal-No | Lymphadenitis, | Meningoencephalitis, |
| Nodes | significant | granulomatous, diffuse, | lymphoplasmacytic, multifocal, |
| | histological | chronic with ≤5 multi- | subacute, mild perivasculitis with >6 |
| | lesions | nucleated giant cell and | lymphoplasmacytic foci and/or |
| | | intralesional lipid vacuoles | perivascular aggregates |
| Cerebrum | Normal-No | Meningoencephalitis, | Lymphadenitis, granulomatous, |
| & | significant | lymphoplasmacytic, | diffuse, chronic, severe with >6 |
| Cerebellum | histological | multifocal, subacute, minimal | multinucleated giant cells |
| | lesions | perivasculitis with ≤5 | |
| | | lymphoplasmacytic foci | |
| | | and/or perivascular | |
| | | aggregates | |
| Tonsil | Normal-No | Tonsillitis, granulomatous, | Lymphadenitis, granulomatous, |
| | significant | diffuse, chronic, moderate | diffuse, chronic, severe with >6 |
| | histological | with ≤5 multinucleated | multinucleated giant cells |
| | lesions | giant cells | |
| Heart | Normal-No | Myocarditis, | Myocarditis, lymphoplasmacytic, |
| | significant | lymphoplasmacytic, | multifocal, subacute, mild |
| | histological | multifocal, subacute, minimal | perivasculitis with >6 |
| | lesions | perivasculitis with ≤5 | lymphoplasmacytic foci and/or |
| | | lymphoplasmacytic foci | perivascular aggregates |
| | | and/or perivascular | |
| | | aggregates | |
| Dermis | Normal-No | Interstitial pneumonia, | Interstitial pneumonia, lympho- |
| | significant | lymphoplasmacytic, | plasmacytic, multifocal, subacute, |
| | histological | multifocal, subacute, minimal | mild perivasculitis with >6 |
| | lesions | perivasculitis with ≤5 | lympho-plasmacytic foci and/or |
| | | lymphoplasmacytic foci | perivascular aggregates |
| | | and/or perivascular | |
| | | aggregates | |
| Liver | Normal-No | Hepatitis, | Hepatitis, lymphoplasmacytic, |
| | significant | lymphoplasmacytic, | multifocal, subacute, mild |
| | histological | multifocal, subacute, minimal | perivasculitis with >6 |
| | lesions | perivasculitis with ≤5 | lymphoplasmacytic foci and/or |
| | | lympho-plasmacytic foci | perivascular aggregates |
| | | and/or perivascular | |
| | | aggregates | |
| Spleen | Normal-No | Splenitis, lymphoplasmacytic, | Splenitis, lymphoplasmacytic, |
| | significant | multifocal, subacute, minimal | multifocal, subacute, mild |
| | histological | perivasculitis with ≤5 | perivasculitis with >6 |
| | lesions | lymphoplasmacytic foci | lymphoplasmacytic foci and/or |
| | | and/or perivascular | perivascular aggregates |
| | | aggregates | |
| Kidney | Normal-No | Interstitial nephritis, | Interstitial nephritis, |
| | significant | lymphoplasmacytic, | lymphoplasmacytic, multifocal, |
| | histological | multifocal, subacute, minimal | subacute, mild perivasculitis with >6 |
| | lesions | perivasculitis with ≤5 | lymphoplasmacytic foci and/or |
| | | lymphoplasmacytic foci | perivascular aggregates |
| | | and/or perivascular | |
| | | aggregates | |
| Small | Normal-No | Intestinal serositis, | Intestinal serositis, |
| Intestine | significant | lymphoplasmacytic, | lymphoplasmacytic, multifocal, |
| | histological | multifocal, subacute, minimal | subacute, mild perivasculitis with >6 |
| | lesions | perivasculitis with ≤5 | lymphoplasmacytic foci and/or |
| | | lymphoplasmacytic foci | perivascular aggregates |
| | | and/or perivascular | |
| | | aggregates | |
| Large | Normal-No | Intestinal serositis, | Intestinal serositis, |
| Intestine | significant | lymphoplasmacytic, | lymphoplasmacytic, multifocal, |
| | histological | multifocal, subacute, minimal | subacute, mild perivasculitis with >6 |
| | lesions | perivasculitis with ≤5 | lymphoplasmacytic foci and/or |
| | | lymphoplasmacytic foci | perivascular aggregates |
| | | and/or perivascular | |
| | | aggregates | |

No histologic lesions were observed in the tonsil, TBLN, MLN, or spleen of any pig. Most all pigs had at least mild histologic lesions of the ILN (Table 132).

Overall, histologic lesions of the brain (Table 133), kidney (Table 134), heart (Table 135), and lungs (Table 136) were generally mild with only 2/25 control pigs having histologic lesions of the intestines, one small intestine (Table 137) and one large intestine (Table 138). Two pigs with histologic lesions of the brain were found dead during the study with gross lesions of meningitis (#15 [placebo group] purulent and lymphocytic meningoencephalitis and #7 [vaccinated group] bacterial chronic active meningitis). Four pigs in the placebo group and two pigs in the vaccinated group had lesions in two tissues. Overall, 92% of controls had histologic lesions, and 87% of vaccinated pigs had histologic lesions (Table 139).

TABLE 132
Frequency of inguinal lymph node histologic lesion severity

| Group | normal | mild | moderate | severe |
|---|---|---|---|---|
| Control | 4/25 (16%) | 12/25 (48%) | 8/25 (32%) | 1/25 (4%) |
| Vaccine | 5/23 (22%) | 13/23 (57%) | 4/23 (17%) | 1/23 (4%) |

TABLE 133
Frequency of brain histologic lesion severity

| Group | normal | mild |
|---|---|---|
| Control | 24/25 (96%) | 1/25 (4%) |
| Vaccine | 22/23 (96%) | 1/23 (4%) |

TABLE 134
Frequency of kidney histologic lesion severity

| Group | normal | mild | moderate |
|---|---|---|---|
| Control | 22/25 (88%) | 3/25 (12%) | — |
| Vaccine | 19/23 (83%) | 3/23 (13%) | 1/23 (4%) |

TABLE 135
Frequency of heart histologic lesion severity

| Group | normal | mild |
|---|---|---|
| Control | 20/25 (80%) | 5/25 (20%) |
| Vaccine | 20/23 (87%) | 3/23 (13%) |

TABLE 136
Frequency of lung histologic lesion severity

| Group | normal | mild |
|---|---|---|
| Control | 24/25 (96%) | 1/25 (4%) |
| Vaccine | 21/23 (91%) | 2/23 (9%) |

TABLE 137
Frequency of large intestine histologic lesion severity

| Group | normal | mild |
|---|---|---|
| Control | 24/25 (96%) | 1/25 (4%) |
| Vaccine | 23/23 (100%) | 0/23 (0%) |

TABLE 138
Frequency of small intestine histologic lesion severity

| Group | normal | mild |
|---|---|---|
| Control | 24/25 (96%) | 1/25 (4%) |
| Vaccine | 21/21 (100%) | 0/21 (0%) |

TABLE 139
Frequency of histologic lesions by group

| Group | If Ever |
|---|---|
| Control | 23/25 (92%) |
| Vaccine | 20/23 (87%) |

Virus replication in tissues of vaccinates and placebo animals post challenge was evaluated using RNAScope. RNAscope allows specific tagging and visualization of viral mRNA. RNAscope detects replicating virus in a tissue as opposed to immunohistochemistry or PCR, which identifies genetic material of a virus whether regardless of whether the virus is live or dead. Tissues were fixed and permeabilized to allow for target probe access sites of viral replication within the cells. A pair of PCV3 RNA specific oligonucleotide probes were then hybridized so as to sit within close proximity of each other on the PCV3 target RNA. The detection of mRNA means the PCV3 virus is replicating and not simply detecting PCV3 genetic material. This was followed by the hybridization of a signal amplification molecule (SAM) that recognizes the pair of specific oligonucleotide probes. In non-specific reactions, the two probes would not sit next to each other preventing their hybridization with the SAM. The SAMs themselves are conjugated to an enzyme. As in in situ hybridization assays, the signals are detected using a chromogenic substrate followed by bright-field microscopic examination of slides. Slides for PCV3 RNAscope assay were stained, read, and scored according to Table 140.

TABLE 140
RNAScope scoring scale

| Score | Description |
|---|---|
| 0 = normal | zero cells with PCV3 staining |
| 1 = mild | <10% of cells with PCV3 staining |
| 2 = moderate | 10-50% of cells with PCV3 staining |
| 3 = severe | >50% of cells with PCV3 staining |

No evidence of PCV3 replication was observed in any sections of cerebrum/cerebellum of any pig. Nearly all of the control pigs had at least mild PCV3 RNAScope staining in the kidney (Table 141), heart (Table 142), large intestine (Table 143), and small intestine (Table 144) while only one vaccinated pig had mild staining of each of the four tissues, and three other pigs had mild staining of the kidney.

All control pigs had mild to moderate staining of the spleen (Table 145) and mild to severe staining of the ILN (Table 146) and lung (Table 147). In contrast, six vaccinated pigs had no RNAscope staining in any tissue (including that of three pigs that died in the challenge phase). Looking at maximum RNAscope staining by pig, 48% of controls had a score of severe and the other 52% were moderate, compared to only 9% of vaccinated pigs having a severe score and 17% with a moderate score. All control pigs had at least one tissue with replicating PCV3 virus while 71% of vaccinated pigs had at least one tissue with replicating PCV3 virus (Table 148). A significant result of the study is the difference between control and vaccine when evaluating tissues using RNAScope (Table 149). RNAscope detects replicating virus in a tissue as opposed to immunohistochemistry or PCR, which identifies genetic material of a virus whether it is live or dead. Strikingly, all control pigs had mild to moderate staining of the spleen and mild to severe staining of the ILN and lung. In contrast, six vaccinated pigs had no RNAscope staining in any tissue. All tissues from vaccinated animals demonstrated significant prevention of infection.

TABLE 141
Frequency of kidney RNAScope Scores

| Group | normal | mild |
|---|---|---|
| Control | 2/25 (8%) | 23/25 (92%) |
| Vaccine | 19/23 (83%) | 4/23 (17%) |

TABLE 142
Frequency of heart RNAScope Scores

| Group | normal | mild | moderate |
|---|---|---|---|
| Control | 4/25 (16%) | 20/25 (80%) | 1/25 (4%) |
| Vaccine | 22/23 (96%) | 1/23 (4%) | . |

TABLE 143
Frequency of large intestinal RNAScope Scores

| Group | normal | mild |
|---|---|---|
| Control | 4/25 (16%) | 21/25 (84%) |
| Vaccine | 22/23 (96%) | 1/23 (4%) |

TABLE 144
Frequency of small intestinal RNAScope Scores

| Group | normal | mild |
|---|---|---|
| Central | 9/25 (36%) | 16/25 (64%) |
| Vaccine | 20/21 (95%) | 1/21 (5%) |

TABLE 145
Frequency of spleen RNAScope Scores

| Group | normal | mild | moderate | severe |
|---|---|---|---|---|
| Control | . | 13/25 (52%) | 12/25 (48%) | . |
| Vaccine | 9/23 (39%) | 13/23 (57%) | . | 1/23 (4%) |

TABLE 146
Frequency of inguinal lymph node RNAScope Scores

| Group | normal | mild | moderate | severe |
|---|---|---|---|---|
| Control | . | 2/24 (8%) | 16/24 (67%) | 6/24 (25%) |
| Vaccine | 9/23 (39%) | 8/23 (35%) | 6/23 (26%) | . |

TABLE 147
Frequency of lung RNAScope Scores

| Group | normal | mild | moderate | severe |
|---|---|---|---|---|
| Contral | . | 4/25 (16%) | 11/25 (44%) | 10/25 (40%) |
| Vaccine | 13/23 (57%) | 5/23 (22%) | 3/23 (13%) | 2/23 (9%) |

TABLE 148
Frequency of RNAScope Scores

| Group | If Ever |
|---|---|
| Control | 25/25 (100%) |
| Vaccine | 17/23 (71%) |

TABLE 149
Statistical comparisons of RNAScope tissue results

| | Mitigated Fraction | Proportion | | Prevented Fraction | Fisher’s |
|---|---|---|---|---|---|
| | (lower bound, | Positive | | (upper & lower | Exact Test |
| Tissue | upper bound) | Control | Vaccine | confidence interval) | P-Value |
| ILN | 0.771 (0.667, 0.879 | 1.000 | 0.609 | 0.397 (0.156, 0.569) | 0.0006 |
| Spleen | 0.616 (0.417, 0.803) | 1.000 | 0.609 | 0.395 (0.156, 0.566) | 0.0005 |
| Lung | 0.712 (0.556, 0.857) | 1.000 | 0.435 | 0.571 (0.310, 0.734) | 0.0000 |
| Kidney | . | 0.920 | 0.174 | 0.802 (0.524, 0.918) | 0.0000 |
| Heart | . | 0.840 | 0.043 | 0.941 (0.632, 0.990 | 0.0000 |
| Large | . | 0.840 | 0.043 | 0.940 (0.638, 0.990) | 0.0000 |
| Intestine | | | | | |
| Small | . | 0.640 | 0.048 | 0.889 (0.449, 0.978) | 0.0000 |
| Intestine | | | | | |
| All | 0.781 (0.623, 0.943) | 1.000 | 0.739 | 0.269 (0.059, 0.432) | 0.0082 |
| Tissues | | | | | |

The study was valid based on the control pigs remaining seronegative for PCV3 through the vaccination period. Clinical disease was demonstrated with clinical signs of depression (neurologic) and increase in respiratory rate between 7 and 26 days post-challenge, mortality, weight gain, viremia, gross lesions, microscopic lesions, and RNAScope results.

Statistical analysis of data was conducted using SAS version 9.4 (SAS, Cary, North Carolina/USA, SAS Institute, Inc.). Data listings and summary statistics by treatment group were generated for all variables, as appropriate.

For necropsy, histopathologic, clinical observations, pyrexia and PCV3 RNA Scope assessments, methods for data analysis varied depending on the distribution of the data for the variable under assessment. In general, data were analyzed using methods described below for the Prevented Fraction (PF) and Fisher's Exact Test, and/or Mitigated Fraction (MF). For some variables, nearly all/all responses were in one category and thus no analysis was conducted. Mortality was analyzed similarly, with the exception that no MF analysis was conducted. For clinical observations, a case definition of two or more days with abnormal clinical observations was used to identify affected animals. Additionally, number and duration of abnormal clinical observations were evaluated utilizing the MF method. For pyrexis, animals with temperature values of 1 degree or greater above the baseline were identified as pyrexic for an individual day.

Data analyzed using the PF and Fisher's Exact methods, if not already dichotomous were dichotomized to a binary outcome (e.g. normal/abnormal) for each animal. Binary data was summarized by group via frequency distributions. In addition, for binary data, the relative risk (RR) was estimated and a 95% confidence interval (CI) calculated using the Cochran-Mantel-Haenszel method in SAS procedure PROC FREQ. The RR and associated CI were then translated to the PF scale (1−RR) for presentation. For the PROC FREQ analysis, stratification based on litter was utilized. Statistical significance was concluded if the 95% CI for the RR does not include 0. The MF method utilized a stratified bootstrap approach with the Highest Density Interval utilized to construct a 95% confidence interval for the MF based on the bootstrap distribution. Stratification was based on litter. Statistical significance was concluded if 0 was not in the confidence interval.

Viremia data were analyzed using a Generalized Friedman test (blocking on litter) to compare the group viremia distributions (quantitative) at each time point post-challenge. P-values smaller than 0.05 are considered statistically significant.

Pre-vaccination (Day −2) weight was analyzed using a linear mixed model with group (fixed effect), litter (random effect) and residual. Least-squares means were estimated and group comparisons were evaluated via P-values. Ninety-five percent Confidence Intervals were constructed as appropriate. Challenge Phase weights (Days 12, 21, 42) were analyzed using a linear mixed model with group, day and group by day interactions (fixed effects), challenge room and pen within challenge room (random effects) and an unstructured covariance representing the repeated measures on the animal level. Least-squares means were estimated and group comparisons were evaluated via P-values by study day. Average Daily Weight Gain was estimated and evaluated using a linear contrast of the fixed effect terms. Ninety-five percent Confidence Intervals were constructed as appropriate. P-values smaller than 0.05 are considered statistically significant.

The experimental inactivated baculovirus-expressed PCV3 ORF2 vaccine significantly prevented replicating virus being found in ILN, spleen, and lung, and significantly mitigated the severity of the amount of replicating PCV3 virus found in all tissues. The vaccine also numerically reduced mortality, clinical signs, gross lesions, and histologic lesions, in addition to a numerical increase in body weights following the challenge phase. These data demonstrates a clinically-relevant disease, correlating the clinical picture with evidence of replicating PCV3 virus in the tissues by RNAscope evaluation.

Two control pigs died during the challenge phase; no tentative diagnosis was suggested at necropsy of either pig. ILN, spleen and lung tissues from both pigs had moderate or severe evidence of the presence of PCV3, and kidney, heart, large intestine, and small intestine had PCV3 RNAscope scores of 1. In contrast, the three vaccinated pigs that died post-challenge had tentative diagnoses of bacterial septicemia or failure to thrive, which is common with young CDCD pigs. This diagnosis is supported by RNAscope results that were negative for all tissues, so the vaccinated pigs that died post-challenge are not considered mortalities due to PCV3.

Evaluating clinical signs, 14/25 (56%) control pigs had a clinical observation post-challenge compared to 11/23 (48%) vaccinated pigs. The limited occurrence of clinical signs is consistent with expectations from a laboratory evaluation of circovirus. Clinical observations during this study are similar to what are historically seen with the laboratory challenge model for PCV2.

This same trend was observed with body weights; least squares mean body weights were 0.92 kg heavier for vaccinates than controls at off-test, indicating better overall health (hydration and appetite). While up to 91% of the vaccinated pigs did become viremic, the load of virus (genomic copies) in the blood was significantly reduced by approximately a log in the vaccinates at each post-challenge time point (P≤0.0050; FIG. 28) when compared to controls. Few lesions were seen during gross evaluation at off-test and during histologic examination. The majority of the macroscopic lesions were enlarged lymph nodes (10/25 control pigs and 14/23 vaccinated pigs), and the majority of the microscopic lesions were mild lesions of the ILN.

RNAScope detects replicating virus in a tissue as opposed to immunohistochemistry or PCR which identifies genetic material of a virus whether it is replicating or dead. Strikingly, all control pigs had mild to moderate staining of the spleen and mild to severe staining of the ILN and lung. In contrast, six vaccinated pigs had no RNAScope staining in any tissue. All tissues from vaccinated animals demonstrated significant prevention of infection (by both prevented fraction and hypothesis testing analyses), and the ILN, spleen, and lung also demonstrated a reduction in severity by mitigated fraction.

Serology results were negative for all samples on all days. This may be due to the short window between vaccination and challenge.

The experimental inactivated baculovirus-expressed PCV3 ORF2 vaccine significantly prevented replicating virus being found in ILN, spleen, and lung, and significantly mitigated the severity of the amount of replicating PCV3 virus found in all tissues. The vaccine also numerically reduced mortality, clinical signs, gross lesions, and histologic lesions, in addition to a numerical increase in body weights following the challenge phase. This data demonstrates a clinically-relevant disease correlating the clinical picture with RNAscope evaluation and scoring. Taken altogether, the experimental baculovirus-expressed killed PCV3 ORF2 vaccine was shown to be efficacious against PCV3.

### Example 10

### Reproductive Study of PCV3 in Farrowing Sow

Vaccination phase: Forty-six pre-breeding gilt (≥5 months of age) were used in this study. All dams were screened to be free of viremia prior to vaccination by qPCR for the following agents: PCV3, PCV2, atypical porcine pestivirus (APPV), transmissible gastroenteritis virus (TGEV), porcine reproductive and respiratory syndrome virus (PRRSV) and porcine parvovirus (PPV). Animals were also shown to be seronegative for Influenza A and M. hyopneumoniae.

Gilts were divided into three treatment groups for this study: r=non-exposure and non-challenge to PCV3 (NTX), receipt of a placebo with challenge to PCV3, and vaccination with the PCV3 ORF2 vaccine with challenge to PCV3. Gilts were vaccinated on D0 and D21 based on their treatment groups (2 mL intramuscularly in the right neck). Gilts in the NTX treatment group were administered the placebo vaccine and housed separately from the gilts of the other treatment groups. Estrus synchronization was done by administration of MATRI™ (altrenogest) in their feed from day 17 to day 30. On day 30, P.G. 600® (serum gonadotropin [PMSG] and chorionic gonadotropin) was administered to all gilts. Animal were evaluated for estrus and bred between day 35-42. Thirty-six sows were confirmed pregnant on D77 (D35 of gestation) and used in this study (Table 150).

TABLE 150
Treatment groups and study design

| Treatment | | | | | |
|---|---|---|---|---|---|
| group | Treatment | n | Challenge | Farrowing | Necropsy |
| NTX | Placebo | 2 | None | D147-D159 | D168-D180 |
| Placebo | | 12 | D83 (~40 | litter data | (21 days Post |
| Vaccine | Vaccine | 19 | days of | & necropsy/ | farrowing) |
| | | | gestation) | tissue/blood | litter data |
| | | | PCV3 tissue | collection | & necropsy/ |
| | | | homogenate | | tissue/blood |
| | | | 2 mL each | | collection |
| | | | IM and IN | | |

Challenge phase: All animals in the placebo and vaccine treatment groups were challenged with PCV3 positive tissue homogenate 40 days into gestation. The PCV3 tissue homogenate was administered 2 mL each intramuscularly and intranasally to each animal. Keyhole limpet hemocyanin (KLH) emulsified in incomplete Freund's adjuvant (ICFA) containing 1 mg KLH/mL was administered two days before and two days after challenge. The tissue homogenate used for challenge was screened for extraneous agents by qPCR and deep sequencing.

Viremia: Serum was collected from sows throughout the study and was evaluated for viremia by qPCR (see Table 151 and FIG. 28). The bolded numbers in the top row of Table 151 indicate the respective day of the study. The unbolded numbers in Table 151 correspond to the measured number of genomic copies of PCV3/mL.

TABLE 151
Viremia of gilts challenged at D83 (40 days into gestation)

| Sow | Group | 0 | 13 | 21 | 28 | 34 | 69 | 76 | 82 | 96 | 103 | 110 | 117 | 124 | 131 | 138 |
|---|---|---|---|---|---|---|---|---|---|---|---|---|---|---|---|---|
| 104 | Vaccine | 0 | 0 | 0 | 0 | 0 | 0 | 0 | 0 | 0 | 0 | 0 | 0 | 0 | 0 | 0 |
| 108 | | 0 | 0 | 0 | 0 | 0 | 0 | 0 | 0 | 0 | 0 | 0 | 0 | 0 | 0 | 0 |
| 114 | | 0 | 0 | 0 | 0 | 0 | 0 | 0 | 0 | 0 | 0 | 0 | 0 | 0 | 0 | 0 |
| 116 | | 0 | 0 | 0 | 0 | 0 | 0 | 0 | 0 | 0 | 0 | 0 | 0 | 0 | 0 | 0 |
| 118 | | 0 | 0 | 0 | 0 | 0 | 0 | 0 | 0 | 0 | 0 | 0 | 0 | 0 | 0 | 0 |
| 120 | | 0 | 0 | 0 | 0 | 0 | 0 | 0 | 0 | 0 | 0 | 0 | 0 | 0 | 0 | 0 |
| 127 | | 0 | 0 | 0 | 0 | 0 | 0 | 0 | 0 | 0 | 0 | 0 | 0 | 0 | 0 | 0 |
| 132 | | 0 | 0 | 0 | 0 | 0 | 0 | 0 | 0 | 0 | 0 | 0 | 0 | 0 | 0 | 0 |
| 135 | | 0 | 0 | 0 | 0 | 0 | 0 | 0 | 0 | 0 | 0 | 0 | 0 | 0 | 0 | 0 |
| 136 | | 0 | 0 | 0 | 0 | 0 | 0 | 0 | 0 | 0 | 0 | 0 | 0 | 0 | 0 | 0 |
| 139 | | 0 | 0 | 0 | 0 | 0 | 0 | 0 | 0 | 0 | 0 | 0 | 0 | 0 | 0 | 0 |
| 142 | | 0 | 0 | 0 | 0 | 0 | 0 | 0 | 0 | 0 | 0 | 0 | 0 | 0 | 0 | 0 |
| 147 | | 0 | 0 | 0 | 0 | 0 | 0 | 0 | 0 | 0 | 0 | 35006 | 0 | 0 | 0 | 0 |
| 148 | | 0 | 0 | 0 | 0 | 0 | 0 | 0 | 0 | 0 | 0 | 0 | 0 | 0 | 0 | 0 |
| 152 | | 0 | 0 | 0 | 0 | 0 | 0 | 0 | 0 | 0 | 0 | 32676 | 0 | 0 | 0 | 0 |
| 153 | | 0 | 0 | 0 | 0 | 0 | 0 | 0 | 0 | 0 | 0 | 0 | 0 | 0 | 0 | 0 |
| 154 | | 0 | 0 | 0 | 0 | 0 | 0 | 0 | 0 | 0 | 0 | 0 | 0 | 0 | 0 | 0 |
| 158 | | 0 | 0 | 0 | 0 | 0 | 0 | 0 | 0 | 0 | 0 | 78456 | 0 | 0 | 0 | 0 |
| 159 | | 0 | 0 | 0 | 0 | 0 | 0 | 0 | 0 | 0 | 0 | 0 | 0 | 0 | 0 | 0 |
| 101 | NTX | 0 | 0 | 0 | 0 | 0 | 0 | 0 | 0 | 0 | 0 | 0 | 0 | 0 | 0 | 0 |
| 150 | | 0 | 0 | 0 | 0 | 0 | 0 | 0 | 0 | 0 | 0 | 37613 | 0 | 0 | 0 | 0 |
| 4 | Placebo | 0 | 0 | 0 | 0 | 0 | 0 | 0 | 0 | 0 | 125239 | 1648655 | 5091 | 461578 | 5554067 | 793015 |
| 103 | | 0 | 0 | 0 | 0 | 0 | 0 | 0 | 0 | 3893 | 1019339 | 133891 | 7911 | 651478 | 1046103 | 969657 |
| 125 | | 0 | 0 | 0 | 0 | 0 | 0 | 0 | 0 | 5278 | 518944 | 243141 | 1004485 | 2180344 | 690203 | 2100557 |
| 131 | | 0 | 0 | 0 | 0 | 0 | 0 | 0 | 0 | 17824 | 953267 | 121375 | 28287 | 2497839 | 1440941 | 1499740 |
| 137 | | 0 | 0 | 0 | 0 | 0 | 0 | 0 | 0 | 4778 | 45726 | 21069 | 2087 | 214688 | 249939 | 673275 |
| 138 | | 0 | 0 | 0 | 0 | 0 | 0 | 0 | 0 | 0 | 2940 | 6278 | 24441 | 990226 | 0 | 118748 |
| 144 | | 0 | 0 | 0 | 0 | 0 | 0 | 0 | 0 | 6545 | 320446 | 0 | 25158 | 469376 | 412792 | 1793810 |
| 149 | | 0 | 0 | 0 | 0 | 0 | 0 | 0 | 0 | 5025 | 647548 | 0 | 20126 | 2106547 | 2294852 | 9940749 |
| 151 | | 0 | 0 | 0 | 0 | 0 | 0 | 0 | 0 | 1535 | 3077 | 0 | 19658 | 3143481 | 913922 | 604511 |
| 155 | | 0 | 0 | 0 | 0 | 0 | 0 | 0 | 0 | 1717 | 0 | 0 | 34934 | 189094 | 119791 | 190931 |
| 156 | | 0 | 0 | 0 | 0 | 0 | 0 | 0 | 0 | 0 | 11605 | 19861 | 1331 | 314544 | 105099 | 336322 |
| 157 | | 0 | 0 | 0 | 0 | 0 | 0 | 0 | 0 | 2711 | 395432 | 20268 | 29523 | 868779 | 1688576 | 1485114 |

Three pigs in the vaccine group showed viremia at D110. The absence of viremia on D103 and D117 may indicate that this reading was either a false positive or a vaccine effect towards suppressing virus replication. Similarly, one of the two non-challenged NTX sows showed viremia at D110. The NTX animals were housed separately and the absence of viremia on D103 and D117 may indicate that these readings could be false positives. All of the placebo sows showed viremia after challenge and continued to have viremia until the day of farrow. Overall, the viremia data from sows indicates that the vaccine is able to abrogate virus replication in the sows.

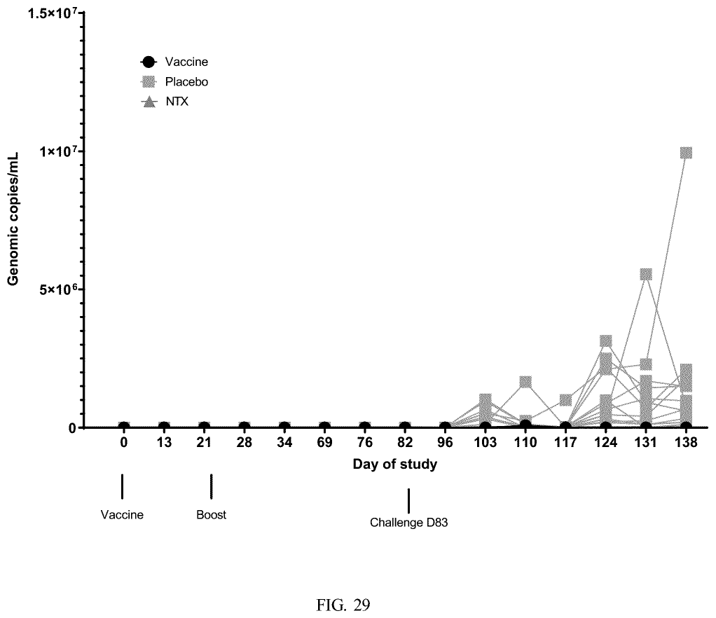

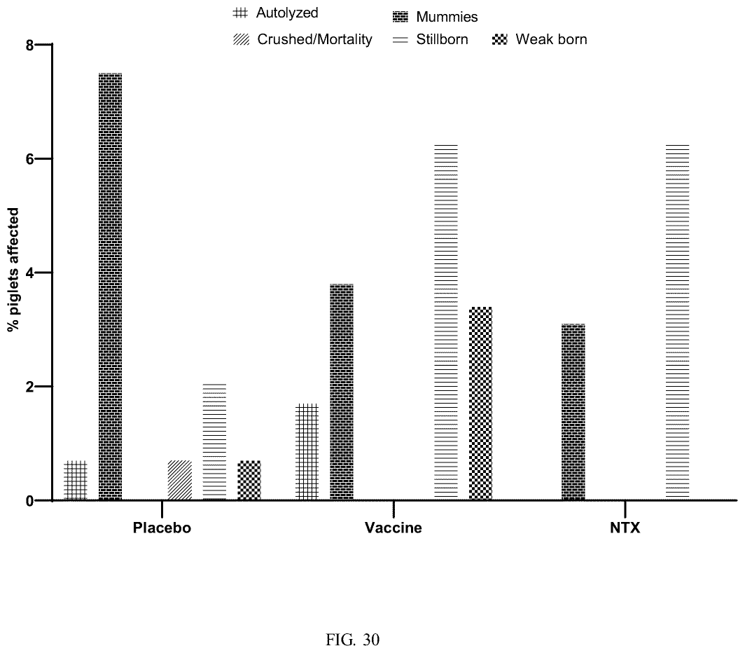

Clinical signs: All gilts were taken to farrowing. At farrow, the piglets were scored as healthy, mummies, weak-born, stillborn and autolysed. Any mortality arising from being crushed in the first three days after farrowing were also recorded. The percentage of affected mummies is shown in FIG. 30. One sow each in vaccine and placebo groups did not farrow.

According to FIG. 30, there is a clear reduction in the number of mummies in the vaccine group in comparison to the placebo group. One of the two sows in the NTX group had a single mummy and both sows had one stillborn piglet.

PCV3 is widely believed to be a reproductive disease. In the reproductive study, sows were vaccinated, boosted, and bred to evaluate the effect of a PCV3 challenge. The experimental inactivated baculovirus-expressed PCV3 ORF2 vaccine appears to almost completely abrogate virus replication in sows. Moreover, at farrow, vaccinated sows had just under 4% reduction in the number of mummies. This reduction could have a significant economic impact for swine producers.

### Example 11

### Preparation of PCV3 ORF2 Mutant in the FG Loop for Enhanced Virus-Like Particle Assembly

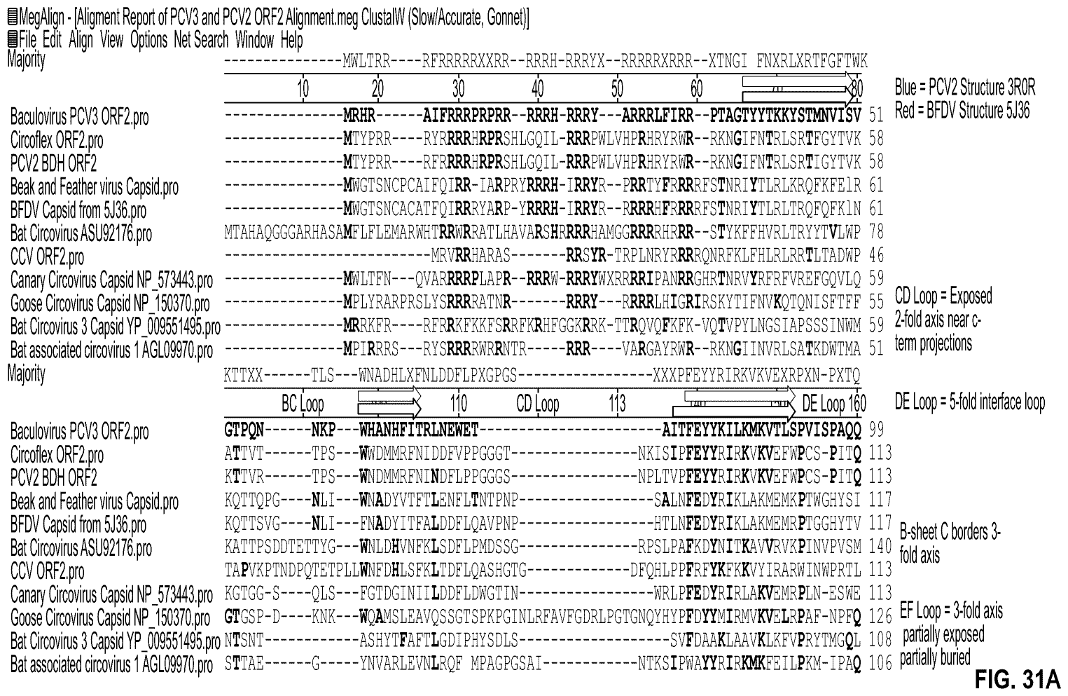

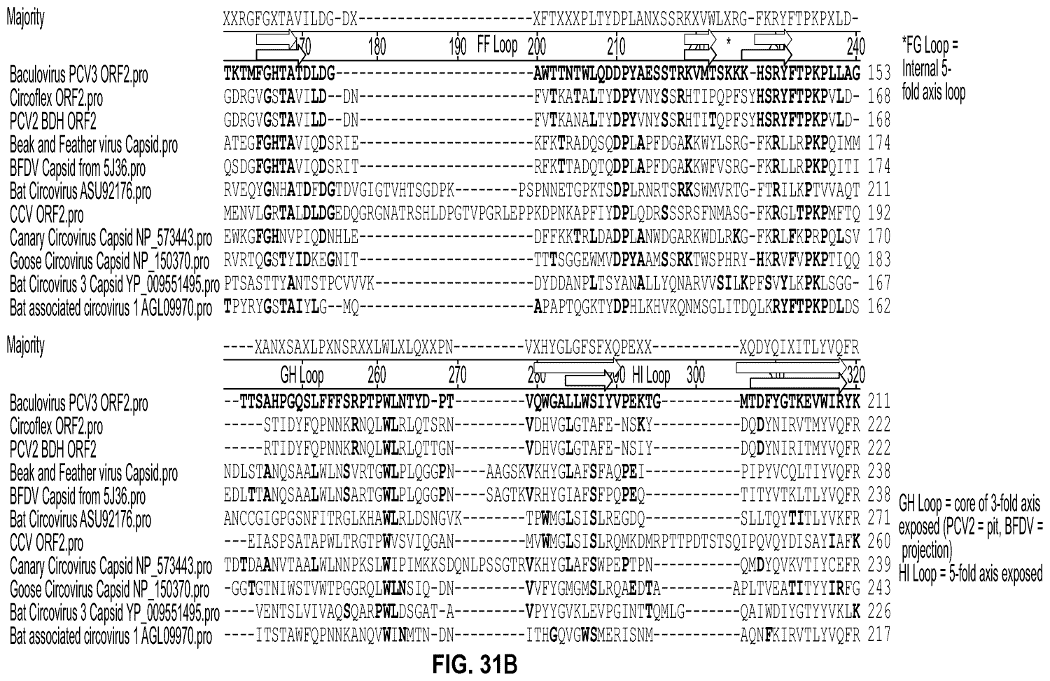

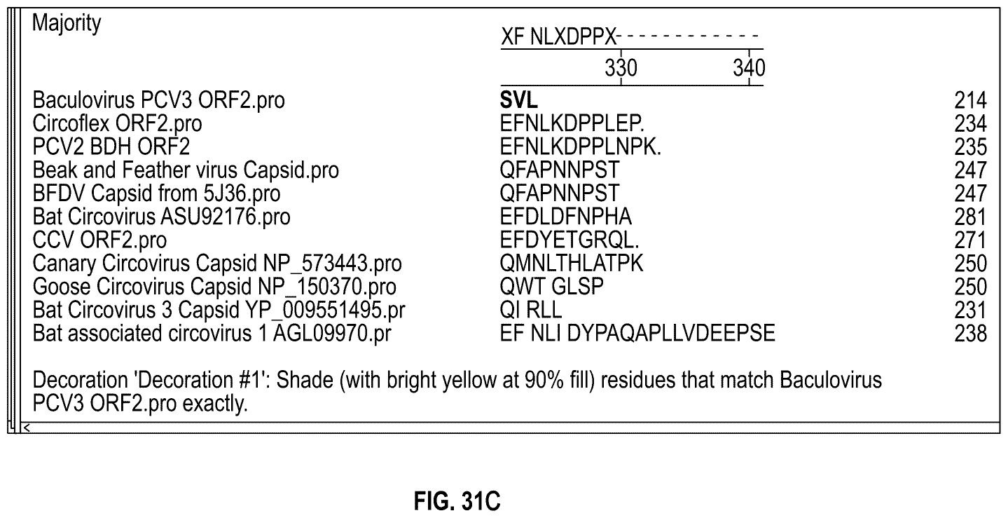

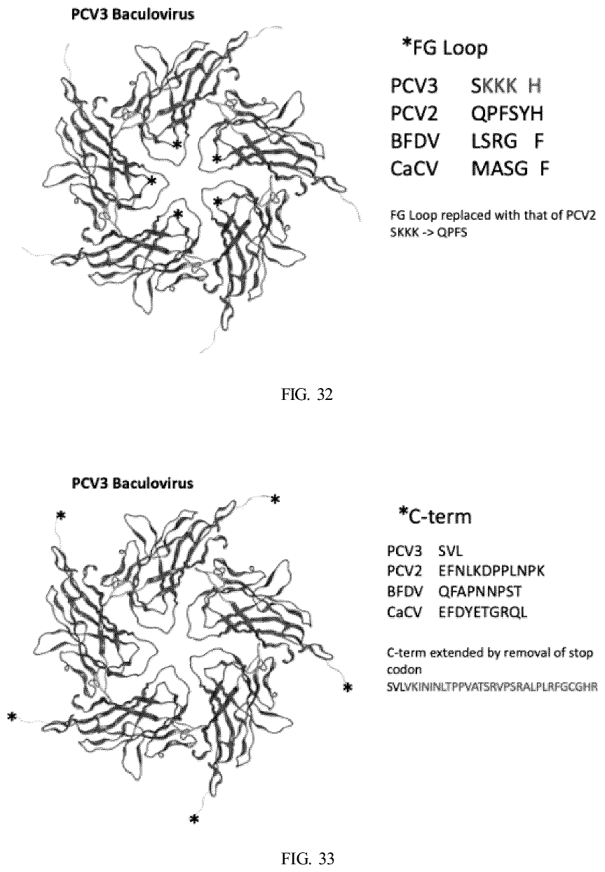

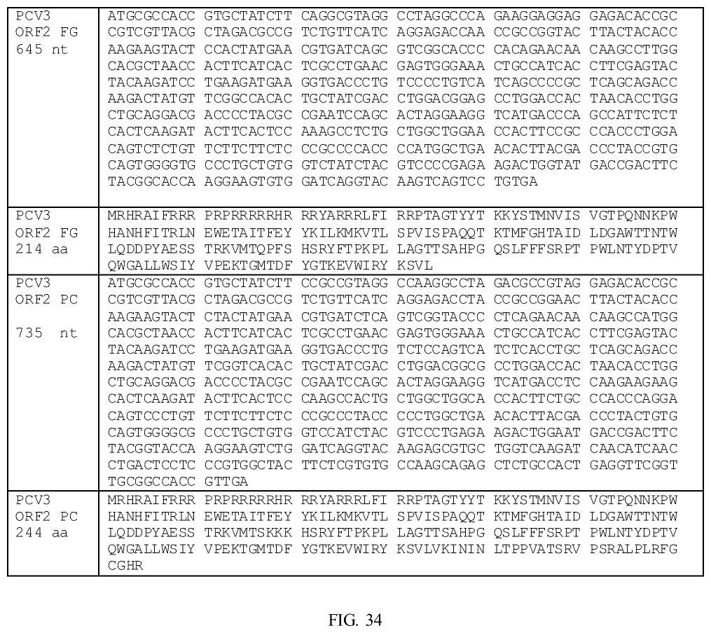

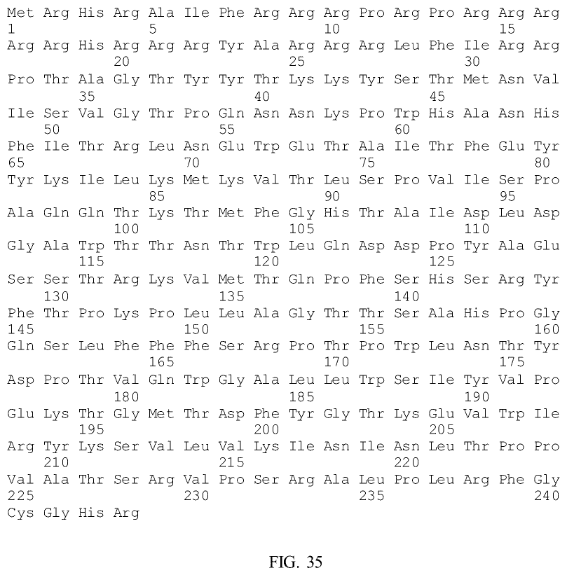

A number of Circovirus capsid sequences were aligned with Porcine circovirus type 3 (PCV3) capsid and two sequences for which structural data was available, Porcine circovirus type 2 (PCV2) capsid and Beak and feather disease virus (BFDV) capsid. Evaluation of the alignments with the structural data revealed that, despite the divergence of the capsid amino acid sequences between PCV2 and BFDV, the solved structures were very similar. This suggests that the structures of circovirus capsids may be similar despite their sequence divergence (FIGS. 31 and 32).

Additionally, the PCV3 capsid was the only aligned circovirus sequence that contained large amounts of positive charge in the FG loop which sits at the 5-fold interface of the PCV3 capsid. The large amount of positive charge in this region may result in repulsive forces without the presence of nucleic acid, as would be expected of virus-like particles (VLPs). Therefore, the lysines and histidine in this loop were mutated to the amino acids from PCV2 capsid (FIG. 32). This sequence was called PCV3 ORF2 FG (SEQ ID NO: 6).

The sequence was synthesized at Genscript and is cloned for recombinant baculoviruses for evaluation of PCV3 ORF2 expresssion and assembly into VLPs.

### Example 12

### Preparation of PCV3 ORF2 Mutant in the Native Stop Codon and Extension of the C-Terminus for Enhanced VLP Assembly

Evaluation of the alignments with the structural data described in Example 13 further disclosed that the PCV3 capsid had the shortest C-terminus sequence of any of the circovirus capsid sequences aligned. The C-terminus of PCV2 and BFDV capsid proteins project out away from the capsid. The short hydrophobic nature of the PCV3 capsid C-terminus would lead to the C-terminus being buried in the capsid and could lead to VLP instability without the presence of nucleic acid. Therefore, the native stop codon for the PCV3 capsid protein was mutated and the C-terminus was extended to the next stop codon in the virus sequence (FIG. 33). This sequence was called PCV3 ORF2 PC (SEQ ID NO: 7).

The sequence was synthesized at Genscript and is cloned for recombinant baculoviruses for evaluation of PCV3 ORF2 expresssion and assembly into VLPs.

### Example 13

Challenge Data from the Mutated PCV3 ORF2 Candidates in CDCD Pigs

Vaccination phase: Twenty-five Caesarian-derived colostrum-deprived (CDCD) pigs at approximately 3 weeks of age are vaccinated with either the enhanced expression baculovirus PCV3 ORF2 vaccine or a placebo matched control vaccine. Cord blood is collected from all pigs at delivery (C-section; D-22). Serum is separated and tested for PCV3 DNA and PCV2 DNA by PCR. On D-2, all pigs are bled for serum collection and then vaccinated with PCV2 Ingelvac CircoFLEX®.

Challenge phase: All animals are challenged with PCV3 positive tissue homogenate at 14 days post vaccination. Keyhole limpet hemocyanin (KLH) emulsified in incomplete Freund's adjuvant (ICFA) containing 1 mg KLH/1 mL is administered two days before and two days after challenge (Table 152). The tissue homogenate used for challenge is screened for extraneous agents by qPCR and deep sequencing. Animals are euthanized at day 42. At necropsy, a number of tissues including brain, heart, kidney, lung, spleen, large intestine, tonsil, tracheobronchial lymph node (TBLN), mesenteric lymph node (MLN), and external iliac lymph node (ILN) are collected.

TABLE 152
Study design

| Group | N | Vaccination | KLH/ICFA | Challenge | KLH/ICFA | Necropsy |
|---|---|---|---|---|---|---|
| Placebo | 25 | D0 | D12 | D14 | D16 | D42 |
| Vaccine | 25 | 2 mL IM (right | 2 mL IM | 1 mL IN/1 mL | 2 mL IM | |
| | | neck) | | IM | | |

### Laboratory Phase

Viremia: Post-challenge viremia in vaccinates and control animals is evaluated by qPCR. Following challenge, all control pigs are viremic. The viremia load (genomic copies/mL) is completely abrogated in the vaccinates.

Clinical signs: Virus replication in tissues of vaccinates and placebo animals post challenge is evaluated using RNAScope. RNAScope is a recently available technology that allows us to specifically tag and visualize viral mRNA. RNAScope detects replicating virus in a tissue as opposed to immunohistochemistry or PCR which identifies genetic material of a virus whether it is live or dead. Tissues are fixed and permeabilized to allow for target probe access sites of viral replication within the cells. A pair of PCV3 RNA specific oligonucleotide probes are then hybridized so as to sit within close proximity of each other on the PCV3 target RNA. (The detection of messenger RNA means the PCV3 virus is replicating, not just detection of PCV3 genetic material.) This is followed by the hybridization of a signal amplification molecule (SAM) that recognizes the pair of specific oligonucleotide probes. In non-specific reactions, the two probes do not sit next to each other preventing their hybridization with the SAM. The SAMs themselves are conjugated to an enzyme. As in in situ hybridization assays, the signals are detected using a chromogenic substrate followed by bright-field microscopic examination of slides. Slides for PCV3 RNAscope assay are stained and stained slides are read and scored (Table 153).

TABLE 153
RNAScope scoring scale

| Score | Description |
|---|---|
| 0 = normal | zero cells with PCV3 staining |
| 1 = mild | <10% of cells with PCV3 staining |
| 2 = moderate | 10-50% of cells with PCV3 staining |
| 3 = severe | >50% of cells with PCV3 staining |

No evidence of PCV3 replication is observed in any sections of cerebrum/cerebellum of any pig. Nearly all of the control pigs have at least mild PCV3 RNAscope staining in the kidney, heart, large intestine, and small intestine. Barring one pig, none of the other vaccinated pigs show any staining in the tissues evaluated.

The experimental inactivated enhanced expression baculovirus PCV3 ORF2 vaccine significantly prevents virus replication in ILN, spleen, and lung, and significantly mitigated the severity of the amount of replicating PCV3 virus found in all tissues. The vaccine also numerically reduces mortality, clinical signs, gross lesions, and histologic lesions, in addition to a numerical increase in body weights following the challenge phase. Taken altogether, the experimental enhanced expression baculovirus PCV3 ORF2 is shown to be efficacious against PCV3.

### Example 14

Challenge Data from the Mutated PCV3 ORF2 Candidates in Reproductive Model

Vaccination Phase: Forty-six pre-breeding gilt (≥5 months of age) are used in this study. All dams are screened at the Iowa State University Veterinary Diagnostic Laboratory (ISU-VDL) to be free of viremia prior to vaccination by qPCR for the following agents: PCV3, PCV2, atypical porcine pestivirus (APPV), transmissible gastroenteritis virus (TGEV), porcine reproductive and respiratory syndrome virus (PRRSV) and porcine parvovirus (PPV). Animals are also shown to be Seronegative for Influenza A and M. hyopneumoniae as confirmed by ISU-VDL.

Gilts are vaccinated on D0 and D21 based on their treatment groups. The NTX gilts are administered with the placebo vaccine. Estrus synchronization is done by administration of MATRI™ (altrenogest) in their feed from day 17 to day 30. On day 30, P.G. 600 is administered to all gilts. Animals are evaluated for estrus and bred between day 35-42. Thirty-six sows are confirmed pregnant on D77 (D35 of gestation) and used in this study (Table 154).

TABLE 154
Study design showing treatment groups

| Treatment | | | | | |
|---|---|---|---|---|---|
| group | Treatment | n | Challenge | Farrowing | Necropsy |
| NTX | Placebo | 5 | None | D147-D159 | D168-D180 |
| Placebo | | 15 | D83 (~40 | litter data | (21 days Post |
| Vaccine | Vaccine | 16 | days of | & necropsy/ | farrowing) |
| | | | gestation) | tissue/blood | litter data |
| | | | PCV3 tissue | collection | & necropsy/ |
| | | | homogenate | | tissue/blood |
| | | | 2 mL each | | collection |
| | | | IM and IN | | |

Challenge phase: All animals are challenged with PCV3 positive tissue homogenate 40 days into gestation. Keyhole limpet hemocyanin (KLH) emulsified in incomplete Freund's adjuvant (ICFA) containing 1 mg KLH/1 mL is administered two days before and two days after challenge. The tissue homogenate used for challenge was screened for extraneous agents by qPCR and deep sequencing.

### Laboratory Phase

Viremia: Serum is collected from sows throughout the study and is evaluated for viremia by qPCR.

None of the vaccinated gilts show viremia on any data point samples post challenge. All NTX and Placebo gilts show viremia starting on week post challenge and continuing to show viremia for 3-5 weeks post challenge. Overall, the viremia data from sows indicates that the vaccine is able to abrogate virus replication in the sows.

Clinical signs: All gilts are taken to farrowing. At farrow, the piglets are scored as healthy, mummies, weak-born, stillborn and autolysed. Any mortality arising from being crushed in the first three days after farrowing are also recorded.

There is a clear and significant reduction in the total number of mummies in the vaccine group in comparison to the placebo group. A similar reduction in stillborn and weak born piglets is observed in the vaccine group as compared to the placebo and NTX group. Additionally, RNAScope data from the tissues indicate a significant reduction to complete abrogation of viral replication in tissues of vaccinated sows and piglets when compared to tissue from NTX and Placebo groups.

PCV3 is widely believed to be a reproductive disease. In the reproductive model, sows are vaccinated, boosted, and bred to evaluate the effect of a PCV3 challenge. Vaccinates (sows and piglets at thereof) also show a reduction or elimination of virus replication in examined tissues. The experimental inactivated enhanced expression baculovirus PCV3 ORF2 vaccine appears to almost completely abrogate virus replication in sows. Moreover, at farrow, vaccinated sows had a significant reduction in the number of mummies. This reduction could have a significant economic impact for swine producers.

PCV3 in Combination with PPV

Reference is made to WO2018/083156, the disclosure of which is incorporated by reference.

The objective of this study is to evaluate the onset of immunity for the herein disclosed PCV3 vaccine (investigational vaccine product 1 (termed “IVP1” in the following)) when used in association with a subunit porcine parvovirus vaccine as described in the Examples, in particular produced according to Examples 1 and 2, of WO2018/083156 (termed “IVP2” in the following), and wherein this mixture of IVP1 and IVP2 is also named “IVP2/IVP1” hereinafter.

This study includes 60 cesarean-derived, colostrum deprived (CDCD) pigs that are seronegative for PCV3 and PPV, of which 30 are vaccinated with the mixture IVP2/IVP1 and 30 (the control group) receive a sterile diluent (water for injection) at 3 weeks of age (i.e., on study day 0 (D0)), followed by a virulent challenge of PCV3 on D14.

Vaccination with IVP2/IVP1 results in a significant increase in pigs positive for PCV3 serology, viremia and RNAscope. By D42, all pigs of the IVP2/IVP1 group are serologically positive for PVC3, while in the control group significantly less pigs are positive.

Upon assessment of the primary outcome parameters, the vaccination with IVP2/IVP1 significantly reduces and/or abrogates viremia in vaccinates. Furthermore, the overall level of the histologic lesions as determined by H&E staining is more severe in the control group with significantly more pigs having moderate to severe scores in at least one category of lesion evaluation, whereas a considerable less number of the vaccinated pigs have a moderate lesion score, with none being severe. More importantly, on a histological level as determined by virus specific RNAScope staining, the vaccine is able to prevent or reduce viral replication in tissues including but not limited to heart, kidney, lung, intestine and neural tissue.

In conclusion, IVP1 used in association with IVP2 provides efficacious active immunization of 3 week old CDCD pigs when challenged with virulent PCV3 on day 14post vaccination.

### Example 16

PCV3 in Combination with PPV and PRRSV

Reference is made to WO2018/083156 and WO2012/110489, the disclosure of which is incorporated by reference.

The objective of this vaccination-challenge study is to provide data on the associated use of the herein disclosed PCV3 vaccine and the above described (in Example 15, with reference to WO2018/083156) parvovirus subunit vaccine IVP2 (IVP1/IVP2 as described above) with a PRRS MLV vaccine (said PRRS MLV deposited with European Collection of Cell Cultures (ECACC) under the Accession Number ECACC 1 1012502) described in the Examples of WO2012/110489 (termed “IVP3”, and the mixture is termed “IVP1/IVP2/IVP3” in the following) in 5- to 6-month-old gilts.

Twenty-seven gilts originate from a herd previously tested negative for PCV3 with no prior PCV3 history of disease or vaccination. Gilts are randomized into 3 treatment groups of n=9 receiving vaccination on D0 and boostered on D21: T1 Negative Control, T2 IVP1/IVP2/IVP3, T3 non-treated control gilts (NTX) with each group housed separately.

Gilts are vaccinated, bred and become pregnant. At approximately 40 days of gestation (dG), all gilts are inoculated with the PCV3 challenge strain (as herein described). Gilts are bled weekly except during synchronization and breeding (D35-D70), and sera are tested.

Gilts are allowed to farrow and the litters are examined for mummies, stillborn and weak born piglets. Overall, vaccinated gilts and sows show none to a significantly lesser number of mummies, stillborn and weak born litters when compared to controls or NTX groups

When examined for viremia, contrary to control groups, T2 gilts show complete abrogation of viremia post challenge. In conclusion, the combination vaccine IVP1/IVP2/IVP3 is efficacious in preventing viremia and PCV3 infection of fetuses at 40dG.

Histologically, T2 gilts are able to prevent viral replication in key tissues post-challenge. Significant virus relication and thereby clinical manifestation of PCV3 is observed in control gilts/sows and litters. This is visualized by using H&E staining and virus RNAScope assay that detects replication virual mRNA in cells and tissues. In conclusion, the combination vaccine IVP1/IVP2/IVP3 is efficacious in preventing clinical signs PCV3 infection of gilts, sows and fetuses at 40dG.

### ***

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

1-141. (canceled)

142. A method of preventing PCV3 viremia in a piglet, wherein the method comprises administering a recombinant PCV3 ORF2 protein to a pregnant gilt or pregnant sow, and wherein the pregnant gilt or pregnant sow gives birth to the piglet.

143. The method of claim 142 wherein the sow suckles the piglet.

144. The method of claim 143 wherein the recombinant PCV3 ORF3 protein is in a composition, wherein the composition further comprises a veterinary-acceptable carrier selected from the group consisting of a solvent, a dispersion media, a coating, a stabilizing agent, a diluent, a preservative, an anti-microbial agent, an antifungal agent, an isotonic agent, an adsorption delaying agent, an adjuvant, cell culture supernatant, a stabilizing agent, a viral vector, an expression vector, and an immunomodulatory agent, or any combination thereof.

145. The method of claim 144 wherein the recombinant PCV3 ORF2 protein in the composition further comprises an amino acid sequence having at least 97% sequence identity with SEQ ID NO:4.

146. The method of claim 145 wherein the PCV3 ORF2 protein in the composition is a recombinant baculovirus expressed PCV3 ORF2 protein.

147. The method of administering the composition of claim 146 to the pregnant gilt or pregnant sow comprising a multi-dose regimen.

148. The method of administering the composition of claim 147 to the pregnant gilt or pregnant sow comprising a two-dose regimen of the composition.

149. A method of preventing PCV3 viremia in a piglet, wherein the method comprises administering a recombinant PCV3 ORF2 protein to a gilt or sow before pregnancy, wherein the gilt or sow becomes pregnant with the piglet, and wherein the pregnant gilt or sow gives birth to the piglet.

150. The method of claim 149, wherein the sow suckles the piglet.

151. The method of claim 149 wherein the recombinant PCV3 ORF2 protein is in a composition, wherein the composition further comprises a veterinary-acceptable carrier selected from the group consisting of a solvent, a dispersion media, a coating, a stabilizing agent, a diluent, a preservative, an anti-microbial agent, an antifungal agent, an isotonic agent, an adsorption delaying agent, an adjuvant, cell culture supernatant, a stabilizing agent, a viral vector, an expression vector, and an immunomodulatory agent, or any combination thereof.

152. The method of claim 151 wherein the composition further comprises an amino acid sequence having at least 97% sequence identity with SEQ ID NO:4.

153. The method of claim 152 wherein the PCV3 ORF2 protein is a recombinant baculovirus expressed PCV3 ORF2 protein.

154. The method of administering the composition of claim 153 to the pregnant gilt or pregnant sow comprising a multi-dose regimen.

155. The method of administering the composition of claim 154 to the pregnant gilt or pregnant sow comprising a two-dose regimen of the composition.

156. A method of preventing PCV3 viremia in a piglet, wherein the method comprises administering a recombinant PCV3 ORF2 protein to a gilt or sow before mating/insemination of the gilt or sow.

157. The method of claim 156, wherein the sow suckles the piglet.

158. The method of claim 156 wherein the recombinant PCV3 ORF2 protein is in a composition, wherein the composition further comprises a veterinary-acceptable carrier selected from the group consisting of a solvent, a dispersion media, a coating, a stabilizing agent, a diluent, a preservative, an anti-microbial agent, an antifungal agent, an isotonic agent, an adsorption delaying agent, an adjuvant, cell culture supernatant, a stabilizing agent, a viral vector, an expression vector, and an immunomodulatory agent, or any combination thereof.

159. The method of claim 158 wherein the recombinant PCV3 ORF2 protein in the composition further comprises an amino acid sequence having at least 97% sequence identity with SEQ ID NO:4.

160. The method of claim 159 wherein the PCV3 ORF2 protein in the composition is a recombinant baculovirus expressed PCV3 ORF2 protein.

161. The method of administering the composition of claim 160 to the pregnant gilt or pregnant sow comprising a multi-dose regimen.

162. The method of administering the composition of claim 161 to the pregnant gilt or pregnant sow comprising a two-dose regimen of the composition.

163. A method of preventing PCV3 viremia in a piglet, wherein the method comprises administering a recombinant PCV3 ORF2 protein to a pregnant gilt or pregnant sow, wherein the piglet is born by the sow; and wherein the piglet suckles from the sow.


SEQUENCE LISTING (37 sequences)

SEQ ID NO: 1 - DNA, 645 bp, Porcine circovirus 3
  source 1..645: mol_type = unassigned DNA
atgagacacagagctatattcagaagaagaccccgcccaaggagacgacgacgccacagaaggcgctatgccagaagacgactattcattaggaggcccacagctggcacatactacacaaagaaatactccacaatgaacgtcatatccgttggaacccctcagaataacaagccctggcacgccaaccacttcattacccgcctaaacgaatgggaaactgcaattacctttgaatattataagatactaaaaatgaaagttacactcagccctgtaatttctccggctcagcaaacaaaaactatgttcgggcacacagccatagatctagacggcgcctggaccacaaacacttggctccaagacgacccttatgcggaaagttccactcgtaaagttatgacttctaaaaaaaaacacagccgttacttcacccccaaaccacttctggcgggaactaccagcgctcacccaggacaaagcctcttctttttctccagacccaccccatggctcaacacatatgaccccaccgttcaatggggagcactgctttggagcatttatgtcccggaaaaaactggaatgacagacttctacggcaccaaagaagtttggattcgttacaagtccgttctctga

SEQ ID NO: 2 - DNA, 134448 bp, Porcine circovirus 3
  source 1..134448: mol_type = unassigned DNA
gaattctacccgtaaagcgagtttagttttgaaaaacaaatgacatcatttgtataatgacatcatcccctgattgtgttttacaagtagaattctatccgtaaagcgagttcagttttgaaaacaaatgagtcatacctaaacacgttaataatcttctgatatcagcttatgactcaagttatgagccgtgtgcaaaacatgagataagtttatgacatcatccactgatcgtgcgttacaagtagaattctactcgtaaagccagttcggttatgagccgtgtgcaaaacatgacatcagcttatgactcatacttgattgtgttttacgcgtagaattctactcgtaaagcgagttcggttatgagccgtgtgcaaaacatgacatcagcttatgagtcataattaatcgtgcgttacaagtagaattctactcgtaaagcgagttgaaggatcatatttagttgcgtttatgagataagattgaaagcacgtgtaaaatgtttcccgcgcgttggcacaactatttacaatgcggccaagttataaaagattctaatctgatatgttttaaaacacctttgcggcccgagttgtttgcgtacgtgactagcgaagaagatgtgtggaccgcagaacagatagtaaaacaaaaccctagtattggagcaataatcgatttaaccaacacgtctaaatattatgatggtgtgcattttttgcgggcgggcctgttatacaaaaaaattcaagtacctggccagactttgccgcctgaaagcatagttcaagaatttattgacacggtaaaagaatttacagaaaagtgtcccggcatgttggtgggcgtgcactgcacacacggtattaatcgcaccggttacatggtgtgcagatatttaatgcacaccctgggtattgcgccgcaggaagccatagatagattcgaaaaagccagaggtcacaaaattgaaagacaaaattacgttcaagatttattaatttaattaatattatttgcattctttaacaaatactttatcctattttcaaattgttgcgcttcttccagcgaaccaaaactatgcttcgcttgctccgtttagcttgtagccgatcagtggcgttgttccaatcgacggtaggattaggccggatattctccaccacaatgttggcaacgttgatgttacgtttatgcttttggttttccacgtacgtcttttggccggtaatagccgtaaacgtagtgccgtcgcgcgtcacgcacaacaccggatgtttgcgcttgtccgcggggtattgaaccgcgcgatccgacaaatccaccactttggcaactaaatcggtgacctgcgcgtcttttttctgcattatttcgtctttcttttgcatggtttcctggaagccggtgtacatgcggtttagatcagtcatgacgcgcgtgacctgcaaatctttggcctcgatctgcttgtccttgatggcaacgatgcgttcaataaactcttgttttttaacaagttcctcggttttttgcgccaccaccgcttgcagcgcgtttgtgtgctcggtgaatgtcgcaatcagcttagtcaccaactgtttgctctcctcctcccgttgtttgatcgcgggatcgtacttgccggtgcagagcacttgaggaattacttcttctaaaagccattcttgtaattctatggcgtaaggcaatttggacttcataatcagctgaatcacgccggatttagtaatgagcactgtatgcggctgcaaatacagcgggtcgccccttttcacgacgctgttagaggtagggcccccattttggatggtctgctcaaataacgatttgtatttattgtctacatgaacacgtatagctttatcacaaactgtatattttaaactgttagcgacgtccttggccacgaaccggacctgttggtcgcgctctagcacgtaccgcaggttgaacgtatcttctccaaatttaaattctccaattttaacgcgagccattttgatacacgtgtgtcgattttgcaacaactattgttttttaacgcaaactaaacttattgtggtaagcaataattaaatatgggggaacatgcgccgctacaacactcgtcgttatgaacgcagacggcgccggtctcggcgcaagcggctaaaacgtgttgcgcgttcaacgcggcaaacatcgcaaaagccaatagtacagttttgatttgcatattaacggcgattttttaaattatcttatttaataaatagttatgacgcctacaactccccgcccgcgttgactcgctgcacctcgagcagttcgttgacgccttcctccgtgtggccgaacacgtcgagcgggtggtcgatgaccagcggcgtgccgcacgcgacgcacaagtatctgtacaccgaatgatcgtcgggcgaaggcacgtcggcctccaagtggcaatattggcaaattcgaaaatatatacagttgggttgtttgcgcatatctatcgtggcgttgggcatgtacgtccgaacgttgatttgcatgcaagccgaaattaaatcattgcgattagtgcgattaaaacgttgtacatcctcgcttttaatcatgccgtcgattaaatcgcgcaatcgagtcaagtgatcaaagtgtggaataatgttttctttgtattcccgagtcaagcgcagcgcgtattttaacaaactagccatcttgtaagttagtttcatttaatgcaactttatccaataatatattatgtatcgcacgtcaagaattaacaatgcgcccgttgtcgcatctcaacacgactatgatagagatcaaataaagcgcgaattaaatagcttgcgacgcaacgtgcacgatctgtgcacgcgttccggcacgagctttgattgtaataagtttttacgaagcgatgacatgacccccgtagtgacaacgatcacgcccaaaagaactgccgactacaaaattaccgagtatgtcggtgacgttaaaactattaagccatccaatcgaccgttagtcgaatcaggaccgctggtgcgagaagccgcgaagtatggcgaatgcatcgtataacgtgtggagtccgctcattagagcgtcatgtttagacaagaaagctacatatttaattgatcccgatgattttattgataaattgaccctaactccatacacggtattctacaatggcggggttttggtcaaaatttccggactgcgattgtacatgctgttaacggctccgcccactattaatgaaattaaaaattccaattttaaaaaacgcagcaagagaaacatttgtatgaaagaatgcgtagaaggaaagaaaaatgtcgtcgacatgctgaacaacaagattaatatgcctccgtgtataaaaaaaatattgaacgatttgaaagaaaacaatgtaccgcgcggcggtatgtacaggaagaggtttatactaaactgttacattgcaaacgtggtttcgtgtgccaagtgtgaaaaccgatgtttaatcaaggctctgacgcatttctacaaccacgactccaagtgtgtgggtgaagtcatgcatcttttaatcaaatcccaagatgtgtataaaccaccaaactgccaaaaaatgaaaactgtcgacaagctctgtccgtttgctggcaactgcaagggtctcaatcctatttgtaattattgaataataaaacaattataaatgtcaaatttgttttttattaacgatacaaaccaaacgcaacaagaacatttgtagtattatctataattgaaaacgcgtagttataatcgctgaggtaatatttaaaatcattttcaaatgattcacagttaatttgcgacaatataattttattttcacataaactagacgccttgtcgtcttcttcttcgtattccttctctttttcatttttctcctcataaaaattaacatagttattatcgtatccatatatgtatctatcgtatagagtaaattttttgttgtcataaatatatatgtcttttttaatggggtgtatagtaccgctgcgcatagtttttctgtaatttacaacagtgctattttctggtagttcttcggagtgtgttgctttaattattaaatttatataatcaatgaatttgggatcgtcggttttgtacaatatgttgccggcatagtacgcagcttcttctagttcaattacaccattttttagcagcaccggattaacataactttccaaaatgttgtacgaaccgttaaacaaaaacagttcacctcccttttctatactattgtctgcgagcagttgtttgttgttaaaaataacagccattgtaatgagacgcacaaactaatatcacaaactggaaatgtctatcaatatatagttgctgatatcatggagataattaaaatgataaccatctcgcaaataaataagtattttactgttttcgtaacagttttgtaataaaaaaacctataaatattccggattattcataccgtcccaccatcgggcgcggatccgccaccatgagacacagagctatattcagaagaagaccccgcccaaggagacgacgacgccacagaaggcgctatgccagaagacgactattcattaggaggcccacagctggcacatactacacaaagaaatactccacaatgaacgtcatatccgttggaacccctcagaataacaagccctggcacgccaaccacttcattacccgcctaaacgaatgggaaactgcaattacctttgaatattataagatactaaaaatgaaagttacactcagccctgtaatttctccggctcagcaaacaaaaactatgttcgggcacacagccatagatctagacggcgcctggaccacaaacacttggctccaagacgacccttatgcggaaagttccactcgtaaagttatgacttctaaaaaaaaacacagccgttacttcacccccaaaccacttctggcgggaactaccagcgctcacccaggacaaagcctcttctttttctccagacccaccccatggctcaacacatatgaccccaccgttcaatggggagcactgctttggagcatttatgtcccggaaaaaactggaatgacagacttctacggcaccaaagaagtttggattcgttacaagtccgttctctgagcggccgctgcagatctgatcctttcctgggacccggcaagaaccaaaaactcactctcttcaaggaaatccgtaatgttaaacccgacacgatgaagcttgtcgttggatggaaaggaaaagagttctacagggaaacttggacccgcttcatggaagacagcttccccattgttaacgaccaagaagtgatggatgttttccttgttgtcaacatgcgtcccactagacccaaccgttgttacaaattcctggcccaacacgctctgcgttgcgaccccgactatgtacctcatgacgtgattaggatcgtcgagccttcatgggtgggcagcaacaacgagtaccgcatcagcctggctaagaagggcggcggctgcccaataatgaaccttcactctgagtacaccaactcgttcgaacagttcatcgatcgtgtcatctgggagaacttctacaagcccatcgtttacatcggtaccgactctgctgaagaggaggaaattctccttgaagtttccctggtgttcaaagtaaaggagtttgcaccagacgcacctctgttcactggtccggcgtattaaaacacgatacattgttattagtacatttattaagcgctagattctgtgcgttgttgatttacagacaattgttgtacgtattttaataattcattaaatttataatctttagggtggtatgttagagcgaaaatcaaatgattttcagcgtctttatatctgaatttaaatattaaatcctcaatagatttgtaaaataggtttcgattagtttcaaacaagggttgtttttccgaaccgatggctggactatctaatggattttcgctcaacgccacaaaacttgccaaatcttgtagcagcaatctagctttgtcgatattcgtttgtgttttgttttgtaataaaggttcgacgtcgttcaaaatattatgcgcttttgtatttctttcatcactgtcgttagtgtacaattgactcgacgtaaacacgttaaataaagcttggacatatttaacatcgggcgtgttagctttattaggccgattatcgtcgtcgtcccaaccctcgtcgttagaagttgcttccgaagacgattttgccatagccacacgacgcctattaattgtgtcggctaacacgtccgcgatcaaatttgtagttgagctttttggaattatttctgattgcgggcgtttttgggcgggtttcaatctaactgtgcccgattttaattcagacaacacgttagaaagcgatggtgcaggcggtggtaacatttcagacggcaaatctactaatggcggcggtggtggagctgatgataaatctaccatcggtggaggcgcaggcggggctggcggcggaggcggaggcggaggtggtggcggtgatgcagacggcggtttaggctcaaatgtctctttaggcaacacagtcggcacctcaactattgtactggtttcgggcgccgtttttggtttgaccggtctgagacgagtgcgatttttttcgtttctaatagcttccaacaattgttgtctgtcgtctaaaggtgcagcgggttgaggttccgtcggcattggtggagcgggcggcaattcagacatcgatggtggtggtggtggtggaggcgctggaatgttaggcacgggagaaggtggtggcggcggtgccgccggtataatttgttctggtttagtttgttcgcgcacgattgtgggcaccggcgcaggcgccgctggctgcacaacggaaggtcgtctgcttcgaggcagcgcttggggtggtggcaattcaatattataattggaatacaaatcgtaaaaatctgctataagcattgtaatttcgctatcgtttaccgtgccgatatttaacaaccgctcaatgtaagcaattgtattgtaaagagattgtctcaagctcggatcccgcacgccgataacaagccttttcatttttactacagcattgtagtggcgagacacttcgctgtcgtcgacgtacatgtatgctttgttgtcaaaaacgtcgttggcaagctttaaaatatttaaaagaacatctctgttcagcaccactgtgttgtcgtaaatgttgtttttgataatttgcgcttccgcagtatcgacacgttcaaaaaattgatgcgcatcaattttgttgttcctattattgaataaataagattgtacagattcatatctacgattcgtcatggccaccacaaatgctacgctgcaaacgctggtacaattttacgaaaactgcaaaaacgtcaaaactcggtataaaataatcaacgggcgctttggcaaaatatctattttatcgcacaagcccactagcaaattgtatttgcagaaaacaatttcggcgcacaattttaacgctgacgaaataaaagttcaccagttaatgagcgaccacccaaattttataaaaatctattttaatcacggttccatcaacaaccaagtgatcgtgatggactacattgactgtcccgatttatttgaaacactacaaattaaaggcgagctttcgtaccaacttgttagcaatattattagacagctgtgtgaagcgctcaacgatttgcacaagcacaatttcatacacaacgacataaaactcgaaaatgtcttatatttcgaagcacttgatcgcgtgtatgtttgcgattacggattgtgcaaacacgaaaactcacttagcgtgcacgacggcacgttggagtattttagtccggaaaaaattcgacaccacaactatgcacgttcgtttgactggtacgccgtcggcgtgttaacatacaagttgctaaccggcggccgacacccatttgaaaaaagcgaagacgaaatgttggacttgaatagcatgaagcgtcgtcagcaatacaatgacattggcgttttaaaacacgttcgtaacgttaacgctcgtgactttgtgtactgcctaacaagatacaacatagattgtagactcacaaattacaaacaaattataaaacatgagtttttgtcgtaaaaatgccacttgttttacgagtagaattctacgtgtaacacacgatctaaaagatgatgtcattttttatcaatgactcatttgttttaaaacagacttgttttacgagtagaattctacgtgtaaagcatgatcgtgagtggtgttaataaaatcataaaaattattgtaaatgtttattatttaaaaacgattcaaatatataataaaaacaatctacatctatttcttcacaatccataacacacaacaggtccatcaatgagtttttgtctttatccgacatactatgtgcatgtaacaaatcaaatacatcttttaaatttttatacacatctttacattgtctaccaaaatctttaataaccctataacaaggaaaagacttttcttcttgcgtggttttgccgcgcagatattgaaataaaatgtgcatgcacgacaacttgtgtttactaaaatgctccttgcctataccgcaaaaccggccatacatttcggcgattacacgcggacaattgtacgattcgtctacgtgtaaacgatcatcataatcactcttgcgcaaacgaataaattttttcaccgcttccgacaaacgaggcaccaattcggcgggcacgcttcgatacattattctgtgcacataagttaccacacaaaatttattgtaccaccatccgacaacgtcgttattagggttgaacacgttggcgatgcgcagcagtttcccgtttctcatgaaatattcaaagcggcccaaaataatttgcaagcaatccaacatgtcttgagaaatttctcgttcaaaattgttcaaagagaatatctgccatccgttttgaacgcgcacgctgacgggaaccaccgcatcgatttgctccaacacttcacggacgttatcgtcgatgcccatcgtttcgctggtgctgaaccaatgggaaaggctcttgatggaatcgcccgcgtctatcatcttgaccgcttcgtcaaaggtgcaactgccgctcttcaaacgccgcatagcggtcacgtcccgctctatgcacgacataccgtttacgtacgattctgataggtattcctgaactatacggtaatggtgatacgactcgccatacacgtcgtgcacctcattgtatttagcataataattgtaaattattaactttgcagcgagagacatgttgtcagtaaagcggtgctaggctcaataatactgatgtacaggcacgcgtgctatttatatataatttcgcaaggaggggagctgttatcggttgctattattaaagaatggccgtctgtttttatcacaagcttggcagcctcaaccatgaagcgtcgtcattgtaaattaaattctctgcctcaagaattatttgacaagattgtcgagtatttatctttatctgattactgcaatttggtgcttgtctgtaaaagaccttctagtaaatataacgtgatatttgatagtactaatcaccaacatttgaaaggcgtgtacaaaaagacagacgtgcaaataacaagctacaacgaatacatcaactgtatttgcaacgaactgagacaagacgaattctatgccaaatcatcatggattgcgagtatttgcggtcaccagagagcgacaatttttagtgtaacaaataaacaagtagaaatgaaatatcatttgtataatatagcaattgtggaaagtgaagattgcaacggattttacccatttgagccaacgcgcgattgtttaatatgcaaacaaaaaaaccaatgtcctcgtaattcatttattgtttcgttgtgtaaatatttagaaaaacaaaatgtacaatcaaactttatatattatttatacgaaataaatacataataataactattatacatgtttttattttacaatacttcctgtataacctctctaactacattaggagtacaatccacgtcaattacacgtttagctatttttctaattttgtaatgtttatcgtagagtttttcgttaatacattgaatagccaacaagggatttgggtgcacaccgtcatagagtacttccatgtcgtcttcaaagcgcatttttcgcttgcgaaaatgccgctcttggcccaaaacaaaagcgagtttgatgcggtcgtcgatgcgttccgaaaatacggccaaatgctggtgtttggtgatgtcgcgcggaaacgtcaccgtgccatttttgctttccgccacgacggcggttttcaatttttcggccgactgcagcatgttaagtttggcgtcgagttcgtgcaaacgcaattcaaactgctcaaacctgttgcccacctcgttcttgaacgtctcgtgggtgaccataaatttttcgctgtttgcattcagtttctttacatgttttaaaacagattcaatcttgtcgcgcaaatcatcacgctcgccttcagtttgaatgtgcagcaacgcgttgcttttgttggcaaaatttaaccgcatcaaaatttccaacaacccgtgcttggtcgcgaacaatgcgcccaacgagttgagatcgcgtttggatctctgtttgtgaaaaacaatttcgtttaaatggtaaacttgatcgccgtcccaattgcaatcaagtatgtcgtcgtgcgcaatttcaagacctttgcaaaaatctatcacattgtagcattttgcgttcgtgtcgctgtgcacgtatctgtacttgaaactgtgcgtgttgcatttgaatgagtcccatttaacgatgtgcgaccattgttgggcgtttatgtggtactttttgtagtcgtctgcattgaaccgatcttcggcggcgatggcgtcgttgtcgttgtcaccggaccacatccaccagttccataaccaggatagcattgctttagcttgtctagcaattcctttgttatacaacgagaaaatttcgttcccttataattatagctgtacggtgcgcgtatttgtttgttaacgttacaaaaaatatccctgtccacgtccggccaatactgcaacgtgagcgcgtccaagtttgaatcttgcatatgcggaacgtacaaacgtacggcctctctcacacaatgcgcaaaactgcccggctgaatgtaatcactgtccaactttgcaggtttctcgaaagccttgtaccgatgcacgcgaacattttgagcggacgtgattttaaacttgtcggtgaattttaaccacaaatgaaatccacggttgccggtatacatgactcttgacacgttctcttccgtgtaaaacaacagaaacgccgtggcgccaatgtaaattttcagcattaaatcgtgttcgtcaacataatttttgtaatcggcgtctacgacccattccctgccgccgccgtcgtccaacggtttgacgtgcacgtcggacactttgttttgcacaatataactatacaattgtgcggaggtatcaaaatatctgtcggcgtgaatccagcgcgcgttgaccgtcatgaacgcgtacttgcggctgtcgttgtacgcaatggcgtcccacatcatgtcgacgcgcttctgcgtataattgcacactaacatgttgccctttgaacttgacctcgattgtgttaatttttggctataaaaaggtcaccctttaaaatttgttacataatcaaattaccagtacagttattcggtttgaagcaaaatgactattctctgctggcttgcactgctgtctacgcttactgctgtaaatgcggccaatatattggccgtgtttcctacgccagcttacagccaccatatagtgtacaaagtgtatattgaagcccttgccgaaaaatgtcacaacgttacggtcgtcaagcccaaactgtttgcgtattcaactaaaacttattgcggtaatatcacggaaattaatgccgacatgtctgttgagcaatacaaaaaactagtggcgaattcggcaatgtttagaaagcgcggagtggtgtccgatacagacacggtaaccgccgctaactacctaggcttgattgaaatgttcaaagaccagtttgacaatatcaacgtgcgcaatctcattgccaacaaccagacgtttgatttagtcgtcgtggaagcgtttgccgattatgcgttggtgtttggtcacttgtacgatccggcgcccgtaattcaaatcgcgcctggctacggtttggcggaaaactttgacacggtcggcgccgtggcgcggcaccccgtccaccatcctaacatttggcgcagcaatttcgacgacacggaggcaaacgtgatgacggaaatgcgtttgtataaagaatttaaaattttggccaacatgtccaacgcgttgctcaaacaacagtttggacccaacacaccgacaattgaaaaactacgcaacaaggtgcaattgcttttgctaaacctgcatcccatatttgacaacaaccgacccgtgccgcccagcgtgcagtatcttggcggaggaatccatcttgtaaagagcgcgccgttgaccaaattaagtccggtcatcaacgcgcaaatgaacaagtcaaaaagcggaacgatttacgtaagttttgggtcgagcattgacaccaaatcgtttgcaaacgagtttctttacatgttaatcaatacgttcaaaacgttggataattacaccatattatggaaaattgacgacgaagtagtaaaaaacataacgttgcccgccaacgtaatcacgcaaaattggtttaatcaacgcgccgtgctgcgtcataaaaaaatggcggcgtttattacgcaaggcggactacaatcgagcgacgaggccttggaagccgggatacccatggtgtgtctgcccatgatgggcgaccagttttaccatgcgcacaaattacagcaactcggcgtagcccgcgccttggacactgttaccgtttccagcgatcaactactagtggcgataaacgacgtgttgtttaacgcgcctacctacaaaaaacacatggccgagttatatgcgctcatcaatcatgataaagcaacgtttccgcctctagataaagccatcaaattcacagaacgcgtaattcgatatagacatgacatcagtcgtcaattgtattcattaaaaacaacagctgccaatgtaccgtattcaaattactacatgtataaatctgtgttttctattgtaatgaatcacttaacacacttttaattacgtcaataaatgttattcaccattatttacctggtttttttgagaggggctttgtgcgactgcgcacttccagcctttataaacgctcaccaaccaaagcaggtcattattgtgccaggacgttcaaaggcgaaacatcgaaatggagtctgttcaaacgcgcttatgtgccagtagcaatcaatttgctccgttcaaaaagcgccagcttgccgtgccggtcggttctgtgaacagtttgacacacaccatcacctccaccaccgtcaccagcgtgattccaaaaaattatcaagaaaaacgtcagaaaatatgccacataatatcttcgttgcgtaacacgcacttgaatttcaataagatacagtctgtacataaaaagaaactgcggcatttgcaaaatttgctaagaaaaaagaacgaaattattgccgagttggttagaaaacttgaaagtgcacagaagaagacaacgcacagaaatattagtaaaccagctcattggaaatactttggagtagtcagatgtgacaacacaattcgcacaattattggcaacgaaaagtttgtaaggagacgtttggccgagctgtgcacattgtacaacgccgagtacgtgttttgccaagcacgcgccgatggagacaaagatcgacaggcactagcgagtctgctgacggcggcgtttggttcgcgagtcatagtttatgaaaatagtcgccggttcgagtttataaatccggacgagattgctagtggtaaacgtttaataattaaacatttgcaagatgaatctcaaagtgatattaacgcctattaatttgaaaggtgaggaagagcccaattgcgttgagcgcattaccataatgccatgtattttaatagatactgagatctgtttaaatgtcagatgccgttctccttttgccaaattcaaagtattgattattgtagatggctttgatagcgcttatattcaggctaccttttgtagcattagcgatagtgtaacaattgttaacaaatctaacgaaaagcatgtaacgtttgacgggtttgtaaggccggacgatgaaggtacaacaatgccttatgtcattggaccattatattctgtcgacgctgctgtcgccgaccgtaaagtgaaggacgtggtggattcaattcaaaaccaacagacaatgttaaaagtatttattaacgaggctaatgtgtataacaaatggaatatgcttaaaggtttaatttataataataacaatgaatctgttttagtaaaataatgtagtaaaatttataaaggtagataaaaattataatattaataaaaaaaataatgttactaaatgggttcctgcgttaaattattttacgggtagacagctattaactattttatttatttttaaatttaaataaatgtattgttagaaaattgtgttgttttattagtataacgaaaaaatacatgacataaaccgcttccaattttggtcacacaaactcttgtgtggatagtttacgtaatgagttaaataggcgggcagttgtccgctaaacgtgtcggtggtcaagtagatgtgcattaatttacgacaacccaaagcggggccgcttatgtcaagtatttttttcacaaaattggtaatggtttcgttttgttccttgtacaaacacatgtcggtgtgatcgttgacgcacgagttgtacgattccgccggcaggttggcaaacaagcgcttgagatgcttgagtctgcgttcaattttataatcaaacttgttggtgaaaatgtctttcagcaagcacattaactggtcgttcaaaacgcgctgcaacgacgacaccaacacatgatattcgtttccaaaaagcgaaaaatttttgatgcagcggtccgcgttgaagggtcgtttcataatgcgcacgttgacaaaaaacacgttgaaagacagcggggctgtggttattttaacgccgttgtcggtatactcgtcgacgccgtctgcgcttgttatgtcaatttgtagcgcaaatctaaccaaatcaaactcatcgttgtactgtgtctttatgcattttatatggcggtttaagtgcaagttgatttggccgtttaatctataggctccgttttgataacatttcagcactaccaacggatccgacatgtaaacttgacgcgttagcacgtccaattcagcgtaatgttggtcgacgcatttttgtaaattagtttgcaggttgcaaaacatttttgcgcaaaagccgtaatagtcaaaatctatgcattttaatgcgcttctgtcgtcgtcaatatggcatgtcacggctgcgcctccagttaacacgaataaaccgccgttttcgcaaactacggcttcgaaacaatctttgataaatgccaactttgctttagccacaattttatcgcgcaggcgatcttcaatatcctttgtcgtaatataaggtaggacgccaagatttagttgattcaacaaacgttccataatgaatagcggcgacgcaacacgactacactgttcaaatgcgcacgcaaaacaaacccttgcaactttatttgccaatcgtaatcacagtagtttttacgagtacgccatcgcgtttgtaagcacattgctttttaaaaataatttaaatttaatgaccgcgtgcaatttgatcaactcgttgatcaactttgaactcaacatgtttggtaaaagtttattgctaaatggatttgttaatttctgcattgctaacagcgacggggttacgattcaacataaaatgttaaccaacgtgttaagttttttgttggaaaaatattattaaaaataaataaataaacttgttcagttctaattattgttttattttttataaaataatacaattttatttatacattaatactttggtatttattaatacaattatttacaatactttatttacactataatactttatttacattagtactaaattaatactaaattacgctaatactaaattaatactttatataatcaaaaataatactttatataatactttctaatcatcataaacgggtaatagttttttctcttgaaatttacgctgcaactcttcgctaaaacacatgggcggtggagtgggagcgggtggagtaggagtccttacgggtttgatgggcgacagttctctggacttgcggaacagcttgggcgaaagcgtcggcgtgcgccgactaatgatttcttcatccggcaacggaggctcgcacattgtgcacgcgtccggtgaggtacacaaaactttcttgggcacgctgtacaccggcttgggcacgctatatgtgttgccaaaatagaactcgttgtggttgccgaacggagacgatgggtgtgaagacggcgatggctgtgaagacaagtccgaaggcgcgataaaagatgaaagtgtttctgaaaccgaagtggtggtagaagtggtagaaggcgggtgcgttacggcaaccacgctgctgctatttctgccttcggagaccacttccagcaatctagagttactctctcgttcttcgcggcgatagtcaatgtcgcaataatgttcataagatgccttttcggcttcggcgcgccttttcatgtatatgttgtgacgcatctcctttaactgcacgtacaaattccagcattgcacagccagtatcgtaagcacgcccattatgattacgggataattttgattaaacacggtcggctcgtgatcgcttacaatcgctcggcacatgatgcattttttgtaaatgttcacatacacacagttttggctcaaggtttcggtatttgcgtagtcaatttccagatacacgatagagttccagcacattgattccaaatcgtagtgacgatataaaacatctagcgccggtagatgaccatttttgaacacgtagatttgaaacgcggcaaacagcatccaacacagcccagtgatcacgtttaccataatacacgtgatagcgacgtaaaagttttctttcgcattgaaatttacatttgtgtttgaagagctgctgcgatttttcgtccacacgataatcttccatataaaataaaacatgtaaaataatatccacatgccgaacgccagcattatcggtatagatagattgataaccgattgctttccttcaatttccagcaaaaacgcgtatctgctgtctatcactcccattatagataacacaaacactatcagatatgctaataataatgaggcattaagcccgaattgtaaaactgcagtgattttatttaacattttgaatatttaattcaacaactaagtaatggcaatatgtatcgagtactgatcgtgtttttcctgttcgtgtttctttatatagtgtaccagcccttttatcaggcatacttgcatatcggacatgcccaacaagattacaatgacacgttggacgataggatggattacattgaatccgtaatgcgtagaaggcactacgtgccgattgaagcgttgcccgcaatcaggtttgatactaatctcggcacgttggccggtgacacgattaaatgcatgtcggtgcctttgtttgttagtgacattgacctgccgatgtttgattgtagtcagatatgcgataacccgtctgcggcgtatttctttgtcaacgaaacggatgtgtttgtggtcaacggccacagactgacggtgggcggatactgctccactaatagtttgccccgcaactgtaatcgcgagacgagcgtcattttaatgagtctcaatcagtggacgtgcatagccgaggacccgcgttactatgcgggcacagataacatgacgcaactcgcaggcagacaacactttgaccgcattatgcccggacagagtgataggaacgtcctgtttgaccgattactaggccgagaggtgaacgtgaccactaacacgtttcgccgcagctgggacgagttgctggaggacggcactaggcggttcgaaatgcgctgcaacgcccgagataacaacaataatctcatgtttgttaatccgcttaatcccctcgagtgtctcccgaacgtgtgcactaacgttagcaacgtgcacaccagtgttagacccgtatttgaaacgggagagtgtgactgcggcgacgaagcggtcacgcgtgttacgcacattgtgccgggggacaggacctctatgtgtgccagcattatagatggcctggataaaagtacggcatcatatagatatcgcgtagagtgcgttaatctgtacacctctattctaaattattctaataacaaattgttatgtcccagtgacacttttgatagtaacacggacgcagcttttgcctttgaagtgcccggctcctaccctttatcgcgcaacggcatcaacgagccaacttatcgcttttatcttgataccagatctcgagttaattacaatgacgtcagagggcagttatcttaattgtgataacacaaacaataagtcatttaaatgttacgtcagtagttagtatataagccgtacatgttggcttgcaaattcagtcaatatcaggcttttatcatggacggtgtaaagctgctagggacgtgcgcgctaataattttgttatcgacgacgagtacagttgtcgggcgtgaccgtatcacgtttacgccgatagaagatagcgcaggcctcatgtttgaacgcatgtacggcttgcgacatcatacagacgacagatttgtgtttgtgaaaaaattcaattttgtttcggtgctgcaagagctcaataatatcaaatctaaaattgaattatatgaagcgcaagtttcaacttgcacaaacgtcagacaaataaaacagaacagatcgagtatcatcaaagctcgcattgaaaatcagctgcagtttttgacgcaactaaacaaaaatctcatcacatactctgtggaaagcagcattttaagcaacgacgtgctggacaacatcgatctggaatatgacgacagcggtgagtttgacgtttacgacgaatacgaacagccttcgcattggagcaacatgactgtatccgacgcgcaagctttgctccgaaacccgcccaaagacagagtaatgtttttggacatggttaccaccagcgacgtgagcagcaaatacgaagaatacataaactgcattgtgagcaaccgtaccgttgaaaacgagtgcatgtttttagccaacatgatgaacgtgctcaacgacaaattggacgacgcagcagctttggccaagatgctggagcgaatagtaaaacaaacgcgaaagaacaaactcaacatctccaacacggttatagacgacgacacgctgctaacggaaatgaaaaaattaacacaaactttatacaaccaaaaccgcgtgtgggtagtggattttaacaaggacatgaatagttatttcgatttgtcgcaagcgtataaattgcatttatatgttgatttaaacacggtcattatgtttattaccatgccattgttaaaatccaccgccgtttcgtttaatttgtatcgcgtcatgacggtgcctttttgcaggggcaaaatgtgtctgcttatcatttcgggcaatgaatactttgggattacagacagcaaaaactattatgtgcccgtatctgataactttagacaagattgccaagagtttacgggctacaatgagtttttgtgtcccgaaactgagccgattgccactatgaactcgaaagtgtgcgagattgaaatgtttatgggtcgatatagcgacgacgtggacaacatgtgcgacattagggtggccaattataatcccaaaaaagcttacgtgaacactttaatagactaccgaaaatggttgtacatttttccaaacacgaccgtgtccgtccactattattgtcacgacgcgcttgtagaagttgatacaaaagtttcgcccggcgttggtgttatgttttcgactatggcgcaaacgtgttcgattagaataacgtatgatgtgaccataactgtagattcgcgattttatgtcagccattcaactacatactggcctaaaaagaaatttaattttaacaactacatcgaccaaatgttgcttgaaaaagcgaccaccagttttataccgactgttgacaattttacccggcccgttttattgcaacttcctcataaatttcacattaaagattacacatcgacgccccatcattttttccatcagtctaaaatttacaccaacagcgcggcgcccgacgaagactcgcaagacgacagtaataccaccgtggtaattatcgctattgtcgctgcaatgatcctattctgtggattattgttatttttgttttgctgtataaaaaaacggtgtcatcaatcaaataacgtggttgtgcaatacaaaaataacaatgaatttgtcacaatttgcaataatttagaagacaatcgagcatacattaatttacctaatgaatacgatagcgatgatatgccaaaaccattgtaccctttacttggctttaatgatgatttgttaaaagatgataaacctgtgttgtaccctatgattatagaaagaataaaataaaacatgtataattgaaataaatatattatttaataaaatgttttttatttatatactattttctattacatattccaatgcacacaaatgtttaatggctatcagttttaattttactaattcgtctaaacaaaaattattcacttgctgtttttcatccatttgacatatggcgtttataaataattcgctgtgttttatgaacgaatcgtaaaccgctgcctgggccttcagcacggtcggcgcattgtatttttgggtaaagtacgcaatatttttagtcaaacacagagattttaaatctttttcatttatatccaagtcggaacaatcgtatacaaaatctagcttttcactttcgggcgcgcccagatactggtttacgagttcgagctgctccacttggcctttgatatcggccgctatgcacaacattttgtcgattgcagtttcattgtttttaacataataatttttaacttttttattttgcaatttaatcaaactatttaaattcgcttgacctttcttacaaagcgcagttaatatgcaagacattttgacttataataaaaaacaaaacttttatatattcatttattgttcaataataacaaatattccaggcttaaaagctaacgaatagggcttttcggtaattttcttattattcatgtccgtcatctgcatctctttgccgtacttgacgccgtcaatggtgcccatcatgtacattttaatctcctccgaaggtccgtctattttgtccatttcgaacaatctatcaaaatcttcaacgctcattctctgcatatcaagaggaacgtttctgatctttccggtggcgtaaattgatccgttgttgtcacggttgattatgtaaaaccgacgaatcaacatgtcgcgctcgctagttttgttcttatccggcaaatgaatgcacacgtttggttccatcttcaaaggaaaatcgctttgcaagtgtttttgcaaaatgttgccaaatatattgttgtgtttgtgaatgtctccgtattgaatgctaaaaaactggccaaagttgcttttggcacgttttatggttccaaagtcggaaaaccaaaatccgcagggcttgccctgcactcttggaccgatggtgtacgtagtcttgccgttggccggctccaacaccacgatatttttatcgggctcgggatacaacttgtcttcccattcgtgcaaactgttcaaattagacagtcgacaaaattcgtttttcaaaaatctgccttcgaaacaactacaattcagtattgaaaagttgcctcgtttcacattaatcgccatctgctcctgccacaacatcttcgtcaactcgtgtggctccaattgaatggacgacggcgtaaaatagcacattacgcccgtttcgtcgtgtttcacgttaaaagcgccgctgttgtacggcaccagctgctggtcctcaccaccttccgatctttcccgcttcggctggttgtcgtcgctgctcgaatatccatcgccaatcttgcgtttagttgccatgctaccgacgtgcgctgtctgctgtggttcaagtctaattgaagtgtttcacagaatataagatatataataaatatggacgactctgttgccagcatgtgcgtagacaacgcgtttgcgtacactactgacgatttattgaaaaatattccttttagtcattccaaatgcgcccctttcaagctacaaaattacaccgttttgaagcggttgagcaacgggtttatcgacaagtatgtggacgtgtgctctatcagcgagttgcaaaagtttaattttaagatagatcggctaaccaactacatatcaaacattttcgagtacgagtttgtagttttagaacacgatttgtccacagtgcacgtcattaacgccgaaacaaaaaccaaactgggccatataaacgtgtcgctaaaccaaaacgacgcaaacgtgctcattttgaccgtaactttaacgagctaaaatgaacgaggacacgcccccgttttattttatcagcgtgtgtgacaactttcgcgacaacaccgccgaacacgtattcgacatgttaatagaaagacatagttcgtttgaaaattatcccattgaaaacacggcgtttattaacagcttgatcgttaacgggtttaaatacaatcaagttgacgatcacgttgtgtgcgagtattgcgaagcagaaataaaaaattggtccgaagacgagtgtattgaatatgcacacgtaaccttgtcgccgtattgcgcgtatgctaacaagatcgccgagcgtgaatcgtttggcgacaacattaccatcaacgctgtactagtgaaagaaggcaaacccaagtgtgtgtacagatgcatgtccaatttacagtcgcgtatggatacgtttgttaacttttggcctgccgcattgcgtgacatgattacaaacattgcggaagcgggacttttttacacgggtcgcggagacgaaactgtgtgtttcttttgcgactgttgcgtacgtgattggcatactaatgaagacacctggcagcgacacgccgccgaaaacccgcaatgttattttgtattgtcggtgaaaggtaaagaattttgtcaaaactcaattactgtcactcacgttgataaacgtgacgacgacaatttaaacgaaaacgccgacgacattgaggaaaaatatgaatgcaaagtctgtctcgaacgccaacgcgacgccgtgcttatgccgtgtcggcatttttgcgtttgcgttcagtgttattttggattagatcaaaagtgtccgacgtgtcgtcaggacgtcaccgattttataaaaatatttgtggtgtaataaaatggtgttcaacgtgtactacaacggctattatgtggaaaaaaaattctccaaggagtttttaattcatattgcgcctgatttgaaaaacagcgtcgactggaacggcagcacgcgcaaacagctgcgcgttctagacaagcgcgcctacaggcaggtgttgcactgcaacggcagatactactggcccgatggcacaaagtttgtctctcatccgtacaacaaatctattcgcacgcacagcgcaacagtcaaacggaccgacagctcgcatcgattaaaaagccacgtggtcgacaaacgaccgcgccgctctttagattctcctcgcttggacggatatgttttggcatcgtcgcccataccacacagcgactggaatgaagaactaaagctgtacgcccagagccacggctacgacgactacgacgacaatttagaagatggcgaaatcgacgaacgtgactctttaaaaagtttaaataatcatctagacgacttgaatgtattagaaaaacaataaaacatgtattaaaaataataataataaaactatattttgtaatatataatgtattttatttaaaaattgtctattccgtagttgagaaagttttgtcttgacttcataactctcttctccatattctgcagctcgtttacgttttttgtgacgcttttaattttctcaaaatgctggctgtcaatagttattttttgcttttgtctattaatttcttccaattgagattttaaatctcgctgagattgagatgcgttgtaattccttgagaacatcttgagaaaacatacagatgaggtaaaacagcatcttttatccaaattaggagttaattattattcatttgtatcgcgaccatttgctcgtacacatcttccataaaatggttatttttattgcgataagtgttggcattgacattttgcaaatgtcgtaggttaaaggggcaaatgggctgcgtggccgataaaagattccagttcaacaatccctcttcgcccccgtttaacttgaaaatggcgctacacgtttctacgctatcgtgttcctgttgagtggcgcacggttcgaccagtatcatcttgtgatatgcggttttgacattcatgtgcaacggaataacttgcgggtcatcgcattcgtcggaattaagctttaaatggcgtccgtatgctttccaaagtttttcgtcgtcgaaccgcggcactgcttgcaagtcgacgcggggaaacggcgctctgtacaaaacgcctaaattcaaaaactgattgcattgttgcagctctgtccaatcgacgcgatttttgtaattttgaaacagcatcaggttgaacgccgcgctggcgcgcacgtttgtaatcactgtgtaattgatcagcttgtgccaatactgggcattgaaattttcttcaaactcatttctaaactctggatgcgcaaacatgtgtctaatgtagtacgcgggcggggcgttgaacgcagtccatttgtcaatacacttccagtctgaatgtaacgtgttcaccaaaccgggatattcgtcaaacacgagcatgtgatccgaccacggtatgctgtgggcgatcaattttagttcttgcacgcggccttcgcgtaagcaatacaaaatgagcgcgtcgctgatcttgacacagtcttgcatgtacgcggacaaattaacgttttccatacagctcacattgtttattagcgccgtgttcaagtgtttgtatttggacacataatcgtagttgatgtactgtttaatgggttcttgaaaccattcttttagtagtatgtgactggccactatgcgtttccaatttaatttgtgtgcgtatttttgctgcaccgacaacgagaggttattgtaatttttggatatttcttccatgtccaacaagtccccaaacgcgagtataaaatcttgcgtcaaaaatttttgctcagacaccaacgaccagatcaaatgtgatttaaacctgttggcgattgttatcgacaacggcgaaattgaaataattttccaatccaacttgttgcgaaacacgtgaataaaatcgacgcgtccgtaacattcgcgcgatatgcgcttccaaaacgtgtcatcttgcaaattaagcaaatagacacgattgttgggagatttgacggccaattcaattatttttatatattctttttgctttaaagcgcgttgtagcacttgggttggagccatgtcgactgaagctccacgctgtttgaagcaaggtgaccgttttggtcggcatgttcaaacgtcgattacatgtttgctttgcatcaaaatggcgtaattaattaagaaacaacatgaaagccatctgcatcattagcggcgatgttcatggaaaaatttattttcaacaagaatcagcgaatcaaccgcttaaaattagcggctatttgttaaatttgcctcgaggtttgcacggctttcacgtgcacgaatatggcgacacgagcaacggttgcacgtcggccggtgagcactttaatcccaccaatgaggaccacggcgctcccgatgctgaaattaggcatgttggcgacttgggcaacataaaatcggctggctacaattcactgaccgaagtaaacatgatggacaacgttatgtctctatatggcccgcataatattatcggaagaagtttggtcgtgcacacggacaaagacgatttgggccttaccgatcatccgttgagcaaaacaaccggcaattctggcggccgtttgggatgcggaataattgccatatgtaaatgatgtcatcgttctaactcgctttacgagtagaattctacgtgtaaaacataatcaagagatgatgtcatttgtttttcaaaactgaactcaagaaatgatgtcatttgtttttcaaaactgaactggctttacgagtagaattctacttgtaacgcatgatcaagggatgatgtcatttgtttttcaaaaccgaactcgctttacgagtagaattctacttgtaaaacataatcgaaagatgatgtcatttgttttttaaaattgaactggctttacgagtagaattctacttgtaaaacacaatcgagagatgatgtcatattttgcacacggctctaattaaactcgctttacgagtaaaattctacttgtaacgcatgatcaagggatgatgtattggatgagtcatttgtttttcaaaactaaactcgctttacgagtagaattctacttgtaacgcacgatcaagggatgatgtcatttatttgtgcaaagctgatgtcatcttttgcacacgattataaacactaatcaaataatgactcatttgttttcaaaactgaactcgctttacgagtagaattctacttgtaaaacacaatcaagggatgatgtcattatacaatgatgtcatttgtttttcaaaactaaactcgctttacgagtagaattctacgtgtaaaacacaatcaagggatgatgtcatttactaaaataaaataattatttaaataaaaatgtttttattgtaaaatacacattgattacacgtgacatttacgatggcgaacaataatttcactttttatattaggacacgacgtgtatataggaaagcttaagcgtttcaataaagccatggcgtacacgctaagcttgcccagcttgcggctctttgaaatctgtagttttcggggagtaccgtcgttcttcagtgccacatacgtcaacttgcgatcgtacactttataatacgtgttgtagttatttttttccagaaattccctcataaagcaatccttggataaagtttttgatccgtacagttggccacaccggtccatgcacaggtacacacacgtgatggcgttttgaatgacgatgcgatttctgtcaacggcaacgcgcttgaatatggtgtcgacgttgtccgattcaatggttccgtaaacagctccgtctggatttactgccaaaaactgccggttaataaacagctggccgggaatagacgtgcccgtgatgtgtgtcagcagagctgagcagtcagccatagaggctagagctacaagtgccagcaagcgatacatgatgaactttaattccccacagcaaactggcgcttttatataaaaatttgggccatttttggcgattagataatttttgaagattagataatattgagattagttaataatttgtgtgattagataactttttagggtattgcgcattataaatcaaggtcgagttgtataaactgctctggcgtgtaaaactgcagacttaagttttttgcaaacactcggtctgaatcgctaaaatctttctgaccggtggttagattaattcggccagccgcgtcgcccacataaaaagattgttccttgtcaatatgcgtaaactgtttggccatctcgcgccacattcccgtgtcgggctttcgatgctcatccttgttgggcgacacataaaacgatatgggcacgccagtagcttttttaatattctctaatttatataataaatcgctcgctttgattttgccggaacctaaatgggcttggttcgtaaaaacaactaaatcgtagcctaattcgtacaaacgctttagcttgtgtgcgcacggaaggagctgccagtcgtctgggttttttggaaatttggaccgtgtctttgagctaattagcgtgccgtccaaatcaaaagccgcaattttggttcttttagcgccgtcatgaaccgcgtacgcatacaaatcgggctgctgtaacgtccacatggtgaatgcatcttactcaaagtccatcaattcgtacgcgtttgtgtccaggtcgggcgttgaaaaattgtagcttgccattagatcggatagcgattcaaattttgtaagcgtttgtagcgcacgtttggcatcttgtttaaaattacacgacgacagacagtaaaaatattcctcgataagcatgactacacccatatcactgtttaagtgctcgacgtagttgttgcatgttatgtcgcgtgtgccgcgatacgcgtgatttcggtgaaaatcacaccacaaccagtcggcgtgcgtgtaacaaagtcgacagcgaaacaatttatcgttttccaaaaaatttaaatactcgacagttttgcagcttagattccgcgtttgattcaccttaaaatcgtcgtcagcctctataatctcgggcaacagcttgccttgttgccccatcgtatcgatcacctcccccaagtggcccggtgttatattaagtcgtttaaaatcatttattgcttcctgcacgtcggcctggtaatttttgaccacgggcgtggaaatcaattgccgttgaagggaaataattcgtggtgtgggtatcggccgcctgttgcacaattccaccagcggtggaggcaagggcgcattcacagcaaccgttgtcatttataagtaatagtgtaaaaatgcaaatattcatcaaaacattgacgggcaaaaccattaccgccgaaacggaacccgcagagacggtggccgatcttaagcaaaaaattgccgataaagaaggtgtgcccgtagatcaacaaagacttatctttgcgggcaaacaactggaagattccaaaactatggccgattacaatattcagaaggaatctactcttcacatggtgttacgattacgaggagggtattaataataacaataataaaaaccattaaatatacataaaagttttttatttaatctgacatatttgtatcttgtgtattatcgctaaccattaaaagtgctggagccacagtgttgcggcgagtctttatagaagatcgttgtttggctggaactgagcttttccttttcctgctgccgctaatgggagtgggcacgtactctgtagtagacggtgcaacgggcaacttgagcgctaccgtcttaaatttggccatacttttagtgatgaaatcgcgcgttaacacttcgtcgtaaatgttacttagcagaggcgcaacattgtgattaaatgtctcgtttaacaagctgtaaaactccgaataaagcttatcgcgcatttcgcagctctccttcaattctgccaaatttgcgttggtaagcaccacagtctgtctttttttgctcgctggaattgctgcgttctcgcttgaagacgacgatgtcgatcggtcggccatttttttgcccagcttttcagtgtgatcaaaaatgaacacaaaatctgccaattcgggcttgtttttcaccaaatcccacatggccgggctactaggccactcgggctgcttgatcttagtgtaccaactgttaaacaaaatgtatttattgttgttaatcactttcttcttgcgtttggacattttgcgttcgtcttgcatgacaggcaccacgttaaggatatagttaatgttctttctttccaagaaatttacaataacggccagctggtccatgttggatttgttgtaagagctcgattccagtttattcaacagcttttcatttttgcacacggccgcagtctccggagattgttgctccggcacgtttaccatgtttgcttcttgtaaacctttgaaacaacccgtttgtattcttgatgatatatttttttaatgcccaacaacctggcaattcgtttgtgatgaagacacaccttacgcttcgaacatttgtcggtgattactgtgaaatggcctaaattagctcttatatattcttttatacgctcaaacgacacgatgtccaacatgtgcgcgcagacgttttctgtgttcatcgtgtgcttgagcgtgttgatggcttccctgaacagcgcttgtatttcgctgcgagtcaagcagtccgaatcacacccgcctaagtgcgtgcaatttttggggggcatcgttgtctatctttttcagagtggcgtagaaaaagtcctgcaattgcctattatcaaaacgcgccttgacgctgcgcacaaaatcaaaaaattcaatgtaattgctgtaatcgtacgtgatcagttgtttgtcgttcatataattaaagtatttgttgagcggcacgatggccaggctgcgcgctatttcgcaattgaagcgtcgcggttttaacattatacggtagtcattgccaaacgtgcccggcaacaacttcacggtgtacgtgttgggtttggcgttcacgttaatcaagttgccgcgcacgacgcctacgtatatcaaatacttgtaggtgacgccgtcatctttccattgtaacgtaaatggcaacttgtagatgaacgcgctgtcaaaaaaccggccagtttcttccacaaactcgcgcacggctgtctcgtaaacttttgcgtcgcaacaatcgcgatgacctcgtggtatggaaattttttctaaaaaagtgtcgttcatgtcggcggcgggcgcgttcgcgctccggtacgcgcgacgggcacacagcaggacagccttgtccggctcgattatcataaacaatcctgcagcgtttcgcattttacatatttgacacttaaaaaattgcgcacacgagcaccatcgtttgatacctaattgcaactatttacaatttatcagtttacgttgaacccgttttaattttttagatccgtccttgttcagttgcaagttgactaaatgacaaaatttttcggttctgcaaaaccgcccttgtctgttccacccgttgtatttgaaaaaactttttttcacgcggcgacaactgcttgtataatattgcccaatgtaaacatgcaaaattttgttactctcgtcaaaacagcggttggcgttccattccataatttttttattatttatcaacgatggccattgtaaattgtcgtcatttatacgcatcatatgatttaacaaaagcttttcgtatagcggaacttcaattcccttggaacatttttcaaacgataatttaatttgtttctcggttggcagcatttcatgcttgattaacaatcgcctgacttttatagccacgtttatgtctttgcacagcaaatgtgggttgtcgacaatgtaatagtgcaaagcatttgttacggcaaatgcgtagtttgatttgacgacgccctttttcttgacgggcattgcggcttttaaaattacttgcaagcattgtacgaatacctctttgtgtttaaacaataatatggacaaacatcggcgaaacaatttgtaataattatgaaatcccaaattgcaggttttaaacttctttgttacttgttttataataaataaaatttgctgacccatgtctgcgcccacaactttaattaaccatttgtgcgcatattgattgtctcgttgttcccaaccggaaaattgattgatctcgagccaccggcattggtcgtttgataccgtcgttaacgccgacgctcctgcctgtttgattacgggttctaaaagacgaaacagcagcgtaaatttgtttttgcgtcggtagtattttggcaggcaataatcaaaaaaatccgtaagcaattctctgcatctattaatattcgttgcgtacgaatcgagtttttcaaaaattactttgtttgtatgaaaataacgtttgggcttctcacaataataatcttcgttgtagaacagaaacggtttgcgagaattggcacgtttgtccatgattggctcagtgtaacgattgattcaaatcaaaattgacaacacgtttgccgtaatgtgcaccggttcgcacacgtttgccgcgtatgtaatccatgtttatttcgctgtcgcaattgattacacgattgtgttgggcggcgcgttttattgaatttaggcgacgcgtcgacaactccaaaggattgtaaagcgcagatttttccagagtaaacgagtttaagtggccaccgttgaaccattccagagccacgattgtgtacagcaaaaagaatatttctttgtcgacgttttcaaacgcaaacttgttttttaggcaatagtagtaaaattttaacgaattgtataaataaaacataaaattgccatttttaaagtaaaattctacatccgtgacgaacaaaaggtttactattttgttctccaacaagtgtgccaattttcttaagtacaccattgaatttttgtcgtcgtccatctcgatcaacaacacgtacggcgttttggaatttaaaattattctaaaattttcctgttgcaacgattccacagcgtccgaccaatatgacgctgccacctctagacagatgtatttcttggaaaacacgtgtcgtttgataacctcgctgatggacgtgatcgattgtaaatacttttcaaacgtcgcgtcttcccaaccacgcaccgacacgggcgctgtcgtgtcgggctgatgtttgaaatccaaaccactctgaattaacttggttgtgattcgtatgctcaactgttgacccaacgtgtagtgatcttcgtaggcgcgctcccacatcacgttacacacaaatttgacgagatcatcaacgtctttctgttgcaaaattcgccgcaaacgcgccacatcgcccttgtaccaccgatctcggcacacaagctgtagcatttttaaatcgtgatcgctcaagctattaattctggttagatttatatagtcgtcaatatcctcgggcgtggtttgcgtcatgtctgtaaaacgtgcaaaatcaaacatttttatgttgtagtcgaatctaacaaatccatcggcgttcacttgcacttcgcgctttacaaaacgaggtagcgtgtaatcgaacccgtttaaatagattgcgtacaaaaccagcacttcatcttccagtttgcacgcttgcggcaaaaattgtgtggtgtgctccaaccgggtgacaaacatgactatggaaaataacgcggaattcaacagacgactagagtacgtgggcacgatcgccacaatgatgaaacgaacattgaacgttttacgacagcagggctattgcacgcaacaggatgcggattctttgtgcgtgtcagacgacacggcggcctggttatgcggccgtttgccgacctgcaattttgtatcgttccgcgtgcacatcgaccagtttgagcatccaaatccggcgttggaatattttaaatttgaagaaagtctggcgcaacgccaacacgtgggcccgcgttacacgtacatgaattacacgctttttaaaaacgtcgtggccctcaaattggtcgtgtacacgcgcacgctacaagctaacatgtacgcggacgggttgccgtattttgtgcaaaatttttcagaaacaagctacaaacatgttcgtgtgtatgttagaaaacttggtgcgatacaagtagcgacattatcagtttacgaacaaattattgaagatacaataaatgaactcgtcgtcaatcacgttgattagataatgtccgtgttaaatgtgatatcttagattacgagcgcgcaataaccatagtttaatcgaagagaatagccgtcgccacaatggataattacaaattgcaattgcaagaattttttgaccaagcgcccgacaacgacgatcccaactttgaacatcaaacgcccaatctattggcgcatcagaaaaaaggcatacagtggatgattaacagagaaaaaaacggccggcccaacggcggcgtgcttgccgacgacatgggactcggcaaaacgctctctgtgctaatgttaatcgcaaaaaacaactctctacaattgaaaactctaatagtgtgtcctttgtctttaatcaatcattgggtaaccgaaaacaagaagcataatttaaattttaacattttaaagtattacaaatctttggatgccgacacgtttgagcattaccacattgtggtgaccacgtacgacgttttattggcacatttcaaattgatcaaacaaaataaacagtcaagtctgttttcaacccgctggcatcgagttgttctagatgaagcgcatattatcaaaaactgcaagacgggcgtgcacaacgccgcgtgcgctttgaccgcaacaaaccgatggtgcattaccggcacaccgatccacaacaagcattgggacatgtactcgatgattaattttttgcaatgtcgtccttttaacaatccaagagtgtggaaaatgttaaataaaaacaacgactctacaaatcgcataaaaagtattattaaaaaaattgttttaaaacgcgacaaatctgaaatttcttctaacattcctaaacacacggttgagtatgtacatgttaattttaatgaagaagaaaaaacgttgtacgataaattaaagtgtgaatcggaagaggcgtatgtgaaggctgtggcagcgcgtgaaaacgaaaacgcactaagccgattgcagcaaatgcagcacgtgttatggctaatactgaaattgaggcaaatctgctgccacccgtatttggccatgcacggtaaaaatattttggaaacaaacgactgttttaaaatggattatatgagcagcaagtgcaaacgagtgctcgacttggtagacgacattttgaacacaagcaacgacaagataatattggtttcgcaatgggtggaatatttaaaaatatttgaaaacttttttaaacaaaaaaacattgctacgttaatgtacacgggccaattaaaagtggaagacaggattttggccgagacgacattcaatgatgctgccaatactcaacatcgaattttgctgctttccattaagtgcggcggcgtcgggttaaacttaataggcggaaaccacattgtaatgttggagcctcattggaacccgcaaattgaattgcaggcgcaagaccgaatcagtcgtatgggacaaacaaaaaacacgtacgtgtacaagatgctaaatgtggaagacaacagcatcgaaaaatacattaaacaacgccaagacaaaaagattgcgtttgtcaacacggtctttgaagagactctgctcaattacgaagacattaaaaaatttttcaacttgtagctggtaagtcgtcatgaacacccgatatgctacttgctatgtttgcgacgagttggtgtacttgtttaagaaaacgtttagtaacatgtccccttcggccgctgcgttttaccaacggcgcatggccattgttaaaaacggtatcgtgctgtgcccacgttgttcgtcggaactaaaaattggcaacggcgtttcgattccaatttacccccaccgcgctcaacaacatgcacgacggtcgcgttaagacgcaagcgcttcgagttttggcccgctcgctacctccgctgtacgactcgaccgtcgatcgacacggctgcaaggtgttcacggtgcggcgctacaacagacgcgtaatcgactttgcgggcattcgcaacaaaacgctggaaatcattaaaacggatagaaacttgccgctcaacacagaatgcaatgtgaaagttgtcgacagtgcatgcatgcgttgcagaaaaagtttcgcagtttaccccgccgttacctatctgcattgcggacattcgtgtctgtgcaccgactgcgacgaaacggtaaacgtggacaacacgtgtcctaaatgtaaaagcggcattagatataaattaaaatacaaaactttgtaacatgttgccctacgaaatggtgattgccgtgttggtttacttgtcgccggcgcagattctaaatttaaaccttccttttgcataccaaaaaagtgtgctgtttgccagcaactctgcaaaagttaacgaacgcatcaggcggcgagcgcgtgacgacaacgacgacgacccctatttttactacaaacagttcataaagattaattttttaactaaaaaaataataaatgtttataataaaactgaaaagtgtattagagcgacgtttgatggtcggtatgtggttacacgcgacgttttaatgtgctttgtaaacaagagttatatgaagcaattgctgcgcgaggttgacactcgcattacactacagcaacttgttaaaatgtatagtccagaatttggtttttatgtaaatagcaaaattatgtttgtgttaactgaatcggtgttggcgtctatttgtttaaaacactcgttcggcaaatgcgagtggttggacaaaaatataaaaactgtgtgtttacaattaagaaaaatttgtattaataataagcaacattcgacatgtctatcgtattgattattgtcatagttgtaatatttttaatatgttttttgtacctatcaaatagcaataataaaaatgatgccaataaaaacaatgcttttattgatctcaatcccttgccgctcaatgctacaaccgctactactaccactgccgttgctaccaccactaccaacaacaacaacagcatagtggcctttcggcaaaacaacattcaagaactacaaaactttgaacgatggttcaaaaataatctctcatattcgtttagccaaaaagctgaaaaggtggtaaatcccaatagaaattggaacgacaacacggtatttgacaatttgagtccgtggacaagcgttccggactttggtaccgtgtgccacacgctcatagggtattgcgtacgctacaacaacaccagcgacacgttataccagaaccctgaattggcttacaatctcattaacgggctgcgcatcatttgcagcaaactgcccgatccgccgccgcaccaacaagcgccctggggcccggtcgccgattggtaccatttcacaatcacaatgcccgaggtgtttatgaacattaccattgtgctaaacgaaacgcagcattacgacgaagctgcgtccctcacgcgttactggctcggcttgtatctgcccacggccgtcaactcgatgggctggcaccggacggcaggcaactcaatgcgcatgggtgtgccctacacgtacagtcaaatgttgcgcggatattcattggcgcaaattaggcaagagcagggaatacaagaaatcctaaacacgatcgcgtttccgtacgtgactcaaggcaacggcttgcacgtcgattcgatatacatcgatcacattgacgtgcgcgcttacggctatttgataaattcatactttacgtttgcctattacacgtactattttggagacgaggtaatcaacacggtgggtttgacgagagccatcgaaaacgtgggcagtcccgagggagttgtggtgccaggcgtcatgtctcgaaacggcacgttgtactccaacgtgataggcaactttattacgtatccgttggccgtccattcggccgattactccaaagtgttgaccaaactttcaaaaacatattacggttcggttgtgggcgtaacgaataggttggcttactacgaatccgatcccacaaacaacattcaagcgcccctgtggaccatggcgcggcgcatttggaatcggcgcggcagaattatcaactataatgccaacacggtgtcgtttgagtcgggtattattttgcaaagtttgaacggaatcatgcgcatcccgtcgggcaccacgtccacgcagtcgttcagaccgaccattggccaaacggctatagccaaaaccgacacggccggcgccattttggtgtacgccaagtttgcggaaatgaacaatttgcaatttaaatcgtgcacgttgttctacgatcacggcatgttccagctatattacaacattggcgtggaaccaaactcgctcaacaacacaaacgggcgggtgattgtgctaagcagagacacgtcggtcaacaccaacgatttgtcatttgaagcgcaaagaattaacaacaacaactcgtcggaaggcaccacgttcaacggtgtggtctgtcatcgcgttcctatcacaaacatcaacgtgccttctctgaccgttcgaagtcccaattctagcgtcgaactagtcgagcagataattagttttcaaacaatgtacacggccacggcttcggcctgttacaaattaaacgtcgaaggtcattcggattccctgagagcttttagagttaattccgacgaaaacatttatgtaaacgtgggcaacggcgttaaagccctgtttaattatccctgggtaatggtcaaagaaaataacaaagtgtctttcatgtcggctaacgaagacactactataccatttagcgttataatgaattccttcacctctatcggcgaaccagctttgcaatactctccatcaaattgctttgtgtatggaaacggtttcaaattgaacaacagcacgtttgatttacaatttatttttgaaattgtgtaattatatttagggagaatgtgatattcaaaagactgactgttaacacaaaagactgatattgttgttgttacaaaatagataataaaacaaaaaataaattaaatattatttatttattaaactgtttaattttaatgctaacgcgtacaaatcacgctgttccgacgtggacatggaattgcgcagaaaagtcttgatagtgtcgatttcttcgccgtcatccacttccatatatttgatttcttcctcgatttgcatttccaagtttgcgtattcttgcaaataataatctagtcgttgggcgacctcgccaattttaaataatacattatccgacaccaaatgccagcgagtgactgtgcgctccatcatcctggcactttttaatgtgaatattaaaaggttgttgcatatatatcgttaaacgtttatgtttactttcacgttagctcgtttcattgatgtaaacatttagttttataacagcgtcggtaattttattttttaaagtaaacagaccaaaatcaaaggtgtcttcgacaggtacgattattttcccattgacactgttttcgtgcacagatataattttatcaccgtttattattttgcccaaacacacgtactcgtttcttctcaagccaactatttctaaacaattcacttttctattatcgtgtacgcaattaaaagtaaacgaagcgctacaattgtcgtattctattacaattctgcggcatttataaaatttattaatgttgacgcaaattccatgcagcgcatccatttcgtactgcaaatgcggcgcaattaaaaaatttcctcgtcgttgttaacaatcttgggcgctaaaaagcacgccaacacgcccacgtctttaatgcaatattccaatttgaacggcagttcctcggacatgtatattgtcacggtgggcgccaaaggagcggctttagcaaaatgacacaagtaatcgcccgcaaaagtgtgcgttacggtttgctttgctttgagaacggaaaagttttcgttgtccgcgctcatctgcacgtccgccgagccaatgtcgccatttgctctaaactgcagacccttcttggaacacgacacaataatatcgtggtcgaattgcgtcatgtctttgcacacctgcgcaaactcgacgctcgacatgtggacgacgcaatcgtaatcgctatccggaattcccaaatgttccacgtcgatgcacatcaacttgagcgtgtacgtgcagattctattgtcgttgttgaacacgaacgccatcacatcgccctgatcttccgctttcatcagtacagagctgcgctcgttaacgcatttgacaattttacttaaactgtttatggacacgttgagcggcacgttgcggtcacatctatattttttgaaaccctcggcgtgtagttgcaacgacacgagcgcgacatgcgaggtgtccataacctgcatgcttacgcctcgattatcacaatcaaaagtagcgtgcggcagcagatccttaaaagtttccaccagcctcttcaaaactgcgccggttttaaattccgcttcgaacatttttagcagtgattctaattgcagctgctctttgatacaactaattttacgacgacgatgcgagcttttattcaaccgagcgtgcatgtttgcaatcgtgcaagcgttatcaatttttcattatcgtattgttgcacatcaacaggctggacaccacgttgaactcgccgcagttttgcggcaagttggacccgccgcgcatccaatgcaaactttccgacattctgttgcctacgaacgattgattctttgtccattgatcgaagcgagtgccttcgactttttcgtgtccagtgtggcttgttttaataaattctttgaaaatattgtcgggtgtattattaaatagcatgtatggtatgttgaagatgggataacgcttggcgtgcgggtcgtcatgatttccaccgcgcaccacatatttgcgctcaattttatcaaaattggactggcgagacaaaaacgagacgggcgacaggcatatttgggcgtgcgtaccatcttcggccatccactcggtcaggtcttcgctgcggttaaacacacctttctgaccgtgaatgccacatatttttattccttccaaatcgttggtggacgtgactatgactattttaagcataacgttgtcgccgttaaccaccatgctggcgtcgagtttttcaattttttgatttttaatttgtctaaagtaaacgtacactttgtaaacgttaaaattgccgttggtgcacgtttcaattttgtaccgtcggccgtcgtacacccaattaatctttgcgttgctcaccaacacaccggccatgtacagcacaagtccgtcgtctagcgcaacgtaatttttgtcgctactattcgtaaactttactaaacacgactgcttggggccgaccacaagcttgcccttcaatttgttcactttgttgttgtataaacaaatgggcagcgcaatgtgcggaatgtacggatcttcggcggtcatgagtttattgtctcgcaccaacgtccacaatttaaacattttattgttgagcaaaatggacttgtttaccgccacagagtagccatttggtaaacccgatacgcaattttcctctttgtactcaaacacgggcatggcattctttagattggttagggacacaatcaatttgggtacgggcgtggtatgaaataaatgtataaaattacgataataatactgctccaacttggacatgagcgatttgacgtcatcgttttctacgatcgtacactgaataatgggattatagtatatagaatgtttatagtggtattcgtagggtgtcaacaatacgttaatgtcggcttcgttgttcacccgcaacttttttttgatgcatatcattccttcgtgatgattaacgtaaagtattctgtctgtaatcttcaattcgatgggcgccatgtttcttttcatagtgtacacgataaacgacgtgtttgattttaaacattttaaatttgtgggtctatcattaaacgcgatcagcaacgagtcgtcttgaacgtcgttgaggtcgtccacgaacgcgaccagattgtgttttagcaaatattgaaatttttgcgcaaccatttcgtagtccacgttgggcaaacatgcgttgcggcaaaggaaaaactttttgcccgccacggtcatttcgccgtgaaaaaaactgccaataaatttcacaaaatcctttttttgcttcaacattttctggcgcatgctgtcgttggtgattcgcgccacctcgttgccgacgcgatattttaacacgggcaacgaaatttcaatattgttattgctgctgttgtcctgttgattgggaaagactttgcgttgcttgctaaaagttttcgatacgcaatatatgagacgcccgttgactatacaatcgacaatctttttcgactctttgttgtacaagacgctttgaattttacgacgcttgttcgccaccgtgtacgcgtcgtcgtcggccgtcttgtcgagaactcgttgatagttttgcaaaattgtcgaagttaataacagttctatcaaataggcgtgcttgtatacaattttgttggccaaactgtctatagaatagtttatgtcgtgattcataataatttttatgtgttccacgagttgttgcttgtgaagcgtgttgtattcgaagagaaaatcgagcggtttccatttgccgctgttggccagatatgtttccagcacagaatttaaatcttccgtcactacgtaatcgctagcgtacacgtctcgagcaaacaggacgtcgtcttgtttgtcgtaaactagttggattgcgcgattgatgtgcttctcttgatccacgttgccgtacaaaaacatgcgtttgcaatgtttggcgtatagcttgtcgtagaaattgtgcaccaaaacgttgttgttcatcattatgttgggaaaactcaaaaatctgccgtccagcataaaagttccgttaatattgttgtttgcgtcgacatcgtccgtttctctaaattgcttgtctaagcgcgtgccgaatataacgggcacacatttatgcattacgcaactgagctgttcattaagagcgcaacacaaataagacttgcgttcttgaatagcgcaaaaaagcatacgttcattgctgtttgtagcgcaatcaaaagtatattttaatttgtatttattttcaattctatcgtacaactcgttgaaatcttgaaccacgtccgtcatcgtgaagcgattactgcgcactaattatgtctaaacgtgttcgtgaacggtcggttgtttcggatgaaacggccaaacgcattcgacaaaacgaacactgtcatgccaaaaatgaatcttttttggggttttgcaacttggaagaaattgattattatcaatgtttaaaaatgcaatacgttccggaccaaaagtttgacaacgattttattttaacagtgtacagaatggccaacgtggtgacgaaacaagttagaccgtataacagtatcgacgaaaagcaccattacaacacggtgcgtaacgtgttgattttaataaaaaatgcgcgtttagtgcttagtaatagtgtcaaaaagcaatactatgacgatgtgttaaaattgaaaaaaaatacagacttggaatcgtacgatccattgattacggtctttttacaaattggcgaatctgtaaatgaagaaatacaaaaactcagaaaagctttggtcaatttttttactaataaacccgacaagtcggatataaacaacccagatgtagtttcgtatcaatttatttttggcagagtacaaaaattgtataacagggcaattaaacaaaaaactaaaactataattgtaaaacgtcctacaactatgaacagaattcaaatagattggaaaactctttccgaagacgaacaaaaaatgactagacaagaaattgccgaaaaaattgtaaagccttgttttgagcaatttggcactatattacacatatacgtatgtcctttaaaacacaaccgaattattgtcgagtatgcaaactcagagtcggtacaaaaagccatgactgtaaatgacgacactcgatttacagttacagagttttccgtggttcagtactacaacgtggccaaaacagaaatggtgaaccagcgaattgacataataagcaaggacattgaggatttaagaaacgctttaaaatcttacacataaattaaaatatcgaacaaaggaaaaaaacaattgtaacaaaaataatttacattaaaatttacaagtttttttctagtgtcgtacttttttacaatgcgtctgttgtccgtcgagcattgcaaacatattgtggacggcgcaaaatagcaaacaaaaggcacgtccgcgctctcccacgctattctaaaacgatgaatccatattaatttttcattgtcgccaaacgtcgctccgctggcctccttccaataacaaatactcagaaacacaaacatgtacaattgctgtcgcggcgttaattgtcgctgtttttccaaatagtctattatgggaaacaaacacttgtcacaacacaaatactcgttaattgtcacaaccgacaagcacatttggcaaaatgcgtcgcaatttttgtacggacgagattctatgcgaagttcgttgtccatgacgtcttgggtccactttttcaacaagacacttttatatttgtgatttgtacaactttggtacgtgttagagtgtttttgataagctttgataagtttaaaactgttggagtaaggccacgtcattatgttctgcaccttttgtttaaaagacagaaattactatatgttcaaactatttaaagattattggccaacgtgcacgacagaatgccagatatgtcttgagaaaattgacgataacgggggcatagtggcaatgcccgacactggcatgttaaacttggaaaagatgtttcacgaacaatgtattcagcgttggcgtcgcgaacatactcgagatccctttaatcgtgttataaaatattattttaactttcccccaaaaacactagaggagtgcaacgtgatgcttcgagaaactaaagggtttataggcgatcacgaaattgatcgcgtttacaaacgcgtttatcaacgcgttacacaggaagacgccctggacattgaactcgattttaggcatttttttaaaatgcaatcatgacgaacgtatggttcgcgacggacgtcaacctgatcaattgtgtactgaaagataatttatttttgatagataataattacattattttaaatgtgttcgaccaagaaaccgatcaagttagacctctgtgcctcggtgaaattaacgcccttcaaaccgatgcggccgcccaagccgatgcaatgctggatacatcctcgacgagcgaattgcaaagtaacgcgtccacgtaacaattattcagatcccgataacgaaaacgacatgttgcacatgaccgtgttaaacagcgtgtttttgaacgagcacgcgaaattgtattatcggcacttgttgcgcaacgatcaagccgaggcgagaaaaacaattctcaacgccgacagcgtgtacgagtgcatgttaattagaccaattcgtacggaacattttagaagcgtcgacgaggctggcgaacacaacatgagcgttttaaagatcatcatcgatgcggtcatcaagtacattggcaaactggccgacgacgagtacattttgatagcggaccgcatgtatgtcgatttaatctattccgaatttagggccattattttgcctcaaagcgcgtacattatcaaaggagattacgcagaaagcgatagtgaaagcgggcaaagtgtcgacgtttgtaatgaactcgaatatccttggaaattaattacggcgaacaattgtattgtttctacggacgagtcacgtcagtcgcaatacatttatcgcacttttcttttgtacaatacagtcttgaccgcaattcttaaacaaaacaatccattcgacgtaattgccgaaaatacttctatttcaattatagtcaggaatttgggcagctgtccaaacaataaagatcgggtaaagtgctgcgatcttaattacggcggcgtcccgccgggacatgtcatgtgcccgccgcgtgagatcaccaaaaaagtttttcattacgcaaagtgggttcgaaatcccaacaagtacaaacgatacagcgagttaatcgcgcgccaatcagaaaccggcggcggatctgcgagtttacgcgaaaacgtaaacaaccagctacacgctcgagatgtgtctcaattacatttattggattgggaaaactttatgggtgaattcagcagttattttggtctgcacgcacacaacgtgtagcatcgccagtatttaacagctgacctatttgttaaacaagcattcttatctcaataattggtccgacgtggtgacaattgtatccacaatcatgaaaaaagtagcgcttggaaaaattatcgaaaacacagtagaaagcaaatataaaagcaacagtgtgtcgtcgtcattgtcaacgggcgccagtgcaaaattgagtttaagcgaatattacaaaacttttgaagcaaataaagtgggccagcacactacgtacgacgtggtcggcaagcgagattacacgaaatttgacaaattggtgaaaaaatattgacatgctgcgatcaatcatgcgacgtttcaagagtacaaacaatctcagcaaaaaaccctccgattattatgtagtgttatgtccaaagtgttattttgtgacgtcggccgaagtgagcgtggctgaatacatagaaatgcataaaaattttaacacgaaattcgccgatcggtgccctaacgattttattgtgaccaactctaaaagttggaataatcatgaaaattgttctgccctattttaccctctgtgttaataaagtttgttgtttgtattttgtggttttatttatttacgctagatattgggtttaaggttcttagaaatagagttgtattttccctaccaaaagggatttgagcttcatataaatacaattttcgctcgacaagcggtttatttcactcggaggtattatatcaggcagtcgaacgtgcgcgatgaaacatcccgtttacgctagatatttggagtttgatgatgtagtgttagatttgactagtttaatatttttagagtttgataacgctcaaaatgaagagtacattatttttatgaatgtaaaaaaggcgttttacaaaaactttcacattacttgtgatttgtcgcttgaaacgctgaccgtgttggtgtacgaaaaagctcgcctaattgtgaaacaaatggagtttgagcagccgccaaactttgttaattttatcagtttcaacgcgaccgacaacgacaactccatgataatagacttgtgttccgacgcgcgcataatcgtggccaagaagctgacgcccgacgaaacgtatcatcagcgcgtgtccggatttttggattttcaaaaacgtaactgcatacctcggcccccaatcgagtcggacccaaaagtgcgagacgccttggatcgtgaactagaaataaaactatacaagtagaaaaaaattaatttattaatagttgtaataattatcttcgtcctcatcttcgctggtgtcataatgcggtggtgtgtttgtgttttgttttaatcgtttgcgcgtcgacaccacttcgccgataggaaattttttggatttcgcattaaatgcccgcttagcgacgcgccgtttacgactactaaacatgttgacgcgctcgtcgtcttcagtgtcataatccgtgctagtgttttcgttgttattttctatgagacgatcgtttgatttagttttcgtagaattgtccgcgttatcgtcgctttcgtcgatgtcgtccctaactatctcgtaggcggctttgcgcggaatccaagaattttgcaatgtatctattttaacgtacttttcttcgagcgcttttctagctttatgcatagcaatgtcttcgtcgccgccgttcattttatgatactttgtaaacgtctcgacgaataactttttggcgcgaggaggcattttttcattgtataacatatcgggaatttgatacattgtaattagaattaagcaagttcgtcttcggttgtactgtattcggtttctgtatctgtagtggaatcctctgtactagtagtagtgtcgctattgttggcgtcaggccttggctgccatttaccgtctatcaacatgtattttttcctaacagcacaacatgctagcttggtagctatctgtgtcgacttatatttttgtaaactacgatcgtagaatttttcaaatatcctcttaccgttatagggaaggttttgataatatttaggcaacatatcaataaaagacaatataaaaactttgtgtttgtgttttatttatcacataaaatggacgtctggcaagaatcacaaccaatattagtgttttttttcttacattacgagattcaacttgatactaaaattaattattaattaaattaaattaaattttgaagcattttttcgctatcgttttcagactcaaaattatcgacgctatcgctatgaaaagcgtaatatttgttggctttgagatattctatattttgctcatttttaacaataaacacgcgactcttttcgtcgcgtctcaccataacaccgtttttacaaatggaaatgtatttgtaaaacggcaacagagcgtcgcgagtttttttaagtaacagcttttgctccgctgtggcggccacaaatatttttacgggcccgtcgtaattaatgtttaaattaaaatttttaagtcgacgctcgcgcgacttggtttgccattctttagcgcgcgtcgcgtcacacagcttggccacaatgtggtttttgtcaaacgaagattctatgacgtgtttaaagtttaggtcgagtaaagcgcaaatcttttttaaataatagtttctaatttttttattattcagcctgctgtcgtgaataccgtatatctcaacgctgtctgtgagattgtcgtattctagcctttttagtttttcgctcatcgacttgatattgtccgacacattttcgtcgatttgcgttttgatcaacgacttgagcagagacacgttaatcaactgttcaaattgatccatattaactatatcaacccgatgcgtatatggtgcgtaaaatatattttttaaccctcttatactttgcactctgcgttaatacgcgttcgtgtacagacgtaatcatgttttcttttttggataaaactcctactgagtttgacctcatattagaccctcacaagttgcaaaacgtggcattttttaccaatgaagaatttaaagttattttaaaaaatttcatcacagatttaaagaagaaccaaaaattaaattatttcaacagtttaatcgaccaattaatcaacgtgtacacagacgcgtcggtgaaaaacacgcagcccgacgtgttggctaaaattatcaaatcaacttgtgttatagtcacagatttgccgtccaacgtgtttctcaaaaagttgaagaccaacaagtttacagacactattaattatttaattttgccccactttattttgtgggatcacaattttgttatatttttaaacaaagctttcaattctaaacatgaaaacaatctggttgacatttcgggcgctctgcagaaaatcaaacttacacacggtgtcatcaaagatcagttgcagagcaaaaacgggtacgcggtccaatacttgtactcgacgtttctcaacacggcctcgttctacgccaacgtgcaatgtttaaatggtgtcaacgaaattatgccgccgcggagcagcgtaaagcgctattatggacgtgatgtggacaacgtgcgtgcatggaccacgcgtcatcccaacattagccagctgagtacgcaagtctcggacgtccacattaacgagtcatctaccgactggaatgtaaaagtgggtctgggaatatttcccggcgctaacacagactgcgacggtgacaaaaaaattattacatttttacccaaacctaattccctaatcgactcggaatgccttttgtacggcgaccctcggtttaatttcatttgctttgacaaaaaccgtttgtcgtttgtgtcacaacaaatttattatttgtacaaaaatattgacgcaatggaggcgttgtttaaatctacaccattggtttacgcgctgtggcaaaaacataaacatgagcagtttgcacagaggctagagatgttgttgcgtgatttttgcttaattgccagttcaaacgctagttatttactttttaaacagcttacacagctcatagctaacgaagaaatggtgtgcggagatgaagaaatattcaatttaggcggccaatttgtagacatgattaaaagcggtgctaaaggcagtcaaaatctgattaaaagcacgcaacaataccgacagactttaaatacagatattgaaactgtgtcttcacgagccaccaccagtttaaatagttacatatcttctcacaataaggtaaaagtgtgtggcgccgacatatatcataacacggttgtgttacagagcgtgtttattaaaaataactatgtttgttacaaaaacgacgaacgtacaatcatgaatatttgcgctttgccctctgagtttctgtttccagaacatttgctcgacatgttcattgaatgataatataaatagagcgcatttgattgcatgcaatcagtgttttattaattttagagcaacatgtacgataaatttatgatctatcttcacttgaatgggctgcacggagaagcaaaatactacaaatatttaatgtctcaaatggattttgaaaatcaagtagccgatgaaatcaagcggttttgtgaaactcgtctgaaaccggcaatcagttgcaacactttaactgcggaaagtctcaatacgctcgtagacagcgtagtctgcaaaaatggactgttaaatccttacgccaaagaagtacagtttgctttgcaatatctttttgacgatgacgaaatatccaaacgagatcaagatggctttaaactatttttattacataattatgacaggtgtgaaaatatggaagaatattttttaattaacaattttagcatagcagactacgaatttgaagacatgtttgaaattgttcgtattgattgtagagatctgttattacttcttgctaaatataatatgtaattaaaattttgtttgttttattaaaatcctggattaaaaaatgacgaataatttgatttgcgtgcacgccaacaagattcttcgtcattatgatcaatgcgtgcatcaagtttatgcttttgtaattggcttctgaccactttagccatttgagcgtatctgcattcgtcgtctagagtttcaaacaccagatcggcgcaattataaaatccttcacccacgggatctatgcgctgccaacgcacatacattacaaattgatttgacctgtacggtattactacgggtatagaatagactagactgttgtcacataatgaatcgcccggatttggaattaaatttgaatcgttaccacctatgtattctaattcgttccaagttattggattgcgacgatcccagtttgatttagtaataaacacttcaaaataactgggctcgtgtatggctgttggacaaaaatgaacattcatctgataaaccggttgatagcgatttaaatatagcgtatttggcctccagttgttaaaaggttcgtccattccgcttttatcaccaaacacagaattgcgatcgtttgaaccggcaccgcaaagtgtgtgcggcacaaccctttgttcgattaggtcaaaatcgtcataattaggaccggccacagccgcgtattccatatactgttgaaacatgtattgcgctgtggaagcggccgccccggattctaaatcgagagctcgatatttataatagactgatttgtaagcattgcggcacgcggcgtcgggaatgttatcgccattgtcgggccaataaaagtttccatctttaaaacatttatattgacgggccgtcggcacggacaaatagccgtgagagcgcactgccggcgcgtgaatcgcagcaaacaatgcaattaataatgcaatcattatgattatacttatagaacactaatcggaataataaccgctgtcgtaatcttggtcaaaaacgttatgttgaaacataataacaccttacagtaacatacaataaaacaacatagtatcgtatataattataaactttattttttcattttatacaaacaaaatttatacgtattgttagcacattgagtgtcattttcgctgtctgaactatcacaatcatcgtcatcatcatcatcattgtcatcgtcgtcgtcacgtttgcgtttgacactgcattttttttggttaattttcactaacactggttcttttcgatcgtacaattgattctgcatgtacttttgcatgatcgcggtaaaacactttgcaattttatccttttgttcgtcgccaaatatttccagcaactcgttcataaatgtgcacaaaatgcccatgtgttttatccagctgattcgcattttcactggatcgaacaaacgcaaggggtacgctttttctgttaccttgccttcgatgtctatcaaaaggtacgggatacgatctccgttgccgggcacaaaatccgtgcctttgttaaccaaaatttctctacaatgcctagccaccgtaatcacgcgtcttttgggtgacggaccctcattatcgtcagttgatttgcgttttttgcccgggttatcgttataggtcatactaaagctgtagtcggtcaacgattttgatttggcaaactcatcatagtattcataaaaactagtctgtaaactttgcaaacatttgtccatgtccaaatgacgcaatatttgttccactgccgtcctaaacgcgattctcataaaaacgggcatatcctttttaactaaccaacccttgtatacgattttattctcactgttgagatagcaatattttttcttttttaatagtattaaaactttcattaaattttcaaatgccattttgtaaccgtccgtgaatgagttattaacgcgtgtctcaacatgtgtgcatatttgttttaatgtgtcggtttcgttggatatttcgttatagttaaatgtgggcaaaacaaatgtagaatctgtgtcgccgtacacaactttaaaagtgatgctgcccagattgaatttttctaaaatctcagggtcgttgctcaaaccttcaatcagagaaatggccagccgcaactgattgcgaccaactctagtgatgtagtttgcaagcactttgtaaaaaatgccataataaccgtatatgctattggcggtgcgcttcacggaattttgtttttgatcgtacagatcgtacaagaatgccgattcgctttgattgtcgcgattctttttaaatttgcacctttcgcttaacaattttaatagcaatttaacaactattgcacgcgaattgtggttcaaatacacgttgccgtcttcgcataaaattaaattggacaaacaagcacaaatggctatcattatagtcaagtacaaagaattaaaatcgagagaaaacgcgttcttgtaaatgcctgcacgaggttttaacactttgccgcctttgtacttgaccgtttgattggcgggtcccaaattgatggcatctttaggtatgttttttagaggtatcaattttcttttgagattagaaatacccgctgcggctttgtcggctttgaattggcccgatattattgacagatcgtttttgttaaaaaaatacgggtcaggctcctctttgccggtgctctcgttaatgcgcgtgtttgtgatggctgcgtaaaagcacgccacgctaatcaaatgcgaaatattacatatcacgtcgtctgtacacaaacgatgcaatatacattgcgaatatacagaatcggccattttcaatttgacaaacaattttatcggcaacatgcaatcctgcacgttgtacttggcaatcacgtccagccgtcgagtgttgtacatcttgaccatttcggtccaaggcaaatcgattttgttttcacccaaatagtaactactgattgtgttcaattgaaagttttcaactttatgctgattagaatcgctgctgaaaaatttatacaaatcaatgtgaatgtaatagttaaaataatacgtgtccactttgttgcccaacttgtttataaacagctttgtcgtcggcgccgcagccggcaaatcgtaacgctttaatagcattttggttttattcaatcgtccaagtatatagggcagatcaaatacgtctccgttaaaatccaaaatcacatcgggatttgtaatttttatcatgtcaaaaaacgctgtaatcatgtcgatttcattttgaaacatgaccacatacgtgtcatcgtcataggtctctggaatctgggtcggcagcttgtgatacataaaacaaaattttgcatactcgtcgtttttgtacaccacaaatcctatagacattatgcaatcaaccgatgctttcgacatgttgtggccgtccgaatgagtctcaatgtcatagcacgacaaaacgggcatgatgccgctggttaaagtcatttcatcgaccaactcaaagtcttcattaaaatgttgcaaattaaacatgcgcgtcgtcgatccaccgacatagttattttggcagcgttgtgttttcttgaatcgcatataggcgccttccacaaacggcgtttgcatgtgtacgcgattaacgttgtgaagaaacttgtccaaacacgccgcgttgtccgatggcgctgctttgtttctttcgtatttaatcacgtttatcttgttcaaataatttccttccacgcccggcgccacaaacgtggtgtagctgatgcacttgttgcggcaagacggaaatatgtgcttgtcgtagcattgtttgtaagaatacaaatttagttttactttaaagtaaaactgcagcactcgttctttgatatttgtattacaaaatgcaaacaagcaaccttgtttttcatcgtaatgcaaacgaatgatacgaaacgtatcggctgaagtaatattgaattctcctggttttgcatattctgcaaagcgcgttttgagttcattgtaaggatatattttcatttttaaatatgcagcgatggcccaaatatggaggcacagacgtcaacacgcgcactgtacacgatttgttaaacaccataaacaccatgagtgctcgaatcaaaactctggagcggtatgagcacgctttgcgagagattcacaaagtcgttgtaattttgaaaccgtccgcgaacacacatagctttgaacccgacgctctgccggcgttgattatgcaatttttatcggatttcgccggccgagatatcaacacgttgacgcacaacatcaactacaagtacgattacaattatccgccggcgcccgtgcccgcgatgcaaccaccgccaccgcctcctcaaccccccgcgccacctcaaccaccgtattacaacaattatccgtattatccgccgtatccgttttcgacaccgccgccaacacagccgccagaatcgaacgtcgcgggcgtcggcggctcgcaaagtttgaatcaaatcacgttgactaacgaggaggagtctgaactggcggctttatttaaaaacatgcaaacgaacatgacttgggaacttgttcaaaatttcgttgaagtgttaatcaggatcgtacgcgtgcacgtagtaaacaacgtgaccatgattaacgttatatcgtctataacttccgttcgaacattaattgattacaattttacagaatttattagatgcgtataccaaaaaacaaacatacgttttgcaatagatcagtatctgtgcactaacatagttacgtttatagatttttttactagagtcttttatttggtgatgcgaacaaattttcagttcaccacttttgaccaattgacccaatactctaacgaactttacacaagaattcaaacgagcatacttcaaagcgcggctcctctttctcctccgaccgtggaaacggtcaacagcgatatcgtcatttcaaatttgcaagaacaattaaaaagagaacgcgctttgatgcaacaaatcagcgagcaacatagaattgcaaacgaaagagtggaaactctgcaatcgcaatacgacgagttggatttaaagtataaagagatatttgaagacaaaagtgaattcgcacaacaaaaaagtgaaaacgtgcgaaaaattaaacaattagagagatccaacaaagaactcaacgacaccgtacagaaattgagagatgaaaatgccgaaagattgtctgaaatacaattgcaaaaaggcgatttggacgaatataaaaacatgaatcgccagttgaacgaggacatttataaactcaaaagaagaatagaatcgacatttgataaagattacgtcgaaaccttgaacgataaaattgaatcgttggaaaagcaattggatgataaacaaaatttaaaccgggaactaagaagcagcatttcaaaaatagacgaaactacacagaggtacaaacttgacgccaaagatattatggaactcaaacagtcggtatcgattaaagatcaagaaattgccatgaaaaacgctcaatatttagaattgagtgctatatatcaacaaactgtaaatgaattaactgcaactaaaaatgaattgtctcaagtcgcgacaaccaatcaaagtttatttgcagaaaatgaagaatctaaagtgcttttagaaggcacgttggcgtttatagatagcttttatcaaataattatgcagattgaaaaacctgattacgtgccgatttctaaaccacagcttacagcacaagaaagtatatatcaaacggattatatcaaagattggttgcaaaaattgaggtctaaactgtcaaacgccgacgttgccaatttgcaatcagtttccgaattgagtgatttaaaaagtcaaataatttctattgtaccacgaaatattgtaaatcgaattttaaaagaaaattataaagtaaaagtagaaaatgtcaatgcagaattactggaaagtgttgctgtcacaagtgctgtaagcgctttagtacagcaatatgaacgatcagaaaagcaaaacgttaaacttagacaagaattcgaaataaaattaaacgatttacaaagattattggagcaaaatcagactgattttgagtcaatatcagagtttatctcacgagatccggctttcaacagaaatttaaatgacgagcgattccaaaacttgaggcaacaatacgacgaaatgtctagtaaatattcagccttggaaacgactaaaattaaagagatggagtctattgcagatcaggctgtcaaatctgaaatgagtaaattaaacacacaactagatgaattaaactctttatttgttaaatataatcgtaaagctcaagacatatttgagtggaaaactagcatgcttaaaaggtacgaaacgttggcgcgaacaacagcggccagcgttcaaccaaacgtcgaatagaattacaaaaatttatattcattttcatcttcgtcatacttcaacagtcccaacacgttcatgttgtgattctcgccgttttcgacagttacgtaaatagttactttgattaaattatcttccagcagcattgagatttgattgaaatccgcacatagcttttgtagcgaatccgcttctttttttttatttgtgttgacgtagaaaacagatttgttccatttgcccaagtcggaagaggtagaacagtcatccgaatcggcaatgttcaactcgtcgcttttaaactgcacaataaacttgttatcgcccatgtcattttcttccaattcgctttttaacacatttacattgtacgaagcaacgtgtttgttcgatcgactaatgttgatctttgcgtttgtgcaattttgcaaatttgaatatgcttcgctttctttagcctcgcacaattcgatgcgcgtagagttgaccacgttccaattcatgtacacgtttgatccattaaaaatttgttgacactttatactgtaaatggtaaagatttggttttcattgtcttttaaatatttaaacacctcattgatgtcgtcagacccctttatattgttcttgaatagatttattagtgttttcgcattgacagaacattccacttgaaccacgtcgggatcgtcgttgagatttttgtacacaacctcaaaaacaactttgtacaaaccgctgttgattttcttgtagataaatttgtactttacaataatattgacgccatcttcattttcaaaatgtttgttagtcaaatagtcgctcatgggggttgcagtttcaatttccatttcacattctttgtattcgttgatctgaatcatttgactaaactttgttttcacataatttaaactaatgtcatagcacttgccttcttccatgtctttgaaagattgcgaatcgccgtagtattcttgaattttgttgtcggacattattcgaaaagtgtaatggtattcattatcgatactcaacgtcattttgctcatcaatttaccactaatccttttgtaattttctctaatcttcttggggctactggccatagccatgcgttttataagcggctcaccgctactttctccagacaaagatcttttggtcgccatattgctgttgtcgatatgtgggaatctatccgatggcaaatactgaatggcgacgaaatcgaagtgtcgccagagcaccgttcgttagcgtggagggagttgattataaacgtggccagcaacacgccgctcgacaacacgttcagaacaatgtttcaaaaagccgattttgaaaatttcgactacaacacgccgattgtgtacaatttaaaaacaaaaactttaacaatgtacaacgagagaataagagcggctctgaacagacccgtccgatttaacgatcaaacggtcaatgttaatattgcgtacgtatttttgttctttatttgtatagttttgctgagcgtgttggccgtctttttcgacacaaacattgcgaccgacacgaagagtaaaaatgttgcagcaaaaattaaataaactcaaagatggtttgaacacgttcagcagcaagtcggtggtttgcgctcgctcaaaattatttgacaaacgcccaacgcgcagacctagatgttggcgaaaactatcagagatcgacaaaaagtttcacgtttgccgacacgttgacacgtttttggatttgtgcggcggaccgggcgagtttgccaactataccatgtcgttgaacccgctttgcaaagcgtatggcgtcacgttgacaaacaactcggtgtgcgtgtacaaaccgacagtgcgcaaacgcaaaaatttcacaaccattacggggcccgacaagtcaggcgacgtgtttgataaaaatgttgtatttgagattagcatcaagtgtggcaacgcgtgcgatctggtgttggcagatggctcggttgacgttaatggacgcgaaaacgaacaagaacgtctcaactttgatttgatcatgtgcgagacgcagctaattttaatttgcctgcgtcccggcggcaattgcgttttaaaagttttcgacgcgtttgaacacgaaacgatccaaatgctaaacaagtttgttaaccatttcgaaaaatgggttttatacaaaccgccttcttctcggcctgccaattccgaacgctatttaatttgtttcaataaattagttagaccgtattgtaacaattatgtcaacgagttggaaaaacagtttgaaaaatattatcgcatacaattaaaaaacttaaacaagttgataaacttgttgaaaatataacgtgtgtataaaaagccagcggcttcaaatcaggcatcattcaacatggattcgctagccaatttgtgcttgaaaaccctgccttacaagtttgagccgcctaagtttttacgaacaaaatattgcgacgcatgtcgctacagatttttaccaaaattttctgatgaaaaattttgtggacaatgcatatgcaacatatgcaacaatccaaaaaatatagattgtccatcatcatatatatcgaaaattaaaccgaagaaagaaaacaaagaaatatatattaccagcaacaagtttaataaaacgtgcaaaaacgaatgtaatcaacaatcaaaccggagatgtttaatttcctattttacaaatgaaagttgtaaagagctcaattgttgttggtttaataaaaactgttacatgtgtttggaatataaaaagaatttatacaatgtaaatttgtatacgattgatggtcattgtccttcgtttaaagccgtttgtttttcatgtataaaaagaatcaaaacgtgccaagtttgcaatcaacctttattgaaaatgtacaaagagaagcaagaagagcgtttgaagatgcagtcgctgtacgcaacgttggccgatgtagatttaaaaatattagacatttacgatgtcgacaattattctagaaaaatgatattgtgtgctcaatgtcatatatttgcacgctgtttttgtaccaataccatgcaatgtttttgtcctcgacagggttataagtgtgaatgtatatgccgacgatctaaatattttaaaaataatgtattgtgtgttaaaagtaaagcggcttgttttaataaaatgaaaataaaacgtgttccaaaatggaagcatagtgtagattatactttcaaaagtatatacaagttaataaatgtttaattttaaggatattgttatggaataaactataaaatgaatttgatgcaatttaattttttgatactttccacagacggtagattcagaacgatggcaaacatgtcgctagacaatgagtacaaacttgaattggccaaaacggggctgttttctcacaataacctgattaaatgtataggctgtcgcacgattttggacaagattaacgccaagcaaattaaacgacacacgtattcgaattattgcatatcgtcaaccaacgcgttgatgttcaatgaatcgatgagaaaaaaatcatttacgagttttaaaagctctcggcgtcagtttgcatcacaatccgtggtcgttgacatgttggctcgtcgcggcttctattattttggcaaagccggccatttgcgttgttccggatgccatatagtttttaaatataaaagcgtagacgacgcccaacgccggcacaaacaaaattgcaagtttctcaacgcaatagaagactattccgtcaatgaacaatttggcaaactcgatgttgcggaaaaagaaatactggctgccgatttgattcctccgcggctaagcgttaaaccttcggcgccgcccgccgaaccgctaactcaacaggtctccgaatgcaaagtttgttttgatagagaaaaatcggtgtgtttcatgccgtgccgtcacctggctgtgtgcacggaatgttcgcgtcggtgcaagcgttgttgtgtgtgcaacgcaaaaattatgcagcgcatcgaaacattacctcagtaaacattgcaaacgactacgacattctttaaaaataagctatatataaatattgcattgtatgacaaaaaaattattaacctactgcaaagtaaaacttgtaaaaggcttttcaaaaaaatttgcgagtttattttgtcgctgcgtcgtgtcgcatctaagcgacgaagacgacagcgacggtgatcgctattatcagtataataacaattgtaatttcatatacataaatattgtaaaataaaagacatattattgtacataatgttttattgtaattaaattaatacaccaatttaaacacatgttgatgttgttgtgaataatttttaaatttttacttttttcgtcaaacactatggcgttgctttcgattagttttttcgttagcatttcatctaaaaaatcaaactgtttgcccggcgcgtttagggattctatggtgtagtcgggcgtgtcgctgtttagatattggtccacttcgcgcattatgtccaagacgttgttctgcaaatgaatgagctttgtcaccacgtccacggacgtgttcatgtttcttttttgaaaactaaattgcaacaattgtacgtgtccactatacaattcggcttaatatactcgtcggcgcaatcgtatttgcaatccaatttcgtgttcaacaaattggtgatgatatctttgaacgtgcacgttttcaatttgtccttatcggccaacgcaagtttcaattcgctctgtaaagtttctaaaattttgtctttattgttgtcaaattcgtgcgtgttgcgttccaaccacaatttgaacggctcgtcgacaaaaatgctgcgcaacacctcgtacaactgtctgcctaacgtgtacacttgctcgtattctttcatgctgacctctttgctaacgtacattactaaaaaatctacaagtattttcaaacatttgtaataggcgacgtattttgatttaagttttaaaccgtccaccgtgtattcgtccacgttcgcatcgaccacttttcgattattatcgccgcttgttgccggcgcgtcggcctgttcggttttaactatatccggttcaatatttaaagtttcaaaagatttaatggcattcataaaatcatctttttgctttggcgtggtcaatggtaaatctatcgaggagttgtcgtccgtgtgctcttcgggcacgctgttcagacgtaacgtaatctttttgggatcgtcttcatcgggtatcaaatcggctttaattttattagaattgagcaacgacatggtggtcgcttgtaaatttaataaattaattaaagactgaaattgtatattgcacaaatttattttcatttttattgatcttactattaatacgctggcagttggtatgcttcatccatttttgtgactagaaaatttgctaaaaaactgagctcgtcctgtgttaaaacgttgtcgtccacgaatctatgcaatgtaaatgttacactgacattgtttaacaatgcatgtattaaaaaatcaacctgtcgcctactgagtttattagaagagtcgaccgtttctactagtttgtagattttgttattttcaatttcattgtttaaaaacatgttaactactcgtttgagtttaagcgaaaaatccttgtccggatagacttgttcgcacagccaattgctaagagtggttttgaccacggacaccttggtggtgaacgtcgtcgatttgaccagttcggtgaaaaagtttttcattaaattggacattttaacaaacacttatcaatctattgagctggtatttttgtttagaatcgcatcaagcgcttgctcgatctccaatttttttcggacgctcttagctttatgactcggtatgtcttctacggtagactcggtgttcttacttataatggccgggctgacgataataaacacgagaaacaatatgagcagatacaaaaagatgctgttttcctttttgtcatacactaggctaaatatggccagtgcgcccaacaacaaatataaattcatttttattcccttactctattcgttgcgatagtacaacaacgattctcccgacgaaccggacgaattgcgattatgctgcgcgtcgtcgtcgtcgttgttgttctcctcttcgctgctcgtttcgtctaaacctatattgtatttgttcaagtaatgtttggtgcttgcggaggattcgtggttcattaatttggccactttttgtaaaggcacgccgctattgtataggttactgctcaaataatgtcttatcatgttgctgcgcggccgttccatctcgacgcccgactcttcaaggagtcgcctgaaatctttgaagggcgtcgaggtgtttttagatatttgcaaaatggtcgggtttcgtgaataaatctcgcgtgccaattccaacggtttcattttgatgttgttgagtgtgttattacgactgcgttttcgctttaaattaatcgtgtcgctgtgcagttttcctcttttaattagcacgttgagatcgtccacgctgagttggcgcgcttcgttgattcgcatacccgtccctaacatgatgcaaaacactatcgcgcccctaattagaccgcggtcgtgaacataatcgctgttgagcattttaattttatcattaataaaatttaatatggtatctattacgtttttaagcattaaattcttttccttttccctgatatttttgagctccttgtcgcgcggcagcataaccatgcggggaattttgtattcgggcaagttcatcatgttggtgtaaaagtttatagtcaactgtagtgtttctttggtgaccgagcgaagttcgagcatgcgcctgcacagttcttggggatcaatgagaagtgtttggttttctatcgagtcaaactccttgtccaacgagtacgacatgtcttccaggtgaacatcgtctaccgagcagtacacaattttaatgaatcgagacttgtaactttttaaagtggtgggcgcaaacggtttggggaacatgtacttgctccacagactgttgtttttcacctcgtcgggcgtgcatcgttgccgatcggtggccaaatcgaacacggactcgaaccggggagcggattgaatttttattttccaagaattaaaattgttttcgttgcgaacattaaaaccgttcattgtggttaatcaaatttattaaaaacaaaaggagaatcggtgtcaatactatccgaatattgttgttgttctcttaatattacgaaataatatattacatacagcagtaagaataaagctataaaagcgactacactaattaaaattataattcccgccgacacgttgctcgtcgtgttgtcatagcccaccatgtcgtttattggcattttgtgaacgggctcgctaaattgttgcggttcgctggcagtatcgtcgttgagcgccaatttcaacgggatgtattccaccttttcgtggttgcccaaccgatagtagggcacgtccaaattcatgtttacaacttatttgctaacaggaatttatgcaacaaaagtggtttggctttgatgagacgcaatttgaaatacttgctgcatttacgcttaagattgtattccatgcgggcggcggtcttgtagtcgtacgcgctcgcgctgtgatacacgagccgtaaattggttgcgttgcgcaaacacttggcgccttgtttgttcgaatgctgttttatgcgtctgttaagattgctcgtgatgcccgtgtacaattttccattgtcttgccgcagaatgtacacgcaccacaccttgttggtgtacagagtcgtcgccatgattatgcagtgcgccctttcgtgttcggccgagtggcgttaggcgcagccgcggcaataatcgcgttggcgtccttgttgtaatttatttgttgaaaaataaaacgtcttagagtttcgttttggaacgccaattcggtcaagctctcctggcaagcgcttttggtcaaatgagcggccggcgaattgaccgcgttggcggccgacgttaagaaggtggcgttctggaacatgctgggctgcttgccggctcgcgtcgccagctcggccatgtaattgaatatgttggcagacgcagatagcggcgccaaaaacgcaacgttctcttttaaactcatgactcgcgccctgtttttttcgttcagcacgtagtggtagtaatcgccgccgccggcaaacagatcgtcaatcacggcgttgatcagatcgttgatcatgttgatgtgcggaaagcgacgcgactcgactgcgctctgtatgtttggcggcagagtggcgtgcttgagcaacagagtcatgtaattgttggccagctgctgattgaaaggtaacggaatgggaatgttgcacgtcaccgcttccgccaccatgtactggacggccagactgagttgtttggcggcctcggccaaagcgtctttgcccaacatatcagcgccaccgttgtaaaacttttgcgcgtacgccggcagcgaatttagcacaaacgatggctgaaatatatttgaatcgctcgacagggactcggccgcgttgctctgtcccaactctttttgcaaccgaatcaggtggcgtatcatggtttcctccgattcaaaccgctttaccacgtttacgctgattgggttcgtgtcgatgcacatgtcacgaatagtgtttataaaaagaatcatgagaggactaagttctgacatgtcattgcacctgtaatatctaataatcttttgaacaaaatccacacatttgttgtaccaaatagattcaccggcgtcgagcgtcggttctttgctcttgttgtacggtgcaatcgctaccgagtttgtgctgttgctgcggctcgtgtaatccatcctgttgtcgcgcgtggcgacggtcgtaggcaccgtcgccggcggcacgtacccgggcgcgttgtaagtttgcgcgctggtgaatatggccgttgccggattagagggatacctcagcggcggaggggtgttgtaataaaaattgccacgttcatctgtcatactttttatttgtactcttatgattacaaaactcaatatacggattacttataatatagttgttgtgacaaaaaagcgataataaaattaacaaaattatcaacaagttaatcatggaaaatttttcaacgttgaataacaacaacaaaatggcgcaggtcaacagcaccgtttgaaaactgacgcgccgacacaaaatgctttcgcaatttctaaaagccacattaaacgaattttcacctttgatataatcacgcagttcttttttacaacattcgtcgcacaaaattaacacctttataatgaggccgtcggtgtgtatcgtttgaaatgtccgcggttgactgcctggatgaaattcaaacgagtacccagtggacacgtgtatctgtgcaaaataatgggctaatatcgaggcgcccgtttttttaacctttacttttgatattttaataacattaatgttgttatttgcgtaatcagagtttttattgtggtgatcatcgtacaaataatgaagcaacagttcactatcgtatttaatcttgtttagcgttgtcaagtttttgtttcttaggcgttggagcgtctccgtcgtcgatattttcttcgaaatcgagtccaacaacgtcggcgtttccttcttgctcatcgatagcggcggcggaggcggcctctccgtcgtcgtcattctcggtttctacagtgcgtttgggcgacgacgtgtgtacagcagcgtccgtcttactattatcggaccgccaaatttttgtttgaaataacatttggcccttgttcaactttatttcggcgcagttaaacattattgcattaagatcatattcgccgttttgcaccaaattgcacaaaacaccatagttgccgcacgacactgtagaataggcgtttttgtacaacaatctgagttgcggcgagctagccaccttgataatatgggcgccaacgccccgtttttttaagtaatattcgtcttcaattataaaatctagtacgttttcatcttcactgttgatttgggcgttcacgatgatgtctggcgtaatgttgctcatgcttgccatttttcttataatagcgtttactttaatgtatttggcaatttattttgaatttgacgaaacgactttcaccaagcggctccaagtgatgactgaatatgtgaagcgcaccaacgcagacgaacccacacccgacgtaataggctacgtgtcggatattatgcaaaacacttatattgtaacgtggttcaacaccgtcgacctttccacctatcacgaaagcgtgcatgatgaccggattgaaatttttgatttcttaaatcaaaaatttcaacctgttgatcgaatcgtacacgatcgcgttagagcaaatgatgaaaatcccaacgagtttattttgagcggcgacaaggccgacgtgaccatgaaatgccccgcatattttaactttgattacgcacaactaaaatgtgttcccgtgccgccgtgcgacaacaagtctgccggtctttatcccatggacgagcgtttgctggacacgttggtgttgaaccaacacttggacaaagattattctaccaacgcgcacttgtatcatcccacgttctatcttaggtgttttgcaaacggagcgcacgcagtcgaagaatgtccagataattacacgtttgacgcggaaaccggccagtgtaaagttaacgaattgtgtgaaaacaggccagacggctatatactatcatactttccctccaatttgctcgtcaaccagtttatgcagtgcgtaaatgggcgccacgtggtgggcgaatgccccgcgaataaaatatttgatcgcaacttaatgtcgtgcgtggaagcgcatccgtgcgcgtttaacggcgccggacacacgtacataacggccgatatcggcgacacgcaatatttcaaatgtttgaataataacgagtcacaactgataacgtgcatcaaccggatcagaaactctgacaaccagtacgagtgttccggcgactccagatgcatagatttacccaacggtacgggccaacatgtattcaaacacgttgacgacgatatttcgtacaacagtggccaattggtgtgcgataattttgaagttatttccgacatcgaatgtgatcaatcaaacgtgtttgaaaacgcgttgtttatggacaaatttagattaaacatgcaattcccaactgaggtgtttgacggcaccgcgtgcgtgccagccaccgcggacaatgtcaactttttacgttccacgtttgccattgaaaatattccaaaccattatggcatcgacatgcaaacctccatgttgggcacgaccgaaatggttaaacagttggtttccaaagatttgtcgttaaacaacgacgccatctttgctcaatggcttttgtatgcgagagacaaagacgccatcgggcttaacccgttcaccggcgagcctatcgactgttttggagacaacttgtacgatgtgtttgacgctagacgcgcaaacatttgtaacgattcgggaacgagcgttttaaaaacgctcaattttggcgatggcgagtttttaaacgtattgagcagcacgctgaccggaaaagatgaggattatcgccaattttgtgctatatcctacgaaaacggccaaaaaatcgtagaaaacgaacattttcagcgacgtatattgacaaatatactacagtcggacgtttgtgccgacctatatactacactttaccaaaaatatactacactaaactctaaatatactacaactccacttcaatataaccacactctcgtaaaacggcccaaaaatatcgaaatatatggggcaaatacacgtttaaaaaacgctacgattccaaaaaacgctgcaactattccgcccgtgtttaatccctttgaaaaccagccaaataacaggcaaaacgattctattctacccctgtttaacccttttcaaacgaccgacgccgtatggtacagcgaaccaggtggcgacgacgaccattgggtagtggcgccgccaaccgcaccacctccaccgcccgagccagaaccagagccagaacccgagccagaacccgagccagagttaccgtcaccgctaatattagacaacaaagatttattttattcatgccactactcggttccgtttttcaagctaaccagttgtcatgcggaaaatgacgtcattattgatgctttaaacgagttacgcaacaacgttaaagtggacgctgattgcgaattggccaaagacctatcgcacgttttgaacgcgtacgcttatgtgggcaatgggattggttgtagatccgcgtacgacggagatgcgatagtggtaaaaaaagaagccgtgcctagtcacgtgtacgccaacctgaacacgcaatccaacgacggcgtcaaatacaaccgttggttgcacgtcaaaaacggccaatacatggcgtgtcccgaagaattgtacgataacaacgaatttaaatgtaacatagaatcggataaattatactatttggataatttacaagaagattccattgtataaacattttatgtcgaaaacaaatgacatcattccggatcatgatttacgcgtagaattctacttgtaaagcaagttaaaataagccgtgtgcaaaaatgacatcagacaaatgacatcatctacctatcatgatcatgttaataatcatgttttaaaatgacatcagcttatgactaataattgatcgtgcgttacaagtagaattctactcgtaaagcgagtttagttttgaaaaacaaatgagtcatcattaaacatgttaataatcgtgtataaaggatgacatcatccactaatcgtgcgttacaagtagaattctactcgtaaagcgagttcggttttgaaaaacaaatgacatcatttcttgattgtgttttacacgtagaattctactcgtaaagtatgttcagtttaaaaaacaaatgacatcattttacagatgacatcatttcttgattatgttttacaagtagaattctactcgtaaagcaagtttagttttaaaaaacaaatgacatcatctcttgattatgttttacaagtagaattctactcgtaaagcgagtttagttttgaaaaacaaatgacatcatctcttgattatgttttacaagtagaattctactcgtaaagcgagtttagttttcaaaaacaaatgacatcatcccttgatcatgcgttacaagtagaattctactcgtaaagcgagttgaattttgattacaaatattttgtttatgatagcaagtataaataaccgaacaaagttaaatttttttcatttacttgtcaccatgtttcgaatataccctaataacacaactgtgcccggttgtttagtgggtgacattattcaagttcgttataaagatgtatcacatattcgctttttgtcagattatttatctttgatgcctaacgttgcgattgtaaacgaatatggacctaacaaccagttagtaataaaacgcaaaaacaaatcgctgaaaagcttgcaagatttgtgtctggacaaaatagccgtttcgctcaagaaaccttttcgtcagttaaaatcgttaaatgctgtttgtttgatgcgagacattatattttcgctgggtttaccaattatttttaatccggctttgctacaaagaaaagtgccgcagcgcagcgtgggatatttcatgaattcaaaattggaaaggtttgccaattgtgatcggggtcatgtcgttgaagagaaacaattgcagagtaatttgtatatagattatttttgtatgatttgtggtttaaatgtttttaaaataaaagaataacaatttacacattgttttattacatggataatgttgtttgtttgacattaaaggttatcatggtgcaatgattaataataaaacaatattatgacattattttcctgttattttacaatataaaatcacaccaattgtgcaaagttttattatttgtttgtcgacggtcgaggggtcagcggcgtgtgcaacaataaaaaacatgaagctgttaacaattttgattttattttattcattttttatgaatttgcaagcgctaccagattaccatcaagcaaataggtgtgtgttgctgggaactcgcattggatggaacgatgacaatagccaagatcccaacgtatattggaaatggtgttaaataaaagtgaatatattttttataaaattttttatttaaaattccaagtaatccctgcaaacattaaacactgtaggtatttttaaatcttgccacatgcgaacaacgcacggcctgtcgtcgaacaccgctattacattatattttcctctgatatagttgttaaacaattttaattttaataaataatctttacaagtatcgtctgaaggcctcataaacaatttatatgatttaatatcaaaatacttttcaatccagtttcgagtgggctgttcacaaattacgcttctcccgctcataaacacgataattgcgtcgtggcaatttgccaaatacttaacgcaagtaataacgtctaagcgggcttcatcttgagcaactctattatcaaaatcataaaacgatctatttgtgggcaaagctactgtaccgtctaaatcacataatacagcgcggggaaatttgtcgccgacaggaacgtaatattcgaaattatttacctttagaaactttttatattgctttttaatagtttctggatttaatggaaatttatcagagcgtttataattgcgttcaagagccgtttccaaagaaacgtccatcaaacgcgttaaaaaatggtaattatgcgttgcggccattttttgccacatgtccaccgattgagtgttcaaattagtgtcgctgacaaccacgttggcaccacattttgcggcttttaaaaactgttcaatgcacattttggtaatttgttcttctttagtttgtctacatttccgcgattggttatagaaagcgttcagttttgtataatcgccgtttaaaaacaacttaacgcgcacgtcgtctctgttgatttctgtatagccttttaaacttttggcatacgtgcttttgcccgaacccgaaatgcctatcaacaccaacaattgttttgaagaaggcaatttaattgttggagcaagtttattatttaatgcctgcttagtcgatacaaattttataatatttttgatcattttaattttttcaggctcggttaattttaaaaattcgctctccacatcgatcgtttgtgctttacgacatctgtacgctaaacatttccacggcaaagtttgcaccagttcgttgaaacgctgttgattcaaagtcaaacccgacaccataatatttattgtagactcgttggtgaacgtgtttctagcatcaacgtacggtttaatgacactttttaaatgcgggaaaagagctagaaagtcatcgtgttcgccatttataacaagctgcgccaatttagtaggattttcagcacggctctgatttttgtgcatgttcaaatacacgtcgcttttaatcttgcatagtggcgcgttgtttttatcgtaaactacaaatccttcttccaaatttttcaactgggccgcgtgttcgacacattcttgcacagacgtaaactcgtaacatttggggtatttgcaaaacggcaaattggaacagtaaaaataatcgcccgtttcgttgtttctgcttgccaaataccacaacgttggctgttcatcgtaaacggttacaattctgttgtgtttgcttgttaactcaaacatgtgagtcgacgcgcagtctaaatattcgttacacaacgcttgaaattgattgtgggcctcgtcaagttgaagagcttgcaaaactaaacgtttaaacgtcacgtctgacacgcaaaggttttctgcaaaagcacttcctcgggtgctggcatgccattcgccgttgtacttgtagattttaattaaacttccgtcgattttttcgtaaaacttaaaattctccttcgattggaacagtttgtgatgagcatcttcgccgccgatattttgtagcaattcttgaaaattaaagaaacgatcgaaagaacgcgacacaacggcgtacgtgcggctgttaagaattaaaccgcgacattccacgaccacaggatgatctcgatcgcgttcaaacgattcgtaattaagaaccatcaaatcgtgttcggtataatttttaattttgactttaaacttgtcacaaagatttttcactccgccgtttgcaagtagacgcgaaacgtgcaacatgattgctgtttaataatgcataccaatgctaaactgtctattatataaagtgcagtgataactttgttatcaacgcgttcgatgccgacatatataaacgcaatgtaacagtttttgctagtaccatcgcatacaacattatgaatacaaggggttgtgttaataataataaaatgatatttatgaatgctttgggcttgcaacctcaaagtaaattgaaaattattgcacataaaatactagaaaaatgtaaacgtgacgcgtacacgcgtttcaagggcgtaaaggcgatcaagaatgaactaaaaacatacaatcttacgttgcaacaatacaacgaggcgctcaatcagtgcgctttaaacgatagccgatggcgcgacacaaataattggcatcacgatattgaagaaggtgtgaaaataaacaagagacatatatatagagttaattttaattctaaaacccaagaaattgaagaatattattacattaaagtagaatgttatgtaaacagttaattaatctacatttattgtaacatttgtggtaatagtggcgttggttatacatttatatgattgtaatgttgtgtactcgttttgtaataaatttttgtgtttaatcaattcaatatttttatttgataaaaccttattttcgctactcaatttggcgtttttagacgcaagttttgcgtaatcgtcattgagcgattttagcgccttttcagttgtaattcgtttcagttgcaattctttaaaagatttatgcatgttgttgtagtcgcttttaattttgtctaacttttcttgcatagaaacgcttgtttgttgtaatttgtctaaatctaattgttgtttaatgttgagctgcgtttgttcggcaatgtctacctgtagtttttttagtatcgcttgtgcttcagacagcatagtgtcgtcggcatttgcgttgttgtcttctgcgtcgtccaacagacttttttcaaacaacacactggccaaagaggccgcatcaaaattagcgtttattttattccattgtgcgacactcgacgcgctgcatttaatcacatccacaacgtttcggtttacgctgtaaacgttgaaatgcaaactttcaaccctacacaagggacatggtactttttttcgttttctaatcttgcgtatacacattgagcataattgatgtttgcacgtgtctagttctaatacgggtattatagtcaatctgtctattggttgcagaaaataatttttaatttctgcaaccgaaaaacaaatgttgcattgcaatttaacaaactccatttttagacggctattcctccacctgcttcgcctgcaacaccaggcgcaggacctgccactgcgccgccgcccagagtagcgttaggatttgctcttggtataaagtcgttgcgcaaaaagttgttttctgaattgattatttggtatcccaaaaacagcggaacgtacgtcgggtattcttcgtatccgctaagcgttctgtccagctcacgtgtgtcgccttcaaatttcaaaacgtttctaatttgcaaacgattgggttgacttctcataatgtcactgcttcttatcgggttgtacaactcggggccgtcgggcacagacgcgaccagacccgtttcgtcaattatacacgtggcgcaatttctaaacctcaattcctccgtgtcgatttgcaagtactcgggcgctactgcgcgtcgaatcaaattttgcaaaaatccactgtaattgttaaataattgatcgccagcaccgcctcgaagcgctcgggcgttggtcacgtcaaagaaacgcaattcgtctcgcgacacccgcgaacaaaacgtgttcgggtttgtggtgtccagaatgctttttgtagttgcgtaaacgctgtgtataacgcgttgcgtgttgcttgtgaaaccttcggtatattttagattgtcgcatatagtgttaactgcgttttcgttgttatatatcaaatgaaagattagctgttcggcttgcatcatactgtttagattaaacacgtcttggtaattggttgcgcttggaattaaaattcgcttgatacctctttctttatttccaactaaatgcctagcgatcgtcattttgaattgattgtcgtcttcgtcgaaaatgggcaaaaccatttttgacattttaaaacgttttatgaggtggttgttgcaaataaaccatccatcgtcatgatacgcgtcgggcgaacacggcgatttgtatgttatgcacgcgtcgaacgacacgatggacgcgaaaatgcagcgattaactctcatttgtcgcggcgccatacccacgggcactagcgccatattgttgccgttataaatatggactacggcgattttgtgattgagaaagaaatctcttattcaataaattttagccaagatttgttgtataaaattttaaattcttatattgttcctaattattcgctggcacaacaatatttcgatttgtacgacgaaaacggctttcgcactcgtatacctattcagagcgcttgcaataacataatatcaagcgtgaaaaagactaattccaaacacaaaaaatttgtttattggcctaaagataccaacgcgttggtgccgttggtgtggagagaaagcaaagaaatcaaactgccttacaagactctttcgcacaacttgagtaaaataattaaagtgtacgtttaccaacacgataaaattgaaatcaaatttgaacatgtatatttttcgaaaagtgacattgatctatttgattccacgatggcgaacaagatatccaaactgctgactttgttggaaaatggggacgcttcagagacgctgcaaaactcgcaagtgggcagcgatgaaattttggcccgcatacgtctcgaatatgaatttgacgacgacgcgcccgacgacgcgcagctaaacgtgatgtgcaacataattgcggacatggaagcgttaaccgacgcgcaaaacatatcaccgttcgtgccgttgaccacgttgattgacaagatggcccctcgaaaatttgaacgggaacaaaaaatagtgtacggcgacgacgcgttcgacaacgcgtccgtaaaaaaatgggcgctcaaattggacggtatgcggggcagaggtctgtttatgcgcaatttttgcattattcaaaccgacgatatgcaattctacaaaaccaaaatggccaatctgtttgcgctaaacaacattgtggcctttcaatgcgaggttatggacaaacaaaagatttacattacagatttgctgcaagtgtttaaatacaaatacaacaatcgaacacagtacgaatgcggcgtgaacgcgtcatacgctatagatccggtgacggccatcgaatgtataaactacatgaacaacaacgtgcaaagcgtcacgttgaccgacacttgccccgcaattgaattacggtttcagcaattttttgatccaccgctacagcagagcaattacatgaccgtgtccgtggacgggtatgtcgtgctcgacaccgagttgagatacgtcaaatataaatggatgccaacaaccgagttagagtatgacgccgtgaataagtcgtttaacacactcaatgggccattgaacggtctcatgattttaaccgacttgccggagttactgcacgaaaacatttacgaatgtgtaatcacggacacgacaataaacgtgttgaaacatcgtcgcgaccgaatcgtgccaaattaaagcacgttaagcggatacaacgggcagtccgagctgttaaagtcaatacaaccatcgttaacaaacgaatacgcattgttgtgacagctgaggatataaaaaggaatagagaagtaattgcaatgaaatatcccgttacaattccacggcacagcgtatgttgctcgagttctatcagttgcacacaacggcctaagaaaatttattaatgcttcatttgtatctatattagaaggataatacataggttcgcccaaaggactgggagaaggcggcggcgaaggtgtaggtgtaggaggaataggagaaggcggcggcgaaggtgtaggtgttggaggaataggagaaggcggcggcgaaggtgtaggtgtaggaggaataggagaaggtggaggtgtaggtgtaggtgttggaggtataggtgttggaggaggtgtaggtgtaggtgttggaggtataggtgttggaggaggtgtaggcgaaggtggagaaggtgtaggagtaggtggaggtgtaggtaacggtacaattggtggagatgtaggtggtggtacaattggtggatttggatacaattcctgaatgtcgtctaatatttttaaagttaataaaattattataaataaatttaatattattattattattattatcacaataatgtaccacatgttgcttaaatataaaaattaaacaaagaatgttgtattattgcaaatttaacaattttttgtattctccccatgtcatgcgttcgtaatgagcgggcggttttttatttctttgtatccacttgtaatcgttaatgtggttgtgaaaagtcatactgacgtaggccattaaatttttcatgagcatattatttgacacaactgcaacatctgcgcctgccgtttcttgctggtacgaatcgacaaacgtaatgtctgtgccgtatttttctttgtcaagtgcaatttctataagctcaatgtggtaaatgatgaaacctttgacgttcatataatgatcgcggcacatggcgcactgtagtatgaaaaatacgttgtaaaatagcaccttcattgttttcaactgctgcatgacaaaatctaaactgcttttgtctcgcgtatacaccatatcgtcgatgatgagactgagaaagtgcatggtgtcccatatggtagtaaacgtgtaagtaaaactcttgggctggcacgaacgcaaattgagttctgtggttttgtccataaattctatgcgaaactgttgcaagtccatgtcgggggatgcgttaatggcccattcgatcaactgctgcacctcgtacttttgaatgtctttgtatttcatcaaacacgcaaaatggtataagtaagttgcttgcgaagacaacagtttggtgaggtgcgtcgatttagaggctcgcaaaaggtctatgagacgaaacgaatacaacagatagctgtctttgtaacgagaaaaaagcggcgtcagcggtatcatggcgactagcaaaacgatcgtgctgtacttgtgtcaggcgccggccacagcgtcgttgtacgttagcgcagacacggacgccgacgagcctattatttatttcgaaaatattacagaatgtcttacggacgaccaatgcgacaagtttacttattttgctgaactcaaacaggagcaagccttatttatgaaaaaagtatacaaacacttggtgcttaaaaacgagggtgcttttaacaaacaccacgtattgttcgatgcaatgattatgtataagacatatgtgcatttggtcgacgagtctgcgttcggaagcaacgttatcaactattgcgaacagtttatcacggccatttttgaaatttttacgctcagcagtaaaatcgtcgtggccgtgcccgtcaattgggaaaacgataatttaagtgtacttttgaaacatttgcacaacctaaatctcattggaattgaaattgtaaattaaaacaaatcatgtggggaatcgtgttacttatcgttttgctcatactgttttatctttattggacgaatgcattaaatttcaattccttaaccgagtcgtcgcccagtttagggcagagcagcgactcggtggaattagacgagaacaaacaattaaacgtaaagctgaataacggccgggtggccaacttgcgcatcgcacacggcgataataaattgagccaagtgtatattgccgaaaaaccgctatctatagacgacatagtcaaagagggctccaacaaggtgggcactaacagcgtttttctgggcaccgtatacgactatggaatcaaatcaccaaacgcggccagcacatctagtaatgtaaccatgacgcgcggcgccgcaaactttgatatcaaggaattcaagtccatgtttatcgtattcaagggtgtgacgcccactaaaactgtagaggacaatggcatgttgcgattcgaagtcgacaacatgattgtgtgtttgatcgaccccaacacggcgccgctgtccgaacgagaggtgcgcgaattgcgcaaatctaattgcactttggtgtacacaagaaacgcggcagctcagcaagttttattggaaaataactttaccgtcattaatgctgaacaaaccgcctatctcaaaaactataaatcatacagagaaatgaattaataaaacaaaaagtctatttatataatatattatttattaacatacaaaatttggtacactagtgttcaaatcgtttctgttcaacgccattgtcatgttataaaacacatttgtagttttattgtaattatttttaaatttatttttaatttgctgtaataaaacttgttcattaaatacaaaagactttgaactacttgcgtttatattctttttataattgtactgaacaaacgaggggtgcaaaaagtttttcaaatgctgcacggcaatacctatcatctcctccattttgtcctctcctattgtaatagtggcactgcgcaccgttttaatgtttagaatgtaaatgagcgcatacagcggactattgttggtgctcaagcacattaggttgtgcttatgcatagggtcgttgctcagcagcgttttgtatactacaaagcccgttttggggtcgcgtctgtacattagtacgtgcgacaaaaacaaacgcaccggcgtcacaagcgactcgtaatacatgctttctatcggaaactgtttggacttgatgtgttcgtacacggagccggcaaacttgacgctgtctacaaacttatggttcgtgtaaacaatcaaaaatctgtcttgtacaccgtcgtcataatcgtccacgtacagcggcttgttgttaacaattaacattttgtagttggcttcatactttagcagcccttggtattttctgctcttggaatcgctcttgctcgaatcggcatgcttcttaaagtacgactcgctgcattgtttcaactcgttgatagtgtacaactgcgagttgagtttgctcacttccttgtcgctcgtttccttgttggactctccgctgtggttgtcatcgtcaaacttgtgcatcaacaccaaatagtccaacagctcaaaaaacgacgacttgcccgaacccggttcgccgggcatgtaaatagccttctttccgtaatctacgggaatggccaaactagcggcgaaatgcatcaacataatcgcgttcgcgtgattaaaattggtgaagcgtttaaagtacaaatagccttcgacaatctttttcaaataattgtacgagtactccttcaagtccactttggacatgatgatgcgcatgtagaatcgagtcagccaagtgggcaaatcgtccgtgctgcgcgccaatatgattttgtcccaccacacattgtacttcttcaagatcattaacgcgtcggcgtggtgcgtgtaaaatttggaaatgttatccgattcttcaaactgaacatcgggttcacgtgcaacatcatcgcgcaattcggttaaaaacaaacgtttatcattaaacttgtccatcaacatgtcgacatattcgattttgtgaattgttcgatacaagtactgaataattttgttgtgttctttggaaaaaaactctccgtgttggttaacaaattcgctgttcgtgcgaatcaacgtggtcgacacgtacgttttgttagtaaaaattagcatccaaatcaattcgctcaattctgcatcgttaccgaacatgtccgccatcaagcagacttttagcgcttttctattgatctttattttcttgtagcatttgcattttggtcgagatcccgataccgttgaccgacacggtttgcattttaggttgtgcaacatgtcggaaaccctgttcttgtttacgtacagagcgagcgtaatcagattttcatcgtccaaattccacaaatcgcgaaacaggttgtttaacgcgactcgcatatcggcttggcatgtgttgcaattgcccatgtagttaactatggccgtgttagtttttagcatttttacatctcggcacattttggcgatgtgataagttctataaatgctgagctcgtcggcgctagtagatagcatgtaattaaacgcgtcctcgggcaaatacttttcgtcggtgggcttcttgaatgtctgcggcaacgtggtgcccaacaaaaatggacagctcgaatgaaagctgttggtgaacacgttgtacacaccgtgcgttgtcaagtacaagtatttccaattgttaaattttatgttgctcaacttgtaacaattgcttttggtcaatttgaataggtcatcctctttctttacaatttgataatgtttgccgttgaaaaccaaattgactccggtcactacgttttccaattttctaaagaatcctttacacacaatgtcaggcggcaagtttagcgccatcacattctcgtacgtgtacgcccacaattcatcgtgatccaaaatttcgtttttagccgactgagtcaaatatatcatgtagtgtatgccaaaataatagcccaacgatacgcacaatttggtatcgtcaaagtcaaaccaatgattgcaggccctattaaacactattttctcttgttttttgtaaggctcacatcgcttcaaagcttcattcaaagcttctttgtcgcaggcaaataatgattcacacaaaagttccaaaaacagtttgatgtcggtttctctgtacgagaaattttcgttcttggtcaatatcttccacagtacatagattaaaaaatcaaaatttttaaatttgcttttttcaaagtattgttgtagaaggtttggatcgttggctcgttcgtgggtcgccaaaactttaaccatgttctcgtgaattgctataagccccaaattgatttgcgtttgaatgtagtctgcattttcgctgctcgccgatataatgggtacgatgcgcggttttctggaacgcgtgtcgctcaagtccacgtcgtttttgtcaaaattgttgttctcgaacactctgaggcttttgaggttgacgttgacgatatgcttgtacttgggcaccgtaatgcattcctccaaattaatgtcgtccctaatgtaattgaaaaaatttttatccgaattgaccagctcgccattaactttgcacgtggccacagtgccgtcggccattttgagtataaacaagtcttcgtgagaatcgtcaaacttggtttttccatttacaaacagcgtttgcggcggatcgtgattcgtgcgcaggctgagctcgacgttgagaaaacatttagggtcaaacacaaacaaatccacagggcctagttttttgttgtgtatgattggtatcgtgggttcgatgacaattccaaattttatatttaaaaacagctgccatccgttaaaagagaaagcttgctttttgggccagttgggccaataatagtaatcgcccgcttgcacgcatttgttaatgtatccagggtcggtgctcttgaaaaaatcttcaaaattaatatacttttgtatgatgtcatagtgcttcttcaaaatgaaaggttttacaaaaatgcaaaaatcgttactttccaacacccagtcgtggccgtctaatgtttgagctgcgtgtttctctgcaggttcttcggtgtcttcgcaagatgcgcccatgtcgtgtttcgcgcacggaccgttaaagttgtttctaattgtgtttaagaactgttgaaagttgttgacgtactcaaacaatctacgtgttcctgttcgcgtgtttctaatgattaaatgatttgcatcttgcaagttgttaatctcgtacgttttgtcttgaggcacgtttttcaaaaaaaattgtaaaatgttgtcaatcatgttggctatcgtgtttgtacttttcgtgttaatttatttaataatttcgatcaaaaatcaccatccattcttacatagaatagaaacgctaatacaagatttcaacaacacattgttgtttggcgcgtatgtacagatttacgatttaagcacgcccgcccgcaccgaacgattgtttattattgcgcccgaaaatgtggtgttgtataattttaacaaaacgctctattattacttggactcggcgaacgtgttttgtcccaacgagtttagcgtgaccacgttcacgcaatccactattaaaacgatcaacgagacgggaatatatgccaccgcatgcacgccggtcagcagcttgacgctaattgaacattttgcaacattaaaaaataacgtgcccgatcacacgctcgttctcgatgtggtcgaccaacagattcagttttcaatactcgacattatcaattatttgatttacaatggctacgtggatttgttggccgaataacgcgtatatagacgcttgtacgttcatcgtagtaatcattttaatacatttgattgaactaaacatacatctgcaatgggtgaaagagtcactaaattttgcaatggaaaacggcgataaagaagacagcgacaatgaatagagtttatatttttatttaataaaatattgttcgtaatccataatgttttgtattatttcattgtgataatgttcccaatcttgcacgggggtggggcatcgtttgactttgacgtagaaatcgtacgcgtagttattagttggcagatcgtcgacaagtgtgatcgacttgaaaaagtttacatttttatcgctcaaatatttaattacaatttttggcgatttgggtatattgttgtcggatcgatgattgtgaatgtcaaaaacaaatttattttcaatgaaacgcttttttaaattgtaatctacaatagcgttgtgtgaattttgaactaaatcagagcgttcttcttgaacggtggaaccttcgctgataatgatatcaaaatagccttccaaatcgacgtctcgcatcgagtgtgctacatgatctctactgccatacgaccacaagactaaaacgcaacccatctcgtgcaactcctgcaagctgtcatacacaaacggatctcgaatctcaacttgctcctcttcggttatgagagtgctgtccaaatcaaacacgaccacgtgcggaaatccccacgtcaaagattcgcttttgagagagaccactttgtagtgtggcaatagaaaccattctttaagaaacgaatacattggcggtttgttgctaagcacgcacatgtggcccaacactggcgttttgaatgcgcgtttaatattgtgcctgatgtcgcgcatgtcgtcggcgggcgctttgaatatttgcatacagtaattgtaattgttttctatgatcttgcacagctgcgggtcgttgcaaaattgaaatattacatattcaaaaaatttatacttttcaaagccaaggtatttgaggtcggcgtactcgcttaaaacgagaacatgtcgtttgatgatggcgtcgttaaggcgcaaacagatccatttgctttgaagcgaggaggccataatgtacaaaaatggaccagttacgccttatttaaactgtttaaagagtttcgtataaacaaaaactactctaaactaatagatttcttaacagaaaattttcccaacaacgtcaaaaacaaaacgttcaacttttcgtctaccggccatctgtttcactcgttgcacgcgtacgtgcccagcgtcagtgatttggtgaaagagcgcaaacaaattcgattgcagacagaatatttggcaaagctgttcaacaacacaataaacgatttcaaactgtacactgagctgtacgagtttatcgaacggaccgaaggcgtcgattgctgttgtccgtgccagctattgcacaagagtctactcaacaccaaaaattacgtggaaaacttaaattgcaaactgtttgacataaagccgcccaaatttaaaaaaaaaccttttgacaacattctttacaagtattccctaaattacaaaagtttgttgttgaaaaataaggaaaaacataccagcactgggtgtacacgcaaaaagaaaatcaaacacaggcaaatattgaatgataaagttatttatttacaaaacagtaataaaaataaactatttgagcttagcgggcttagtttaaaatcttgcagacatgattttgtaacagtcgaaagccaaacgagggcaggcgacgaaatcgcttcgttcattcgctactgtcggctgtgtggaatgtctggttgttaatagtagcgtgttctgtaacttcggcgacctgtcgatgaacggctcctggatcttctgtatgtgcggggtctacccgggcggcgtctgtaacccgagcttctgcgcctgcgtgtcgaaccatatgtggtaccggttgaagaacggcgacggcgacgataaaccatgtttaaattgtgtaatttatgtagctgtaatttttaccttattaatattttttacgctttgcattcgacgactgaactcccaaatatatgtttaactcgtcttggtcgtttgaatttttgttgctgtgtttcctaatattttccatcaccttaaatatgttattgtaatcctcaatgttgaacttgcaattggacacggcatagttttccatagtcgtgtaaaacatggtattggctgcattgtaatacatccgactgagcgggtacggatctatgtgtttgagcagcctgttcaaaaactctgcatcgtcgcaaaacggaatttcggtaccgctgttgatgtattgttgcggctgcaacatttgtatcttttcgccgcgctcgatcaacaattcttcaagagtggtgcgtttgtcgcgctgtaaagccacgttttgtaacagcactattttcgcatatctcataatcggactgttgaaacagcgtgcaaacgacgaccgcataatatcgacggtcgtcaagtcgattgtggtcgaaggcatctccaacagagatcgcacggcgtccaacagcgtgtccgtttgaacctgcgtcatttgcggtctgcacgtgtagtcgtcaaacgtggtttcgagcagtttgaacaacgaatgatacttttccgatcgcagcaaaaatatcatggtcatgaccacgtcgctgattttgtattctgtagaactggtgctgttcaacgaatagtgatggattagtttgcgagcagcatttctgtatcggcgcatgttgatcaactcttcggaaggctgcgcgggcgcggcggcgttggctcgcgcaaacaaatttattacgggacgcggcgtaggctgcgcggacgctggcgcggcgacgacgtccgcgtttcccgccgcgtactgagacgctatggcagcgttgttatttaaaattgtgttttgcgatttgcgagccacgtgcatcataaaatttatcaacacgtcggtgttcaactgcacgctttgatgttcgtcgcagagcaaaggaaatagctggggccatatcgccaattgcataggctcgtctatttttaaccgcaatttgtttatttccaaatacaacgcgatagcgctcatcgtgaccgacgacgcacacttactctgtaactatcacttggatcgtgttgtcgtaaacgcttcccaaaaagtctaacacgttgaccgtttcgattctattcaacttaattgtggacgcgttggcttgcatcggttccaacagactgcgcgctccgacagattgagtagacaaaatttttaaactttccgtcttattgggcgtaatgtcgttgattaacaacgacgcagccgtttgagaggccgcagtgttgatggtttgcaacatgtcgacggccgccatttgcgtttgcgccgaaggtcttgctggcggcctgttgcggcggtttcttcgtgcttgcgacatgttgtcgtcagtgtccatatcggtatcatttattgaagcaatcatggttgagttcgataagcagagatatttcgttgtccaattggtacttggtaatgatgtgccttataaatgtttcgggcacaatcatttctgtcattagcacgttacaaatatctattttgatcaatttcaatttatgaattaacagattaatgttttcgtccgagtacttgctcatgatgaaacgacaaacgttgcggagttccaactccgctaccggatacgctttgttgggcaaactctctaaatagtgtctcaaataaaagccgatcaatacggtggacgctattttgttaacctttttcattttagtattgcggcccatttctatcatgaagtttttaaacggtagcaacagcctgtctccgttagcaacagtggagcagccgttgcattgcgcgctcaaaatactcaacacgcgctcgtgatcttcttggcgcaatccgacggttgcttttttgcattctttgacaaatggcacgcacatgtcgcgtttcgtgtacaaagaatacgctttgtcgcaaatcaagttatagaaaaattgcacaaatatctgcgtaatcaagttgttttcgttaataatgtcactttcgtttttgtaatcggttcgaagcaacacgtacaacatcagaggcatgccgaacatgggtcttaaaaaaatgtcccaaccattttgcaagcccgcgtcgagggtgctcagcgaggacgccaagtatttgcatttgcactcaaaacattgaattttgtttgcgggcttgcacgactgacacatgatcgcatccacgtcgggtgccggcgtcggattgtaatatttttgcaagtattgcataatggtcctaaaatggggtacctgtttgataaactcgtcgcgcaaaaatatcgaaaaaatgttttttacattgtgtatgttgtctgtgttgttggcttgattctcaaaactactctttatggaaacaatacatttgttaaattctgtgaaaaaagtaagacctttactgtccacgatcaagctttggttgaaatattttgaaaataaaaaacacaacgaatcgatttcatcttgtaacaattgcgcttcaaaacacacgttttcaaagcggtcgtaaatgttaaaccttaaactgtattgtaatctgtaagcgcacatggtgcattcgatataaccttataatatgaacgattccaattctctgttgattacgcgtttggcagcgcaaatactgtccagaaacatgcaaacggtggatgtgattgttgacgacaaaacgctcagtttggaagaaaaaatagacacgttgaccagcatggtgttggctgtaaatagcccgccgcaatcgccgccgcgggtaacatccagcgacctggccgcatcgatcattaaaaataacagcaaaatggtgggcaacgattttgaaatgcgatacaacgtgttgcgtatggccgtcgtttttgttaagcattatcccaagtattacaacgagacgaccgccggtttagttgccgaaatagaaagtaatctgttgcaatatcaaaattatgtaaaccaaggcaattatcagaacattgagggttacgatagtttattaaataaggcggaagagtgttatgttaaaattgatagactatttaaagagagcattaaaaaaatcatggacgacacggaagcgttcgaaagagaacaggaagcggagagattgagggccgaacaaactgccgcaaacgctcttctggagaggcgagcgcagacgtccgcagacgatgtcgttaatcgtgccgacgccaatattcccacggcatttagcgatccgcttccaggccccagcgcgccgcggtacatgtacgaaagttcagagtcggacacgtacatggaaaccgcccgacgtaccgccgaacattacaccgatcaggacaaagactacaacgcggcgtacactgccgacgagtacaattccctggtcaagacggttcttttgcgtttaatcgaaaaggcgctggccactctaaaaaatcggttgcacataacaactattgatcaattgaaaaagtttagagattatctgaatagcgatgctgatgctggagaatttcaaatatttttaaaccaggaagattgtgtgatactgaaaaatttgtcaaatttagcgtcaaagtttttcaacgttcgttgcgtggccgacacgttagaggtaatgttggaagcgcttcgcaataatattgagttggtgcagcctgaaagcgatgccgtacggcgaatagtcataaaaatgacgcaagaaattaaagattcgagcacgccgctgtacaacattgccatgtacaaaagcgattatgacgccataaaaaacaaaaacattaaaaccttgttcgacttgtacaacgacaggctgccaatcaatttcttggacacgtccgcaaccagtccagttcgcaaaacttccggcaagagatctgcggaagacgacttgttgccgactcgcagcagcaaacgtgccaatagacccgaaattaatgtaatatcgtcagaagacgagcaggaagatgatgacgttgaagatgtcgactacgaaaaagaaagtaaacgcagaaaattagaagacgaagattttctcaaattaaaagcattagaatttagcaaggacattgtcaacgaaaagcttcaaaaaattattgtggtcaccgacggtatgaaacggctgtacgaatactgcaactgcaaaaattctttagagactttaccgagcgccgctaactatggcagcttgctcaaaaggctaaacctgtacaatctcgatcatatcgaaatgaatgtaaatttttacgagttgctgtttccattgacactgtacaatgacaatgataacagtgacaaaacgctttctcatcaattggtaaattacatatttttggccagtaactattttcaaaactgcgctaaaaacttcaactatatgcgcgaaacttttaacgtgtttggcccgtttaaacaaatcgactttatggtcatgtttgttataaaatttaactttttatgcgacatgcgtaattttgccaaattaatcgacgagctggtgcccaacaaacagcccaacatgagaattcacagcgtgttggtcatgcgggataaaattgttaaactagcttttagtaatttacaatttcaaaccttttcaaagaaagacaagtcgcgcaacacaaaacatttgcaaagactaataatgttgatgaacgcaaactacaatgttatataataaaaaattataaaatatttttaatttttatttatattcagtacatttacacatattaacatattgtttatacaaattcttataatcattatgatttaaattgaattgttgtctaaacaaattaaacactttattaaacaataacttttcgttgtaattttttactttgcacatgttataacaaaaaattaaaattttcatcatgtctgatttgtctatggcgtcacagttgcttttaatgtaatcgcaagttaaccactcaaaaggacccttttctatttttaatttgtttaaatctttataatcagacttcagtttgtaaattagatttccacatcgaataataaatccttccagcgggctttggggaaacattaaagacttgaaatttaacctttctacaaaatcgttgtacaaatatttgtgacacggaatagtattaaaccccacgttagtcaacaactcttgcgcctccacaaagggcacaaactccccgccgtataattgaatttcgtaagcgtagtatttcaaactctctttctggtccacgtagttaattacgttaatgggtgtcgtttttgcgtcgtctttccaacccattaattcgccgtagacaataaaaccgtcattgaaccgcgcctgaagcgatcgcatgcacgtttctaaatcttttcgaatgcggtaataattcataaaattgccgtccggtctgtaagtgtttcttgacccgtacgtaattttattttggttgcaaatgattctgaaattacaaccgtccaacttttcttgaacaataatttctttgtcggccaacgtaccttttttaccttgatctagatgcgacacagatggataaatttgatacacaattttattctcatcttcgggcattacgggtccgcgttcatttaacgcgtacatgacaatgttgtggcgaatgtcggtgcgctccggcggttctggcacgtggtgcagtctgtcctgcaattgttgcttccattgttgaaaatattcggtccattcttgttgatactcgccgcgttgcatgagttttacgtacagttttaaaagtttgacattctttacaaataacgttagagtttcgtcgattttgtatcctccattatttttgtttaaatccaatacatttaaatcgttcactaccagttgattgtttttatccatcgtaatttttatctcatcgcccacgttgaacaacatgtttaaaattttggtggatttcggcgcacgtttataatctaaataatattcaacgtacacgtaattgaacatgagctgcaacaatcctttggcattgttcaaaattttgtatctcatcaaagtataaataattttcaccatcgacaccgtcatcaacttggttacaaactcgtacaattgcaagttttcaataccgtatttgtctttaaaatcttcacgtttactgaacatgcttaattcgggagattttccagtcaaaatgccaattaatcccgtgtacaagtcaacgtatttgacatcgttgcccgattcatcttttgcatgtcgatttttcaaaagctctttattgtcgataaatttttcaaaggtctctcgatcacatttagtgtaaatatggtagtcagtgtcgctgctttcgaccgcgtatcccttggcatggctgcccgtatcaatgcaaatgtacaccatgttagaatgtgctgcttactgtgcctgtatcaagccttatatacctcaaaatatttcacatttttgcatcatcgtaaaatatacatgcatataattgtgtacaaaatatgactcattaatcgatcgtgcgttacaagtagaattctactggtaaagcaagttcggttgtgagccgtgtgcaaaacatgacatcataactaatcatgtttataatcatgtgcaaaatatgacatcatccgacgattgtgttttacaagtagaattctactcgtaaagcgagtttaaaaattttgtgacgtcaatgaaacaacgtgtaatattttttacaatatttaagtgaaacattatgacttccaataattttgtggatgtggatacgtttgcaagacaattgattacagataaatgtagtgctctaatcaaaagtgcggatctgttgccggcaaacattttagagattgtagagaaggccagagacaagtattttgaggagccaactcaaaaaaactatgaatacattaaaaaattatttttacgaacaaaatatatggacgattcgatagattataaagattttaacagacgcatcctattgatagtttttaaattcgctttaaacaagagcaccaactactttccatcgtacaaagagatcatcgaggtggccattaaacgtttaaacaaaattaaccccgatttaaagagttctccgcgcgcaatgcttcagcattacaatgaatgtttggaaaatctagacaatccagtcacggacgaacatcatttgttaacatttggaaaagaagttgctacaaaaatatttatcgaagcgtttgaatacagttacaccaacactaatgccatcagcatggacaaaacagatgaatttgattttattaaaccggcattgaaacctttgccagatgcaagaccgccatcgcttttggccaacgtgatgaacgaacgtaaaagaaaattacaaaacaccaactcaacggcaaaatgtttgctaccagcaccaccgccacaattgcgtaaacttgaaaaaaagaatcatttattgcctttgttttctttgtaattatattgttgcatttctatttctaatatcatagttttctaataaagtagtttcatatttttgtttttgtacagtaattgtttcttggtttaacaagatcacaaccaataacataaagaataacacaatcataacaaaaattaaaaagccgcatactactagaacaaattctttaattagcgatcggtttctatttacaaattggccgagctgatcgccttcagtcggcgagttgtgggcttggatgatgtcgacgatattgttgccggcgcgaccgcctgtcgctctcgatataatgtcggccgccgtcggtttcatgatgtgcttaactacaaataatagttgtacttgacgggcgtcaccgtgatgccgctgctaaaacctccgtccgttaagacgcgttgcgttacaaaattaatgtttgtccgattagcgtagtcggaataatcaaacgtgttgggcggactaaaatcgggcatgttgatgggcacaatgccgctggagctgatagcaatgctgtcgttcttgcaaaacagccgaatttttttgtagggctctgctttattcggcgcagacgacaccatctggtcaaagttgttcaattttatgattacgttgggtaccaattgataggggaaaattattttctggaacattttgacaaagtccacaaccgtttggctatagtcgggaatgccgagcaaagactgcgcctgtttaatgtatttgagactggagcggtttactgtagcgcaattggatggcacgtcgcccttcataagccggcgcgttctctcccaattcaatttgttgtacaaattatcaatctcctcgtgcggcagattgattacatagcgcgcgggctgtttgcgatattgaaagatgcaaaaaatgcgtttcaacgacaatatcttcaccatggtggacgtttccagattgaaacataacaaaaagtcattgctttccaccaattctttaaaatgagacagcggaatttcacaagcgatcggtcgcaaattgctttttattggaggcggaacgctttgaccgttgcggttttttagtaacgcgctgcacgcagattgcatgtccgtttcgggatacgtaaactcgatgggacatttggggttttcatggtgaacgatcatagtgttgcaataaaacaagttgttggtcaggagcacgctaaaaacacgcgtttcgcccgcaccgatttcggtgatgggtaccaacgggttccagtagactatggtggcggacgctgttttttttggcgatcgactgtctatgttaacatcatgctcgtgcctgtacactagcacagaattgaattttggaaattgttttttgtcaatgtacaaccggtcgtcgtctgtgggcacgtacacgatcaagttttcgattaatttgttgcctacgtcgctttgcggttccaccaaattgtgagggaacgcaaaaaagcgatcgctaatacaaacttgaatctgaaacgggcactccatcgtgatgtatatgtcttacttcattagactttagattattttaatttgtgaactcgtaccgtattcaatagggtgtcgggcacgtaattgtaatggtaaaacagatcctgttgaacacgtgcgttgttcactacgattgaaatgcaaaaatacatcaagtacataaacactatgattagaaaggtagcagacagaaaatatttcatctttaaatcttatgctagttgaataaaatacatagtacttttatacgtttatttatatttgttttctttgttataaccgtaattgtaaaacttgtgatcgtgctcgccaggcataatttctttgcacatcagcttgcgaatatatgtgacatcttcgtacaccgatttcttgatgttaccatcgtgaagcgttgtcggcttgagaggtttgcggtcgttgttgtaaaaattttgcaccgaataattatccatagtgcagcacaggcaatgtcactgatgcatatgctttaattttttattgcattcagttattatatgatttaataaacgtacacaatagcacgtttatcggttaaagataactttcaatatataaaagtgtttgaattgcgagaccgtcaacataacgtttatcaacgcgatgactaaacgacaatttgctttgctgtttgtgtggcaccacgacaaccaatttgtttgcaacacggacgaatacccgttttggcacaacattgaataccatgcacggcgctataaatgcatcgttttgtactgtgtggaaaacgacggatcgctacaactgcccgtttgcaaaaacataaatctcataaattataaaaaagcgtatcctcattattatggaaactgtgttgacagtatagtgaaacgtgctggcaaaattgattatatgaaagtaactgcaatgttaaacccccacctgttggacgtcgcgtacaattatttgctgttgatggacatggattgtgtggtgcaaagcgtgcaatggaaacaattgtcaaccgacacgtattgttttgagccgttttacgactctcaaattaaatggttgtacgcgcccaaaagcggacaaagttttgatagttatcttgaaaactatgcaactctaattcgagtcaaacaagtgcagcaacatcgaaaagaattaatactgcattgtgtggattttcttacaatgaaagcaaatgacaattttatggtgttcaaaaattatattaacatgattataaaagtgtatttgcaattttacaattacagatttcccatcaattttgaggacaacacgatgaaaccttgtgtaaatttaacttttagacgtggcggcagttggaaaactcaactgcaacccgtatgcaattatgtttacaaaagtaaaaatatgccaaaatttattaaataaaacaaattaatttaaacaagcgtttttattgacaatactcacatttgatattatttataatcaagaaatgatgtcatttgttttcaaaattgaactggctttacgagtagaattttacttgtaaaacacaatcaagaaatgatgtcatttttgtacgtgattataaacatgtttaaacatggtacattgaacttaatttttgcaagttgataaacatgattaatgtacgactcatttgtttgtgcaagttgataaacgtgattaatatatgactcatatgtttgtgcaaaaatgatgtcatcgtacaaactcgctttacgagtagaattctacttgtaacgcatgatcaagggatgatgtcatttgtttttttaaaattcaactcgctttacgagtagaattctacttgtaaaacacaatcgagggatgatgtcatttgtagaatgatgtcatttgtttttcaaaaccgaactcgctttacgagtagaattctacttgtaacgcaagatcggtggatgatgtcattttaaaaatgatgtcatcgtacaaactcgctttacgagtagaattctacgtgtaaaacacgattacagcacttcgtagttgtatcgaaaattgttcaatggctctttgttaatgtcgtaattgattaatatgtcgtacaatttggcggcgttgtgtttgcacacgaccgtttttagttcttgaaacattttttcgtgtatgtttagcatgttgtatttcagagtgcgatgtgtaatgctggtgacgagcatcaaaatgataaaatctaaagcggctaatttgtaatcccgttcatacgctctgtaatcgccaacaactctgtggccagatctttttagattttgacaggcgttatggtacgaattgataatatttactatagtttctcttgttatcggtttgtcgattaaactgttaacaaacatcacgttgcccaagcgcgacggtttagacaccgacttgttttttgtctgttcaaatttgtacaaattaaaaacgctcatagactggtcgtcaggcagtgtgtcgttatacaaacaaaatggtaaaacgtttaattcgacaaacgacgagcacattaaagtttgttggctgttaacgtcctggggatgtaaactgttattcataacgtaacacacttcaatgtcggaatgcttgttttcaaatttgtccttgtctacagtttcaatggtgattgagcgaggtttgagtttattttgtaaattcatttggatattttcaatatggtataccaccgacacgttgtgagccagcgatccttgattggttttaatcatattcaaaatattcatgatatggttgaaaaaagagtctgtcaaaacgtttgtgtcgttgttaaatatcgctttccagggtttactgttgcgtgactcaacgacggccgtgtaacataacaagcgcgccagttgcatgtgcgacaacttaatgttatcaatgtcggtgatgtttggcaccagattttcattgccgtcttccagtagcgtgctcagttcggtcgagtagttattcaacgatcgattgtgcgattcaaacaagtttactatcgcaggttgtacatagttttttatgtcgtcaaattgaattatatcgatcttgtccttgttctccagcataaacgacaaattttttaggtcgaatttaatatttggcgcgttttcgttggactttttgtaatttaacaacatcgccaacagtttgtgtaactcgccgttagcttgatctttgctaaacagtttattggtagcgtaattcacgttgtcgttcaaaaacagcaactcgttgatgatcattttttgtaaaagcgcgtacttgctcatgttgacagaatctcttacatttcagttgtaaacgcgtctgtacaaattggccatgcgattcggaatgcacacggggatcgtgcgagccagtgccgtttggcgaaatagcattttttcatagccgctcgaacaatcgcacgcgtccggcgaaaattgcaccgtgttcaaattcatattcaaccggccgtcgttgcatagataaggcctcggtgttcccgtatcgtccaccaagtctctgtacgtgctcacgcatgtttgagacacgacaaaatctccgccggcggagaaaacgtgaaccaagcccagtgcgggatcgcattctatcaagtccggagcctgcgcgtttaccaaagcgtcggaggcgttgcaaaagccatcctggcaggtcaactcgtttgcagcgctggagatcacgcagttgtctctacactgctgatccgtcacgcacggtaaccggttcaatgaacaatctacgcctcgattgcgctgaaacgtaaaatttaacggcggcgcttccaactcgttaatgtgcatgtatgcatcttgcaaaataaatttttgaacaaatttaaacgtgtacatgtacacgattagtataattaccagtagaataagtatttgccaaaagttcaacatgatcgtcttaactgagtgtgaaaagcgtggtgtgacgcacgaaatgactggttgcgcaaaaaataaaccggggtctatataactcggcgtcgaccgcgttcatttttaccgtcatgcatctgacggctaatgtattgctcgttcctaacgcgctcaaaaagcgggacgtgaaatacatttataatacctatttgaaaaattacagtgtaattgaaggtgtgatgtgttgcaatggcgattgtttggccgtggtggtgttggaccgaaatcagctgcaaaacacggacatggaagtgttggagagtttagaatacactagtgacaacattgaactgttatgcgaaaaaatatgtgtgatagttgataattacgacaagtattaccaaaaaaattgtgtataaataaaataccaaattttattatatcattttgttttatttaataattaaagaatacaacgccacatctattcctagtacaacaaataatttgattattatttttgagtgcacattaaaaaataacaaacagtgtaaaaatactacagaataatacaatacataaatattatagtaaatagctgcaattttgatagcgtaatttatactttgatatttttcaacgtacaacgttaaatgttgatacgcattattcacaaataacaaaatttttctaatatgccatttgtccgcaattgtttttgcgatatcaaagcctttttcaaacaattgaaaaattgcaaacaaaaccacgtacatgacgttatacatagtgttaaagtttttacataacaattctataatgaagaaaattgctaaacacggcatgagcgcgcacataatcgcgttggccgcaaatatctcgtacgtacaaaaatactcggacattctccaataagtaaaatgcattttgctattatactgttgtttcttctagtgattattgcaatagtgtacacgtatgtagacttgatagatgtgcaccatgaagaggtgcgttatcctattacggtttttgacaacacacgcgcgccgcttattgaaccgccgtccgaaatagtaatcgaaggcaatgcacacgaatgtcacaaaactttgacgccgtgcttcacacacggcgattgcgatctgtgccgcgaaggattagccaactgccagttgtttgacgaagatacaatagtcaagatgcgtggagatgacggccaagaacacgagacgcttattcgagcgggagaagcgtactgcttggctttggatcgagaacgcgcccgatcgtgtaaccccaacacgggtgtgtggttgttggccgaaactgaaactggtttcgctcttttgtgcaactgcttacggcccggacttgttacgcagctcaacatgtacgaagactgcaacgtgcccgtgggctgcgcgcctcacggccgtatcgacaatatcaacagcgcttcgatccggtgcgtgtgcgacgacgggtacgtgagcgactataacgccgacaccgaaactccgtattgccgtccgcgcaccgtgcgcgacgtaatgtacgacgagagtttttttccgcgggcgccatgcgcagacggccaagttcgtctggatcatccggcgctcaatgatttttaccgcagacactttagactcgaagacatttgcgtgatcgacccttgctcggtggacccgattagcgggcaacgcacatcgggacgcttatttcaccaaccaaccgtaaatggtgtgggaatcaacggatgcaattgtccggccgatgacgggttactgcccgtgtttaatcgacacaccgccgacacgggcatggttagacaaagcgaccgcaccgtcgcgaacgcttgcttgcagccgtttaacgtgcacatgttatcgttgcgtcatgtggattacaaatttttctggggccgcagcgaccacaccgagtttgccgacgcggacatggtgtttcaagcgaatgtcaaccaactcagtcacgaacggtatcgagcgattttgtactcgttgctcgagtcgcacccggacgtaacagaaatcgtaacagtcaacatgggtgtcatgaaaatttccgtgtcatacgataccacattgaaaaatatactattaccatcttctgtttttaggctatttagatttaaagaaagtggcactgctcagccggtatgcttctttccaggcgtaggacggtgcataaccgtcaattccgattcgtgcatcaggcgacacgctggtggtcaagtgtggaccgcagaaacgttcaccaactcgtggtgtgtactgagtcgtgaaggtacgcatataaaagtttggagtcgcgcgtcacgatatccacgcggagacgcgcctgcagcgttaagattgcgcggcttctttctgaacaacgatcgcgaacgaaacacaataagagcggtcactacaggcgacatgacccaagggcaacaaatagacgcattaacccaaatacttgaaacttaccccaactactctgtataacaacatgagcattttaaaagttgtagaagcgtgcgatttggcacacacttttttgaaattgggttatttatttagggccaagacttgtttggatatcgctttagataatttggaactattgcgtcgaaagactaacataaaagaagtggcagtcatgttaaacaagaaaactacagagtgtttgcaattgaaacgaaaaatagataaaaaaattgcacaacgtgttttaataaaaatttacactatcaaatgatgacatcataacgggttcaatattctgtgtgcaaaaataaatgacatcatatttcaaacttgttttacgcgtaaaattctactggtaaaacaagtttgagatatgatgtcatcatcacaaataatagtatgtaataaaataaacatatttgtgtgtaaatataatttattacaaataaattttacattgaatcaatctgtcttcgtgtttgttgtaaggtcttcgaatcttgtgtttcagcccctcgggatggtcaaaatgcgccgtagtaattgttaatggatctttcaacgattttttgcccatggcgagtgtgacaaacgcggccacgacaaacagcaggataatcagtttcatggtgttctatatttgacaatatatgggtcgcttctaaatcaccttgtccccaaaagcctcttttatagttttttagaacacgttgtgtattccaacagtaattgttccatctctttcaacagccattcagcatccggtcgttgactgtaatcatgctgaattaatttacaaacaatttcggtcaatttaggatggccttgggataaacttgccggcatttgctgtacattgtttctaaagttagttagcgtagtttcgcgttccaaagcagtcttgaagggcattatcaattcgaataaaacaatgcccaaactatacatgtcatttttgggggtgtacacttttttgatttgttctggtgcagcgtacaaagttatattttgagggttgtttttgataaacgttttgtatagactgccaaacatgccgcccacatacaaatcaaagtcgggcccagtcatgaaaatatcttcgggattaatattgtggtgcacgatatttacggaatgaatcgctttcacggcgctcaccaaatcaacaaacttgctaatataaaagccaaaatccgccggaactttaatgttggtctttgcaaaagtttgcaaattgcgttgtttcaaatagtcgctcaacatgtactcgtttagaggcgacgcaaaatatatgcggtgctgccgcggattcaaataaaccaattgttcgggtttcatggtatacagttaagtgttaacgcgtcactaaattcagacacgagcgcacgccctatatacatacaatttatcgcacaagatgcttaacgcgatctgtttataaactaaaacgcactgcaataaattttagcaagcatttgtatttaatcaatcgaaccgtgcactgatataagaattaaaaatgggtttgtttgcgtgttgcacaaaatacacaaggctgtcgaccgacacaaaaatgaagtttccctatgttgcgttgtcgtacatcaacgtgacgctgtgcacctacaccgccatgttggtgggatacatggtaacattcaatgactccagcgaattgaaatatttacaatactggttgctgttgtcgtttttgatgtccgtggtgctaaacgctccgactctgtggacgatgctcaaaaccacagaagcccatgaagtaatttacgaaatgaagctgttccacgccatgtactttagtaacgtgctgttgaattatgtggtgtttttggacaatcaaatgggtacaaattttgtttttgttaacaatttaattcactgttgtgtactttttatgatatttgttgaattgcttatcctgttgggccacacaatgggcacgtacacggattatcaatatgtcaaatcgtgttatatggttatattgtttgtttcagttatgagtgttactattgttatgggtttagagtgtttgaaaacgaaactaattgataacagtttgatgtttaacgcgtttgtgtgcgctttgtacattgtgattgcaataatgtggtctttaaaaaataatttgactagttattacgtttcaaatttacaaagtattcaagttgttccgttttcatacaacgatccgccgccaccgttctctaacattgtaatggatgacataaaaaataaaaaataatttataaaaatgttttttattctttcacaattctgtaaattctaaacaaaaaatataaatacaaacttattatgttgtcgtctaaataaacatcaatttgtaaatctggacacctattcatatcattgatattacagtctactatacaacaattaaaactaaccaaattatctttacaacaattaaagcaattaaaacaatttaaataatcttcattgtcgtcgtataagtttatttgcactgtagacggtgttacacagcgatccattcgacgttcgtgttcgatcaactttctcgccaacttgtaccataaaaattgtttggacaaaaagttttccaacaatggtaacggccaattcaacgtgacgatgcgcacgtcctcgggtatgcatttgttaaaaaacacacagctcgctttaccaaacgaaagcaaaggtactaaatatggcgccattggctgatttgttattccaagataattacaaataaactgatccgtcgtggggtgataactggcaggtgtcagctttaaataatcttcaacgttgttgtcgcgcaaaagtctgcattttacacgcgttgttaatcccacgacttttgcatgtaaaatcggatccaaatactgcagaatcgtgtctataatttctaatggtaaacgtatgcgttttgctcgtgggcgctttgtaacgctcgacatcctaataacaactaacacaaaactaaaatgatactcaatatattgcttttacagttcatctttaggtttaaactgtgcgtttatcgcgttgagcaagtcgccgttatcggcatcaatctcccaagcaaacaggccgcccaatttatttcggtcgacatatttaacttttcctaacacagagtcgacgctgtcaaacgaaatcaaatcacctttacttttatcgaaaacgtacgacgcttgagcggcgctgtcaaacgtgtacacataattgttgagatctttttgaatttgacgataatctacaacaccgtcctcccacgtgcccgacaccggcccgttgccagtgccggaaaaatagttgtcattcgtataatttgttacgccggtccagccgcggccgtacatggcgacgcccacaattattttgttgggatcgacgccttgtttcagtaacgcatcgacagcgtagtgtgtagtgtatagctcttccgagttccaacttggcgcgtagactgttgtttggtagcccaaatccgtgtttgaccaagcccctttaaaatcgtaactcatgagaaatattttgcctaatgacttttgcgcttcggcgtagtttaccacggcaatcttgtcgtaacccgcgcttatagcgcttgttaattcgtaaaccctgccggtttgcgcttcgaggtcgtctagcattgcgcgcagctcctccaacaacaaaatgtatgttttggcgtcacgctccgcatcgcccaacgacgggttagcccctttgccgcccggaaactcccaatcgatgtctacaccgtcaaagaatttccacacttgcagaaattccttaaccgaatctacaaaaacgtttcttttttcaacatcgtgcataaaataaaatgggtctgatagagtccagcctcctattgaaggaagaatttttaaatgggggtttgctaattttgccgccatcaactgtccaaaattgcctttatacggctcgttccaagcggacacacctttttggggtttttgtacggcggcccacggatcgtgaatggcaactttgaaatcttcgcgtcccttgcacgatctttgcaaagattcaaagcttccgggtatcgttttgagggcgtcgtttattccatcgccgccgcagatgggtatgaaaccatacaacaagtgtgataaatttggcaagggaactttgtctacgggaaagttgcgcccgtacacaccccactcaacaaagtacgcagcgacaattttatcctctctcctgccaggtttgttgttttccagccatgtgtattcgagcggtgccagatggccgccgtcggtgtctgcgactttgaccaacacgggatcgctcacggaacagccgtcctcattgcaaagtttgacacgcatgttaaattgcccgctcacaagaactttaatggtagcccttttactttcggcgtcgcctttccatacctgctgctcgtcaaacaacacgtacgctatgtcgccaatgtcgccgttccagacgttccaactgacttgaacgtcgacttgttctttaggctttattaaattttcgtaagcggtggcctcgtaatttatttctacgagcgcataattgcgatcggcccaatcgatcaccggcgtgccgggaatcgcgttagaaacggcgaccaaccacaaaacgtttaacaatttgtacaacattttaatttatcttaattttaagttgtaattattttatgtaaaaaaatgaacaaaattttgttttatttgtttgtgtacggcgttgtaaacagcgcggcgtacgaccttttgaaagcgcctaattattttgaagaatttgttcatcgattcaacaaagattatggtagcgaagttgaaaaattgcgaagattcaaaattttccaacacaatttaaatgaaattattaataaaaaccaaaacgattcggccaaatatgaaataaacaaattctcggatttgtccaaagacgaaactatcgcaaaatacacaggtttgtctttgcctattcagactcaaaatttttgcaaagtaatagtcctagaccagccaccgggcaaagggccccttgaattcgactggcgtcgtctcaacaaagtcactagcgtaaaaaatcagggcatgtgtggcgcctgctgggcgtttgccactctggctagtttggaaagtcaatttgcaatcaaacataaccagttgattaatctgtcggagcagcaaatgatcgattgtgattttgtcgacgctggctgtaacggcggcttgttgcacacagcgttcgaagccatcattaaaatgggcggcgtacagctggaaagcgactatccatacgaagcagacaataacaattgccttatgaactccaataagtttctagttcaagtaaaagattgttatagatacattaccgtgtacgaggaaaaacttaaagatttgttacgccttgtcggccctattcctatggccatagacgctgccgacattgttaactataaacagggtattataaaatattgtttcaacagcggtctaaaccatgcggttcttttagtgggttatggtgttgaaaacaacattccatattggacctttaaaaacacttggggcacggattggggagaggacggatttttcagggtacaacaaaacataaacgcctgtggtatgagaaacgaacttgcgtctactgcagtcatttattaatctcaacacactcgctatttggaacataatcatatcgtctcagtagctcaaggtagagcgtagcgctctggatcgtatagatcttgctaaggttgtgagttcaagtctcgcctgagatattaaaaaactttgtaattttaaaaattttattttataatatacaattaaaaactatacaattttttattattacattaataatgatacaatttttattattacatttaatattgtctattacggtttctaatcatacagtacaaaaataaaatcacaattaatataattacaaagttaactacatgaccaaacatgaacgaagtcaatttagcggccaattcgccttcagccatggaagtgatgtcgctcagactggtgccgacgccgccaaacttggtgttctccatggtggttatgaggttgcttttttgttgggcaataaacgaccagccgctggcatctttccaactgtcgtgataggtcgtgttgccgatggtcgggatccaaaactcgacgtcgtcgtcaattgctagttccttgtagttgctaaaatctatgcattgcgacgagtccgtgttggccacccaacgcccttctttgtagatgctgttgttgtagcaattactggtgtgtgccggcggattggtgcacggcatcagcaaaaacgtgtcgtccgacaaaaatgttgaagaaacagagttgttcatgagattgccaatcaaacgctcgtccaccttggccacggagactatcaggtcgtgcagcatattgtttagcttgttgatgtgcgcatgcatcagctcaatgttcattttcagcaaatcgttttcgtacatcagctcctcttgaatatgcatcaggtcgcctttggtggcagtgtctccctctgtgtacttggctctaacgttgtggcgccaagtgggcggccgcttcttgactcggtgctcgactttgcgtttaatgcatctgttaaacttgcagttccacgtgtttttagaaagatcatatatatcattgtcaatcaaacagtgttcgcgtgtcaccgactcggggttatttttgtcatctttaatgagcagacacgcagcttttatttggcgcgtggtgaacgtagacttttgtttgagaatcatactcacgccgtctcgatgaagcacagtgtccacggtcacgttgatggggttgccctcagcgtccaaaatgtatacctggcactcgtccgtgtcgtcctggcactcgagcctgctgtacattttcgaagtggaaatgccgcatcgccacgatttgttgcacgtgtggtgcgcaaagtgattgttattctgccgcttcaccaactctttgcctttgacccactggccgcggccctcgttgtcgcgaaaacagtcgtcgctgtcactgccccaacggtcgatcagctcttcgcccacctcgcactgctgcctgatgctccacataagcaaatcctctttgcccacattcagcgttttcatggtttcttcgacgcgtgtgttgggatccagcgagccgccgttgtacgcatacgcctggtagtaccccttgtagccgataatcacgttttcgttgtagtccgtctccacgatggtgatttccacgtccttttgcagcgtttccttgggcggggtaatgtccaagtttttaatcttgtacggacccgtcttcatttgcgcgttgcagtgctccgccgcaaaggcagaatgcgccgccgccgccaaaagcacatataaaacaatagcgcttaccatcttgcttgtgtgttccttattgaagccttggtgtgactgatttactagtagcattgaggcatcttatatacccgaccgttatctggcctacgtgacacaaggcacgttgttagattaataatcttatctttttatcttaattgataagattatttttatctggctgttataaaaacgggatcatgaacacggacgctcagtcgacatcgaacacgcgcaacttcatgtactctcccgacagcagtctggaggtggtcatcattaccaattcggacggcgatcacgatggctatctggaactaaccgccgccgccaaagtcatgtcaccttttcttagcaacggcagttcggccgtgtggaccaacgcggcgccctcgcacaaattgattaaaaacaataaaaattatattcatgtgtttggtttatttaaatatctgtcaaattacaatttaaataataaaaagcgtcctaaagagtattacacccttaaatcgattattagcgacttgcttatgggcgctcaaggcaaagtatttgatccgctttgcgaagtaaaaacgcaactgtgtgcgattcaggagagtctcaacgaggctatttcgattttgaacgttcatagcaacgatgcggccgccaacccgcctgcgccagacattaacaagttgcaagaactgatacaagatttgcagtctgaatacaataaaaaaattacctttaccactgatacaattttggagaatttaaaaaatataaaggatttaatgtgcctgaataaataataataagggttttgtacgatttcaacaatgaacttttgggccacgtttagcatttgtctggtgggttatttggtgtacgcgggacacttgaataacgagctacaagaaataaaatcaatattagtggtcatgtacgaatctatggaaaagcatttttccaatgtggtagacgaaattgattctcttaaaacggacacgtttatgatgttgagcaacttgcaaaataacacgattcgaacgtgggacgcagttgtaaaaaatggcaaaaaaatatccaatctcgacgaaaaaattaacgtgttattaacaaaaaacggggtagttaacaacgtgctaaacgttcaataaacgcttatcactaagttaatatactaaaaatcacatagtcactacaatatttcaaaatatgaagccgacgaataacgttatgttcgacgacgcgtcggtcctttggatcgacacggactacatttatcaaaatttaaaaatgcctttgcaggcgtttcaacaacttttgttcaccattccatctaaacatagaaaaatgatcaacgatgcgggcggatcgtgtcataacacggtcaaatacatggtggacatttacggagcggccgttctggttttgcgaacgccttgctcgttcgccgaccagttgttgagcacatttattgcaaacaattatttgtgctacttttaccgtcgtcgccgatcacgatcacgctcacgatcacgctcgcgatcacgttctcctcattgcagacctcgttcgcgctctcctcattgcagacctcgttcgcgatctcggtcccggtctagatcgcggtcacgttcatcgtctcccaggcgagggcgtcgacaaatattcgacgcgctggaaaagattcgtcatcaaaacgacatgttgatgagcaacgtcaaccaaataaatctcaaccaaactaatcaatttttagaattgtccaacatgatgacgggcgtgcgcaatcaaaacgtgcagctcctcgcggcgttggaaaccgctaaagatgttattttgaccagattaaacacattgcttgccgagattacagactcgttacccgacttgacgtccatgttagataaattagctgaacaattgttggacgccatcaacacggtgcagcaaacgctgcgcaacgagttgaacaacaccaactctattttgaccaatttagcgtcaagcgtcacaaacatcaacggtacgctcaacaatttgctagccgctatcgaaaacttagtaggcggcggcggcggtggcaattttaacgaagccgacagacaaaaactggacctcgtgtacactttggttaacgaaatcaaaaatatactcacgggaacgctgacaaaaaaataagcatgtccgacaaaacaccaacaaaaaagggtggcagccatgccatgacgttgcgagagcgcggcgtaacaaaacccccaaaaaagtctgaaaagttgcagcaatacaagaaagccatcgctgccgagcaaacgctgcgcaccacagcagatgtttcttctttgcagaaccccggggagagtgccgtttttcaagagttggaaagattagagaatgcagttgtagtattagaaaatgaacaaaaacgattgtatcccatattagatacgcctcttgataattttattgtcgcattcgtgaatccgacgtatcccatggcctattttgtcaataccgattacaaattaaaactagaatgtgccagaatcagaagcgatttactttacaaaaacaaaaacgaagtcgctatcaacaggcctaagatatcgtcttttaaattgcaattgaacaacgtaattttagacactatagaaactattgaatacgatttacaaaataaagttctcacaattactgcacctgttcaagatcaagaactaagaaaatccattatttattttaatattttaaatagtgacagttgggaagtaccaaagtatatgaaaaaattgtttgatgaaatgcaattggaacctcccgtcattttaccattaggtctttagatttggtaaggctagcacgtcgacatcatgtttgcgtcgttgacctcagagcaaaagctgttattaaaaaaatataaatttaacaattatgtgaaaacgatcgagttgagtcaagcgcagttggctcattggcgttcaaacaaagatattcagccaaaacctttggatcgtgcagaaattttacgtgtcgaaaaggccaccaggggacaaagcaaaaatgagctgtggacgctattgcgtttggatcgcaacacagcgtctgcatcgtccaactcgtccggcaacatgttacaacgaccagcgcttttgtttggaaacgcgcaagaaagtcacgtcaaagaaaccaacggcatcatgttagaccacatgcgcgaaatcatagaaagtaaaattatgagcgcggtcgttgaaacggttttggattgcggcatgttctttagccccttgggtttgcacgccgcttcgcccgatgcgtatttttctctcgccgacggaacgtggatcccagtggaaataaaatgtccgtacaattaccgagacacgaccgtggagcagatgcgtgtcgagttggggaacggcaatcgcaagtatcgcgtgaaacacaccgcgctgttggttaacaagaaaggcacgccccagttcgaaatggtcaaaacggatgcgcattacaagcaaatgcaacggcagatgtatgtgatgaacgcgcctatgggcttttacgtggtcaaattcaaacaaaatttggtggtggtttctgtgccgcgcgacgaaacgttctgcaacaaagaactgtctacggaaaacaacgcgtacgtggcgtttgccgtggaaaactccaactgcgcgcgctaccaatgcgccgacaagcgacggctttcattcaaaacgcacagctgcaatcacaactatagtggtcaagaaatcgatgctatggtcgatcgcggaatatatttagattatggacatttaaaatgtgcgtactgtgattttagctcagacagtcgggaaacgtgcgattctgttttaaaacgcgagcacaccaactgcaaaagttttaacttgaaacataaaaactttgacaatcctacatactttgattatgttaaaagattgcaaagtttgctaaagagtcaccactttagaaacgacgctaaaacacttgcctattttggttactatttaactcatacaggaaccctgaagaccttttgctgcggatcgcaaaactcgtcgcccaccaaacacgatcatttaaacgactgtgtatattatttggaaataaaataaacctttatattatatataattcttttatttatacatttgtttatacaattttatttacgacaaatattgactcgttgttcagaaagtttaataagcttgtcaatttcttcggcttgcaaagggctgccaacgcgttcgttttgaatgcgcgtaatccggtttacggtattgttggcgcgaacaataaactcctcaactggcaaattaacaattttgtttgcgtactcattgtgcactgcggccaggttttgtagaatgttttcgggaaaaatggcaattctattaaatttgacatgtttttgattgtatacatagttttgatattcttccagcgtaggatatttgtttaaactcttgacgcattcaatgtacaatttgtgcagtgacaaaattctgttaaaatccaaacgagaacatttctcaaaagttatttcttgaccgttgaaatgtacactttgcaattgtttcaataaactgtcgtaaaaagtttttccttcttcaagcacaaacgcggggcgcatcgtgttatctacaacgcttatgtacttgtcaaaatcttcaattatatgatagaaatacaaatatctctccgcgtttatggacgtgtcgtttaaaacatgttcgtcaacaactccgttatgatttactttcaaaaatttcaaatcttgcaaagcgtccgcgttggtcaacttgttgataataaatttgtctttgcattcaaacgctctgtttgcaatccactccacagcgtccaaaacggacatgcgtttaaacatgttgatacgttttagacaatacgctcgtttttttaccgcctcaacgttcacgtccgtgtagtcgcaccattgcaggatttgcaacatgtcctcggcaaaatgcgcgaactgccgcagcttttcctttccaaaatgttgattgtcgtgtttaaaaagcaacgttgaaatttccgagacataccacaaagccgtgggcaattttactttgatcagcggctccatagccaggttgctgaacccgatcatgcattccgtgttgttaatgcggtaaatgacatagcgtttaaagtagtcctttacattatcgtcaatgtattctgcgtcgtttatgtgcttgtacagcaaatagtacataaggcccgcgttaaacgcgacctttttagcgtcaaaatacgtgcacgccaacacgtaatcgttgtattcgtcgaattgctcgttgggcactatggcgcccgtaaaagggcgtctgctgcgcggtgacaaacgcgttccatgctgaatcaactgcttcaaactttccaaattataacaatattcaattgaatttttaatctctttattttggctccataaaagaggaaactcgagtcggcttttaaacttggtcaaactgccctgaattgtttcaaacaagttgtaatgtgttaacaatatggccggcacaccgctatcgttggctaaaatacaatcggggaatcgaatattttctacgttgctgtaatcgtacgcttcgtcgtcgtcgttggcaacaacatcgtcggtttcggcgtccacgctcgctaacttgttctgatagtgtaaatttttcattacatcaaaagcgtatgacttgttgcgattgtgcaaataatttatggccgtgctaatggtgctgtcgataattttatcaaaattgagaacatcggcgttatacaacgttttataaaattctgttgacttgaacgtgtttacaaactcatttttatttttaatctggtcaaaattcatactagaattgttagtttgtttgatttcgctgaatagccgctggcggagacgcttcagcttgtccacctcgtttaacacgttggcgtccgtcggcatggaattgataaatttgaaccgaacaaaagacagcagttcatcttttttcgatataaaattttcggttgtaatgatatcgtagttaaattctttggttaaattgacccattcgaccatttcatcgttgcgataaatcttgcagtccgagttgttgacaaacgccgaggcaacggacaaatcaatctgttccgtgttattattgatggcataaaacacaatgcgttcgaaactaaacggtttttcgtttagcaaatttttgcaaacgtttgcctcatttttggaaatttggccgtcggtcaccatgtacaaaagtttcaacttgccgtcgagcaagtttatattcttgtgaatccactttatgaattcgctgggcctggtgtcagtaccctcgccattgcggcgcaaataacgactcttgacgtctccgatttctttttggcggcaataagcactccaatgcaaatacaaaactttgtcgcaactactgatgttttcgatttcattctgaaattgttctaaagtttgtaacgcgttcttgttaaagtaatagtccgagtttgtcgacaaggaatcgtcggtggcgtacacgtagtagttaatcatcttgttgattgatatttaattttggcgacggatttttatatacacgagcggagcggtcacgttctgtaacatgagtgatcgtgtgtgtgttatctctggcagcgcgatagtggtcgcgaaaattacacgcgcgtcgtaacgtgaacgtttatattataaatattcaacgttgcttgtattaagtgagcatttgagctttaccattgcaaaatgtgtgtaatttttccggtagaaatcgacgtgtcccagacgattattcgagattgtcaggtggacaaacaaaccagagagttggtgtacattaacaagattatgaacacgcaattgacaaaacccgttctcatgatgtttaacatttcgggtcctatacgaagcgttacgcgcaagaacaacaatttgcgcgacagaataaaatcaaaagtcgatgaacaatttgatcaactagaacgcgattacagcgatcaaatggatggattccacgatagcatcaagtattttaaagatgaacactattcggtaagttgccaaaatggcagcgtgttgaaaagcaagtttgctaaaattttaaagagtcatgattataccgataaaaagtctattgaagcttacgagaaatactgtttgcccaaattggtcgacgaacgcaacgactactacgtggcggtatgcgtgttgaagccgggatttgagaacggcagcaaccaagtgctatctttcgagtacaacccgattggtaacaaagttattgtgccgtttgctcacgaaattaacgacacgggactttacgagtacgacgtcgtagcttacgtggacagtgtgcagtttgatggcgaacaatttgaagagtttgtgcagagtttaatattgccgtcgtcgttcaaaaattcggaaaaggttttatattacaacgaagcgtcgaaaaacaaaagcatgatctacaaggctttagagtttactacagaatcgagctggggcaaatccgaaaagtataattggaaaattttttgtaacggttttatttatgataaaaaatcaaaagtgttgtatgttaaattgcacaatgtaactagtgcactcaacaaaaatgtaatattaaacacaattaaataaatgttaaaatttattgcctaatattattttgtcattgcttgtcatttattaatttggatgatgtcatttgtttttaaaattgaactggctttacgagtagaattctacgcgtaaaacacaatcaagtatgagtcataatctgatgtcatgttttgtacacggctcataaccgaactggctttacgagtagaattctacttgtaatgcacgatcagtggatgatgtcatttgtttttcaaatcgagatgatgtcatgttttgcacacggctcataaactcgctttacgagtagaattctacgtgtaacgcacgatcgattgatgagtcatttgttttgcaatatgatatcatacaatatgactcatttgtttttcaaaaccgaacttgatttacgggtagaattctacttgtaaagcacaatcaaaaagatgatgtcatttgtttttcaaaactgaactcgctttacgagtagaattctacgtgtaaaacacaatcaagaaatgatgtcatttgttataaaaataaaagctgatgtcatgttttgcacatggctcataactaaactcgctttacgggtagaattctacgcgtaaaacatgattgataattaaataattcatttgcaagctatacgttaaatcaaacggacgttatggaattgtataatattaaatatgcaattgatccaacaaataaaattgtaatagagcaagtcgacaatgtggacgcgtttgtgcatattttagaaccgggtcaagaagtgttcgacgaaacgctaagccagtaccaccaatttcctggcgtcgttagttcgattattttcccgcaactcgtgttaaacacaataattagcgttttgagcgaagacggcagtttgctcacgttgaaactcgaaaacacttgttttaattttcacgtgtgcaataaacgctttgtgtttggcaatttgccagcggcggtcgtgaataatgaaacgaagcaaaaactgcgcattggagctccaatttttgccggcaaaaagctggtttcggtcgtgacggcgtttcatcgtgttggcgaaaacgaatggctgttaccggtgacgggaattcgagaggcgtcccagctgtcgggacatatgaaggtgctgaacggcgtccgtgttgaaaaatggcgacccaacatgtccgtctacgggactgtgcaattgccgtacgataaaattaaacagcatgcgctcgagcaagaaaataaaacgccaaacgcgttggagtcttgtgtgctattttacaaagattcagaaatacgcatcacttacaacaagggggactatgaaattatgcatttgaggatgccgggacctttaattcaacccaacacaatatattatagttaaataagaattattatcaaatcatttgtatattaattaaaatactatactgtaaattacattttatttacaatcatgtcaaagcctaacgttttgacgcaaattttagacgccgttacggaaactaacacaaaggttgacagtgttcaaactcagttaaacgggctggaagaatcattccagcttttggacggtttgcccgctcaattgaccgatcttaacactaagatctcagaaattcaatccatattgaccggcgacattgttccggatcttccagactcactaaagcctaagctgaaaagccaagcttttgaactcgattcagacgctcgtcgtggtaaacgcagttccaagtaaatgaatcgtttttaaaataacaaatcaattgttttataatattcgtacgattctttgattatgtaataaaatgtgatcattaggaagattacgaaaaatataaaaaatatgagttctgtgtgtataacaaatgctgtaaacgccacaattgtgtttgttgcaaataaacccatgattatttgattaaaattgttgttttctttgttcatagacaatagtgtgttttgcctaaacgtatactgcataaactccatgcgagtgtatagcgagctagtggctaacgcttgccccaccaaagtagattcgtcaaaatcctcaatttcatcaccctcctccaagtttaacatttggccgtcggaattaacttctaaagatgccacataatctaataaatgaaatagagattcaaacgtggcgtcatcgtccgtttcgaccatttccgaaaagaactcgggcataaactctatgatttctctggacgtggtgttgtcgaaactctcaaagtacgcagtcaggaacgtgcgcgacatgtcgtcgggaaactcgcgcggaaacatgttgttgtaaccgaacgggtcccatagcgccaaaaccaaatctgccagcgtcaatagaatgagcacgatgccgacaatggagctggcttggatagcgattcgagttaacgctttggcagtcacggtcagcgttttgatggcgatcacgttgagcgagtgcactaacgcggctttgtaagtctctcccaacatgcgcacggtcacgcgccgagtcgtgctaagcaacatgtgtttcatggccggaatgagagaagtgttaatttttttcaacatgcttttaaacccggacattagcatatcaaagccaatgtccgtagcaataccgaaaacgagcgcgtaatcttccaaaaacgatgttataattgactccaagtcttggtcgctgattgaacggtcgagcgcctcgaaatgttcgacacgtgcacgttcgttaccgcggtaattgtatgcgatcggagttttagtaaagccggtttcggccgtgtacgtgatctggacgggcgacccgttgacgatcatgcccaaatcgtttagtgttggatttttgttaaaaagtttttcaaattccaagtctgtggcgttatcgcgcacgctgcgccattgcgctagtattgcgttggagtccacgttgggtcgtggcggtagtatgctggaaggcgctttgtaatcaaaatcgcgcagttcgctaaaaatgttgttggccagcattttgaaagtgacaaagatcgtgtcgcccagcacgaatccgatgagcgattcccaccatctaaacgaacaaccgccgttgaatagctctctgccgaaacgtcgacagtaggcttcgttgaattcgcctttaaagcgttcgggaaacaaggggtcgggatcgggccgaacgttaaaagccggcacatcgtccacgcccatgatcgtgtgttcttcggtgcgcaagtatgggctgttaaagtacattttggacagcgagtccactaagatgcatttgttgtcgagcgtgtatctaaactcggcagactgaacttgggtttcggcgccttcacgcatggccgccgccctgtccaggtggtagcacgcgggctgcgcgtaacccacgctagtctcggaggtctgcgtgtacatgaacggcgtcgtgttggacacgacgccggtttcgtgaaacggatagcagctcatgctttcacacccgcgcttgctgaaagccagtttgacggccagcgctttgtcggccaatttcggcggcacataataatcgtcgtcacttgacgcgggacgcagcgtgtagtcgattagtatatgcggaaacctggtgcgccatctcgaaataaactcgagacgatgcatatgtatggcatacctactggcattagttaaatcgacggctgttaaaaccgccatgttatataggacttaaaataaacaacaatatataatgaaatatttattagattatattatagcaatacatttacatttattataacaatactttttatttaatctgattatattataacgatacatttttatttagacattgttatttacaatattaattaactttttatacatttttaaatcataatatataatcatttcgttgtgcatttcaaagcttttgatagcttcaaagtaatacatgaatttagagtattcaggaaaatgataaacgttggtaaacccgcatttggtacaatataacacgggatttttataatacagtttagtttttttacacaatttgcaatagttgttagttgtaggtttcaaaggaaacgtgattgcgccgtccaatacctgggtaaactttttgactttaacagtggcaaacacggttcctttgatacccgaaaatcggttgtcttgcagagcggccatcatttcgcttggctcttgaagtataaaacagttgacgtcatccaccacgtcgggtctggtgcacatgcttcggtagcgctgcaacactatattggtgtatgtttccctgagaacgagaccgccggtggtgctaagatcgattgtttgaatgcgctcgttgggctctttgtgatttcgaattatgcgccgaattatttcaaacactttgcagttgtgatcgtcaattctcaattctttaacttccgtcgtgtgctctaaacttacagggaaaatgtattggtaaaaaaacctctctctggctaaatagctgaggtcgaccaaattgatagaaggatatatttcgtacgaggtttttggaacgttgtgatatagatagcatttttgacagcagatgtctatgcggtcaggatcgtccaacggcttttcgatgtgaaccacaacatacaaaaaccattcgcgcgtgttgtctttgaatctataattgcaagtggtgcatcgcgaatcgctcatgtgctccatagtcttcttgtatttcacaggcctgcttgcaaatttgcccgtcatgcgcatatctttgctgtttatgtagcccataatgtaattggtggaaaattttagcgtggctttcatgatgtcgcgttctaaatcgctcatgaaatgcatacgtagatcgcgctcttgtttgaaatccagtttgtcgctgtacgcgggcaaaccttcaaacttgttcccaaactcgggcggcacaaaatatccatcttttctgttgacgactggttttttacttacaatgctgctgtgctccaacggcttggccggagaggtgcgcgtaggctgtttaggcggagagatgcgcgtaggtggtttgatgttagattttggcggcggacgaacaggcgacggcggcgagttggcggcaggcgctggcaaagatttggcacgacccttgcccccggtccttggcgcgtcaaaaatgttattctctcgaaaaaaacggttcattgtaactgttagttagcactcagaaatcaacacgatactgtgcacgttcagccatcgagaggctttatatatggaaaccttatctatagagataagattgtatatgcgtaggagagcctggtcacgtaggcactttgcgcacggcactagggctgtggaggggacaggctatataaagcccgtttgcccaactcgtaaatcagtatcaattgtgctccggcgcacacgctcgcttgcgcgccggatagtataagtaattgataacgggcaacgcaacatgataagaaccagcagtcacgtgctgaacgtccaggaaaatataatgacgtcaaactgtgcgtcatcgccatattcgtgcgaggcaacgtccgcttgcgcagaagctcaacaggtaatgatcgataactttgttttctttcacatgtacaacgccgacatacaaattgacgcaaagctgcaatgcggcgtgcgctcggccgcgtttgcaatgatcgacgataaacatttggaaatgtacaagcatagaatagagaataaatttttttattactatgatcaatgtgccgacattgccaaacccgaccgtctgcccgatgacgacggcgcgtgctgtcaccattttatttttgatgcccaacgtattattcaatgtattaaagagattgaaagcgcgtacggcgtgcgtgatcgcggcaatgtaatagtgttttatccgtacttgaaacagttgcgagacgcgttgaagctaattaaaaactcttttgcgtgttgttttaaaattataaattctatgcaaatgtacgtgaacgagttaatatcaaattgcctgttgtttattgaaaagctggaaactattaataaaactgttaaagttatgaatttgtttgtagacaatttggttttgtacgaatgcaatgtttgtaaagaaatatctacggatgaaagatttttaaagccaaaagaatgttgcgaatacgctatatgcaacgcgtgctgcgttaacatgtggaagacggccaccacgcacgcaaaatgtccagcgtgcaggacatcgtataaataagcacgcaacgcaaaatgagtggtggcggcaacttgttgactctggaaagagatcattttaaatatttatttttgaccagctattttgatttaaaagataatgaacatgttccttcagagcctatggcatttattcgcaattacttgaattgcacgtttgatttgctagacgatgccgtgctcatgaactatttcaattacttgcaaagcatgcaattgaaacatttggtgggcagcacgtcgacaaacattttcaagtttgtaaagccacaatttagatttgtgtgcgatcgcacaactgtggacattttagaatttgacacgcgcatgtacataaaacccggcacgcccgtgtacgccacgaacctgttcacgtccaatccccgcaagatgatggctttcctgtacgctgaatttggcaaggtgtttaaaaataaaatattcgtaaacatcaacaactacggctgcgtgttggcgggcagtgccggtttcttgttcgacgatgcgtacgtggattggaatggtgtgcgaatgtgtgcggcgccgcgattagataacaacatgcatccgttccgactgtatctactgggcgaggacatggctaagcactttgtcgataataatatactaccgccgcacccttctaacgcaaagactcgcaaaatcaacaattcaatgtttatgctgaaaaacttttacaaaggtctgccgctgttcaaatcaaagtacacggtggtgaacagcactaaaatcgtgacccgaaaacccaacgatatatttaatgagatagataaagaattaaatggcaactgtccgtttatcaagtttattcagcgcgactacatattcgacgcccagtttccgccagatttgcttgatttgctaaacgaatacatgaccaaaagctcgatcatgaaaataattaccaagtttgtgattgaagaaaaccccgctatgagcggtgaaatgtctcgcgagattattcttgatcgctactcagtagacaattatcgcaagctgtacataaaaatggaaataaccaaccagtttcctgtcatgtacgatcatgaatcgtcgtacatttttgtgagcaaagactttttgcaattgaaaggcactatgaacgcgttctacgcgcccaagcagcgtatattaagtattttggcggtgaatcgtttgtttggcgccacggaaacgatcgactttcatcccaacctgctcgtgtaccggcagagttcgccgccggtccgtttgacgggcgacgtgtatgttgttgataagaacgaaaaagtttttttggtcaaacacgtgttctcaaacacggtgcctgcatatcttttaataagaggtgattacgaaagttcgtctgacttgaaatcccttcgcgatttgaatccgtgggttcagaacacgcttctcaaattattaatccccgactcggtacaataatatgatttacactgatcccactactggcgctacgactagcacagacgcgccgtccacaaactatttaaacaggctaactccaaacatgttcttgaccatcttggctgtagtagtaattattgctttaataattatatttgttcaatctagcagtaatggaaacagctcggggggtaatgtacctccaaacgccctggggggttttgtaaatcctttaaacgctaccatgcgagctaatccctttatgaacacgcctcaaaggcaaatgttgtagataagtgtataaaaaatgaaacgtatcaaatgcaacaaagttcgaacggtcaccgagattgtaaacagcgatgaaaaaatccaaaagacctacgaattggctgaatttgatttaaaaaatctaagcagtttagaaagctatgaaactctaaaaattaaattggcgctcagcaaatacatggctatgctcagcaccctggaaatgactcaaccgctgttggaaatatttagaaacaaagcagacactcggcagattgccgccgtggtgtttagcacattagcttttatacacaatagattccatccccttgttactaattttactaacaaaatggagtttgtggtcactgaaaccaacgacacaagcattcccggagaacccattttgtttacggaaaacgaaggtgtgctgctgtgttccgtggacagaccgtctatcgttaaaatgctaagccgcgagtttgacaccgaggctttagtaaactttgaaaacgacaactgcaacgtgcggatagccaagacgtttggcgcctctaagcgcaaaaacacgactcgcagcgatgattacgagtcaaataaacaacccaattacgatatggatttgagcgattttagcataactgaggttgaagccactcaatatttaactctgttgctgaccgtcgaacatgcctatttacattattatatttttaaaaattacggggtgtttgaatattgcaaatcgctaacggaccattcgctttttaccaacaaattgcgatcgacaatgagcacaaaaacgtctaatttactgttaagcaaattcaaatttaccattgaagattttgacaaaataaactcaaattctgtaacatcagggtttaatatatataattttaataaataattaaataatatacaatgtttttattaattatatttttaatattaattaaagtattaatatttaaaaaaatgaatcaaattcatctaaagtgtcacagcgataaaatttgtcctaaagggtattttggcctcaacgccgatccctatgattgcacggcgtattatctgtgtccgcataaagtgcaaatgttttgcgaattaaatcacgaatttgacttggactccgccagctgcaagcctatcgtgtacgatcacacgggcagcgggtgtacggctcgcatgtatagaaacttgttactatgaagagcgggtttccagttgcacaacactattatcgatttgcagttcgggacataaatgtttaaatatatcgatgtctttgtgatgcgcgcgacatttttgtaggttattgataaaatgaacggatacgttgcccgacattatcattaaatccttggcgtagaatttgtcgggtccattgtccgtgtgcgctagcatgcccgtaacggacctcgtacttttggcttcaaaggttttgcgcacagacaaaatgtgccacacttgcagctctgcatgtgtgcgcgttaccacaaatcccaacggcgcagtgtacttgttgtatgcaaataaatctcgataaaggcgcggcgcgcgaatgcagctgatcacgtacgctcctcgtgttccgttcaaggacggtgttatcgacctcagattaatgtttatcggccgactgttttcgtatccgctcaccaaacgcgtttttgcattaacattgtatgtcggcggatgttctatatctaatttgaataaataaacgataaccgcgttggttttagagggcataataaaagaaatattgttatcgtgttcgccattagggcagtataaattgacgttcatgttggatattgtttcagttgcaagttgacactggcggcgacaagatcgtgaacaaccaagtgactatgacgcaaattaattttaacgcgtcgtacaccagcgcttcgacgccgtcccgagcgtcgttcgacaacagctattcagagttttgtgataaacaacccaacgactatttaagttattataaccatcccaccccggatggagccgacacggtgatatctgacagcgagactgcggcagcttcaaactttttggcaagcgtcaactcgttaactgataatgatttagtggaatgtttgctcaagaccactgataatctcgaagaagcagttagttctgcttattattcggaatcccttgagcagcctgttgtggagcaaccatcgcccagttctgcttatcatgcggaatcttttgagcattctgctggtgtgaaccaaccatcggcaactggaactaaacggaagctggacgaatacttggacaattcacaaggtgtggtgggccagtttaacaaaattaaattgaggcctaaatacaagaaaagcacaattcaaagctgtgcaacccttgaacagacaattaatcacaacacgaacatttgcacggtcgcttcaactcaagaaattacgcattattttactaatgattttgcgccgtatttaatgcgtttcgacgacaacgactacaattccaacaggttctccgaccatatgtccgaaactggttattacatgtttgtggttaaaaaaagtgaagtgaagccgtttgaaattatatttgccaagtacgtgagcaatgtggtttacgaatatacaaacaattattacatggtagataatcgcgtgtttgtggtaacttttgataaaattaggtttatgatttcgtacaatttggttaaagaaaccggcatagaaattcctcattctcaagatgtgtgcaacgacgagacggctgcacaaaattgtaaaaaatgccatttcgtcgatgtgcaccacacgtttaaagctgctctgacttcatattttaatttagatatgtattacgcgcaaaccacatttgtgactttgttacaatcgttgggcgaaagaaaatgtgggtttcttttgagcaagttgtacgaaatgtatcaagataaaaatttatttactttgcctattatgcttagtcgtaaagagagtaatgaaattgagactgcatctaataatttctttgtatcgccgtatgtgagtcaaatattaaagtattcggaaagtgtgcagtttcccgacaatcccccaaacaaatatgtggtggacaatttaaatttaattgttaacaaaaaaagtacgctcacgtacaaatacagcagcgtcgctaatcttttgtttaataattataaatatcatgacaatattgcgagtaataataacgcagaaaatttaaaaaaggttaagaaggaggacggcagcatgcacattgtcgaacagtatttgactcagaatgtagataatgtaaagggtcacaattttatagtattgtctttcaaaaacgaggagcgattgactatagctaagaaaaacaaagagttttattggatttctggcgaaattaaagatgtagacgttagtcaagtaattcaaaaatataatagatttaagcatcacatgtttgtaatcggtaaagtgaaccgaagagagagcactacattgcacaataatttgttaaaattgttagctttaatattacagggtctggttccgttgtccgacgctataacgtttgcggaacaaaaactaaattgtaaatataaaaaattcgaatttaattaattatacatatattttgaatttaattaattatacatatattttatattatttttgtcttttattatcgaggggccgttgttggtgtggggttttgcatagaaataacaatgggagttggcgacgttgctgcgccaacaccacctcctcctcctcctttcatcatgtatctgtagataaaataaaatattaaacctaaaaacaagaccgcgcctatcaacaaaatgataggcattaacttgccgctgacgctgtcactaacgttggacgatttgccgactaaaccttcatcgcccagtaaccaatctagacccaagtcgccaactaaatcaccaaacgagtaaggttcgatgcacatgagtgtttggcccgcaggaagatcgctaatatctacgtattgaggcgaatctgggtcggcggacggatcgctgccgcgacaaactgttttttctacttcatagttgaatccttggcacatgttggttagttcgggcggattgttaggcaacaaggggtcgaatgggcaaatggtaacatccgactgatttagattggggtcttgacgacaagtgcgctgcaataacaagcaggcctcggcgatttctccggcgtctttaccttgcacataataacttccgccggtgttattgatggcgttgattatatcttgtactagtgtggcggcgctaaacaagaaatagccgccggtggccaagagtatgcccgttcctcctacttttaagctttgcatgtaactatgtagacgggggttttgctgcagtgcgttttgaacaccttcgggcgtgcgcacgttggtttccgggaagttttgtttgactgcattggatcgcgtctgcttggtgtggtaattaaagtctggcacgttgtccacgcgccgcaattggctcaatgagtttatttgagggtctgaaatgccctgaaatactccgcgtatgttggggacatcattgttacgagtaattctgtttatgtctgaagtgctcacaaactggttgttagatagttgatagcccggctgaaatctgttgtttccaatgttgcgtacactgggcgcgttgagcacatttgtgaaaccggcgggagtgcttgttaaaagacgcgtattatcagtaataaaactggcctgattaggatacaatttattgactgcgcgaagatttgaaaaaaaactcattttaaagcaaacttatttaataaatatatcacagtaaaggttttgcaaaactgccgtcgtcaatacaacacggcagcggcgtcatgttggtaaaatctaatcttctccttgctttagattctgggcgagaaggcgcatttgttgtgtaagttatttcgacgtctgcattatttgttgtgtaaggtatctcgacgtatgaagcaactttaacattgttataattttttttaaatattgatgcgctccacggcgcgcgttgatacggatgatatctctccattgtatgatcgctaaatttatataccgtttcaataaatatgttaaaacccaacatgttaattataatattcataatagtttgtttgttttcaataattatttttactgttttgaaatctaaaagaggtgacgatgacgaatcagacgacgggttcagttgctataacaaaccaattggagtaaattttccgcatcctactagatgtgacgctttctacatgtgtgtcggtttaaatcaaaaattagagttaatctgccctgaaggatttgaatttgatccagatgttaaaaattgtgttcctatatcagattatggatgtaccgctaaccaaaactaaaaataaaataaaatttatatagattaatgaaataaaatttatatagattaataaaataaaatttatttaatatattatactatttatattatttacaacacttaacgtctagacataacagtttgtaacttagaaactaaatcagagttactgcgctcaaactctgaaaatttggcttgagactcggccacctgcttacgcaattgttcttgcagattattcacagtcgattgcaactcttctgatttcttggtagattcttgcaagtcatagtttgccttttgtaaatctaattcggcgacagcatgcttgtgtttaagcataatgtagtcgctgtttaacatggtcattttatgttcaacttggctggtcttggctcgcagctcggacagttctttttgcaattgctccacatagttcaagtccgtggtgtgattgttgaccgtgttattttctaaaagctcgcgccaatgctgtttgatggaatcctggttacgagtgacgttaatgggcataaattctacatacccgtgcttattgtacacgcgacaatctgatgaagtagcgctgcaaaaacatttgtacacagaattgtccataattatcttgacataacacttgaaacacacagcatggttacaatgaatcgaagtcacaaacgaggaatttacgtttttagtgtctttaaaagtagtaaaacaaatattacacgaaacctctacttcttcttcgggttctgattgctgctgctgctgctgctgcggctgcggagactgcggcgaggcaaacaaatctggcgactgtggtattacgtaattcggcgaataagatggactataagtgggagaccttggggcaatctcattcatcagctgagcctcaagatctaaacctcgttgcagagccctctgcgcagctgtctccgacgcaatgttatcctggtactgctgggcagtgatgtcgggaaaccgttcacgatccacattttcactattaattagtatgacgtcatcctcttgacttaatagcggatcgtcattgctaatgttaacctgaccgtgcacgtaatacgtgacaccctgacgatggtaggtgcgcgtcaacggctcgttgacgttcccgataatctgcacgttttcttcgctgacacgctgctcctgacgccgctcctgacggcgatggctgcgactgcttgaagacggctggctgcgactgcttgaagacggctgggcttcgggagatgttgtaaagttgatgcggcgacggctgagagacagcctgtggcggcggctgctgctgggagtggcggcgttgatttggcgactcatggctgggctggtaggatactgttcactaggctgtgaggcttgaactgtgcttacgagtagaacggcagctgtatttatactgtttatcagtactgcacgactgataagacaatagtggtgggggaacttgccaggcaaaaatgaacttttttgtaatgcaaaaaagttgatagtgtagtagtatattgggagcgtatcgtacagtgtagactattctaataaaatagtctacgatttgtagagattgtactgtatatggagtgtcaggcaaaagtgaacttttttgcattgcaaaaaaattcattttaaatttatcatatcacaggctgcagtttctgttatctgtcccccactcaggcgtgcagctataaaagcaggcactcaccaactcgtaagcacagttcgttgtgaagtgaacacggagagcctgccaataagcaaaatgccaagggacaccaacaatcgccaccggtctacgccatatgaacgtcctacgcttgaagatctccgcagacagttgcaagacaatttggacagcataaacccccgagacagaatgcaagaagaacaagaagaaaacctgcgctatcaagtgcgtagaaggcagcgtcaaaaccagctccgctccatacaaatggaacagcagcgaatgatggcggaattaaacaacgagccggtgattaattttaaatttgagtgtagtgtgtgtttagaaacatattcccaacaatctaacgatacttgtccttttttgattccgactacgtgcgaccacggtttttgtttcaaatgcgtcatcaatctgcaaagcaacgcgatgaatattccgcattccactgtgtgctgtccattgtgcaatacccaggtaaaaatgtggcgttccttaaagcctaacgctgttgtgacgtgtaagttttacaagaaaactcaagaaagagttccgcccgtgcagcagtataaaaacattattaaagtgctacaagaacggagcgtgattagtgtcgaagacaacgacaataattgtgacataaatatggagaatcaggcaaagatagctgctttggaagctgaattggaagaagaaaaaaatcacagtgatcaagtagcttctgaaaaccgacagctgatagaagaaaatactcgtctcaatgaacagattcaagagttgcagcatcaggtgaggacattggtgccgcaacgtggcattacggttaatcagcaaattggccgtgacgacagtgcgccagccgagctgaacgagcgttttcgctcacttgtctattcgactatttcagagctgtttattgaaaatcgcgttcatagtattcaaaattatgtttatgccggaacttctgctgctagttcatgtgatgtaaatgttactgttaattttgggtttgaaaattaatgtgatatgaaatgtatatataaaaatgatggaataaataataaacatttttatactttttatgttttttttatttcatgtgattaagaaacttttaagatggatagtagtaattgtattaaaatagatgtaaaatacgatatgccgttacattatcaatgtgacaataacgcagataaagacgttgtaaatgcgtatgacactatcgatgttgaccccaacaaaagatttataattaatcataatcacgaacaacaacaagtcaatgaaacaaataaacaagttgtcgataaaacattcataaatgacacagcaacatacaattcttgcataataaaaatttaaatgacatcatatttgagaataacaaatgacattatccctcgattgtgttttacaagta

SEQ ID NO: 3 - protein, 296 aa, Porcine circovirus 3
VRRESPKHRWCFTINNWTPTEWESIVECGGSIARYLIIGKEVGKGGTPHLQGYVNFKNKRRLSSVKRLPGFGRAHLEPARGSHKEASEYCKKEGDYLEIGEDSSSGTRSDLQAAARILTETSGNLTEVAEKMPAVFIRYGRGLRDFCGVMGLGKPRDFKTEVYVFIGPPGCGKTREACADAAARELQLYFKPRGPWWDGYNGEGAVILDDFYGWVPFDELLRIGDRYPLRVPVKGGFVNFVAKVLYITSNVVPEEWYSSENIRGKLEALFRRFTKVVCWGEGGIKKDMETVYPINY

SEQ ID NO: 4 - protein, 214 aa, Porcine circovirus 3
MRHRAIFRRKPRPRRRRRHRRRYVRRKLFIRRPTAGTYYTKKYSTMNVISVGTPQNNKPWHANHFITRLNEWETAISFEYYKILKMKVTLSPVISPAQQTKTMFGHTAIDLDGAWTTNTWLQDDPYAESSTRKVMTSKKKHSRYFTPKPILAGTTSAHPGQSLFFFSRPTPWLNTYDPTVQWGALLWSIYVPEKTGMTDFYGTKEVWIRYKSVL

SEQ ID NO: 5 - DNA, 2000 bp, Porcine circovirus 3
  source 1..2000: mol_type = unassigned DNA
tagtattacccggcacctcggaacccggatccacggaggtctgtagggagaaaaagtggtatcccattatggatgctccgcaccgtgtgagtggatataccgggcagtggatgatgaagcggcctcgtgttttgatgccgcaggacggggactggataactgagtttttgtggtgctacgagtgtcctgaagataaggacttttattgtcatcctattctaggtccggagggaaagcccgaaacacaggtggtgttttacgataaacaactggaccccgaccgagtgggaatctattgtggagtgtggaggcagtatagcgagataccttattatcggcaaagaggttggaaaaggcggtaccccacacttgcaagggtacgtgaatttcaagaacaaaaggcgactcagctcggtgaagcgcttacccggatttggtcgggcccatctggagccggcgagggggagccacaaagaggccagcgagtattgcaagaaagagggggattacctcgagattggcgaagattcctcttcgggtaccagatcggatcttcaagcagcagctcggattctgacggagacgtcgggaaatctgactgaagttgcggagaagatgcctgcagtatttatacgctatgggcggggtttgcgtgatttttgcggggtgatggggttgggtaaaccgcgtgattttaaaactgaagtttatgtttttattggtcctccaggatgcgggaaaacgcgggaagcttgtgcggatgcggctgcgcgggaattgcagttgtatttcaagccacgggggccttggtgggatggttataatggggagggtgctgttattctggatgatttttatgggtgggttccatttgatgaattgctgagaattggggacaggtaccctctgagggttcctgttaagggtgggtttgttaattttgtggctaaggtattatatattactagtaatgttgtaccggaggagtggtattcctcggagaatattcgtggaaagttggaggccttgtttaggaggttcactaaggttgtttgttggggggagggggggataaagaaagacatggagacagtgtatccaataaactattgattttatttgcacttgtgtacaattattgcgttggggtgggggtatttattgggtgggtgggtgggcagccccctagccacggcttgtcgcccccaccgaagcatgtgggggatggggtccccacatgcgagggcgtttacctgtgcccgcacccgaagcgcagcgggagcgcgcgcgaggggacacggcttgtcgccaccggaggggtcagatttatatttattatcacttagagaacggacttgtaacgaatccaaacttctttggtgccgtagaagtctgtcattccagttttttccgggacataaatgctccaaagcagtgctccccattgaacggtggggtcatatgtgttgagccatggggtgggtctggagaaaaagaagaggctttgtcctgggtgagcgctggtagttcccgccagaattggtttgggggtgaagtaacggctgtgtttttttttagaagtcataactttacgagtggaactttccgcataagggtcgtcttggagccaagtgtttgtggtccaggcgccgtctagatctatggctgtgtgcccgaacatagtttttgtttgctgagctggagaaattacagggctgagtgtaactttcatctttagtatcttataatattcaaagctaattgcagtttcccattcgtttaggcgggtaatgaagtggttggcgtgccagggcttattattctgaggggttccaacggaaatgacgttcatggtggagtatttctttgtgtagtatgtgccagctgtgggcctcctaatgaatagttttcttctgacatagcgccttctgtggcgtcgtcgtctccttgggcggggttttcttctgaatatagctctgtgtctcattttggtgccgggc

SEQ ID NO: 6 - DNA, 645 bp, Porcine circovirus 3
  source 1..645: mol_type = unassigned DNA
atgcgccaccgtgctatcttcaggcgtaggcctaggcccagaaggaggaggagacaccgccgtcgttacgctagacgccgtctgttcatcaggagaccaaccgccggtacttactacaccaagaagtactccactatgaacgtgatcagcgtcggcaccccacagaacaacaagccttggcacgctaaccacttcatcactcgcctgaacgagtgggaaactgccatcaccttcgagtactacaagatcctgaagatgaaggtgaccctgtcccctgtcatcagccccgctcagcagaccaagactatgttcggccacactgctatcgacctggacggagcctggaccactaacacctggctgcaggacgacccctacgccgaatccagcactaggaaggtcatgacccagccattctctcactcaagatacttcactccaaagcctctgctggctggaaccacttccgcccaccctggacagtctctgttcttcttctcccgccccaccccatggctgaacacttacgaccctaccgtgcagtggggtgccctgctgtggtctatctacgtccccgagaagactggtatgaccgacttctacggcaccaaggaagtgtggatcaggtacaagtcagtcctgtga

SEQ ID NO: 7 - protein, 214 aa, Porcine circovirus 3
MRHRAIFRRRPRPRRRRRHRRRYARRRLFIRRPTAGTYYTKKYSTMNVISVGTPQNNKPWHANHFITRLNEWETAITFEYYKILKMKVTLSPVISPAQQTKTMFGHTAIDLDGAWTTNTWLQDDPYAESSTRKVMTQPFSHSRYFTPKPLLAGTTSAHPGQSLFFFSRPTPWLNTYDPTVQWGALLWSIYVPEKTGMTDFYGTKEVWIRYKSVL

SEQ ID NO: 8 - DNA, 735 bp, Porcine circovirus 3
  source 1..735: mol_type = unassigned DNA
atgcgccaccgtgctatcttccgccgtaggccaaggcctagacgccgtaggagacaccgccgtcgttacgctagacgccgtctgttcatcaggagacctaccgccggaacttactacaccaagaagtactctactatgaacgtgatctcagtcggtacccctcagaacaacaagccatggcacgctaaccacttcatcactcgcctgaacgagtgggaaactgccatcaccttcgagtactacaagatcctgaagatgaaggtgaccctgtctccagtcatctcacctgctcagcagaccaagactatgttcggtcacactgctatcgacctggacggcgcctggaccactaacacctggctgcaggacgacccctacgccgaatccagcactaggaaggtcatgacctccaagaagaagcactcaagatacttcactcccaagccactgctggctggcaccacttctgcccacccaggacagtccctgttcttcttctcccgccctaccccctggctgaacacttacgaccctactgtgcagtggggcgccctgctgtggtccatctacgtccctgagaagactggaatgaccgacttctacggtaccaaggaagtctggatcaggtacaagagcgtgctggtcaagatcaacatcaacctgactcctcccgtggctacttctcgtgtgccaagcagagctctgccactgaggttcggttgcggccaccgttga

SEQ ID NO: 9 - protein, 244 aa, Porcine circovirus 3
MRHRAIFRRRPRPRRRRRHRRRYARRRLFIRRPTAGTYYTKKYSTMNVISVGTPQNNKPWHANHFITRLNEWETAITFEYYKILKMKVTLSPVISPAQQTKTMFGHTAIDLDGAWTTNTWLQDDPYAESSTRKVMTSKKKHSRYFTPKPLLAGTTSAHPGQSLFFFSRPTPWLNTYDPTVQWGALLWSIYVPEKTGMTDFYGTKEVWIRYKSVLVKININLTPPVATSRVPSRALPLRFGCGHR

SEQ ID NO: 10 - protein, 244 aa, Porcine circovirus 3
MRHRAIFRRRPRPRRRRRHRRRYARRRLFIRRPTAGTYYTKKYSTMNVISVGTPQNNKPWHANHFITRLNEWETAITFEYYKILKMKVTLSPVISPAQQTKTMFGHTAIDLDGAWTTNTWLQDDPYAESSTRKVMTQPFSHSRYFTPKPLLAGTTSAHPGQSLFFFSRPTPWLNTYDPTVQWGALLWSIYVPEKTGMTDFYGTKEVWIRYKSVLVKININLTPPVATSRVPSRALPLRFGCGHR

SEQ ID NO: 11 - protein, 4 aa, Porcine circovirus 3
SKKK

SEQ ID NO: 12 - protein, 4 aa, Porcine circovirus 3
QPFS

SEQ ID NO: 13 - protein, 12 aa, Porcine circovirus 2
STIDYFQPNNKR

SEQ ID NO: 14 - protein, 5 aa, Porcine circovirus 3
SKKKH

SEQ ID NO: 15 - protein, 4 aa, Porcine circovirus 3
KKKH

SEQ ID NO: 16 - protein, 30 aa, Porcine circovirus 3
VKININLTPPVATSRVPSRALPLRFGCGHR

SEQ ID NO: 17 - protein, 6 aa, Porcine circovirus 3
QPFSYH

SEQ ID NO: 18 - protein, 5 aa, Porcine circovirus 3
LSRGF

SEQ ID NO: 19 - protein, 5 aa, Porcine circovirus 3
MASGF

SEQ ID NO: 20 - protein, 10 aa, Porcine circovirus 3
EFNLKDPPLN

SEQ ID NO: 21 - protein, 19 aa, Porcine circovirus 3
QFAPNNPSTEFDYETGRQL

SEQ ID NO: 22 - protein, 227 aa, synthetic construct
  REGION 1..227: note = Description of Artificial Sequence: Synthetic polypeptide
  VARIANT 14: note = MOD_RES - R, I, Y, A, L, K, or W
  VARIANT 15: note = MOD_RES - P, A, T, R, or F
  VARIANT 26: note = MOD_RES - W, R, A, K or absent
  VARIANT 32: note = MOD_RES - F, Y, H, or A
  VARIANT 36: note = MOD_RES - P, R, S, N, or Q
  VARIANT 43: note = MOD_RES - T, L, V, F, or G
  VARIANT 46: note = MOD_RES - Y, S, K, T, R, V, or A
  VARIANT 54: note = MOD_RES - V, K, R, N, P, Q, F, or A
  VARIANT 58: note = MOD_RES - Q, V, T, G, S, N, or A
  VARIANT 59: note = MOD_RES - N, T, R, Q, S, P, K, G, or E
  VARIANT 69: note = MOD_RES - I, R, T, N, S, E, or A
  VARIANT 78: note = MOD_RES - P, N, A, M, W, Y or absent
  VARIANT 83: note = MOD_RES - I, L, S, H, W, Y, K or absent
  VARIANT 84: note = MOD_RES - A, S, T, L, R, H or absent
  VARIANT 85: note = MOD_RES - I, V, L, P, Y, or S
  VARIANT 99: note = MOD_RES - L, F, M, V, A, I
  VARIANT 102: note = MOD_RES - V, C, T, I, L, A, R, or K
  VARIANT 105: note = MOD_RES - A, I, Y, V, R, S, P, or G
  VARIANT 108: note = MOD_RES - T, G, A, Q, R, M, E, or P
  VARIANT 109: note = MOD_RES - K, D, T, S, V, E, W, or P
  VARIANT 114: note = MOD_RES - H, S, N, R, or T
  VARIANT 123: note = MOD_RES - N, E, T, V, Q or absent
  VARIANT 124: note = MOD_RES - A, F, K, R, P, D, or T
  VARIANT 127: note = MOD_RES - T, K, E, N, S, D, or P
  VARIANT 128: note = MOD_RES - N, A, R, T, K, or G
  VARIANT 129: note = MOD_RES - T, N, A, G, or Q
  VARIANT 139: note = MOD_RES - S, Y, F, R, W, M, L, or H
  VARIANT 144: note = MOD_RES - V, T, K, S, R, or M
  VARIANT 148: note = MOD_RES - K, P, S, R, A, H, or I
  VARIANT 160: note = MOD_RES - L, V, Q, M, T, S, or D
  VARIANT 163: note = MOD_RES - S, T, G, I, A, V or absent
  VARIANT 166: note = MOD_RES - P, I, Q, V, or T
  VARIANT 169: note = MOD_RES - S, F, A, N, T, or V
  VARIANT 172: note = MOD_RES - F, N, L, T, W, or A
  VARIANT 176: note = MOD_RES - P, N, R, K, G, or A
  VARIANT 177: note = MOD_RES - T, Q, H, S, or R
  VARIANT 181: note = MOD_RES - N, R, P, S, or D
  VARIANT 184: note = MOD_RES - D, T, G, S, Q, K, A, or N
  VARIANT 185: note = MOD_RES - S, T, G, N, K or absent
  VARIANT 189: note = MOD_RES - Q, D, K, R, P, V, or T
  VARIANT 198: note = MOD_RES - Y, E, A, P, or R
  VARIANT 202: note = MOD_RES - K, I, Q, D, P, T, or N
  VARIANT 203: note = MOD_RES - T, Y, M, N or absent
  VARIANT 204: note = MOD_RES - M, D, P, T, S, I, Q, or A
  VARIANT 210: note = MOD_RES - T, R, Q, K, D, or Y
  VARIANT 219: note = MOD_RES - S, E, or Q
  VARIANT 223: note = MOD_RES - K, N, D, E, T, L, I or absent
  VARIANT 227: note = MOD_RES - L, T, H, Q, A or absent
MWLTRRRFRRRRRXXRRRRRHRRRYXRRRRRXRRRXTNGIFNXRLXRTFGFTWXKTTXXTLSWNADHLXFNLDDFLPXGPGSXXXPFEYYRIRKVKVEXRPXNPXTQXXRGFGXTAVILDGDXXFTXXXPLTYDPLANXSSRKXWKLXRGFKRYFTPKPXLDXANXSAXLPXNSRXXLWLXLQXXPNVXHYGLGFSFXQPEXXXQDYQIXITLYVQFRXFNLXDPPX

SEQ ID NO: 23 - protein, 214 aa, Porcine circovirus 3
MRHRAIFRRRPRPRRRRRHRRRYARRRLFIRRPTAGTYYTKKYSTMNVISVGTPQNNKPWHANHFITRLNEWETAITFEYYKILKMKVTLSPVISPAQQTKTMFGHTAIDLDGAWTTNTWLQDDPYAESSTRKVMTSKKKHSRYFTPKPLLAGTTSAHPGQSLFFFSRPTPWLNTYDPTVQWGALLWSIYVPEKTGMTDFYGTKEVWIRYKSVL

SEQ ID NO: 24 - protein, 233 aa, Porcine circovirus 2
MTYPRRRYRRRRHRPRSHLGQILRRRPWLVHPRHRYRWRRKNGIFNTRLSRTFGYTVKATTVTTPSWAVDMMRFNIDDFVPPGGGTNKISIPFEYYRIRKVKVEFWPCSPITQGDRGVGSTAVILDDNFVTKATALTYDPYVNYSSRHTIPQPFSYHSRYFTPKPVLDSTIDYFQPNNKRNQLWLRLQTSRNVDHVGLGTAFENSKYDQDYNIRVTMYVQFREFNLKDPPLEP

SEQ ID NO: 25 - protein, 234 aa, Porcine circovirus 2
MTYPRRRFRRRRHRPRSHLGQILRRRPWLVHPRHRYRWRRKNGIFNTRLSRTIGYTVKKTTVRTPSWNVDMMRFNINDFLPPGGGSNPLTVPFEYYRIRKVKVEFWPCSPITQGDRGVGSTAVILDDNFVTKANALTYDPYVNYSSRHTITQPFSYHSRYFTPKPVLDRTIDYFQPNNKRNQLWLRLQTTGNVDHVGLGTAFENSIYDQDYNIRITMYVQFREFNLKDPPLNPK

SEQ ID NO: 26 - protein, 247 aa, Beak and feather disease virus
MWGTSNCPCAIFQIRRIARPRYRRRHIRRYRPRRTYFRRRRFSTNRIYTLRLKRQFKFEIRKQTTQPGNLIWNADYMTFTLENFLTNTPNPSALNFEDYRIKLAKMEMKPTWGHYSIATEGFGHTAVIQDSRIEKFKTRADQSQDPLAPFDGAKKWYLSRGFKRLLRPKPQIMMNDLSTANQSAALWLNSVRTGWIPLQGGPNAAGSKVKHYGLAFSFAQPEIPIPYVCQLTIYVQFRQFAPNNPST

SEQ ID NO: 27 - protein, 247 aa, Beak and feather disease virus
MWGTSNCACATFQIRRRYARPYRRRHIRRYRRRRRHFRRRRFSTNRIYTLRLTRQFQFKINKQTTSVGNLIFNADYITFALDDFLQAVPNPHTLNFEDYRIKLAKMEMRPTGGHYTVQSDGFGHTAVIQDSRITRFKTTADQTQDPLAPFDGAKKWFVSRGFKRLLRPKPQITIEDLTTANQSAALWLNSARTGWIPLQGGPNSAGTKVRHYGIAFSFPQPEQTITYVTKLTLYVQFRQFAPNNPST

SEQ ID NO: 28 - protein, 281 aa, unidentified
  REGION 1..281: note = Description of Unknown: Bat circovirus sequence
MTAHAQGGGARHASAMFLFLEMARWHTRRWRRATLHAVARSHRRRRHAMGGRRRRHRRRSTYKFFHVRLTRYYTVLWPKATTPSDDTETTYGWNLDHVNFKLSDFLPMDSSGRPSLPAFKDYNITKAVVRVKPINVPVSMRVEQYGNHATDFDGTDVGIGTVHTSGDPKPSPNNETGPKTSDPLRNRTSRKSWNVRTGFTRILKPTVVAQTANCCGIGPGSNFITRGLKHAWLRLDSNGVKTPWNGLSISLREGDQSLLTQYTITLYVKFREFDLDFNPHA

SEQ ID NO: 29 - protein, 270 aa, Canine circovirus
MRVRRHARASRRSYRTRPLNRYRRRRQNRFKLFHLRLRRTLTADWPTAPVKPTNDPQTETPLLWNFDHLSFKLTDFLQASHGTGDFQHLPPFRFYKFKKVYIRARWINWPRTLMENVLGRTALDLDGEDQGRGNATRSHLDPGTVPGRLEPPKDPNKAPFIYDPLQDRSSSRSFNMASGFKRGLTPKPMFTQEIASPSATAPWLTRGTPWVSVIQGANMVWNGLSISLRQMKDMRPTTPDTSTSQIPQVQYDISAYIAFKEFDYETGRQL

SEQ ID NO: 30 - protein, 250 aa, Canary circovirus
  VARIANT 28: note = MOD_RES - Any amino acid
MWLTFNQVARRRRPLAPRRRRWRRRYWXRRRRIPANRRGHRTNRVYRFRFVREFGQVLQKGTGGSQLSFGTDGINIILDDFLDWGTINWRLPFEDYRIRLAKVEMRPLNESWEEWKGFGHNVPIQDNHLEDFFKKTRLDADPLANWDGARKWDLRKGFKRLFKPRPQLSVTDTDAANVTAALWLNNPKSLWIPIMKKSDQNLPSSGTRVKHYGLAFSWPEPTPNQMDYQVKVTIYCEFRQMNLTHLATPK

SEQ ID NO: 31 - protein, 250 aa, Goose circovirus
MPLYRARPRSLYSRRRRATNRRRRYRRRRLHIGRIRSKYTIFNVKQTQNISFTFFGTGSPDKNKWQAMSLEAVQSSGTSPKPGINLRFAVFGDRLPGTGNQYHYPFDYYMIRMVKVELRPAFNPFQRVRTQGSTYIDKEGNITTTTSGGEWNVDPYAAMSSRKTWSPHRYHKRVFVPKPTIQQGGTGTNIWSTWYTPGGRQLWLNSIQDNVVFYGMGMSLRQAEDTAAPLTVEATITYYIRFGQWTGLSP

SEQ ID NO: 32 - protein, 231 aa, unidentified
  REGION 1..231: note = Description of Unknown: Bat circovirus sequence
MRRKFRRFRRKFKKFSRRFKRHFGGKRRKTTRQVQFKFKVQTVPYLNGSIAPSSSINWNNTSNTASHYTFAFTLGDIPHYSDLSSVFDAAKLAAVKLKFVPRYTMGQLPTSASTTYANTSTPCVVVKDYDDANPLTSYANALLYQNARVVSILKPFSVYLKPKLSGGVENTSLVIVAQSQARPWLDSGATAVPYYGVKLEVPGINTTQMLGQAIWDIYGTYYVKLKQIRLL

SEQ ID NO: 33 - protein, 238 aa, Bat associated circovirus 3
MPIRRRSRYSRRRRWRRNTRRRRVARGAYRWRRKNGIINVRLSATKDWTMASTTAEGYNVARLEVNLRQFMPAGPGSAINTKSIPWAYYRIRKMKFEILPKMIPAQTPYRYGSTAIYLGMQAPAPTQGKTYDPHLKHVKQNMSGLITDQLKRYFTPKPDLDSITSTAWFQPNNKANQVWINMTNDNITHGQVGWSMERISNMAQNFKIRVTLYVQFREFNLIDYPAQAPLLVDEEPSE

SEQ ID NO: 34 - protein, 12 aa, Porcine circovirus 3
EFNLKDPPLNPK

SEQ ID NO: 35 - protein, 9 aa, Porcine circovirus 3
QFAPNNPST

SEQ ID NO: 36 - protein, 10 aa, Porcine circovirus 3
EFDYETGRQL

SEQ ID NO: 37 - protein, 33 aa, Porcine circovirus 3
SVLVKININLTPPVATSRVPSRALPLRFGCGHR